(19) United States
(12) Patent Application Publication
(10) Pub. No.: US 2023/0212170 A1
(43) Pub. Date: Jul. 6, 2023

(54) INHIBITORS OF KRAS G12C PROTEIN AND USES THEREOF

(71) Applicants: SHANGHAI ANTENGENE CORPORATION LIMITED, Changning District (CN); ANTENGENE DISCOVERY LIMITED, Shanghai (CN)

(72) Inventors: Bo SHAN, Shanghai (CN); Jian WANG, Shanghai (CN); Bing HOU, Shanghai (CN); Zhongyang SHI, Shanghai (CN); Peng CHEN, Shanghai (CN); Hui YUWEN, Shanghai (CN); Xingquan MA, Shanghai (CN); Zhu DU, Shanghai (CN); Jay MEI, Shanghai (CN)

(21) Appl. No.: 18/000,513

(22) PCT Filed: Jun. 3, 2021
(86) PCT No.: PCT/CN2021/098083

§ 371 Date: Dec. 2, 2022

(30) Foreign Application Priority Data
Jun. 4, 2020 (WO) PCT/CN2020/094307
Feb. 5, 2021 (WO) PCT/CN2021/075503
Apr. 27, 2021 (WO) PCT/CN2021/090216
May 26, 2021 (CN) CT/CN2021/096134

(51) Int. Cl. C07D 487/04; C07D 513/04; C07D 403/14; C07D 471/04; C07D 471/18; C07F 9/6584

(52) U.S. Cl. CPC C07D 487/04; C07D 513/04; C07D 403/14; C07D 471/04; C07D 471/18; C07F 9/65846

(57) ABSTRACT

Provided are novel compounds useful as inhibitors of the KRAS protein, as well as pharmaceutical compositions comprising these compounds and methods of treatment by administration of these compounds or the pharmaceutical compositions.

### FIELD OF THE DISCLOSURE

The present disclosure generally relates to novel compounds useful as inhibitors of the KRAS protein, as well as pharmaceutical compositions comprising these compounds and methods of treatment by administration of these compounds or the pharmaceutical compositions.

### BACKGROUND OF THE DISCLOSURE

The KRAS oncoprotein is a GTPase and an essential mediator of intracellular signaling pathways that are involved in tumor cell growth and survival. In normal cells, KRAS functions as a molecular switch, alternating between inactive GDP-bound and active GTP-bound states. Transition between these states is facilitated by guanine nucleotide-exchange factors which load GTP and activate KRAS and GTP hydrolysis, which is catalyzed by GTPase-activating proteins to inactivate KRAS. GTP binding to KRAS promotes binding of effectors to trigger signal transduction pathways including the RAF-MEK-ERK (MAPK) pathway.

Activating mutations in KRAS are a hallmark of cancer and prevent the association of GTPase-activating proteins, thus stabilizing effector binding and enhancing KRAS signaling. KRAS G12C is present in approximately 13% of lung adenocarcinoma, 3% of colorectal cancer and 2% of other solid tumors. Thus, KRAS, in particular KRAS G12C, is widely considered an oncology target of exceptional importance.

While progress has been made for targeting KRAS G12C, targeting this gene with small molecules is still a challenge. Accordingly, there is a need in the art to develop improved small molecule compounds that inhibit KRAS, in particular KRAS G12C.

### SUMMARY OF THE DISCLOSURE

The present disclosure provides compounds, including stereoisomers, pharmaceutically acceptable salts, tautomers and prodrugs thereof, which are capable of modulating KRAS G12C proteins. Methods for use of such compounds for treatment of various diseases or conditions, such as cancer, are also provided.

In one aspect, the present disclosure provides a compound having Formula (I):

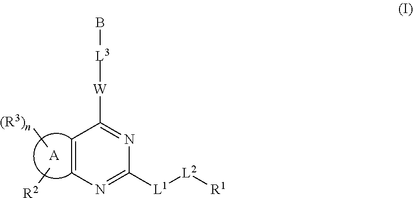

or a pharmaceutically acceptable salt thereof,

wherein

Ring A is selected from the group consisting of saturated or partially unsaturated cycloalkyl, saturated or partially unsaturated hetercyclyl, and heteroaryl;

L¹ is a bond, O, S or N(R^{a});

L² is selected from the group consisting of a bond, alkyl, alkenyl, alkynyl, heteroalkyl, heteroalkenyl, and heteroalkynyl;

R¹ is selected from the group consisting of alkyl, alkenyl, alkynyl, heteroalkyl, heteroalkenyl, heteroalkynyl, saturated or partially unsaturated cycloalkyl, saturated or partially unsaturated hetercyclyl, aryl, and heteroaryl, wherein each of alkyl, alkenyl, alkynyl, cycloalkyl, hetercyclyl, aryl, heteroaryl is optionally substituted with one or more R^{b};

R² is selected from the group consisting of H, alkyl, alkenyl, alkynyl, heteroalkyl, heteroalkenyl, heteroalkynyl, saturated or partially unsaturated cycloalkyl, saturated or partially unsaturated hetercyclyl, aryl and heteroaryl, wherein each of alkyl, alkenyl, alkynyl, heteroalkyl, heteroalkenyl, heteroalkynyl, cycloalkyl, hetercyclyl, aryl and heteroaryl is optionally substituted with one or more R^{c},

R³ is selected from the group consisting of hydrogen, oxo, halogen, cyano, hydroxyl, —NR^{d}R^{e}, —C(O)NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R; or

R⁴ and R⁵, R⁴ and R⁶, R⁴ and R⁷, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl;

W is saturated or partially unsaturated cycloalkyl, or saturated or partially unsaturated heterocyclyl, wherein each of cycloalkyl and heterocyclyl is optionally substituted with one or more R^{g},

L³ is a bond, alkyl or —NR^{d}—;

B is an electrophilic moiety capable of forming a covalent bond with a cysteine residue at position 12 of a K-Ras G12C mutant protein;

R^{a} is independently hydrogen or alkyl;

each R^{b} is independently selected from the group consisting of oxo, cyano, halogen, hydroxy, acyl, —NR^{d}R^{e}, carbamoyl, carboxyl, alkyl, alkenyl, alkynyl, alkoxyl, alkoxylalkyl, cycloalkylalkyl, cycloalkyl, heterocyclyl, aryl, heteroaryl;

each R^{c} is independently selected from the group consisting of oxo, halogen, cyano, hydroxy, —NR^{d}R^{e}, —C(O)OR^{a}, —C(O)N(R^{d})(R^{e}), alkyl, alkenyl, alkynyl, heteroalkyl, heteroalkenyl, heteroalkynyl, alkoxyl, saturated or partially unsaturated cycloalkyl, saturated or partially unsaturated heterocyclyl, aryl, and heteroaryl;

each of R^{a} and R^{e} is independently selected from the group consisting of hydrogen, alkyl, alkenyl, alkynyl, heteroalkyl, heteroalkenyl, heteroalkynyl, aryl, heteroaryl, wherein each of alkyl, alkenyl, alkynyl, heteroalkyl, heteroalkenyl, heteroalkynyl, aryl and heteroaryl is optionally substituted with cyano, halogen, hydroxy, or amino;

each R^{f} is independently selected from the group consisting of oxo, halogen, cyano, hydroxy, —NR^{c}R^{d}, alkyl, alkenyl, alkynyl, heteroalkyl, heteroalkenyl, heteroalkynyl, cycloalkyl, heterocyclyl, aryl, and heteroaryl;

each R^{g} is independently selected from the group consisting of oxo, cyano, halogen, hydroxy, —NR^{d}R^{e}, carbamoyl, carboxy, alkyl, alkenyl, alkynyl, heteroalkyl, heteroalkenyl, heteroalkynyl, saturated or partially unsaturated cycloalkyl, and saturated or partially unsaturated heterocyclyl, wherein each of alkyl, alkenyl, alkynyl, heteroalkyl, heteroalkenyl, heteroalkynyl, cycloalkyl, and heterocyclyl is optionally substituted with cyano, halogen, hydroxy, —NR^{d}R^{e}, carboxy, carbamoyl, haloalkyl, aryl or heteroaryl;

n is 0, 1, 2, 3 or 4.

In some embodiments, the present disclosure provides compound having a formula selected from the group consisting of:

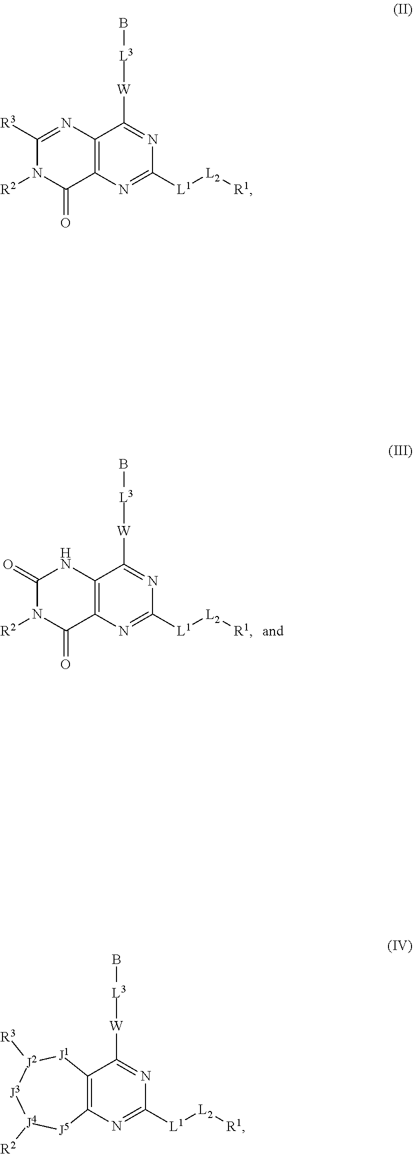

or pharmaceutically acceptable salts thereof,

wherein

J¹ is absent, CH(R⁴), NR⁴, SO₂ or P(O)CH₃;

J² is absent, CR⁵, N, SO₂ or P(O)CH₃;

J³ is absent, CH(R⁶), NR⁶, SO₂ or P(O)CH₃;

J⁴ is absent, CR⁷, N, SO₂ or P(O)CH₃;

J⁵ is absent, CH(R⁸), NR⁸, SO₂ or P(O)CH₃;

R⁴, R⁵, R⁶, R⁷ and R⁸ are each independently selected from the group consisting of hydrogen, oxo, halogen, cyano, hydroxyl, —NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R^{f}; or

R² and any one of R⁴, R⁵, R⁶, R⁷ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R³ and any one of R⁴, R⁵, R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R⁴ and any one of R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl.

In another aspect, the present disclosure provides a pharmaceutical composition comprising the compound of the present disclosure or a pharmaceutically acceptable salt thereof, and a pharmaceutically acceptable excipient.

In a further aspect, the present disclosure provides a method for treating cancer, comprising administering an effective amount of a compound of the present disclosure or a pharmaceutically acceptable salt thereof or the pharmaceutical composition of the present disclosure to a subject in need thereof.

In a further aspect, the present disclosure provides a method for treating cancer in a subject in need thereof, the method comprising:
(a) determining that the cancer is associated with KRAS G12C mutation; and
(b) administering to the subject an effective amount of a compound of the present disclosure or a pharmaceutically acceptable salt thereof or the pharmaceutical composition of the present disclosure.

In a further aspect, the present disclosure provides a method for inhibiting tumor metastasis, comprising administering an effective amount of a compound of the present disclosure or a pharmaceutically acceptable salt thereof or the pharmaceutical composition of the present disclosure to a subject in need thereof.

In a further aspect, the present disclosure provides a method for regulating activity of a KRAS G12C mutant protein, comprising reacting the KRAS G12C mutant protein with a compound of the present disclosure or a pharmaceutically acceptable salt thereof or the pharmaceutical composition of the present disclosure.

In a further aspect, the present disclosure provides a method for preparing a labeled KRAS G12C mutant protein, comprising reacting the KRAS G12C mutant protein with a compound of the present disclosure or a pharmaceutically acceptable salt thereof, to result in the labeled KRAS G12C mutant protein.

In a further aspect, the present disclosure provides use of a compound of the present disclosure or a pharmaceutically acceptable salt thereof or the pharmaceutical composition of the present disclosure, in the manufacture of a medicament for treating cancer.

In a further aspect, the present disclosure provides use of a compound of the present disclosure or a pharmaceutically acceptable salt thereof or the pharmaceutical composition of the present disclosure, in the manufacture of a medicament for inhibiting tumor metastasis.

In a further aspect, the present disclosure provides a compound of the present disclosure or a pharmaceutically acceptable salt thereof or the pharmaceutical composition of the present disclosure, for treating cancer.

In a further aspect, the present disclosure provides a compound of the present disclosure or a pharmaceutically acceptable salt thereof or the pharmaceutical composition of the present disclosure, for inhibiting tumor metastasis.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. It is specifically intended that each linking substituent includes both the forward and backward forms of the linking substituent. For example, —NR(CR′R″)— includes both —NR(CR′R″)— and —(CR′R″)NR—. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R^{I}) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein the term “acyl” refers to —C(═O)—R, wherein R is a substituent such as hydrogen, alkyl, cycloalkyl, aryl or heterocyclyl, wherein the alkyl, cycloalkyl, aryl and heterocyclyl are as defined herein.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “alkoxylalkyl” refers to a radical of the formula —R″OR′, wherein R′ and R″ are independently an alkyl as defined above.

As used herein, the term “amino” refers to —NH₂ group. Amino groups may also be substituted with one or more groups such as alkyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “carbamoyl” refers to —C(O)NH₂.

As used herein, the term “carboxy” refers to —COOH.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “cycloalkylalkyl” refers to a radical of formula —R′R″, wherein R′ is an alkyl as defined above, and R″ is a cycloalkyl as defined above.

As used herein, the term “cyano” refers to —CN.

As used herein, the term “halogen” refers to an atom selected from fluorine (or fluoro), chlorine (or chloro), bromine (or bromo) and iodine (or iodo).

As used herein, the term “haloalkyl” refers to an alkyl, as defined above, that is substituted by one or more halogens, as defined above. Examples of haloalkyl include, but are not limited to, trifluoromethyl, difluoromethyl, trichloromethyl, 2,2,2-trifluoroethyl, 1,2-difluoroethyl, 3-bromo-2-fluoropropyl, 1,2-dibromoethyl, and the like.

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” refers to —OH.

As used herein, the term “oxo” refers to ═O substituent.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

The present disclosure provides novel compounds of Formula (I) and pharmaceutically acceptable salts thereof, synthetic methods for making the compounds, pharmaceutical compositions containing them and various uses of the disclosed compounds.

In one aspect, the present disclosure provides a compound having Formula (I):

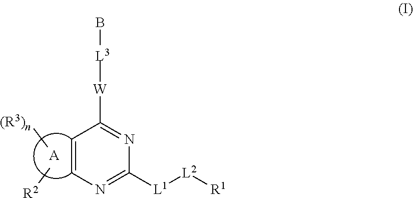

or a pharmaceutically acceptable salt thereof,

wherein

Ring A is selected from the group consisting of saturated or partially unsaturated cycloalkyl, saturated or partially unsaturated hetercyclyl, and heteroaryl;

L¹ is a bond, O, S or N(R^{a});

L² is selected from the group consisting of a bond, alkyl, alkenyl, alkynyl, heteroalkyl, heteroalkenyl, and heteroalkynyl;

R¹ is selected from the group consisting of alkyl, alkenyl, alkynyl, heteroalkyl, heteroalkenyl, heteroalkynyl, saturated or partially unsaturated cycloalkyl, saturated or partially unsaturated hetercyclyl, aryl, and heteroaryl, wherein each of alkyl, alkenyl, alkynyl, cycloalkyl, hetercyclyl, aryl, heteroaryl is optionally substituted with one or more R^{b};

R² is selected from the group consisting of H, alkyl, alkenyl, alkynyl, heteroalkyl, heteroalkenyl, heteroalkynyl, saturated or partially unsaturated cycloalkyl, saturated or partially unsaturated hetercyclyl, aryl and heteroaryl, wherein each of alkyl, alkenyl, alkynyl, heteroalkyl, heteroalkenyl, heteroalkynyl, cycloalkyl, hetercyclyl, aryl and heteroaryl is optionally substituted with one or more R^{c},

R³ is selected from the group consisting of hydrogen, oxo, halogen, cyano, hydroxyl, —NR^{d}R^{e}, —C(O)NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R; or

R⁴ and R⁵, R⁴ and R⁶, R⁴ and R⁷, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl;

W is saturated or partially unsaturated cycloalkyl, or saturated or partially unsaturated heterocyclyl, wherein each of cycloalkyl and heterocyclyl is optionally substituted with one or more R^{g},

L³ is a bond, alkyl or —NR^{d}—;

B is an electrophilic moiety capable of forming a covalent bond with a cysteine residue at position 12 of a K-Ras G12C mutant protein;

R^{a} is independently hydrogen or alkyl;

each R^{b} is independently selected from the group consisting of oxo, cyano, halogen, hydroxy, acyl, —NR^{d}R^{e}, carbamoyl, carboxyl, alkyl, alkenyl, alkynyl, alkoxyl, alkoxylalkyl, cycloalkylalkyl, cycloalkyl, heterocyclyl, aryl, heteroaryl;

each R^{c} is independently selected from the group consisting of oxo, halogen, cyano, hydroxy, —NR^{d}R^{e}, —C(O)OR^{a}, —C(O)N(R^{d})(R^{e}), alkyl, alkenyl, alkynyl, heteroalkyl, heteroalkenyl, heteroalkynyl, alkoxyl, saturated or partially unsaturated cycloalkyl, saturated or partially unsaturated heterocyclyl, aryl, and heteroaryl;

each of R^{d} and R^{e} is independently selected from the group consisting of hydrogen, alkyl, alkenyl, alkynyl, heteroalkyl, heteroalkenyl, heteroalkynyl, aryl, heteroaryl, wherein each of alkyl, alkenyl, alkynyl, heteroalkyl, heteroalkenyl, heteroalkynyl, aryl and heteroaryl is optionally substituted with cyano, halogen, hydroxy, or amino;

each R^{f} is independently selected from the group consisting of oxo, halogen, cyano, hydroxy, —NR^{c}R^{d}, alkyl, alkenyl, alkynyl, heteroalkyl, heteroalkenyl, heteroalkynyl, cycloalkyl, heterocyclyl, aryl, and heteroaryl;

each R^{g} is independently selected from the group consisting of oxo, cyano, halogen, hydroxy, —NR^{d}R^{e}, carbamoyl, carboxy, alkyl, alkenyl, alkynyl, heteroalkyl, heteroalkenyl, heteroalkynyl, saturated or partially unsaturated cycloalkyl, and saturated or partially unsaturated heterocyclyl, wherein each of alkyl, alkenyl, alkynyl, heteroalkyl, heteroalkenyl, heteroalkynyl, cycloalkyl, and heterocyclyl is optionally substituted with cyano, halogen, hydroxy, —NR^{d}R^{e}, carboxy, carbamoyl, haloalkyl, aryl or heteroaryl;

n is 0, 1, 2, 3 or 4.

In some embodiments, Ring A is saturated or partially unsaturated cycloalkyl.

In some embodiments, Ring A is saturated or partially unsaturated hetercyclyl.

In some embodiments, Ring A is heteroaryl.

In some embodiments, L¹ is O.

In some embodiments, L² is a bond.

In some embodiments, L² is alkyl.

In some embodiments, L² is methyl, ethyl or propyl.

In some embodiments, R¹ is saturated or partially unsaturated cycloalkyl, or saturated or partially unsaturated heterocyclyl, wherein each cycloalkyl and heterocyclyl is optionally substituted with one or more R^{b}. In certain embodiments, each R^{b} is selected from the group consisting of oxo, cyano, halogen, hydroxy, acyl, —NR^{d}R^{e}, alkyl, alkoxyl, alkoxylalkyl and cycloalkylalkyl.

In some embodiments, R¹ is saturated or partially unsaturated heterocyclyl selected from the group consisting of:

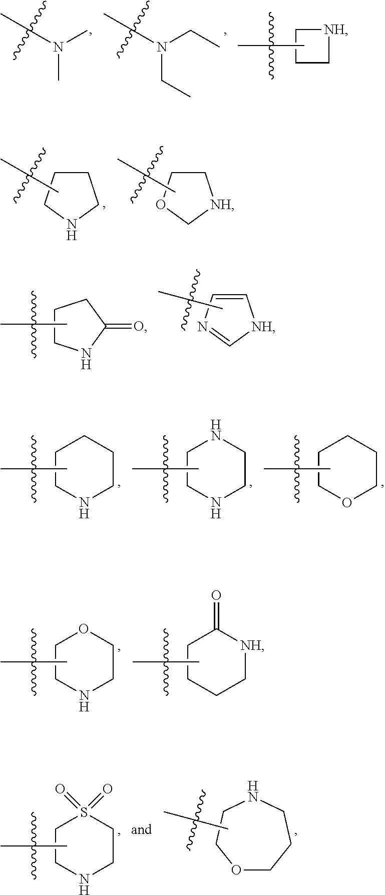

each of which is optionally substituted with one or more R^{b}.

In certain embodiments, each R^{b} is selected from the group consisting of oxo, halogen, acyl, —NR^{d}R^{e}, alkyl, alkoxyl, alkoxylalkyl, and cycloalkylalkyl. In certain embodiments, each R^{b} is halogen or alkyl. In certain embodiments, each R^{b} is fluoro, chloro or methyl.

In some embodiments, R¹ is

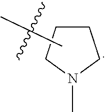

In some embodiments, -L¹-L²-R¹ is

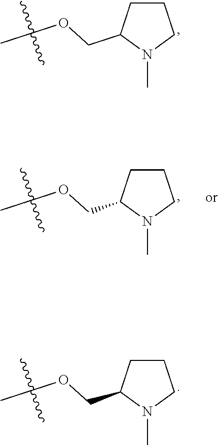

In some embodiments, R¹ is

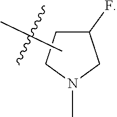

In some embodiments, -L¹-L²-R¹ is

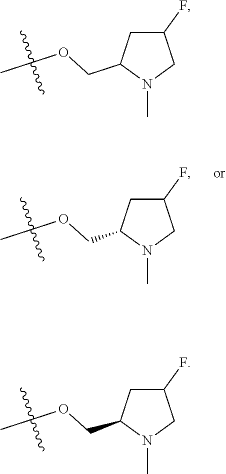

In some embodiments, R² is aryl optionally substituted with one or more R^{c}. In certain embodiments, each R^{c} is selected from the group consisting of halogen, cyano, hydroxyl, alkyl, alkenyl, alkoxyl, and saturated or partially unsaturated cycloalkyl.

In some embodiments, R² is aryl selected from the group consisting of:

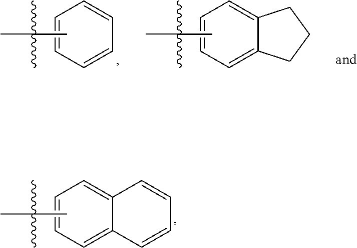

each of which is optionally substituted with one or more R^{c}.

In certain embodiments, each R^{c} is selected from the group consisting of halogen, hydroxyl, alkyl, alkenyl, alkoxyl, and saturated or partially unsaturated cycloalkyl. In certain embodiments, each R^{c} is selected from the group consisting of halogen, hydroxyl, alkyl, alkenyl, alkoxyl, and saturated cycloalkyl. In certain embodiments, each R^{c} is selected from the group consisting of fluoro, chloro, hydroxyl, methyl, ethyl, 2-methylpropenyl, methoxyl, and cyclopropyl.

In some embodiments, R² is selected from the group consisting of:

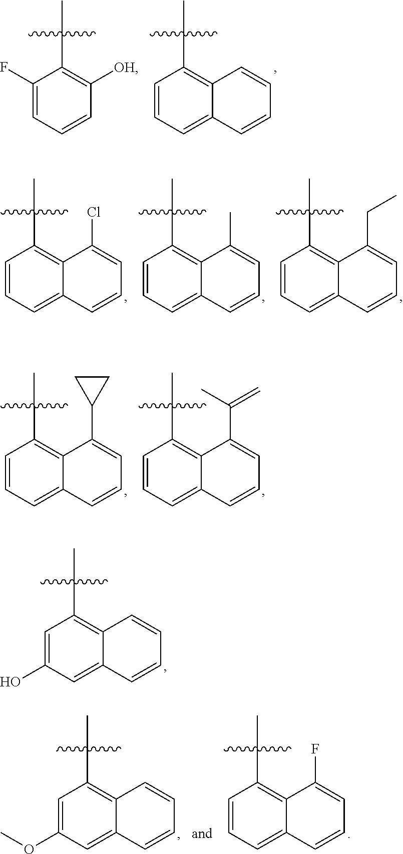

In some embodiments, R^{c} is heteroaryl optionally substituted with one or more R^{c}. In certain embodiments, each R^{c} is selected from the group consisting of halogen, cyano, hydroxyl, —NR^{d}R^{e}, alkyl, alkenyl, alkoxyl, and saturated or partially unsaturated cycloalkyl.

In some embodiments, R² is heteroaryl selected from the group consisting of:

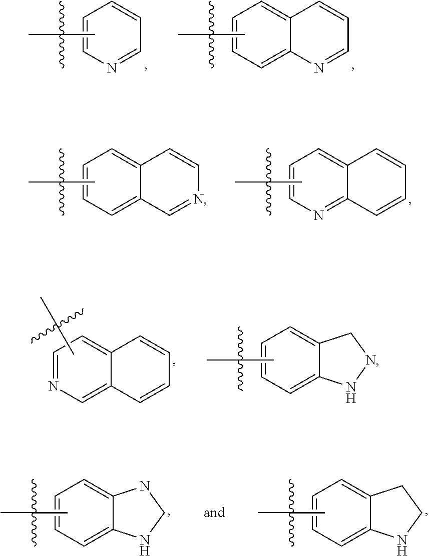

each of which is optionally substituted with one or more R^{c}.

In certain embodiments, each R^{c} is selected from the group consisting of halogen, cyano, hydroxyl, —NR^{d}R^{e}, alkyl, alkenyl, alkoxyl, and saturated or partially unsaturated cycloalkyl. In certain embodiments, each R^{c} is halogen or alkyl. In certain embodiments, each R^{c} is selected from the group consisting of fluoro, chloro, methyl, and ethyl.

In some embodiments, R² is selected from the group consisting of:

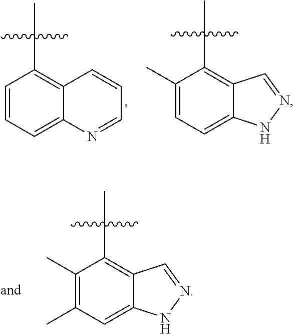

In some embodiments, R³ is selected from the group consisting of oxo, alkyl and aryl, wherein alkyl and aryl is optionally substituted with one or more R^{c}. In certain embodiments, R^{c} is selected from the group consisting of halogen, cyano, hydroxy, —NR^{c}R^{d}, alkyl.

In some embodiments, R³ is selected from the group consisting of oxo, methyl, ethyl, trifluoromethyl and phenyl.

In some embodiments, two R³, together with the atoms to which they are each attached, form saturated or partially unsaturated cycloalkyl optionally substituted with one or more substituents selected from the group consisting of cyano, halogen, hydroxy, and —NR^{c}R^{d}.

In some embodiments, W is saturated or partially unsaturated heterocyclyl optionally substituted with one or more R^{g}. In certain embodiments, R^{g} is alkyl optionally substituted with one or more substituents selected from the group consisting of cyano, halogen, and hydroxyl.

In some embodiments, W is heterocyclyl selected from the group consisting of:

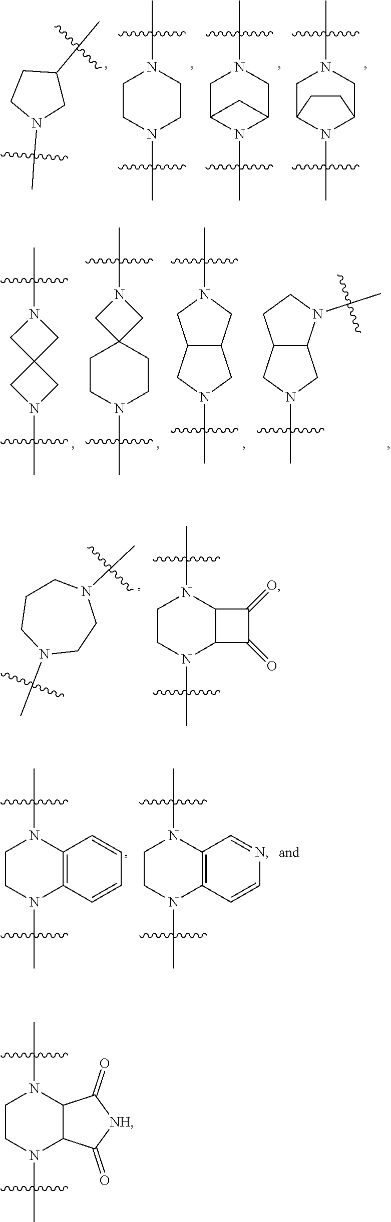

each of which is optionally substituted with one or more R^{g}.

In certain embodiments, each R^{g} is alkyl optionally substituted with cyano. In certain embodiments, each R^{g} is methyl optionally substituted with cyano.

In some embodiments, W is selected from the group consisting of:

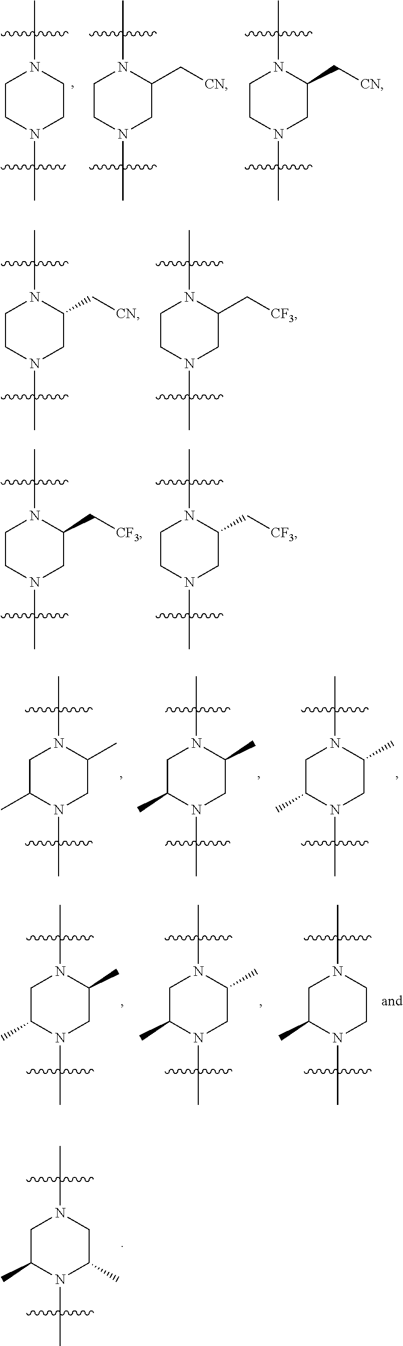

In some embodiments, L³ is a bond or —NR^{d}—.

In some embodiments, B is selected from the group consisting of:

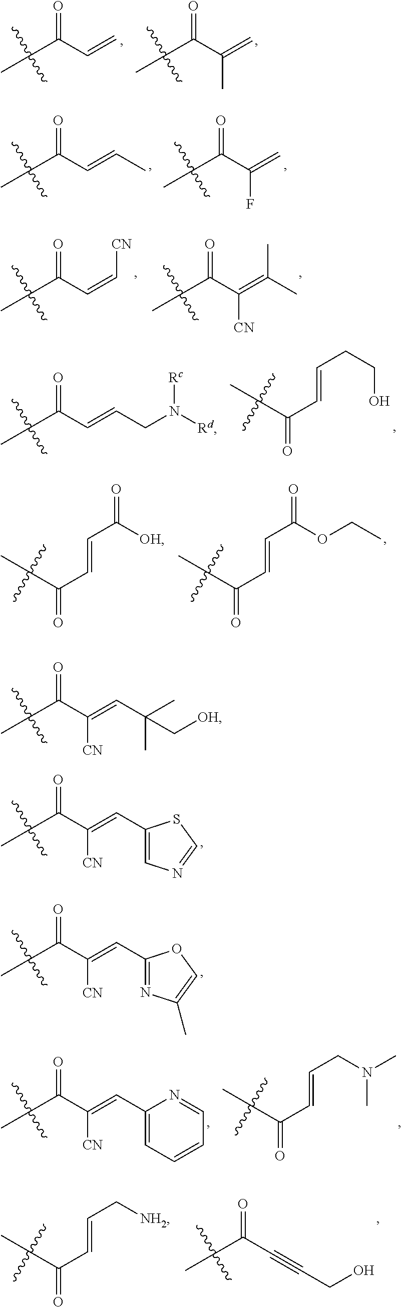

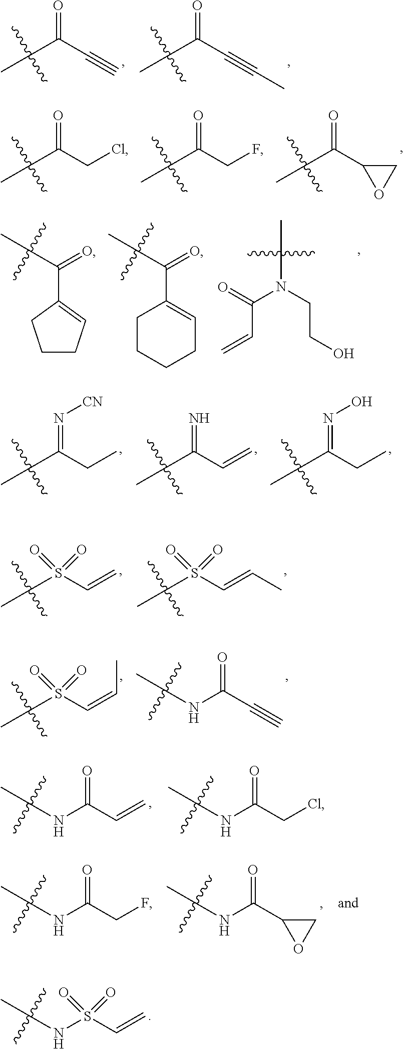

In some embodiments, the present disclosure provides a compound having a formula selected from the group consisting of:

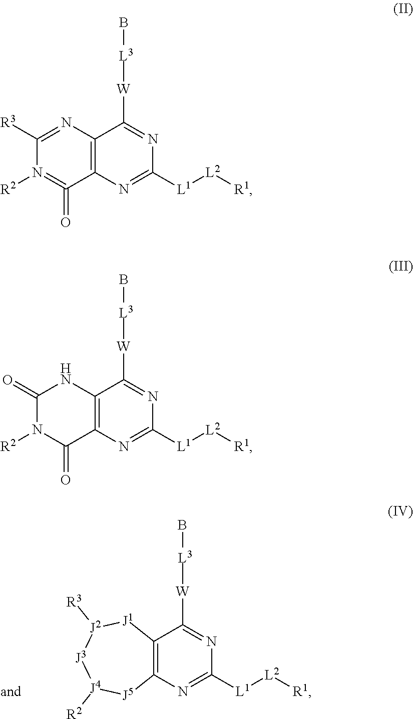

or a pharmaceutically acceptable salt thereof,

wherein

J¹ is absent, CH(R⁴), NR⁴, SO₂ or P(O)CH₃;

J² is absent, CR⁵, N, SO₂ or P(O)CH₃;

J³ is absent, CH(R⁶), NR⁶, SO₂ or P(O)CH₃;

J⁴ is absent, CR⁷, N, SO₂ or P(O)CH₃;

J⁵ is absent, CH(R⁸), NR⁸, SO₂ or P(O)CH₃;

R⁴, R⁵, R⁶, R⁷ and R⁸ are each independently selected from the group consisting of hydrogen, oxo, halogen, cyano, hydroxyl, —NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R^{f}; or

R² and any one of R⁴, R⁵, R⁶, R⁷ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R³ and any one of R⁴, R⁵, R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R⁴ and any one of R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or

R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl.

In some embodiments, the present disclosure provides a compound having a formula selected from the group consisting of:

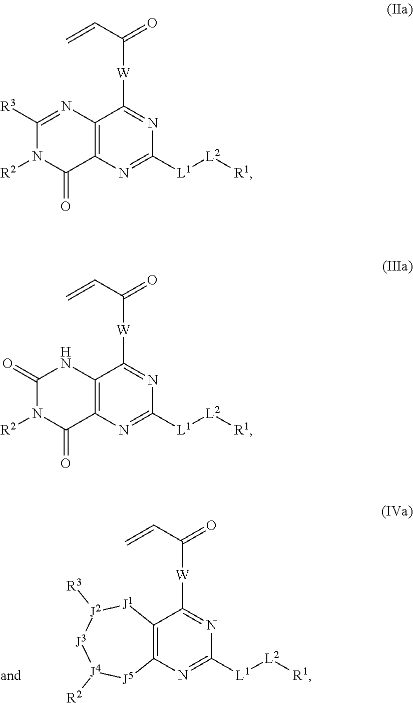

or a pharmaceutically acceptable salt thereof.

In some embodiments, the present disclosure provides a compound having a formula selected from the group consisting of:

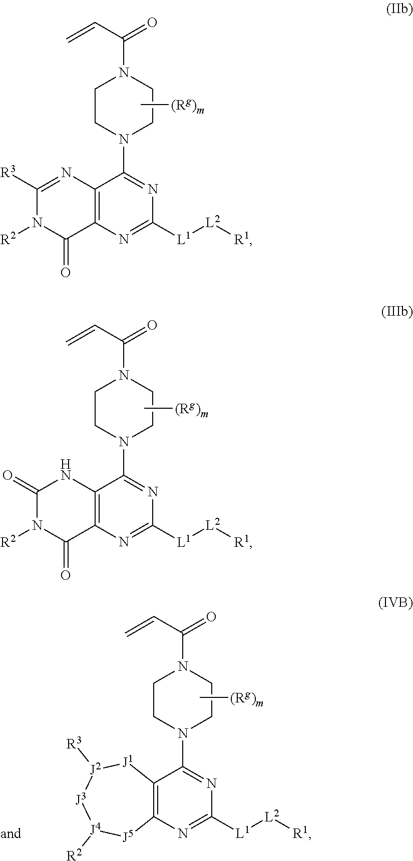

or a pharmaceutically acceptable salt thereof, wherein m is 0, 1, 2, 3 or 4.

In some embodiments, the present disclosure provides a compound having a formula selected from the group consisting of:

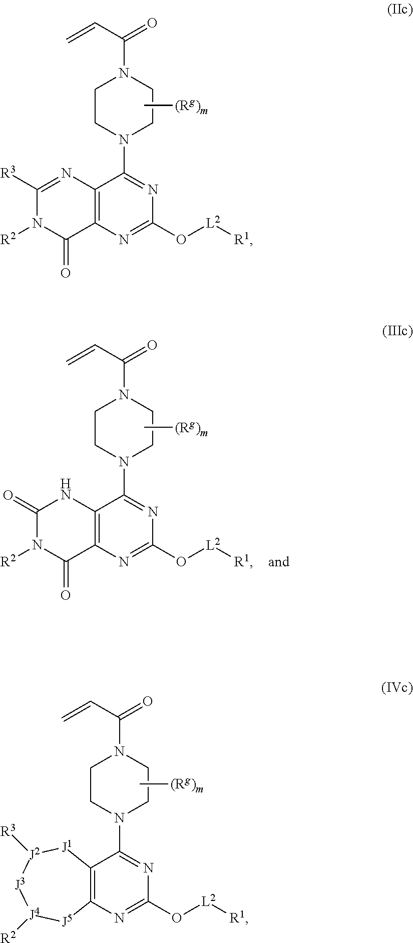

or a pharmaceutically acceptable salt thereof, wherein m is 0, 1, 2, 3 or 4.

In some embodiments, the present disclosure provides a compound having a formula selected from the group consisting of:

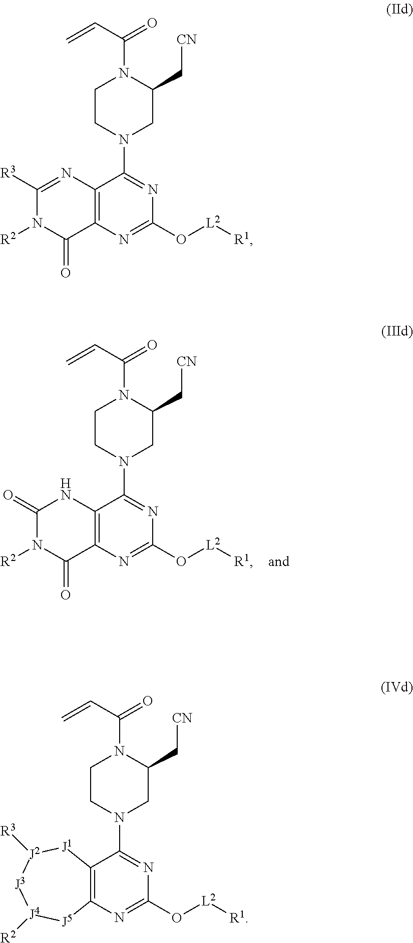

or a pharmaceutically acceptable salt thereof.

In some embodiments, the present disclosure provides a compound having a formula of:

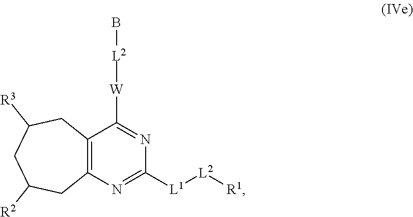

or a pharmaceutically acceptable salt thereof.

In some embodiments, the present disclosure provides a compound having a formula of:

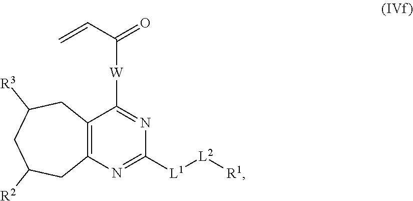

or a pharmaceutically acceptable salt thereof.

In some embodiments, the present disclosure provides a compound having a formula of:

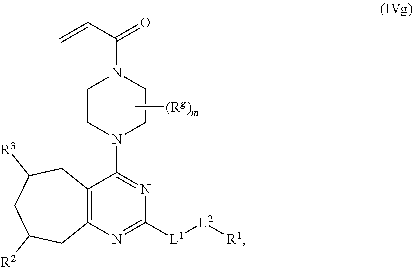

or a pharmaceutically acceptable salt thereof, wherein m is 0, 1, 2, 3 or 4.

In some embodiments, the present disclosure provides a compound having a formula of:

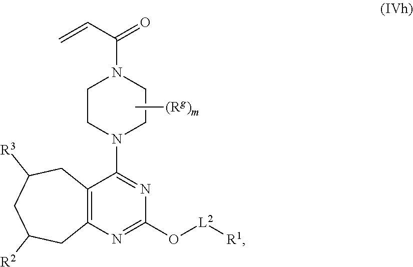

or a pharmaceutically acceptable salt thereof, wherein m is 0, 1, 2, 3 or 4.

In some embodiments, the present disclosure provides a compound having a formula of:

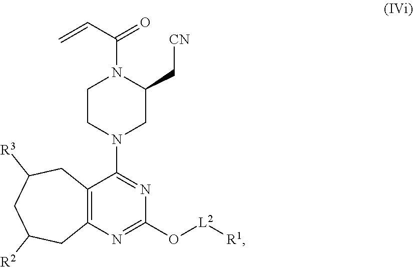

or a pharmaceutically acceptable salt thereof.

In some embodiments, the present disclosure provides a compound having a formula selected from the group consisting of:

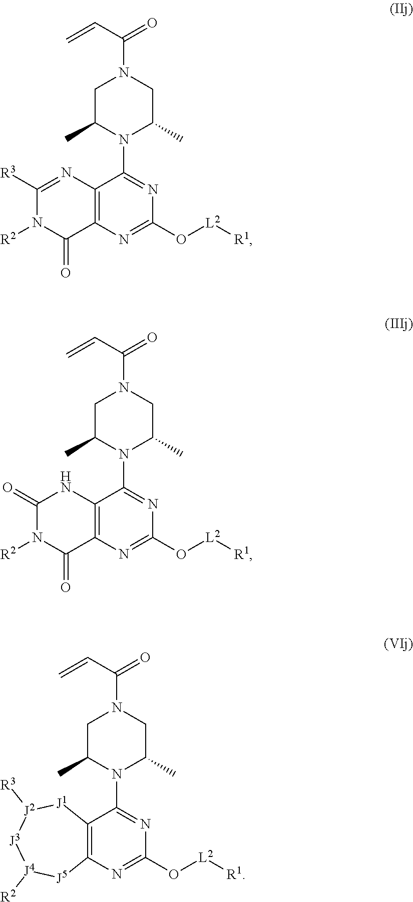

In some embodiments, the present disclosure provides a compound having a formula selected from the group consisting of:

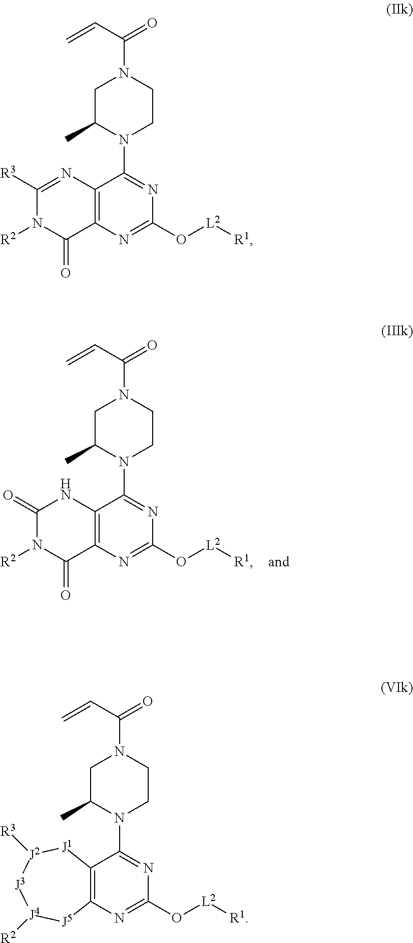

In some embodiments, L² is alkyl.

In some embodiments, R¹ is

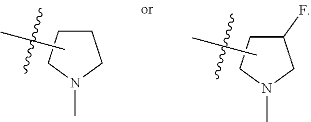

In some embodiments, R³ is selected from methyl, ethyl or trifluoromethyl.

In some embodiments, the present disclosure provides a compound having a formula selected from the group consisting of:

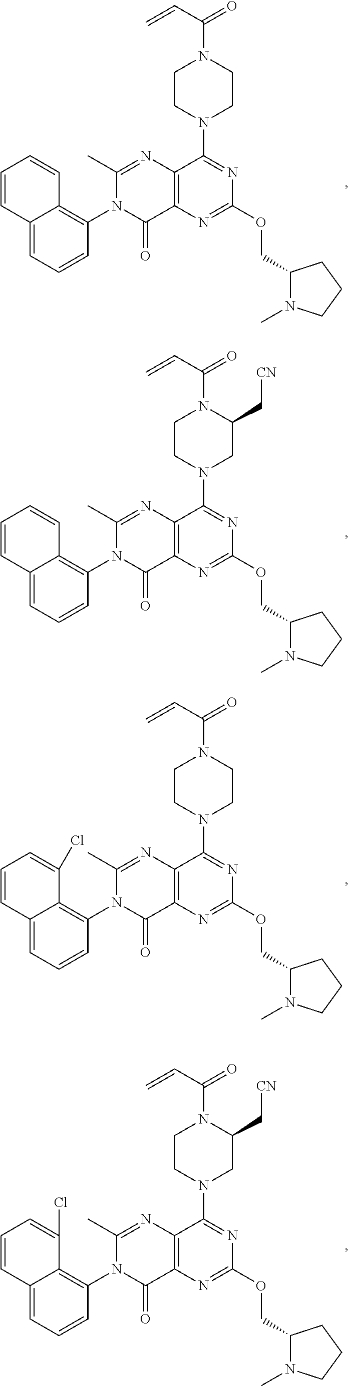

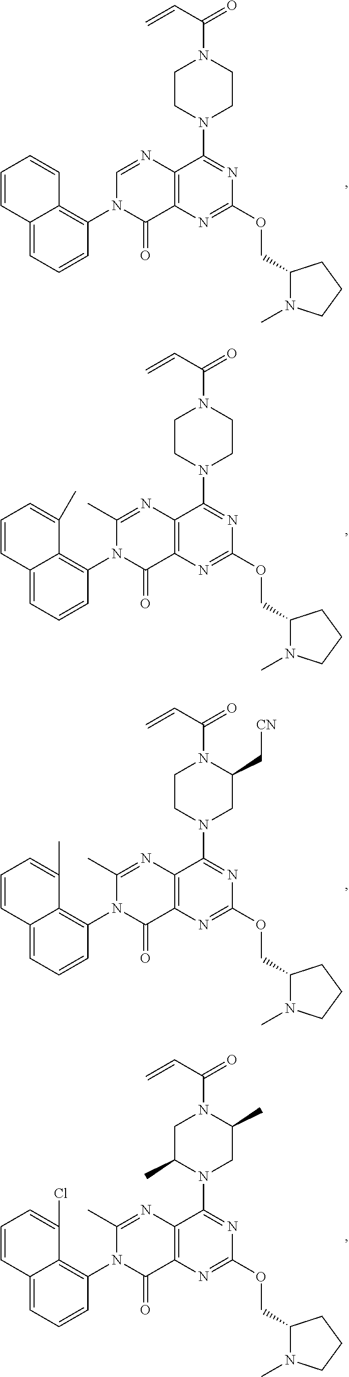

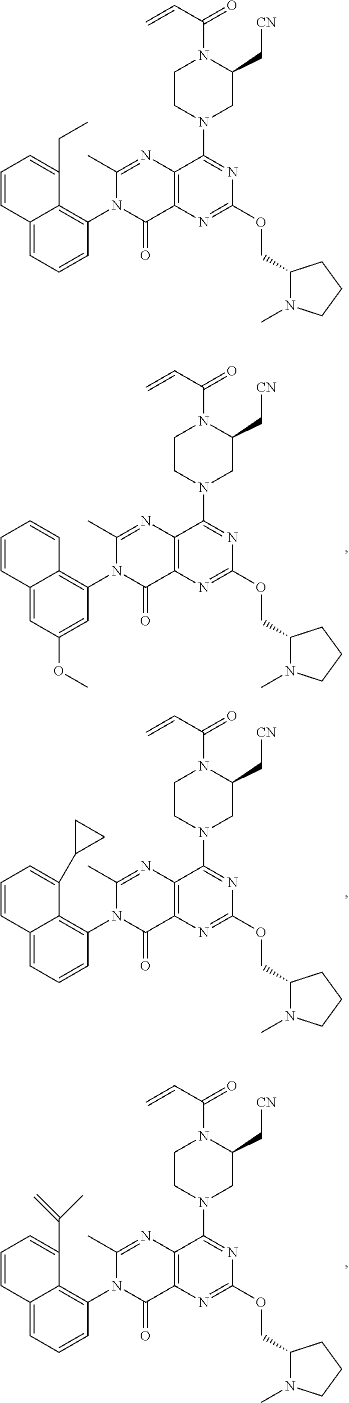

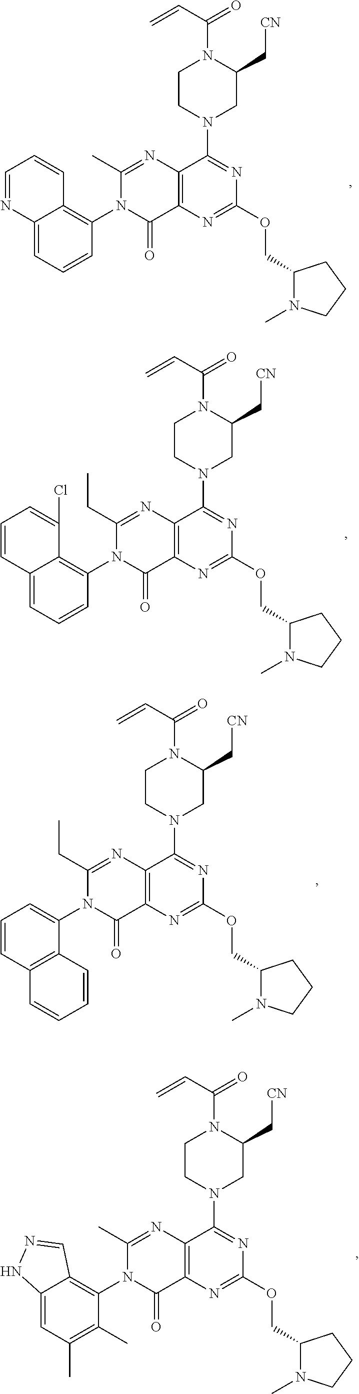 [SMILES: C=CC(=O)N1CCN(c2nc(OC[C@@H]3CCCN3C)nc3c(=O)n(-c4cccc5ncccc45)c(C)nc23)C[C@@H]1CC#N ; C=CC(=O)N1CCN(c2nc(OC[C@@H]3CCCN3C)nc3c(=O)n(-c4cccc5cccc(Cl)c45)c(CC)nc23)C[C@@H]1CC#N ; C=CC(=O)N1CCN(c2nc(OC[C@@H]3CCCN3C)nc3c(=O)n(-c4cccc5ccccc45)c(CC)nc23)C[C@@H]1CC#N ; C=CC(=O)N1CCN(c2nc(OC[C@@H]3CCCN3C)nc3c(=O)n(-c4c(C)c(C)cc5[nH]ncc45)c(C)nc23)C[C@@H]1CC#N]

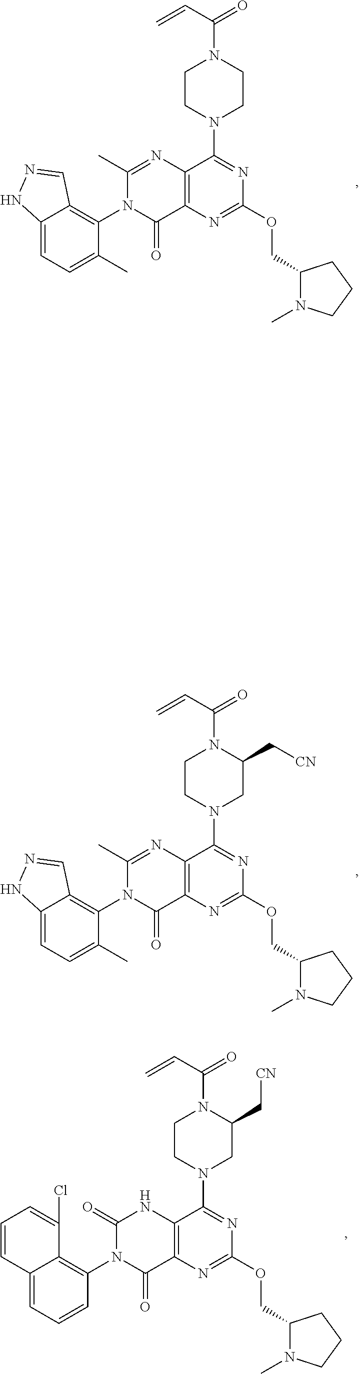 [SMILES: C=CC(=O)N1CCN(c2nc(OC[C@@H]3CCCN3C)nc3c(=O)n(-c4c(C)ccc5[nH]ncc45)c(C)nc23)CC1 ; C=CC(=O)N1CCN(c2nc(OC[C@@H]3CCCN3C)nc3c(=O)n(-c4c(C)ccc5[nH]ncc45)c(C)nc23)C[C@@H]1CC#N ; C=CC(=O)N1CCN(c2nc(OC[C@@H]3CCCN3C)nc3c(=O)n(-c4cccc5cccc(Cl)c45)c(=O)[nH]c23)C[C@@H]1CC#N]

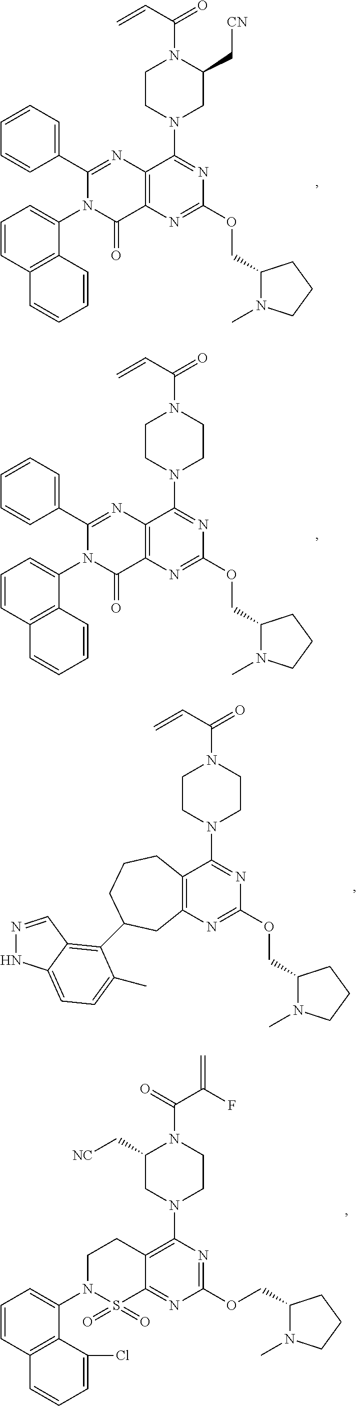

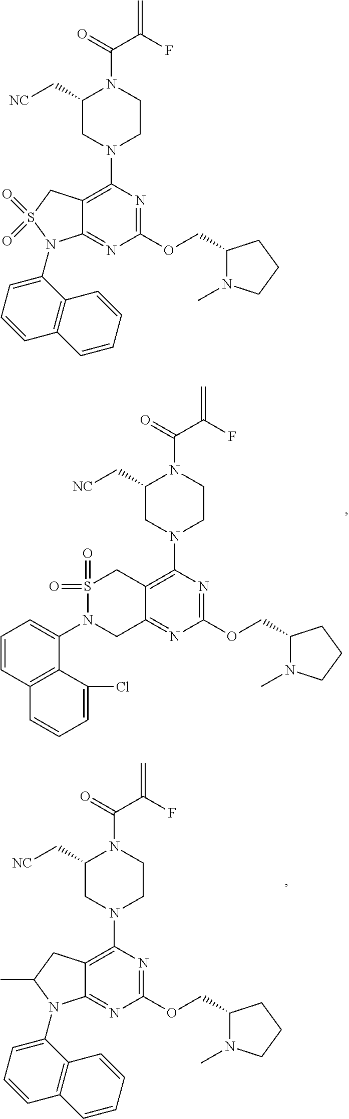 [SMILES: C=C(F)C(=O)N1CCN(c2nc(OC[C@@H]3CCCN3C)nc3c2CS(=O)(=O)N3c2cccc3ccccc23)C[C@@H]1CC#N ; C=C(F)C(=O)N1CCN(c2nc(OC[C@@H]3CCCN3C)nc3c2CS(=O)(=O)N(c2cccc4cccc(Cl)c24)C3)C[C@@H]1CC#N ; C=C(F)C(=O)N1CCN(c2nc(OC[C@@H]3CCCN3C)nc3c2CC(C)N3c2cccc3ccccc23)C[C@@H]1CC#N]

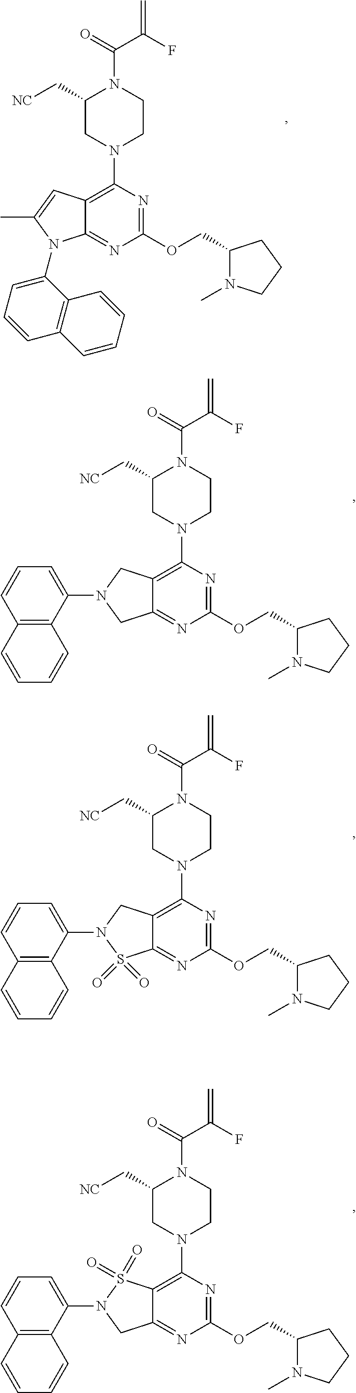 [SMILES: C=C(F)C(=O)N1CCN(c2nc(OC[C@@H]3CCCN3C)nc3c2cc(C)n3-c2cccc3ccccc23)C[C@@H]1CC#N ; C=C(F)C(=O)N1CCN(c2nc(OC[C@@H]3CCCN3C)nc3c2CN(c2cccc4ccccc24)C3)C[C@@H]1CC#N ; C=C(F)C(=O)N1CCN(c2nc(OC[C@@H]3CCCN3C)nc3c2CN(c2cccc4ccccc24)S3(=O)=O)C[C@@H]1CC#N ; C=C(F)C(=O)N1CCN(c2nc(OC[C@@H]3CCCN3C)nc3c2S(=O)(=O)N(c2cccc4ccccc24)C3)C[C@@H]1CC#N]

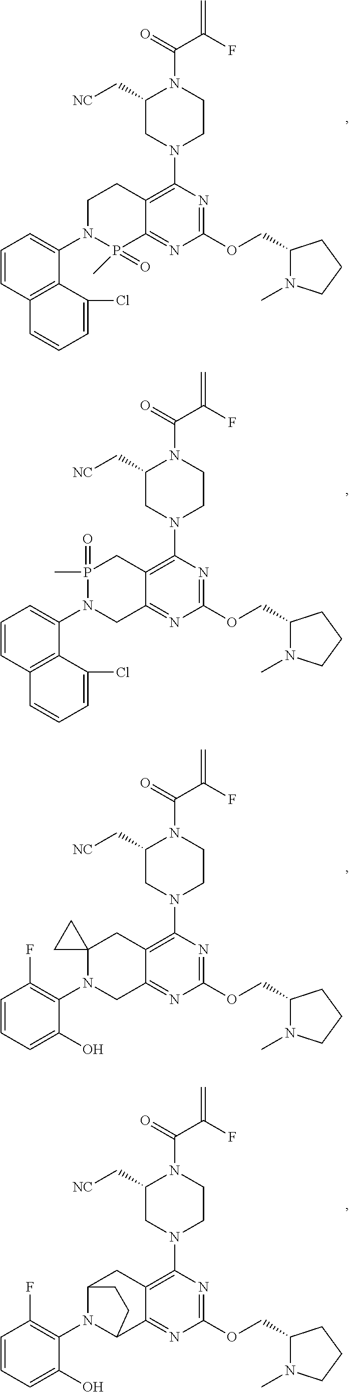 [SMILES: C=C(F)C(=O)N1CCN(c2nc(OC[C@@H]3CCCN3C)nc3c2CCN(c2cccc4cccc(Cl)c24)P3(C)=O)C[C@@H]1CC#N ; C=C(F)C(=O)N1CCN(c2nc(OC[C@@H]3CCCN3C)nc3c2CP(C)(=O)N(c2cccc4cccc(Cl)c24)C3)C[C@@H]1CC#N ; C=C(F)C(=O)N1CCN(c2nc(OC[C@@H]3CCCN3C)nc3c2CC2(CC2)N(c2c(O)cccc2F)C3)C[C@@H]1CC#N ; C=C(F)C(=O)N1CCN(c2nc(OC[C@@H]3CCCN3C)nc3c2CC2CCC3N2c2c(O)cccc2F)C[C@@H]1CC#N]

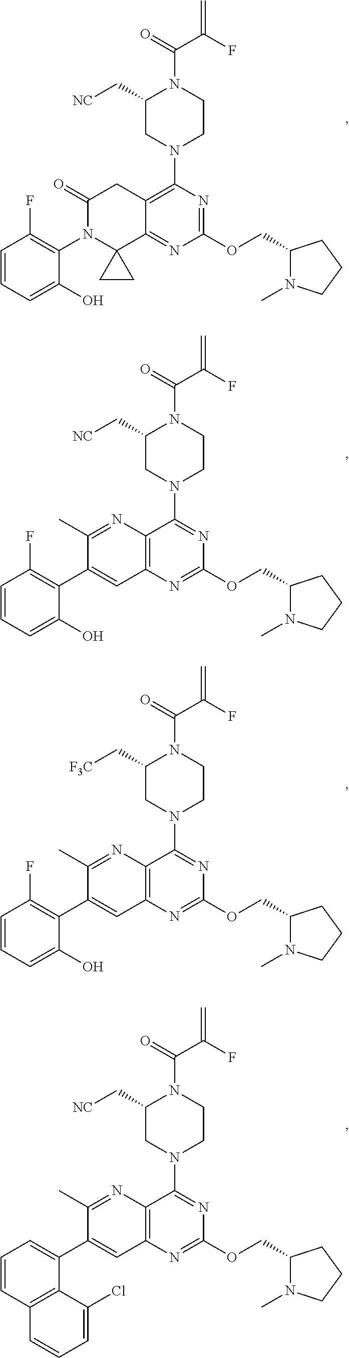 [SMILES: C=C(F)C(=O)N1CCN(c2nc(OC[C@@H]3CCCN3C)nc3c2CC(=O)N(c2c(O)cccc2F)C32CC2)C[C@@H]1CC#N ; C=C(F)C(=O)N1CCN(c2nc(OC[C@@H]3CCCN3C)nc3cc(-c4c(O)cccc4F)c(C)nc23)C[C@@H]1CC#N ; C=C(F)C(=O)N1CCN(c2nc(OC[C@@H]3CCCN3C)nc3cc(-c4c(O)cccc4F)c(C)nc23)C[C@@H]1CC(F)(F)F ; C=C(F)C(=O)N1CCN(c2nc(OC[C@@H]3CCCN3C)nc3cc(-c4cccc5cccc(Cl)c45)c(C)nc23)C[C@@H]1CC#N]

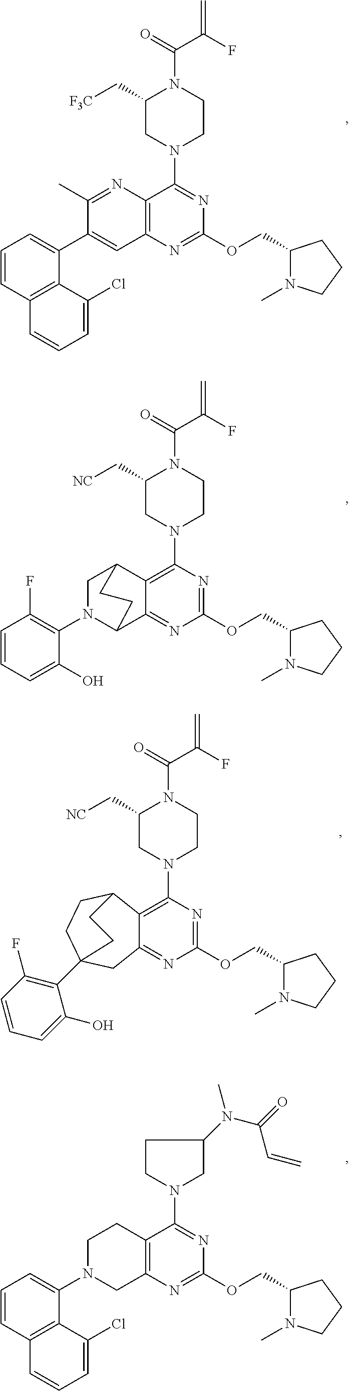 [SMILES: C=C(F)C(=O)N1CCN(c2nc(OC[C@@H]3CCCN3C)nc3cc(-c4cccc5cccc(Cl)c45)c(C)nc23)C[C@@H]1CC(F)(F)F ; C=C(F)C(=O)N1CCN(c2nc(OC[C@@H]3CCCN3C)nc3c2C2CCC3N(c3c(O)cccc3F)C2)C[C@@H]1CC#N ; C=C(F)C(=O)N1CCN(c2nc(OC[C@@H]3CCCN3C)nc3c2C2CCC(c4c(O)cccc4F)(CC2)C3)C[C@@H]1CC#N ; C=CC(=O)N(C)C1CCN(c2nc(OC[C@@H]3CCCN3C)nc3c2CCN(c2cccc4cccc(Cl)c24)C3)C1]

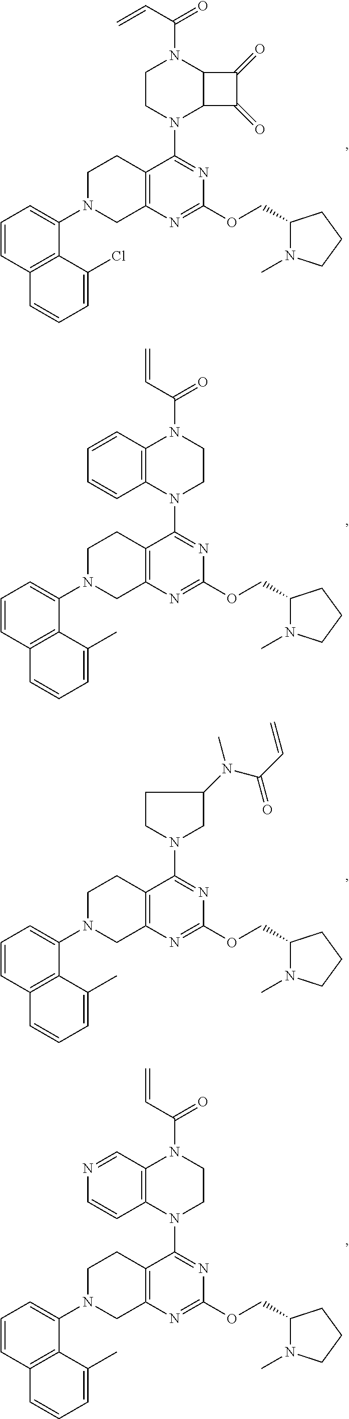 [SMILES: C=CC(=O)N1CCN(c2nc(OC[C@@H]3CCCN3C)nc3c2CCN(c2cccc4cccc(Cl)c24)C3)C2C(=O)C(=O)C21 ; C=CC(=O)N1CCN(c2nc(OC[C@@H]3CCCN3C)nc3c2CCN(c2cccc4cccc(C)c24)C3)c2ccccc21 ; C=CC(=O)N(C)C1CCN(c2nc(OC[C@@H]3CCCN3C)nc3c2CCN(c2cccc4cccc(C)c24)C3)C1 ; C=CC(=O)N1CCN(c2nc(OC[C@@H]3CCCN3C)nc3c2CCN(c2cccc4cccc(C)c24)C3)c2ccncc21]

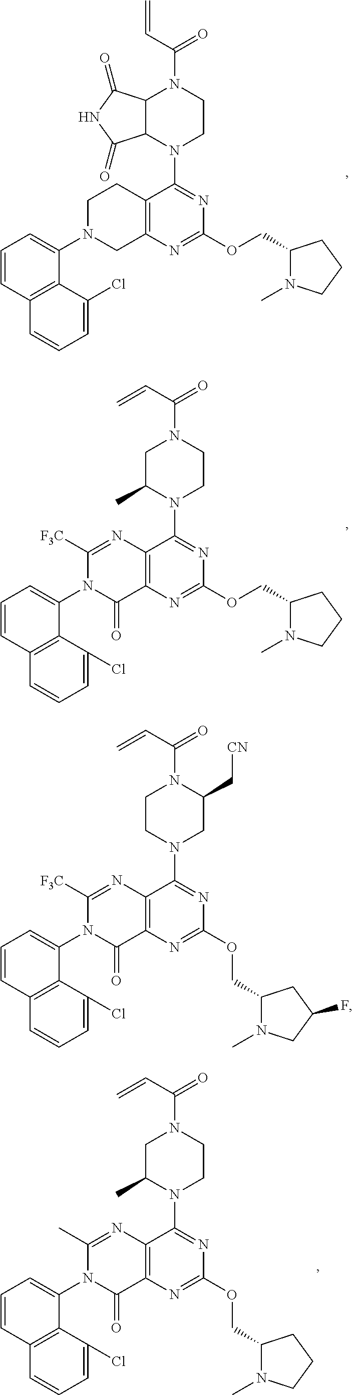

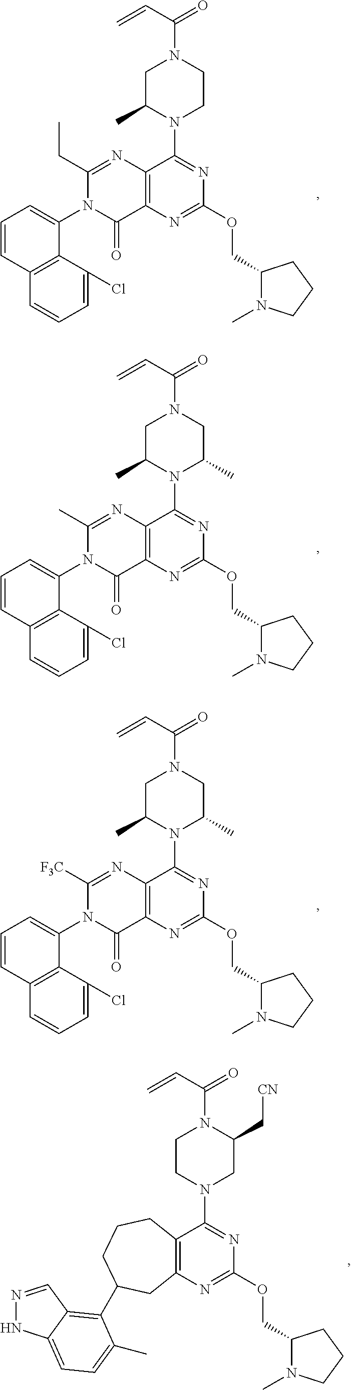

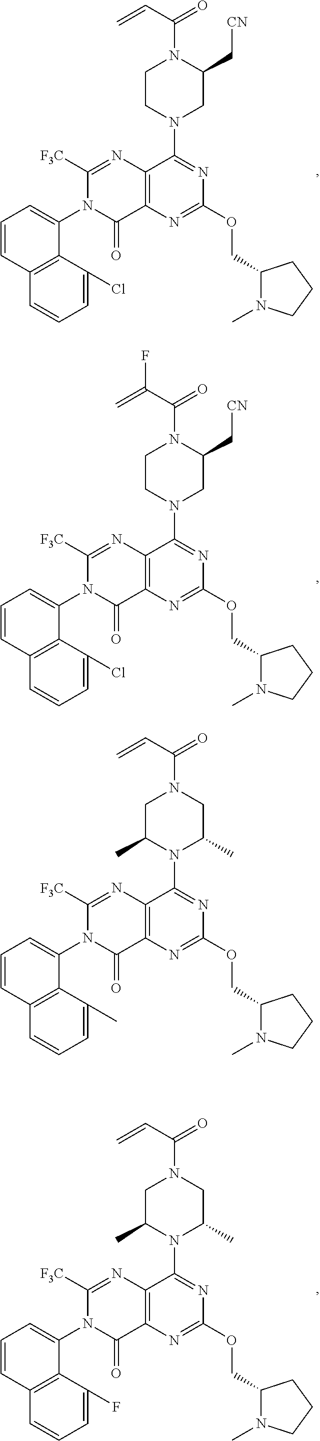

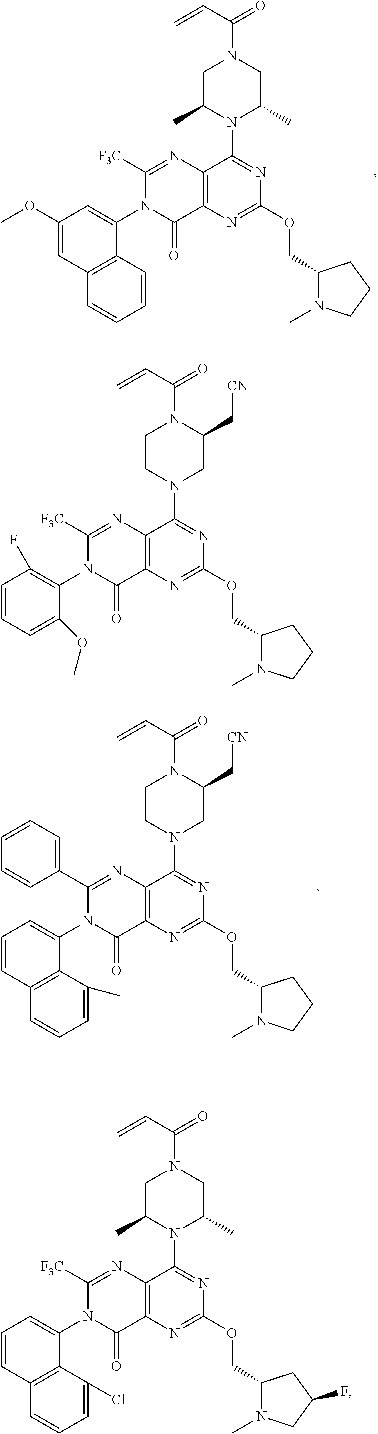

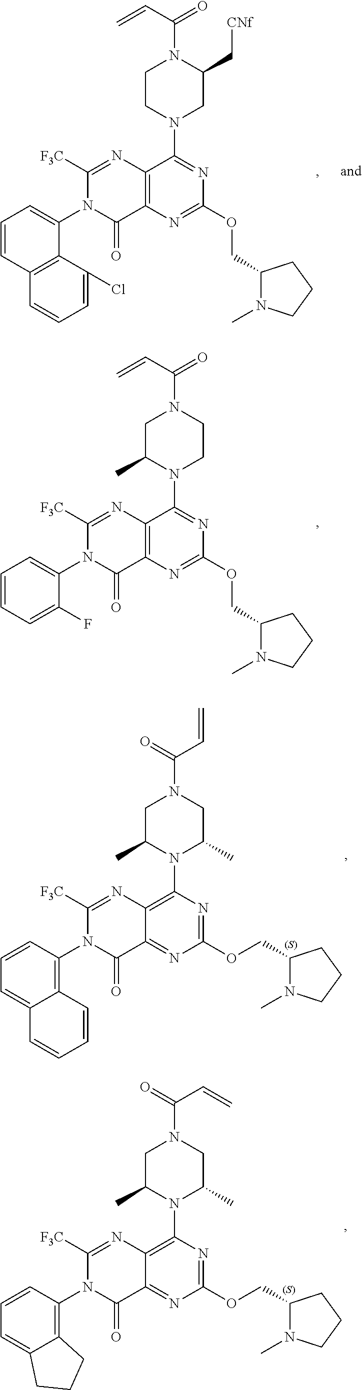

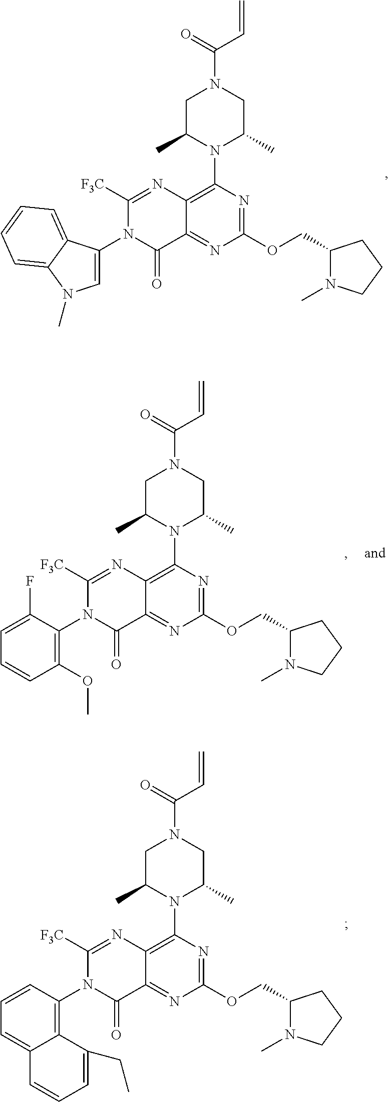 [SMILES: C=CC(=O)N1C[C@H](C)N(c2nc(OC[C@@H]3CCCN3C)nc3c(=O)n(-c4cn(C)c5ccccc45)c(C(F)(F)F)nc23)[C@@H](C)C1 ; C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1 ; C=CC(=O)N1C[C@H](C)N(c2nc(OC[C@@H]3CCCN3C)nc3c(=O)n(-c4cccc5cccc(CC)c45)c(C(F)(F)F)nc23)[C@@H](C)C1]

or a pharmaceutically acceptable salt thereof.

Compounds provided herein are described with reference to both generic formulae and specific compounds. In addition, the compounds of the present disclosure may exist in a number of different forms or derivatives, including but not limited to prodrugs, soft drugs, active metabolic derivatives (active metabolites), and their pharmaceutically acceptable salts, all within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

Those of skill in the art will appreciate that compounds of the present disclosure may exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples are characterized by nuclear magnetic resonance (NMR). NMR spectra were acquired on Bruker AVANCE III HD 400 nuclear magnetic resonance spectrometer, running at 400 MHz for ¹H and 101 MHz for ¹³C respectively. ¹H NMR spectra were recorded at 400 MHz in CHCl₃-d, (CH₃)₂SO-d₆ and (CH₃)₂CO-d₆ using residual CHCl₃ (7.26 ppm), DMSO (2.50 ppm) and (CH₃)₂CO (2.05 ppm) as the internal standard. ¹³C NMR spectra were recorded at 101 MHz in CHCl₃-d, (CH₃)₂SO-d₆ and (CH₃)₂CO-d₆ using residual CHCl₃ (77.16 ppm), DMSO (39.52 ppm) and (CH₃)₂CO (29.84 ppm and 206.26 ppm), as internal reference.

Mass spectrometry was performed at the mass spectrometry facility of School of Pharmaceutical Sciences at Tsinghua University on a Thermo Scientific QExactive mass spectrometer (ESI).

Thin layer chromatography was performed on Merck Kieselgel 60 Å F254 plates eluting with the solvent indicated, visualized by a 254 nm UV lamp, and stained with an ethanolic solution of 12-molybdophosphoric acid. Compounds were purified using flash chromatography (Silica gel 60 Å, 230-400 mesh, Silicycle Inc.).

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

For illustrative purposes, the Examples section below shows synthetic route for preparing the compounds of the present disclosure as well as key intermediates. Those skilled in the art will appreciate that other synthetic routes may be used to synthesize the inventive compounds. Although specific starting materials and reagents are depicted, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Use of Compounds

In an aspect, the present disclosure provides compounds of Formula (I) or pharmaceutically acceptable salts thereof, which are capable of inhibiting KRAS protein, in particular KRAS G12C protein.

As used herein, the term “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Therapy” can also mean prolonging survival as compared to expected survival if not receiving it. Those in need of therapy include those already with the condition or disorder as well as those prone to have the condition or disorder or those in which the condition or disorder is to be prevented. The term “therapy” also encompasses prophylaxis unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In a further aspect, the present disclosure provides use of the compound of the present disclosure or a pharmaceutically acceptable salt thereof or the pharmaceutical composition of the present disclosure for use in therapy, for example, for use in therapy associated with KRAS protein.

In a further aspect, the present disclosure provides use of the compound of the present disclosure or a pharmaceutically acceptable salt thereof or the pharmaceutical composition of the present disclosure, in the manufacture of a medicament for treating cancer.

In some embodiments, the cancer is mediated by KRAS protein. In some embodiments, the cancer is mediated by KRAS-G12C mutant protein.

In a further aspect, the present disclosure provides use of the compound of the present disclosure or a pharmaceutically acceptable salt thereof or the pharmaceutical composition of the present disclosure, in the manufacture of a medicament for inhibiting tumor metastasis.

In another aspect, the present disclosure provides a compound of the present disclosure or a pharmaceutically acceptable salt thereof or a pharmaceutical composition of the present disclosure, for treating cancer.

In another aspect, the present disclosure provides a compound of the present disclosure or a pharmaceutically acceptable salt thereof or a pharmaceutical composition of the present disclosure, for inhibiting tumor metastasis.

### Pharmaceutical Compositions

In a further aspect, there is provided pharmaceutical compositions comprising one or more molecules or compounds of the present disclosure, or a pharmaceutically acceptable salt thereof.

In another aspect, there is provided pharmaceutical composition comprising one or more molecules or compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

In some embodiments, the second active ingredient has complementary activities to the compound provided herein such that they do not adversely affect each other. Such ingredients are suitably present in combination in amounts that are effective for the purpose intended.

### Method of Treatment of Disease

In a further aspect, the present disclosure provides a method for treating cancer, comprising administering an effective amount of the compound or a pharmaceutically acceptable salt thereof or the pharmaceutical composition provided herein to a subject in need thereof.

In some embodiments, said method relates to the treatment of cancer such as lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, hematological cancer, colorectal cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, MYH associated polyposis, or pituitary adenoma.

In some embodiments, the cancer is associated with KRAS G12C mutation. In certain embodiments, the cancer is a hematological cancer, pancreatic cancer, MYH associated polyposis, colorectal cancer, or lung cancer.

In another aspect, the present disclosure also provides a method for treating cancer in a subject in need thereof, the method comprising:
(a) determining that the cancer is associated with KRAS G12C mutation; and
(b) administering to the subject an effective amount of a compound or a pharmaceutically acceptable salt thereof or the pharmaceutical composition of the present disclosure.

In a further aspect, the present disclosure provides a method for inhibiting tumor metastasis, comprising administering an effective amount of a compound or a pharmaceutically acceptable salt thereof or the pharmaceutical composition of the present disclosure to a subject in need thereof.

In another aspect, the present disclosure provides a method for regulating activity of a KRAS G12C mutant protein, comprising reacting the KRAS G12C mutant protein with the compound or a pharmaceutically acceptable salt thereof or the pharmaceutical composition of the present disclosure.

In a further aspect, the present disclosure provides a method for preparing a labeled KRAS G12C mutant protein, comprising reacting the KRAS G12C mutant protein with the compound or a pharmaceutically acceptable salt thereof provided herein, to result in the labeled KRAS G12C mutant protein.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1

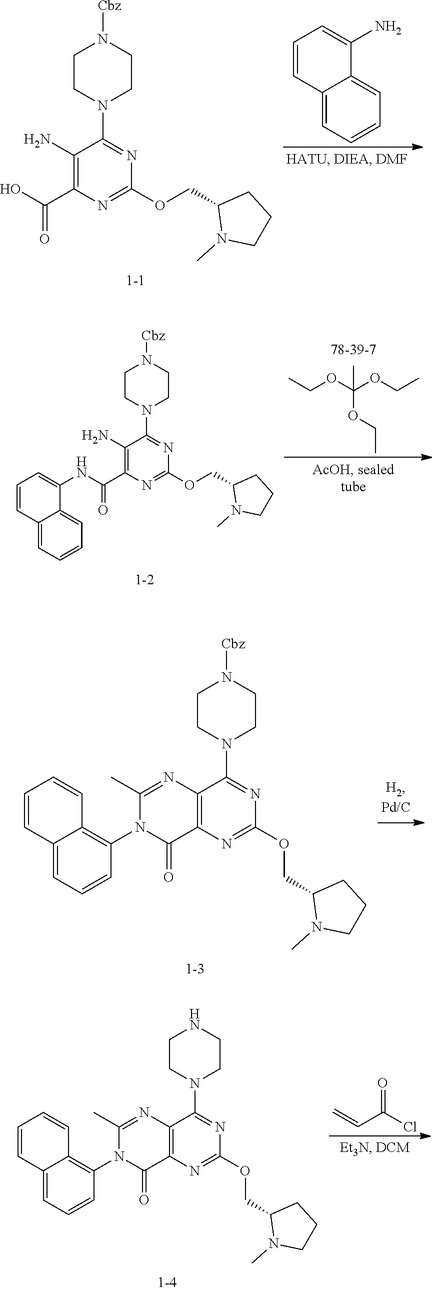

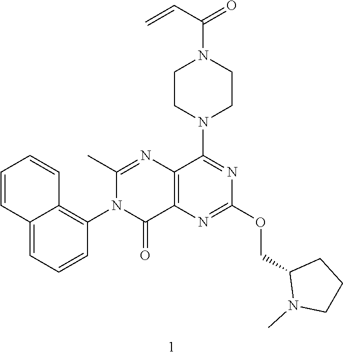 [SMILES: C=CC(=O)N1CCN(c2nc(OC[C@@H]3CCCN3C)nc3c(=O)n(-c4cccc5ccccc45)c(C)nc23)CC1]

### Step 1: Synthesis of Compound 1-2

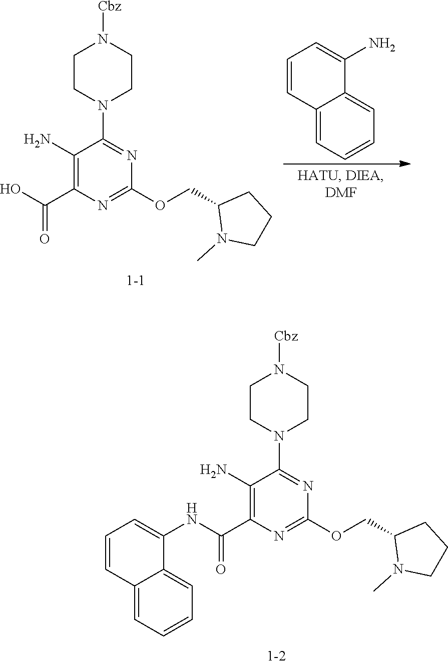 [SMILES: CN1CCC[C@H]1COc1nc(C(=O)O)c(N)c(N2CCN(C(=O)OCc3ccccc3)CC2)n1 ; Nc1cccc2ccccc12 ; CN1CCC[C@H]1COc1nc(C(=O)Nc2cccc3ccccc23)c(N)c(N2CCN(C(=O)OCc3ccccc3)CC2)n1]

To a mixture of 5-amino-6-((S)-4-((benzyloxy)carbonyl)-3-(cyanomethyl)piperazin-1l-yl)-2-(((S)-1l-methylpyrrolidin-2-yl)methoxy)pyrimidine-4-carboxylic acid (270 mg, 0.574 mmol, 1.0 eq.) and naphthalen-1-amine (82 mg, 0.574 mmol, 1.0 eq.) in anhydrous DMF (3.0 mL) was added DIEA (0.28 mL, 1.722 mmol, 3.0 eq.), followed by the addition of HATU (435 mg, 1.144 mmol, 2.0 eq.). The reaction mixture was stirred at 60° C. under Ar for 1 h. LCMS showed starting material was consumed and desired product formed. The reaction mixture was cooled to room temperature, diluted with EtOAc (30 mL) and washed with brine (3×30 mL), dried over anhydrous Na₂SO₄, filtered and concentrated under reduced pressure. The residue was purified by silica column chromatography eluting with DCM/MeOH (1/0-10:1, v/v) to obtain benzyl (S)-4-(5-amino-2-((1-methylpyrrolidin-2-yl)methoxy)-6-(naphthalen-1-ylcarbamoyl)pyrimidin-4-yl)piperazine-1-carboxylate (80 mg, 17%).

LCMS: Rt: 0.941 min; MS m/z (ESI): 596.3 [M+H]⁺.

### Step 2: Synthesis of Compound 1-3

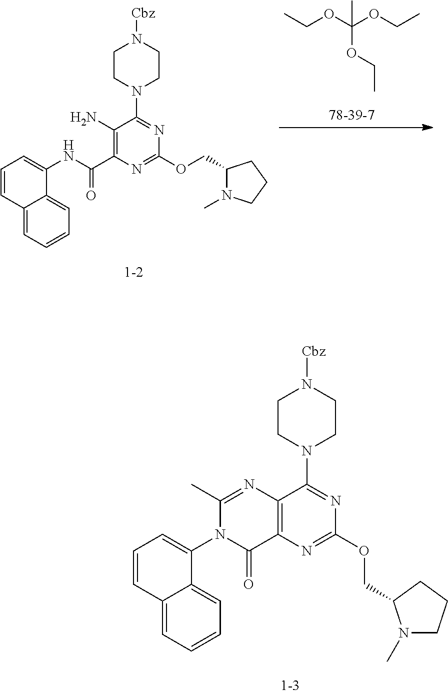 [SMILES: CN1CCC[C@H]1COc1nc(C(=O)Nc2cccc3ccccc23)c(N)c(N2CCN(C(=O)OCc3ccccc3)CC2)n1 ; CCOC(C)(OCC)OCC ; Cc1nc2c(N3CCN(C(=O)OCc4ccccc4)CC3)nc(OC[C@@H]3CCCN3C)nc2c(=O)n1-c1cccc2ccccc12]

To a mixture of benzyl (S)-4-(5-amino-2-((1-methylpyrrolidin-2-yl)methoxy)-6-(naphthalen-1-ylcarbamoyl)pyrimidin-4-yl)piperazine-1-carboxylate (60 mg, 0.0504 mmol, 1.0 eq.) in EtOH (1 mL) was added 1,1,1-triethoxyethane (1 mL) and AcOH (6 drops). The mixture was stirred at 145° C. in a sealed tube for 2 h. LCMS showed starting material was consumed and desired product formed. The reaction mixture was adjusted to pH=8-9 with aq. NaHCO₃ solution and extracted with DCM (3×20 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-4-(6-methyl-2-((1-methylpyrrolidin-2-yl)methoxy)-7-(naphthalen-1-yl)-8-oxo-7,8-dihydropyrimido[5,4-d]pyrimidin-4-yl)piperazine-1-carboxylate (15 mg, 18%).

LCMS: Rt: 0.966 min; MS m/z (ESI): 620.3 [M+H]⁺.

### Step 3: Synthesis of Compound 1-4

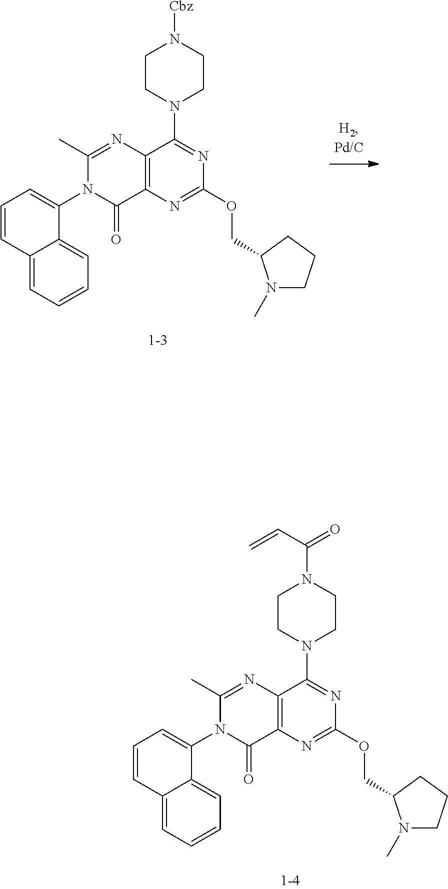 [SMILES: Cc1nc2c(N3CCN(C(=O)OCc4ccccc4)CC3)nc(OC[C@@H]3CCCN3C)nc2c(=O)n1-c1cccc2ccccc12 ; C=CC(=O)N1CCN(c2nc(OC[C@@H]3CCCN3C)nc3c(=O)n(-c4cccc5ccccc45)c(C)nc23)CC1]

To a mixture of benzyl (S)-4-(6-methyl-2-((1-methylpyrrolidin-2-yl)methoxy)-7-(naphthalen-1-yl)-8-oxo-7,8-dihydropyrimido[5,4-d]pyrimidin-4-yl)piperazine-1-carboxylate (15 mg, 0.024 mmol) in MeOH (5.0 mL) was Pd(OH)₂/C (10 mg, 20% wt), and the mixture was stirred at room temperature for 2 h under H₂ (50 psi). LCMS showed starting material was consumed and desired product formed. The resulting mixture was filtered through celite. The filter cake was washed with MeOH (30 mL). The filtrate was concentrated under reduced pressure to obtain (S)-2-methyl-6-((1-methylpyrrolidin-2-yl)methoxy)-3-(naphthalen-1-yl)-8-(piperazin-1-yl)pyrimido[5,4-d]pyrimidin-4(3H)-one (12 mg, 100%) as a yellow solid, which was used directly for the next step without further purification.

LCMS: Rt: 0.557 min; MS m/z (ESI): 486.2 [M+H]⁺.

### Step 4: Synthesis of Compound 1

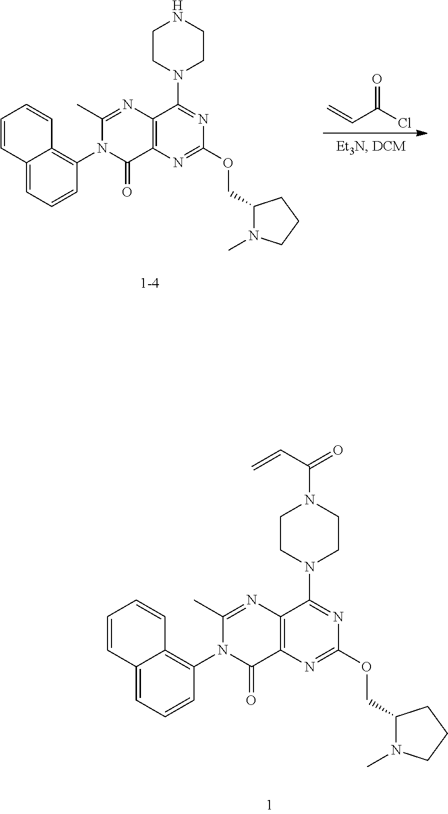

To a mixture of (S)-2-methyl-6-((1-methylpyrrolidin-2-yl)methoxy)-3-(naphthalen-1-yl)-8-(piperazin-1-yl)pyrimido[5,4-d]pyrimidin-4(3H)-one (12 mg, 0.024 mmol, 1.0 eq.) and Et₃N (7 mg, 0.072 mmol, 3.0 eq.) in DCM (1 mL) was added dropwise a solution of acryloyl chloride (2.2 mg, 0.024 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by basic prep-HPLC separation to obtain (S)-8-(4-acryloylpiperazin-1-yl)-2-methyl-6-((1-methylpyrrolidin-2-yl)methoxy)-3-(naphthalen-1-yl)pyrimido[5,4-d]pyrimidin-4(3H)-one (3.5 mg, 27%, 1).

LCMS: Rt: 0.839 min; MS m/z (ESI): 540.3 [M+H]⁺;

¹H NMR (400 MHz, CDCl3) δ 8.04 (d, J=8.2 Hz, 1H), 7.98 (d, J=8.2 Hz, 1H), 7.65-7.50 (m, 3H), 7.47-7.40 (m, 2H), 6.68-6.58 (m, 1H), 6.42-6.34 (m, 1H), 5.83-5.73 (m, 1H), 5.12-4.75 (m, 1H), 4.70-4.22 (m, 4H), 3.95-3.70 m, 6H), 3.17-2.82 (m, 4H), 2.42-2.13 (m, 3H), 2.11 (s, 3H), 1.41-1.22 (m, 2H).

### Example 2

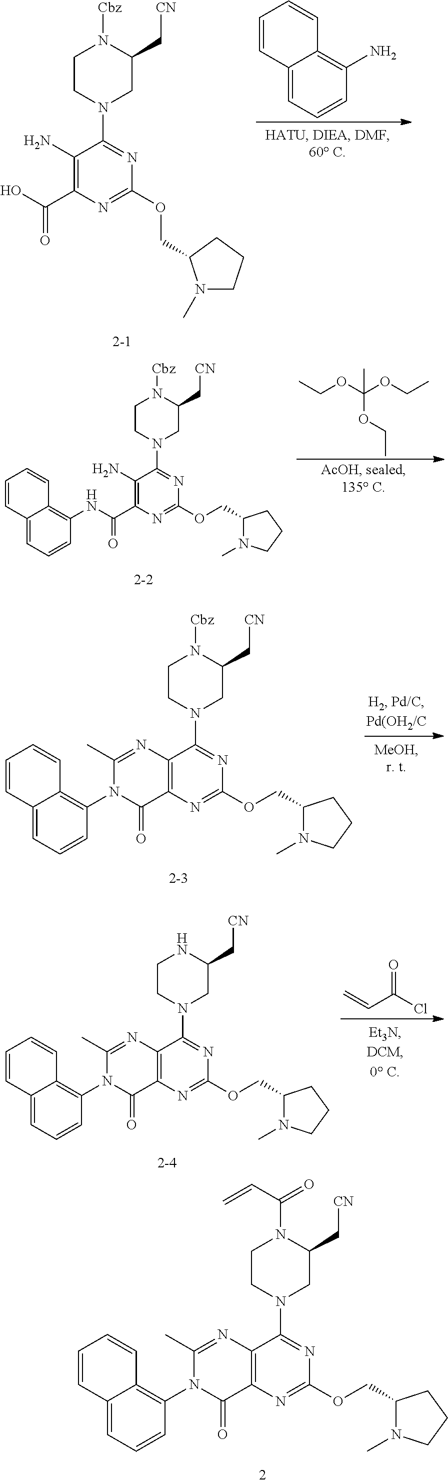

Step 1: Synthesis of Compound 2-2

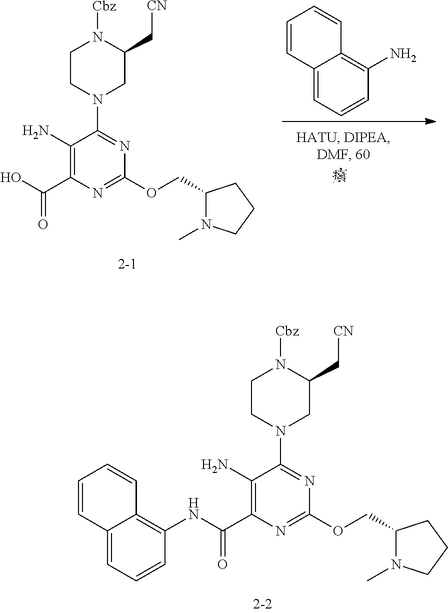 [SMILES: CN1CCC[C@H]1COc1nc(C(=O)O)c(N)c(N2CCN(C(=O)OCc3ccccc3)[C@@H](CC#N)C2)n1 ; Nc1cccc2ccccc12 ; CN1CCC[C@H]1COc1nc(C(=O)Nc2cccc3ccccc23)c(N)c(N2CCN(C(=O)OCc3ccccc3)[C@@H](CC#N)C2)n1]

To a solution of 5-amino-6-((S-4-((benzyloxy)carbonyl)-3-(cyanomethyl)piperazin-1-yl)-2-(((S)-1-methylpyrrolidin-2-yl)methoxy)pyrimidine-4-carboxylic acid (300 mg, 0.589 mmol, 1.0 eq.) and naphthalen-1-amine (59 mg, 0.412 mmol, 0.7 eq.) in anhydrous DMF (4.0 mL) was added DIEA (0.29 mL, 1.77 mmol, 3.0 eq.), followed by the addition of HATU (224 mg, 0.589 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (20 mL×2). The combined organic fractions were washed with brine (30 mL), dried over anhydrous Na₂SO₄ and concentrated. The residue was purified by silica column chromatography eluting with DCM/MeOH (20:1, v/v) to obtain benzyl (S)-4-(5-amino-2-(((S)-1-methylpyrrolidin-2-yl)methoxy)-6-(naphthalen-1-ylcarbamoyl) pyrimidin-4-yl)-2-(cyanomethyl)piperazine-1-carboxylate (223 mg, 60%).

LCMS (ESI, m/z): [M+1]⁺=635; RT=1.242 min.

### Step 2: Synthesis of Compound 2-3

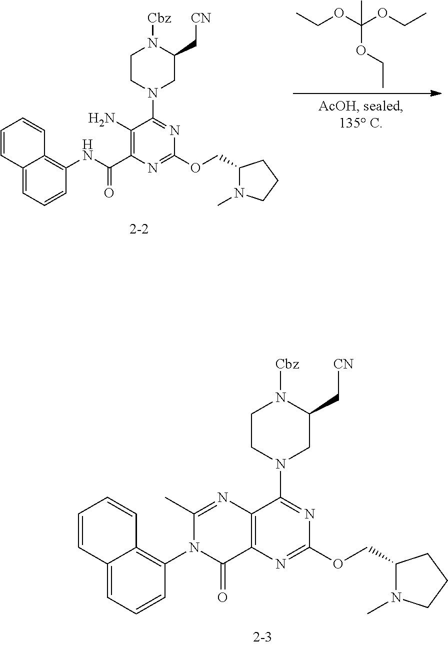 [SMILES: CN1CCC[C@H]1COc1nc(C(=O)Nc2cccc3ccccc23)c(N)c(N2CCN(C(=O)OCc3ccccc3)[C@@H](CC#N)C2)n1 ; CCOC(C)(OCC)OCC ; Cc1nc2c(N3CCN(C(=O)OCc4ccccc4)[C@@H](CC#N)C3)nc(OC[C@@H]3CCCN3C)nc2c(=O)n1-c1cccc2ccccc12]

To a mixture of (S)-4-(5-amino-2-(((S)-1-methylpyrrolidin-2-yl)methoxy)-6-(naphthalen-1-ylcarbamoyl) pyrimidin-4-yl)-2-(cyanomethyl)piperazine-1-carboxylate (90 mg, 0.071 mmol, 1.0 eq.) and AcOH (1.0 mL) was added 1,1,1-triethoxyethane (346 mg, 1.06 mmol, 15.0 eq.). The mixture was stirred at 135° C. in a sealed tube for 7 min. LCMS showed starting material was consumed and desired product formed. The reaction mixture was quenched with aq. NaHCO₃ (sat. 60 mL) to adjust pH=7-8, which was extracted with DCM (20 mL×3). The combined organic fractions were dried over anhydrous Na₂SO₄ and concentrated. The residue was purified by silica column chromatography eluting with DCM/MeOH (15:1, v/v) to obtain benzyl (S)-2-(cyanomethyl)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (43 mg, 46%).

LCMS (ESI, m/z): [M+1]⁺=659; RT=1.194 min.

### Step 3: Synthesis of Compound 2-4

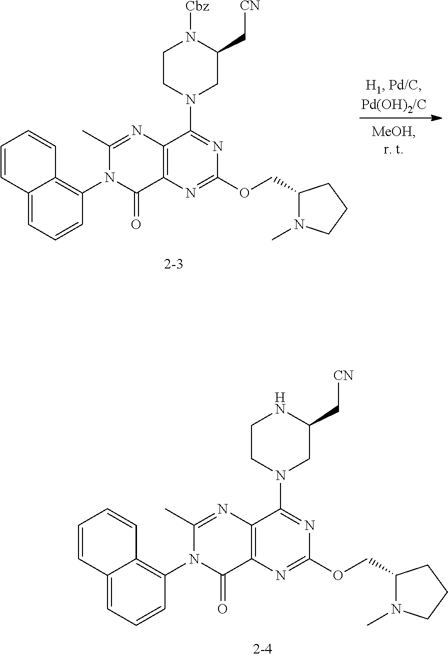 [SMILES: Cc1nc2c(N3CCN(C(=O)OCc4ccccc4)[C@@H](CC#N)C3)nc(OC[C@@H]3CCCN3C)nc2c(=O)n1-c1cccc2ccccc12 ; Cc1nc2c(N3CCN[C@@H](CC#N)C3)nc(OC[C@@H]3CCCN3C)nc2c(=O)n1-c1cccc2ccccc12]

To a solution of benzyl (S)-2-(cyanomethyl)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (43 mg, 0.065 mmol, 1.0 eq.) in i-PrOH (1.5 mL) and THF (1.5 mL) was added Pd/C (10% w/w, 7 mg, 0.0065 mmol, 0.1 eq.) and Pd(OH)₂/C (10% w/w, 9 mg, 0.0065 mmol, 0.1 eq.). The reaction mixture was stirred at room temperature under H₂ (balloon) for 2 h. LCMS showed most of starting material was consumed and desired product formed. The mixture was filtered through celite and the filtrate was concentrated to dryness to obtain 2-((S)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (18 mg, 53%) as light yellow solid, which was used directly for the next step.

LCMS (ESI, m/z): [M+1]⁺=525; RT=0.381 min & 0.565 min.

### Step 4: Synthesis of Compound 2

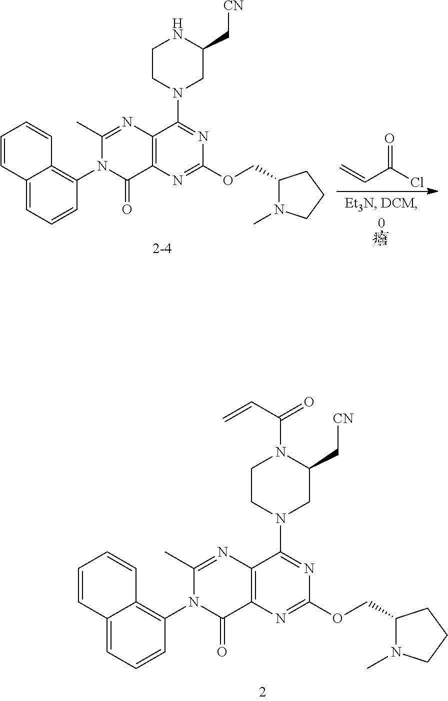

To a cooled (0° C.) solution of 2-((S)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (18 mg, 0.034 mmol, 1.0 eq.) and Et₃N (10.4 mg, 0.103 mmol, 3.0 eq.) in DCM (1.5 mL) was added dropwise a solution of acryloyl chloride (3.1 mg, 0.034 mmol, 1.0 eq.) in DCM (0.3 mL). After addition, the mixture was stirred at 0° C. for 15 min. LCMS showed most of starting material was consumed and desired product formed. Water (10 mL) was added and the organic layer was separated. The aqueous layer was extracted with DCM (5 mL×3). The combined organic fractions were dried over anhydrous Na₂SO₄ and concentrated. The residue was purified by prep-HPLC (ACN-H₂O+0.1% HCOOH) to obtain 2-((S)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (HCOOH salt, 2.1 mg, 9.9%, 2·0.63HCOOH) (C₃₂H₃₄N₈O₃·0.63HCOOH).

LCMS (ESI, m/z): [M+1]⁺=579; RT=0.994 min.

¹H NMR (400 MHz, DMSO-d₆) δ 8.33 (s, 1H), 8.32 (s, 0.63H), 8.13 (dd, J=13.4, 8.2 Hz, 2H), 7.76-7.50 (m, 4H), 6.95-6.81 (m, 1H), 6.20 (dd, J=16.8, 2.0 Hz, 1H), 5.79 (d, J=10.8 Hz, 1H), 5.59-5.36 (m, 1H), 5.10-4.77 (m, 2H), 4.49 (d, J=7.6 Hz, 1H), 4.38-4.28 (m, 1H), 4.18-4.10 (m, 1H), 3.33-3.12 (m, 4H), 3.02-2.92 (m, 2H), 2.65-2.52 (m, 1H), 2.37-2.28 (m, 4H), 2.18 (dd, J=17.0, 8.6 Hz, 1H), 2.06-1.84 (m, 4H), 1.73-1.55 (m, 3H).

### Example 3

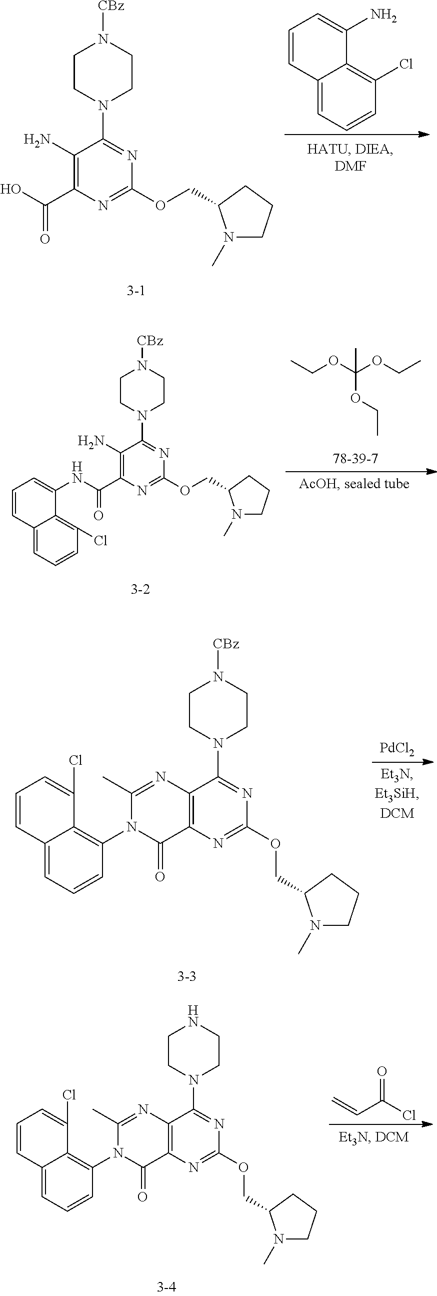

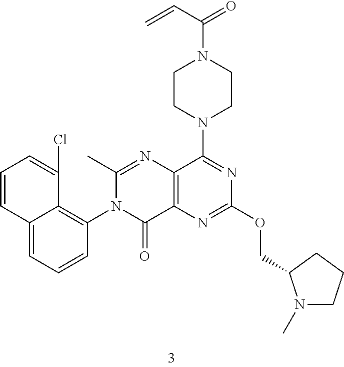 [SMILES: C=CC(=O)N1CCN(c2nc(OC[C@@H]3CCCN3C)nc3c(=O)n(-c4cccc5cccc(Cl)c45)c(C)nc23)CC1]

### Step 1: Synthesis of Compound 3-2

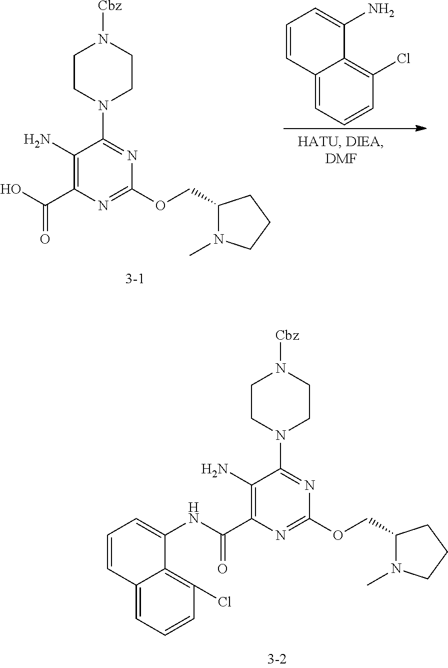 [SMILES: CN1CCC[C@H]1COc1nc(C(=O)O)c(N)c(N2CCN(C(=O)OCc3ccccc3)CC2)n1 ; Nc1cccc2cccc(Cl)c12 ; CN1CCC[C@H]1COc1nc(C(=O)Nc2cccc3cccc(Cl)c23)c(N)c(N2CCN(C(=O)OCc3ccccc3)CC2)n1]

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (120 mg, 0.255 mmol, 1.0 eq.) and 8-chloronaphthalen-1-amine (68 mg, 0.383 mmol, 1.5 eq.) in anhydrous DMF (3.0 mL) was added DIEA (99 mg, 0.765 mmol, 3.0 eq.), followed by the addition of HATU (194 mg, 0.51 mmol, 2.0 eq.). The reaction mixture was stirred at 60° C. under Ar for 1 h. LCMS showed the reaction was completed. The reaction mixture was cooled to room temperature, diluted with EtOAc (30 mL) and washed with brine (3×30 mL), dried over anhydrous Na₂SO₄, filtered and concentrated under reduced pressure. The residue was purified by preparative TLC with DCM/MeOH (10:1, v/v) to obtain benzyl (S)-4-(5-amino-6-((8-chloronaphthalen-1-yl)carbamoyl)-2-((1-methylpyrrolidin-2-yl)methoxy)pyrimidin-4-yl)piperazine-1-carboxylate (80 mg, 50%).

LCMS: Rt: 0.951 min; MS m/z (ESI): 630.2 [M+H]⁺.

### Step 2: Synthesis of Compound 3-3

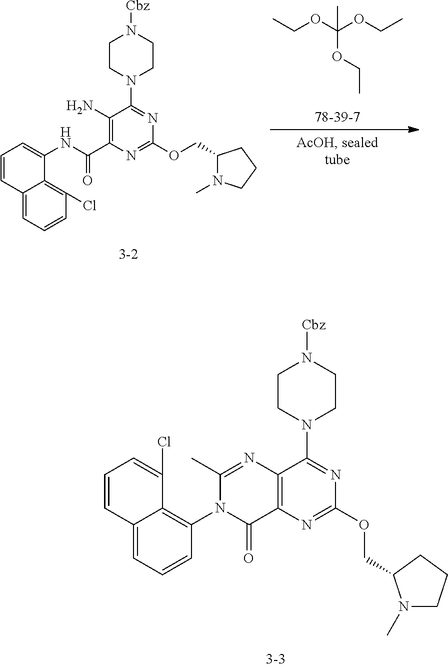 [SMILES: CN1CCC[C@H]1COc1nc(C(=O)Nc2cccc3cccc(Cl)c23)c(N)c(N2CCN(C(=O)OCc3ccccc3)CC2)n1 ; CCOC(C)(OCC)OCC ; Cc1nc2c(N3CCN(C(=O)OCc4ccccc4)CC3)nc(OC[C@@H]3CCCN3C)nc2c(=O)n1-c1cccc2cccc(Cl)c12]

To a mixture of benzyl (S)-4-(5-amino-6-((8-chloronaphthalen-1-yl)carbamoyl)-2-((1-methylpyrrolidin-2-yl)methoxy)pyrimidin-4-yl)piperazine-1-carboxylate (30 mg, 0.0477 mmol, 1.0 eq.) in AcOH (0.5 mL) was added 1,1,1-triethoxyethane (116 mg, 0.715 mmol, 15.0 eq.). The mixture was stirred at 135° C. in a sealed tube for 15 min. LCMS showed starting material was consumed and desired product formed. The reaction mixture was adjusted to pH=8-9 with aq. NaHCO₃ solution and extracted with DCM (3×15 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-4-(7-(8-chloronaphthalen-1-yl)-6-methyl-2-((1-methylpyrrolidin-2-yl)methoxy)-8-oxo-7,8-dihydropyrimido[5,4-d]pyrimidin-4-yl)piperazine-1-carboxylate (32 mg, 100%).

LCMS: Rt: 0.969 min; MS m/z (ESI): 654.3 [M+H]⁺.

### Step 3: Synthesis of Compound 3-4

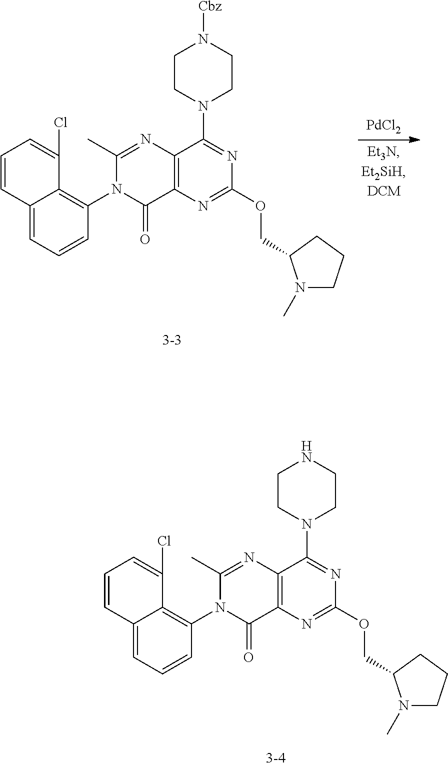 [SMILES: Cc1nc2c(N3CCN(C(=O)OCc4ccccc4)CC3)nc(OC[C@@H]3CCCN3C)nc2c(=O)n1-c1cccc2cccc(Cl)c12 ; "PdCl2" ; Cc1nc2c(N3CCNCC3)nc(OC[C@@H]3CCCN3C)nc2c(=O)n1-c1cccc2cccc(Cl)c12]

To a mixture of benzyl (S)-4-(7-(8-chloronaphthalen-1-yl)-6-methyl-2-((1-methylpyrrolidin-2-yl)methoxy)-8-oxo-7,8-dihydropyrimido[5,4-d]pyrimidin-4-yl)piperazine-1-carboxylate (63 mg, 0.0965 mmol) in DCM (2 mL) was added Et₃SiH (45 mg, 0.386 mmol) and Et₃N (39 mg, 0.386 mmol), followed by PdCl₂ (2 mg, 0.00964 mmol). The mixture was stirred at room temperature for 0.5 h under N₂. LCMS showed starting material was not consumed. Et₃SiH (45 mg, 0.386 mmol) and Et₃N (39 mg, 0.386 mmol) and PdCl₂ (8 mg, 0.0386 mmol) were added to the mixture. The mixture was stirred at room temperature for 0.5 h under N₂. LCMS showed the reaction was completed. The reaction mixture was quenched with H₂O (15 mL) and extracted with DCM/MeOH (10/1, 3×15 mL). The combined organic layers were dried over anhydrous Na₂SO₄, filtered and concentrated under reduced pressure to obtain crude (S)-3-(8-chloronaphthalen-1-yl)-2-methyl-6-((1-methylpyrrolidin-2-yl)methoxy)-8-(piperazin-1-yl)pyrimido[5,4-d]pyrimidin-4(3H)-one (50 mg, 100%) as a yellow oil, which was used directly for the next step without further purification.

LCMS: Rt: 0.549 min; MS m/z (ESI): 520.2 [M+H]⁺.

### Step 4: Synthesis of Compound 3

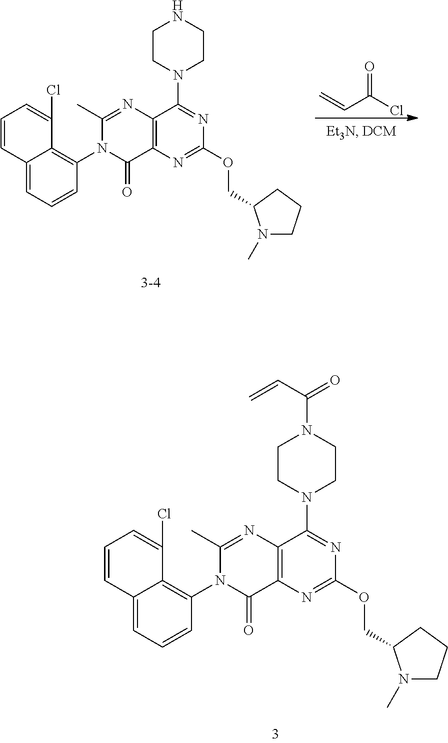

To a mixture of (S)-3-(8-chloronaphthalen-1-yl)-2-methyl-6-((1-methylpyrrolidin-2-yl)methoxy)-8-(piperazin-1-yl)pyrimido[5,4-d]pyrimidin-4(3H)-one (50 mg, 0.0965 mmol, 1.0 eq.) and Et₃N (29 mg, 0.2895 mmol, 3.0 eq.) in DCM (2 mL) was added dropwise a solution of acryloyl chloride (8.8 mg, 0.0965 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain (S)-8-(4-acryloylpiperazin-1-yl)-3-(8-chloronaphthalen-1-yl)-2-methyl-6-((1-methylpyrrolidin-2-yl)methoxy)pyrimido[5,4-d]pyrimidin-4(3H)-one (6 mg, 10.9%, 3).

LCMS: Rt: 0.825 min; MS m/z (ESI): 574.3 [M+H]⁺;

¹H NMR (400 MHz, DMSO) δ 8.27 (s, 0.89H), 8.26-8.23 (m, 1H), 8.18-8.10 (m, 1H), 7.82-7.55 (m, 4H), 6.85 (dd, J=16.6, 10.4 Hz, 1H), 6.17 (dd, J=16.7, 2.1 Hz, 1H), 5.73 (dd, J=10.4, 2.2 Hz, 1H), 4.47-4.17 (m, 5H), 4.15-4.09 (m, 1H), 3.83-3.70 (m, 4H), 2.98-2.93 (m, 1H), 2.59-2.53 (m, 1H), 2.35 (s, 3H), 2.22-2.13 (m, 1H), 2.09-1.99 (m, 3H), 1.97-1.86 (m, 1H), 1.73-1.54 (m, 3H).

### Example 4

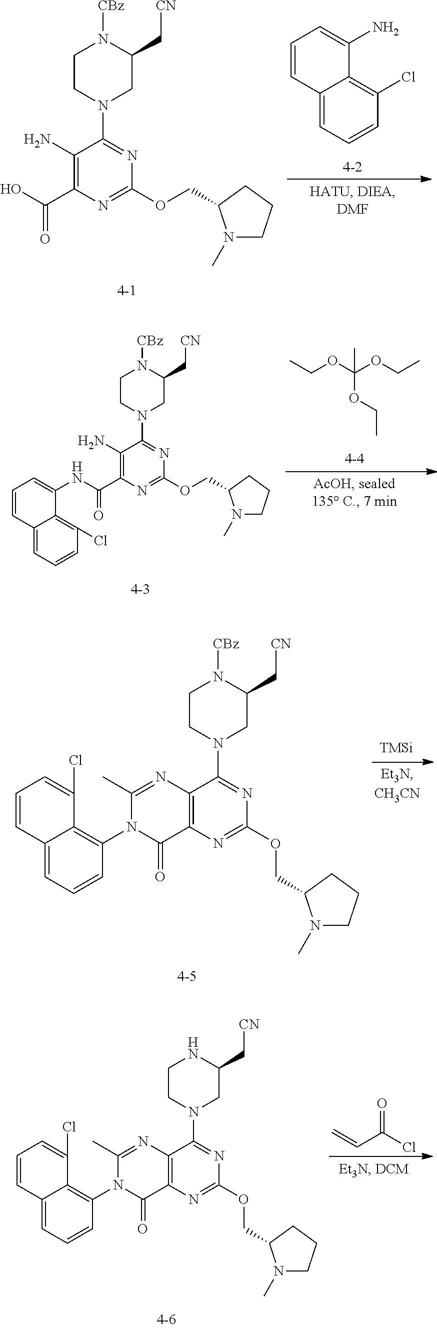

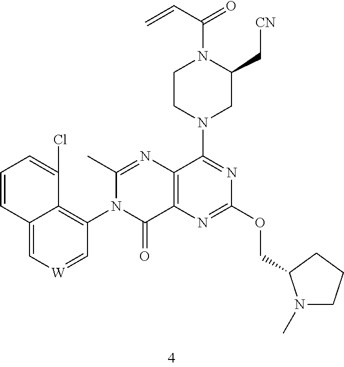

### Step 1: Synthesis of Compound 4-3

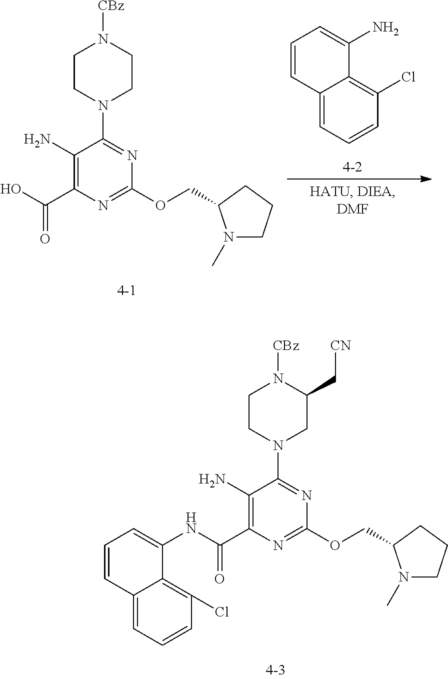

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (200 mg, 0.393 mmol, 1.0 eq.) and 8-chloronaphthalen-1-amine (49 mg, 0.275 mmol, 1.0 eq.) in anhydrous DMF (5.0 mL) was added DIEA (152 mg, 1.179 mmol, 3.0 eq.), followed by the addition of HATU (149 mg, 0.393 mmol, 1.0 eq.). The reaction mixture was stirred at 60° C. under Ar for 1 h. LCMS showed starting material was consumed and desired product formed. The reaction mixture was cooled to room temperature, diluted with EtOAc (30 mL) and washed with brine (3×30 mL), dried over anhydrous Na₂SO₄, filtered and concentrated under reduced pressure. The residue was purified by silica column chromatography eluting with DCM/MeOH (1/0-10:1, v/v) to obtain benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (200 mg, 51%).

LCMS: Rt: 0.955 min; MS m/z (ESI): 669.3 [M+H]⁺.

### Step 2: Synthesis of Compound 4-5

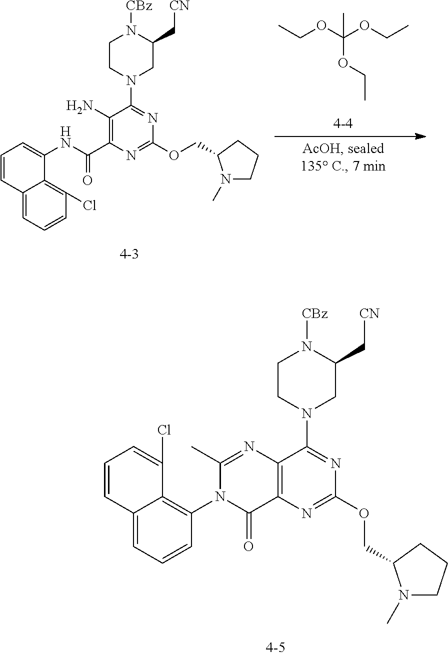

To a mixture of (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (90 mg, 0.1348 mmol, 1.0 eq.) and AcOH (0.8 mL) was added 1,1,1-triethoxyethane (332 mg, 2.020 mmol, 15.0 eq.). The mixture was stirred at 135° C. in a sealed tube for 8 min. LCMS showed starting material was consumed and desired product formed. The reaction mixture was adjusted to pH=8-9 with aq. NaHCO₃ solution and extracted with DCM (3×20 mL). The combined organic layers were dried over anhydrous Na₂SO₄, filtered and concentrated under reduced pressure. The residue was purified by prep-TLC eluting with DCM/MeOH (10:1, v/v) to obtainbenzyl (S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)-2-(cyanomethyl)piperazine-1-carboxylate (64 mg, 62%).

LCMS: Rt: 0.929 min; MS m/z (ESI): 693.0 [M+H]⁺.

### Step 3: Synthesis of Compound 4-6

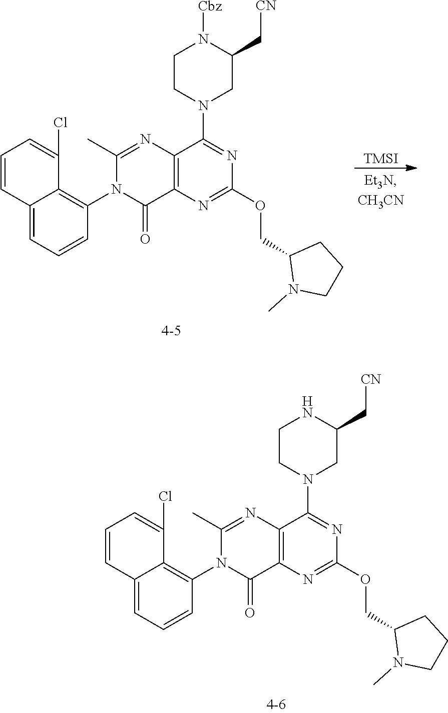 [SMILES: "TMSI" ; Cc1nc2c(N3CCN(C(=O)OCc4ccccc4)[C@@H](CC#N)C3)nc(OC[C@@H]3CCCN3C)nc2c(=O)n1-c1cccc2cccc(Cl)c12 ; Cc1nc2c(N3CCN[C@@H](CC#N)C3)nc(OC[C@@H]3CCCN3C)nc2c(=O)n1-c1cccc2cccc(Cl)c12]

To a mixture of benzyl (S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)-2-(cyanomethyl)piperazine-1-carboxylate (64 mg, 0.0925 mmol, 1 eq.) in CH₃CN (5.0 mL) was added TMSI (148 mg, 0.740 mmol, 8 eq.), and the mixture was stirred at 35° C. for 1 h under N₂. LCMS showed the starting material was consumed. The resulting mixture was added with Et₃N (149 mg, 1.48 mmol, 16 eq.) and stirred at room temperature for 15 min. The mixture was concentrated under reduced pressure. The residue was diluted with H₂O (15 mL) and extracted with DCM/MeOH (10/1, 3×15 mL). The combined organic layers were dried over anhydrous Na₂SO₄, filtered and concentrated under reduced pressure. The residue was purified by prep-TLC eluting with DCM/MeOH (8:1, v/v) to obtain 2-((S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (50 mg, 96%).

LCMS: Rt: 0.379 min; MS m/z (ESI): 559.3 [M+H]⁺.

### Step 4: Synthesis of Compound 4

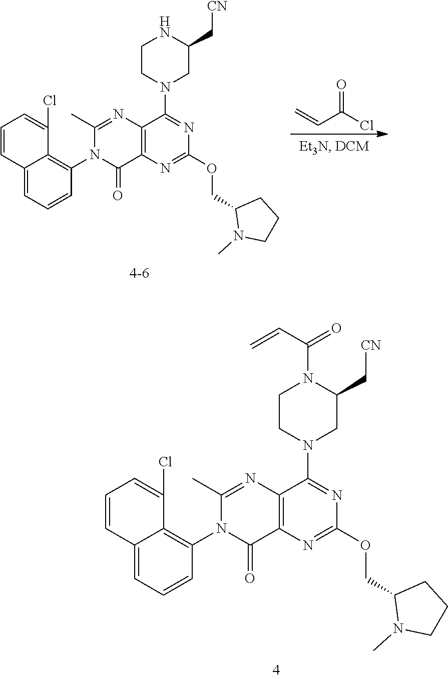

To a mixture of 2-((S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (50 mg, 0.085 mmol, 1.0 eq.) and Et₃N (26 mg, 0.255 mmol, 3.0 eq.) in DCM (2 mL) was added dropwise a solution of acryloyl chloride (7.7 mg, 0.085 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain 2-((S)-1-acryloyl-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (20 mg, 38%, 4).

LCMS: Rt: 0.996 min; MS m/z (ESI): 580.2 [M+H]⁺;

¹H NMR (400 MHz, DMSO) δ 8.27 (d, J=8.0 Hz, 1H), 8.20 (s, 0.64H), 8.16 (d, J=8.1 Hz, 1H), 7.86-7.67 (m, 3H), 7.60 (t, J=7.9 Hz, 1H), 6.95-6.80 (m, 1H), 6.21 (d, J=16.5 Hz, 1H), 5.79 (d, J=10.2 Hz, 1H), 5.65-4.70 (m, 3H), 4.53-4.31 (m, 1.5H), 4.22-4.12 (m, 1.5H), 3.76-3.41 (m, 2H), 3.25-2.90 (m, 4H), 2.75-2.65 (m, 1H), 2.41 (s, 3H), 2.34-2.23 (m, 1H), 2.10 (d, J=1.3 Hz, 3H), 2.00-1.92 (m, 1H), 1.77-1.57 (m, 3H).

### Example 5

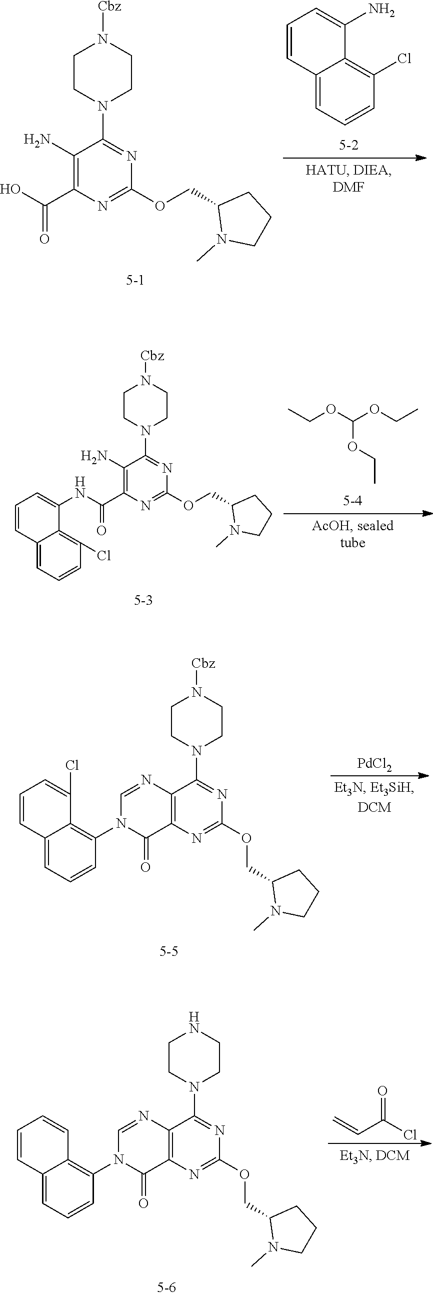

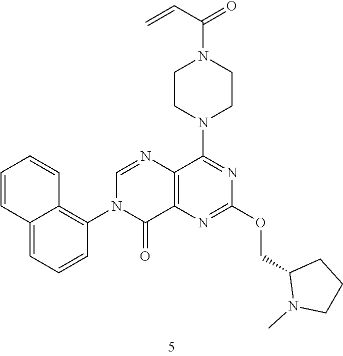 [SMILES: C=CC(=O)N1CCN(c2nc(OC[C@@H]3CCCN3C)nc3c(=O)n(-c4cccc5ccccc45)cnc23)CC1]

### Step 1: Synthesis of Compound 5-3

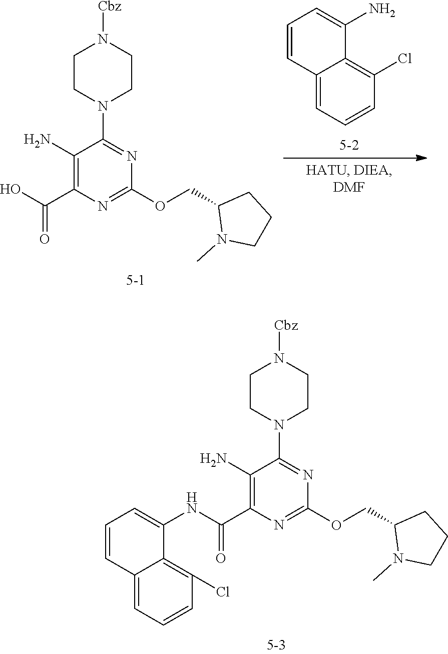 [SMILES: CN1CCC[C@H]1COc1nc(C(=O)O)c(N)c(N2CCN(C(=O)OCc3ccccc3)CC2)n1 ; Nc1cccc2cccc(Cl)c12 ; CN1CCC[C@H]1COc1nc(C(=O)Nc2cccc3cccc(Cl)c23)c(N)c(N2CCN(C(=O)OCc3ccccc3)CC2)n1]

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (120 mg, 0.255 mmol, 1.0 eq.) and 8-chloronaphthalen-1-amine (68 mg, 0.383 mmol, 1.5 eq.) in anhydrous DMF (3.0 mL) was added DIEA (99 mg, 0.765 mmol, 3.0 eq.), followed by the addition of HATU (194 mg, 0.51 mmol, 2.0 eq.). The reaction mixture was stirred at 60° C. under Ar for 1 h. LCMS showed the reaction was completed. The reaction mixture was cooled to room temperature, diluted with EtOAc (30 mL) and washed with brine (3×30 mL), dried over anhydrous Na₂SO₄, filtered and concentrated under reduced pressure. The residue was purified by preparative TLC with DCM/MeOH (10:1, v/v) to obtain benzyl (S)-4-(5-amino-6-((8-chloronaphthalen-1-yl)carbamoyl)-2-((1-methylpyrrolidin-2-yl)methoxy)pyrimidin-4-yl)piperazine-1-carboxylate (80 mg, 50%).

LCMS: Rt: 0.951 min; MS m/z (ESI): 630.2 [M+H]⁺.

### Step 2: Synthesis of Compound 5-5

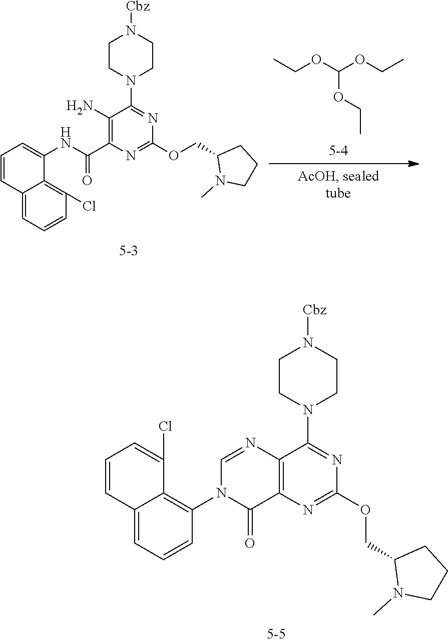

To a mixture of benzyl (S)-4-(5-amino-6-((8-chloronaphthalen-1-yl)carbamoyl)-2-((1-methylpyrrolidin-2-yl)methoxy)pyrimidin-4-yl)piperazine-1-carboxylate (90 mg, 0.143 mmol, 1.0 eq.) in AcOH (0.8 mL) was added triethoxymethane (317 mg, 2.145 mmol, 15.0 eq.). The mixture was stirred at 135° C. in a sealed tube for 8 min. LCMS showed starting material was consumed and desired product formed. The reaction mixture was adjusted to pH=8-9 with aq. NaHCO₃ solution and extracted with DCM (3×15 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-4-(7-(8-chloronaphthalen-1-yl)-2-((1-methylpyrrolidin-2-yl)methoxy)-8-oxo-7,8-dihydropyrimido[5,4-d]pyrimidin-4-yl)piperazine-1-carboxylate (50 mg, 55%).

LCMS: Rt: 0.951 min; MS m/z (ESI): 640.2 [M+H]⁺.

### Step 3: Synthesis of Compound 5-6

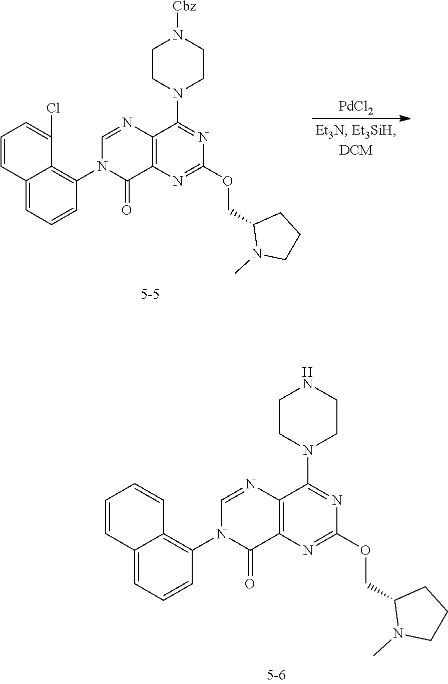 [SMILES: CN1CCC[C@H]1COc1nc(N2CCN(C(=O)OCc3ccccc3)CC2)c2ncn(-c3cccc4cccc(Cl)c34)c(=O)c2n1 ; "PdCl2" ; CN1CCC[C@H]1COc1nc(N2CCNCC2)c2ncn(-c3cccc4ccccc34)c(=O)c2n1]

To a mixture of benzyl (S)-4-(7-(8-chloronaphthalen-1-yl)-2-((1-methylpyrrolidin-2-yl)methoxy)-8-oxo-7,8-dihydropyrimido[5,4-d]pyrimidin-4-yl)piperazine-1-carboxylate (50 mg, 0.078 mmol) in DCM (3 mL) was added Et₃SiH (73 mg, 0.626 mmol) and Et₃N (63 mg, 0.626 mmol), followed by PdCl₂ (4.1 mg, 0.0235 mmol). The mixture was stirred at room temperature for 1 h under N₂. LCMS showed that 30% desired MS was observed. Et₃SiH (73 mg, 0.626 mmol) and Et₃N (63 mg, 0.626 mmol) and PdCl₂ (8 mg, 0.047 mmol). were added to the mixture. The mixture was stirred at room temperature for 1 h under N₂. LCMS showed de-Cl product was observed. The reaction mixture was quenched with H₂O (15 mL) and extracted with DCM/MeOH (10/1, 3×15 mL). The combined organic layers were dried over anhydrous Na₂SO₄, filtered and concentrated under reduced pressure to obtain crude (S)-6-((1-methylpyrrolidin-2-yl)methoxy)-3-(naphthalen-1-yl)-8-(piperazin-1-yl)pyrimido[5,4-d]pyrimidin-4(3H)-one (36.7 mg, 100%) as a yellow oil, which was used directly for the next step without further purification.

LCMS: Rt: 0.385 min; MS m/z (ESI): 472.2 [M+H]⁺.

### Step 4: Synthesis of Compound 5

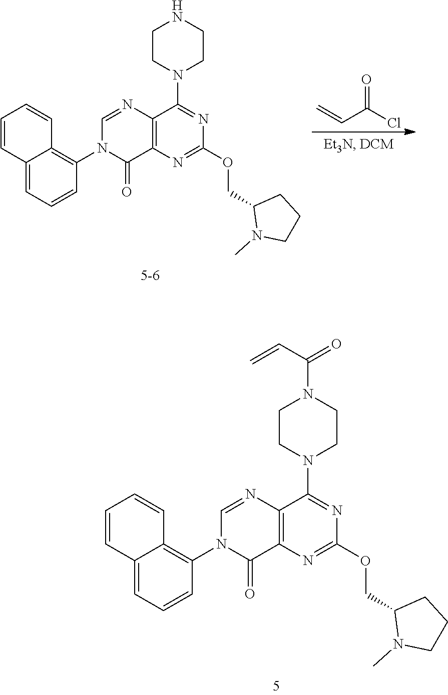

To a mixture of (S)-6-((1-methylpyrrolidin-2-yl)methoxy)-3-(naphthalen-1-yl)-8-(piperazin-1-yl)pyrimido[5,4-d]pyrimidin-4(3H)-one (36.7 mg, 0.078 mmol, 1.0 eq.) and Et₃N (24 mg, 0.234 mmol, 3.0 eq.) in DCM (2 mL) was added dropwise a solution of acryloyl chloride (5.7 mg, 0.0624 mmol, 0.8 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain (S)-8-(4-acryloylpiperazin-1-yl)-6-((1-methylpyrrolidin-2-yl)methoxy)-3-(naphthalen-1-yl)pyrimido[5,4-d]pyrimidin-4(3H)-one (7 mg, 17%, 5).

LCMS: Rt: 0.819 min; MS m/z (ESI): 526.2 [M+H]⁺;

¹H NMR (400 MHz, DMSO) δ 8.29 (s, 0.93H), 8.27 (s, 1H), 8.19-8.06 (m, 2H), 7.74-7.54 (m, 5H), 6.87 (dd, J=16.7, 10.4 Hz, 1H), 6.18 (dd, J=16.7, 2.3 Hz, 1H), 5.74 (dd, J=10.4, 2.3 Hz, 1H), 4.38-4.25 (m, 4H), 4.19-4.13 (m, 1H), 3.84-3.69 (m, 5H), 2.98-2.92 (m, 1H), 2.64-2.53 (m, 1H), 2.36 (s, 3H), 2.19 (q, J=8.6 Hz, 1H), 2.00-1.91 (m, 1H), 1.74-1.59 (m, 3H).

### Example 6

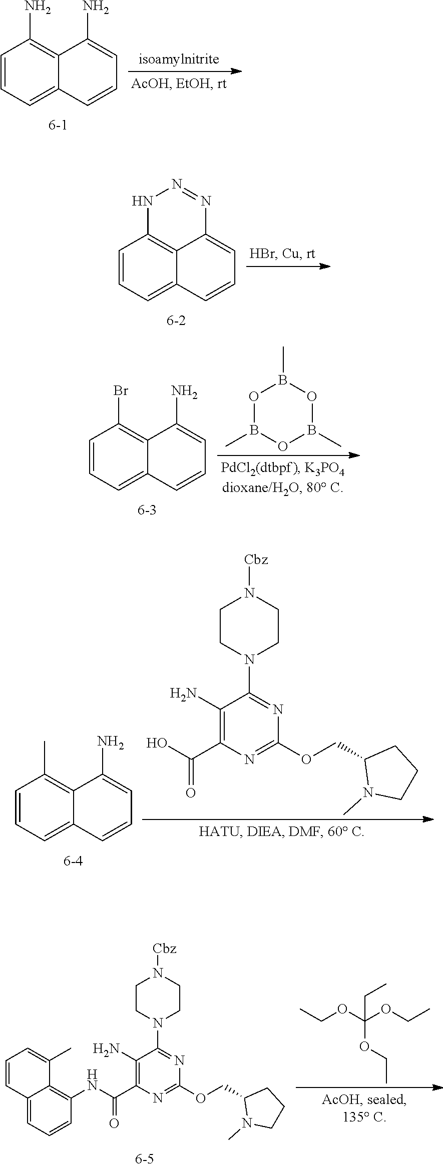

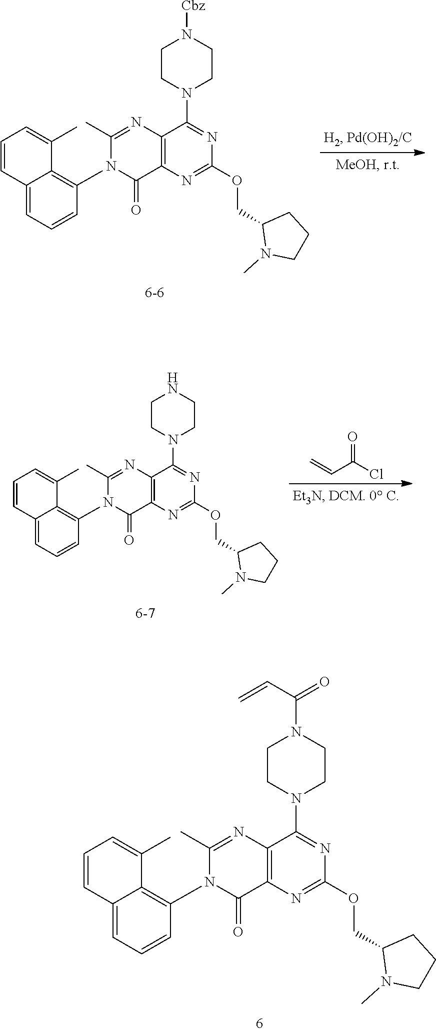

### Step 1: Synthesis of Compound 6-2

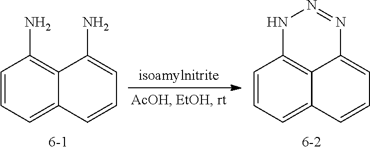 [SMILES: Nc1cccc2cccc(N)c12 ; c1cc2c3c(cccc3c1)NN=N2]

To a cooled (0° C.) solution of naphthalene-1,8-diamine (20 g, 126.58 mmol, 1.0 eq.) in EtOH (400 mL) and AcOH (40 mL) was added dropwise isoamylnitrite (16.6 mL, 124.05 mmol, 0.98 eq.). After addition, the reaction mixture was stirred at room temperature for overnight. LCMS analysis showed starting material was consumed and desired product formed. The solid was collected by filtration, washed with EtOH (200 mL) and dried under vacuum to obtain 1H-naphtho[1,8-de][1,2,3]triazine (18 g, 86%), which was used directly for the next step.

LCMS (ESI, m/z): [M+1]⁺=170; RT=1.219 min.

### Step 2: Synthesis of Compound 6-3

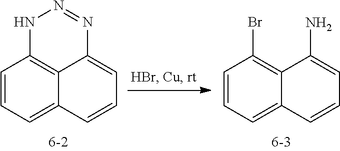 [SMILES: c1cc2c3c(cccc3c1)NN=N2 ; Nc1cccc2cccc(Br)c12]

To a cooled (0° C.) mixture of copper turnings (0.5 g, 7.81 mmol, 0.07 eq.) in aq.HBr (48%, 200 mL) was added slowly 1H-naphtho[1,8-de][1,2,3]triazine (18 g, 106.51 mmol, 1.0 eq.). After addition, the reaction mixture was stirred at room temperature for overnight. LCMS analysis showed starting material was consumed and desired product formed. The reaction mixture was diluted with EtOAc (50 mL), followed by the addition of aq. KOH (45%, w/w) to adjust pH=11-12. The organic layer of the filtrate was separated and the aqueous layer was extracted with EtOAc (50 mL×2). The combined organics were dried over anhydrous Na₂SO₄ and concentrated to obtain 8-bromonaphthalen-1-amine (15.8 g, 67%), which was used directly for the next step.

LCMS (ESI, m/z): [M+1]⁺=222; RT=1.575 min.

### Step 3: Synthesis of Compound 6-4

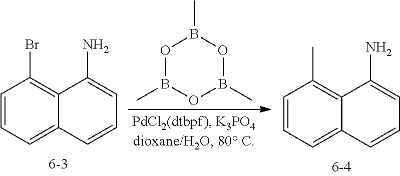

To a solution of 8-bromonaphthalen-1-amine (6 g, 27.15 mmol, 1.0 eq.) and 2,4,6-trimethyl-1,3,5,2,4,6-trioxatriborinane (10.22 g, 81.45 mmol, 3.0 eq.) in dioxane (40 mL) and H₂O (10 mL) was added PdCl₂(dtbpf) (0.89 g, 1.36 mmol, 0.05 eq.) and K₃PO₄ (17.27 g, 81.45 mmol, 3.0 eq.). The mixture was stirred at 80° C. under Ar for overnight. LCMS analysis showed starting material was consumed and desired product formed. The reaction mixture was filtered through celite and the filtrate was concentrated to dryness. The residue was purified by silica column chromatography eluting with EtOAc/Pet.ether (10%, v/v) to obtain 8-methylnaphthalen-1-amine (1.2 g, 29%).

LCMS (ESI, m/z): [M+1]⁺=158; RT=1.253 min.

### Step 4: Synthesis of Compound 6-5

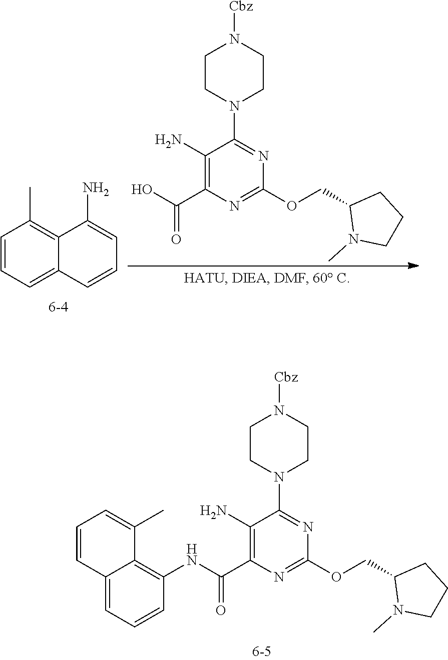 [SMILES: Cc1cccc2cccc(N)c12 ; CN1CCC[C@H]1COc1nc(C(=O)O)c(N)c(N2CCN(C(=O)OCc3ccccc3)CC2)n1 ; Cc1cccc2cccc(NC(=O)c3nc(OC[C@@H]4CCCN4C)nc(N4CCN(C(=O)OCc5ccccc5)CC4)c3N)c12]

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (150 mg, 0.319 mmol, 1.0 eq.) and 8-methylnaphthalen-1-amine HCl (62 mg, 0.319 mmol, 1.0 eq.) in anhydrous DMF (5.0 mL) was added DIEA (123 mL, 0.957 mmol, 3.0 eq.), followed by the addition of HATU (121 mg, 0.319 mmol, 1.0 eq.). The reaction mixture was stirred at 60° C. under Ar for 1 h. LCMS showed starting material was consumed and desired product formed. The reaction mixture was cooled to room temperature, diluted with EtOAc (30 mL) and washed with brine (3×30 mL), dried over anhydrous Na₂SO₄, filtered and concentrated under reduced pressure. The residue was purified by preparative TLC with DCM/MeOH (1/0-10:1, v/v) to obtain benzyl (S)-4-(5-amino-6-((8-methylnaphthalen-1-yl)carbamoyl)-2-((1-methylpyrrolidin-2-yl)methoxy)pyrimidin-4-yl)piperazine-1-carboxylate (56 mg, 29%).

LCMS (ESI, m/z): [M+1]⁺=610; RT=0.943 min.

### Step 5: Synthesis of Compound 6-6

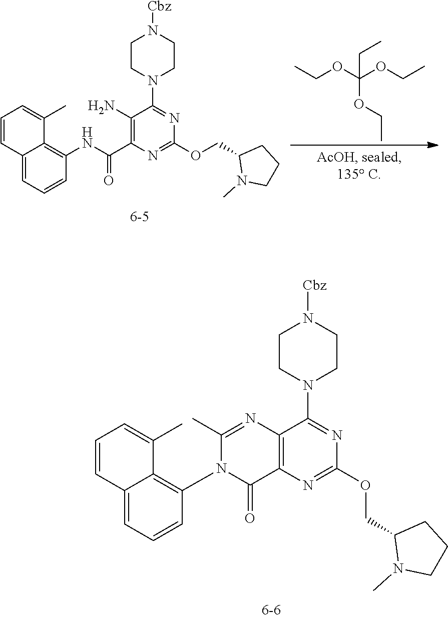 [SMILES: Cc1cccc2cccc(NC(=O)c3nc(OC[C@@H]4CCCN4C)nc(N4CCN(C(=O)OCc5ccccc5)CC4)c3N)c12 ; CCOC(CC)(OCC)OCC ; Cc1cccc2cccc(-n3c(C)nc4c(N5CCN(C(=O)OCc6ccccc6)CC5)nc(OC[C@@H]5CCCN5C)nc4c3=O)c12]

To a solution of benzyl (S)-4-(5-amino-6-((8-methylnaphthalen-1-yl)carbamoyl)-2-((1-methylpyrrolidin-2-yl)methoxy)pyrimidin-4-yl)piperazine-1-carboxylate (20 mg, 0.033 mmol, 1.0 eq.) and AcOH (0.2 mL) was added 1,1,1-triethoxyethane (78 mg, 0.493 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 20 mL) to adjust pH=7-8, which was extracted with DCM (10 mL×3). The combined organic fractions were dried over anhydrous Na₂SO₄ and concentrated. The residue was purified by prep-TLC eluting with DCM/MeOH (10:1, v/v) to obtain benzyl (S)-4-(6-methyl-7-(8-methylnaphthalen-1-yl)-2-((1-methylpyrrolidin-2-yl)methoxy)-8-oxo-7,8-dihydropyrimido[5,4-d]pyrimidin-4-yl)piperazine-1-carboxylate (13 mg, 64%).

LCMS (ESI, m/z): [M+1]⁺=634; RT=1.230 min.

### Step 6: Synthesis of Compound 6-7

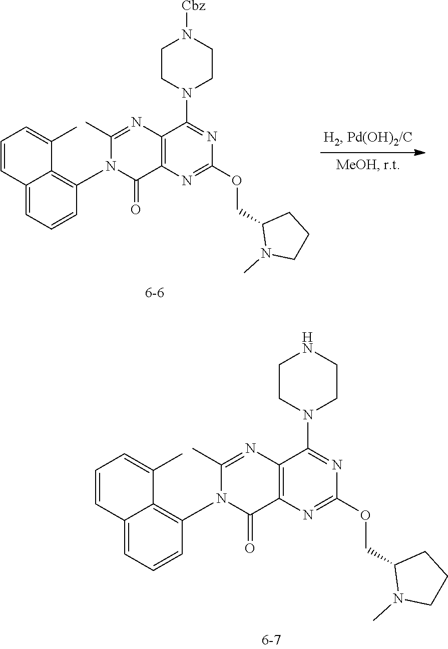 [SMILES: Cc1cccc2cccc(-n3c(C)nc4c(N5CCN(C(=O)OCc6ccccc6)CC5)nc(OC[C@@H]5CCCN5C)nc4c3=O)c12 ; Cc1cccc2cccc(-n3c(C)nc4c(N5CCNCC5)nc(OC[C@@H]5CCCN5C)nc4c3=O)c12]

To a solution of benzyl (S)-4-(6-methyl-7-(8-methylnaphthalen-1-yl)-2-((1-methylpyrrolidin-2-yl)methoxy)-8-oxo-7,8-dihydropyrimido[5,4-d]pyrimidin-4-yl)piperazine-1-carboxylate (43 mg, 0.118 mmol, 1.0 eq.) in MeOH (0.2 mL) was added Pd(OH)₂/C (20% on carbon, wetted with ca.50% water, 8.4 mg, 0.012 mmol, 0.1 eq.), and the mixture was stirred at room temperature for 1 h under H₂. LCMS showed starting material was consumed and desired product formed. The reaction mixture was filtered and the filtrate was concentrated to dryness to obtain (S)-2-methyl-3-(8-methylnaphthalen-1-yl)-6-((1-methylpyrrolidin-2-yl)methoxy)-8-(piperazin-1-yl)pyrimido[5,4-d]pyrimidin-4(3H)-one (33 mg, 99%), which was used directly for the next step.

LCMS (ESI, m/z): [M+1]⁺=500; RT=0.664 min;

### Step 7: Synthesis of Compound 6

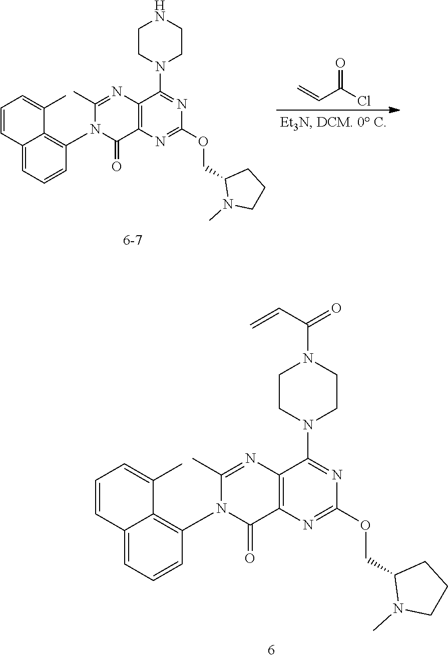

To a cooled (0° C.) solution of (S)-2-methyl-3-(8-methylnaphthalen-1-yl)-6-((1-methylpyrrolidin-2-yl)methoxy)-8-(piperazin-1-yl)pyrimido[5,4-d]pyrimidin-4(3H)-one (33 mg, 0.067 mmol, 1.0 eq.) and Et₃N (20.2 mg, 0.200 mmol, 3.0 eq.) in DCM (2 mL) was added dropwise a solution of acryloyl chloride (60 mg, 0.067 mmol, 1 eq.) in DCM (5.0 mL). After addition, the mixture was stirred at 0° C. for 30 min. LCMS showed starting material was consumed and desired product formed. Water (20 mL) was added and the organic layer was separated. The aqueous layer was extracted with DCM (10 mL×3). The combined organic fractions were dried over anhydrous Na₂SO₄ and concentrated. The residue was purified by prep-HPLC (ACN-H₂O+0.1% HCOOH) to obtain (S)-8-(4-acryloylpiperazin-1-yl)-2-methyl-3-(8-methylnaphthalen-1-yl)-6-((1-methylpyrrolidin-2-yl)methoxy)pyrimido[5,4-d]pyrimidin-4(3H)-one (HCOOH salt, 5.6 mg, 15%, 6·HCOOH) (C₃₁H₃₅N₇O₃·HCOOH).

LCMS (ESI, m/z): [M+1]⁺=554; RT=1.014 min;

¹H NMR (400 MHz, DMSO-d₆) δ 8.25 (d, J=8.0 Hz, 1H), 8.14 (dd, J=8.4, 1.2 Hz, 1H), 7.98 (d, J=8.0 Hz, 1H), 7.71-7.62 (m, 1H), 7.59 (d, J=7.6 Hz, 1H), 7.54-7.45 (m, 1H), 7.39 (d, J=6.8 Hz, 1H), 6.85 (dd, J=16.8, 10.8 Hz, 1H), 6.17 (dd, J=16.8, 2.8 Hz, 1H), 5.73 (dd, J=10.4, 2.4 Hz, 1H), 4.32 (dd, J=10.4, 4.8 Hz, 4H), 4.17-4.10 (m, 1H), 3.85-3.64 (m, 5H), 2.96 (d, J=4.8 Hz, 1H), 2.56 (d, J=6.8 Hz, 1H), 2.36 (s, 3H), 2.19 (d, J=5.6 Hz, 4H), 2.02 (s, 3H), 1.97-1.90 (m, 1H), 1.73-1.57 (m, 3H).

### Example 7

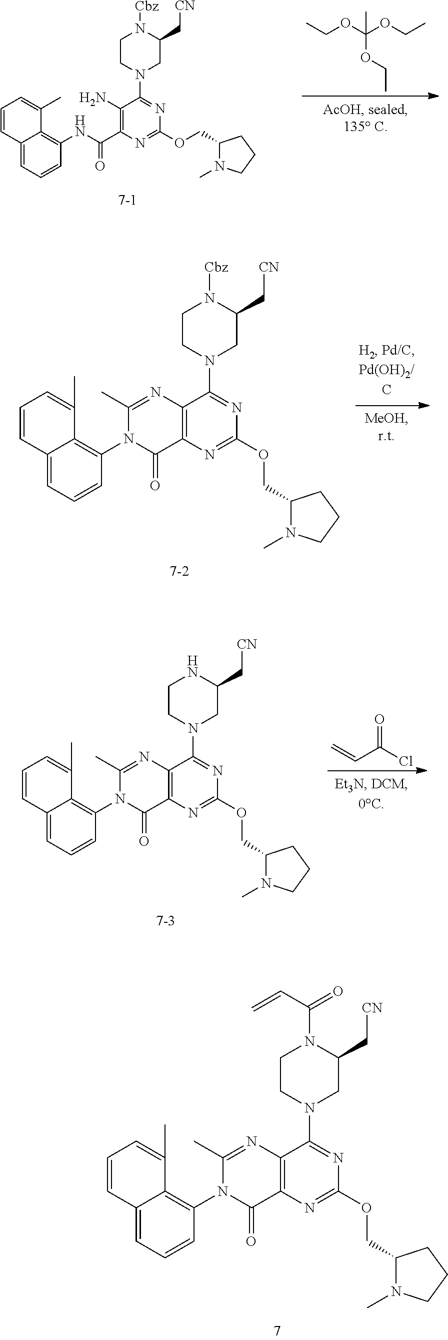

### Step 1: Synthesis of Compound 7-2

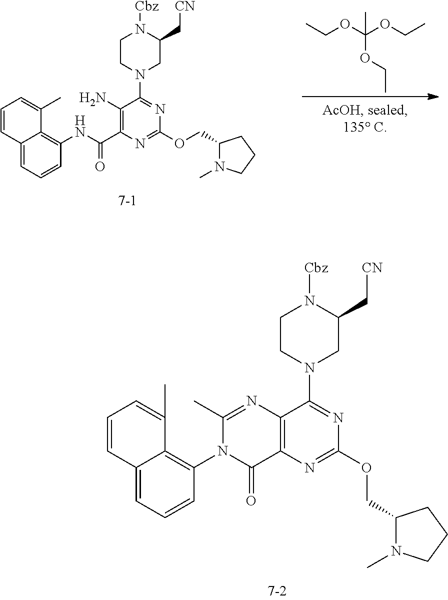 [SMILES: Cc1cccc2cccc(NC(=O)c3nc(OC[C@@H]4CCCN4C)nc(N4CCN(C(=O)OCc5ccccc5)[C@@H](CC#N)C4)c3N)c12 ; CCOC(C)(OCC)OCC ; Cc1cccc2cccc(-n3c(C)nc4c(N5CCN(C(=O)OCc6ccccc6)[C@@H](CC#N)C5)nc(OC[C@@H]5CCCN5C)nc4c3=O)c12]

To a mixture of benzyl (S)-4-(5-amino-6-((8-methylnaphthalen-1-yl)carbamoyl)-2-(((S)-1-methylpyrrolidin-2-yl)methoxy)pyrimidin-4-yl)-2-(cyanomethyl)piperazine-1-carboxylate (1) (230 mg, 0.354 mmol, 1.0 eq.) and AcOH (2.5 mL) was added 1,1,1-triethoxyethane (863 mg, 5.32 mmol, 15.0 eq.). The mixture was stirred at 135° C. in a sealed tube for 4.5 min. LCMS showed starting material was consumed and desired product formed. The reaction mixture was quenched with aq. NaHCO₃ (sat. 90 mL) to adjust pH=7-8, which was extracted with DCM (30 mL×2). The combined organic fractions were dried over anhydrous Na₂SO₄ and concentrated. The residue was purified by silica column chromatography eluting with DCM/MeOH (15:1, v/v) to obtain benzyl (S)-2-(cyanomethyl)-4-(6-methyl-7-(8-methylnaphthalen-1-yl)-2-(((S)-1-methylpyrrolidin-2-yl)methoxy)-8-oxo-7,8-dihydropyrimido[5,4-d]pyrimidin-4-yl)piperazine-1-carboxylate (52 mg, 22%).

LCMS (ESI, m/z): [M+1]⁺=673; RT=1.233 min.

### Step 2: Synthesis of Compound 7-3

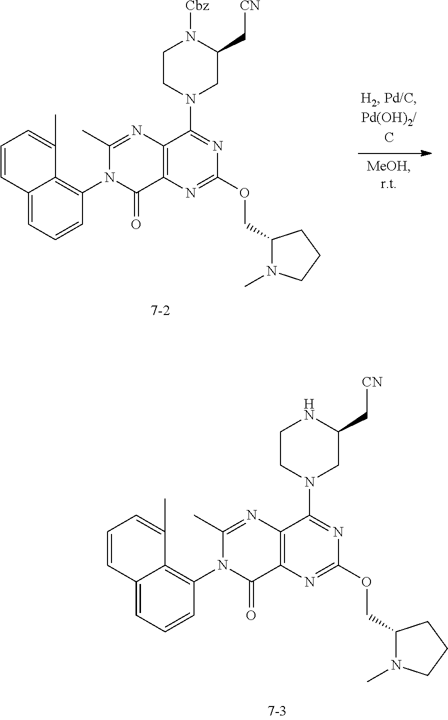 [SMILES: Cc1cccc2cccc(-n3c(C)nc4c(N5CCN(C(=O)OCc6ccccc6)[C@@H](CC#N)C5)nc(OC[C@@H]5CCCN5C)nc4c3=O)c12 ; Cc1cccc2cccc(-n3c(C)nc4c(N5CCN[C@@H](CC#N)C5)nc(OC[C@@H]5CCCN5C)nc4c3=O)c12]

To a solution of benzyl (S)-2-(cyanomethyl)-4-(6-methyl-7-(8-methylnaphthalen-1-yl)-2-(((S)-1-methylpyrrolidin-2-yl)methoxy)-8-oxo-7,8-dihydropyrimido[5,4-d]pyrimidin-4-yl)piperazine-1-carboxylate (2) (52 mg, 0.077 mmol, 1.0 eq.) in MeOH (5 mL) was added Pd/C (10% w/w, 8.2 mg, 0.0077 mmol, 0.1 eq.) and Pd(OH)₂/C (10% w/w, 11 mg, 0.0077 mmol, 0.1 eq.). The reaction mixture was stirred at room temperature under H₂ (balloon) for 1 h. LCMS showed starting material was consumed and desired product formed. The mixture was filtered through celite and the filtrate was concentrated to dryness to obtain 2-((S)-4-(6-methyl-7-(8-methylnaphthalen-1-yl)-2-(((S)-1-methylpyrrolidin-2-yl)methoxy)-8-oxo-7,8-dihydropyrimido[5,4-d]pyrimidin-4-yl)piperazin-2-yl)acetonitrile (39 mg, 94%), which was used directly for the next step.

LCMS (ESI, m/z): [M+1]⁺=539; RT=0.809 min.

### Step 3: Synthesis of Compound 7

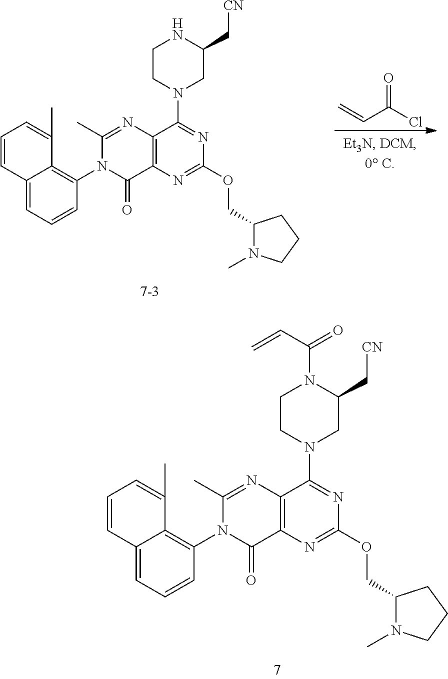

To a cooled (0° C.) solution of 2-((S)-4-(6-methyl-7-(8-methylnaphthalen-1-yl)-2-(((S′)-1-methylpyrrolidin-2-yl)methoxy)-8-oxo-7,8-dihydropyrimido[5,4-d]pyrimidin-4-yl)piperazin-2-yl)acetonitrile (3) (39 mg, 0.072 mmol, 1.0 eq.) and Et₃N (36 mg, 0.360 mmol, 5.0 eq.) in DCM (3.0 mL) was added dropwise a solution of acryloyl chloride (8 mg, 0.086 mmol, 1.2 eq.) in DCM (1.5 mL). After addition, the mixture was stirred at 0° C. for 30 min. LCMS showed most of starting material was consumed and desired product formed. Water (10 mL) was added and the organic layer was separated. The aqueous layer was extracted with DCM (5 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(6-methyl-7-(8-methylnaphthalen-1-yl)-2-(((S)-1-methylpyrrolidin-2-yl)methoxy)-8-oxo-7,8-dihydropyrimido[5,4-d]pyrimidin-4-yl)piperazin-2-yl)acetonitrile (HCOOH salt, 5.94 mg, 13%, 7·0.6HCOOH) (C₃₃H₃₆N₈O₃·0.6HCOOH).

LCMS (ESI, m/z): [M+1]⁺=593; RT=1.575 min.

¹H NMR (400 MHz, DMSO-d₆) δ 8.24 (s, 0.6H), 8.14 (d, J=8.0 Hz, 1H), 7.98 (d, J=8.0 Hz, 1H), 7.70-7.63 (m, 1H), 7.61-7.54 (m, 1H), 7.50 (t, J=7.6 Hz, 1H), 7.40 (d, J=7.2 Hz, 1H), 6.93-6.81 (m, 1H), 6.20 (dd, J=16.6, 2.2 Hz, 1H), 5.79 (d, J=11.2 Hz, 1H), 5.14-4.74 (m, 2H), 4.47 (s, 1H), 4.33 (dd, J=10.8, 4.8 Hz, 1H), 4.19-4.12 (m, 1H), 3.23-3.06 (m, 4H), 3.04-2.90 (m, 3H), 2.66-2.52 (m, 1H), 2.36 (s, 3H), 2.24-2.12 (m, 4H), 2.05 (s, 3H), 1.99-1.90 (m, 1H), 1.73-1.57 (m, 3H).

### Example 8

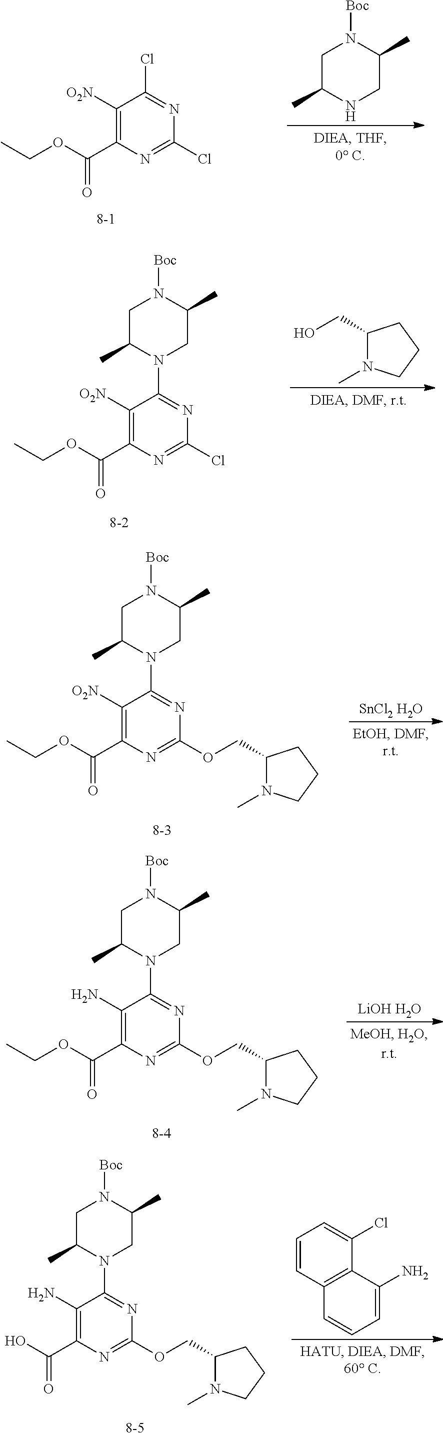

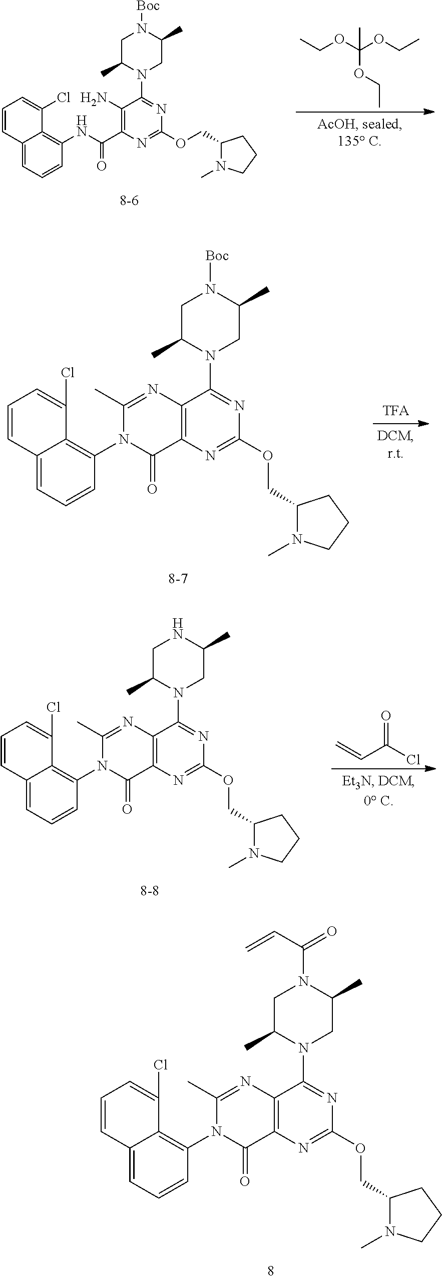

### Step 1: Synthesis of Compound 8-2

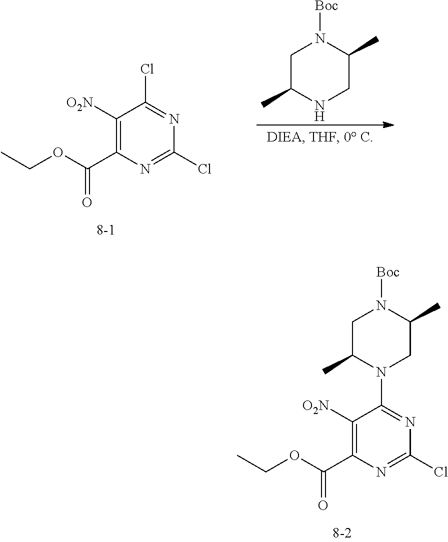

To a cooled (0° C.) solution of ethyl 2,6-dichloro-5-nitropyrimidine-4-carboxylate (1.00 g, 3.76 mmol, 1.0 eq.) (1) in anhydrous THF (15 mL) was added dropwise a solution of tert-butyl (2S,5S)-2,5-dimethylpiperazine-1-carboxylate (805 mg, 3.76 mmol, 1.0 eq.) and DIEA (0.93 mL, 5.64 mmol, 1.5 eq.) in anhydrous THF (15 mL). After addition, the reaction mixture was stirred at 0° C. for 40 min. TLC showed starting material was consumed and desired product was detected by LCMS. The reaction mixture was concentrated in vacuo. The residue was purified by silica column chromatography eluting with Pet.ether/EtOAc (10:1 to 4:1, v/v) to obtain ethyl 6-((2S,5S)-4-(tert-butoxycarbonyl)-2,5-dimethylpiperazin-1-yl)-2-chloro-5-nitropyrimidine-4-carboxylate (1.51 g, 90%).

LCMS (ESI, m/z): [M+1]⁺=444; RT=2.029 min.

### Step 2: Synthesis of Compound 8-3

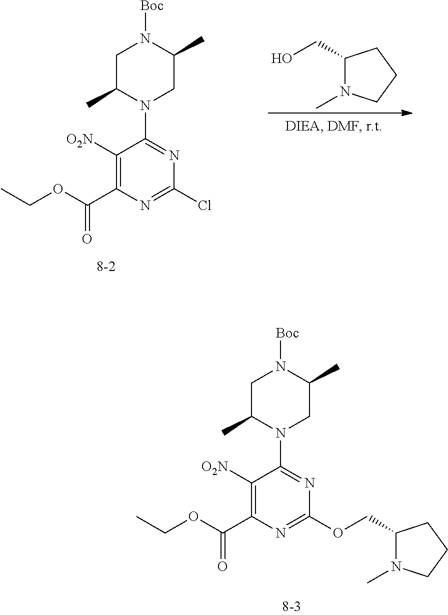

To a stirred mixture of ethyl 6-((2S,5S)-4-(tert-butoxycarbonyl)-2,5-dimethylpiperazin-1-yl)-2-chloro-5-nitropyrimidine-4-carboxylate (1.51 g, 3.40 mmol, 1.0 eq.) and DIEA (1.1 mL, 6.80 mmol, 2.0 eq.) in anhydrous DMF (10 mL) was added (S)-(1-methylpyrrolidin-2-yl)methanol (588 mg, 5.10 mmol, 1.5 eq.). The mixture was stirred at room temperature for 15 h. LCMS showed starting material was consumed and desired product formed. The reaction mixture was diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with DCM/MeOH (20:1, v/v) to obtain ethyl 6-((2S,5S)-4-(tert-butoxycarbonyl)-2,5-dimethylpiperazin-1-yl)-2-(((S)-1-methylpyrrolidin-2-yl)methoxy)-5-nitropyrimidine-4-carboxylate (1.51 g, 85%).

LCMS (ESI, m/z): [M+1]⁺=523; RT=1.168 min.

### Step 3: Synthesis of Compound 8-4

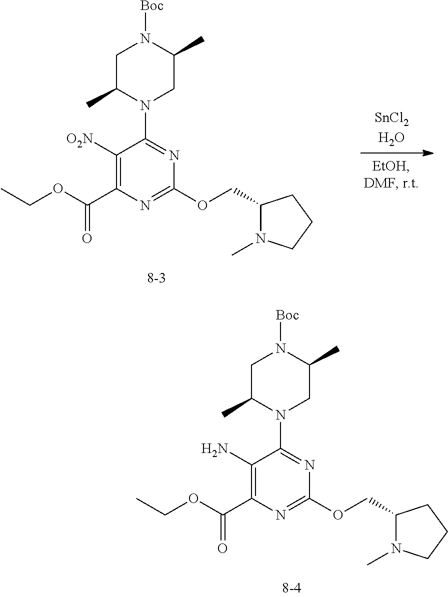 [SMILES: CCOC(=O)c1nc(OC[C@@H]2CCCN2C)nc(N2C[C@H](C)N(C(=O)OC(C)(C)C)C[C@@H]2C)c1[N+](=O)[O-] ; CCOC(=O)c1nc(OC[C@@H]2CCCN2C)nc(N2C[C@H](C)N(C(=O)OC(C)(C)C)C[C@@H]2C)c1N]

To a solution of ethyl 6-((2S,5S)-4-(tert-butoxycarbonyl)-2,5-dimethylpiperazin-1-yl)-2-(((S)-1-methylpyrrolidin-2-yl)methoxy)-5-nitropyrimidine-4-carboxylate (1.51 g, 2.89 mmol, 1.0 eq.) in anhydrous EtOH (48 mL)/DMF (16 mL) was added SnCl₂.2H₂O (3.26 g, 14.1 mmol, 5.0 eq.), and the mixture was stirred at room temperature under Ar for 15 h. LCMS showed most starting material was consumed and desired product formed. The reaction mixture was concentrated to remove EtOH and then diluted with EtOAc (80 mL), followed by the addition of aq. NaHCO₃ (sat. 120 mL). The resulting mixture was filtered through celite. The organic layer of the filtrate was separated and the aqueous layer was extracted with EtOAc (60 mL×2). The combined organic fractions were washed with brine (100 mL), dried over anhydrous Na₂SO₄ and concentrated. The residue was purified by silica column chromatography eluting with DCM/MeOH (15:1, v/v) to obtain ethyl 5-amino-6-((2S,5S)-4-(tert-butoxycarbonyl)-2,5-dimethylpiperazin-1-yl)-2-(((S)-1-methylpyrrolidin-2-yl)methoxy)pyrimidine-4-carboxylate (664 mg, 47%).

LCMS (ESI, m/z): [M+1]⁺=493; RT=1.090 min.

### Step 4: Synthesis of Compound 8-5

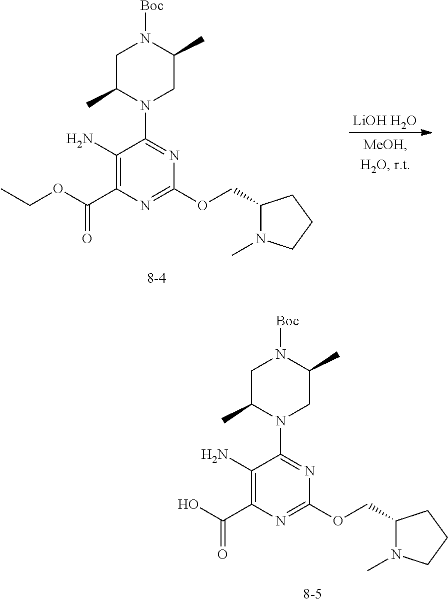 [SMILES: CCOC(=O)c1nc(OC[C@@H]2CCCN2C)nc(N2C[C@H](C)N(C(=O)OC(C)(C)C)C[C@@H]2C)c1N ; "LiOH H2O" ; C[C@H]1CN(c2nc(OC[C@@H]3CCCN3C)nc(C(=O)O)c2N)[C@@H](C)CN1C(=O)OC(C)(C)C]

To a solution of ethyl 5-amino-6-((2S,5S)-4-(tert-butoxycarbonyl)-2,5-dimethylpiperazin-1-yl)-2-(((S)-1-methylpyrrolidin-2-yl)methoxy)pyrimidine-4-carboxylate (664 mg, 1.35 mmol, 1.0 eq.) in MeOH (6.0 mL)/H₂O (1.0 mL) was added LiOH·H₂O (283 mg, 6.74 mmol, 5.0 eq.), and the mixture was stirred at room temperature for 2 h. LCMS showed starting material was consumed and desired product formed. The reaction mixture was acidified with aq. HCl (1 M) until pH=2-3, and then concentrated to dryness to obtain 5-amino-6-((2S,5S)-4-(tert-butoxycarbonyl)-2,5-dimethylpiperazin-1-yl)-2-(((S)-1-methylpyrrolidin-2-yl)methoxy) pyrimidine-4-carboxylic acid (918 mg), which was used directly for the next step.

LCMS (ESI, m/z): [M+1]⁺=465; RT=0.930 min.

### Step 5: Synthesis of Compound 8-6

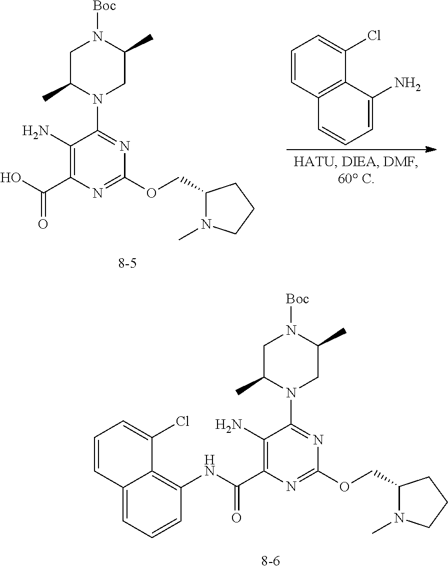 [SMILES: C[C@H]1CN(c2nc(OC[C@@H]3CCCN3C)nc(C(=O)O)c2N)[C@@H](C)CN1C(=O)OC(C)(C)C ; Nc1cccc2cccc(Cl)c12 ; C[C@H]1CN(c2nc(OC[C@@H]3CCCN3C)nc(C(=O)Nc3cccc4cccc(Cl)c34)c2N)[C@@H](C)CN1C(=O)OC(C)(C)C]

To a solution of 5-amino-6-((2S,5S)-4-(tert-butoxycarbonyl)-2,5-dimethylpiperazin-1-yl)-2-(((S)-1-methylpyrrolidin-2-yl)methoxy) pyrimidine-4-carboxylic acid (400 mg, 0.861 mmol, 1.0 eq.) and 8-chloronaphthalen-1-amine (92 mg, 0.517 mmol, 0.6 eq.) in anhydrous DMF (4.0 mL) was added DIEA (0.43 mL, 2.58 mmol, 3.0 eq.), followed by the addition of HATU (328 mg, 0.861 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄ and concentrated. The residue was purified by silica column chromatography eluting with DCM/MeOH (15:1, v/v) to obtain tert-butyl (2S,5S)-4-(5-amino-6-((8-chloronaphthalen-1-yl)carbamoyl)-2-(((S)-1-methylpyrrolidin-2-yl)methoxy)pyrimidin-4-yl)-2,5-dimethylpiperazine-1-carboxylate (110 mg, 20%).

LCMS (ESI, m/z): [M+1]⁺=624; RT=1.293 min.

### Step 6: Synthesis of Compound 8-7

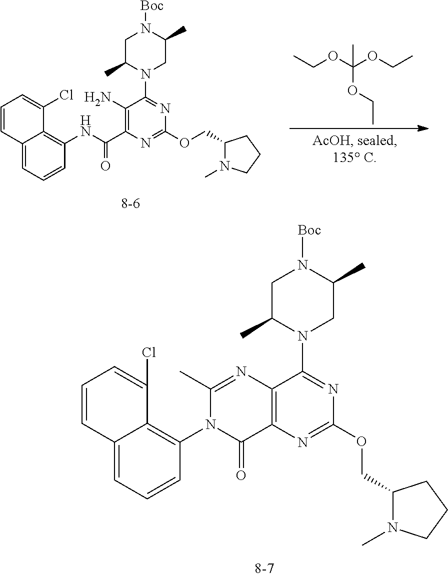 [SMILES: C[C@H]1CN(c2nc(OC[C@@H]3CCCN3C)nc(C(=O)Nc3cccc4cccc(Cl)c34)c2N)[C@@H](C)CN1C(=O)OC(C)(C)C ; CCOC(C)(OCC)OCC ; Cc1nc2c(N3C[C@H](C)N(C(=O)OC(C)(C)C)C[C@@H]3C)nc(OC[C@@H]3CCCN3C)nc2c(=O)n1-c1cccc2cccc(Cl)c12]

To a mixture of tert-butyl (2S,5S)-4-(5-amino-6-((8-chloronaphthalen-1-yl)carbamoyl)-2-(((S)-1-methylpyrrolidin-2-yl)methoxy)pyrimidin-4-yl)-2,5-dimethylpiperazine-1-carboxylate (110 mg, 0.176 mmol, 1.0 eq.) and AcOH (1.0 mL) was added 1,1,1-triethoxyethane (428 mg, 2.64 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 40 mL) to adjust pH=7-8, which was extracted with DCM (20 mL×3). The combined organic fractions were dried over anhydrous Na₂SO₄ and concentrated. The residue was purified by prep-TLC eluting with DCM/MeOH (10:1, v/v) to obtain tert-butyl (2S,5S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)-2,5-dimethylpiperazine-1-carboxylate (35 mg, 31%).

LCMS (ESI, m/z): [M+1]⁺=648; RT=1.286 min.

### Step 7: Synthesis of Compound 8-8

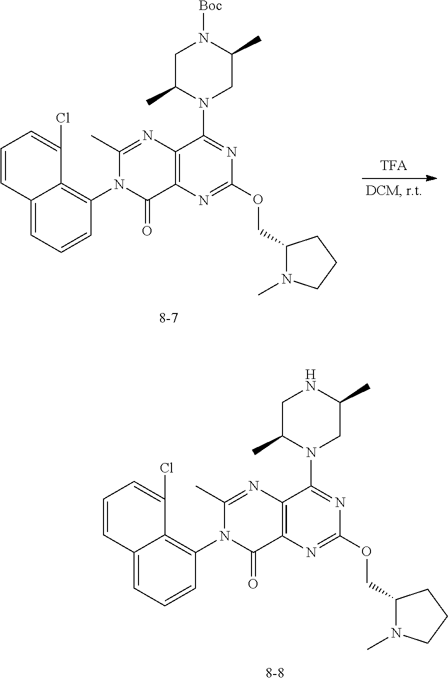 [SMILES: Cc1nc2c(N3C[C@H](C)N(C(=O)OC(C)(C)C)C[C@@H]3C)nc(OC[C@@H]3CCCN3C)nc2c(=O)n1-c1cccc2cccc(Cl)c12 ; Cc1nc2c(N3C[C@H](C)NC[C@@H]3C)nc(OC[C@@H]3CCCN3C)nc2c(=O)n1-c1cccc2cccc(Cl)c12]

To a solution of tert-butyl (2S,5S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)-2,5-dimethylpiperazine-1-carboxylate (35 mg, 0.054 mmol) in DCM (1.0 mL) was added TFA (1.0 mL), and the mixture was stirred at room temperature for 1 h. LCMS showed starting material was consumed and desired product formed. The reaction mixture was concentrated to dryness to obtain 3-(8-chloronaphthalen-1-yl)-8-((2S,5S)-2,5-dimethylpiperazin-1-yl)-2-methyl-6-(((S)-1-methylpyrrolidin-2-yl)methoxy)pyrimido[5,4-d] pyrimidin-4(3H)-one (TFA salt, 32 mg, 91%), which was used directly for the next step.

LCMS (ESI, m/z): [M+1]⁺=548; RT=0.709 min.

### Step 8: Synthesis of Compound 8

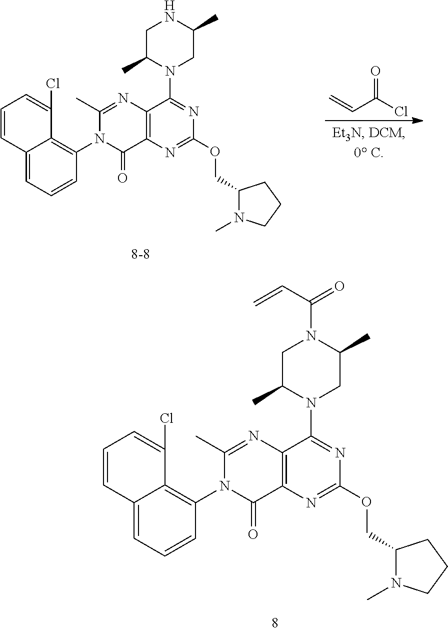

To a cooled (0° C.) solution of 3-(8-chloronaphthalen-1-yl)-8-((2S,5S)-2,5-dimethylpiperazin-1-yl)-2-methyl-6-(((S)-1-methylpyrrolidin-2-yl)methoxy)pyrimido[5,4-d] pyrimidin-4(3H)-one (TFA salt, 32 mg, 0.048 mmol, 1.0 eq.) and Et₃N (24 mg, 0.242 mmol, 5.0 eq.) in DCM (2.5 mL) was added dropwise a solution of acryloyl chloride (5.2 mg, 0.058 mmol, 1.2 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) to obtain 8-((2S,5S)-4-acryloyl-2,5-dimethylpiperazin-1-yl)-3-(8-chloronaphthalen-1-yl)-2-methyl-6-(((S)-1-methylpyrrolidin-2-yl)methoxy)pyrimido[5,4-d]pyrimidin-4(3H)-one (HCOOH salt, 2.37 mg, 7.6%, 8·HCOOH) (C₃₂H₃₆C₁N₇O₃·HCOOH).

LCMS (ESI, m/z): [M+1]⁺=602; RT=1.709 min.

¹H NMR (400 MHz, DMSO-d₆) δ 8.26 (d, J=8.8 Hz, 1H), 8.30 (s, 1H), 8.15 (d, J=8.0 Hz, 1H), 7.90-7.65 (m, 3H), 7.60 (t, J=7.8 Hz, 1H), 6.95-6.61 (m, 1H), 6.13 (d, J=15.6 Hz, 1H), 5.69 (d, J=9.6 Hz, 1H), 4.82 (s, 1H), 4.57-4.05 (m, 4H), 3.29 (s, 3H), 2.98-2.92 (m, 1H), 2.63-2.53 (m, 1H), 2.35 (d, J=1.2 Hz, 3H), 2.24-2.14 (m, 1H), 2.12-1.87 (m, 4H), 1.76-1.53 (m, 3H), 1.48-1.27 (m, 3H), 1.18 (s, 3H).

### Example 9

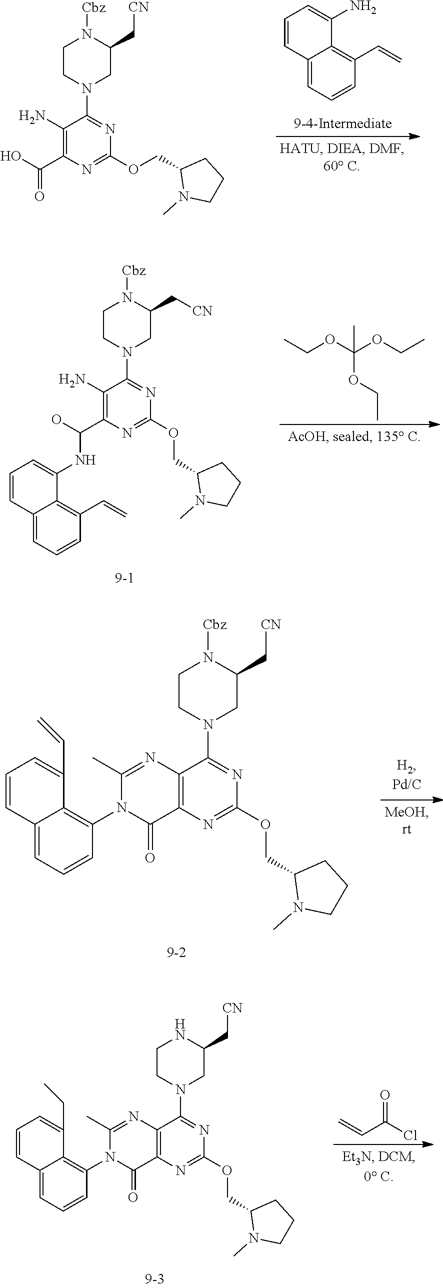

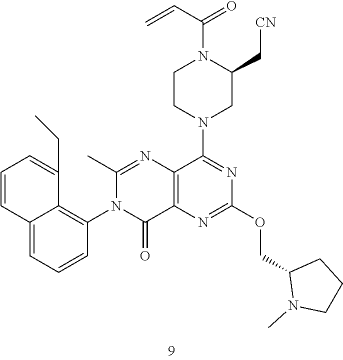 [SMILES: C=CC(=O)N1CCN(c2nc(OC[C@@H]3CCCN3C)nc3c(=O)n(-c4cccc5cccc(CC)c45)c(C)nc23)C[C@@H]1CC#N]

### Step 1: Synthesis of Compound 9-2-Intermediate

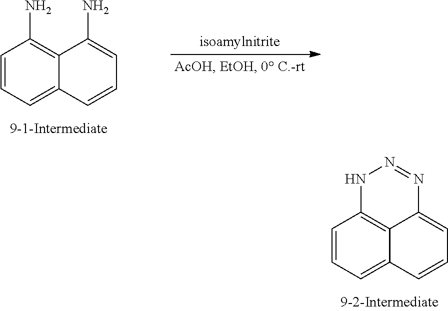 [SMILES: Nc1cccc2cccc(N)c12 ; c1cc2c3c(cccc3c1)NN=N2]

To a cooled (0° C.) solution of naphthalene-1,8-diamine (20 g, 126.58 mmol, 1.0 eq.) in EtOH (400 mL) and AcOH (40 mL) was added dropwise isoamylnitrite (16.6 mL, 124.05 mmol, 0.98 eq.). After addition, the reaction mixture was stirred at room temperature for overnight. LCMS analysis showed starting material was consumed and desired product formed. The solid was collected by filtration, washed with EtOH (200 mL) and dried under vacuum to obtain 1H-naphtho[1,8-de][1,2,3]triazine (18 g, 86%), which was used directly for the next step.

LCMS (ESI, m/z): [M+1]⁺=170; RT=1.219 min.

### Synthesis of Compound 9-3-Intermediate

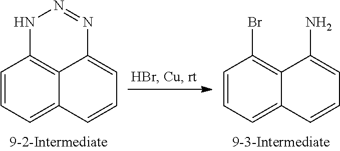 [SMILES: c1cc2c3c(cccc3c1)NN=N2 ; Nc1cccc2cccc(Br)c12]

To a cooled (0° C.) mixture of copper turnings (0.5 g, 7.81 mmol, 0.07 eq.) in aq.HBr (48%, 200 mL) was added slowly 1H-naphtho[1,8-de][1,2,3]triazine (18 g, 106.51 mmol, 1.0 eq.). After addition, the reaction mixture was stirred at room temperature for overnight. LCMS analysis showed starting material was consumed and desired product formed. The reaction mixture was diluted with EtOAc (50 mL), followed by the addition of aq. KOH (45w %) to adjust pH=11-12. The organic layer of the filtrate was separated and the aqueous layer was extracted with EtOAc (50 mL×2). The combined organics were dried over anhydrous Na₂SO₄ and concentrated to obtain 8-bromonaphthalen-1-amine (15.8 g, 67%), which was used directly for the next step.

LCMS (ESI, m/z): [M+1]⁺=222; RT=1.575 min.

### Step 3: Synthesis of Compound 9-4-Intermediate

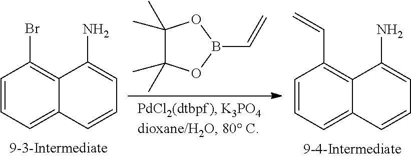

To a solution of 8-bromonaphthalen-1-amine (1 g, 4.52 mmol, 1.0 eq.) and 4,4,5,5-tetramethyl-2-vinyl-1,3,2-dioxaborolane (1.39 g, 9.04 mmol, 2.0 eq.) in dioxane (20 mL) and H₂O (5 mL) was added PdCl₂(dtbpf) (0.296 g, 0.45 mmol, 0.1 eq.) and K₃PO₄ (2.88 g, 13.38 mmol, 3.0 eq.). The mixture was stirred at 80° C. under Ar for overnight. LCMS analysis showed starting material was consumed and desired product formed. The reaction mixture was filtered through celite and the filtrate was concentrated to dryness. The residue was purified by silica column chromatography eluting with EtOAc/Pet.ether (10%, v/v) to obtain 8-vinylnaphthalen-1-amine (500 mg, 65%, 9-4-Intermediate).

LCMS (ESI, m/z): [M+1]⁺=170; RT=1.790 min.

### Step 4: Synthesis of Compound 9-1

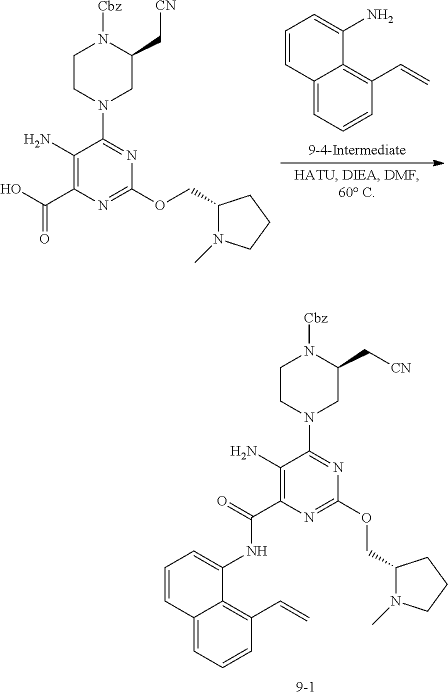 [SMILES: CN1CCC[C@H]1COc1nc(C(=O)O)c(N)c(N2CCN(C(=O)OCc3ccccc3)[C@@H](CC#N)C2)n1 ; C=Cc1cccc2cccc(N)c12 ; C=Cc1cccc2cccc(NC(=O)c3nc(OC[C@@H]4CCCN4C)nc(N4CCN(C(=O)OCc5ccccc5)[C@@H](CC#N)C4)c3N)c12]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (300 mg, 0.589 mmol, 1.0 eq.) and 8-vinylnaphthalen-1-amine (99 mg, 0.589 mmol, 1.0 eq.) in anhydrous DMF (4.0 mL) was added DIEA (0.29 mL, 1.768 mmol, 3.0 eq.), followed by the addition of HATU (223 mg, 0.589 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄ and concentrated. The residue was purified by silica column chromatography eluting with DCM/MeOH (15:1, v/v) to obtain benzyl (S)-4-(5-amino-2-(((S)-1-methylpyrrolidin-2-yl)methoxy)-6-((8-vinylnaphthalen-1-yl)carbamoyl)pyrimidin-4-yl)-2-(cyanomethyl)piperazine-1-carboxylate (210 mg, 54%).

LCMS (ESI, m/z): [M+1]⁺=661; RT=1.213 min.

### Step 5: Synthesis of Compound 9-2

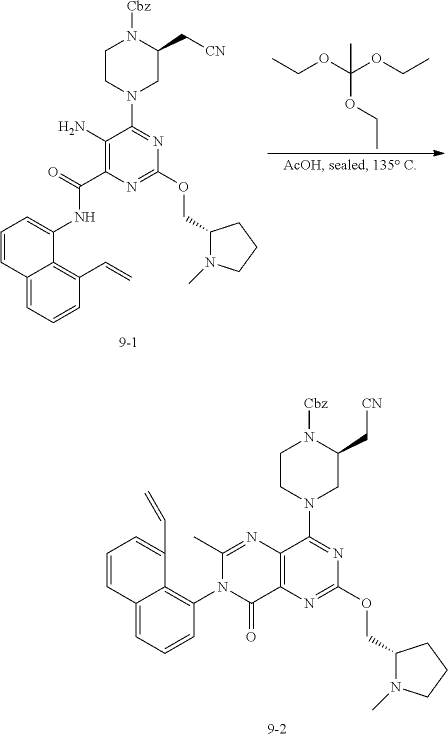 [SMILES: C=Cc1cccc2cccc(NC(=O)c3nc(OC[C@@H]4CCCN4C)nc(N4CCN(C(=O)OCc5ccccc5)[C@@H](CC#N)C4)c3N)c12 ; CCOC(C)(OCC)OCC ; C=Cc1cccc2cccc(-n3c(C)nc4c(N5CCN(C(=O)OCc6ccccc6)[C@@H](CC#N)C5)nc(OC[C@@H]5CCCN5C)nc4c3=O)c12]

To a mixture of benzyl (S)-4-(5-amino-2-(((S)-1-methylpyrrolidin-2-yl)methoxy)-6-((8-vinylnaphthalen-1-yl)carbamoyl)pyrimidin-4-yl)-2-(cyanomethyl)piperazine-1-carboxylate (200 mg, 0.303 mmol, 1.0 eq.) and AcOH (1.0 mL) was added 1,1,1-triethoxyethane (736 mg, 4.54 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 40 mL) to adjust pH=7-8, which was extracted with DCM (20 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(6-methyl-2-(((S)-1-methylpyrrolidin-2-yl)methoxy)-8-oxo-7-(8-vinylnaphthalen-1-yl)-7,8-dihydropyrimido[5,4-d]pyrimidin-4-yl)piperazine-1-carboxylate (110 mg, 53%).

LCMS (ESI, m/z): [M+1]⁺=685; RT=1.190 min.

### Step 6: Synthesis of Compound 9-3

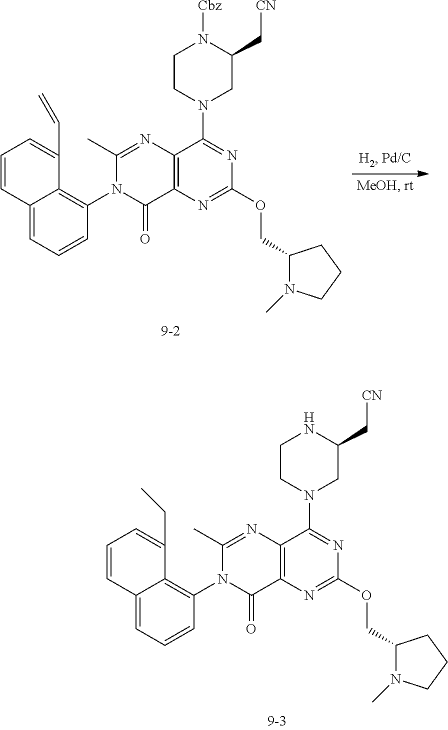 [SMILES: C=Cc1cccc2cccc(-n3c(C)nc4c(N5CCN(C(=O)OCc6ccccc6)[C@@H](CC#N)C5)nc(OC[C@@H]5CCCN5C)nc4c3=O)c12 ; CCc1cccc2cccc(-n3c(C)nc4c(N5CCN[C@@H](CC#N)C5)nc(OC[C@@H]5CCCN5C)nc4c3=O)c12]

To a solution of benzyl (S)-2-(cyanomethyl)-4-(6-methyl-2-(((S)-1-methylpyrrolidin-2-yl)methoxy)-8-oxo-7-(8-vinylnaphthalen-1-yl)-7,8-dihydropyrimido[5,4-d]pyrimidin-4-yl)piperazine-1-carboxylate (110 mg, 0.161 mmol) in MeOH (5.0 mL) was Pd/C (50 mg), and the mixture was stirred at room temperature for 1 h under H₂. LCMS showed starting material was consumed and desired product formed. The resulting mixture was filtered through celite. The organic layer of the filtrate was concentrated obtain 2-((S)-4-(7-(8-ethylnaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (73 mg, 82%), which was used directly for the next step.

### Step 7: Synthesis of Compound 9

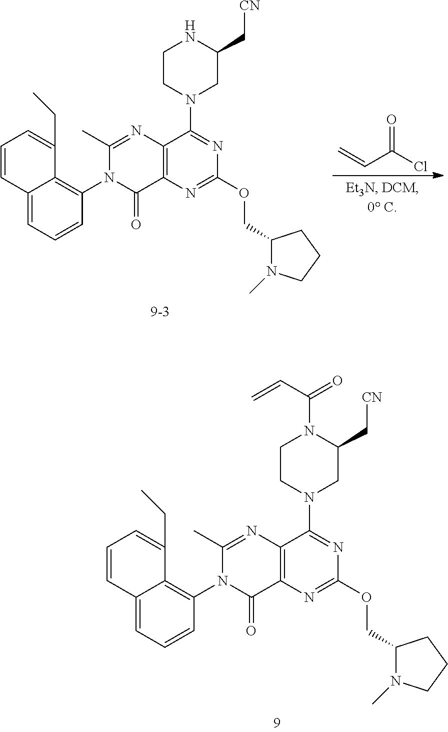

To a cooled (0° C.) solution of 2-((S)-4-(7-(8-ethylnaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (73 mg, 0.13 mmol, 1.0 eq.) and Et₃N (40 mg, 0.39 mmol, 3.0 eq.) in DCM (2.5 mL) was added dropwise a solution of acryloyl chloride (12 mg, 0.13 mmol, 1.0 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(8-ethylnaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (HCOOH salt, 20.5 mg, 25.6%, 9·HCOOH) (C₃₄H₃₈N₈O₃·HCOOH).

LCMS (ESI, m/z): [M+1]⁺=607; RT=1.020 min;

¹H NMR (400 MHz, DMSO-d₆) δ 8.31 (s, 1H), 8.15 (d, J=8.0 Hz, 1H), 7.98 (d, J=8.0 Hz, 1H), 7.66 (t, J=7.7 Hz, 1H), 7.60-7.52 (m, 2H), 7.45 (d, J=7.1 Hz, 1H), 6.95-6.79 (m, 1H), 6.20 (d, J=16.5 Hz, 1H), 5.78 (d, J=10.5 Hz, 1H), 4.35-4.13 (m, 6H), 3.44-2.82 (m, 6H), 2.63-2.51 (m, 3H), 2.36 (s, 3H), 2.23-2.12 (m, 1H), 2.04 (s, 3H), 1.98-1.87 (m, 1H), 1.74-1.54 (m, 3H), 1.06 (t, J=4 Hz, 3H).

### Example 10

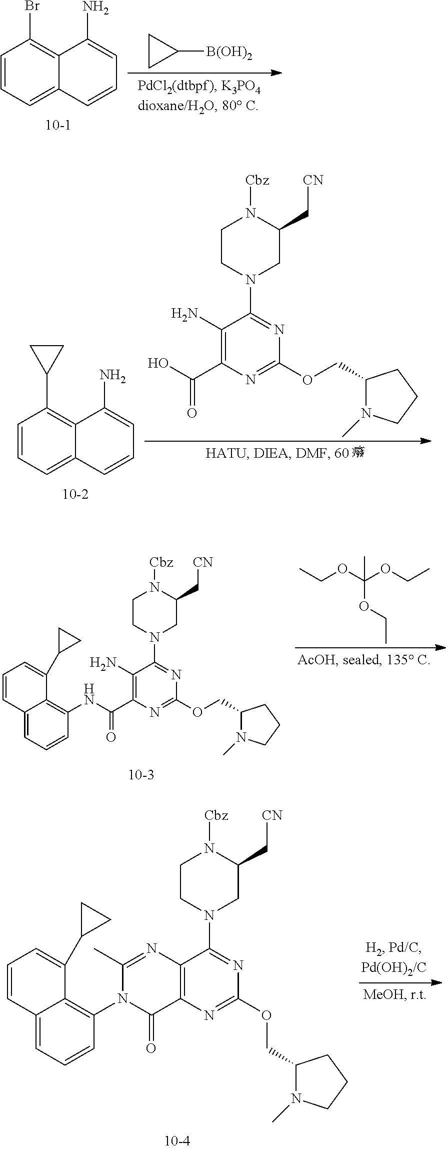

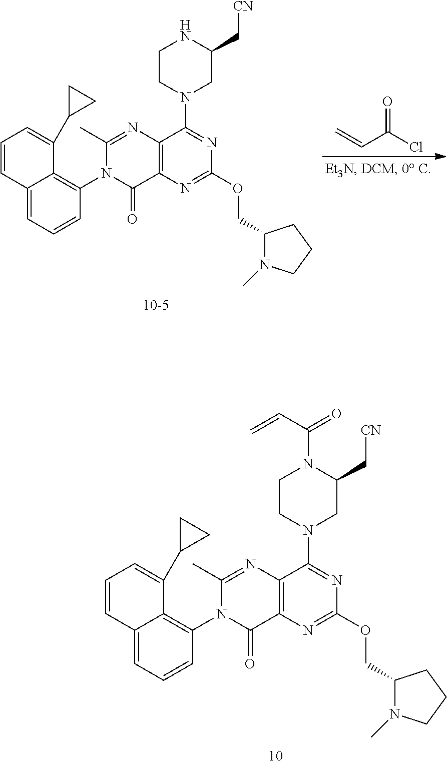

### Step 1: Synthesis of Compound 10-2

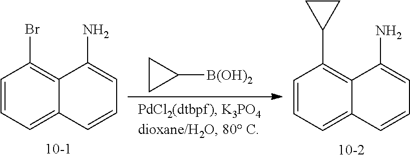

To a solution of 8-bromonaphthalen-1-amine (1) (1.00 g, 4.50 mmol, 1.0 eq.) and cyclopropylboronic acid (773 mg, 9.00 mmol, 2.0 eq.) in dioxane (40 mL) and H₂O (10 mL) was added PdCl₂(dtbpf) (293 mg, 0.45 mmol, 0.1 eq.) and K₃PO₄ (2.87 g, 13.5 mmol, 3.0 eq.). The mixture was stirred at 80° C. under Ar for 15 h. LCMS showed starting material was consumed and desired product formed. The reaction mixture was filtered through celite and the filtrate was concentrated to dryness. The residue was purified by silica column chromatography eluting with EtOAc/Pet.ether (10%, v/v) to obtain 8-cyclopropylnaphthalen-1-amine (380 mg, 46%).

LCMS (ESI, m/z): [M+1]⁺=184; RT=1.785 min.

### Step 2: Synthesis of Compound 10-3

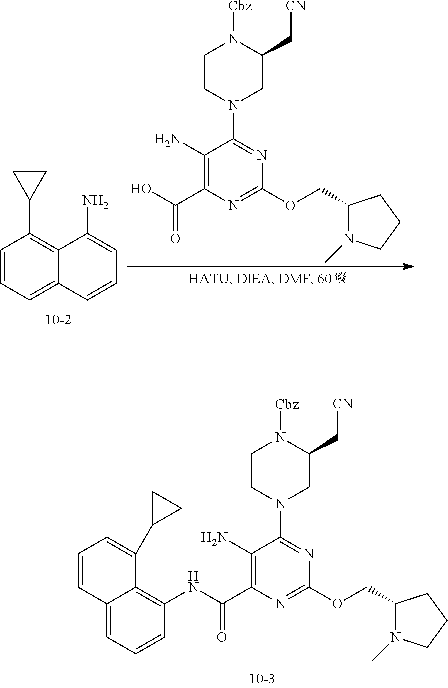 [SMILES: Nc1cccc2cccc(C3CC3)c12 ; CN1CCC[C@H]1COc1nc(C(=O)O)c(N)c(N2CCN(C(=O)OCc3ccccc3)[C@@H](CC#N)C2)n1 ; CN1CCC[C@H]1COc1nc(C(=O)Nc2cccc3cccc(C4CC4)c23)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (300 mg, 0.589 mmol, 1.0 eq.) and 8-cyclopropylnaphthalen-1-amine (76 mg, 0.412 mmol, 0.7 eq.) in anhydrous DMF (4.0 mL) was added DIEA (0.29 mL, 1.77 mmol, 3.0 eq.), followed by the addition of HATU (224 mg, 0.589 mmol, 1.0 eq.). The reaction mixture was stirred at 60° C. under Ar for 1 h. LCMS showed most of starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄ and concentrated. The residue was purified by silica column chromatography eluting with DCM/MeOH (15:1, v/v) to obtain benzyl (S)-4-(5-amino-6-((8-cyclopropylnaphthalen-1-yl)carbamoyl)-2-(((S)-1-methylpyrrolidin-2-yl)methoxy)pyrimidin-4-yl)-2-(cyanomethyl)piperazine-1-carboxylate (94 mg, 24%).

LCMS (ESI, m/z): [M+1]⁺=675; RT=1.254 min.

### Step 3: Synthesis of Compound 10-4

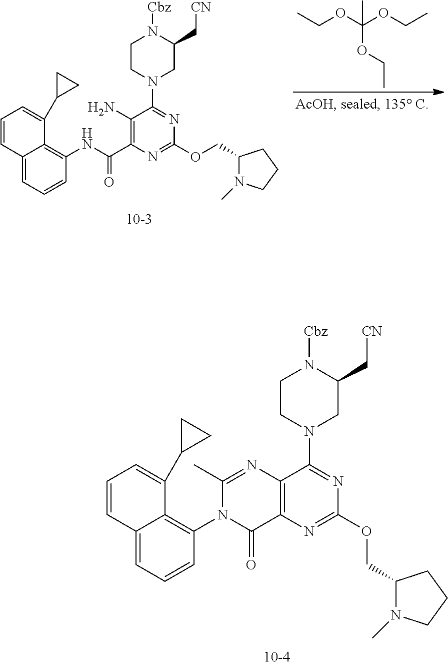 [SMILES: CN1CCC[C@H]1COc1nc(C(=O)Nc2cccc3cccc(C4CC4)c23)c(N)c(N2CCN(C(=O)OCc3ccccc3)[C@@H](CC#N)C2)n1 ; CCOC(C)(OCC)OCC ; Cc1nc2c(N3CCN(C(=O)OCc4ccccc4)[C@@H](CC#N)C3)nc(OC[C@@H]3CCCN3C)nc2c(=O)n1-c1cccc2cccc(C3CC3)c12]

To a mixture of benzyl (S)-4-(5-amino-6-((8-cyclopropylnaphthalen-1-yl)carbamoyl)-2-(((S)-1-methylpyrrolidin-2-yl)methoxy)pyrimidin-4-yl)-2-(cyanomethyl)piperazine-1-carboxylate (94 mg, 0.139 mmol, 1.0 eq.) and AcOH (1.0 mL) was added 1,1,1-triethoxyethane (372 mg, 2.09 mmol, 15.0 eq.). The mixture was stirred at 135° C. in a sealed tube for 7 min. LCMS showed starting material was consumed and desired product formed. The reaction mixture was quenched with aq. NaHCO₃ (sat. 40 mL) to adjust pH=7-8, which was extracted with DCM (20 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (56 mg, 57%).

LCMS (ESI, m/z): [M+1]⁺=699; RT=1.303 min.

### Step 4: Synthesis of Compound 10-5

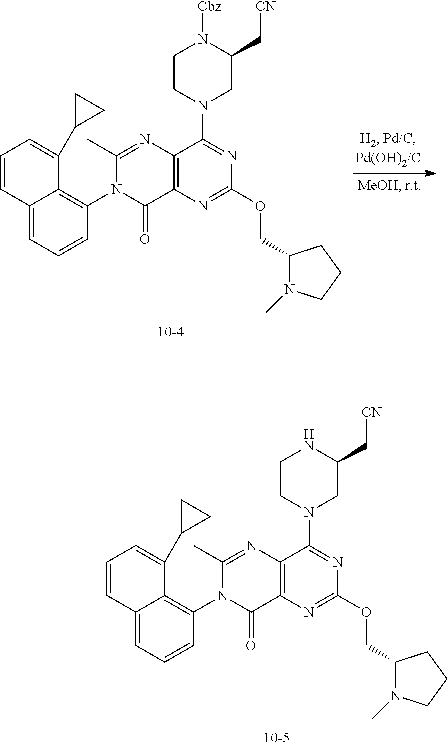 [SMILES: Cc1nc2c(N3CCN(C(=O)OCc4ccccc4)[C@@H](CC#N)C3)nc(OC[C@@H]3CCCN3C)nc2c(=O)n1-c1cccc2cccc(C3CC3)c12 ; Cc1nc2c(N3CCN[C@@H](CC#N)C3)nc(OC[C@@H]3CCCN3C)nc2c(=O)n1-c1cccc2cccc(C3CC3)c12]

To a solution of benzyl (S)-2-(cyanomethyl)-4-(6-methyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (56 mg, 0.080 mmol, 1.0 eq.) in MeOH (5.0 mL) was added Pd/C (10% w/w, 8.5 mg, 0.0080 mmol, 0.1 eq.) and Pd(OH)₂/C (10% w/w, 11 mg, 0.0080 mmol, 0.1 eq.). The reaction mixture was stirred at room temperature under H₂ (balloon) for 1 h. LCMS showed starting material was consumed and desired product formed. The mixture was filtered through celite and the filtrate was concentrated to dryness to obtain 2-((S)-4-(7-(8-cyclopropylnaphthalen-1-yl)-6-methyl-2-(((S)-1-methylpyrrolidin-2-yl)methoxy)-8-oxo-7,8-dihydropyrimido [5,4-d]pyrimidin-4-yl)piperazin-2-yl)acetonitrile (40 mg, 89%), which was used directly for the next step.

LCMS (ESI, m/z): [M+1]⁺=565; RT=0.409 min & 0.757 min.

### Step 5: Synthesis of Compound 10

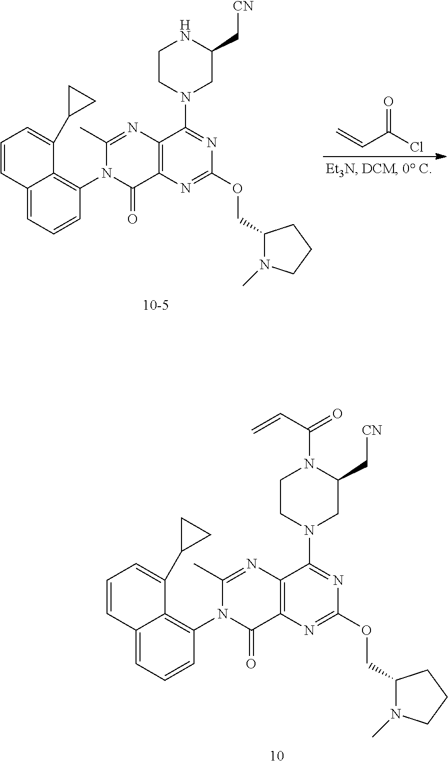

To a cooled (0° C.) solution of 2-((S)-4-(7-(8-cyclopropylnaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (40 mg, 0.071 mmol, 1.0 eq.) and Et₃N (36 mg, 0.354 mmol, 5.0 eq.) in DCM (3.0 mL) was added dropwise a solution of acryloyl chloride (7.7 mg, 0.085 mmol, 1.2 eq.) in DCM (0.5 mL). After addition, the mixture was stirred at 0° C. for 30 min. LCMS showed starting material was consumed and desired product formed. Water (10 mL) was added and the organic layer was separated. The aqueous layer was extracted with DCM (5 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(8-cyclopropylnaphthalen-1-yl)-6-methyl-2-(((S)-1-methylpyrrolidin-2-yl)methoxy)-8-oxo-7,8-dihydropyrimido [5,4-d]pyrimidin-4-yl)piperazin-2-yl)acetonitrile (HCOOH salt, 18.64 mg, 39%, 10·HCOOH) (C₃₅H₃₈N₈O₃·HCOOH).

LCMS (ESI, m/z): [M+1]⁺=619; RT=1.623 min.

¹H NMR (400 MHz, DMSO-d₆) δ 8.26 (s, 1H), 8.15 (d, J=8.0 Hz, 1H), 7.99 (d, J=8.0 Hz, 1H), 7.68 (t, J=7.6 Hz, 1H), 7.57 (t, J=6.0 Hz, 1H), 7.51 (t, J=7.8 Hz, 1H), 7.42 (d, J=7.2 Hz, 1H), 6.92-6.82 (m, 1H), 6.20 (dd, J=16.6, 2.2 Hz, 1H), 5.78 (d, J=10.4 Hz, 1H), 5.04-4.81 (m, 2H), 4.48 (s, 1H), 4.35-4.28 (m, 1H), 4.17-4.11 (m, 1H), 3.20-3.09 (s, 4H), 3.02-2.90 (m, 3H), 2.59-2.54 (m, 1H), 2.35 (d, J=2.0 Hz, 3H), 2.18 (dd, J=17.0, 8.6 Hz, 1H), 2.08 (s, 3H), 1.98-1.90 (m, 1H), 1.80-1.57 (m, 4H), 0.81-0.71 (m, 1H), 0.63-0.56 (m, 1H), 0.47 (t, J=7.8 Hz, 2H).

### Example 11

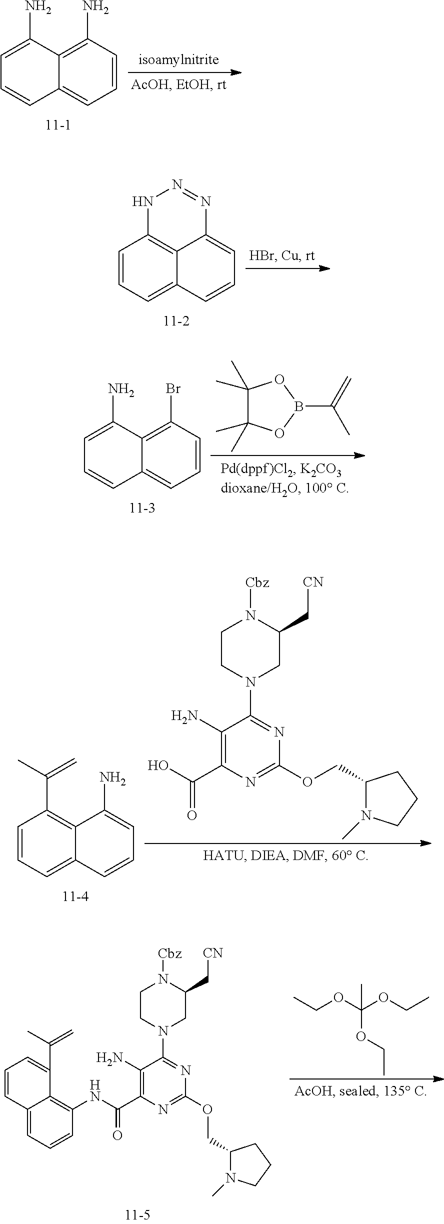

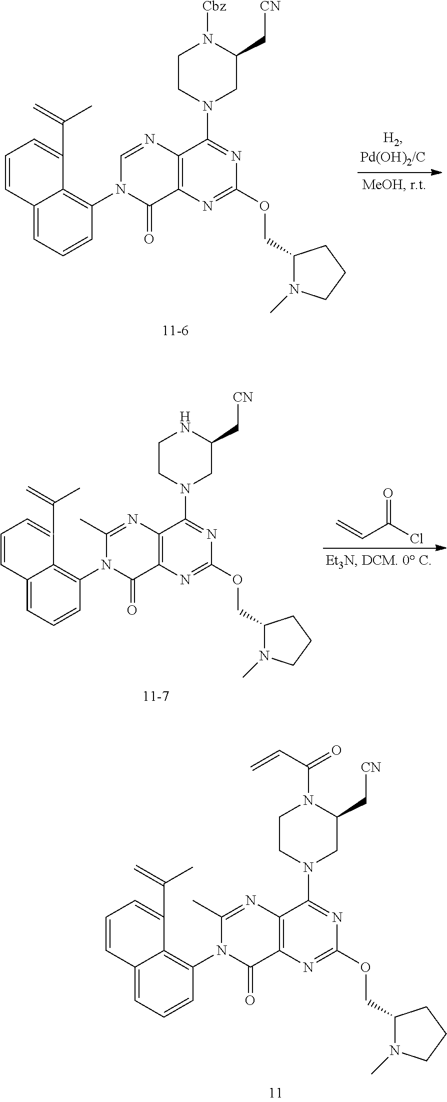

### Step 1: Synthesis of Compound 11-2

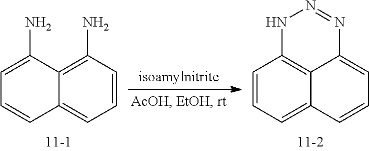 [SMILES: Nc1cccc2cccc(N)c12 ; c1cc2c3c(cccc3c1)NN=N2]

To a cooled (0° C.) solution of naphthalene-1,8-diamine (20 g, 126.58 mmol, 1.0 eq.) in EtOH (400 mL) and AcOH (40 mL) was added dropwise isoamylnitrite (16.6 mL, 124.05 mmol, 0.98 eq.). After addition, the reaction mixture was stirred at room temperature for overnight. LCMS analysis showed starting material was consumed and desired product formed. The solid was collected by filtration, washed with EtOH (200 mL) and dried under vacuum to obtain 1H-naphtho[1,8-de][1,2,3]triazine (18 g, 86%), which was used directly for the next step.

LCMS (ESI, m/z): [M+1]⁺=170; RT=1.219 min.

### Step 2: Synthesis of Compound 11-3

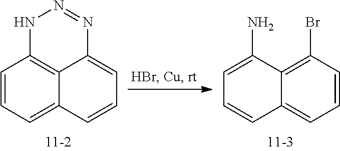 [SMILES: c1cc2c3c(cccc3c1)NN=N2 ; Nc1cccc2cccc(Br)c12]

To a cooled (0° C.) mixture of copper turnings (0.5 g, 7.81 mmol, 0.07 eq.) in aq.HBr (48%, 200 mL) was added slowly 1H-naphtho[1,8-de][1,2,3]triazine (18 g, 106.51 mmol, 1.0 eq.). After addition, the reaction mixture was stirred at room temperature for overnight. LCMS analysis showed starting material was consumed and desired product formed. The reaction mixture was diluted with EtOAc (50 mL), followed by the addition of aq. KOH (45%, w/w) to adjust pH=11-12. The organic layer of the filtrate was separated and the aqueous layer was extracted with EtOAc (50 mL×2). The combined organics were dried over anhydrous Na₂SO₄ and concentrated to obtain 8-bromonaphthalen-1-amine (15.8 g, 67%), which was used directly for the next step.

LCMS (ESI, m/z): [M+1]⁺=222; RT=1.575 min.

### Step 3: Synthesis of Compound 11-4

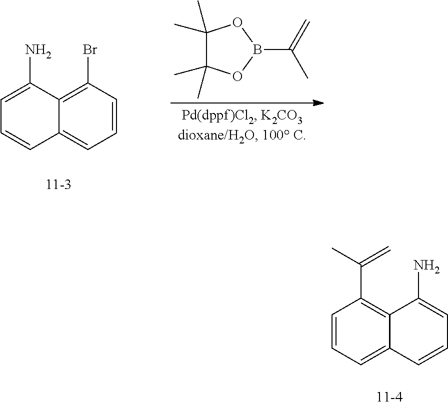

To a solution of 8-bromonaphthalen-1-amine (1 g, 4.52 mmol, 1.0 eq.) and 4,4,5,5-tetramethyl-2-(prop-1-en-2-yl)-1,3,2-dioxaborolane (1.14 g, 6.79 mmol, 1.5 eq.) in dioxane (10 mL) and H₂O (2 mL) was added Pd (dppf) Cl₂ (0.33 g, 0.45 mmol, 0.1 eq.) and K₂CO₃ (1.88 g, 13.56 mmol, 3.0 eq.). The mixture was stirred at 100° C. under Ar for overnight. LCMS analysis showed starting material was consumed and desired product formed. The reaction mixture was filtered through celite and the filtrate was concentrated to dryness. The residue was purified by silica column chromatography eluting with EtOAc/Pet.ether (10%, v/v) to obtain 8-(prop-1-en-2-yl)naphthalen-1-amine (592 mg, 69%).

LCMS (ESI, m/z): [M+1]⁺=184; RT=1.726 min.

### Step 4: Synthesis of Compound 11-5

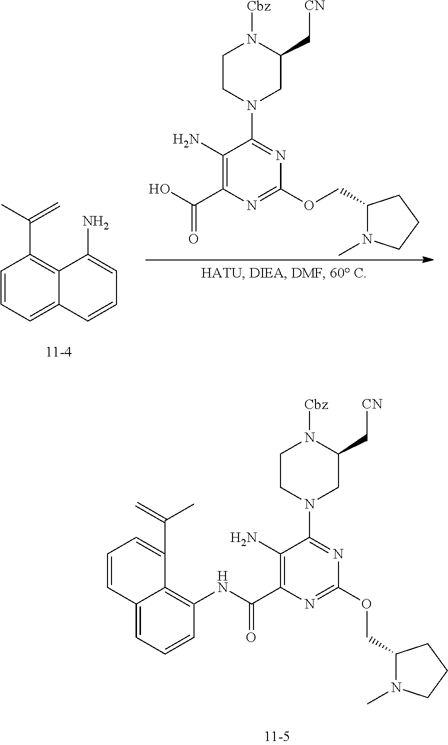 [SMILES: C=C(C)c1cccc2cccc(N)c12 ; CN1CCC[C@H]1COc1nc(C(=O)O)c(N)c(N2CCN(C(=O)OCc3ccccc3)[C@@H](CC#N)C2)n1 ; C=C(C)c1cccc2cccc(NC(=O)c3nc(OC[C@@H]4CCCN4C)nc(N4CCN(C(=O)OCc5ccccc5)[C@@H](CC#N)C4)c3N)c12]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (200 mg, 0.392 mmol, 1.0 eq.) and 8-(prop-1-en-2-yl)naphthalen-1-amine (71.9 mg, 0.392 mmol, 1.0 eq.) in anhydrous DMF (4 mL) was added DIEA (0.37 mL, 1.18 mmol, 3.0 eq.), followed by the addition of HATU (149 mg, 0.392 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄ and concentrated. The residue was purified by silica column chromatography eluting with DCM/MeOH (10:1, v/v) to obtain benzyl (S)-4-(5-amino-2-(((S)-1-methylpyrrolidin-2-yl)methoxy)-6-((8-(prop-1-en-2-yl)naphthalen-1-yl)carbamoyl)pyrimidin-4-yl)-2-(cyanomethyl)piperazine-1-carboxylate (251 mg, 95%).

LCMS (ESI, m/z): [M+1]⁺=675; RT=1.265 min.

### Step 5: Synthesis of Compound 11-6

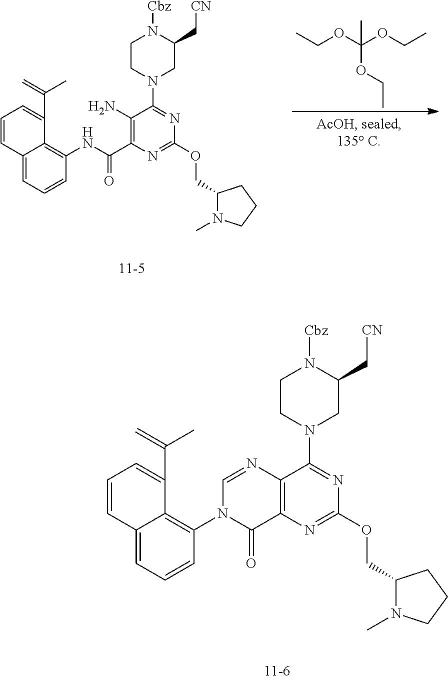 [SMILES: C=C(C)c1cccc2cccc(NC(=O)c3nc(OC[C@@H]4CCCN4C)nc(N4CCN(C(=O)OCc5ccccc5)[C@@H](CC#N)C4)c3N)c12 ; CCOC(C)(OCC)OCC ; C=C(C)c1cccc2cccc(-n3cnc4c(N5CCN(C(=O)OCc6ccccc6)[C@@H](CC#N)C5)nc(OC[C@@H]5CCCN5C)nc4c3=O)c12]

To a mixture of (S)-4-(5-amino-2-(((S)-1-methylpyrrolidin-2-yl)methoxy)-6-((8-(prop-1-en-2-yl)naphthalen-1-yl)carbamoyl)pyrimidin-4-yl)-2-(cyanomethyl)piperazine-1-carboxylate (299.3 mg, 0.44 mmol, 1.0 eq.) and AcOH (3.0 mL) was added 1,1,1-triethoxyethane (1.08 g, 6.66 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 30 mL) to adjust pH=7-8, which was extracted with DCM (15 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(6-methyl-2-(((S)-1-methylpyrrolidin-2-yl)methoxy)-8-oxo-7-(8-(prop-1-en-2-yl)naphthalen-1-yl)-7,8-dihydropyrimido[5,4-d]pyrimidin-4-yl)piperazine-1-carboxylate (91 mg, 29.6%).

LCMS (ESI, m/z): [M+1]⁺=699; RT=1.248 min.

### Step 6: Synthesis of Compound 11-7

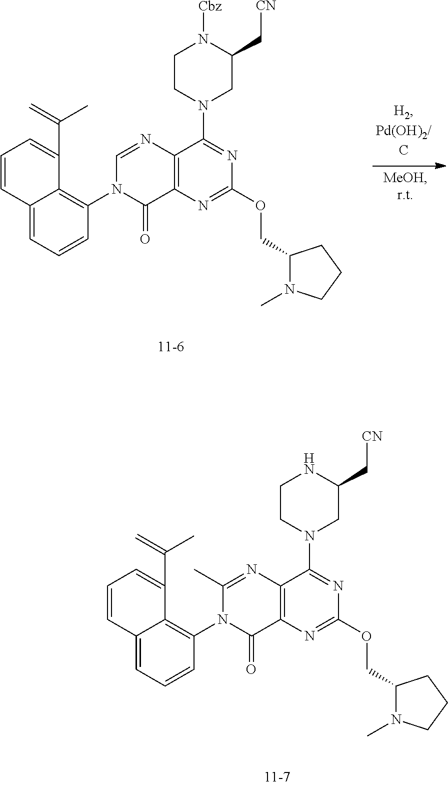 [SMILES: C=C(C)c1cccc2cccc(-n3cnc4c(N5CCN(C(=O)OCc6ccccc6)[C@@H](CC#N)C5)nc(OC[C@@H]5CCCN5C)nc4c3=O)c12 ; C=C(C)c1cccc2cccc(-n3c(C)nc4c(N5CCN[C@@H](CC#N)C5)nc(OC[C@@H]5CCCN5C)nc4c3=O)c12]

To a solution of benzyl (S)-2-(cyanomethyl)-4-(6-methyl-2-(((S)-1-methylpyrrolidin-2-yl)methoxy)-8-oxo-7-(8-(prop-1-en-2-yl)naphthalen-1-yl)-7,8-dihydropyrimido[5,4-d]pyrimidin-4-yl)piperazine-1-carboxylate (45 mg, 0.064 mmol, 1.0 eq.) in MeOH (3.0 mL) was added Pd(OH)₂/C (20% on carbon, wetted with ca.50% water, 4.23 mg, 0.006 mmol, 0.1 eq.), and the mixture was stirred at room temperature for 1 h under H₂. LCMS showed starting material was consumed and desired product formed. The reaction mixture was filtered and the filtrate was concentrated to dryness to obtain 2-((S)-4-(6-methyl-2-(((S)-1-methylpyrrolidin-2-yl)methoxy)-8-oxo-7-(8-(prop-1-en-2-yl)naphthalen-1-yl)-7,8-dihydropyrimido[5,4-d]pyrimidin-4-yl)piperazin-2-yl)acetonitrile (36 mg, 99%), which was used directly for the next step.

LCMS (ESI, m/z): [M+1]⁺=565; RT=0.728 min;

### Step 7: Synthesis of Compound 11

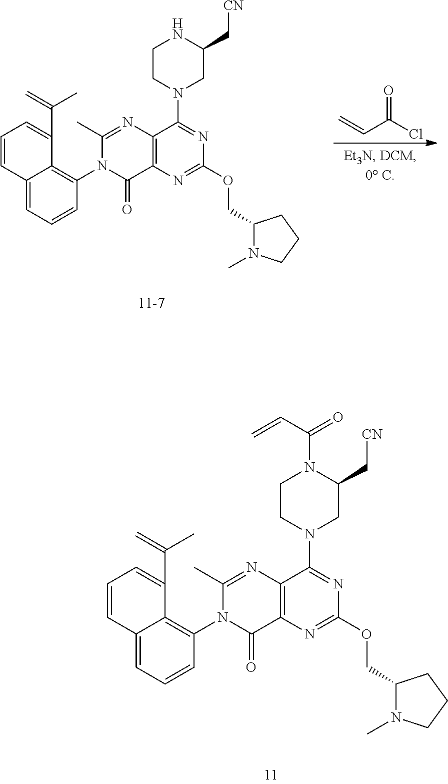

To a cooled (0° C.) solution (36.4 mg, 0.065 mmol, 1.0 eq.) and Et₃N (19.6 mg, 0.194 mmol, 3.0 eq.) in DCM (3.0 mL) was added dropwise a solution of acryloyl chloride (758 mg, 0.095 mmol, 1 eq.) in DCM (3 mL). After addition, the mixture was stirred at 0° C. for 30 min. LCMS showed starting material was consumed and desired product formed. Water (20 mL) was added and the organic layer was separated. The aqueous layer was extracted with DCM (10 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(6-methyl-2-(((S)-1-methylpyrrolidin-2-yl)methoxy)-8-oxo-7-(8-(prop-1-en-2-yl)naphthalen-1-yl)-7,8-dihydropyrimido[5,4-d]pyrimidin-4-yl)piperazin-2-yl)acetonitrile (HCOOH salt, 2.21 mg, 5.5%, 11·HCOOH) (C₃₅H₃₈N₈O₃·HCOOH).

LCMS (ESI, m/z): [M+1]⁺=619; RT=1.603 min;

¹H NMR (400 MHz, CDCl₃) δ 8.35 (s, 1H), 8.04 (d, J=8.0 Hz, 1H), 7.89 (d, J=8.0 Hz, 1H), 7.68-7.55 (m, 1H), 7.54-7.42 (m, 1H), 7.42-7.27 (m, 2H), 6.60 (d, J=11.6 Hz, 1H), 6.41 (d, J=16.4 Hz, 1H), 5.83 (d, J=10.4 Hz, 1H), 5.09 (s, 1H), 4.89 (s, 1H), 4.83-4.63 (m, 3H), 4.18-3.07 (m, 5H), 2.96 (s, 5H), 2.76 (d, J=15.6 Hz, 1H), 2.29 (s, 2H), 2.18-2.05 (m, 4H), 2.02-1.88 (m, 4H), 1.80 (d, J=2.8 Hz, 3H).

### Example 12

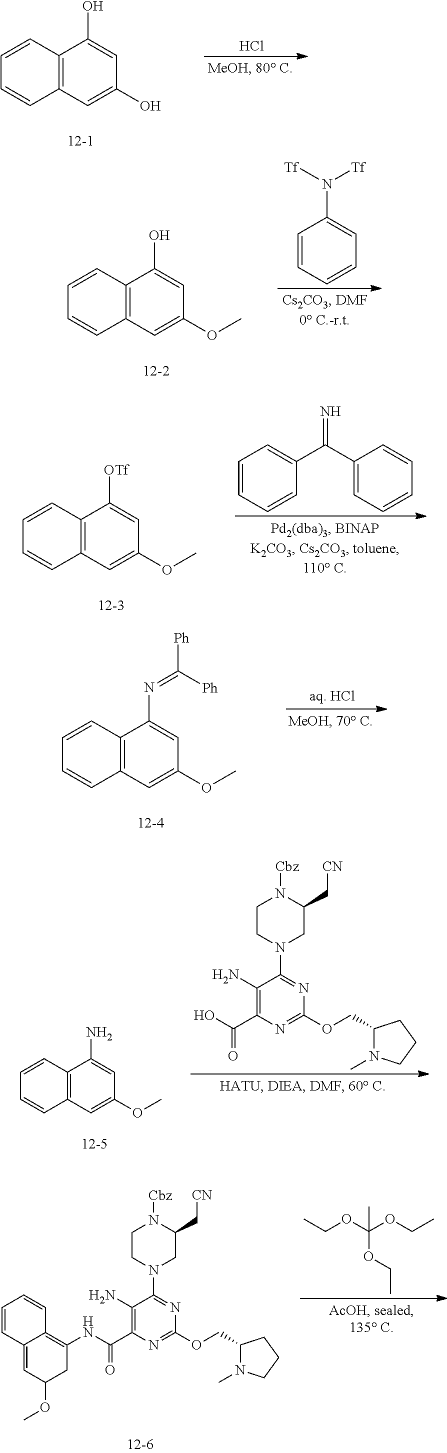

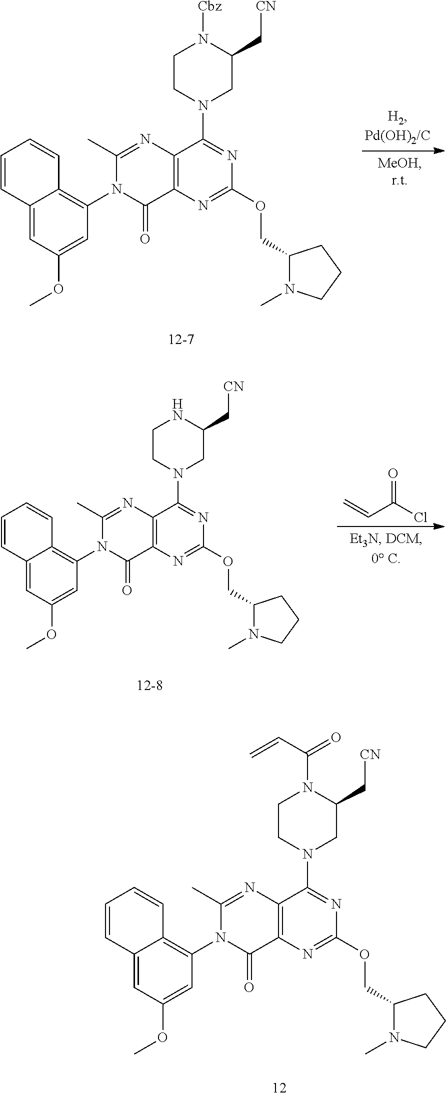

### Step 1: Synthesis of Compound 12-2

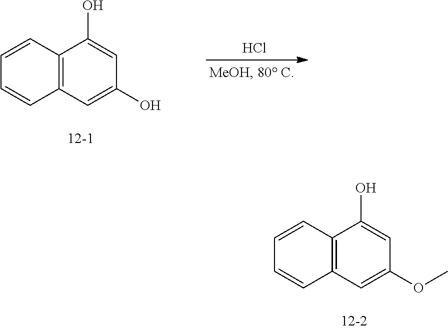

To a solution of naphthalene-1,3-diol (10.0 g, 62.5 mmol, 1.0 eq.) in MeOH (120 mL) was added conc.HCl (4.0 mL, 62.5 mmol, 0.76 eq.). The reaction mixture was stirred at 80° C. under Ar for 16 h. TLC showed starting material was consumed and new product formed. The reaction mixture was cooled to room temperature, diluted with NaHCO₃ (60 mL) and extracted with DCM (60 mL×3). The combined organic fractions were combined, dried over anhydrous Na₂SO₄ and concentrated. The residue was purified by silica column chromatography eluting with Pet.ether/EtOAc (4:1, v/v) to obtain 3-methoxynaphthalen-1-ol (7.2 g, 66.7%).

### Step 2: Synthesis of Compound 12-3

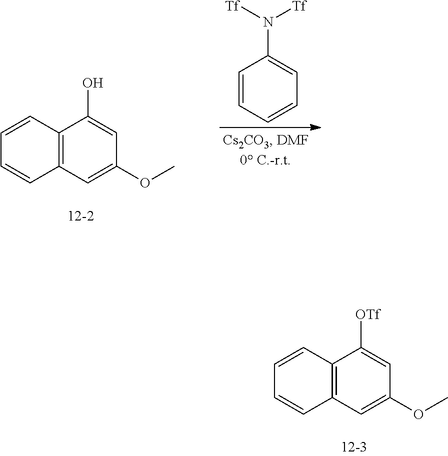

To a solution of 3-methoxynaphthalen-1-ol (500 mg, 2.87 mmol, 1.0 eq.) in anhydrous DMF (5.0 mL) was added CsCO₃ (1.87 g, 5.74 mmol, 2.0 eq.) and 1,1,1-trifluoro-N-phenyl-N-((trifluoromethyl)sulfonyl)methanesulfonamide (1.34 g, 3.74 mmol, 1.3 eq.). The reaction mixture was stirred at 0° C. under Ar for 6 h. TLC showed starting material was consumed and new product formed. The reaction mixture was cooled to room temperature, diluted with EtOAc (20 mL×3). The combined organic fractions were washed with brine (25 mL×3), dried over anhydrous Na₂SO₄ and concentrated. The residue was purified by silica column chromatography eluting with Pet.ether/EtOAc (4:1, v/v) to obtain 3-methoxynaphthalen-1-yl trifluoromethanesulfonate (689 mg, 78.4%).

### Step 3: Synthesis of Compound 12-4

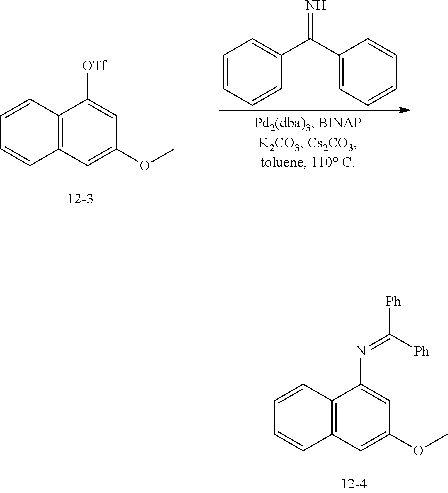 [SMILES: COc1cc(OS(=O)(=O)C(F)(F)F)c2ccccc2c1 ; N=C(c1ccccc1)c1ccccc1 ; COc1cc(N=C(c2ccccc2)c2ccccc2)c2ccccc2c1]

To a solution of 3-methoxynaphthalen-1-yl trifluoromethanesulfonate (389 mg, 1.27 mmol, 1.0 eq.) and benzophenone imine (299.1 mg, 1.65 mmol, 1.3 eq.) in toluene (4.0 mL) was added K₂CO₃ (228.1 mg, 1.65 mmol, 1.3 eq.), Cs₂CO₃ (538.7 mg, 1.65 mmol, 1.3 eq.) and BINAP (102.6 mg, 0.165 mmol, 0.1 eq.) followed by the addition of Pd₂ (dba)₃ (75.6 mg, 0.083 mmol, 0.05 eq.), and the mixture was stirred at 110° C. for 3 h under Ar. LCMS showed starting material was consumed and desired product formed. The reaction mixture was filtered and the filtrate was concentrated. The residue was purified by silica column chromatography eluting with Pet.ether/EtOAc (10:1, v/v) to obtain N-(3-methoxynaphthalen-1-yl)-1,1-diphenylmethanimine (396 mg, 92%).

LCMS (ESI, m/z): [M+1]⁺=338; RT=2.321 min.

### Step 4: Synthesis of Compound 12-5

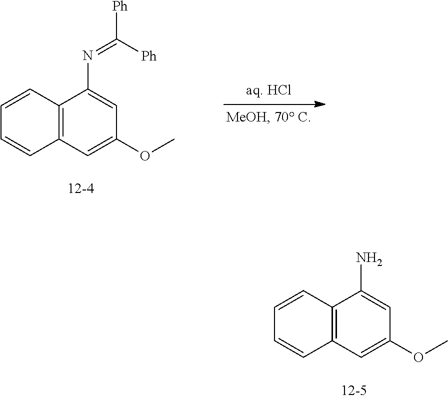 [SMILES: COc1cc(N=C(c2ccccc2)c2ccccc2)c2ccccc2c1 ; COc1cc(N)c2ccccc2c1]

To a mixture of N-(3-methoxynaphthalen-1-yl)-1,1-diphenylmethanimine (396 mg, 1.17 mmol) and MeOH (4.0 mL) and H₂O (4.0 mL) was added HCl (1.5 mL) dropwise. The mixture was stirred at 70° C. for 1.5 h. LCMS showed starting material was consumed and desired product formed. The reaction mixture was quenched with aq. NaHCO₃ (sat. 20 mL) to adjust pH=7-8, which was extracted with DCM (20 mL×3). The combined organic fractions were dried over anhydrous Na₂SO₄ and concentrated. The residue was purified by prep-TLC eluting with Pet.ether/EtOAc (10:1, v/v) to obtain 3-methoxynaphthalen-1-amine (193 mg, 95.5%).

LCMS (ESI, m/z): [M+1]⁺=174; RT=1.428 min.

### Step 5: Synthesis of Compound 12-6

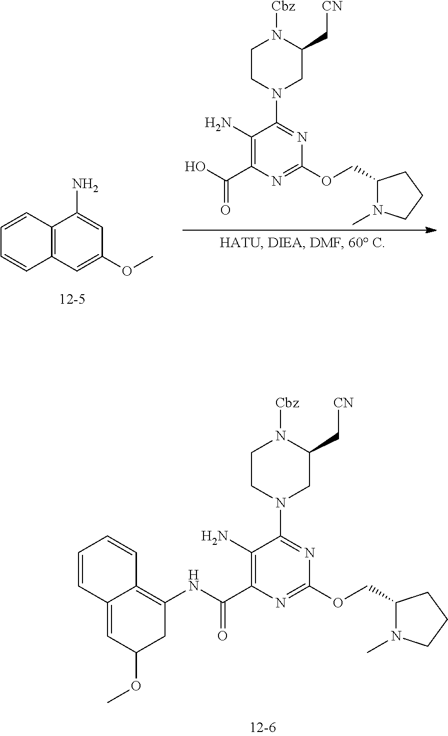 [SMILES: COc1cc(N)c2ccccc2c1 ; CN1CCC[C@H]1COc1nc(C(=O)O)c(N)c(N2CCN(C(=O)OCc3ccccc3)[C@@H](CC#N)C2)n1 ; COC1C=c2ccccc2=C(NC(=O)c2nc(OC[C@@H]3CCCN3C)nc(N3CCN(C(=O)OCc4ccccc4)[C@@H](CC#N)C3)c2N)C1]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (310 mg, 0.609 mmol, 1.0 eq.) and 3-methoxynaphthalen-1-amine (73.7 mg, 1.27 mmol, 0.7 eq.) in anhydrous DMF (5.0 mL) was added DIEA (0.30 mL, 1.827 mmol, 3.0 eq.), followed by the addition of HATU (232 mg, 0.609 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (25 mL), dried over anhydrous Na₂SO₄ and concentrated. The residue was purified by silica column chromatography eluting with DCM/MeOH (10:1, v/v) to obtain benzyl (S)-4-(5-amino-6-((3-methoxynaphthalen-1-yl)carbamoyl)-2-(((S)-1-methylpyrrolidin-2-yl)methoxy)pyrimidin-4-yl)-2-(cyanomethyl)piperazine-1-carboxylate (150 mg, 37.1%).

LCMS (ESI, m/z): [M+1]⁺=665; RT=1.218 min.

### Step 6: Synthesis of Compound 12-7

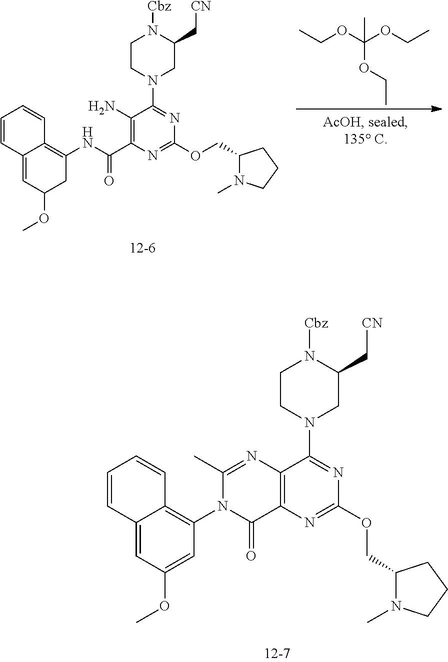 [SMILES: COC1C=c2ccccc2=C(NC(=O)c2nc(OC[C@@H]3CCCN3C)nc(N3CCN(C(=O)OCc4ccccc4)[C@@H](CC#N)C3)c2N)C1 ; CCOC(C)(OCC)OCC ; COc1cc(-n2c(C)nc3c(N4CCN(C(=O)OCc5ccccc5)[C@@H](CC#N)C4)nc(OC[C@@H]4CCCN4C)nc3c2=O)c2ccccc2c1]

To a mixture of benzyl (S)-4-(5-amino-6-((3-methoxynaphthalen-1-yl)carbamoyl)-2-(((S)-1-methylpyrrolidin-2-yl)methoxy)pyrimidin-4-yl)-2-(cyanomethyl)piperazine-1-carboxylate (153 mg, 0.23 mmol, 1.0 eq.) and AcOH (1.2 mL) was added 1,1,1-triethoxyethane (559 mg, 3.45 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 20 mL) to adjust pH=7-8, which was extracted with DCM (20 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(7-(3-methoxynaphthalen-1-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (126 mg, 79.7%).

LCMS (ESI, m/z): [M+1]⁺=689; RT=1.195 min.

### Step 7: Synthesis of Compound 12-8

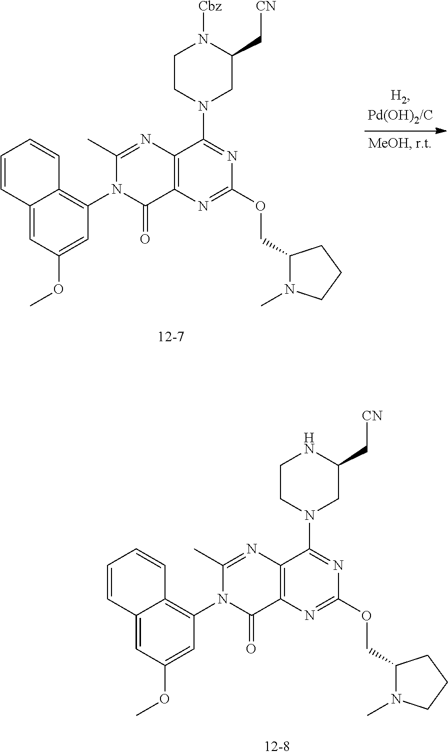 [SMILES: COc1cc(-n2c(C)nc3c(N4CCN(C(=O)OCc5ccccc5)[C@@H](CC#N)C4)nc(OC[C@@H]4CCCN4C)nc3c2=O)c2ccccc2c1 ; COc1cc(-n2c(C)nc3c(N4CCN[C@@H](CC#N)C4)nc(OC[C@@H]4CCCN4C)nc3c2=O)c2ccccc2c1]

To a solution of benzyl (S)-2-(cyanomethyl)-4-(7-(3-methoxynaphthalen-1-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (126 mg, 0.183 mmol, 1.0 eq.) in MeOH (8.0 mL) was added Pd(OH)₂/C (20% on carbon, wetted with ca.50% water, 12.6 mg, 0.018 mmol, 0.1 eq.), and the mixture was stirred at room temperature for 1 h under H₂. LCMS showed starting material was consumed and desired product formed. The reaction mixture was filtered and the filtrate was concentrated to dryness to obtain 2-((S)-4-(7-(3-methoxynaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (53 mg, 53%), which was used directly for the next step.

LCMS (ESI, m/z): [M+1]⁺=555; RT=0.349 min;

### Step 8: Synthesis of Compound 12

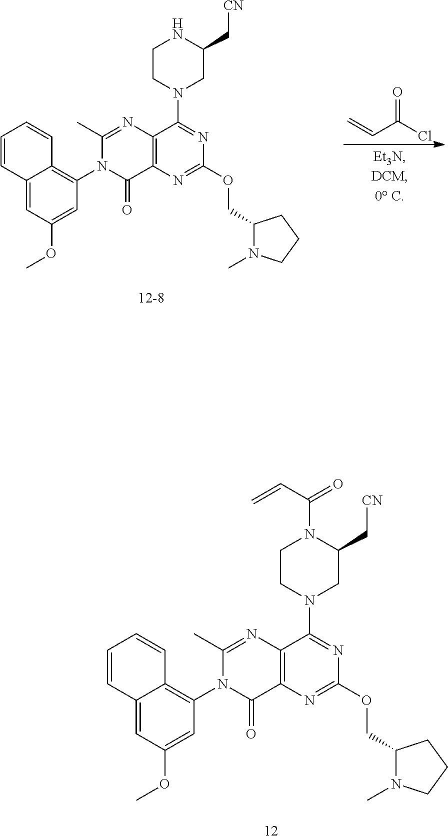

To a cooled (0° C.) solution of 2-((S)-4-(7-(3-methoxynaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (53 mg, 0.095 mmol, 1.0 eq.) and Et₃N (29 mg, 0.287 mmol, 3.0 eq.) in DCM (5 mL) was added dropwise a solution of acryloyl chloride (8.6 mg, 0.095 mmol, 1 eq.) in DCM (5.0 mL). After addition, the mixture was stirred at 0° C. for 30 min. LCMS showed starting material was consumed and desired product formed. Water (20 mL) was added and the organic layer was separated. The aqueous layer was extracted with DCM (10 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(3-methoxynaphthalen-1-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (HCOOH salt, 20.3 mg, 35.2%, 12·HCOOH) (C₃₃H₃₆N₈O₄·HCOOH).

LCMS (ESI, m/z): [M+1]⁺=609; RT=0.985 min;

¹H NMR (400 MHz, DMSO-d₆) δ 8.32 (s, 1H), 7.99 (d, J=8.0 Hz, 1H), 7.56 (d, J=8.4 Hz, 2H), 7.46-7.30 (m, 3H), 6.96-6.78 (m, 1H), 6.28-6.13 (m, 1H), 5.84-5.72 (m, 1H), 5.53-4.42 (m, 4H), 4.36-4.29 (m, 1H), 4.14 (dd, J=10.8, 5.6 Hz, 1H), 3.95 (s, 3H), 3.14 (s, 3H), 3.01-2.92 (m, 3H), 2.56 (s, 1H), 2.36-2.32 (m, 3H), 2.17 (dd, J=17.2, 8.6 Hz, 1H), 2.05 (s, 3H), 1.98-1.89 (m, 1H), 1.72-1.57 (m, 3H).

### Example 13

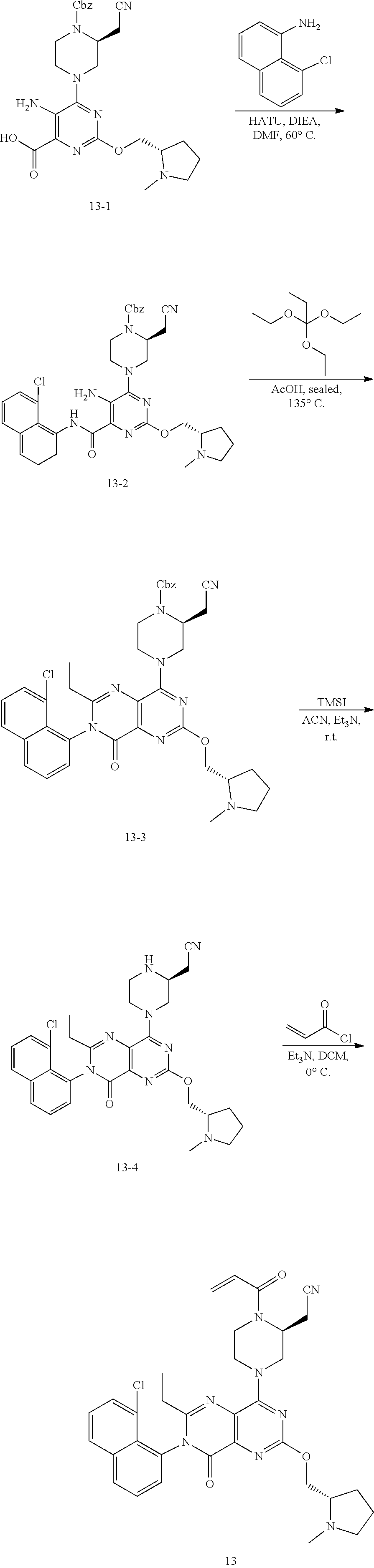

### Step 1: Synthesis of Compound 13-2

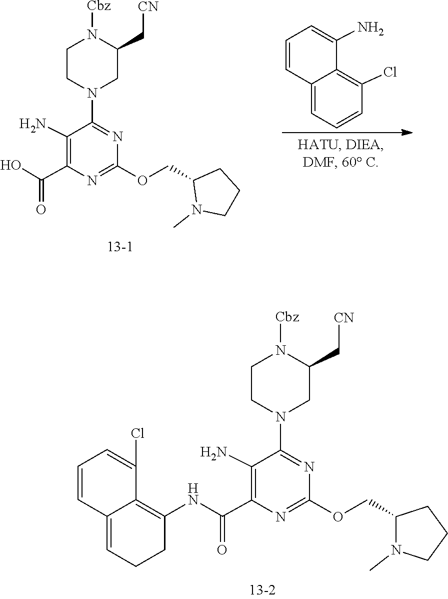 [SMILES: CN1CCC[C@H]1COc1nc(C(=O)O)c(N)c(N2CCN(C(=O)OCc3ccccc3)[C@@H](CC#N)C2)n1 ; Nc1cccc2cccc(Cl)c12 ; CN1CCC[C@H]1COc1nc(C(=O)NC2=c3c(Cl)cccc3=CCC2)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (500 mg, 0.981 mmol, 1.0 eq.) and 8-chloronaphthalen-1-amine (122 mg, 0.687 mmol, 0.7 eq.) in anhydrous DMF (5.0 mL) was added DIEA (0.81 mL, 4.91 mmol, 5.0 eq.), followed by the addition of HATU (373 mg, 0.981 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄ and concentrated. The residue was purified by silica column chromatography eluting with DCM/MeOH (15:1, v/v) to obtain benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (446 mg, 68%).

LCMS (ESI, m/z): [M+1]⁺=669; RT=1.226 min.

### Step 2: Synthesis of Compound 13-3

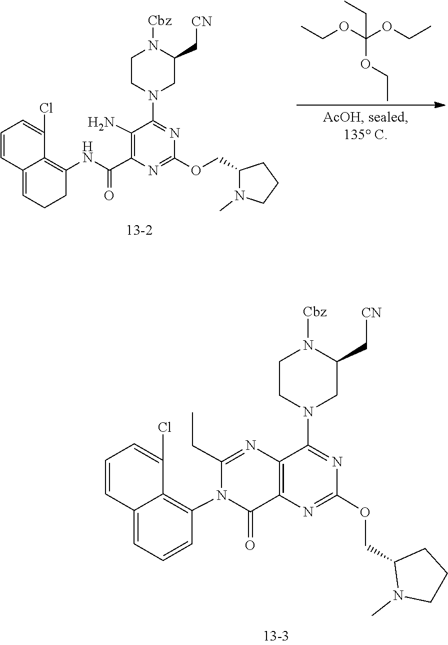 [SMILES: CN1CCC[C@H]1COc1nc(C(=O)NC2=c3c(Cl)cccc3=CCC2)c(N)c(N2CCN(C(=O)OCc3ccccc3)[C@@H](CC#N)C2)n1 ; CCOC(CC)(OCC)OCC ; CCc1nc2c(N3CCN(C(=O)OCc4ccccc4)[C@@H](CC#N)C3)nc(OC[C@@H]3CCCN3C)nc2c(=O)n1-c1cccc2cccc(Cl)c12]

To a mixture of benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (210 mg, 0.314 mmol, 1.0 eq.) and AcOH (2.0 mL) was added 1,1,1-triethoxypropane (830 mg, 4.71 mmol, 15.0 eq.). The mixture was stirred at 135° C. in a sealed tube for 7 min. LCMS showed starting material was consumed and desired product formed. The reaction mixture was quenched with aq. NaHCO₃ (sat. 90 mL) to adjust pH=7-8, which was extracted with DCM (30 mL×3). The combined organic fractions were dried over anhydrous Na₂SO₄ and concentrated. The residue was purified by prep-TLC eluting with DCM/MeOH (10:1, v/v) to obtain benzyl (S)-4-(7-(8-chloronaphthalen-1-yl)-6-ethyl-2-(((S)-1-methylpyrrolidin-2-yl)methoxy)-8-oxo-7,8-dihydropyrimido[5,4-d]pyrimidin-4-yl)-2-(cyanomethyl) piperazine-1-carboxylate (65 mg, 29%).

LCMS (ESI, m/z): [M+1]⁺=707; RT=1.303 min.

### Step 3: Synthesis of Compound 13-4

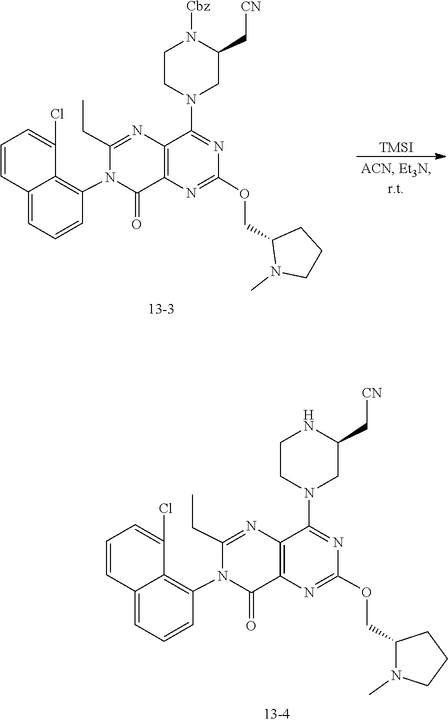 [SMILES: "TMSI" ; CCc1nc2c(N3CCN(C(=O)OCc4ccccc4)[C@@H](CC#N)C3)nc(OC[C@@H]3CCCN3C)nc2c(=O)n1-c1cccc2cccc(Cl)c12 ; CCc1nc2c(N3CCN[C@@H](CC#N)C3)nc(OC[C@@H]3CCCN3C)nc2c(=O)n1-c1cccc2cccc(Cl)c12]

To a solution of benzyl (S)-4-(7-(8-chloronaphthalen-1-yl)-6-ethyl-2-(((S)-1-methylpyrrolidin-2-yl)methoxy)-8-oxo-7,8-dihydropyrimido[5,4-d]pyrimidin-4-yl)-2-(cyanomethyl) piperazine-1-carboxylate (65 mg, 0.092 mmol, 1.0 eq.) in anhydrous ACN (5.0 mL) was added TMSI (184 mg, 0.920 mmol, 10.0 eq.). The reaction mixture was stirred at 30° C. under Ar for 1 h. LCMS showed most of starting material was consumed and desired product formed. Et₃N (0.5 mL, 3.60 mmol, 39.1 eq.) was added and the mixture was stirred at room temperature for 15 min. Then the mixture was concentrated and the residue was purified by prep-TLC eluting with DCM/MeOH (8:1, v/v) to obtain 2-((S)-4-(7-(8-chloronaphthalen-1-yl)-6-ethyl-2-(((S)-1-methylpyrrolidin-2-yl)methoxy)-8-oxo-7,8-dihydropyrimido[5,4-d]pyrimidin-4-yl)piperazin-2-yl)acetonitrile (48 mg, 91%).

LCMS (ESI, m/z): [M+1]⁺=573; RT=0.708 min.

### Step 4: Synthesis of Compound 13

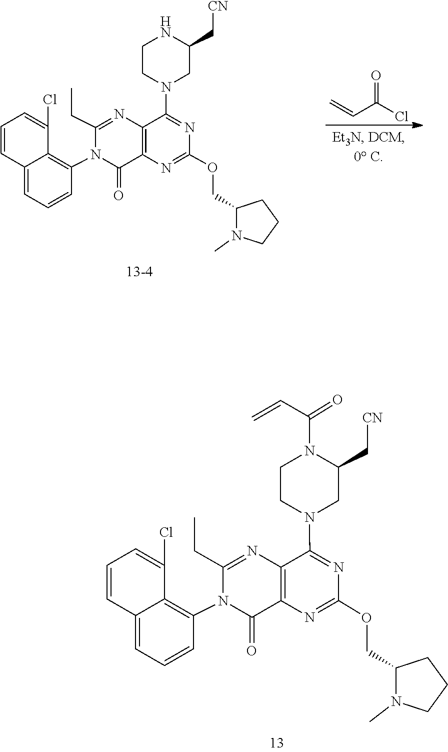

To a cooled (0° C.) solution of 2-((S)-4-(7-(8-chloronaphthalen-1-yl)-6-ethyl-2-(((S)-1-methylpyrrolidin-2-yl)methoxy)-8-oxo-7,8-dihydropyrimido[5,4-d]pyrimidin-4-yl)piperazin-2-yl)acetonitrile (48 mg, 0.084 mmol, 1.0 eq.) and Et₃N (42 mg, 0.419 mmol, 5.0 eq.) in DCM (3.0 mL) was added dropwise a solution of acryloyl chloride (9.1 mg, 0.100 mmol, 1.2 eq.) in DCM (0.5 mL). After addition, the mixture was stirred at 0° C. for 30 min. LCMS showed most of starting material was consumed and desired product formed. Water (10 mL) was added and the organic layer was separated. The aqueous layer was extracted with DCM (5 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(8-chloronaphthalen-1-yl)-6-ethyl-2-(((S)-1-methylpyrrolidin-2-yl)methoxy)-8-oxo-7,8-dihydropyrimido[5,4-d]pyrimidin-4-yl)piperazin-2-yl)acetonitrile (HCOOH salt, 16.0 mg, 30%, 13·0.3HCOOH) (C₃₃H₃₅C₁N₈O₃·0.3HCOOH).

LCMS (ESI, m/z): [M+1]⁺=627; RT=1.050 min.

¹H NMR (400 MHz, DMSO-d₆) δ 8.31 (s, 0.3H), 8.26 (d, J=7.2 Hz, 1H), 8.15 (d, J=8.4 Hz, 1H), 7.83-7.75 (m, 1H), 7.75-7.64 (m, 2H), 7.59 (t, J=7.8 Hz, 1H), 6.97-6.84 (m, 1H), 6.21 (d, J=18.4 Hz, 1H), 5.78 (d, J=11.2 Hz, 1H), 5.38-4.84 (m, 3H), 4.51-4.09 (m, 3H), 3.81-3.50 (m, 2H), 3.26-3.11 (m, 2H), 2.97-2.93 (m, 1H), 2.60-2.51 (m, 2H), 2.35 (s, 3H), 2.22-2.06 (m, 2H), 1.99-1.89 (m 1H), 1.74-1.54 (m, 3H), 1.20-1.03 (m, 3H).

### Example 14

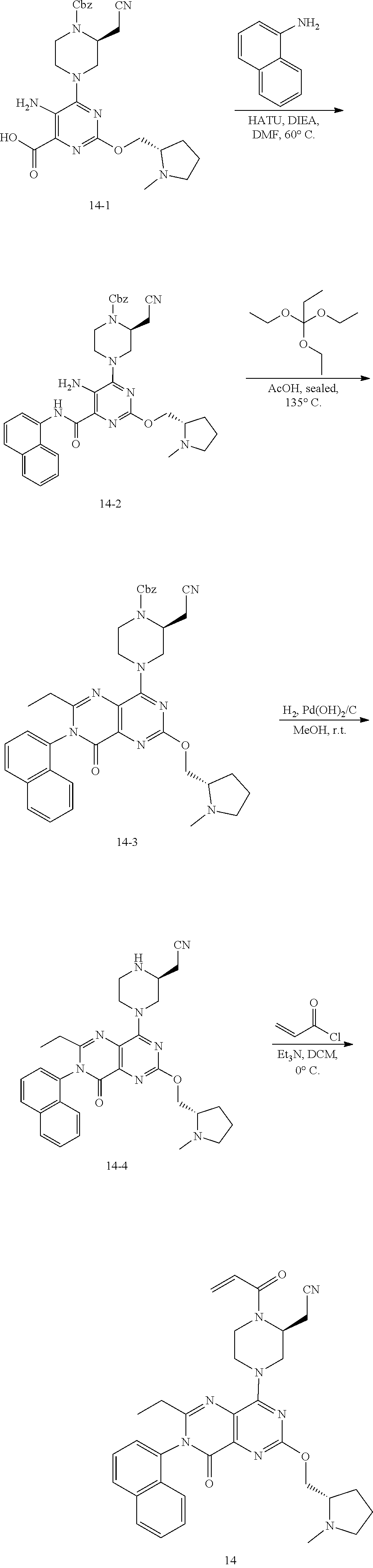

### Step 1: Synthesis of Compound 14-2

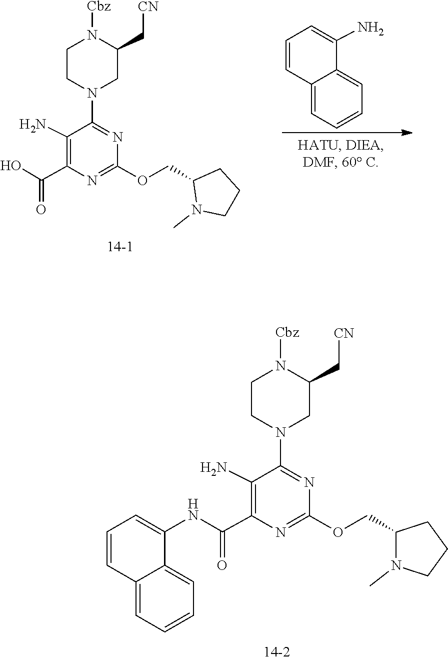 [SMILES: CN1CCC[C@H]1COc1nc(C(=O)O)c(N)c(N2CCN(C(=O)OCc3ccccc3)[C@@H](CC#N)C2)n1 ; Nc1cccc2ccccc12 ; CN1CCC[C@H]1COc1nc(C(=O)Nc2cccc3ccccc23)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1l-yl)-2-(((S)-1-methylpyrrolidin-2-yl)methoxy)pyrimidine-4-carboxylic acid (500 mg, 0.982 mmol, 1.0 eq.) and naphthalen--amine (98.3 mg, 0.688 mmol, 0.7 eq.) in anhydrous DMF (10.0 mL) was added DIEA (0.49 mL, 2.58 mmol, 3.0 eq.), followed by the addition of HATU (373 mg, 0.982 mmol, 1.0 eq.). The reaction mixture was stirred at 60° C. under Ar for 1 h. LCMS showed starting material was consumed and desired product formed. The reaction mixture was cooled to room temperature, diluted with water (40 mL) and extracted with EtOAc (20 mL×3). The combined organic fractions were washed with brine (30 mL), dried over anhydrous Na₂SO₄ and concentrated. The residue was purified by silica column chromatography eluting with DCM/MeOH (10.1, v/v) to obtain benzyl (S′)-4-(5-amino-2-(((S)-1-methylpyrrolidin-2-yl)methoxy)-6-(naphthalen-1-ylcarbamoyl)pyrimidin-4-yl)-2-(cyanomethyl)piperazine-1-carboxylate (424 mg, 68.2%).

LCMS (ESI, m/z): [M+1]⁺=635; RT=1.189 min.

### Step 2: Synthesis of Compound 14-3

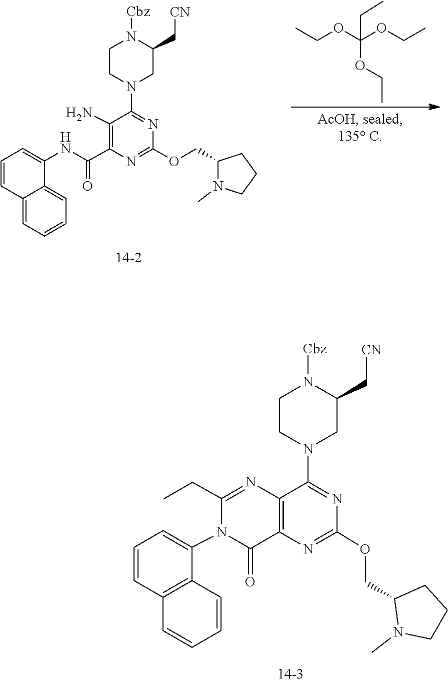 [SMILES: CN1CCC[C@H]1COc1nc(C(=O)Nc2cccc3ccccc23)c(N)c(N2CCN(C(=O)OCc3ccccc3)[C@@H](CC#N)C2)n1 ; CCOC(CC)(OCC)OCC ; CCc1nc2c(N3CCN(C(=O)OCc4ccccc4)[C@@H](CC#N)C3)nc(OC[C@@H]3CCCN3C)nc2c(=O)n1-c1cccc2ccccc12]

To a mixture of benzyl (S)-4-(5-amino-2-(((S)-1-methylpyrrolidin-2-yl)methoxy)-6-(naphthalen-1-ylcarbamoyl)pyrimidin-4-yl)-2-(cyanomethyl)piperazine-1-carboxylate (140 mg, 0.220 mmol, 1.0 eq.) and AcOH (1.4 mL) was added 1,1,1-triethoxypropane (582.9 mg, 3.312 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 25 mL) to adjust pH=7-8, which was extracted with DCM (12 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(6-ethyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (93 mg, 62.9%).

LCMS (ESI, m/z): [M+1]⁺=673; RT=1.271 min.

### Step 3: Synthesis of Compound 14-4

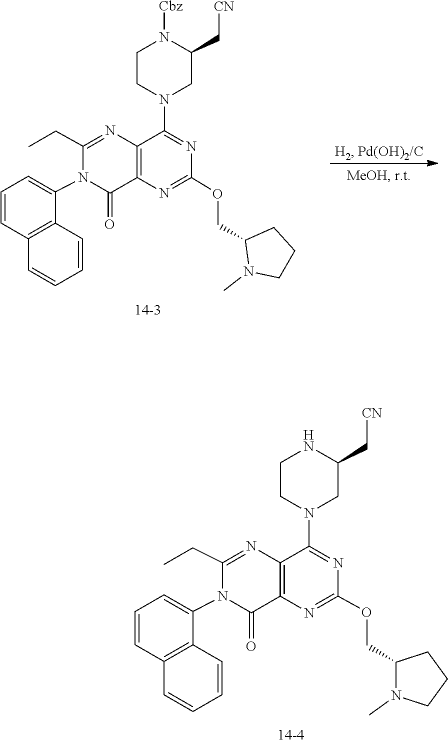 [SMILES: CCc1nc2c(N3CCN(C(=O)OCc4ccccc4)[C@@H](CC#N)C3)nc(OC[C@@H]3CCCN3C)nc2c(=O)n1-c1cccc2ccccc12 ; "H2, Pd(OH)2/C" ; CCc1nc2c(N3CCN[C@@H](CC#N)C3)nc(OC[C@@H]3CCCN3C)nc2c(=O)n1-c1cccc2ccccc12]

To a solution of benzyl (S)-2-(cyanomethyl)-4-(6-ethyl-2-(((S)-1-methylpyrrolidin-2-yl)methoxy)-7-(naphthalen-1-yl)-8-oxo-7,8-dihydropyrimido[5,4-d]pyrimidin-4-yl)piperazine-1-carboxylate (93 mg, 0.138 mmol, 1.0 eq.) in MeOH (8.0 mL) was added Pd(OH)₂/C (20% on carbon, wetted with ca.50% water, 9.8 mg, 0.014 mmol, 0.1 eq.), and the mixture was stirred at room temperature for 1 h under H₂. LCMS showed starting material was consumed and desired product formed. The reaction mixture was filtered and the filtrate was concentrated to dryness to obtain 2-((S)-4-(6-ethyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (36 mg, 48.6%), which was used directly for the next step.

LCMS (ESI, m/z): [M+1]⁺=539; RT=0.355 min;

### 1. Step 4: Synthesis of Compound 14

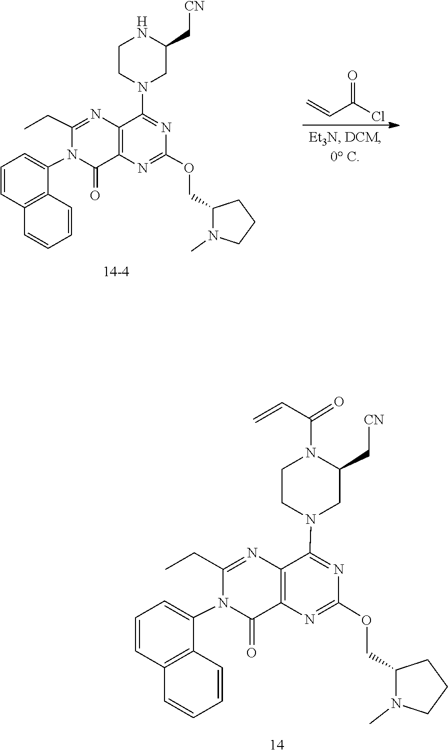

To a cooled (0° C.) solution of 2-((S)-4-(6-ethyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (36 mg, 0.067 mmol, 1.0 eq.) and Et₃N (25 mg, 0.20 mmol, 3.0 eq.) in DCM (5 mL) was added dropwise a solution of acryloyl chloride (7.8 mg, 0.86 mmol, 1.3 eq.) in DCM (0.5 mL). After addition, the mixture was stirred at 0° C. for 30 min. LCMS showed starting material was consumed and desired product formed. Water (20 mL) was added and the organic layer was separated. The aqueous layer was extracted with DCM (10 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(6-ethyl-2-(((S)-1-methylpyrrolidin-2-yl)methoxy)-7-(naphthalen-1-yl)-8-oxo-7,8-dihydropyrimido[5,4-d]pyrimidin-4-yl)piperazin-2-yl)acetonitrile (HCOOH salt, 6.6 mg, 16.7%, 14·HCOOH) (C₃₃H₃₆N₈O₃·HCOOH).

LCMS (ESI, m/z): [M+1]⁺=593; RT=1.008 min;

¹H NMR (400 MHz, DMSO-d₆) δ 8.42 (s, 1H), 8.13 (dd, J=14.0, 8.0 Hz, 2H), 7.75-7.47 (m, 5H), 6.89 (s, 1H), 6.21 (d, J=17.2 Hz, 1H), 5.82-5.71 (m, 1H), 5.62-4.74 (m, 3H), 4.55-4.11 (m, 3H), 3.03-2.77 (m, 4H), 2.67-2.53 (m, 2H), 2.43-2.32 (m, 5H), 2.17 (dd, J=16.8, 8.8 Hz, 1H), 2.04-1.90 (m, 2H), 1.72-1.56 (m, 3H), 1.06 (t, J=7.2 Hz, 3H).

### Example 15

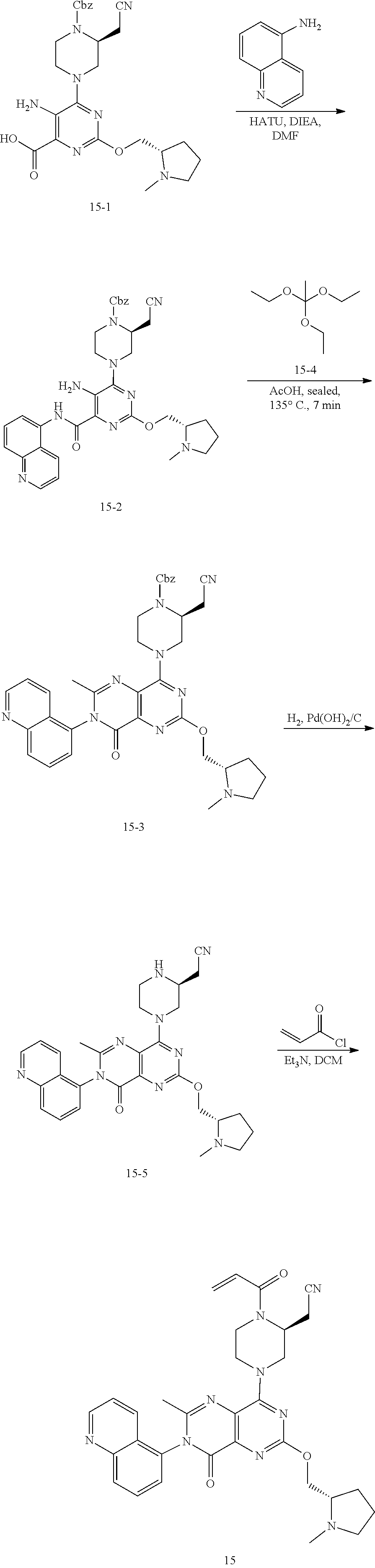

### Step 1: Synthesis of Compound 15-2

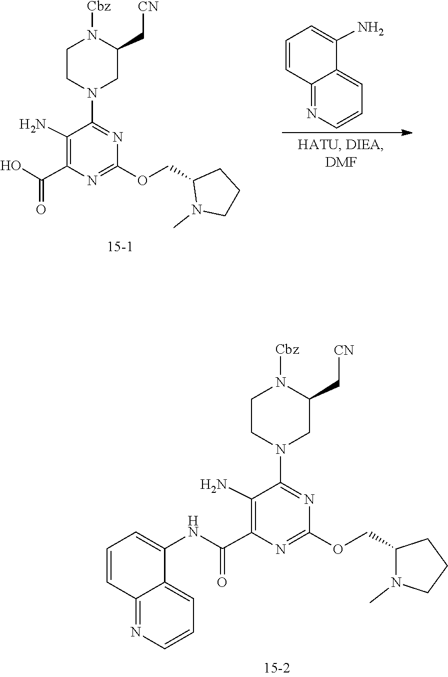 [SMILES: CN1CCC[C@H]1COc1nc(C(=O)O)c(N)c(N2CCN(C(=O)OCc3ccccc3)[C@@H](CC#N)C2)n1 ; Nc1cccc2ncccc12 ; CN1CCC[C@H]1COc1nc(C(=O)Nc2cccc3ncccc23)c(N)c(N2CCN(C(=O)OCc3ccccc3)[C@@H](CC#N)C2)n1]

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (250 mg, 0.491 mmol, 1.0 eq.) and quinolin-5-amine (71 mg, 0.491 mmol, 1.0 eq.) in anhydrous DMF (5.0 mL) was added DIEA (190 mg, 1.473 mmol, 3.0 eq.), followed by the addition of HATU (187 mg, 0.491 mmol, 1.0 eq.). The reaction mixture was stirred at 60° C. under Ar for 1 h. LCMS showed starting material was consumed and desired product formed. The reaction mixture was cooled to room temperature, diluted with EtOAc (30 mL) and washed with brine (3×30 mL), dried over anhydrous Na₂SO₄, filtered and concentrated under reduced pressure. The residue was purified by silica column chromatography eluting with DCM/MeOH (1/0-15:1, v/v) to obtain benzyl (S)-4-(5-amino-2-(((S)-1-methylpyrrolidin-2-yl)methoxy)-6-(quinolin-5-ylcarbamoyl)pyrimidin-4-yl)-2-(cyanomethyl)piperazine-1-carboxylate (140 mg, 45%).

LCMS: Rt: 0.851 min; MS m/z (ESI): 636.3 [M+H]⁺.

### Step 2: Synthesis of Compound 15-3

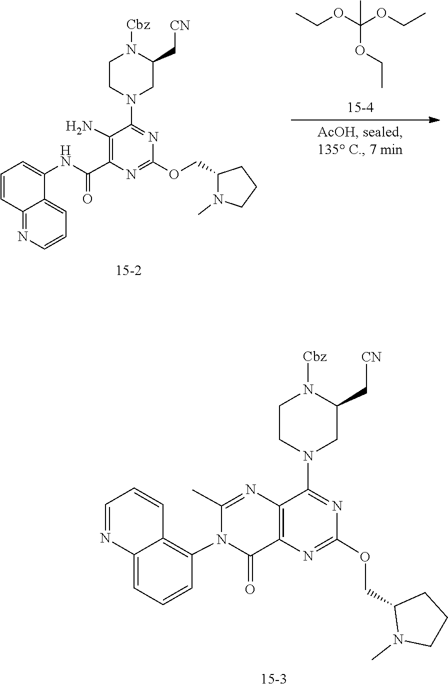 [SMILES: CN1CCC[C@H]1COc1nc(C(=O)Nc2cccc3ncccc23)c(N)c(N2CCN(C(=O)OCc3ccccc3)[C@@H](CC#N)C2)n1 ; CCOC(C)(OCC)OCC ; Cc1nc2c(N3CCN(C(=O)OCc4ccccc4)[C@@H](CC#N)C3)nc(OC[C@@H]3CCCN3C)nc2c(=O)n1-c1cccc2ncccc12]

To a mixture of benzyl (S)-4-(5-amino-2-(((S)-1-methylpyrrolidin-2-yl)methoxy)-6-(quinolin-5-ylcarbamoyl)pyrimidin-4-yl)-2-(cyanomethyl)piperazine-1-carboxylate (130 mg, 0.2046 mmol, 1.0 eq.) and AcOH (0.8 mL) was added 1,1,1-triethoxyethane (506 mg, 3.0694 mmol, 15.0 eq.). The mixture was stirred at 135° C. in a sealed tube for 2.5 min. LCMS showed starting material was consumed and desired product formed. The reaction mixture was adjusted to pH=8-9 with aq. NaHCO₃ solution and extracted with DCM (3×20 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-2-(cyanomethyl)-4-(6-methyl-2-(((S)-1-methylpyrrolidin-2-yl)methoxy)-8-oxo-7-(quinolin-5-yl)-7,8-dihydropyrimido[5,4-d]pyrimidin-4-yl)piperazine-1-carboxylate (110 mg, 82%).

LCMS: Rt: 0.879 min; MS m/z (ESI): 660.3 [M+H]⁺.

### Step 3: Synthesis of Compound 15-5

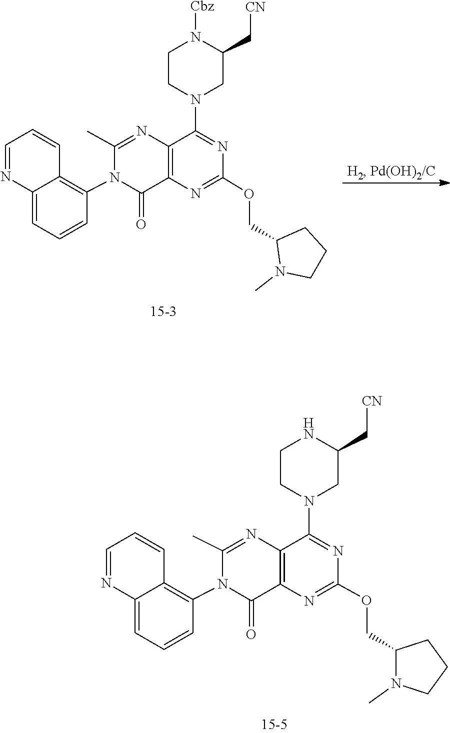 [SMILES: Cc1nc2c(N3CCN(C(=O)OCc4ccccc4)[C@@H](CC#N)C3)nc(OC[C@@H]3CCCN3C)nc2c(=O)n1-c1cccc2ncccc12 ; "H2, Pd(OH)2/C" ; Cc1nc2c(N3CCN[C@@H](CC#N)C3)nc(OC[C@@H]3CCCN3C)nc2c(=O)n1-c1cccc2ncccc12]

To a mixture of benzyl (S)-2-(cyanomethyl)-4-(6-methyl-2-(((S)-1-methylpyrrolidin-2-yl)methoxy)-8-oxo-7-(quinolin-5-yl)-7,8-dihydropyrimido[5,4-d]pyrimidin-4-yl)piperazine-1-carboxylate (110 mg, 0.167 mmol) in MeOH (5.0 mL) was Pd(OH)₂/C (50 mg, 20% wt), and the mixture was stirred at room temperature for 2 h under H₂ (50 psi). LCMS showed starting material was consumed and desired product formed. The resulting mixture was filtered through celite. The filter cake was washed with MeOH (50 mL). The filtrate was concentrated under reduced pressure to obtain 2-((S)-4-(6-methyl-2-(((S)-1-methylpyrrolidin-2-yl)methoxy)-8-oxo-7-(quinolin-5-yl)-7,8-dihydropyrimido[5,4-d]pyrimidin-4-yl)piperazin-2-yl)acetonitrile (84 mg, 95%), which was used directly for the next step without further purification.

LCMS: Rt: 0.352 min; MS m/z (ESI): 526.3 [M+H]⁺.

### Step 4: Synthesis of Compound 15

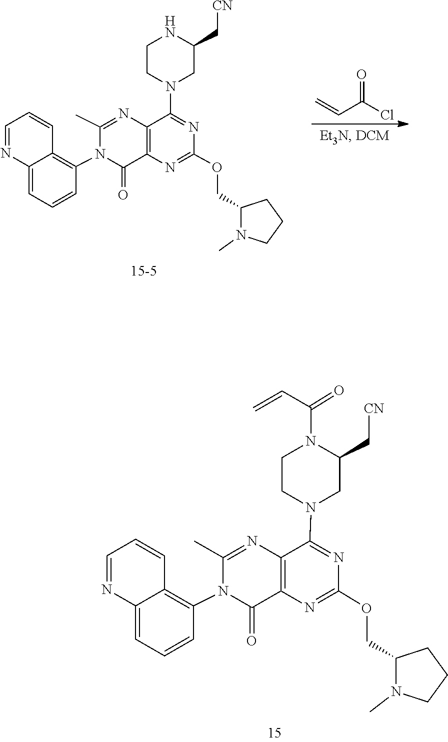

To a mixture of 2-((S)-4-(6-methyl-2-(((S)-1-methylpyrrolidin-2-yl)methoxy)-8-oxo-7-(quinolin-5-yl)-7,8-dihydropyrimido[5,4-d]pyrimidin-4-yl)piperazin-2-yl)acetonitrile (84 mg, 0.16 mmol, 1.0 eq.) and Et₃N (48 mg, 0.48 mmol, 3.0 eq.) in DCM (2 mL) was added dropwise a solution of acryloyl chloride (14.4 mg, 0.16 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by basic prep-HPLC separation to obtain 2-((S)-1-acryloyl-4-(6-methyl-2-(((S)-1-methylpyrrolidin-2-yl)methoxy)-8-oxo-7-(quinolin-5-yl)-7,8-dihydropyrimido[5,4-d]pyrimidin-4-yl)piperazin-2-yl)acetonitrile (20 mg, 21%, 15).

LCMS: Rt: 0.996 min; MS m/z (ESI): 580.2 [M+H]⁺;

¹H NMR (400 MHz, DMSO) δ 9.02 (dd, J=4.1, 1.4 Hz, 1H), 8.24 (d, J=8.5 Hz, 1H), 8.16-8.04 (m, 1H), 7.97 (t, J=8.0 Hz, 1H), 7.84-7.75 (m, 1H), 7.63-7.54 (m, 1H), 6.96-6.80 (m, 1H), 6.21 (dd, J=16.7, 1.8 Hz, 1H), 5.79 (d, J=10.6 Hz, 1H), 5.64-4.70 (m, 3H), 4.58-4.08 (m, 3H), 3.84-3.35 (m, 2H), 3.24-2.88 (m, 4H), 2.61-2.54 (m, 1H), 2.35 (d, J=1.5 Hz, 3H), 2.17 (q, J=8.7 Hz, 1H), 2.03 (d, J=10.7 Hz, 3H), 1.98-1.85 (m, 1H), 1.76-1.48 (m, 3H).

### Example 16

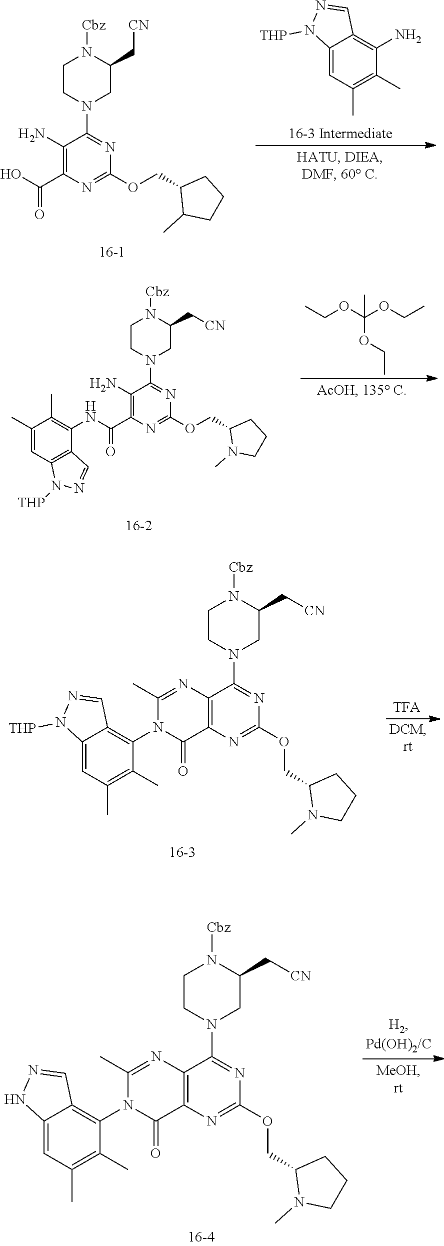

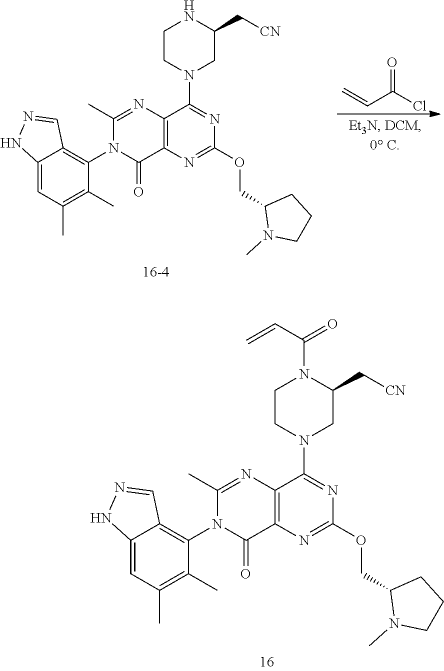

### Step 1: Synthesis of 16-2-Intermediate

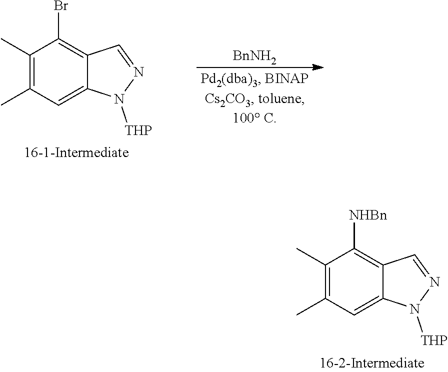

To a solution of N-benzyl-5,6-dimethyl-1-(tetrahydro-2H-pyran-2-yl)-1H-indazol-4-amine (2.00 g, 6.50 mmol, 1.0 eq.) in toluene (20 mL) was added phenylmethanamine (2.08 g, 19.5 mmol, 3.0 eq.), cesium carbonate (6.35 g, 19.5 mmol, 3.0 eq), 1.1′-Binaphthyl-2.2′-diphemyl phosphine (404 mg, 0.65 mmol, 0.1 eq) and Pd₂(dba)₃ (594 mg, 0.65 mmol, 0.1 eq). The reaction mixture was stirred at 100° C. overnight. LCMS analysis showed starting material was consumed and desired product formed. The solution was concentrated and purified by silica gel column chromatography eluting with EtOAc/Pet.ether (10%, v/v) to obtain the title compound (1.9 g, yield 87%).

LCMS (ESI, m/z): [M+1]⁺=336; RT=2.047 min.

### Step 2: Synthesis of 16-2-Intermediate

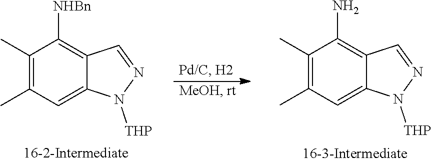

To a solution of N-benzyl-5,6-dimethyl-1-(tetrahydro-2H-pyran-2-yl)-1H-indazol-4-amine (1.9 g) in methanol (20 mL) was added Pd/C (20%, w/w) and stirred at room temperature under hydrogen atmosphere for 1 h. LCMS analysis showed starting material was consumed and desired product formed. The reaction mixture was filtrated and concentrated to obtain the title compound (1.2 g, yield 86%), which was used directly for the next step.

LCMS: [M+1]⁺=246; RT=1.493 min.

### Step 3: Synthesis of Compound 16-2

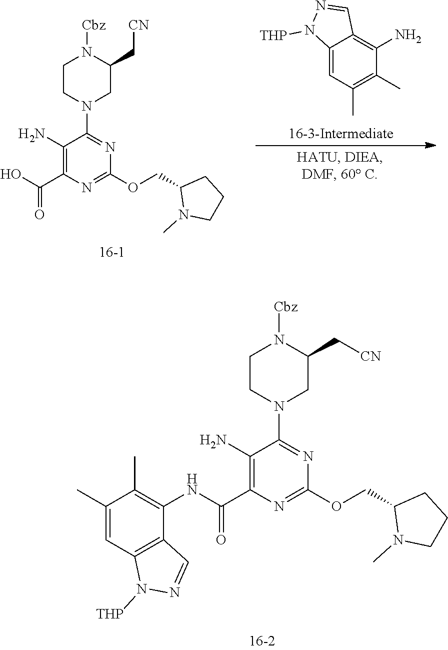

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (400 mg, 0.786 mmol, 1.0 eq.) and 5,6-dimethyl-1-(tetrahydro-2H-pyran-2-yl)-1H-indazol-4-amine (135 mg, 0.550 mmol, 0.7 eq.) in anhydrous DMF (4 mL) was added DIEA (304 mg, 2.358 mmol, 3.0 eq.), followed by the addition of HATU (298 mg, 0.0.786 mmol, 1.0 eq.). The reaction mixture was stirred at 60° C. under Ar for 1 h. LCMS analysis showed starting material was consumed and desired product formed. The reaction mixture was cooled to room temperature, diluted with water (30 mL) and extracted with EtOAc (15 mL×3). The combined organics were washed with brine (20 mL), dried over anhydrous Na₂SO₄ and concentrated. The residue was purified by silica column chromatography eluting with MeOH/DCM (10%, v/v) to obtain benzyl (2S)-4-(5-amino-6-((5,6-dimethyl-1-(tetrahydro-2H-pyran-2-yl)-1H-indazol-4-yl)carbamoyl)-2-(((S)-1-methylpyrrolidin-2-yl)methoxy)pyrimidin-4-yl)-2-(cyanomethyl)piperazine-1-carboxylate (291 mg, 50%).

LCMS (ESI, m/z): [M+1]⁺=637; RT=0.915 min.

### Step 4: Synthesis of Compound 16-3

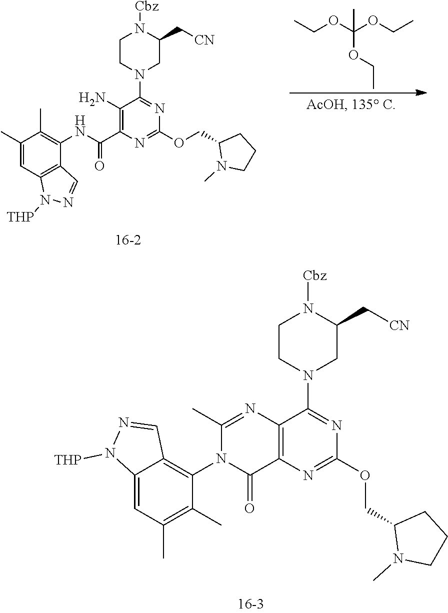

To a mixture of (2S)-4-(5-amino-6-((5,6-dimethyl-1-(tetrahydro-2H-pyran-2-yl)-1H-indazol-4-yl)carbamoyl)-2-(((S)-1-methylpyrrolidin-2-yl)methoxy)pyrimidin-4-yl)-2-(cyanomethyl)piperazine-1-carboxylate (291 mg, 0.396 mmol, 1.0 eq.) and AcOH (1 mL) was added 1,1,1-triethoxyethane (962 mg, 5.940 mmol, 15.0 eq.). The mixture was stirred at 135° C. in a sealed tube for 4 min. LCMS analysis showed starting material was consumed and desired product formed. The reaction mixture was quenched with aq. NaHCO₃ (sat. 40 mL) to adjust pH=7-8, which was extracted with EtOAc (20 mL×3). The combined organics were dried over anhydrous Na₂SO₄ and concentrated to obtain benzyl (2S)-2-(cyanomethyl)-4-(7-(5,6-dimethyl-1-(tetrahydro-2H-pyran-2-yl)-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (331 mg, 110%), which was used directly for the next step.

LCMS (ESI, m/z): [M+1]⁺=761; RT=0.915 min.

### Step 5: Synthesis of Compound 16-4

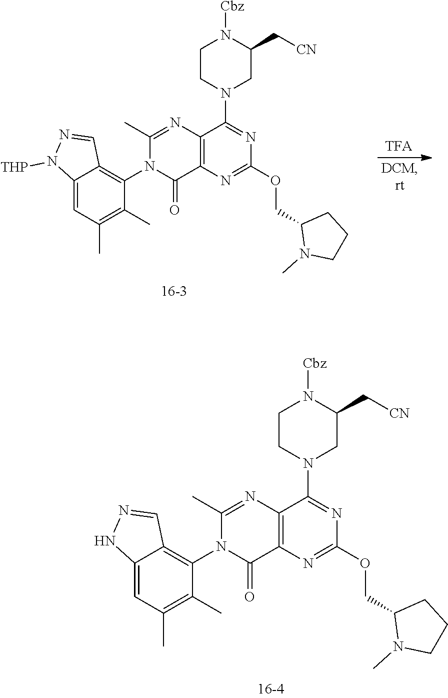

To a solution of benzyl (2S)-2-(cyanomethyl)-4-(7-(5,6-dimethyl-1-(tetrahydro-2H-pyran-2-yl)-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (331 mg, 0.436 mmol, 1.0 eq.) in DCM (3 mL) was added TFA (1 mL), and the mixture was stirred at room temperature for 2 h. LCMS analysis showed starting material was consumed and desired product formed. The reaction mixture was quenched with aq. NaHCO₃ (sat. 10 mL) to adjust pH=7-8, which was extracted with DCM (20 mL×3). The combined organics were dried over anhydrous Na₂SO₄ and concentrated. The residue was purified by prep-TLC eluting with MeOH/DCM (10%, v/v) to obtain benzyl (S)-2-(cyanomethyl)-4-(7-(5,6-dimethyl-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (127 mg, 43%).

LCMS (ESI, m/z): [M+1]⁺=677; RT=0.898 min.

### Step 6: Synthesis of Compound 16-5

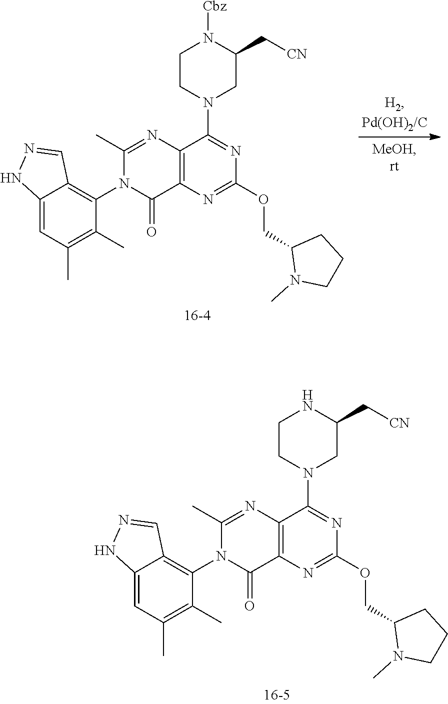 [SMILES: Cc1cc2[nH]ncc2c(-n2c(C)nc3c(N4CCN(C(=O)OCc5ccccc5)[C@@H](CC#N)C4)nc(OC[C@@H]4CCCN4C)nc3c2=O)c1C ; Cc1cc2[nH]ncc2c(-n2c(C)nc3c(N4CCN[C@@H](CC#N)C4)nc(OC[C@@H]4CCCN4C)nc3c2=O)c1C]

To a solution of benzyl (S)-2-(cyanomethyl)-4-(7-(5,6-dimethyl-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazine-1-carboxylate (127 mg, 0.189 mmol, 1.0 eq.) in MeOH (3 mL) was added Pd(OH)₂/C (10% on carbon, wetted with ca.50% water, 12 mg), and the reaction mixture was stirred at room temperature under H₂ for 2 h. LCMS analysis showed starting material was consumed and desired product formed. The resulting mixture was filtered through celite. The filtrate was concentrated to dryness to obtain 2-((S)-4-(7-(5,6-dimethyl-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (92 mg, 90%).

LCMS (ESI, m/z): [M+1]⁺=543; RT=0.332 min.

### Step 7: Synthesis of Compound 16

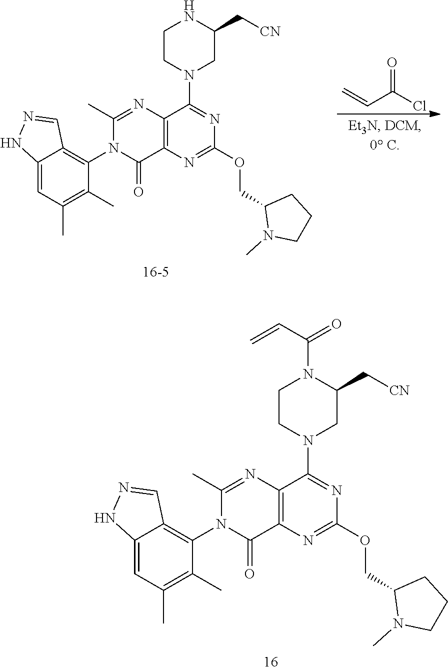

To a cooled (0° C.) solution of 2-((S)-4-(7-(5,6-dimethyl-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (92 mg, 0.170 mmol, 1.0 eq.) and Et₃N (51 mg, 0.510 mmol, 3.0 eq.) in DCM (5 mL) was added dropwise a solution of acryloyl chloride (12 mg, 0.136 mmol, 0.8 eq.) in DCM (0.5 mL). After addition, the mixture was stirred at 0° C. for 30 min. LCMS analysis showed starting material was consumed and desired product formed. Water (3 mL) was added and the organic layer was separated. The aqueous layer was extracted with DCM (5 mL×3). The combined organic fractions were dried over anhydrous Na₂SO₄ and concentrated. The residue was purified by prep-HPLC (ACN-H₂O+0.1% NH₄HCO₃) to obtain 2-((S)-1-acryloyl-4-(7-(5,6-dimethyl-1H-indazol-4-yl)-6-methyl-2-(((S)-1-methylpyrrolidin-2-yl)methoxy)-8-oxo-7,8-dihydropyrimido[5,4-d]pyrimidin-4-yl)piperazin-2-yl)acetonitrile (5.82 mg, 5.8%, 16) (C₃₁H₃₆N₁₀O₃).

LCMS (ESI, m/z): [M+1]⁺=597; RT=1.061 min;

¹H NMR (400 MHz, CDCl₃) δ 8.47 (s, 1H), 7.63 (d, J=12.8 Hz, 1H), 7.37 (s, 1H), 6.61 (s, 1H), 6.40 (d, J=16.6 Hz, 1H), 5.85-5.80 (m, 1H), 5.09 (s, 2H), 4.87-4.57 (m, 3H), 4.06-3.83 (m, 1H), 3.66-3.34 (m, 4H), 2.85-2.71 (m, 5H), 2.42 (s, 3H), 2.26-2.18 (m, 1H), 2.11 (s, 3H), 2.08 (s, 3H), 2.02-1.93 (m, 2H), 1.77-1.61 (m, 1H), 1.47-1.23 (m, 1H), 0.91 (t, J=7.3 Hz, 1H).

### Example 17

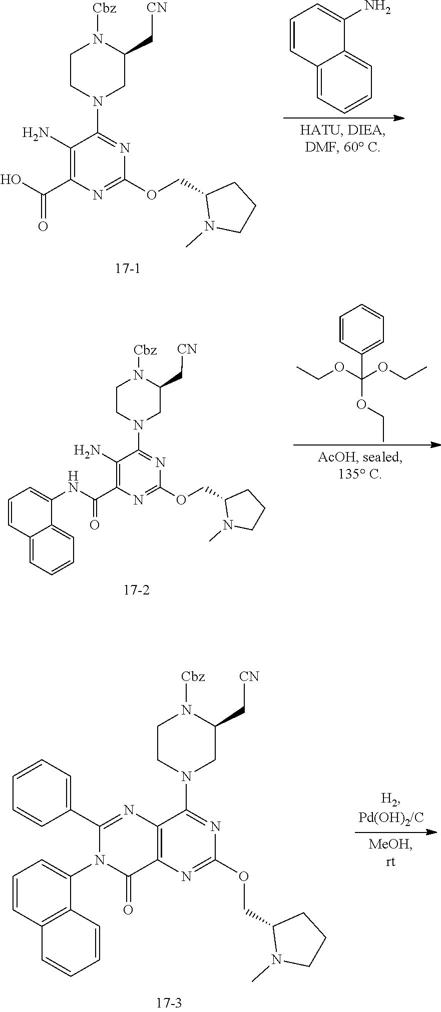 [SMILES: CN1CCC[C@H]1COc1nc(C(=O)O)c(N)c(N2CCN(C(=O)OCc3ccccc3)[C@@H](CC#N)C2)n1 ; Nc1cccc2ccccc12 ; CN1CCC[C@H]1COc1nc(C(=O)Nc2cccc3ccccc23)c(N)c(N2CCN(C(=O)OCc3ccccc3)[C@@H](CC#N)C2)n1 ; CCOC(OCC)(OCC)c1ccccc1 ; CN1CCC[C@H]1COc1nc(N2CCN(C(=O)OCc3ccccc3)[C@@H](CC#N)C2)c2nc(-c3ccccc3)n(-c3cccc4ccccc34)c(=O)c2n1]

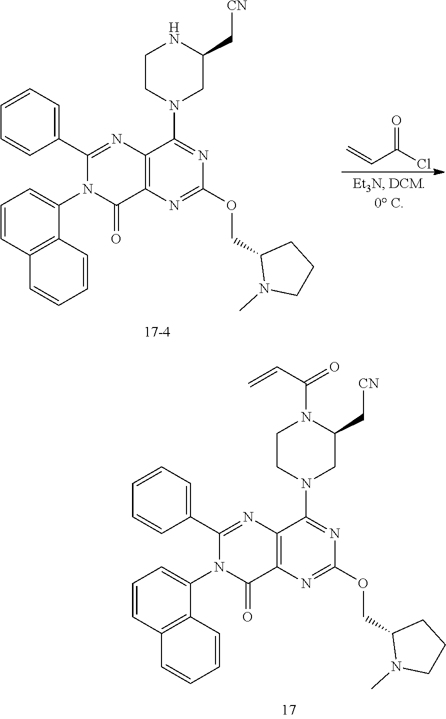

### Step 1: Synthesis of Compound 17-1

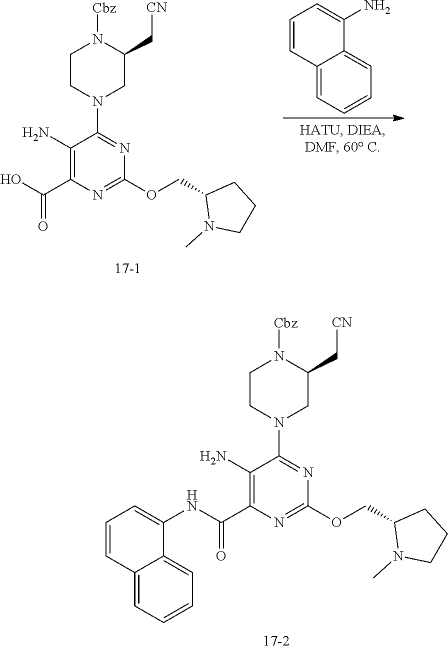 [SMILES: CN1CCC[C@H]1COc1nc(C(=O)O)c(N)c(N2CCN(C(=O)OCc3ccccc3)[C@@H](CC#N)C2)n1 ; Nc1cccc2ccccc12 ; CN1CCC[C@H]1COc1nc(C(=O)Nc2cccc3ccccc23)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (500 mg, 0.982 mmol, 1.0 eq.) and naphthalen-1-amine (98.3 mg, 0.688 mmol, 0.7 eq.) in anhydrous DMF (10.0 mL) was added DIEA (0.49 mL, 2.58 mmol, 3.0 eq.), followed by the addition of HATU (373 mg, 0.982 mmol, 1.0 eq.). The reaction mixture was stirred at 60° C. under Ar for 1 h. LCMS showed starting material was consumed and desired product formed. The reaction mixture was cooled to room temperature, diluted with water (40 mL) and extracted with EtOAc (20 mL×3). The combined organic fractions were washed with brine (30 mL), dried over anhydrous Na₂SO₄ and concentrated. The residue was purified by silica column chromatography eluting with DCM/MeOH (10:1, v/v) to obtain benzyl (S)-4-(5-amino-2-(((S)-1-methylpyrrolidin-2-yl)methoxy)-6-(naphthalen-1-ylcarbamoyl)pyrimidin-4-yl)-2-(cyanomethyl)piperazine-1-carboxylate (424 mg, 68.2%).

LCMS (ESI, m/z): [M+1]⁺=635; RT=1.189 min. 2. Step 2: Synthesis of Compound 17-3

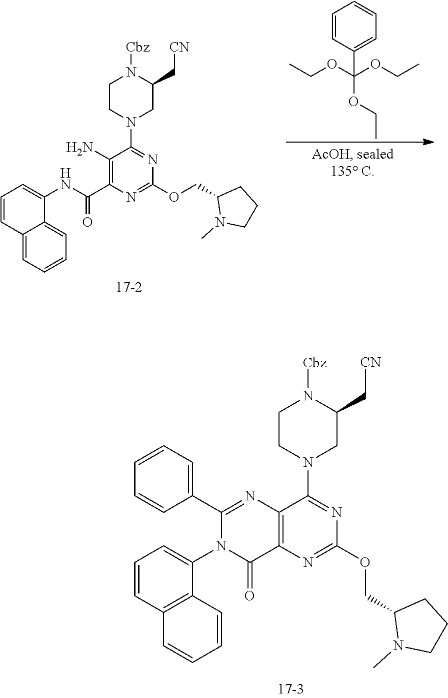 [SMILES: CN1CCC[C@H]1COc1nc(C(=O)Nc2cccc3ccccc23)c(N)c(N2CCN(C(=O)OCc3ccccc3)[C@@H](CC#N)C2)n1 ; CCOC(OCC)(OCC)c1ccccc1 ; CN1CCC[C@H]1COc1nc(N2CCN(C(=O)OCc3ccccc3)[C@@H](CC#N)C2)c2nc(-c3ccccc3)n(-c3cccc4ccccc34)c(=O)c2n1]

To a mixture of benzyl (S)-4-(5-amino-2-(((S)-1-methylpyrrolidin-2-yl)methoxy)-6-(naphthalen-1-ylcarbamoyl)pyrimidin-4-yl)-2-(cyanomethyl)piperazine-1-carboxylate (200 mg, 0.315 mmol, 1.0 eq.) and AcOH (2.0 mL) was added (triethoxymethyl)benzene (1.0 g, 4.725 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 50 mL) to adjust pH=7-8, which was extracted with DCM (30 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(2-(((S)-1-methylpyrrolidin-2-yl)methoxy)-7-(naphthalen-1-yl)-8-oxo-6-phenyl-7,8-dihydropyrimido[5,4-d]pyrimidin-4-yl)piperazine-1-carboxylate (85 mg, 37.5%).

LCMS (ESI, m/z): [M+1]⁺=721; RT=1.265 min.

### Step 3: Synthesis of Compound 17-4

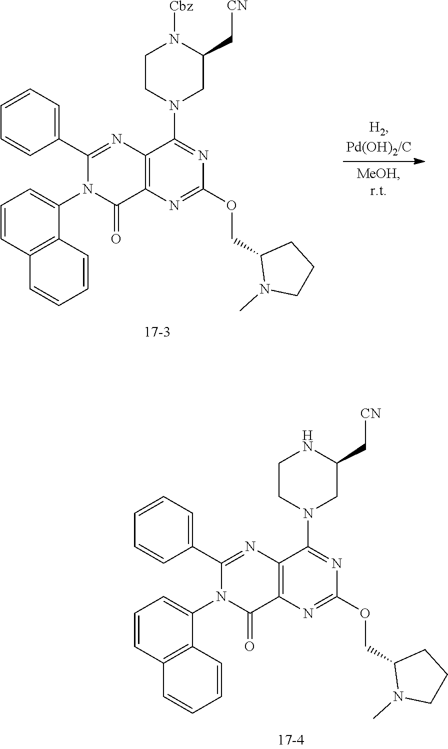 [SMILES: CN1CCC[C@H]1COc1nc(N2CCN(C(=O)OCc3ccccc3)[C@@H](CC#N)C2)c2nc(-c3ccccc3)n(-c3cccc4ccccc34)c(=O)c2n1 ; CN1CCC[C@H]1COc1nc(N2CCN[C@@H](CC#N)C2)c2nc(-c3ccccc3)n(-c3cccc4ccccc34)c(=O)c2n1]

To a solution of benzyl (S)-2-(cyanomethyl)-4-(2-(((S)-1-methylpyrrolidin-2-yl)methoxy)-7-(naphthalen-1-yl)-8-oxo-6-phenyl-7,8-dihydropyrimido[5,4-d]pyrimidin-4-yl)piperazine-1-carboxylate (85 mg, 0.118 mmol, 1.0 eq.) in MeOH (5.0 mL) was added Pd(OH)₂/C (20% on carbon, wetted with ca.50% water, 8.46 mg, 0.012 mmol, 0.1 eq.), and the mixture was stirred at room temperature for 1 h under H₂. LCMS showed starting material was consumed and desired product formed. The reaction mixture was filtered and the filtrate was concentrated to dryness to obtain 2-((S)-4-(2-(((S)-1-methylpyrrolidin-2-yl)methoxy)-7-(naphthalen-1-yl)-8-oxo-6-phenyl-7,8-dihydropyrimido[5,4-d]pyrimidin-4-yl)piperazin-2-yl)acetonitrile (69 mg, 99%), which was used directly for the next step.

LCMS (ESI, m/z): [M+1]⁺=587; RT=0.763 min;

### Step 4: Synthesis of Compound 17

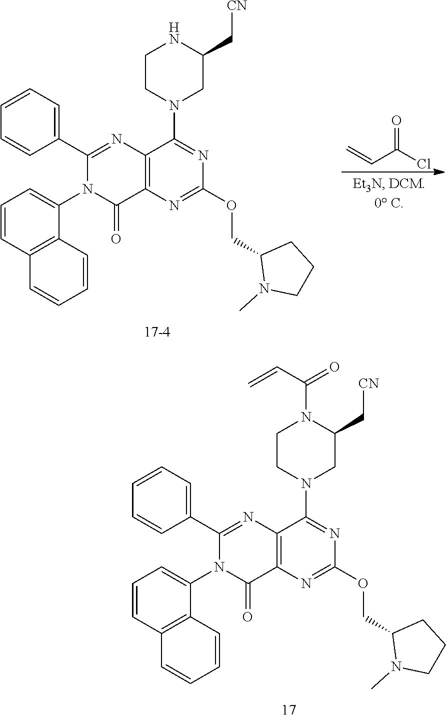

To a cooled (0° C.) solution of 2-((S)-4-(2-(((S)-1-methylpyrrolidin-2-yl)methoxy)-7-(naphthalen-1-yl)-8-oxo-6-phenyl-7,8-dihydropyrimido[5,4-d]pyrimidin-4-yl)piperazin-2-yl)acetonitrile (69 mg, 0.118 mmol, 1.0 eq.) and Et₃N (45.6 mg, 0.354 mmol, 3.0 eq.) in DCM (5 mL) was added dropwise a solution of acryloyl chloride (10.6 mg, 0.118 mmol, 1 eq.) in DCM (5.0 mL). After addition, the mixture was stirred at 0° C. for 30 min. LCMS showed starting material was consumed and desired product formed. Water (20 mL) was added and the organic layer was separated. The aqueous layer was extracted with DCM (10 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(2-(((S)-1-methylpyrrolidin-2-yl)methoxy)-7-(naphthalen-1-yl)-8-oxo-6-phenyl-7,8-dihydropyrimido[5,4-d]pyrimidin-4-yl)piperazin-2-yl)acetonitrile (HCOOH salt, 2.37 mg, 3.2%, 17·HCOOH) (C₃₇H₃₆N₈O₃·HCOOH).

LCMS (ESI, m/z): [M+1]⁺=641; RT=1.061 min;

¹H NMR (400 MHz, DMSO-d₆) δ 8.32 (s, 1H), 8.02-7.87 (m, 2H), 7.79-7.70 (m, 1H), 7.61-7.4 (m, 3H), 7.34-7.21 (m, 2H), 7.19-6.99 (m, 3H), 6.96-6.78 (m, 2H), 6.19 (d, J=16.4 Hz, 1H), 5.76 (d, J=12.0 Hz, 1H), 5.15-4.84 (m, 3H), 4.40-4.33 (m, 1H), 4.19 (dd, J=16.4, 9.0 Hz, 1H), 3.22-3.09 (m, 4H), 2.95 (dd, J=9.2, 3.6 Hz, 2H), 2.61 (d, J=6.0 Hz, 2H), 2.37 (s, 3H), 2.19 (dd, J=16.8, 8.4 Hz, 1H), 1.99-1.92 (m, 1H), 1.74-1.58 (m, 3H).

### Example 18

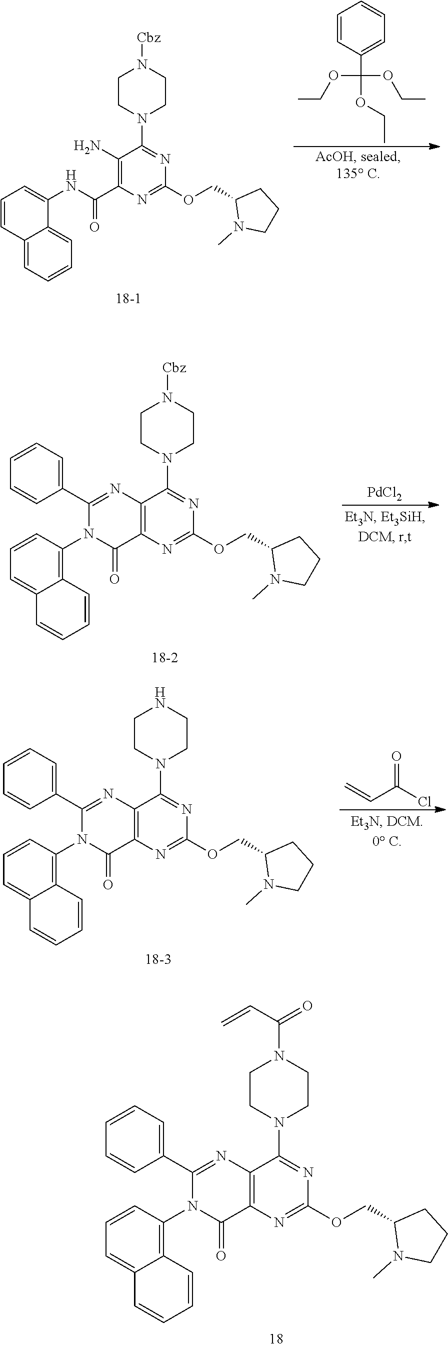

### Step 1: Synthesis of Compound 18-2

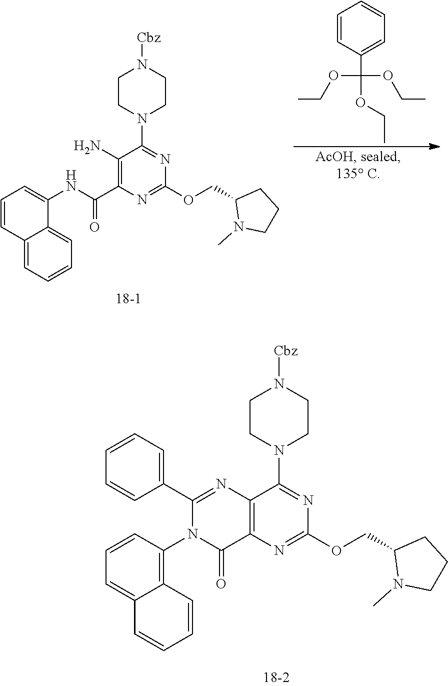 [SMILES: CN1CCC[C@H]1COc1nc(C(=O)Nc2cccc3ccccc23)c(N)c(N2CCN(C(=O)OCc3ccccc3)CC2)n1 ; CCOC(OCC)(OCC)c1ccccc1 ; CN1CCC[C@H]1COc1nc(N2CCN(C(=O)OCc3ccccc3)CC2)c2nc(-c3ccccc3)n(-c3cccc4ccccc34)c(=O)c2n1]

To a mixture of benzyl (S)-4-(5-amino-2-((1-methylpyrrolidin-2-yl)methoxy)-6-(naphthalen-1-ylcarbamoyl)pyrimidin-4-yl)piperazine-1-carboxylate (65.6 mg, 0.11 mmol, 1.0 eq.) and AcOH (0.4 mL) was added (triethoxymethyl)benzene (371 mg, 1.65 mmol, 15.0 eq.). The mixture was stirred at 135° C. in a sealed tube for 2.5 min. LCMS showed starting material was consumed and desired product formed. The reaction mixture was quenched with aq. NaHCO₃ (sat. 25 mL) to adjust pH=7-8, which was extracted with DCM (15 mL×3). The combined organic fractions were dried over anhydrous Na₂SO₄ and concentrated. The residue was purified by prep-TLC eluting with DCM/MeOH (10:1, v/v) to obtain benzyl (S)-4-(2-((1-methylpyrrolidin-2-yl)methoxy)-7-(naphthalen-1-yl)-8-oxo-6-phenyl-7,8-dihydropyrimido[5,4-d]pyrimidin-4-yl)piperazine-1-carboxylate (27.3 mg, 36.4%).

LCMS (ESI, m/z): [M+1]⁺=682; RT=1.277 min.

### Step 2: Synthesis of Compound 18-3

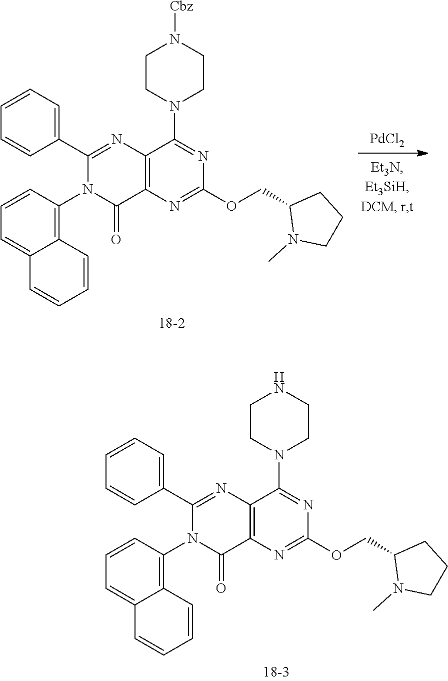

To a solution of benzyl (S)-4-(2-((1-methylpyrrolidin-2-yl)methoxy)-7-(naphthalen-1-yl)-8-oxo-6-phenyl-7,8-dihydropyrimido[5,4-d]pyrimidin-4-yl)piperazine-1-carboxylate (65 mg, 0.095 mmol, 1.0 eq.) in DCM (4.0 mL) was added Et₃N (65 mg, 0.095 mmol, 10.0 eq.) and Et₃SiH (110.5 mg, 0.095 mmol, 1.0 eq.), followed by the addition of PdCl (16. mg, 0.951 mmol, 10.0 eq.). The reaction mixture was stirred at room temperature for 1 h. LCMS showed starting material was consumed and desired product formed. The reaction mixture was concentrated to dryness to obtain (S)-6-((1-methylpyrrolidin-2-yl)methoxy)-3-(naphthalen-1-yl)-2-phenyl-8-(piperazin-1-yl)pyrimido[5,4-d]pyrimidin-4(3H)-one (51 mg, 99%), which was used directly for the next step.

LCMS (ESI, m/z): [M+1]⁺=548; RT=0.868 min.

### Step 3: Synthesis of Compound 18

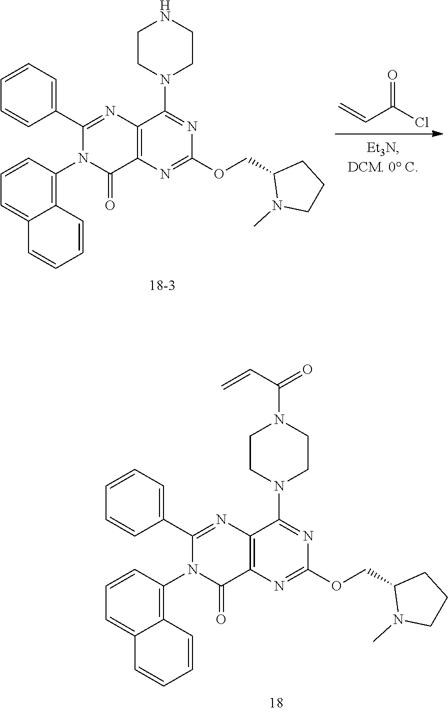

To a cooled (0° C.) solution of (S)-6-((1-methylpyrrolidin-2-yl)methoxy)-3-(naphthalen-1-yl)-2-phenyl-8-(piperazin-1-yl)pyrimido[5,4-d]pyrimidin-4(3H)-one (52.2 mg, 0.095 mmol, 1.0 eq.) and Et₃N (19.3 mg, 0.285 mmol, 0.7 eq.) in DCM (3 mL) was added dropwise a solution of acryloyl chloride (6.02 mg, 0.095 mmol, 1.0 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) to obtain (S)-8-(4-acryloylpiperazin-1-yl)-6-((1-methylpyrrolidin-2-yl)methoxy)-3-(naphthalen-1-yl)-2-phenylpyrimido[5,4-d]pyrimidin-4(3H)-one (HCOOH salt, 3.0 mg, 5.2%, 18·HCOOH) (C₃₅H₃₅N₇O₃·HCOOH).

LCMS (ESI, m/z): [M+1]⁺=602; RT=1.055 min;

¹H NMR (400 MHz, DMSO-d₆) δ 8.32 (s, 1H), 8.06-7.86 (m, 2H), 7.77 (dd, J=6.4, 3.6 Hz, 1H), 7.55 (dd, J=6.4, 2.6 Hz, 3H), 7.46 (t, J=7.6 Hz, 1H), 7.31-7.19 (m, 2H), 7.14 (t, J=7.6 Hz, 1H), 7.05 (t, J=7.6 Hz, 2H), 6.85 (dd, J=16.4, 10.4 Hz, 1H), 6.15 (dd, J=16.6, 2.4 Hz, 1H), 5.71 (dd, J=10.4, 2.4 Hz, 1H), 4.59-4.01 (m, 6H), 3.75 (d, J=22.8 Hz, 5H), 3.01-2.92 (m, 1H), 2.64-2.55 (m, 1H), 2.35 (d, J=15.6 Hz, 3H), 2.29-2.10 (m, 1H), 1.96 (dd, J=11.6, 7.6 Hz, 1H), 1.80-1.54 (m, 3H).

### Example 19

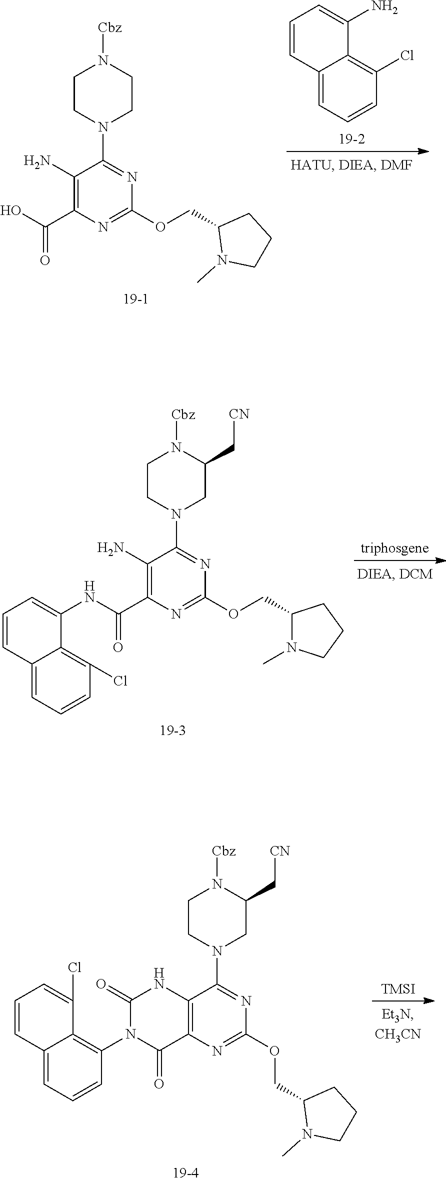

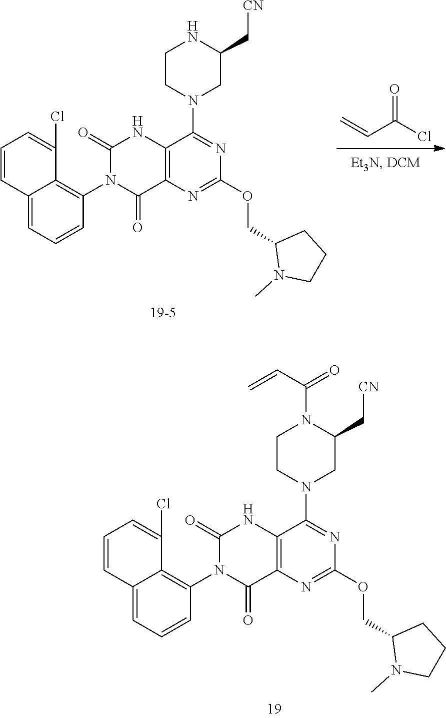

### Step 1: Synthesis of Compound 19-3

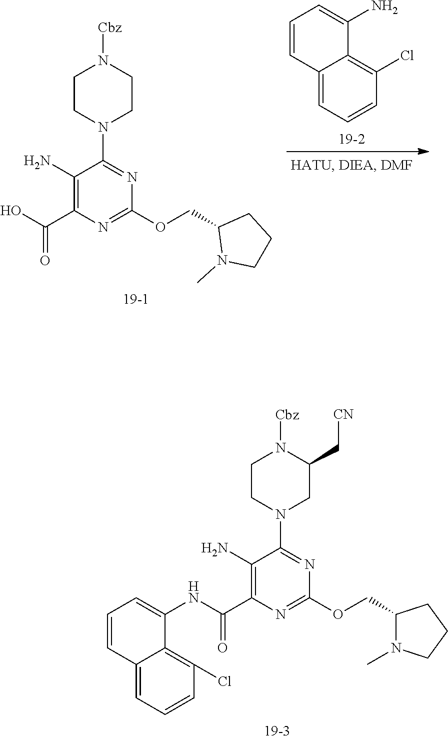 [SMILES: CN1CCC[C@H]1COc1nc(C(=O)O)c(N)c(N2CCN(C(=O)OCc3ccccc3)CC2)n1 ; Nc1cccc2cccc(Cl)c12 ; CN1CCC[C@H]1COc1nc(C(=O)Nc2cccc3cccc(Cl)c23)c(N)c(N2CCN(C(=O)OCc3ccccc3)[C@@H](CC#N)C2)n1]

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (200 mg, 0.393 mmol, 1.0 eq.) and 8-chloronaphthalen-1-amine (49 mg, 0.275 mmol, 1.0 eq.) in anhydrous DMF (5.0 mL) was added DIEA (152 mg, 1.179 mmol, 3.0 eq.), followed by the addition of HATU (149 mg, 0.393 mmol, 1.0 eq.). The reaction mixture was stirred at 60° C. under Ar for 1 h. LCMS showed starting material was consumed and desired product formed. The reaction mixture was cooled to room temperature, diluted with EtOAc (30 mL) and washed with brine (3×30 mL), dried over anhydrous Na₂SO₄, filtered and concentrated under reduced pressure. The residue was purified by silica column chromatography eluting with DCM/MeOH (1/0-10:1, v/v) to obtain benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (200 mg, 51%).

LCMS: Rt: 0.955 min; MS m/z (ESI): 669.3 [M+H]⁺.

### Step 2: Synthesis of Compound 19-4

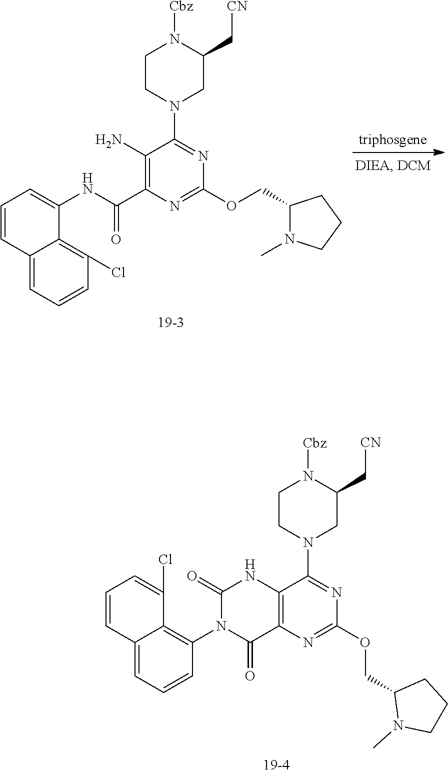 [SMILES: CN1CCC[C@H]1COc1nc(C(=O)Nc2cccc3cccc(Cl)c23)c(N)c(N2CCN(C(=O)OCc3ccccc3)[C@@H](CC#N)C2)n1 ; CN1CCC[C@H]1COc1nc(N2CCN(C(=O)OCc3ccccc3)[C@@H](CC#N)C2)c2[nH]c(=O)n(-c3cccc4cccc(Cl)c34)c(=O)c2n1]

To a mixture of benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (80 mg, 0.120 mmol, 1.0 eq.) in DCM (2 mL) was added DIEA (46 mg, 0.360 mmol), followed by triphosgene (35 mg, 0.120 mmol) at 0° C. The mixture was stirred at 0° C. for 1 h under N₂. TLC (DCM/MeOH=10/1) showed starting material was consumed. The reaction mixture was quenched with water (15 mL) and extracted with DCM (3×15 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-4-(7-(8-chloronaphthalen-1-yl)-2-(((S)-1-methylpyrrolidin-2-yl)methoxy)-6,8-dioxo-5,6,7,8-tetrahydropyrimido[5,4-d]pyrimidin-4-yl)-2-(cyanomethyl)piperazine-1-carboxylate (58 mg, 70%).

LCMS: Rt: 0.875 min; MS m/z (ESI): 695.2 [M+H]⁺.

### Step 3: Synthesis of Compound 19-5

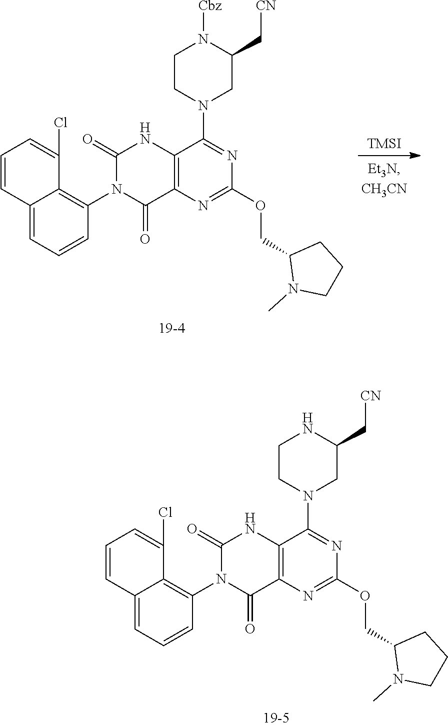 [SMILES: "TMSI" ; CN1CCC[C@H]1COc1nc(N2CCN(C(=O)OCc3ccccc3)[C@@H](CC#N)C2)c2[nH]c(=O)n(-c3cccc4cccc(Cl)c34)c(=O)c2n1 ; CN1CCC[C@H]1COc1nc(N2CCN[C@@H](CC#N)C2)c2[nH]c(=O)n(-c3cccc4cccc(Cl)c34)c(=O)c2n1]

To a mixture of benzyl (S)-4-(7-(8-chloronaphthalen-1-yl)-2-(((S)-1-methylpyrrolidin-2-yl)methoxy)-6,8-dioxo-5,6,7,8-tetrahydropyrimido[5,4-d]pyrimidin-4-yl)-2-(cyanomethyl)piperazine-1-carboxylate (58 mg, 0.0836 mmol, 1 eq.) in CH₃CN (2.0 mL) was added TMSI (134 mg, 0.6686 mmol, 8 eq.), and the mixture was stirred at 35° C. for 1 h under N₂. TLC (DCM/MeOH=10/1) showed the starting material was consumed. The resulting mixture was added with Et₃N (135 mg, 1.3376 mmol, 16 eq.) and stirred at room temperature for 15 min. The mixture was concentrated under reduced pressure. The residue was diluted with H₂O (15 mL) and extracted with DCM/MeOH (10/1, 3×15 mL). The combined organic layers were dried over anhydrous Na₂SO₄, filtered and concentrated under reduced pressure. The residue was purified by prep-TLC eluting with DCM/MeOH (8:1, v/v) to obtain 2-((S)-4-(7-(8-chloronaphthalen-1-yl)-2-(((S)-1-methylpyrrolidin-2-yl)methoxy)-6,8-dioxo-5,6,7,8-tetrahydropyrimido[5,4-d]pyrimidin-4-yl)piperazin-2-yl)acetonitrile (46 mg, 100%).

LCMS: Rt: 0.379 min; MS m/z (ESI): 561.0 [M+H]⁺.

### Step 4: Synthesis of Compound 19

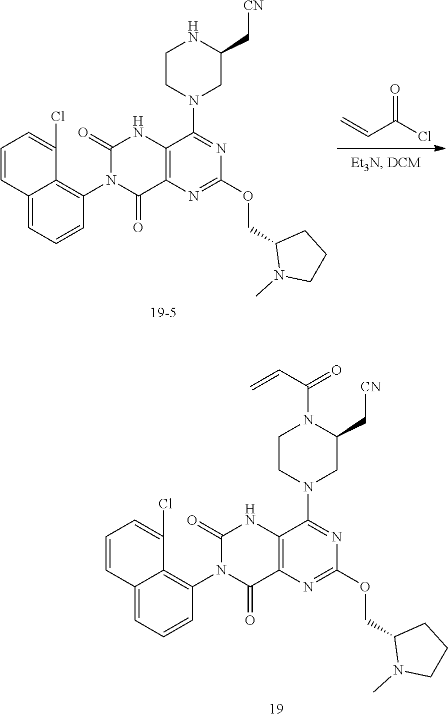

To a mixture of 2-((S)-4-(7-(8-chloronaphthalen-1-yl)-2-(((S)-1-methylpyrrolidin-2-yl)methoxy)-6,8-dioxo-5,6,7,8-tetrahydropyrimido[5,4-d]pyrimidin-4-yl)piperazin-2-yl)acetonitrile (46 mg, 0.0821 mmol, 1.0 eq.) and Et₃N (25 mg, 0.2463 mmol, 3.0 eq.) in DCM (2 mL) and CH₃CN (2 mL) was added dropwise a solution of acryloyl chloride (7.4 mg, 0.0821 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain 2-((S)-1-acryloyl-4-(7-(8-chloronaphthalen-1-yl)-2-(((S)-1-methylpyrrolidin-2-yl)methoxy)-6,8-dioxo-5,6,7,8-tetrahydropyrimido[5,4-d]pyrimidin-4-yl)piperazin-2-yl)acetonitrile (HCOOH salt, 8 mg, 16%, 19-HCOOH).

LCMS: Rt: 0.737 min; MS m/z (ESI): 615.3 [M+H]⁺;

¹H NMR (400 MHz, DMSO) δ 8.27 (s, 1.42H, HCOOH), 8.10-7.97 (m, 2H), 7.70-7.62 (m, 1H), 7.58-7.54 (m, 1H), 7.52-7.45 (m, 1H), 7.41-7.30 (m, 1H), 7.00-6.80 (m, 1H), 6.20 (d, J=16.5 Hz, 1H), 5.77 (d, J=10.4 Hz, 1H), 5.42-4.60 (m, 1H), 4.50-3.91 (m, 4H), 3.71-3.60 (m, 1H), 3.08-2.86 (m, 4H), 2.82-2.61 (m, 2H), 2.43 (d, J=11.8 Hz, 3H), 2.35-2.25 (s, 1H), 2.04-1.87 (m, 1H), 1.81-1.49 (m, 3H).

### Example 20

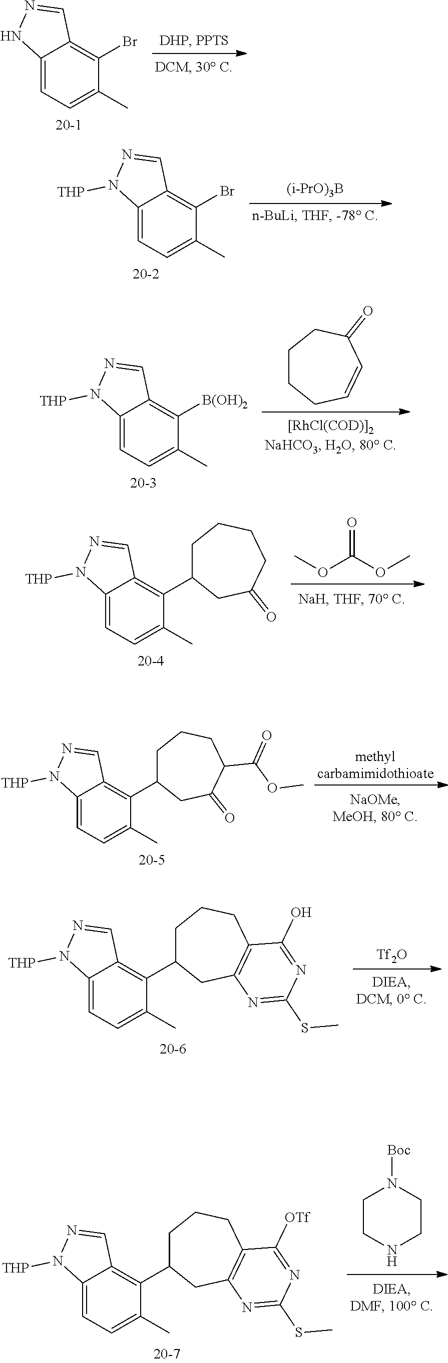

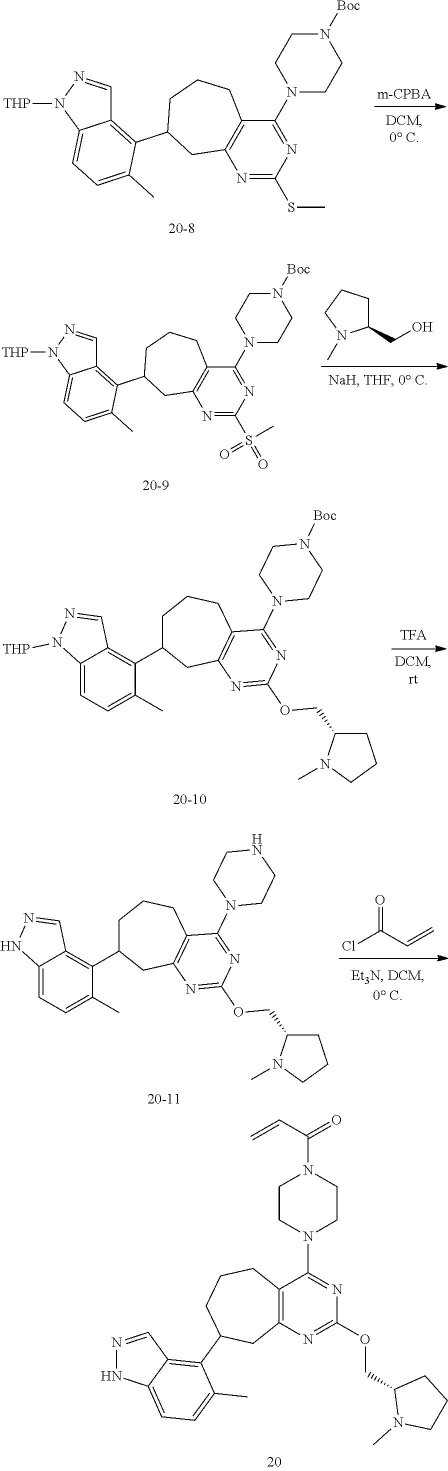

### 3. Step 1: Synthesis of Compound 20-2

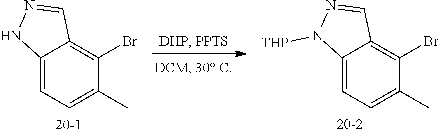

To a solution of 4-bromo-5-methyl-H-indazole (14.0 g, 66.67 mmol, 1.0 eq.) in anhydrous DCM (30 mL) was added PPTS (1.68 g, 6.68 mmol 0.1 eq.) at room temperature. Then DHP (16.83 g, 200.02 mmol, 3 eq.) was added in one portion. The reaction mixture was stirred at 30° C. overnight. LCMS analysis showed starting material was consumed and desired product was detected. The reaction was quenched with H₂O (50 mL) and the layers was separated. The aqueous layer was extracted with DCM (30 mL×3). The combined organics were washed with brine (30 mL), dried over anhydrous Na₂SO₄ and concentrated. The residue was purified by silica column chromatography eluting with EtOAc/Pet.ether (15%, v/v) to obtain 4-bromo-5-methyl-1-(tetrahydro-2H-pyran-2-yl)-1H-indazole (10.8 g, 55%).

LCMS (ESI, m/z): [M+1]⁺=295; RT=2.158 min.

### Step 2: Synthesis of Compound 20-3

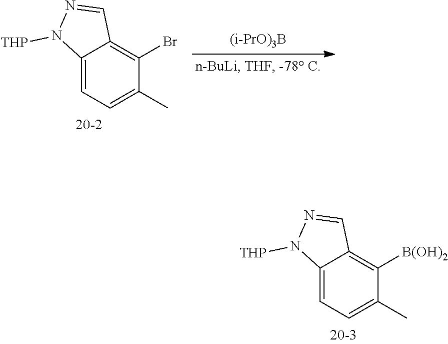

To a cooled (−78° C.) solution of 4-bromo-5-methyl-1-(tetrahydro-2H-pyran-2-yl)-1H-indazole (5.0 g, 17.00 mmol, 1.0 eq.) in anhydrous THF (30 mL) was added B (O-iPr)₃ (6.4 g, 34.00 mmol, 2.0 eq.). Then n-BuLi (2.5 mol/L in THF, 13.0 mL, 31.46 mmol, 1.85 eq.) was added dropwise to above solution over a period of 30 min, maintaining the reaction temperature between −70° C. and −65° C. After addition, the reaction was stirred at −78° C. for 3 h. LCMS analysis showed starting material was consumed and desired product formed. The reaction mixture was quenched with a solution of saturated aq.NH₄Cl (20 mL) and diluted with MTBE (30 mL). The layers was separated and the aqueous layer was extracted with MTBE (30 mL×3). The combined organics were washed with brine (50 mL), dried over anhydrous Na₂SO₄ and concentrated. The residue was dissolved in MTBE (10 mL). Petroleum ether was added dropwise to the solution at 0° C. A white solid precipitated during the petroleum ether addition. The resultant suspension was filtered and the filter cake was washed with petroleum ether (30 mL). The filter cake was dried under vacuum to obtain (5-methyl-1-(tetrahydro-2H-pyran-2-yl)-1H-indazol-4-yl)boronic acid (4.2 g, 95%).

LCMS (ESI, m/z): [M+1]⁺=261; RT=1.242 min.

### Step 3: Synthesis of Compound 20-4

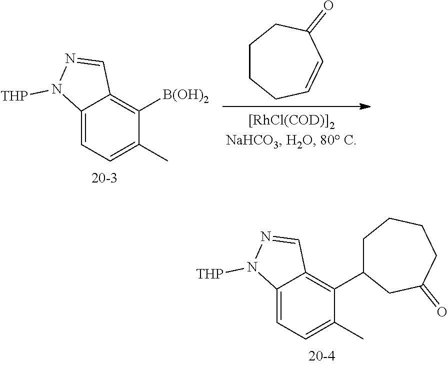

To a solution of (5-methyl-1-(tetrahydro-2H-pyran-2-yl)-1H-indazol-4-yl)boronic acid (3.0 g, 11.54 mmol, 1.0 eq.) and cyclohept-2-en-1-one (3.8 g, 34.62 mmol, 3.0 eq.) in H₂O (20 mL) was added NaHCO₃ (1.94 g, 23.08 mmol, 2.0 eq.) and [RhCl(COD)]₂ (0.28 g, 0.58 mmol, 0.05 eq.). The mixture was stirred at 80° C. under Ar overnight. LCMS analysis showed the starting material was consumed and desired product formed. The reaction mixture was diluted with EtOAc (30 mL) and the layers was separated. The aqueous layer was extracted with EtOAc (30 mL×3). The combined organics were washed with brine (30 mL), dried over anhydrous Na₂SO₄ and concentrated. The residue was purified by silica column chromatography eluting with EtOAc/Pet.ether (20%, v/v) to obtain 3-(5-methyl-1-(tetrahydro-2H-pyran-2-yl)-1H-indazol-4-yl)cycloheptan-1-one (1.3 g, 35%).

LCMS (ESI, m/z): [M+1]⁺=327; RT=1.662 min.

### Step 4: Synthesis of Compound 20-5

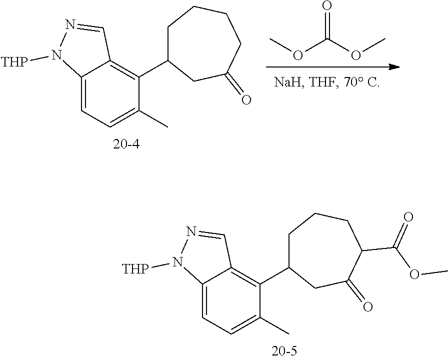

To a solution of 3-(5-methyl-1-(tetrahydro-2H-pyran-2-yl)-1H-indazol-4-yl)cycloheptan-1-one (763 mg, 2.34 mmol, 1.0 eq.) and dimethyl carbonate (4.0 mL, 46.81 mmol, 20.0 eq.) in THF (5.0 mL) was added NaH (60% dispersion in mineral oil, 140 mg, 5.85 mmol, 2.5 eq.), and the mixture was stirred at 70° C. for 2 h. LCMS analysis showed starting material was consumed and desired product formed. The reaction mixture was quenched with H₂O (10.0 mL) and extracted with EtOAc (20 mL×3). The combined organics were washed with brine (20 mL), dried over anhydrous Na₂SO₄ and concentrated. The residue was purified by silica column chromatography eluting with EtOAc/Pet.ether (20%, v/v) to obtain methyl 4-(5-methyl-1-(tetrahydro-2H-pyran-2-yl)-1H-indazol-4-yl)-2-oxocycloheptane-1-carboxylate (684 mg, 76%).

LCMS (ESI, m/z): [M+1]⁺=385; RT=1.918 min & 2.315 min

### Step 5: Synthesis of Compound 20-6

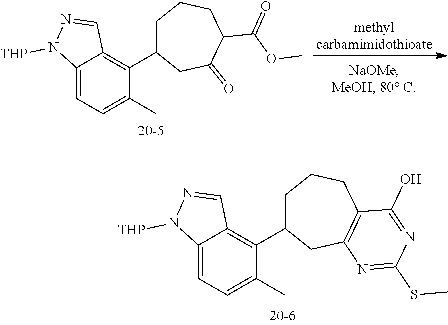

To a solution of methyl 4-(5-methyl-1-(tetrahydro-2H-pyran-2-yl)-1H-indazol-4-yl)-2-oxocycloheptane-1-carboxylate (684 mg, 1.78 mmol, 1.0 eq.) and methyl carbamimidothioate (1238 mg, 8.90 mmol, 5.0 eq.) in anhydrous MeOH (4.0 mL) was added NaOMe (962 mg, 17.8 mmol, 10.0 eq.). The reaction mixture was stirred at 80° C. under Ar overnight. LCMS analysis showed starting material was consumed and desired product formed. The reaction mixture was cooled to room temperature, diluted with water (30 mL) and extracted with EtOAc (15 mL×3). The combined organics were washed with brine (20 mL), dried over anhydrous Na₂SO₄ and concentrated. The residue was purified by silica column chromatography eluting with EtOAc/Pet.ether (60%, v/v) to obtain 8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-ol (40 mg, 5%).

LCMS (ESI, m/z): [M+1]⁺=425; RT=1.557 min.

### Step 6: Synthesis of Compound 20-7

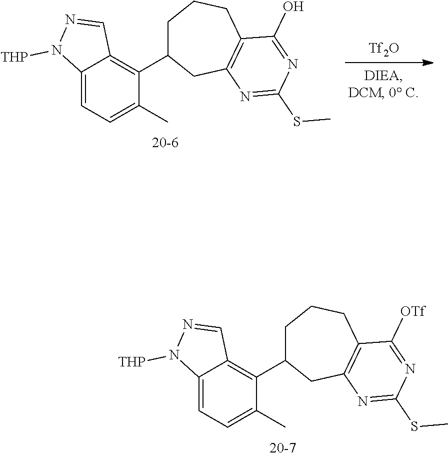

To a cooled (0° C.) solution of 8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-ol (40 mg, 0.094 mmol, 1.0 eq.) and DIEA (37 mg, 0.282 mmol, 3.0 eq.) in anhydrous DCM (3 mL) was added dropwise a solution of Tf₂O (32 mg, 0.113 mmol, 1.2 eq.) in anhydrous DCM (1 mL). The mixture was stirred at 0° C. for 1 h. LCMS analysis showed starting material was consumed and desired product formed. The reaction mixture was quenched with H₂O (2 mL) and extracted with DCM (5 mL×3). The combined organics were dried over anhydrous Na₂SO₄ and concentrated to obtain 8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-yl trifluoromethanesulfonate (50 mg, 96%), which was used directly for the next step.

LCMS (ESI, m/z): [M+1]⁺=557; RT=1.988 min.

### Step 7: Synthesis of Compound 20-8

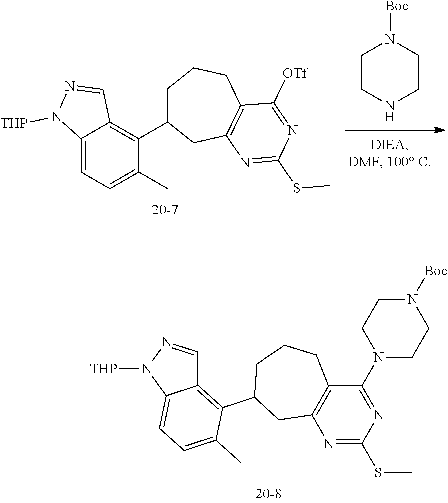

To a stirred mixture of 8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-yl trifluoromethanesulfonate (50 mg, 0.090 mmol, 1.0 eq.) and tert-butyl piperazine-1-carboxylate (33 mg, 0.180 mmol, 2.0 eq.) in anhydrous DMF (3 mL) was added DIEA (34 mg, 0.270 mmol, 3.0 eq.). The mixture was stirred at 100° C. for 2 h. LCMS analysis showed starting material was consumed and desired product formed. The reaction mixture was diluted with water (10 mL) and extracted with EtOAc (10 mL×3). The combined organics were washed with brine (5 mL), dried over anhydrous Na₂SO₄ and concentrated. The residue was purified by Prep-TLC eluting with EtOAc/Pet.ether (60%, v/v) to obtain tert-butyl 4-(8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-yl)piperazine-1-carboxylate (48 mg, 91%).

LCMS (ESI, m/z): [M+1]⁺=593; RT=1.580 min;

### Step 8: Synthesis of Compound 20-9

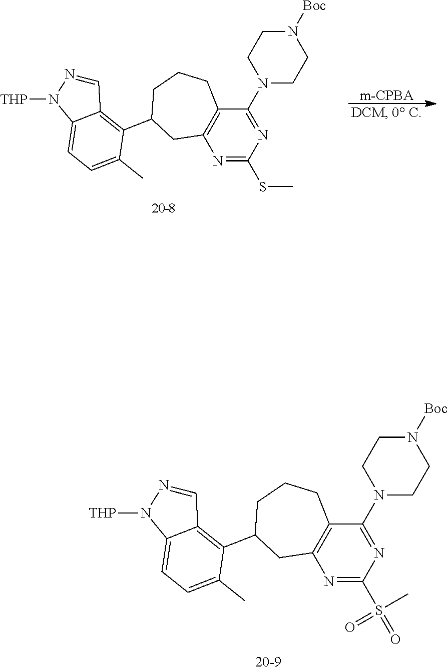

To a cooled (0° C.) solution of tert-butyl 4-(8-(5-methyl-1-(tetrahydro-2H-pyran-2-yl)-1H-indazol-4-yl)-2-(methylthio)-6,7,8,9-tetrahydro-5H-cyclohepta[d]pyrimidin-4-yl)piperazine-1-carboxylate (48 mg, 0.082 mmol, 1.0 eq.) in anhydrous DCM (3 mL) was added m-CPBA (33.47 mg, 0.165 mmol, 2.0 eq.). The mixture was stirred at 0° C. for 2 h. LCMS analysis showed starting material was consumed and desired product formed. The reaction mixture was quenched with H₂O (2 mL) and extracted with DCM (5 mL×3). The combined organics were dried over anhydrous Na₂SO₄ and concentrated to obtain tert-butyl 4-(8-(5-methyl-1-(tetrahydro-2H-pyran-2-yl)-1H-indazol-4-yl)-2-(methylsulfonyl)-6,7,8,9-tetrahydro-5H-cyclohepta[d]pyrimidin-4-yl)piperazine-1-carboxylate (77 mg, 150%), which was used directly for the next step.

LCMS (ESI, m/z): [M+1]⁺=625; RT=2.018 min;

### Step 9: Synthesis of Compound 20-10

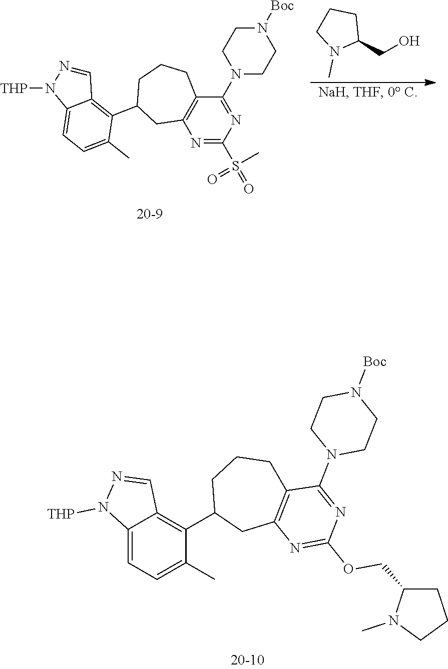

To a cooled (0° C.) solution of ((S)-1-methylpyrrolidin-2-yl)methanol (28 mg, 0.248 mmol, 2.0 eq.) in anhydrous THF (5 mL) was added NaH (60% dispersion in mineral oil, 24 mg, 0.620 mmol, 5.0 eq.). The mixture was stirred at 0° C. for 30 min. Then tert-butyl 4-(8-(5-methyl-1-(tetrahydro-2H-pyran-2-yl)-1H-indazol-4-yl)-2-(methylsulfonyl)-6,7,8,9-tetrahydro-5H-cyclohepta[d]pyrimidin-4-yl)piperazine-1-carboxylate (77 mg, 0.124 mmol, 1.0 eq.) was added and the mixture was stirred at 0° C. for 1 h. LCMS analysis showed starting material was consumed and desired product formed. The reaction mixture was quenched with H₂O (2 mL) and extracted with DCM (5 mL×3). The combined organics were dried over anhydrous Na₂SO₄ and concentrated to obtain tert-butyl 4-(8-(5-methyl-1-(tetrahydro-2H-pyran-2-yl)-1H-indazol-4-yl)-2-(((S)-1-methylpyrrolidin-2-yl)methoxy)-6,7,8,9-tetrahydro-5H-cyclohepta[d]pyrimidin-4-yl)piperazine-1-carboxylate (70 mg, 85%), which was used directly for the next step.

LCMS (ESI, m/z): [M+1]⁺=660; RT=0.962 min.

### Step 10: Synthesis of Compound 20-11

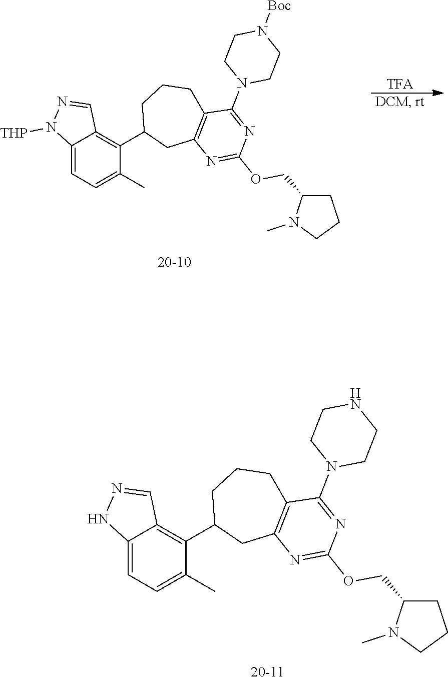

To a solution of tert-butyl 4-(8-(5-methyl-1-(tetrahydro-2H-pyran-2-yl)-1H-indazol-4-yl)-2-(((S)-1-methylpyrrolidin-2-yl)methoxy)-6,7,8,9-tetrahydro-5H-cyclohepta[d]pyrimidin-4-yl)piperazine-1-carboxylate (70 mg, 0.106 mmol, 1.0 eq.) in DCM (3 mL) was TFA (3 mL), and the mixture was stirred at room temperature for 1 h. LCMS analysis showed starting material was consumed and desired product formed. The reaction mixture was concentrated to dryness to obtain 8-(5-methyl-1H-indazol-4-yl)-2-(((S)-1-methylpyrrolidin-2-yl)methoxy)-4-(piperazin-1-yl)-6,7,8,9-tetrahydro-5H-cyclohepta[d]pyrimidine (75 mg, 150%), which was used directly for the next step.

LCMS (ESI, m/z): [M+1]⁺=476; RT=0.588 min.

### Step 11: Synthesis of Compound 20

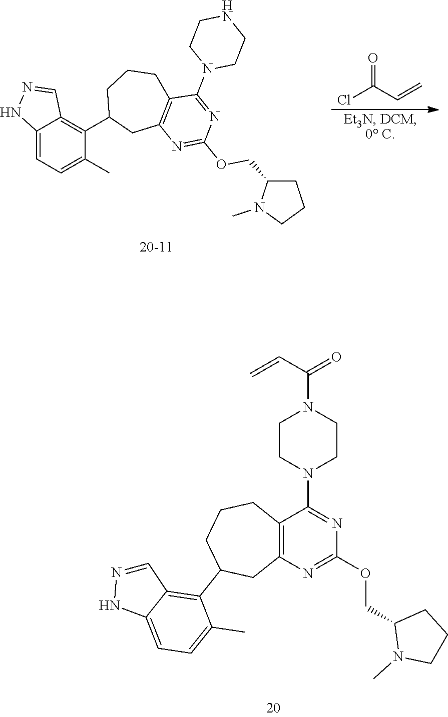

To a cooled (0° C.) solution of 8-(5-methyl-1H-indazol-4-yl)-2-(((S)-1-methylpyrrolidin-2-yl)methoxy)-4-(piperazin-1-yl)-6,7,8,9-tetrahydro-5H-cyclohepta[d]pyrimidine (75 mg, 0.158 mmol, 1.0 eq.) and Et₃N (48 mg, 0.474 mmol, 3.0 eq.) in DCM (3 mL) was added dropwise a solution of acryloyl chloride (14.29 mg, 0.158 mmol, 1.0 eq.) in DCM (1 mL). After addition, the mixture was stirred at 0° C. for 30 min. LCMS analysis showed starting material was consumed and desired product formed. Water (5 mL) was added and the organic layer was separated. The aqueous layer was extracted with DCM (5 mL×3). The combined organics were dried over anhydrous Na₂SO₄ and concentrated. The residue was purified by prep-HPLC (ACN-H₂O+0.1% NH₄HCO₃) to obtain 1-(4-(8-(5-methyl-1H-indazol-4-yl)-2-(((S)-1-methylpyrrolidin-2-yl)methoxy)-6,7,8,9-tetrahydro-5H-cyclohepta[d]pyrimidin-4-yl)piperazin-1-yl)prop-2-en-1-one (2.42 mg, 2.9%, 20) (C₃₀H₃₉N₇O₂).

LCMS (ESI, m/z): [M+1]⁺=530; RT=1.493 min;

¹H NMR (400 MHz, CDCl₃) δ 8.20 (s, 1H), 7.25 (s, 1H), 7.22-7.07 (m, 1H), 6.70-6.52 (m, 1H), 6.43-6.25 (m, 1H), 5.84-5.66 (m, 1H), 4.96 (s, 1H), 4.51 (s, 1H), 3.97-3.67 (m, 4H), 3.60-3.19 (m, 5H), 3.16-2.59 (m, 6H), 2.48-1.96 (m, 8H), 1.65 (s, 3H), 1.59-1.05 (m, 5H).

### Example 21

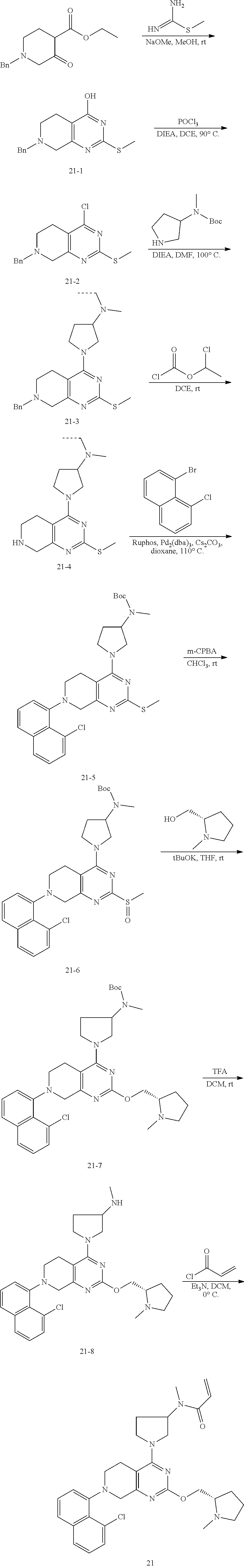

### 4. Step 1: Synthesis of Compound 21-1

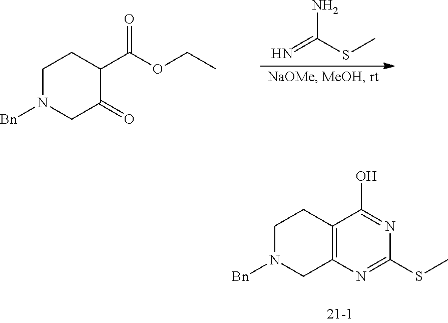

To a solution of ethyl 1-benzyl-3-oxopiperidine-4-carboxylate (2.00 g, 7.66 mmol, 1.0 eq.) in anhydrous MeOH (25 mL) was added methyl carbamimidothioate (0.7 g, 7.66 mmol, 1.0 eq.) and NaOMe (2.1 g, 38.31 mmol, 5.0 eq.). After addition, the reaction mixture was stirred at rt for 16 h. TLC showed starting material was consumed and desired product was detected. The reaction was concentrated and dissolved by water (100 mL) and filtrated. The filtrate cake was concentrated to give 7-benzyl-2-(methylthio)-5,6,7,8-tetrahydropyrido[3,4-d]pyrimidin-4-ol (1.2 g, 54%).

LCMS (ESI, m/z): [M+1]⁺=288; RT=0.798 min.

### Step 2: Synthesis of Compound 21-2

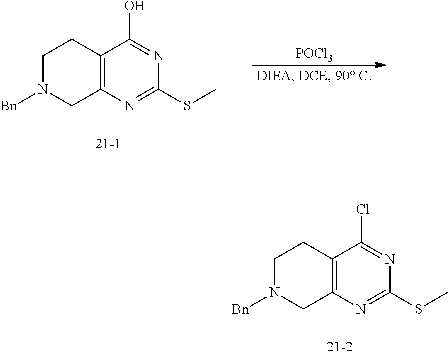 [SMILES: CSc1nc(O)c2c(n1)CN(Cc1ccccc1)CC2 ; "POCl3" ; "DIEA, DCE, 90° C." ; CSc1nc(Cl)c2c(n1)CN(Cc1ccccc1)CC2]

To a stirred mixture of 7-benzyl-2-(methylthio)-5,6,7,8-tetrahydropyrido[3,4-d]pyrimidin-4-ol (1.0 g, 3.48 mmol, 1.0 eq.) and DIEA (0.6 mL, 3.48 mmol, 1.0 eq.) in anhydrous DCE (10 mL) was added POCl₃ (5 ml, 28 mmol, 8.0 eq.). The mixture was stirred at 90° C. for 3 h. LCMS showed starting material was consumed and desired product formed. The reaction mixture was diluted with water (50 mL) and extracted with EtOAc (30 mL×3). The combined organic fractions were washed with brine (50 mL), dried over anhydrous Na₂SO₄ and concentrated to obtain 7-benzyl-4-chloro-2-(methylthio)-5,6,7,8-tetrahydropyrido[3,4-d]pyrimidine (0.8 g, 75%).

LCMS (ESI, m/z): [M+1]⁺=306; RT=2.023 min.

### Step 3: Synthesis of Compound 21-3

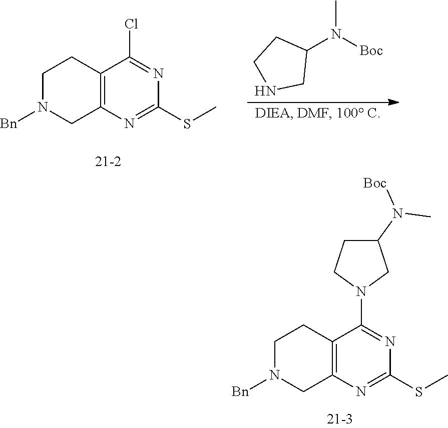

To a stirred mixture of 7-benzyl-4-chloro-2-(methylthio)-5,6,7,8-tetrahydropyrido[3,4-d]pyrimidine (500 mg, 1.64 mmol, 1.0 eq.) and DIEA (634 mg, 4.92 mmol, 3.0 eq) in anhydrous DMF (10 mL) was added tert-butyl methyl(pyrrolidin-3-yl) carbamate (328 mg, 1.64 mmol, 1.0 eq.). The mixture was stirred at 100° C. for 1 h. LCMS showed starting material was consumed and desired product formed. The reaction mixture was diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with PE/EA (1:1, v/v) to obtain tert-butyl (1-(7-benzyl-2-(methylthio)-5,6,7,8-tetrahydropyrido[3,4-d]pyrimidin-4-yl)pyrrolidin-3-yl)(methyl)carbamate (520 mg, 68%).

LCMS (ESI, m/z): [M+1]⁺=470; RT=2.064 min.

### Step 4: Synthesis of Compound 21-4

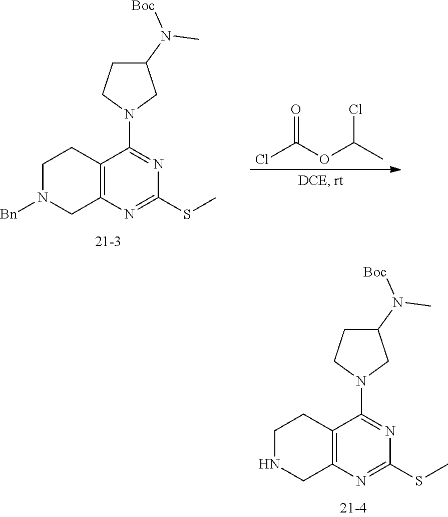

To a solution of tert-butyl (1-(7-benzyl-2-(methylthio)-5,6,7,8-tetrahydropyrido[3,4-d]pyrimidin-4-yl)pyrrolidin-3-yl)(methyl)carbamate (500 mg, 1.1 mmol, 1.0 eq.) in anhydrous DCE (16 mL) was added 1-chloroethyl carbonochloridate (305 mg, 2.1 mmol, 2.0 eq.), and the mixture was stirred at room temperature under Ar for 15 h. LCMS showed most starting material was consumed and desired product formed. The reaction mixture was concentrated and purified by silica column chromatography eluting with DCM/MeOH (10:1, v/v) to obtain tert-butyl methyl(1-(2-(methylthio)-5,6,7,8-tetrahydropyrido[3,4-d]pyrimidin-4-yl)pyrrolidin-3-yl)carbamate (180 mg, 45%).

LCMS (ESI, m/z): [M+1]⁺=380; RT=0.668 min.

### Step 5: Synthesis of Compound 21-5

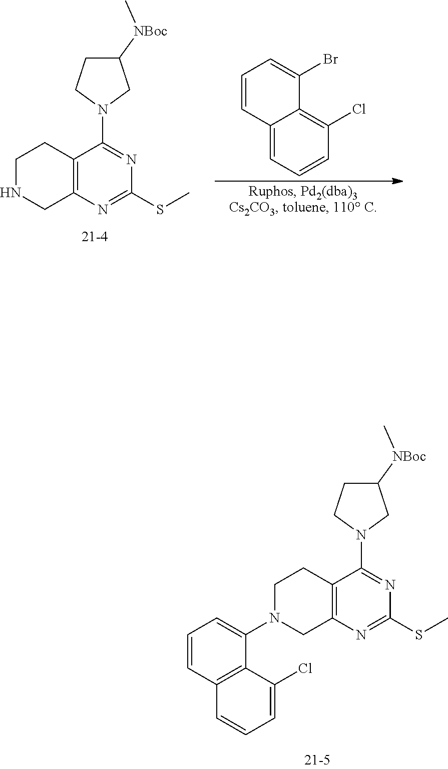

To a solution of tert-butyl methyl(1-(2-(methylthio)-5,6,7,8-tetrahydropyrido[3,4-d]pyrimidin-4-yl)pyrrolidin-3-yl)carbamate (100 mg, 0.26 mmol, 1.0 eq.) and 1-bromo-8-chloronaphthalene (190 mg, 0.79 mmol, 3.0 eq.) in Toluene (10 mL) was added CS₂CO₃ (258 mg, 0.26 mmol, 3.0 eq.), Ruphos (24 mg, 0.05 mmol, 0.2 eq.) and Pd₂(dba)₃ (36 mg, 0.04 mmol, 0.15 eq.), the mixture was stirred 110° C. under Ar for 16 h. LCMS showed starting material was consumed and desired product formed. The reaction mixture was diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with PE/EA (3:1, v/v) to obtain tert-butyl (1-(7-(8-chloronaphthalen-1-yl)-2-(methylthio)-5,6,7,8-tetrahydropyrido[3,4-d]pyrimidin-4-yl)pyrrolidin-3-yl)(methyl)carbamate (48 mg, 33%).

LCMS (ESI, m/z): [M+1]⁺=540; RT=2.100 min.

### Step 6: Synthesis of Compound 21-6

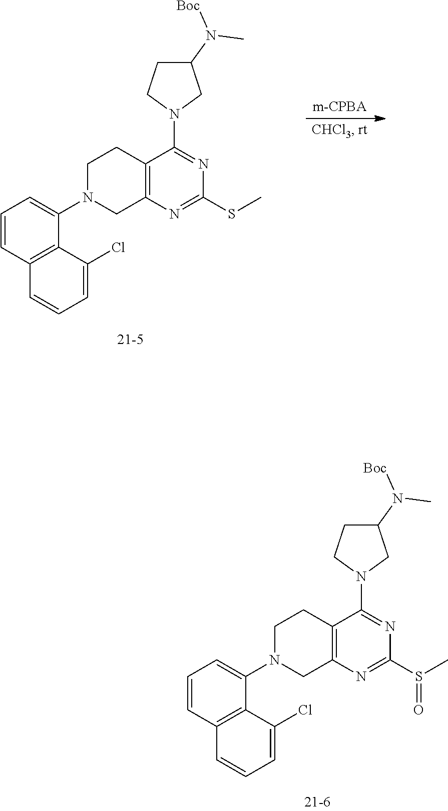 [SMILES: CSc1nc2c(c(N3CCC(N(C)C(=O)OC(C)(C)C)C3)n1)CCN(c1cccc3cccc(Cl)c13)C2 ; "CHCl3, rt" ; "m-CPBA" ; CN(C(=O)OC(C)(C)C)C1CCN(c2nc(S(C)=O)nc3c2CCN(c2cccc4cccc(Cl)c24)C3)C1]

To a solution of tert-butyl (1-(7-(8-chloronaphthalen-1-yl)-2-(methylthio)-5,6,7,8-tetrahydropyrido[3,4-d]pyrimidin-4-yl)pyrrolidin-3-yl)(methyl)carbamate (45 mg, 0.08 mmol, 1.0 eq.) in CHCl₃ (4.0 mL) was added m-CPBA (16 mg, 0.09 mmol, 1.1 eq.). The reaction mixture was stirred at rt under Ar for 0.5 h. LCMS showed starting material was consumed and desired product formed. The reaction mixture was cooled to room temperature, diluted with water (30 mL) and extracted with DCM (15 mL×3). The combined organic fractions were washed with brine (20 mL), dried over anhydrous Na₂SO₄ and concentrated. The residue was purified by silica column chromatography eluting with DCM/MeOH (15:1, v/v) to obtain tert-butyl (1-(7-(8-chloronaphthalen-1-yl)-2-(methylsulfinyl)-5,6,7,8-tetrahydropyrido[3,4-d]pyrimidin-4-yl)pyrrolidin-3-yl)(methyl)carbamate (23 mg, 50%).

LCMS (ESI, m/z): [M+1]⁺=556; RT=1.913 min.

### Step 7: Synthesis of Compound 21-7

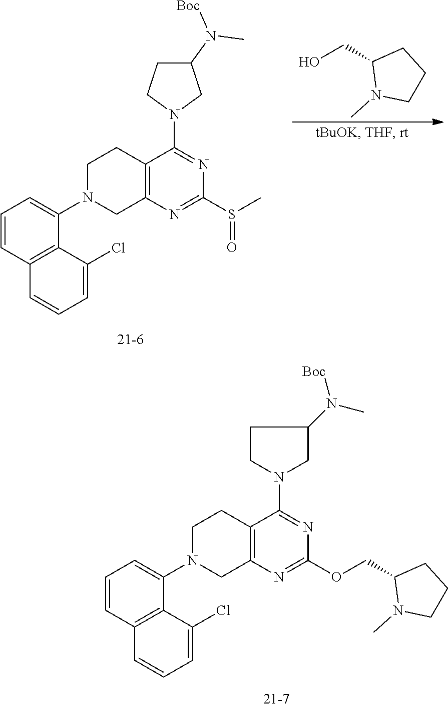

To a mixture of tert-butyl (1-(7-(8-chloronaphthalen-1-yl)-2-(methylsulfinyl)-5,6,7,8-tetrahydropyrido[3,4-d]pyrimidin-4-yl)pyrrolidin-3-yl)(methyl)carbamate (20 mg, 0.03 mmol, 1.0 eq.) in THF (5.0 mL), was added (S)-(1-methylpyrrolidin-2-yl) methanol (8.3 mg, 0.06 mmol, 2.0 eq.) and t-BuOK (4.4 mg, 0.04 mmol, 1.1 eq.). The mixture was stirred at rt for 30 min. LCMS showed starting material was consumed and desired product formed. The reaction mixture was diluted with water (30 mL) and extracted with DCM (15 mL×3). The combined organic fractions were washed with brine (20 mL). The combined organic fractions were dried over anhydrous Na₂SO₄ and concentrated. The residue was purified by prep-TLC eluting with DCM/MeOH (10:1, v/v) to obtain tert-butyl (1-(7-(8-chloronaphthalen-1-yl)-2-(((S)-1-methylpyrrolidin-2-yl)methoxy)-5,6,7,8-tetrahydropyrido[3,4-d]pyrimidin-4-yl)pyrrolidin-3-yl)(methyl)carbamate (16 mg, 72%).

### Step 8: Synthesis of Compound 21-8

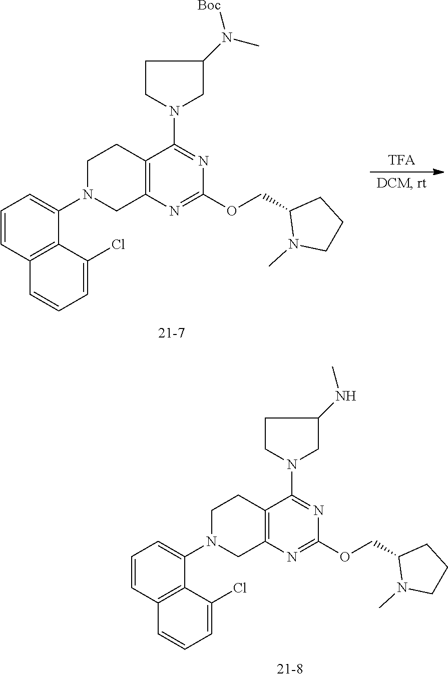 [SMILES: "TFA" ; CN(C(=O)OC(C)(C)C)C1CCN(c2nc(OC[C@@H]3CCCN3C)nc3c2CCN(c2cccc4cccc(Cl)c24)C3)C1 ; "DCM, rt" ; CNC1CCN(c2nc(OC[C@@H]3CCCN3C)nc3c2CCN(c2cccc4cccc(Cl)c24)C3)C1]

To a solution of tert-butyl (1-(7-(8-chloronaphthalen-1-yl)-2-(((S)-1-methylpyrrolidin-2-yl)methoxy)-5,6,7,8-tetrahydropyrido[3,4-d]pyrimidin-4-yl)pyrrolidin-3-yl)(methyl)carbamate (16 mg, 0.02 mmol) in DCM (1.0 mL) was TFA (1.0 mL), and the mixture was stirred at room temperature for 1 h. LCMS showed starting material was consumed and desired product formed. The reaction mixture was concentrated to dryness 1-(7-(8-chloronaphthalen-1-yl)-2-(((S)-1-methylpyrrolidin-2-yl)methoxy)-5,6,7,8-tetrahydropyrido[3,4-d]pyrimidin-4-yl)-N-methylpyrrolidin-3-amine (TFA salt, 12 mg, 90%), which was used directly for the next step.

### Step 9: Synthesis of Compound 21

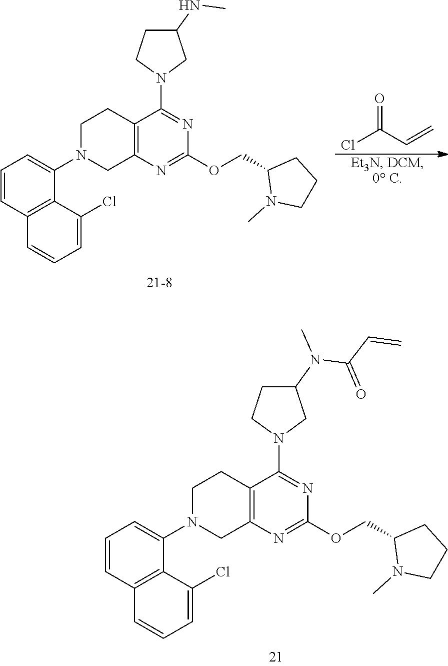

To a cooled (0° C.) solution of 1-(7-(8-chloronaphthalen-1-yl)-2-(((S)-1-methylpyrrolidin-2-yl)methoxy)-5,6,7,8-tetrahydropyrido[3,4-d]pyrimidin-4-yl)-N-methylpyrrolidin-3-amine (TFA salt, 12 mg 0.02 mmol) and Et₃N (12 mg, 0.12 mmol, 5.0 eq.) in DCM (2.5 mL) was added dropwise a solution of acryloyl chloride (2.6 mg, 0.028 mmol, 1.2 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄. The reaction was concentrated and purified by prep-HPLC (ACN-H₂O+0.1% HCOOH) to obtain N-(1-(7-(8-chloronaphthalen-1-yl)-2-(((S)-1-methylpyrrolidin-2-yl)methoxy)-5,6,7,8-tetrahydropyrido[3,4-d]pyrimidin-4-yl)pyrrolidin-3-yl)-N-methylacrylamide (HCOOH salt, 2.02 mg, 12%, 21·HCOOH) (C₃₁H₃₇ClN₆O₂·HCOOH).

LCMS (ESI, m/z): [M+1]⁺=561; RT=0.991 min;

¹H NMR (400 MHz, DMSO-d₆) δ 8.39 (s, 3H), 7.92 (d, J=8.0 Hz, 1H), 7.73 (t, J=8.5 Hz, 1H), 7.60-7.50 (m, 2H), 7.47-7.41 (m, 1H), 7.36-7.29 (m, 1H), 6.77 (s, 1H), 6.15 (d, J=17.3 Hz, 1H), 5.73 (d, J=23.2 Hz, 1H), 5.07 (s, 1H), 4.75 (s, 1H), 4.27-4.18 (m, 1H), 4.08 (d, J=17.4 Hz, 1H), 4.02-3.92 (m, 2H), 3.74 (s, 1H), 3.61 (s, 1H), 3.04-2.84 (m, 7H), 2.32 (d, J=3.7 Hz, 6H), 2.22-1.98 (m, 4H), 1.92 (d, J=8.0 Hz, 1H), 1.62 (m, 3H).

### Example 22

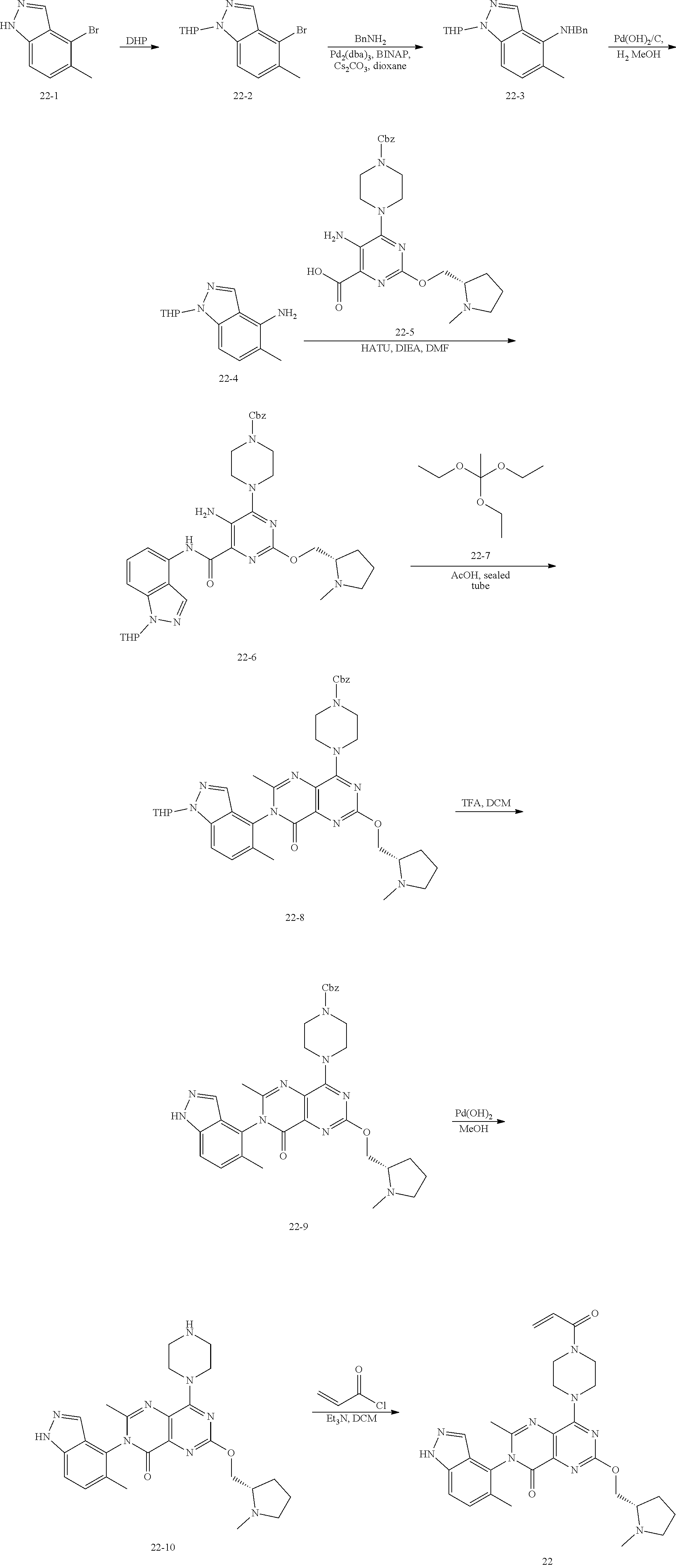

### Step 1: Synthesis of Compounds 22-2

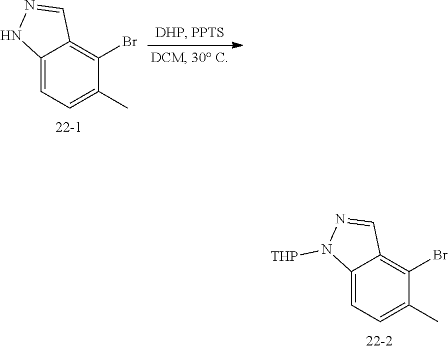

To a solution of 4-bromo-5-methyl-1H-indazole (14.0 g, 66.67 mmol, 1.0 eq.) in anhydrous DCM (30 mL) was added PPTS (1.68 g, 6.68 mmol 0.1 eq.) at room temperature. Then DHP (16.83 g, 200.02 mmol, 3 eq.) was added in one portion. The reaction mixture was stirred at 30° C. for 16 h. LCMS analysis showed starting material was consumed and desired product was detected. The reaction was quenched with H₂O (50 mL) and the layers was separated. The aqueous layer was extracted with DCM (30 mL×3). The combined organics were washed with brine (30 mL), dried over anhydrous Na₂SO₄ and concentrated. The residue was purified by silica column chromatography eluting with Pet.ether/EtOAc (15% o, v/v) to obtain 4-bromo-5-methyl-1-(tetrahydro-2H-pyran-2-yl)-1H-indazole (10.8 g, 550%).

LCMS: Rt: 2.158 min; MS m/z (ESI): 297.1 [M+3]⁺.

### Step 2: Synthesis of Compound 22-3

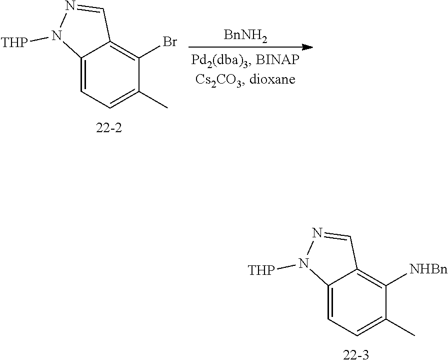

To a mixture of 4-bromo-5-methyl-1-(tetrahydro-2H-pyran-2-yl)-1H-indazole (2 g, 6.80 mmol, 1.0 eq.) in anhydrous dioxane (50 mL) was added BnNH₂ (2.18 g, 20.4 mmol 3 eq.), BINAP (423 mg, 0.68 mmol) and Cs₂CO₃ (6.63 g, 20.4 mmol), then followed by Pd₂(dba)₃ (622 mg, 0.68 mmol). The reaction mixture was stirred at 110° C. for 16 h under N₂. LCMS analysis showed starting material was consumed and desired product was detected. The reaction mixture was concentrated under reduced pressure. The residue was purified by column chromatography on silica gel eluting with petroleum ether/EtOAc (5/1-2/1, v/v) to obtain N-benzyl-5-methyl-1-(tetrahydro-2H-pyran-2-yl)-1H-indazol-4-amine (1.9 g, 87%).

LCMS: Rt: 1.813 min; MS m/z (ESI): 322.1 [M+H]⁺.

### Step 3: Synthesis of Compound 22-4

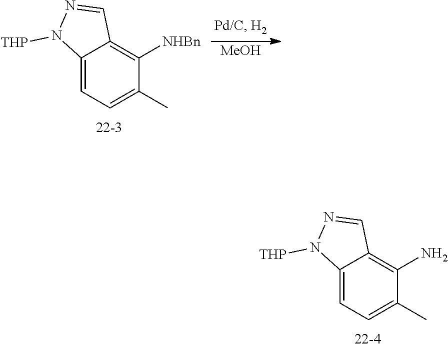

To a mixture of N-benzyl-5-methyl-1-(tetrahydro-2H-pyran-2-yl)-1H-indazol-4-amine (1.95 g, 6.07 mmol, 1.0 eq.) in anhydrous MeOH (20 mL) was added Pd/C (600 mg, 10% wt). The reaction mixture was stirred at 30° C. for 16 h under H₂ (30 psi). LCMS analysis showed starting material was consumed and desired product was detected. The mixture was filtered and the filter cake was washed with MeOH (100 mL). The filtrate was concentrated under reduced pressure to obtain 5-methyl-1-(tetrahydro-2H-pyran-2-yl)-1H-indazol-4-amine (1.28 g, 91%), which was used for the next step without further purification.

LCMS: Rt: 1.249 min; MS m/z (ESI): 232.1 [M+H]⁺.

### Step 4: Synthesis of Compound 22-6

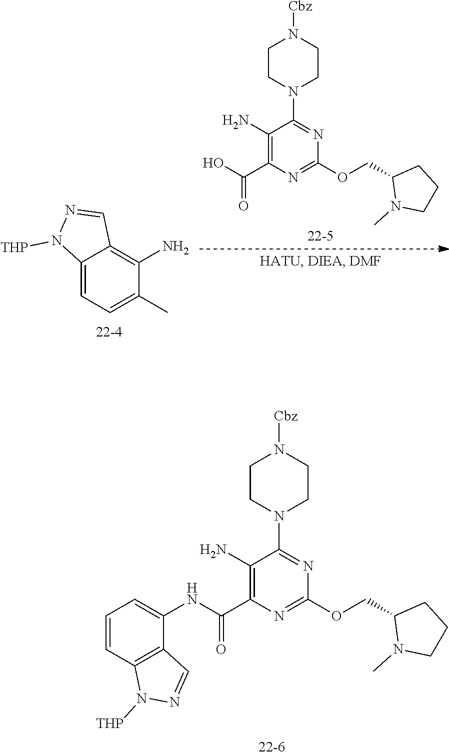

To a mixture of (S)-5-amino-6-(4-((benzyloxy)carbonyl)piperazin-1-yl)-2-((1-methylpyrrolidin-2-yl)methoxy)pyrimidine-4-carboxylic acid (160 mg, 0.34 mmol, 1.0 eq.) and 5-methyl-1-(tetrahydro-2H-pyran-2-yl)-1H-indazol-4-amine (79 mg, 0.34 mmol, 1 eq.) in anhydrous DMF (3.0 mL) was added DIEA (155 mg, 1.02 mmol, 3.0 eq.), followed by the addition of HATU (155 mg, 0.408 mmol, 1.2 eq.). The reaction mixture was stirred at 60° C. under N₂ for 1 h. LCMS showed that the reaction was completed. The reaction mixture was cooled to room temperature, diluted with EtOAc (80 mL) and washed with brine (3×80 mL), dried over anhydrous Na₂SO₄, filtered and concentrated under reduced pressure. The residue was purified by silica column chromatography eluting with DCM/MeOH (1/0-10:1, v/v) to obtain benzyl 4-(5-amino-2-(((S)-1-methylpyrrolidin-2-yl)methoxy)-6-((1-(tetrahydro-2H-pyran-2-yl)-1H-indazol-4-yl)carbamoyl)pyrimidin-4-yl)piperazine-1-carboxylate (100 mg, 26%).

LCMS: Rt: 0.946 min; MS m/z (ESI): 684.4 [M+H]⁺.

### Step 5: Synthesis of Compound 22-8

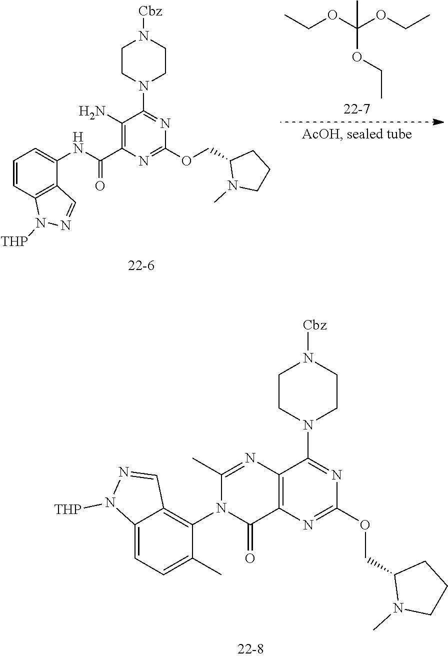

To a mixture of benzyl 4-(5-amino-2-(((S)-1-methylpyrrolidin-2-yl)methoxy)-6-((1-(tetrahydro-2H-pyran-2-yl)-1H-indazol-4-yl)carbamoyl)pyrimidin-4-yl)piperazine-1-carboxylate (45 mg, 0.0659 mmol, 1.0 eq.) in AcOH (0.5 mL) was added 1,1,1-triethoxyethane (160 mg, 0.988 mmol). The mixture was stirred at 135° C. for 8 min in a sealed tube. LCMS showed the reaction was observed. The reaction mixture was quenched with aq. NaHCO₃ solution (20 mL) to adjusted to pH=8-9 and extracted with DCM (3×20 mL). The combined organic layers were dried over anhydrous Na₂SO₄, filtered and concentrated under reduced pressure to obtain crude benzyl 4-(6-methyl-7-(5-methyl-1-(tetrahydro-2H-pyran-2-yl)-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (60 mg, >100%), which was used for the next step without further purification.

LCMS: Rt: 0.946 min; MS m/z (ESI): 708.4 [M+H]⁺.

### Step 6: Synthesis of Compound 22-9

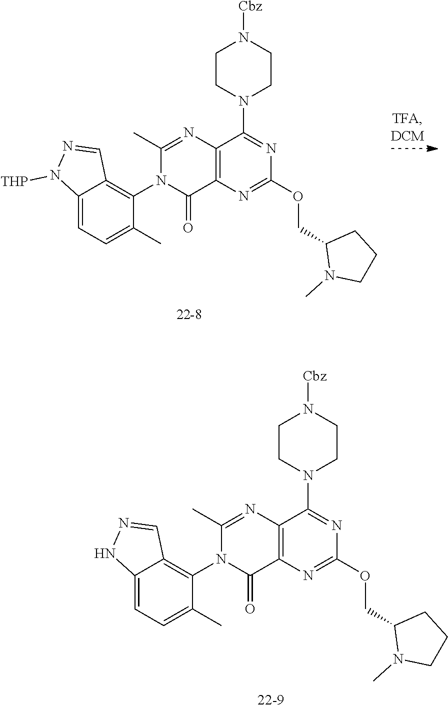

To a mixture of benzyl 4-(6-methyl-7-(5-methyl-1-(tetrahydro-2H-pyran-2-yl)-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (100 mg, 0.1415 mmol, 1 eq.) in DCM (6 mL) was added TFA (2 mL), and the mixture was stirred at 15° C. for 1 h. LCMS showed the starting material was consumed. The resulting mixture was concentrated under reduced pressure. The residue was adjusted to pH=8-9 with aq. NaHCO₃ solution and extracted with DCM (3×15 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-4-(6-methyl-7-(5-methyl-1H-indazol-4-yl)-2-((1-methylpyrrolidin-2-yl)methoxy)-8-oxo-7,8-dihydropyrimido[5,4-d]pyrimidin-4-yl)piperazine-1-carboxylate (69 mg, 78%).

LCMS: Rt: 0.887 min; MS m/z (ESI): 624.3 [M+H]⁺.

### Step 7: Synthesis of Compound 22-10

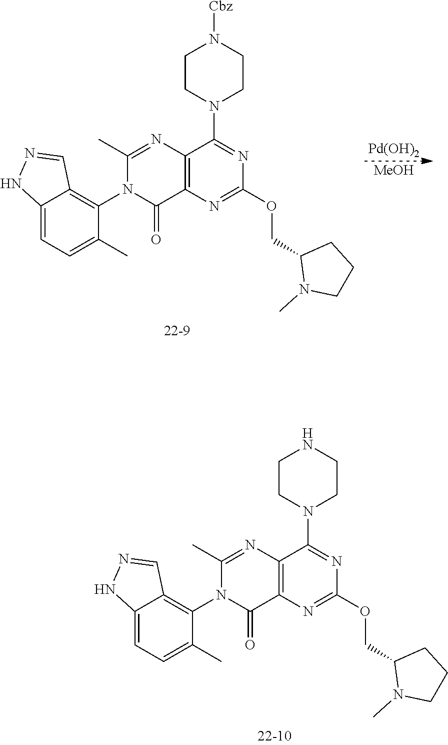 [SMILES: "MeOH" ; Cc1ccc2[nH]ncc2c1-n1c(C)nc2c(N3CCN(C(=O)OCc4ccccc4)CC3)nc(OC[C@@H]3CCCN3C)nc2c1=O ; "Pd(OH)2" ; Cc1ccc2[nH]ncc2c1-n1c(C)nc2c(N3CCNCC3)nc(OC[C@@H]3CCCN3C)nc2c1=O]

To a mixture of benzyl (S)-4-(6-methyl-7-(5-methyl-1H-indazol-4-yl)-2-((1-methylpyrrolidin-2-yl)methoxy)-8-oxo-7,8-dihydropyrimido[5,4-d]pyrimidin-4-yl)piperazine-1-carboxylate (69 mg, 0.1107 mmol) in ⁱ PrOH (1 mL) and THE (1 mL) was added Pd(OH)₂/C (15 mg, 20% wt), and the mixture was stirred at 30° C. for 41 h under H₂ (30 psi). LCMS showed the desired MS was observed. The resulting mixture was filtered through celite. The filter cake was washed with MeOH (30 mL). The filtrate was concentrated under reduced pressure to obtain (S)-2-methyl-3-(5-methyl-1H-indazol-4-yl)-6-((1-methylpyrrolidin-2-yl)methoxy)-8-(piperazin-1-yl)pyrimido[5,4-d]pyrimidin-4(3H)-one (50 mg, 92%), which was used directly for the next step without further purification.

LCMS: Rt: 0.311 min; MS m/z (ESI): 490.3 [M+H]⁺.

### Step 8: Synthesis of Compound 22

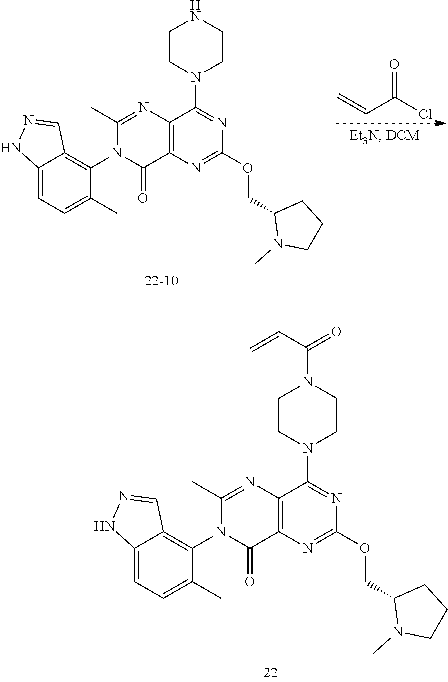

To a mixture of (S)-2-methyl-3-(5-methyl-1H-indazol-4-yl)-6-((1-methylpyrrolidin-2-yl)methoxy)-8-(piperazin-1-yl)pyrimido[5,4-d]pyrimidin-4(3H)-one (50 mg, 0.1022 mmol, 1.0 eq.) and Et₃N (31 mg, 0.3066 mmol, 3.0 eq.) in DCM (1 mL) and THE (1 mL) was added dropwise a solution of acryloyl chloride (7.5 mg, 0.0818 mmol, 0.8 eq.) in DCM (0.2 mL) at −20° C. After addition, the mixture was stirred at −20° C. for 30 min under N₂. LCMS showed the desired MS was observed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain (S)-8-(4-acryloylpiperazin-1-yl)-2-methyl-3-(5-methyl-1H-indazol-4-yl)-6-((1-methylpyrrolidin-2-yl)methoxy)pyrimido[5,4-d]pyrimidin-4(3H)-one (HCOOH salt, 2.5 mg, 4.5%, 22).

LCMS: Rt: 0.768 min; MS m/z (ESI): 544.2 [M+H]⁺;

¹H NMR (400 MHz, DMSO) δ 13.35 (s, 1H), 8.33 (s, 1.85H), 7.96 (s, 1H), 7.64 (d, J=8.5 Hz, 1H), 7.42 (d, J=8.6 Hz, 1H), 6.86 (dd, J=16.7, 10.4 Hz, 1H), 6.17 (dd, J=16.7, 2.2 Hz, 1H), 5.73 (dd, J=10.5, 2.2 Hz, 1H), 4.36-4.25 (m, 4H), 4.15-4.11 (m, 1H), 3.83-3.70 (m, 5H), 2.97-2.91 (m, 1H), 2.59-2.54 (m, 1H), 2.36 (s, 3H), 2.23-2.15 (m, 1H), 2.12 (s, 3H), 2.01 (s, 3H), 1.97-1.90 (m, 1H), 1.71-1.59 (m, 3H).

### Example 23

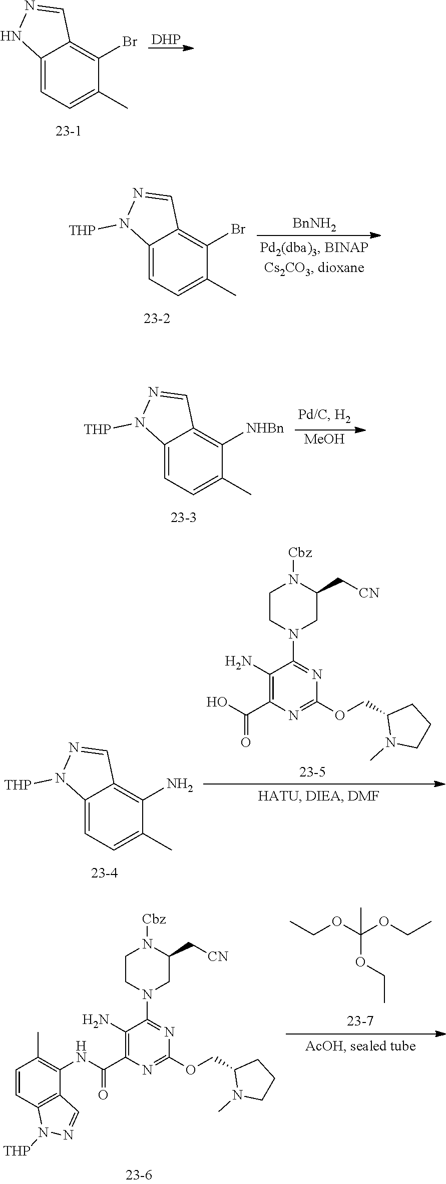

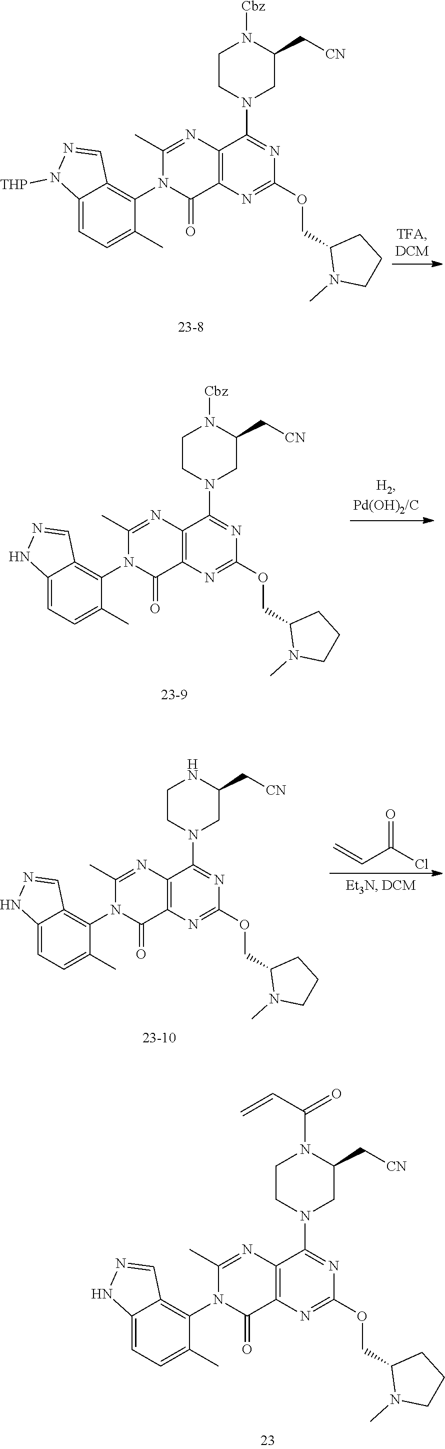

### Step 1: Synthesis of Compound 23-2

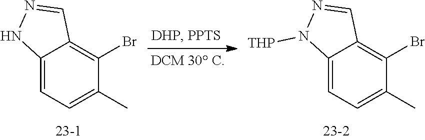

To a solution of 4-bromo-5-methyl-1H-indazole (14.0 g, 66.67 mmol, 1.0 eq.) in anhydrous DCM (30 mL) was added PPTS (1.68 g, 6.68 mmol 0.1 eq.) at room temperature. Then DHP (16.83 g, 200.02 mmol, 3 eq.) was added in one portion. The reaction mixture was stirred at 30° C. for 16 h. LCMS analysis showed starting material was consumed and desired product was detected. The reaction was quenched with H₂O (50 mL) and the layers was separated. The aqueous layer was extracted with DCM (30 mL×3). The combined organics were washed with brine (30 mL), dried over anhydrous Na₂SO₄ and concentrated. The residue was purified by silica column chromatography eluting with Pet.ether/EtOAc (15%, v/v) to obtain 4-bromo-5-methyl-1-(tetrahydro-2H-pyran-2-yl)-1H-indazole (10.8 g, 55%).

LCMS: Rt: 2.158 min; MS m/z (ESI): 297.1 [M+3]*.

### Step 2: Synthesis of Compound 23-3

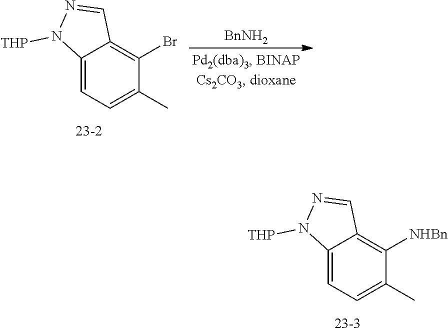

To a mixture of 4-bromo-5-methyl-1-(tetrahydro-2H-pyran-2-yl)-1H-indazole (2 g, 6.80 mmol, 1.0 eq.) in anhydrous dioxane (50 mL) was added BnNH₂ (2.18 g, 20.4 mmol 3 eq.), BINAP (423 mg, 0.68 mmol) and Cs₂CO₃ (6.63 g, 20.4 mmol), then followed by Pd₂(dba)₃ (622 mg, 0.68 mmol). The reaction mixture was stirred at 110° C. for 16 h under N₂. LCMS analysis showed starting material was consumed and desired product was detected. The reaction mixture was concentrated under reduced pressure. The residue was purified by column chromatography on silica gel eluting with petroleum ether/EtOAc (5/1-2/1, v/v) to obtain N-benzyl-5-methyl-1-(tetrahydro-2H-pyran-2-yl)-1H-indazol-4-amine (1.9 g, 87%).

LCMS: Rt: 1.813 min; MS m/z (ESI): 322.1 [M+H]⁺.

### Step 3: Synthesis of Compound 23-4

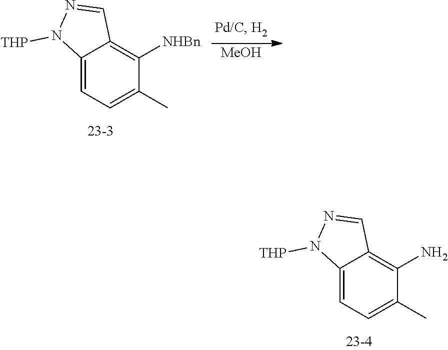

To a mixture of N-benzyl-5-methyl-1-(tetrahydro-2H-pyran-2-yl)-1H-indazol-4-amine (1.95 g, 6.07 mmol, 1.0 eq.) in anhydrous MeOH (20 mL) was added Pd/C (600 mg, 10% wt). The reaction mixture was stirred at 30° C. for 16 h under H₂ (30 psi). LCMS analysis showed starting material was consumed and desired product was detected. The mixture was filtered and the filter cake was washed with MeOH (100 mL). The filtrate was concentrated under reduced pressure to obtain 5-methyl-1-(tetrahydro-2H-pyran-2-yl)-1H-indazol-4-amine (1.28 g, 91%), which was used for the next step without further purification.

LCMS: Rt: 1.249 min; MS m/z (ESI): 232.1 [M+H]⁺.

### Step 4: Synthesis of Compound 23-6

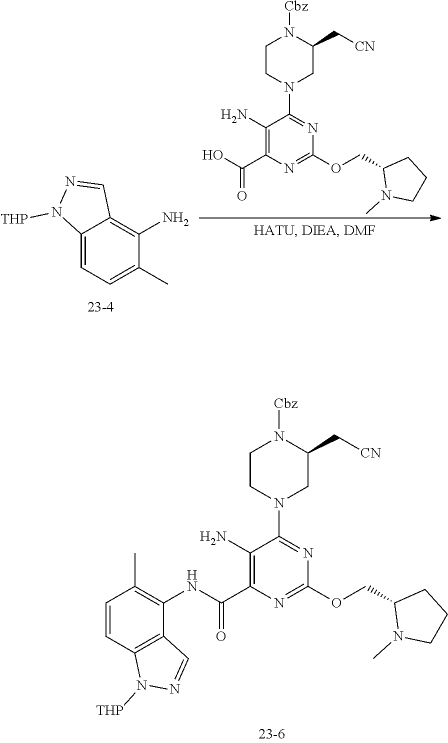

To a mixture of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (250 mg, 0.491 mmol, 1.0 eq.) and 5-methyl-1-(tetrahydro-2H-pyran-2-yl)-1H-indazol-4-amine (79 mg, 0.344 mmol, 0.7 eq.) in anhydrous DMF (5.0 mL) was added DIEA (190 mg, 1.493 mmol, 3.0 eq.), followed by the addition of HATU (186 mg, 0.491 mmol, 1.0 eq.). The reaction mixture was stirred at 60° C. under Ar for 1 h. LCMS showed starting material was consumed and desired product was formed. The reaction mixture was cooled to room temperature, diluted with EtOAc (50 mL) and washed with brine (3×50 mL), dried over anhydrous Na₂SO₄, filtered and concentrated under reduced pressure. The residue was purified by silica column chromatography eluting with DCM/MeOH (1/0-10:1, v/v) to obtain benzyl (2S)-4-(5-amino-6-((5-methyl-1-(tetrahydro-2H-pyran-2-yl)-1H-indazol-4-yl)carbamoyl)-2-(((S)-1-methylpyrrolidin-2-yl)methoxy)pyrimidin-4-yl)-2-(cyanomethyl)piperazine-1-carboxylate (120 mg, 34%).

LCMS: Rt: 0.937 min; MS m/z (ESI): 723.3 [M+H]⁺.

### Step 5: Synthesis of Compound 23-8

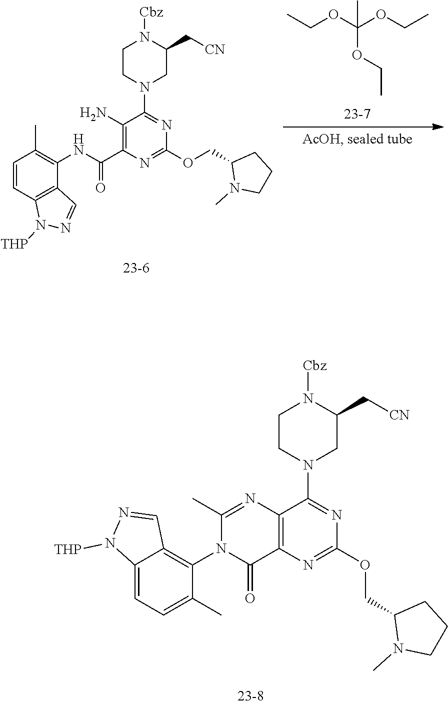

To a mixture of benzyl (2S)-4-(5-amino-6-((5-methyl-1-(tetrahydro-2H-pyran-2-yl)-1H-indazol-4-yl)carbamoyl)-2-(((S)-1-methylpyrrolidin-2-yl)methoxy)pyrimidin-4-yl)-2-(cyanomethyl)piperazine-1-carboxylate (100 mg, 0.138 mmol, 1.0 eq.) in AcOH (0.75 mL) was added 1,1,1-triethoxyethane (342 mg, 2.077 mmol). The mixture was stirred at 135° C. for 7 min in a sealed tube. LCMS showed the reaction was observed. The reaction mixture was quenched with aq. NaHCO₃ solution (20 mL) to adjusted to pH=8-9 and extracted with DCM (3×20 mL). The combined organic layers were dried over anhydrous Na₂SO₄, filtered and concentrated under reduced pressure to obtain crude benzyl (2S)-2-(cyanomethyl)-4-(6-methyl-7-(5-methyl-1-(tetrahydro-2H-pyran-2-yl)-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (124 mg, 100%), which was used for the next step without further purification.

LCMS: Rt: 0.951 min; MS m/z (ESI): 747.4 [M+H]⁺.

### Step 6: Synthesis of Compound 23-9

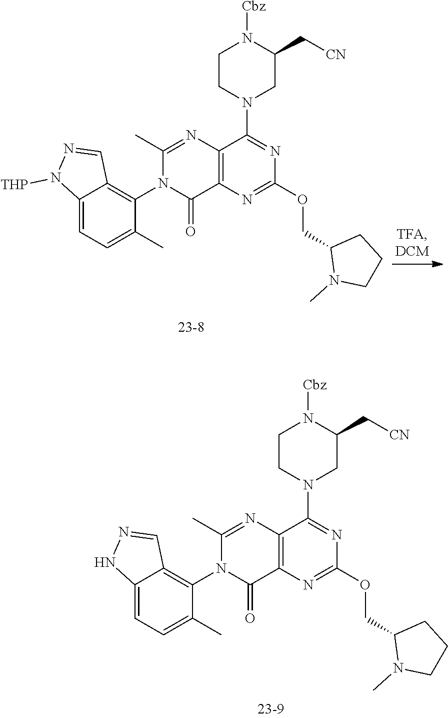

To a mixture of benzyl (2S)-2-(cyanomethyl)-4-(6-methyl-7-(5-methyl-1-(tetrahydro-2H-pyran-2-yl)-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (124 mg, 0.166 mmol, 1 eq.) in DCM (6 mL) was added TFA (2 mL), and the mixture was stirred at 15° C. for 3 h. LCMS showed the starting material was consumed. The resulting mixture was concentrated under reduced pressure. The residue was adjusted to pH=8-9 with aq. NaHCO₃ solution and extracted with DCM (3×15 mL). The combined organic layers were dried over anhydrous Na₂SO₄, filtered and concentrated under reduced pressure. The residue was purified by prep-TLC eluting with DCM/MeOH (10:1, v/v) to obtain benzyl (S)-2-(cyanomethyl)-4-(6-methyl-7-(5-methyl-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (73 mg, 66%).

LCMS: Rt: 0.864 min; MS m/z (ESI): 663.3 [M+H]⁺.

### Step 7: Synthesis of Compound 23-10

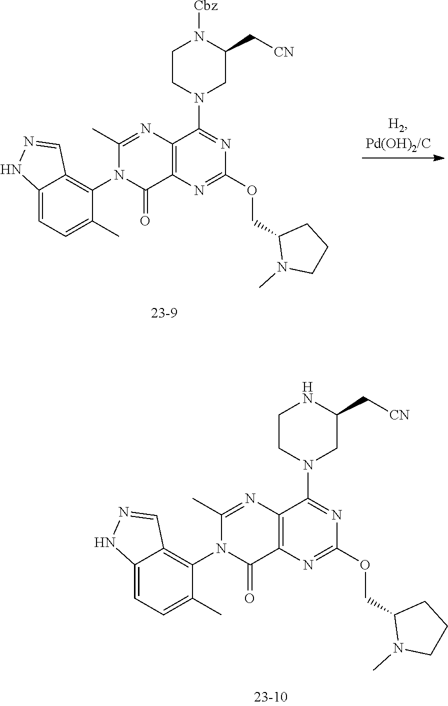 [SMILES: Cc1ccc2[nH]ncc2c1-n1c(C)nc2c(N3CCN(C(=O)OCc4ccccc4)[C@@H](CC#N)C3)nc(OC[C@@H]3CCCN3C)nc2c1=O ; Cc1ccc2[nH]ncc2c1-n1c(C)nc2c(N3CCN[C@@H](CC#N)C3)nc(OC[C@@H]3CCCN3C)nc2c1=O]

To a mixture of benzyl (S)-2-(cyanomethyl)-4-(6-methyl-7-(5-methyl-1H-indazol-4-yl)-2-(((S)-1-methylpyrrolidin-2-yl)methoxy)-8-oxo-7,8-dihydropyrimido[5,4-d]pyrimidin-4-yl)piperazine-1-carboxylate (73 mg, 0.110 mmol) in MeOH (2 mL) was added Pd(OH)₂/C (20 mg, 20% wt), and the mixture was stirred at room temperature for 1 h under H₂ (50 psi). LCMS showed starting material was consumed and desired product formed. The resulting mixture was filtered through celite. The filter cake was washed with MeOH (50 mL). The filtrate was concentrated under reduced pressure to obtain 2-((S)-4-(6-methyl-7-(5-methyl-1H-indazol-4-yl)-2-(((S)-1-methylpyrrolidin-2-yl)methoxy)-8-oxo-7,8-dihydropyrimido[5,4-d]pyrimidin-4-yl)piperazin-2-yl)acetonitrile (45 mg, 77%), which was used directly for the next step without further purification.

LCMS: Rt: 0.934 min; MS m/z (ESI): 529.2 [M+H]⁺.

### Step 8: Synthesis of Compound 23

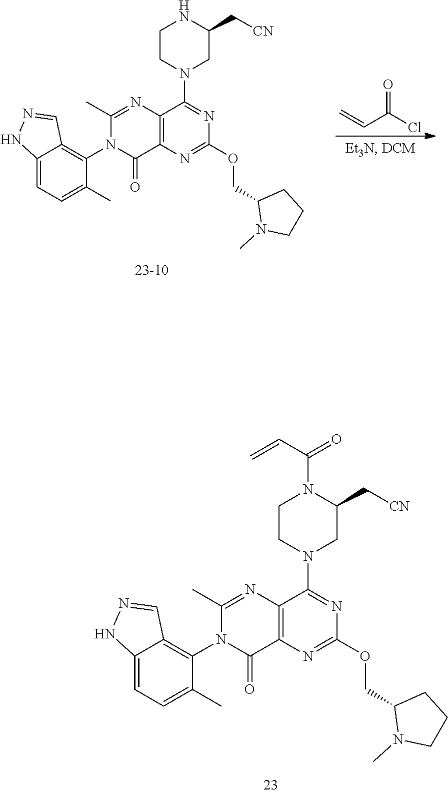

To a mixture of 2-((S)-4-(6-methyl-7-(5-methyl-1H-indazol-4-yl)-2-(((S)-1-methylpyrrolidin-2-yl)methoxy)-8-oxo-7,8-dihydropyrimido[5,4-d]pyrimidin-4-yl)piperazin-2-yl)acetonitrile (45 mg, 0.0852 mmol, 1.0 eq.) and Et₃N (26 mg, 0.2556 mmol, 3.0 eq.) in DCM (3 mL) was added dropwise a solution of acryloyl chloride (7.7 mg, 0.0852 mmol, 1.0 eq.) in DCM (0.2 mL) at −20° C. After addition, the mixture was stirred at −20° C. for 30 min under N₂. LCMS showed starting material was consumed and desired product formed. The mixture was quenched with water (10 mL) and extracted with DCM (3×10 mL). The combined organic layers were dried over anhydrous Na₂SO₄, filtered and concentrated under reduced pressure. The residue was purified by HCOOH prep-HPLC separation to obtain 2-((S)-1-acryloyl-4-(6-methyl-7-(5-methyl-1H-indazol-4-yl)-2-(((S)-1-methylpyrrolidin-2-yl)methoxy)-8-oxo-7,8-dihydropyrimido[5,4-d]pyrimidin-4-yl)piperazin-2-yl)acetonitrile (HCOOH salt, 4.0 mg, 8%, 23).

LCMS: Rt: 0.823 min; MS m/z (ESI): 583.3 [M+H]⁺;

¹H NMR (400 MHz, DMSO) δ 13.37 (s, 1H), 8.33 (s, 1.92H), 7.92 (d, J=12.4 Hz, 1H), 7.64 (d, J=8.5 Hz, 1H), 7.43 (d, J=8.3 Hz, 1H), 7.02-6.76 (m, 1H), 6.20 (d, J=18.2 Hz, 1H), 5.79 (d, J=10.3 Hz, 1H), 5.63-4.73 (m, 3H), 4.49-4.30 (m, 1.5H), 4.17-4.11 (m, 1.5H), 3.78-3.64 (m, 2H), 3.14-2.93 (m, 4H), 2.60-2.52 (m, 1H), 2.36 (d, J=1.0 Hz, 3H), 2.23-2.15 (m, 1H), 2.12 (d, J=5.9 Hz, 3H), 2.03 (s, 3H), 1.98-1.89 (m, 1H), 1.71-1.59 (m, 3H).

### Example 24

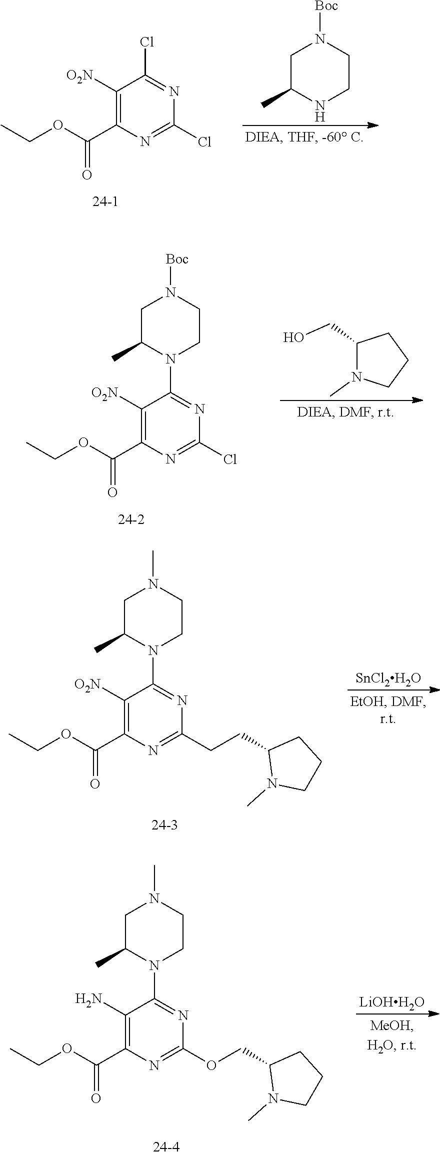

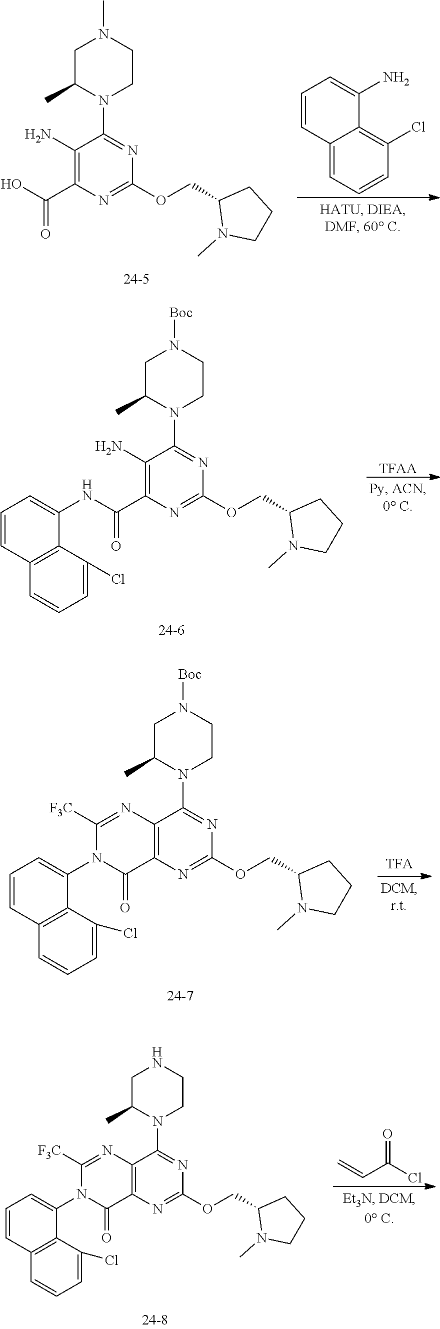

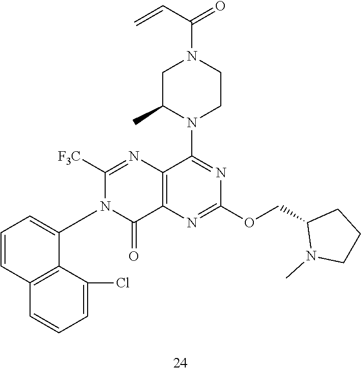 [SMILES: C=CC(=O)N1CCN(c2nc(OC[C@@H]3CCCN3C)nc3c(=O)n(-c4cccc5cccc(Cl)c45)c(C(F)(F)F)nc23)[C@@H](C)C1]

### Step 1: Synthesis of Compound 24-2

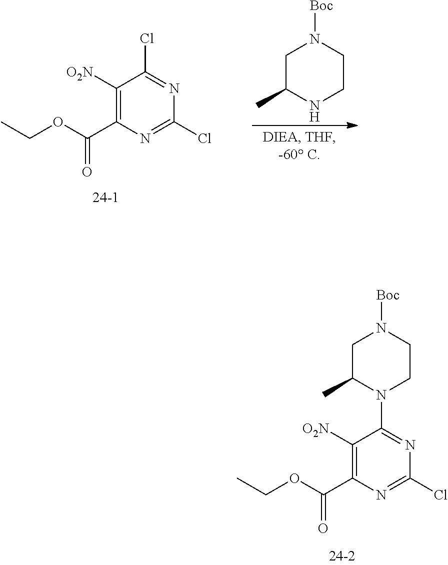 [SMILES: CCOC(=O)c1nc(Cl)nc(Cl)c1[N+](=O)[O-] ; C[C@H]1CN(C(=O)OC(C)(C)C)CCN1 ; CCOC(=O)c1nc(Cl)nc(N2CCN(C(=O)OC(C)(C)C)C[C@@H]2C)c1[N+](=O)[O-]]

To a cooled (−60° C.) solution of ethyl 2,6-dichloro-5-nitropyrimidine-4-carboxylate (5.0 g, 0.019 mol, 1.0 eq.) in anhydrous THF (50 mL) was added dropwise a solution of tert-butyl (S)-3-methylpiperazine-1-carboxylate (3.75 g, 0.019 mol, 1.0 eq.) and DIEA (4.6 mL, 0.028 mol, 1.5 eq.) in anhydrous THF (30 mL). The mixture was stirred at −60° C. under Ar for 1 h. LCMS showed starting material was consumed and desired product formed. The reaction mixture was concentrated and the residue was purified by silica column chromatography eluting with Pet. ether/EtOAc (3:1, v/v) to obtain ethyl (S)-6-(4-(tert-butoxycarbonyl)-2-methylpiperazin-1-yl)-2-chloro-5-nitropyrimidine-4-carboxylate (8.2 g, crude).

LCMS (ESI, m/z): [M+1]⁺=430; RT=2.141 min.

### Step 2: Synthesis of Compound 24-3

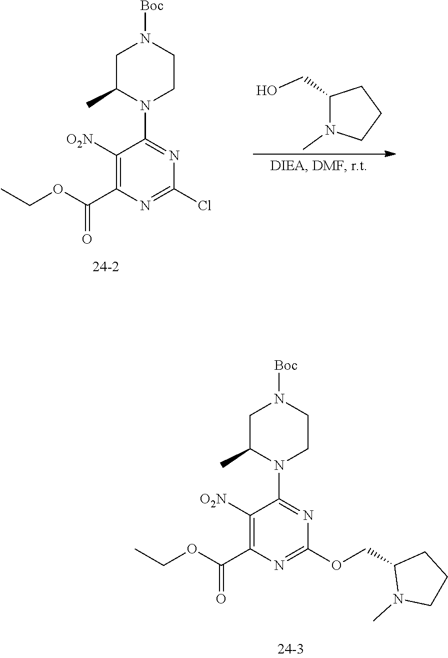

To a solution of ethyl (S)-6-(4-(tert-butoxycarbonyl)-2-methylpiperazin-1-yl)-2-chloro-5-nitropyrimidine-4-carboxylate (8.2 g, 0.019 mol, 1.0 eq.) and DIEA (6.3 ml, 0.038 mol, 2.0 eq.) in anhydrous DMF (60.0 mL) was added (S)-(1-methylpyrrolidin-2-yl) methanol (3.3 g, 0.029 mol, 1.5 eq.). The mixture was stirred at room temperature for 16 h. LCMS showed starting material was consumed and desired product formed. The reaction mixture was diluted with H₂O (100 mL) and extracted with EtOAc (80 mL×3). The combined organic fractions were washed with brine (100 mL), dried over anhydrous Na₂SO₄ and concentrated. The residue was purified by silica column chromatography eluting with DCM/MeOH (15:1, v/v) to obtain ethyl 6-((S)-4-(tert-butoxycarbonyl)-2-methylpiperazin-1-yl)-2-(((S)-1-methylpyrrolidin-2-yl) methoxy)-5-nitropyrimidine-4-carboxylate (8.8 g, 91% yield).

LCMS (ESI, m/z): [M+1]⁺=509; RT=1.099 min.

### Step 3: Synthesis of Compound 24-4

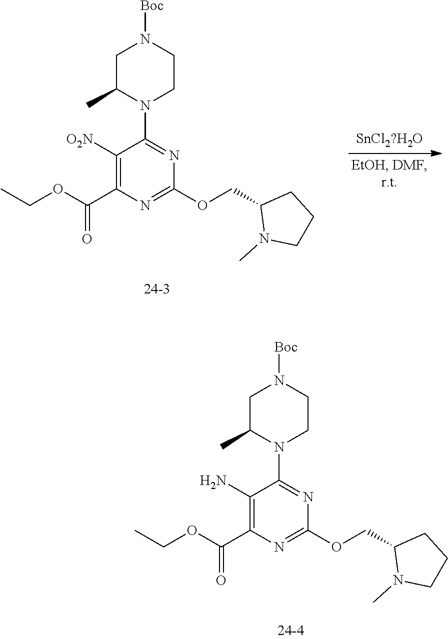 [SMILES: CCOC(=O)c1nc(OC[C@@H]2CCCN2C)nc(N2CCN(C(=O)OC(C)(C)C)C[C@@H]2C)c1[N+](=O)[O-] ; CCOC(=O)c1nc(OC[C@@H]2CCCN2C)nc(N2CCN(C(=O)OC(C)(C)C)C[C@@H]2C)c1N]

To a solution of ethyl 6-((S)-4-(tert-butoxycarbonyl)-2-methylpiperazin-1-yl)-2-(((S)-1-methylpyrrolidin-2-yl) methoxy)-5-nitropyrimidine-4-carboxylate (8.8 g, 0.017 mol, 1.0 eq.) in anhydrous DMF (20 mL)/EtOH (60 mL) was added SnCl₂·2H₂O (19.6 g, 0.087 mol, 5.0 eq.). The mixture was stirred at room temperature under Ar for 16 h. LCMS showed that starting material was consumed and desired product formed. The reaction mixture was concentrated to remove EtOH and then diluted with EtOAc (120 mL), followed by the addition of aq. NaHCO₃ (sat. 180 mL). The resulting mixture was filtered through celite. The organic layer of the filtrate was separated and the aqueous layer was extracted with EtOAc (100 mL×2). The combined organic fractions were washed with brine (160 mL), dried over anhydrous Na₂SO₄ and concentrated. The residue was purified by silica column chromatography eluting with DCM/MeOH (15:1, v/v) to obtain ethyl 5-amino-6-((S)-4-(tert-butoxycarbonyl)-2-methylpiperazin-1-yl)-2-(((S)-1-methylpyrrolidin-2-yl) methoxy) pyrimidine-4-carboxylate (3.3 g, 40% yield).

LCMS (ESI, m/z): [M+1]⁺=479; RT=0.867 min.

### Step 4: Synthesis of Compound 24-5

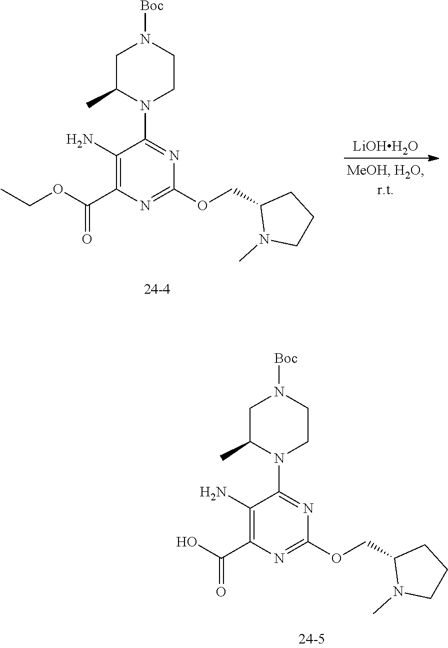 [SMILES: CCOC(=O)c1nc(OC[C@@H]2CCCN2C)nc(N2CCN(C(=O)OC(C)(C)C)C[C@@H]2C)c1N ; "LiOH·H2O" ; C[C@H]1CN(C(=O)OC(C)(C)C)CCN1c1nc(OC[C@@H]2CCCN2C)nc(C(=O)O)c1N]

To a mixture of ethyl 5-amino-6-((S)-4-(tert-butoxycarbonyl)-2-methylpiperazin-1-yl)-2-(((S)-1-methylpyrrolidin-2-yl) methoxy) pyrimidine-4-carboxylate (3.3 g, 0.007 mol, 1.0 eq.) in MeOH (60 mL) and H₂O (10 mL) was added LiOH·H₂O (1.45 g, 0.034 mol, 5.0 eq.). The mixture was stirred at room temperature for 2 h. LCMS showed starting material was consumed and desired product formed. The reaction mixture was acidified with aq. HCl (0.5 M) to pH=6, and then concentrated to dryness to obtain 5-amino-6-((S)-4-(tert-butoxycarbonyl)-2-methylpiperazin-1-yl)-2-(((S)-1-methylpyrrolidin-2-yl) methoxy) pyrimidine-4-carboxylic acid (5.06 g, crude).

LCMS (ESI, m/z): [M+1]⁺=451; RT=0.928 min.

### Step 5: Synthesis of Compound 24-6

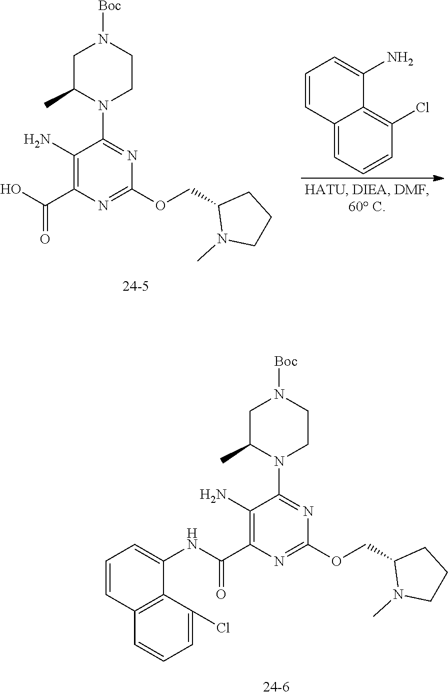 [SMILES: C[C@H]1CN(C(=O)OC(C)(C)C)CCN1c1nc(OC[C@@H]2CCCN2C)nc(C(=O)O)c1N ; Nc1cccc2cccc(Cl)c12 ; C[C@H]1CN(C(=O)OC(C)(C)C)CCN1c1nc(OC[C@@H]2CCCN2C)nc(C(=O)Nc2cccc3cccc(Cl)c23)c1N]

To a solution of 5-amino-6-((S)-4-(tert-butoxycarbonyl)-2-methylpiperazin-1-yl)-2-(((S)-1-methylpyrrolidin-2-yl) methoxy) pyrimidine-4-carboxylic acid (800 mg, 1.78 mmol, 1.0 eq.) and 8-methylnaphthalen-1-amine (220 mg, 1.24 mmol, 0.7 eq.) in anhydrous DMF (5.0 mL) was added DIEA (0.88 mL, 5.33 mmol, 3.0 eq.), followed by the addition of HATU (675 mg, 1.78 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (20 mL×3). The combined organic fractions were washed with brine (30 mL), dried over anhydrous Na₂SO₄ and concentrated. The residue was purified by silica column chromatography eluting with DCM/MeOH (15:1, v/v) to obtain tert-butyl (S)-4-(5-amino-6-((8-chloronaphthalen-1-yl) carbamoyl)-2-(((S)-1-methylpyrrolidin-2-yl) methoxy) pyrimidin-4-yl)-3-methylpiperazine-1-carboxylate (430 mg, 57% yield).

LCMS (ESI, m/z): [M+1]⁺=688.0; RT=1.406 min.

### Step 6: Synthesis of Compound 24-7

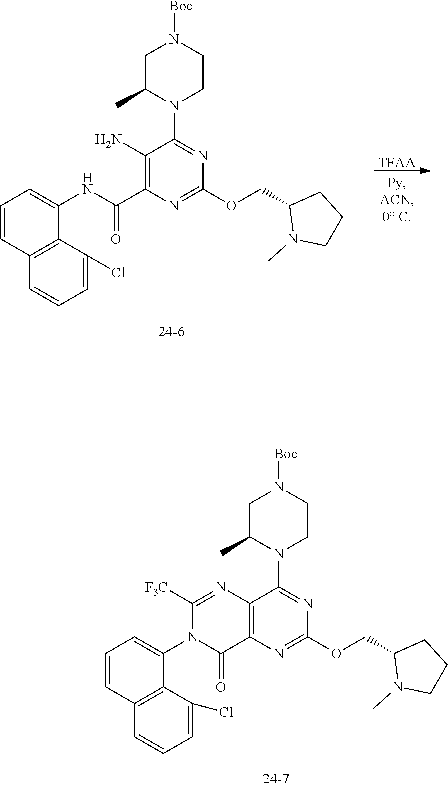 [SMILES: C[C@H]1CN(C(=O)OC(C)(C)C)CCN1c1nc(OC[C@@H]2CCCN2C)nc(C(=O)Nc2cccc3cccc(Cl)c23)c1N ; "TFAA" ; C[C@H]1CN(C(=O)OC(C)(C)C)CCN1c1nc(OC[C@@H]2CCCN2C)nc2c(=O)n(-c3cccc4cccc(Cl)c34)c(C(F)(F)F)nc12]

To a cooled (0° C.) solution of tert-butyl (S)-4-(5-amino-6-((8-chloronaphthalen-1-yl) carbamoyl)-2-(((S)-1-methylpyrrolidin-2-yl) methoxy) pyrimidin-4-yl)-3-methylpiperazine-1-carboxylate (200 mg, 0.33 mmol, 1.0 eq.) in anhydrous ACN (3.0 mL) was added pyridine (259 mg, 3.28 mmol, 10.0 eq.), followed by the addition of TFAA (414 mg, 1.97 mmol, 6.0 eq.). The mixture was stirred at 0° C. for 1 h. LCMS showed starting material was consumed and desired product formed. The reaction mixture was quenched with aq. NH₄Cl (sat. 25 mL) and extracted with EtOAc (15 mL×3). The combined organic fractions were washed with brine (20 mL), dried over anhydrous Na₂SO₄ and concentrated. The residue was purified by prep-TLC eluting with DCM/MeOH (10:1, v/v) to obtain tert-butyl (S)-4-(7-(8-chloronaphthalen-1-yl)-2-(((S)-1-methylpyrrolidin-2-yl) methoxy)-8-oxo-6-(trifluoromethyl)-7,8-dihydropyrimido[5,4-d] pyrimidin-4-yl)-3-methylpiperazine-1-carboxylate (110 mg, 49% yield).

LCMS (ESI, m/z): [M+1]⁺=610; RT=1.227 min.

### Step 7: Synthesis of Compound 24-8

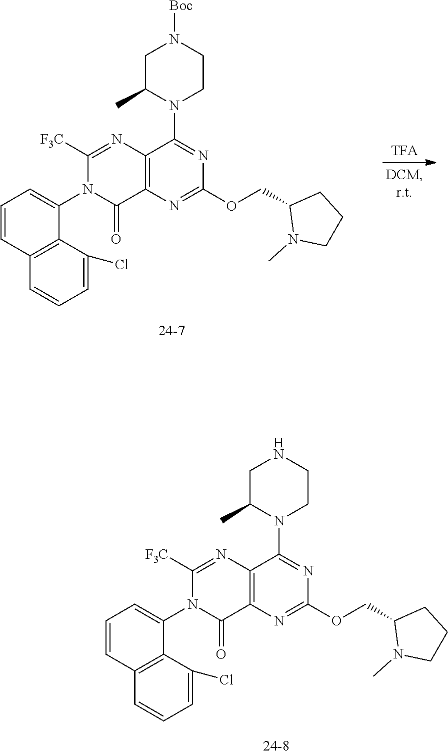 [SMILES: C[C@H]1CN(C(=O)OC(C)(C)C)CCN1c1nc(OC[C@@H]2CCCN2C)nc2c(=O)n(-c3cccc4cccc(Cl)c34)c(C(F)(F)F)nc12 ; C[C@H]1CNCCN1c1nc(OC[C@@H]2CCCN2C)nc2c(=O)n(-c3cccc4cccc(Cl)c34)c(C(F)(F)F)nc12]

To a solution of tert-butyl (S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-3-methylpiperazine-1-carboxylate (100 mg, 0.15 mmol) in anhydrous DCM (5.0 mL) was added TFA (3 mL), and the mixture was stirred at room temperature for 1 h. LCMS showed starting material was consumed and desired product formed. The reaction mixture was concentrated to obtain 3-(8-chloronaphthalen-1-yl)-8-((S)-2-methylpiperazin-1-yl)-6-(((S)-1-methylpyrrolidin-2-yl)methoxy)-2-(trifluoromethyl)-2,3-dihydropyrimido[5,4-d]pyrimidin-4(1H)-one (TFA salt, 96 mg, 94% yield), which was used directly for the next step.

LCMS (ESI, m/z): [M+1]⁺=588; RT=0.791 min.

### Step 8: Synthesis of Compounds 24-a & 24-b

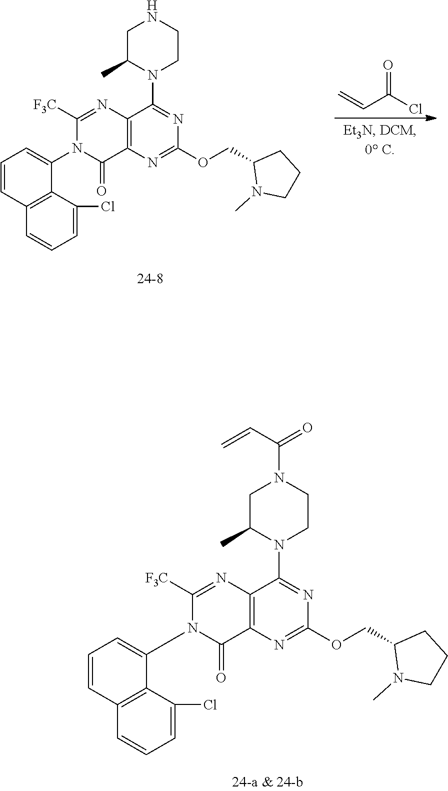

To a cooled (0° C.) solution of 3-(8-chloronaphthalen-1-yl)-8-((S)-2-methylpiperazin-1-yl)-6-(((S)-1-methylpyrrolidin-2-yl)methoxy)-2-(trifluoromethyl)-2,3-dihydropyrimido[5,4-d]pyrimidin-4(1H)-one (TFA salt, 96 mg, 0.14 mmol, 1.0 eq.) and Et₃N (71 mg, 0.70 mmol, 5.0 eq.) in anhydrous DCM (2.5 mL) was added dropwise a solution of acryloyl chloride (13 mg, 0.14 mmol, 1.0 eq.) in anhydrous DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((S)-4-acryloyl-2-methylpiperazin-1-yl)-3-(8-chloronaphthalen-1-yl)-6-(((S)-1-methylpyrrolidin-2-yl) methoxy)-2-(trifluoromethyl)pyrimido[5,4-d] pyrimidin-4(3H)-one (6.4 mg, 15% yield, 24-a), and 8-((S)-4-acryloyl-2-methylpiperazin-1-yl)-3-(8-chloronaphthalen-1-yl)-6-(((S)-1-methylpyrrolidin-2-yl) methoxy)-2-(trifluoromethyl)pyrimido[5,4-d] pyrimidin-4(3H)-one (7.4 mg, 18% yield, 24-b).

24-a:

LCMS (ESI, m/z): [M+1]⁺=642; RT=1.840 min; ¹H NMR (400 MHz, CDCl₃) δ 8.07 (d, J=8.4 Hz, 1H), 7.90 (d, J=8.0 Hz, 1H), 7.66-7.53 (m, 2H), 7.44 (d, J=6.5 Hz, 2H), 6.71-6.53 (m, 1H), 6.39 (d, J=16.8 Hz, 1H), 5.78 (d, J=10.3 Hz, 1H), 5.69-5.29 (m, 1H), 4.69-4.27 (m, 3H), 4.05-3.80 (m, 1H), 3.71-3.52 (m, 2H), 3.47-3.14 (m, 2H), 2.93 (s, 1H), 2.60 (s, 3H), 2.44 (s, 1H), 2.21-2.00 (m, 2H), 1.97-1.78 (m, 3H), 1.45-1.37 (m, 3H);

¹⁹F NMR (400 MHz, CDCl₃) δ −64.8.

24-b:

LCMS (ESI, m/z): [M+1]⁺=642; RT=1.831 min;

¹H NMR (400 MHz, CDCl₃) δ 8.06 (d, J=8.1 Hz, 1H), 7.89 (d, J=8.1 Hz, 1H), 7.66-7.53 (m, 2H), 7.49-7.37 (m, 2H), 6.68-6.51 (m, 1H), 6.39 (d, J=16.6 Hz, 1H), 5.78 (d, J=10.0 Hz, 1H), 5.69-5.28 (m, 1H), 4.74-4.29 (m, 3H), 4.07-3.79 (m, 1H), 3.69-3.49 (m, 2H), 3.44-3.15 (m, 2H), 2.85 (s, 1H), 2.55 (s, 3H), 2.42-2.33 (m, 1H), 2.28-1.97 (m, 2H), 1.95-1.75 (m, 3H), 1.48-1.37 (m, 3H);

¹⁹F NMR (400 MHz, CDCl₃) δ −64.9.

### Example 25

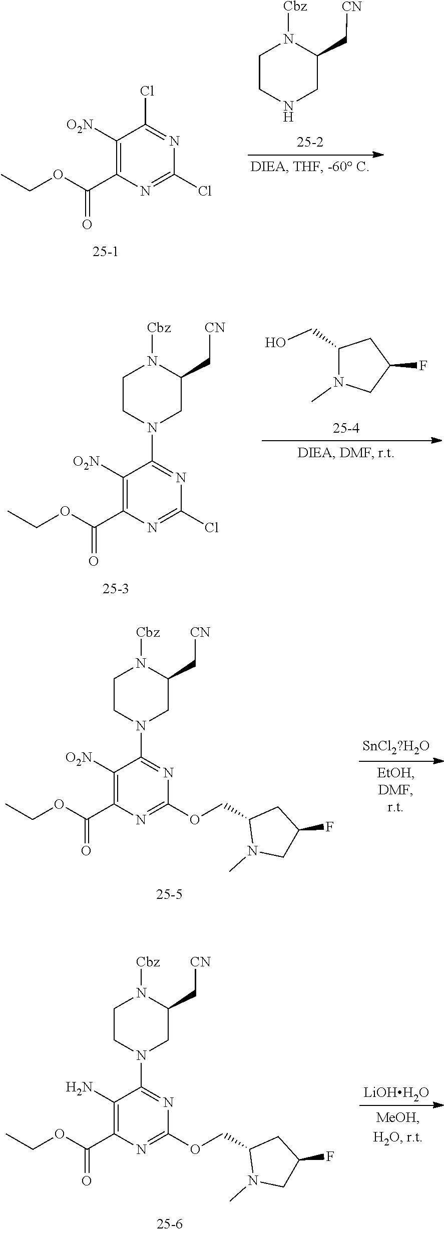

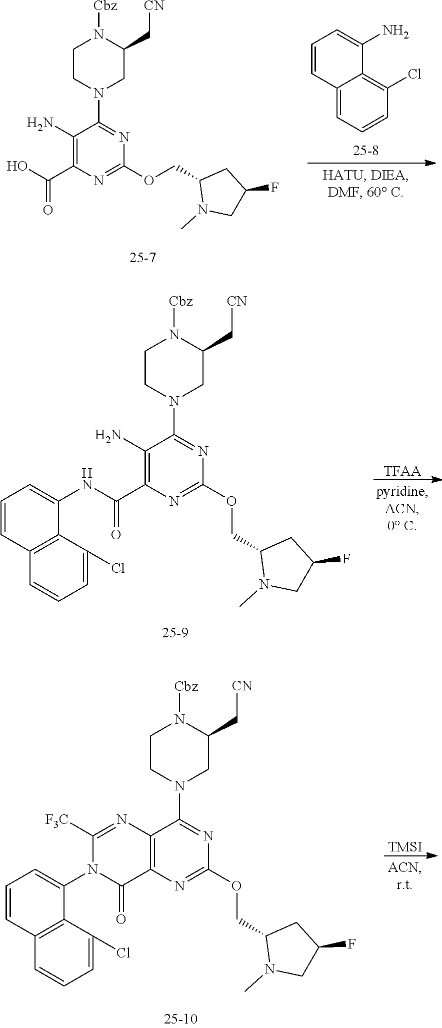

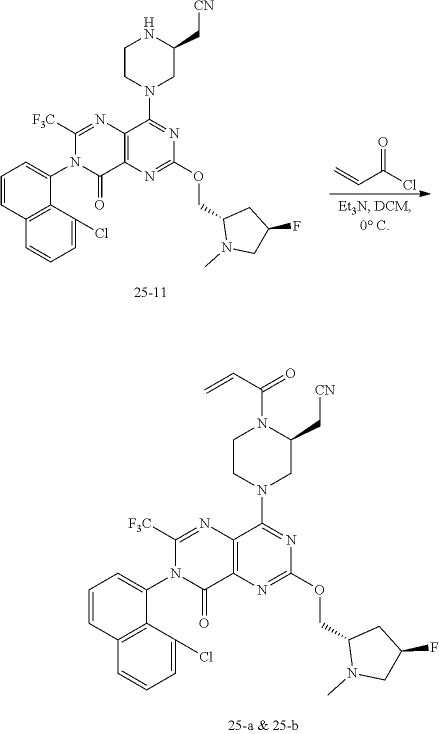

### Step 1: Synthesis of Compound 25-3

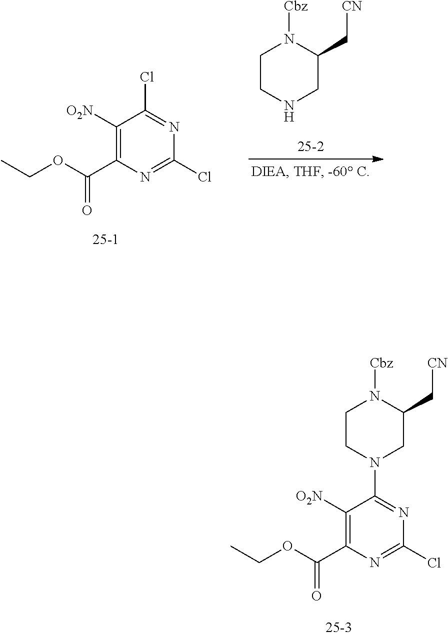 [SMILES: CCOC(=O)c1nc(Cl)nc(Cl)c1[N+](=O)[O-] ; N#CC[C@H]1CNCCN1C(=O)OCc1ccccc1 ; CCOC(=O)c1nc(Cl)nc(N2CCN(C(=O)OCc3ccccc3)[C@@H](CC#N)C2)c1[N+](=O)[O-]]

To a cooled (−60° C.) solution of ethyl 2,6-dichloro-5-nitropyrimidine-4-carboxylate (5.0 g, 0.019 mol, 1.0 eq.) in anhydrous THF (50 mL) was added a solution of benzyl (S′)-2-(cyanomethyl) piperazine-1-carboxylate (4.9 g, 0.019 mol, 1.0 eq.) and DIEA (3.6 g, 0.028 mol, 1.5 eq.) in anhydrous THF (40 mL). The reaction mixture was stirred at −60° C. under Ar for 1 h. LCMS showed starting material was consumed and desired product formed. The reaction mixture was concentrated and purified by silica column chromatography eluting with Pet.ether/EtOAc (3:1, v/v) to obtain ethyl (S)-6-(4-((benzyloxy)carbonyl)-3-(cyanomethyl) piperazin-1-yl)-2-chloro-5-nitropyrimidine-4-carboxylate (6.3 g, 69% yield).

LCMS (ESI, m/z): [M+1]⁺=489; RT=1.948 min.

### Step 2: Synthesis of Compound 25-4

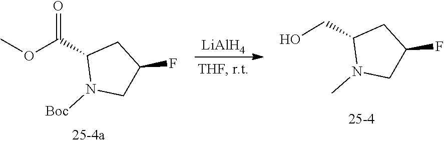

To a mixture of 1-(tert-butyl) 2-methyl (2S,4R)-4-fluoropyrrolidine-1,2-dicarboxylate (10.0 g, 0.040 mol, 1.0 eq.) in anhydrous THF (100 mL) was added LiAlH₄ (5.4 g, 0.142 mol, 3.5 eq.) in portions. The mixture was stirred at room temperature for 16 h. LCMS showed starting material was consumed and desired product formed. The reaction mixture was diluted with EtOAc (100 mL), and then quenched with H₂O (54 mL), 15% aq. NaOH (54 mL) and H₂O (162 mL). The resulting mixture was stirred vigorously at room temperature for 30 min and the precipitate was filtered off through celite. The filter cake was washed with EtOAc (20 mL×3). The organic filtrates were combined, washed with brine (100 mL), dried over anhydrous Na₂SO₄ and concentrated to obtain ((2S,4R)-4-fluoro-1-methylpyrrolidin-2-yl) methanol (4.06 g, 75% yield), which was used directly for the next step.

### Step 3: Synthesis of Compound 25-5

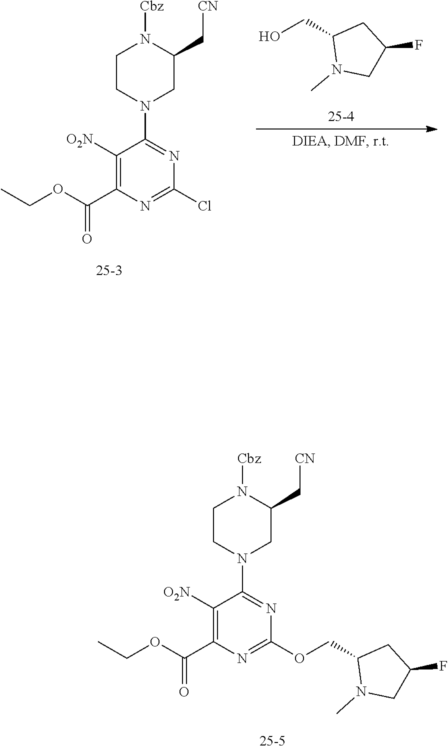

To a solution of ethyl (S)-6-(4-(tert-butoxycarbonyl)-2-methylpiperazin-1-yl)-2-chloro-5-nitropyrimidine-4-carboxylate (6.3 g, 0.013 mol, 1.0 eq.) and DIEA (3.3 g, 0.026 mol, 2.0 eq.) in anhydrous DMF (60 mL) was added ((2S,4R)-4-fluoro-1-methylpyrrolidin-2-yl) methanol (2.6 g, 0.019 mmol, 1.5 eq.). The mixture was stirred at room temperature for 16 h. LCMS showed starting material was consumed and desired product formed. The reaction mixture was diluted with H₂O (100 mL) and extracted with EtOAc (80 mL×3). The combined organic fractions were washed with brine (100 mL), dried over anhydrous Na₂SO₄ and concentrated. The residue was purified by silica column chromatography eluting with DCM/MeOH (15:1, v/v) to obtain ethyl 6-((S)-4-((benzyloxy)carbonyl)-3-(cyanomethyl) piperazin-1-yl)-2-(((2S,4R)-4-fluoro-1-methylpyrrolidin-2-yl) methoxy)-5-nitropyrimidine-4-carboxylate (4.4 g, 58% yield).

LCMS (ESI, m/z): [M+1]⁺=586; RT=1.092 min.

### Step 4: Synthesis of Compound 25-6

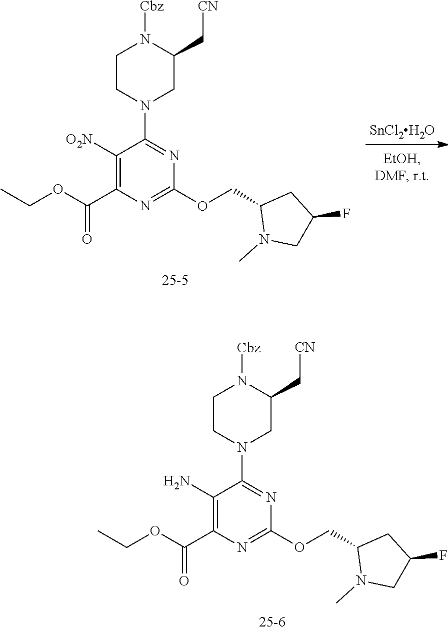 [SMILES: CCOC(=O)c1nc(OC[C@@H]2C[C@@H](F)CN2C)nc(N2CCN(C(=O)OCc3ccccc3)[C@@H](CC#N)C2)c1[N+](=O)[O-] ; "SnCl2·H2O" ; CCOC(=O)c1nc(OC[C@@H]2C[C@@H](F)CN2C)nc(N2CCN(C(=O)OCc3ccccc3)[C@@H](CC#N)C2)c1N]

To a solution of ethyl 6-((S)-4-((benzyloxy)carbonyl)-3-(cyanomethyl) piperazin-1-yl)-2-(((2S,4R)-4-fluoro-1-methylpyrrolidin-2-yl) methoxy)-5-nitropyrimidine-4-carboxylate (4.0 g, 0.07 mol, 1.0 eq.) in anhydrous DMF (20 mL)/EtOH (60 mL) was added SnCl₂·2H₂O (7.7 g, 0.34 mol, 5.0 eq.). The reaction mixture was stirred at room temperature under Ar for 16 h. LCMS showed that starting material was consumed and desired product formed. The reaction mixture was concentrated to remove EtOH and then diluted with EtOAc (120 mL), followed by the addition of aq. NaHCO₃ (sat. 180 mL). The resulting mixture was filtered through celite. The organic layer of the filtrate was separated and the aqueous layer was extracted with EtOAc (100 mL×2). The combined organic fractions were washed with brine (120 mL), dried over anhydrous Na₂SO₄ and concentrated. The residue was purified by silica column chromatography eluting with DCM/MeOH (15:1, v/v) to obtain ethyl 5-amino-6-((S)-4-((benzyloxy)carbonyl)-3-(cyanomethyl) piperazin-1-yl)-2-(((2S,4R)-4-fluoro-1-methylpyrrolidin-2-yl) methoxy) pyrimidine-4-carboxylate (2.4 g, 63% yield).

LCMS (ESI, m/z): [M+1]⁺=556; RT=1.025 min.

### Step 5: Synthesis of Compound 25-7

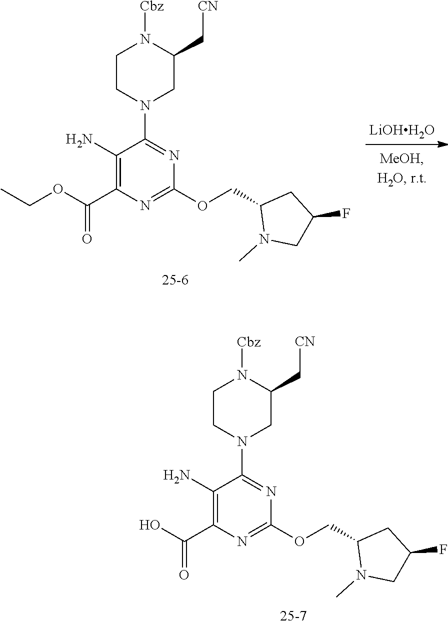 [SMILES: CCOC(=O)c1nc(OC[C@@H]2C[C@@H](F)CN2C)nc(N2CCN(C(=O)OCc3ccccc3)[C@@H](CC#N)C2)c1N ; "LiOH·H2O" ; CN1C[C@H](F)C[C@H]1COc1nc(C(=O)O)c(N)c(N2CCN(C(=O)OCc3ccccc3)[C@@H](CC#N)C2)n1]

To a solution of ethyl 5-amino-6-((S)-4-((benzyloxy)carbonyl)-3-(cyanomethyl) piperazin-1-yl)-2-(((2S,4R)-4-fluoro-1-methylpyrrolidin-2-yl) methoxy) pyrimidine-4-carboxylate (2.4 g, 0.004 mol, 1.0 eq.) in MeOH (60 mL) and H₂O (10 mL) was added LiOH·H₂O (0.91 g, 0.022 mol, 5.0 eq.). The mixture was stirred at room temperature for 2 h. LCMS showed starting material was consumed and desired product formed. The reaction mixture was acidified with aq. HCl (1.0 M) to adjust pH=6, and then concentrated to dryness to concentrated to obtain 5-amino-6-((S)-4-((benzyloxy)carbonyl)-3-(cyanomethyl) piperazin-1-yl)-2-(((2S,4R)-4-fluoro-1-methylpyrrolidin-2-yl) methoxy) pyrimidine-4-carboxylic acid (3.9 g, crude), which was used directly for the next step

LCMS (ESI, m/z): [M+1]⁺=528; RT=1.120 min.

### Step 6: Synthesis of Compound 25-9

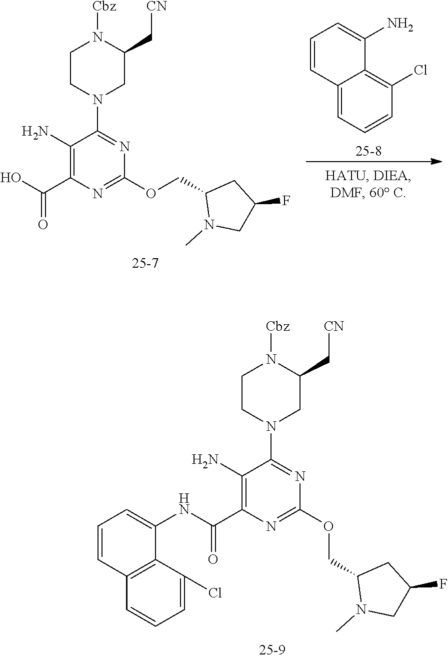

To a solution of 5-amino-6-((S)-4-((benzyloxy)carbonyl)-3-(cyanomethyl) piperazin-1-yl)-2-(((2S,4R)-4-fluoro-1-methylpyrrolidin-2-yl) methoxy) pyrimidine-4-carboxylic acid (3.9 g, 7.0 mmol, 1.0 eq.) and 8-methylnaphthalen-1-amine (0.9 g, 5.0 mmol, 0.7 eq.) in anhydrous DMF (10 mL) was added DIEA (2.9 g, 20 mmol, 3.0 eq.), followed by the addition of HATU (3.1 g, 8.0 mmol, 1.1 eq.). The reaction mixture was stirred at 60° C. under Ar for 1 h. LCMS showed starting material was consumed and desired product formed. The reaction mixture was cooled to room temperature, diluted with water (80 mL) and extracted with EtOAc (50 mL×3). The combined organic fractions were washed with brine (80 mL), dried over anhydrous Na₂SO₄ and concentrated. The residue was purified by silica column chromatography eluting with DCM/MeOH (15:1, v/v) to obtain benzyl (S)-4-(5-amino-6-((8-chloronaphthalen-1-yl) carbamoyl)-2-(((2S,4R)-4-fluoro-1-methylpyrrolidin-2-yl) methoxy) pyrimidin-4-yl)-2-(cyanomethyl) piperazine-1-carboxylate (2.0 g, 59% yield).

LCMS (ESI, m/z): [M+1]⁺=687; RT=1.222 min.

### Step 7: Synthesis of Compound 25-10

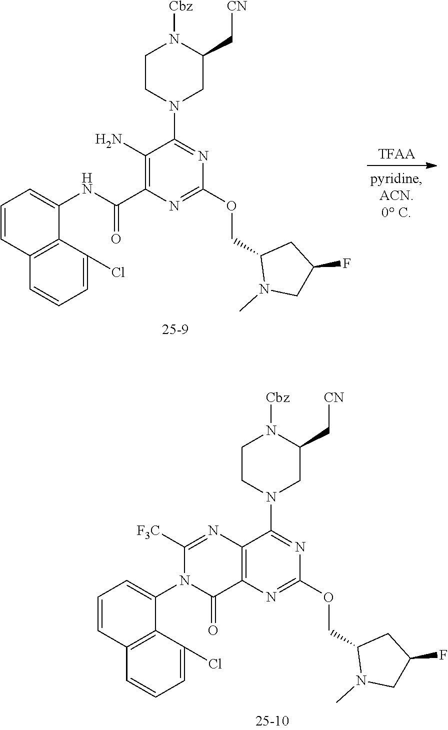 [SMILES: CN1C[C@H](F)C[C@H]1COc1nc(C(=O)Nc2cccc3cccc(Cl)c23)c(N)c(N2CCN(C(=O)OCc3ccccc3)[C@@H](CC#N)C2)n1 ; "TFAA" ; CN1C[C@H](F)C[C@H]1COc1nc(N2CCN(C(=O)OCc3ccccc3)[C@@H](CC#N)C2)c2nc(C(F)(F)F)n(-c3cccc4cccc(Cl)c34)c(=O)c2n1]

To a cooled (0° C.) solution of benzyl (S)-4-(5-amino-6-((8-chloronaphthalen-1-yl) carbamoyl)-2-(((2S,4R)-4-fluoro-1-methylpyrrolidin-2-yl) methoxy) pyrimidin-4-yl)-2-(cyanomethyl) piperazine-1-carboxylate (1.00 g, 1.46 mmol, 1.0 eq.) in anhydrous ACN (3.0 mL) was added pyridine (1.15 g, 14.6 mmol, 10.0 eq.), followed by the addition of TFAA (1.84 g, 8.75 mmol, 6.0 eq.). The mixture was stirred at 0° C. for 0.5 h. LCMS showed starting material was consumed and desired product formed. The reaction mixture was quenched with aq. NH₄Cl (sat. 40 mL) and extracted with EtOAc (20 mL×3). The combined organic fractions were dried over anhydrous Na₂SO₄ and concentrated. The residue was purified by prep-TLC eluting with DCM/MeOH (10:1, v/v) to obtain benzyl (S)-4-(7-(8-chloronaphthalen-1-yl)-2-(((2S,4R)-4-fluoro-1-methylpyrrolidin-2-yl) methoxy)-8-oxo-6-(trifluoromethyl)-7,8-dihydropyrimido[5,4-d] pyrimidin-4-yl)-2-(cyanomethyl) piperazine-1-carboxylate (120 mg, 11% yield).

LCMS (ESI, m/z): [M+1]⁺=764; RT=1.371 min.

### Step 8: Synthesis of Compound 25-11

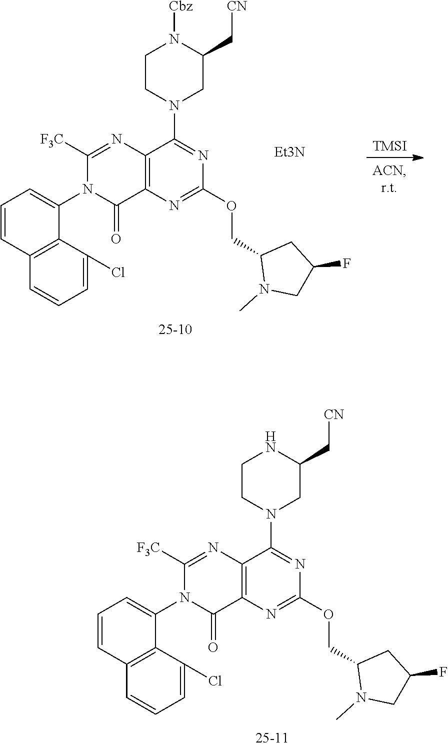 [SMILES: "Et3N" ; "TMSI" ; CN1C[C@H](F)C[C@H]1COc1nc(N2CCN(C(=O)OCc3ccccc3)[C@@H](CC#N)C2)c2nc(C(F)(F)F)n(-c3cccc4cccc(Cl)c34)c(=O)c2n1 ; CN1C[C@H](F)C[C@H]1COc1nc(N2CCN[C@@H](CC#N)C2)c2nc(C(F)(F)F)n(-c3cccc4cccc(Cl)c34)c(=O)c2n1]

To a solution of benzyl (S)-4-(7-(8-chloronaphthalen-1-yl)-2-(((2S,4R)-4-fluoro-1-methylpyrrolidin-2-yl) methoxy)-8-oxo-6-(trifluoromethyl)-7,8-dihydropyrimido[5,4-d] pyrimidin-4-yl)-2-(cyanomethyl) piperazine-1-carboxylate (100 mg, 0.131 mmol) in anhydrous ACN (5.0 mL) was added TMSI (262 mg, 1.31 mmol), and the mixture was stirred at room temperature for 1 h. LCMS showed starting material was consumed and desired product formed. The reaction mixture was treated with Et₃N (1.0 mL) and concentrated and purified by prep-TLC eluting with DCM/MeOH (10:1, v/v) to obtain 2-((S)-4-(7-(8-chloronaphthalen-1-yl)-2-(((2S,4R)-4-fluoro-1-methylpyrrolidin-2-yl) methoxy)-8-oxo-6-(trifluoromethyl)-7,8-dihydropyrimido[5,4-d] pyrimidin-4-yl) piperazin-2-yl) acetonitrile (10 mg, 85% yield).

LCMS (ESI, m/z): [M+1]⁺=631; RT=0.870 min.

### Step 9: Synthesis of Compounds 25-a & 25-b

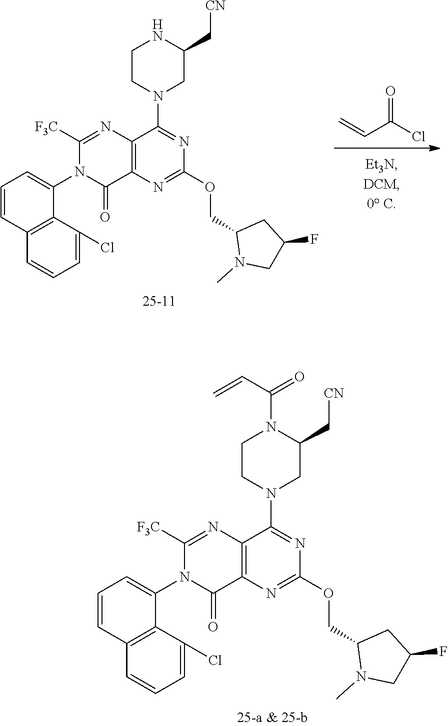

To a cooled (0° C.) solution of 2-((S)-4-(7-(8-chloronaphthalen-1-yl)-2-(((2S,4R)-4-fluoro-1-methylpyrrolidin-2-yl) methoxy)-8-oxo-6-(trifluoromethyl)-7,8-dihydropyrimido[5,4-d] pyrimidin-4-yl) piperazin-2-yl) acetonitrile (70 mg, 0.10 mmol, 1.0 eq.) and Et₃N (31 mg, 0.31 mmol, 3.0 eq.) in DCM (2.5 mL) was added dropwise a solution of acryloyl chloride (13.8 mg, 0.14 mmol, 1.5 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×2). The combined organic fractions were dried over anhydrous Na₂SO₄ and concentrated. The residue was purified by prep-HPLC (ACN-H₂O+0.1% HCOOH) and then SFC to obtain 2-((S)-1-acryloyl-4-(7-(8-chloronaphthalen-1-yl)-2-(((2S,4R)-4-fluoro-1-methylpyrrolidin-2-yl) (3.2 mg, 4% yield, 25-a) and 2-((S)-1-acryloyl-4-(7-(8-chloronaphthalen-1-yl)-2-(((2S,4R)-4-fluoro-1-methylpyrrolidin-2-yl) (3.5 mg, 4% yield, 25-b).

25-a:

LCMS (ESI, m/z): [M+1]⁺=685; RT=1.178 min;

¹H NMR (400 MHz, CDCl₃) δ 8.08 (d, J=7.4 Hz, 1H), 7.91 (d, J=6.7 Hz, 1H), 7.67-7.55 (m, 2H), 7.52-7.41 (m, 2H), 6.72-6.53 (m, 1H), 6.41 (d, J=16.6 Hz, 1H), 5.84 (d, J=10.0 Hz, 1H), 5.70-4.77 (m, 3H), 4.64 (s, 2H), 4.04 (s, 1H), 3.86-3.45 (m, 2H), 3.26 (s, 1H), 2.98-2.87 (m, 1H), 2.87-2.71 (m, 2H), 2.67 (s, 3H), 2.41-2.09 (m, 3H), 1.39-1.27 (m, 2H);

¹⁹F NMR (400 MHz, CDCl₃) δ −64.79, −170.75.

25-b:

LCMS (ESI, m/z): [M+1]⁺=685; RT=1.704 min;

¹H NMR (400 MHz, CDCl₃) δ 8.09 (d, J=8.6 Hz, 1H), 7.91 (d, J=8.1 Hz, 1H), 7.66-7.56 (m, 2H), 7.49-7.42 (m, 2H), 6.71-6.53 (m, 1H), 6.42 (d, J=16.3 Hz, 1H), 5.85 (d, J=10.5 Hz, 1H), 5.56-4.84 (m, 3H), 4.70-4.45 (m, 2H), 4.13-3.82 (m, 1H), 3.76-3.44 (m, 2H), 3.20 (s, 1H), 2.89 (s, 1H), 2.86-2.62 (m, 2H), 2.61 (s, 3H), 2.40-2.11 (m, 3H), 1.39-1.26 (m, 2H);

¹⁹F NMR (400 MHz, CDCl₃) δ −64.83, −170.74.

### Example 26

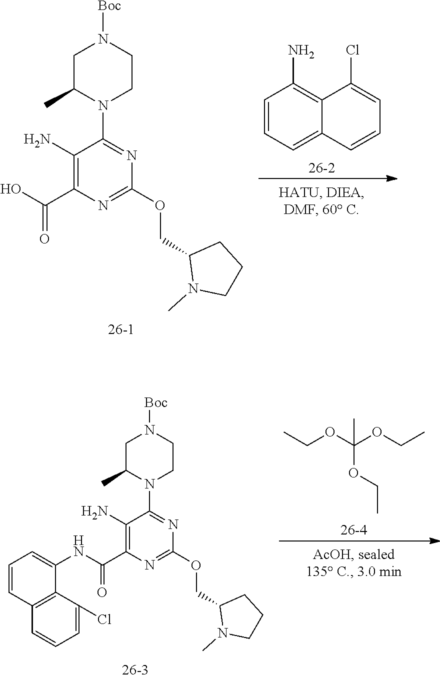

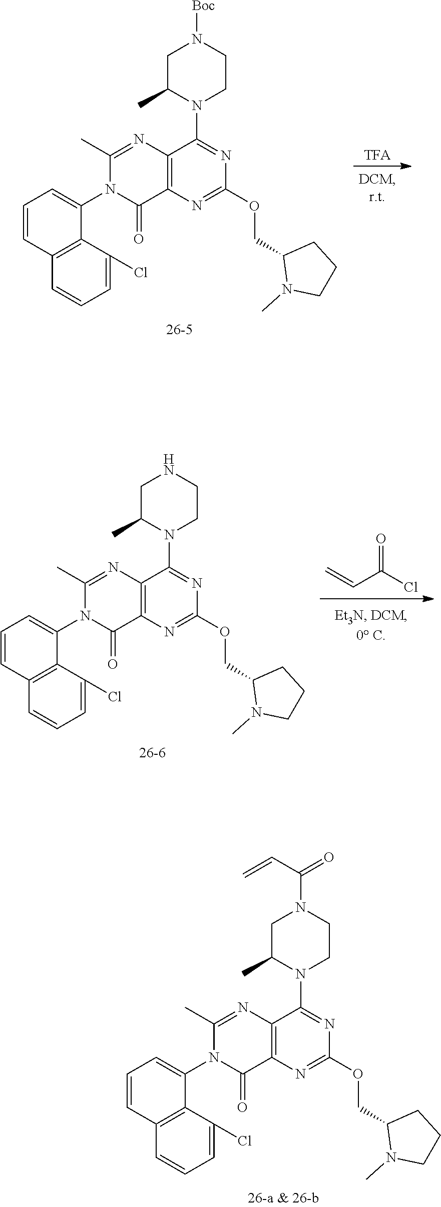

### Step 1: Synthesis of Compound 26-3

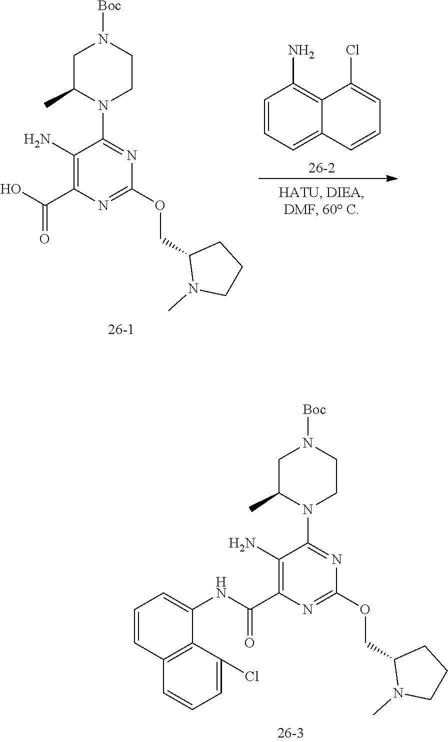

To a solution of 5-amino-6-((S)-4-(tert-butoxycarbonyl)-2-methylpiperazin-1-yl)-2-(((S)-1-methylpyrrolidin-2-yl) methoxy) pyrimidine-4-carboxylic acid (1.80 g, 4.0 mmol, 1.0 eq.) and 8-chloronaphthalen-1-amine (0.49 g, 3.0 mmol, 0.7 eq.) in anhydrous DMF (20 mL), was added DIEA (1.55 g, 12.0 mmol, 3.0 eq.), followed by the addition of HATU (1.52 g, 4.0 mmol, 1.0 eq.). The mixture was stirred at 60° C. under Ar for 1 h. LCMS showed starting material was consumed and desired product formed. The reaction mixture was cooled to room temperature, diluted with water (100 mL) and extracted with EtOAc (60 mL×3). The combined organic fractions were washed with brine (100 mL), dried over anhydrous Na₂SO₄ and concentrated. The residue was purified by silica column chromatography eluting with DCM/MeOH (10:1, v/v) to obtain tert-butyl (S)-4-(5-amino-6-((8-chloronaphthalen-1-yl) carbamoyl)-2-(((S)-1-methylpyrrolidin-2-yl) methoxy) pyrimidin-4-yl)-3-methylpiperazine-1-carboxylate (1.06 g, 63% yield, 26-3).

LCMS (ESI, m/z): [M+1]⁺=610; RT=1.259 min.

### Step 2: Synthesis of Compound 26-5

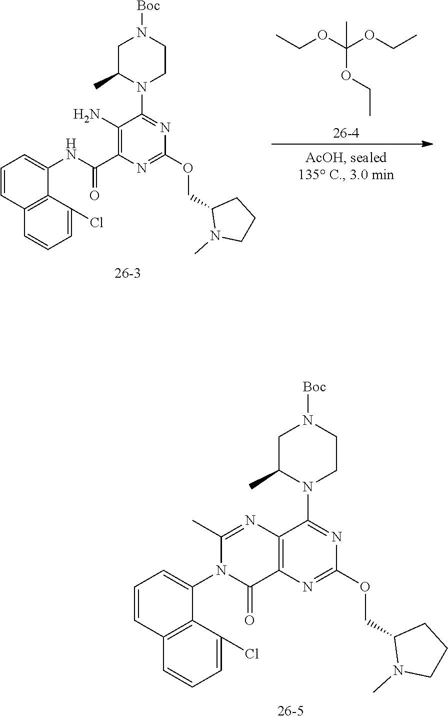

To a mixture of tert-butyl (S)-4-(5-amino-6-((8-chloronaphthalen-1-yl) carbamoyl)-2-(((S)-1-methylpyrrolidin-2-yl) methoxy) pyrimidin-4-yl)-3-methylpiperazine-1-carboxylate (500 mg, 0.82 mmol, 1.0 eq.) and AcOH (5.0 mL) was added 1,1,1-triethoxyethane (2.25 mL, 12.3 mmol, 15.0 eq.). The mixture was stirred at 135° C. in a sealed tube for 3 min. LCMS showed starting material was consumed and desired product formed. The reaction mixture was cooled, quenched with aq. NaHCO₃ (120 mL) and extracted with DCM (60 mL×3). The combined organic fractions were dried over anhydrous Na₂SO₄ and concentrated. The residue was purified by prep-TLC eluting with DCM/MeOH (10:1, v/v) to obtain tert-butyl (S)-4-(7-(8-chloronaphthalen-1-yl)-6-methyl-2-(((S)-1-methylpyrrolidin-2-yl) methoxy)-8-oxo-7,8-dihydropyrimido[5,4-d] pyrimidin-4-yl)-3-methylpiperazine-1-carboxylate (180 mg, 35% yield, 26-5).

LCMS (ESI, m/z): [M+1]⁺=634; RT=1.174 min.

### Step 3: Synthesis of Compound 26-6

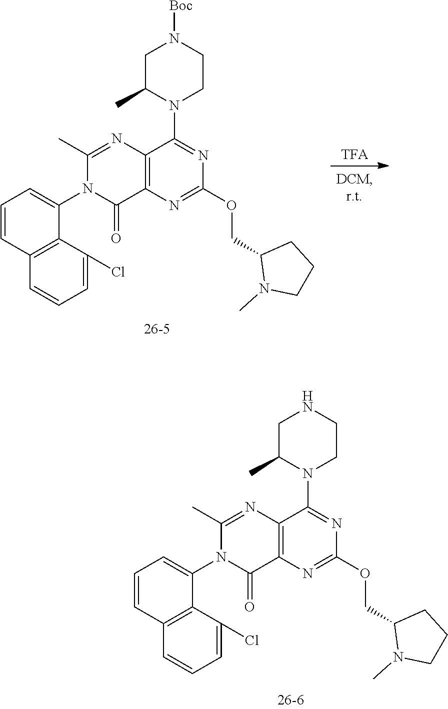 [SMILES: Cc1nc2c(N3CCN(C(=O)OC(C)(C)C)C[C@@H]3C)nc(OC[C@@H]3CCCN3C)nc2c(=O)n1-c1cccc2cccc(Cl)c12 ; Cc1nc2c(N3CCNC[C@@H]3C)nc(OC[C@@H]3CCCN3C)nc2c(=O)n1-c1cccc2cccc(Cl)c12 ; "TFA"]

To a solution of tert-butyl (S)-4-(7-(8-chloronaphthalen-1-yl)-6-methyl-2-(((S)-1-methylpyrrolidin-2-yl) methoxy)-8-oxo-7,8-dihydropyrimido[5,4-d]pyrimidin-4-yl)-3-methylpiperazine-1-carboxylate (180 mg, 0.28 mmol) in DCM (5.0 mL) was TFA (2.0 mL), and the mixture was stirred at room temperature for 1 h. LCMS showed starting material was consumed and desired product formed. The reaction mixture was concentrated and the residue was treated with aq. NaHCO₃ (sat. 20 mL). The resulting mixture was extracted with DCM (10 mL×3). The combined organic fractions were dried over anhydrous Na₂SO₄ and concentrated to obtain 3-(8-chloronaphthalen-1-yl)-2-methyl-8-((S)-2-methylpiperazin-1-yl)-6-(((S)-1-methylpyrrolidin-2-yl) methoxy) pyrimido[5,4-d] pyrimidin-4(3H)-one (136 mg, 89% yield, 26-6), which was used directly for the next step.

LCMS (ESI, m/z): [M+1]⁺=534; RT=0.758 min.

### Step 4: Synthesis of Compounds 26-a and 26-b

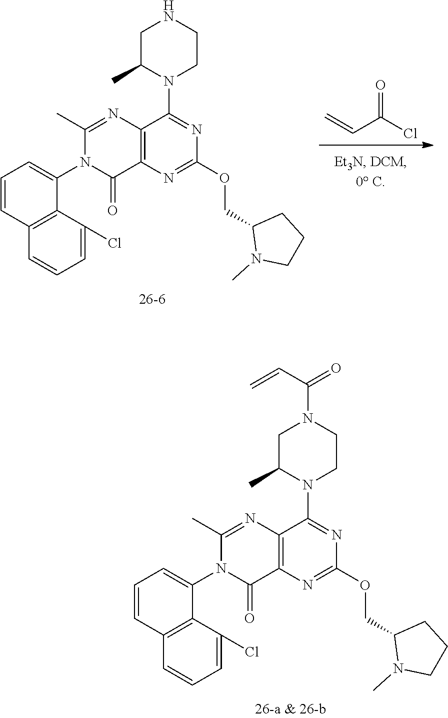

To a cooled (0° C.) solution of 3-(8-chloronaphthalen-1-yl)-2-methyl-8-((S)-2-methylpiperazin-1-yl)-6-(((S)-1-methylpyrrolidin-2-yl) methoxy) pyrimido[5,4-d]pyrimidin-4(3H)-one (130 mg, 0.24 mmol, 1.0 eq.) and Et₃N (74 mg, 0.73 mmol, 3.0 eq.) in anhydrous DCM (5 mL) was added dropwise a solution of acryloyl chloride (28 mg, 0.43 mmol, 1.3 eq.) in anhydrous DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((S)-4-acryloyl-2-methylpiperazin-1-yl)-3-(8-chloronaphthalen-1-yl)-2-methyl-6-(((S)-1-methylpyrrolidin-2-yl) methoxy) pyrimido[5,4-d] pyrimidin-4(3H)-one (22 mg, 15% yield, 26-a), and 8-((S)-4-acryloyl-2-methylpiperazin-1-yl)-3-(8-chloronaphthalen-1-yl)-2-methyl-6-(((S)-1-methylpyrrolidin-2-yl) methoxy) pyrimido[5,4-d] pyrimidin-4(3H)-one (20 mg, 15% yield, 26-b).

26-a:

LCMS (ESI, m/z): [M+1]⁺=588; RT=1.745 min;

¹H NMR (400 MHz, CDCl₃) δ 8.05 (d, J=7.4 Hz, 1H), 7.91 (d, J=7.3 Hz, 1H), 7.67-7.61 (m, 1H), 7.58 (dd, J=7.5, 1.1 Hz, 1H), 7.48-7.42 (m, 1H), 7.39 (d, J=7.1 Hz, 1H), 6.71-6.53 (m, 1H), 6.44-6.34 (m, 1H), 5.77 (d, J=10.7 Hz, 1H), 5.34-4.27 (m, 3H), 4.12-3.70 (m, 1H), 3.67-3.48 (m, 2H), 3.43-2.82 (m, 3H), 2.59 (s, 3H), 2.46-2.27 (m, 1H), 2.12 (s, 3H), 2.11-2.01 (m, 1H), 2.00-1.69 (m, 4H), 1.40-1.35 (m, 3H).

26-b:

LCMS (ESI, m/z): [M+1]⁺=588; RT=1.752 min;

¹H NMR (400 MHz, CDCl₃) δ 8.05 (d, J=8.3 Hz, 1H), 7.91 (d, J=8.1 Hz, 1H), 7.64 (t, J=7.8 Hz, 1H), 7.58 (d, J=7.5 Hz, 1H), 7.45 (t, J=7.8 Hz, 1H), 7.40 (d, J=7.2 Hz, 1H), 6.69-6.53 (m, 1H), 6.38 (d, J=16.7 Hz, 1H), 5.77 (d, J=10.2 Hz, 1H), 5.35-4.32 (m, 3H), 4.13-3.73 (m, 1H), 3.67-3.47 (m, 2H), 3.40-3.06 (m, 2H), 2.92 (s, 1H), 2.57 (s, 3H), 2.45-2.37 (m, 1H), 2.29-2.21 (m, 1H), 2.13 (s, 3H), 2.10-2.05 (m, 1H), 1.86-1.81 (m, 4H). 1.38 (d, J=8.0 Hz, 3H).

### Example 27

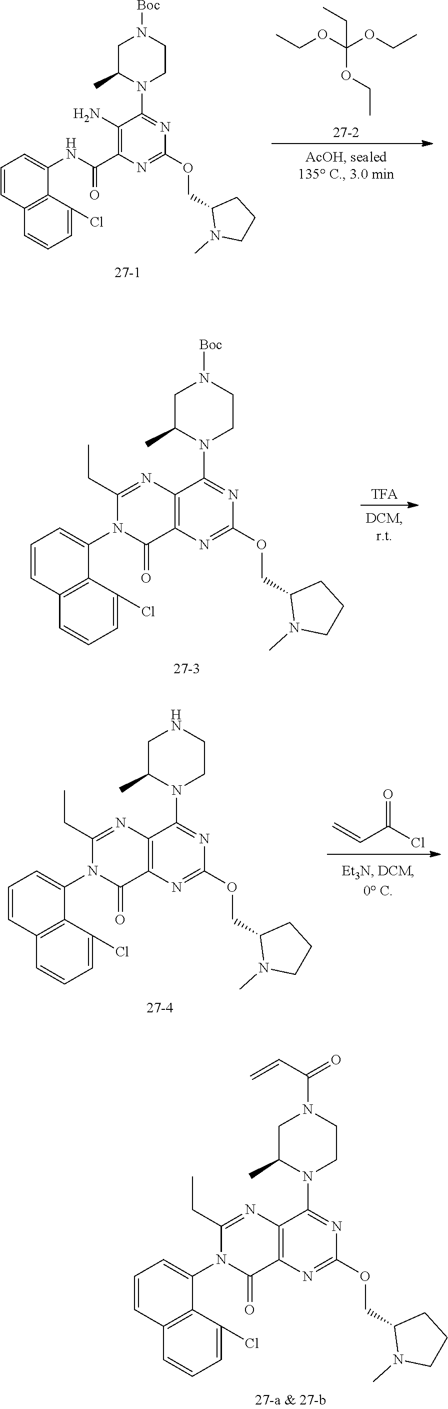

### Step 1: Synthesis of Compound 27-3

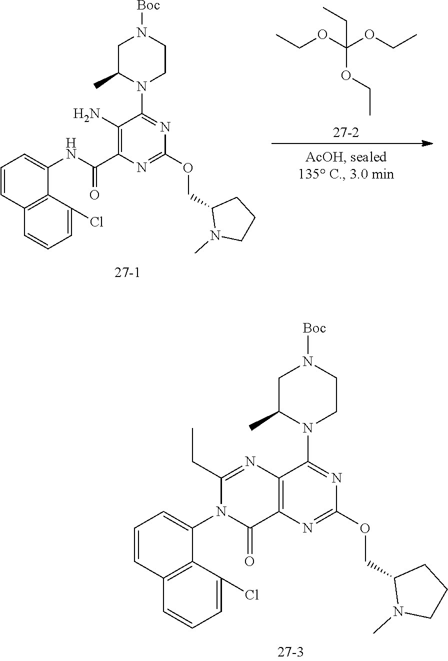

To a mixture of tert-butyl (S)-4-(5-amino-6-((8-chloronaphthalen-1-yl) carbamoyl)-2-(((S′)-1-methylpyrrolidin-2-yl) methoxy) pyrimidin-4-yl)-3-methylpiperazine-1-carboxylate (500 mg, 0.82 mmol, 1.0 eq.) and AcOH (5.0 mL) was added 1,1,1-tri ethoxypropane (2.5 mL, 12.3 mmol, 15.0 eq.). The mixture was stirred at 135° C. in a sealed tube for 3 min. LCMS showed starting material was consumed and desired product formed. The reaction mixture was cooled, quenched with aq. NaHCO₃ (120 mL) and extracted with DCM (60 mL×3). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(7-(8-fluoronaphthalen-1-yl)-2-(((S)-1-methylpyrrolidin-2-yl) methoxy)-8-oxo-6-(trifluoromethyl)-7,8-dihydro pyrimido[5,4-d] pyrimidin-4-yl) piperazine-1-carboxylate (120 mg, 41% yield, 27-3).

LCMS (ESI, m/z): [M+1]⁺=648; RT=1.218 min.

### Step 2: Synthesis of Compound 27-4

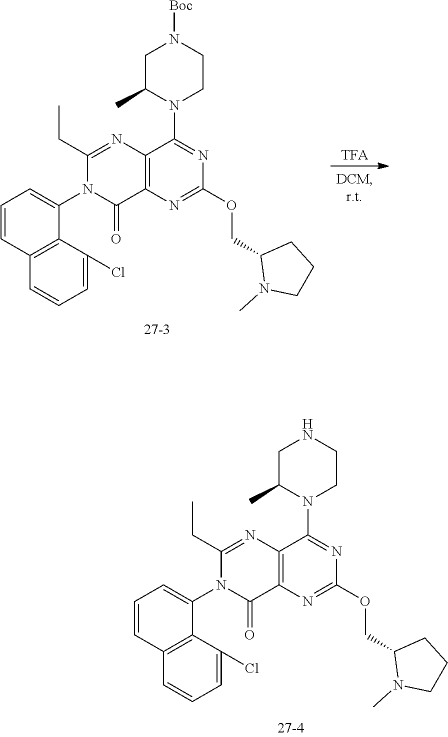 [SMILES: CCc1nc2c(N3CCN(C(=O)OC(C)(C)C)C[C@@H]3C)nc(OC[C@@H]3CCCN3C)nc2c(=O)n1-c1cccc2cccc(Cl)c12 ; CCc1nc2c(N3CCNC[C@@H]3C)nc(OC[C@@H]3CCCN3C)nc2c(=O)n1-c1cccc2cccc(Cl)c12 ; "TFA"]

To a solution of benzyl (S)-2-(cyanomethyl)-4-(7-(8-fluoronaphthalen-1-yl)-2-(((S)-1-methylpyrrolidin-2-yl) methoxy)-8-oxo-6-(trifluoromethyl)-7,8-dihydropyrimido[5,4-d] pyrimidin-4-yl) piperazine-1-carboxylate (220 mg, 0.34 mmol) in DCM (5.0 mL) was added TFA (2.0 mL), and the mixture was stirred at room temperature for 1 h. LCMS showed starting material was consumed and desired product formed. The reaction mixture was concentrated and the residue was treated with aq.NaHCO₃ (sat. 20 mL). The resulting mixture was extracted with DCM (10 mL×3). The combined organic fractions were dried over anhydrous Na₂SO₄ and concentrated to obtain 3-(8-chloronaphthalen-1-yl)-2-ethyl-8-((S)-2-methylpiperazin-1-yl)-6-(((S)-1-methylpyrrolidin-2-yl) methoxy) pyrimido [5,4-d]pyrimidin-4(3H)-one (170 mg, 90% yield, 27-4), which was used directly for the next step.

LCMS (ESI, m/z): [M+1]⁺=548; RT=0.720 min.

### Step 3: Synthesis of Compounds 27-a & 27-b

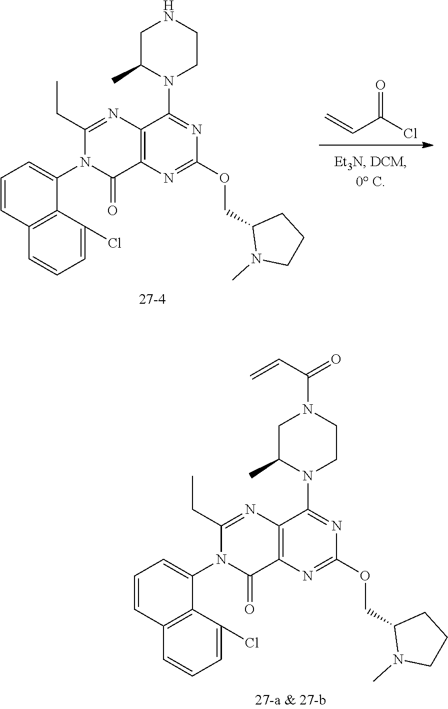

To a cooled (0° C.) solution of 3-(8-chloronaphthalen-1-yl)-2-ethyl-8-((S)-2-methylpiperazin-1-yl)-6-(((S)-1-methylpyrrolidin-2-yl) methoxy) pyrimido[5,4-d]pyrimidin-4(3H)-one (170 mg, 0.31 mmol, 1.0 eq.) and Et₃N (94 mg, 0.93 mmol, 3.0 eq.) in anhydrous DCM (5 mL) was added dropwise a solution of acryloyl chloride (42 mg, 0.47 mmol, 1.5 eq.) in anhydrous DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((S)-4-acryloyl-2-methylpiperazin-1-yl)-3-(8-chloronaphthalen-1-yl)-2-ethyl-6-(((S)-1-methylpyrrolidin-2-yl) methoxy) pyrimido[5,4-d] pyrimidin-4(3H)-one (32 mg, 17% yield, 27-a), and 8-((S)-4-acryloyl-2-methylpiperazin-1-yl)-3-(8-chloronaphthalen-1-yl)-2-ethyl-6-(((S)-1-methylpyrrolidin-2-yl) methoxy) pyrimido[5,4-d] pyrimidin-4(3H)-one (25 mg, 13% yield, 27-b).

27-a:

LCMS (ESI, m/z): [M+1]⁺=602; RT=1.840 min;

¹H NMR (400 MHz, CDCl₃) δ 8.03 (d, J=8.1 Hz, 1H), 7.89 (d, J=8.1 Hz, 1H), 7.62 (t, J=7.7 Hz, 1H), 7.55 (d, J=7.3 Hz, 1H), 7.43 (t, J=7.8 Hz, 1H), 7.36 (d, J=6.9 Hz, 1H), 6.73-6.49 (m, 1H), 6.37 (d, J=16.7 Hz, 1H), 5.76 (d, J=9.8 Hz, 1H), 5.40-4.28 (m, 3H), 4.13-3.70 (m, 1H), 3.67-3.44 (m, 2H), 3.38-3.03 (m, 2H), 2.87 (s, 1H), 2.56 (s, 3H), 2.44-2.16 (m, 5H), 2.13-2.02 (m, 1H), 1.90-1.74 (m, 3H), 1.43-1.33 (m, 3H), 1.16 (t, J=6.9 Hz, 3H).

27-b:

CMS (ESI, m/z): [M+1]⁺=602; RT=1.847 min;

¹H NMR (400 MHz, CDCl₃) δ 8.03 (d, J=8.3 Hz, 1H), 7.89 (d, J=7.6 Hz, 1H), 7.65-7.59 (m, 1H), 7.55 (dd, J=7.4, 0.9 Hz, 1H), 7.43 (t, J=7.9 Hz, 1H), 7.36 (d, J=7.2 Hz, 1H), 6.73-6.50 (m, 1H), 6.37 (d, J=16.7 Hz, 1H), 5.76 (d, J=10.3 Hz, 1H), 5.51-4.34 (m, 3H), 4.09-3.77 (m, 1H), 3.68-3.42 (m, 2H), 3.37-3.05 (m, 2H), 2.90 (s, 1H), 2.56 (s, 3H), 2.46-2.13 (m, 5H), 2.12-2.02 (m, 1H), 1.92-1.74 (m, 3H), 1.38 (d, J=6.7 Hz, 3H), 1.15 (t, J=7.2 Hz, 3H).

### Example 28

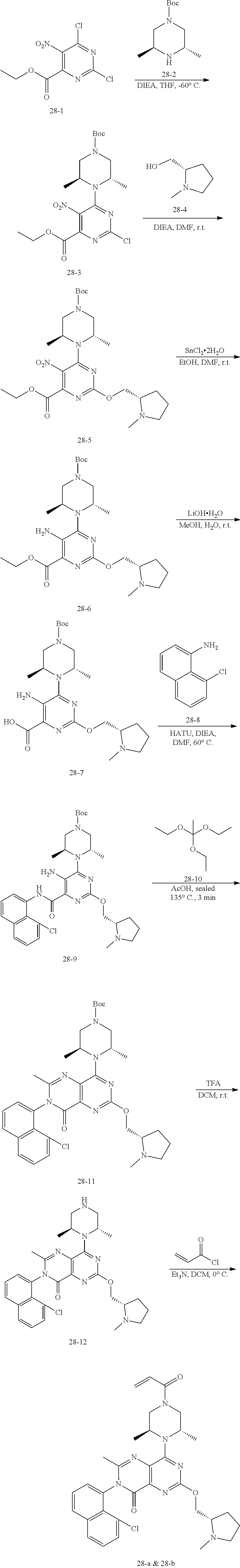

### Step 1: Synthesis of Compound 28-3

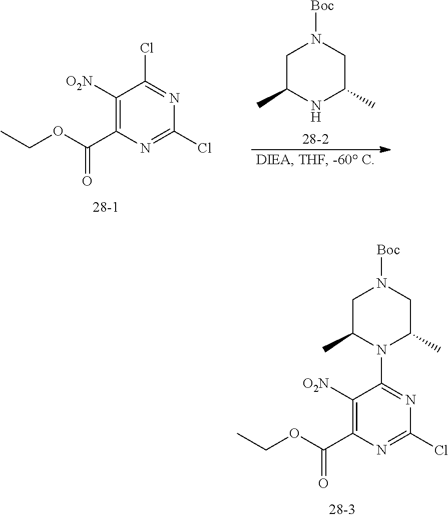

To a cooled (−60° C.) solution of ethyl 2,6-dichloro-5-nitropyrimidine-4-carboxylate (6.21 g, 23.4 mmol, 1.0 eq.) in anhydrous THF (40 mL) was added dropwise a solution of tert-butyl (3S,5S)-3,5-dimethylpiperazine-1-carboxylate (5.00 g, 23.4 mmol, 1.0 eq.) and DIEA (4.52 g, 35.0 mmol, 1.5 eq.) in anhydrous THF (30 mL). The mixture was stirred at −60° C. for 1 h. TLC showed the reaction was completed. The mixture was concentrated in vacuo and the residue was purified by silica column chromatography eluting with Pet.ether/EtOAc (2:1, v/v) to obtain ethyl 6-((2S,6S)-4-(tert-butoxycarbonyl)-2,6-dimethylpiperazin-1-yl)-2-chloro-5-nitropyrimidine-4-carboxylate (8.80 g, 85% yield, 28-3)

### Step 2: Synthesis of Compound 28-5

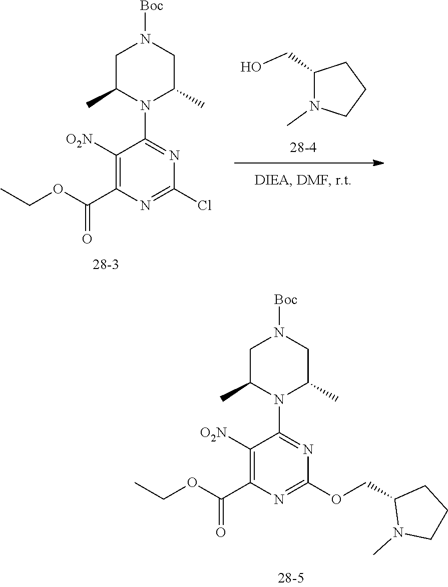

To a solution of ethyl 6-((2S,6S)-4-(tert-butoxycarbonyl)-2,6-dimethylpiperazin-1-yl)-2-chloro-5-nitropyrimidine-4-carboxylate (8.80 g, 19.7 mmol, 1.0 eq.) and DIEA (5.13 g, 39.7 mmol, 2.0 eq.) in anhydrous DMF (30 mL) was added (S)-(1-methylpyrrolidin-2-yl)methanol (3.43 g, 29.8 mmol, 1.5 eq). The mixture was stirred at room temperature for 3 h. LCMS showed starting material was consumed and desired product formed. The solution was diluted with brine (120 mL) and extracted with EtOAc (80 mL×2). The combined organic layers were dried over anhydrous Na₂SO₄ and concentrated. The residue was purified by silica column chromatography eluting with DCM/MeOH (15:1, v/v) to obtain ethyl 6-((2S,6S)-4-(tert-butoxycarbonyl)-2,6-dimethylpiperazin-1-yl)-2-(((S)-1-methylpyrrolidin-2-yl)methoxy)-5-nitropyrimidine-4-carboxylate (10.00 g, 96% yield, 28-5).

LCMS (ESI, m/z): [M+1]⁺=523; RT=1.125 min.

### Step 3: Synthesis of Compound 28-6

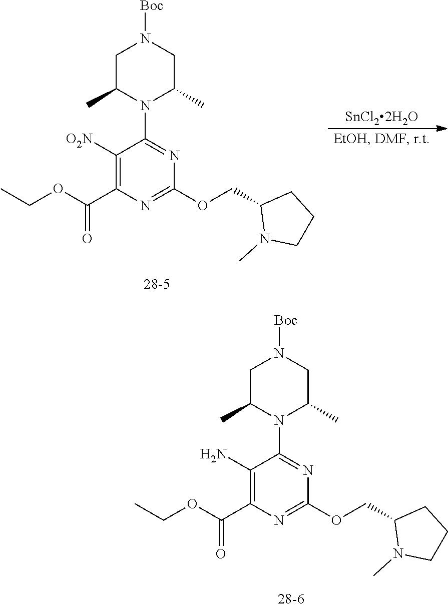 [SMILES: CCOC(=O)c1nc(OC[C@@H]2CCCN2C)nc(N2[C@@H](C)CN(C(=O)OC(C)(C)C)C[C@@H]2C)c1[N+](=O)[O-] ; "EtOH, DMF, r.t." ; "SnCl2·2H2O" ; CCOC(=O)c1nc(OC[C@@H]2CCCN2C)nc(N2[C@@H](C)CN(C(=O)OC(C)(C)C)C[C@@H]2C)c1N]

To a solution of ethyl 6-((2S,6S)-4-(tert-butoxycarbonyl)-2,6-dimethylpiperazin-1-yl)-2-(((S)-1-methylpyrrolidin-2-yl)methoxy)-5-nitropyrimidine-4-carboxylate (10.00 g, 19.2 mmol, 1.0 eq.) in anhydrous EtOH (50 mL)/DMF (50 mL) was added SnCl₂·2H₂O (21.66 g, 96.0 mmol, 5.0 eq.). The solution was stirred at room temperature for 3 hours. LCMS showed starting material was consumed and desired product formed. The reaction mixture was concentrated to remove EtOH and then diluted with EtOAc (150 mL), followed by the addition of aq. NaHCO₃ (sat. 200 mL). The resulting mixture was filtered through celite. The organic layer of the filtrate was separated and the aqueous layer was extracted with EtOAc (120 mL×2). The combined organic fractions were washed with brine (200 mL), dried over anhydrous Na₂SO₄ and concentrated. The residue was purified by silica column chromatography eluting with DCM/MeOH (10:1, v/v) to obtain ethyl 5-amino-6-((2S,6S)-4-(tert-butoxycarbonyl)-2,6-dimethylpiperazin-1-yl)-2-(((S)-1-methylpyrrolidin-2-yl)methoxy)pyrimidine-4-carboxylate (2.00 g, 21% yield, 28-6).

LCMS (ESI, m/z): [M+1]⁺=451; RT=0.928 min.

### Step 4: Synthesis of Compound 26-7

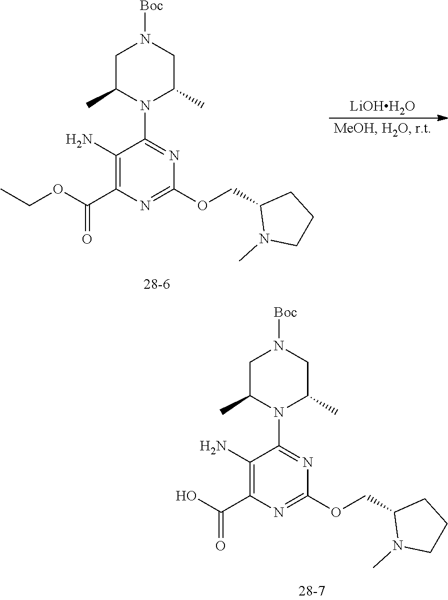 [SMILES: CCOC(=O)c1nc(OC[C@@H]2CCCN2C)nc(N2[C@@H](C)CN(C(=O)OC(C)(C)C)C[C@@H]2C)c1N ; "LiOH·H2O" ; "MeOH, H2O, r.t." ; C[C@H]1CN(C(=O)OC(C)(C)C)C[C@H](C)N1c1nc(OC[C@@H]2CCCN2C)nc(C(=O)O)c1N]

To a solution of 5-amino-6-((2S,6S)-4-(tert-butoxycarbonyl)-2,6-dimethylpiperazin-1-yl)-2-(((S)-1-methylpyrrolidin-2-yl)methoxy)pyrimidine-4-carboxylate (2.00 g, 4.06 mmol, 1.0 eq.) in MeOH (10 mL) and water (3 mL) was added LiOH·H₂O (854 mg, 20.3 mmol, 5.0 eq). The mixture was stirred at room temperature for 3 h. LCMS showed starting material was consumed and desired product formed. The mixture was acidified with aq. HCl (0.5 M) to adjust pH=6, and then concentrated to dryness to obtain 5-amino-6-((2S,6S)-4-(tert-butoxycarbonyl)-2,6-dimethylpiperazin-1-yl)-2-(((S)-1-methylpyrrolidin-2-yl)methoxy)pyrimidine-4-carboxylic acid (3.38 g, crude, 28-7), which was used directly for the next step.

LCMS (ESI, m/z): [M+1]⁺=465; RT=1.079 min.

### Step 5: Synthesis of Compound 28-9

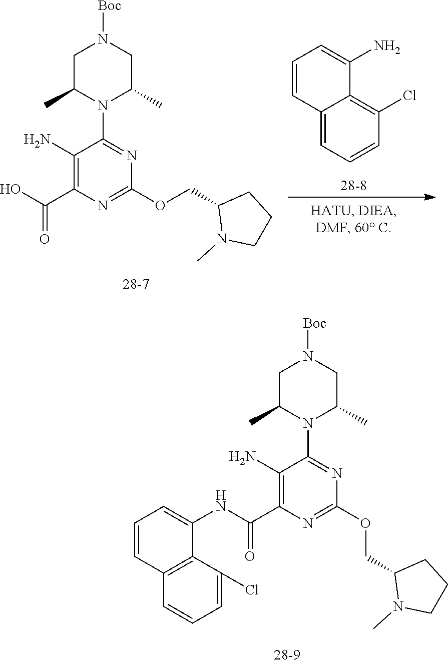 [SMILES: C[C@H]1CN(C(=O)OC(C)(C)C)C[C@H](C)N1c1nc(OC[C@@H]2CCCN2C)nc(C(=O)O)c1N ; Nc1cccc2cccc(Cl)c12 ; C[C@H]1CN(C(=O)OC(C)(C)C)C[C@H](C)N1c1nc(OC[C@@H]2CCCN2C)nc(C(=O)Nc2cccc3cccc(Cl)c23)c1N]

To a solution of 6-amino-4-chloro-1-(2,6-dimethylphenyl)pyrimidin-2(1H)-one (3.38 g, 7.3 mmol, 1.0 eq.) and 8-chloronaphthalen-1-amine (1.03 g, 5.8 mmol, 0.8 eq) in anhydrous DMF (30 mL) was added DIEA (2.82 g, 21.8 mmol, 3.0 eq.), followed by the addition of HATU (3.32 g, 8.7 mmol, 1.2 eq.). The solution was stirred at 60° C. for 1 h. LCMS showed starting material was consumed and desired product formed. The reaction mixture was cooled to room temperature, diluted with brine (120 mL) and extracted with EtOAc (80 mL×3). The combined organic fractions were washed with brine (100 mL), dried over anhydrous Na₂SO₄ and concentrated. The residue was purified by silica column chromatography eluting with DCM/MeOH (10:1, v/v) to obtain tert-butyl (3S,5S)-4-(5-amino-6-((8-chloronaphthalen-1-yl)carbamoyl)-2-(((S)-1-methylpyrrolidin-2-yl)methoxy)pyrimidin-4-yl)-3,5-dimethylpiperazine-1-carboxylate (1.30 g, 29% yield, 28-9).

LCMS (ESI, m/z): [M+1]⁺=624; RT=1.340 min.

### Step 6: Synthesis of Compound 28-11

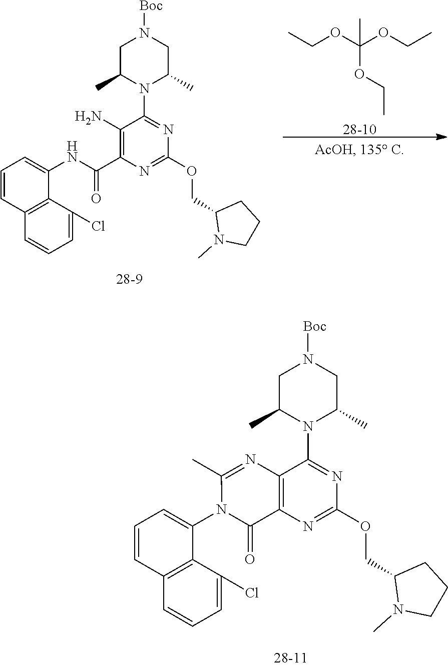

To a mixture of tert-butyl (3S,5S)-4-(5-amino-6-((8-chloronaphthalen-1-yl)carbamoyl)-2-(((S)-1-methylpyrrolidin-2-yl)methoxy)pyrimidin-4-yl)-3,5-dimethylpiperazine-1-carboxylate (1.30 g, 2.1 mmol, 1.0 eq.) and AcOH (15 mL) was added 1,1,1-triethoxyethane (5.11 g, 31.5 mmol, 15.0 eq.). The mixture was stirred at 135° C. for 3 min. LCMS howed starting material was consumed and desired product formed. The reaction mixture was cooled, quenched with aq. NaHCO₃ (120 mL) and extracted with DCM (60 mL×3). The combined organic fractions were dried over anhydrous Na₂SO₄ and concentrated. The residue was purified by silica column chromatography eluting with DCM/MeOH (10:1, v/v) to obtain tert-butyl (3S,5S)-4-(7-(8-chloronaphthalen-1-yl)-6-methyl-2-(((S)-1-methylpyrrolidin-2-yl)methoxy)-8-oxo-7,8-dihydropyrimido[5,4-d]pyrimidin-4-yl)-3,5-dimethylpiperazine-1-carboxylate (710 mg, 52% yield, 28-11).

LCMS (ESI, m/z): [M+1]⁺=648; RT=1.306 min.

### Step 7: Synthesis of Compound 28-12

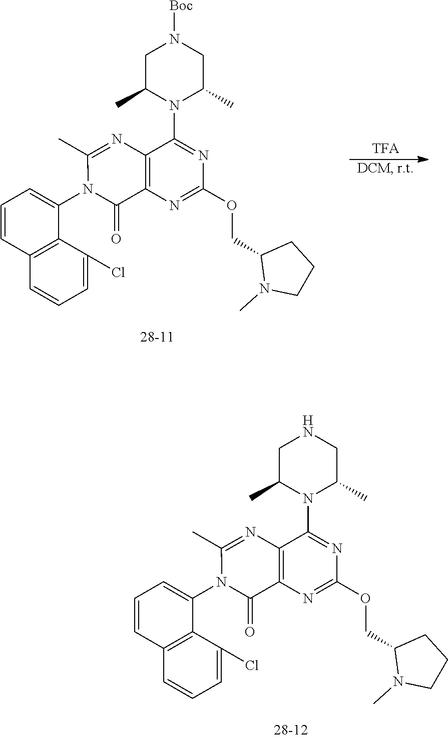 [SMILES: Cc1nc2c(N3[C@@H](C)CN(C(=O)OC(C)(C)C)C[C@@H]3C)nc(OC[C@@H]3CCCN3C)nc2c(=O)n1-c1cccc2cccc(Cl)c12 ; "DCM, r.t." ; Cc1nc2c(N3[C@@H](C)CNC[C@@H]3C)nc(OC[C@@H]3CCCN3C)nc2c(=O)n1-c1cccc2cccc(Cl)c12 ; "TFA"]

To a solution of tert-butyl 4-(1-(2,6-dimethylphenyl)-6-(2-fluorobenzamido)-2-oxo-1,2-dihydropyrimidin-4-yl)piperazine-1-carboxylate (710 mg, 1.1 mmol, 1.0 eq.) in DCM (5 mL) was added trifluoroacetic acid (2.5 mL), and the mixture was stirred at room temperature for 2 h. LCMS howed starting material was consumed and desired product formed. The reaction mixture was concentrated and the residue was treated with aq. NaHCO₃ (sat. 30 mL) to adjust pH=7-8, which was extracted with DCM (15 mL×3). The combined organic fractions were dried over anhydrous Na₂SO₄ and concentrated to obtain 3-(8-chloronaphthalen-1-yl)-8-((2S,6S)-2,6-dimethylpiperazin-1-yl)-2-methyl-6-(((S)-1-methylpyrrolidin-2-yl) methoxy)pyrimido[5,4-d]pyrimidin-4(3H)-one (550 mg, 92% yield, 28-12), which was used directly for the next step.

LCMS (ESI, m/z): [M+1]⁺=548; RT=0.697 min.

### Step 9: Synthesis of Compounds 28-a and 28-b

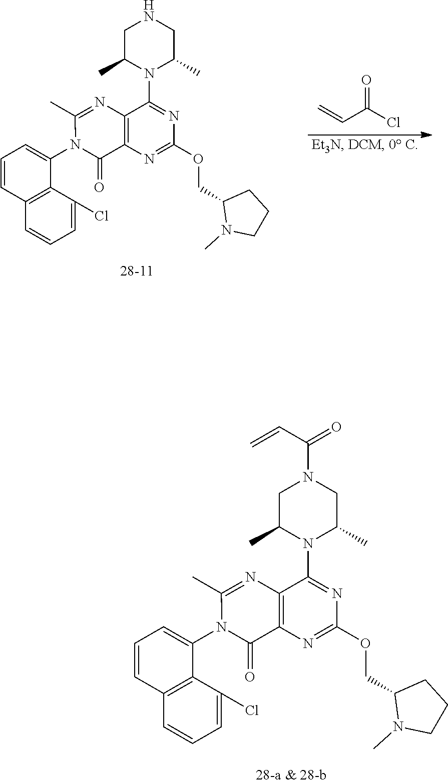

To a cooled (0° C.) solution of 3-(8-chloronaphthalen-1-yl)-8-((2S,6S)-2,6-dimethylpiperazin-1-yl)-2-methyl-6-(((S)-1-methylpyrrolidin-2-yl)methoxy)pyrimido[5,4-d]pyrimidin-4(3H)-one (550 mg, 1.0 mmol, 1.0 eq.) and Et₃N (305 mg, 3.0 mmol, 3.0 eq) in anhydrous DCM (3 mL) was added dropwise a solution of acryloyl chloride (90.5 mg, 1.0 mmol, 1.0 eq) in anhydrous DCM (0.5 mL). The mixture was stirred at 0° C. for 30 min. LCMS showed starting material was consumed and desired product formed. Water (25 mL) was added and the organic layer was separated. The aqueous layer was extracted with DCM (10 mL×3). The combined organic fractions were dried over anhydrous Na₂SO₄ and concentrated. The residue was purified by prep-TLC and then SFC to obtain 8-((2S,6S)-4-acryloyl-2,6-dimethylpiperazin-1-yl)-3-(8-chloronaphthalen-1-yl)-2-methyl-6-(((S)-1-methylpyrrolidin-2-yl)methoxy)pyrimido[5,4-d]pyrimidin-4(3H)-one (12.35 mg, 2% yield, 28-a), and 8-((2S,6S)-4-acryloyl-2,6-dimethylpiperazin-1-yl)-3-(8-chloronaphthalen-1-yl)-2-methyl-6-(((S)-1-methylpyrrolidin-2-yl)methoxy)pyrimido[5,4-d] pyrimidin-4(3H)-one (3.56 mg, 0.6% yield, 28-b).

28-a:

LCMS (ESI, m/z): [M+1]⁺=602; RT=1.787 min;

¹H NMR (400 MHz, CDCl₃) δ 8.08 (d, J=7.6 Hz, 1H), 7.93 (d, J=8.0 Hz, 1H), 7.70-7.64 (m, 1H), 7.59 (dd, J=7.4, 1.0 Hz, 1H), 7.47 (dd, J=16.2, 8.2 Hz, 2H), 6.59 (dd, J=16.8, 10.3 Hz, 1H), 6.44 (dd, J=16.8, 2.0 Hz, 1H), 5.80 (dd, J=10.3, 2.0 Hz, 1H), 5.72-5.55 (m, 1H), 4.92-4.77 (m, 2H), 4.25-4.00 (m, 3H), 3.87-3.70 (m, 4H), 3.04-2.90 (m, 4H), 2.33 (s, 1H), 2.13 (s, 3H), 1.97 (s, 3H), 1.49 (t, J=6.8 Hz, 6H).

28-b:

LCMS (ESI, m/z): [M+1]⁺=602; RT=1.793 min;

¹H NMR (400 MHz, CDCl₃) δ 8.05 (dd, J=8.3, 0.9 Hz, 1H), 7.91 (dd, J=8.3, 0.9 Hz, 1H), 7.68-7.62 (m, 1H), 7.58 (dd, J=7.5, 1.1 Hz, 1H), 7.48-7.41 (m, 2H), 6.60 (dd, J=16.8, 10.4 Hz, 1H), 6.44 (dd, J=16.8, 2.0 Hz, 1H), 5.80 (dd, J=10.3, 2.0 Hz, 1H), 5.72-5.50 (m, 1H), 4.71-4.36 (m, 2H), 4.21-4.03 (m, 2H), 3.84-3.70 (m, 2H), 3.48-2.97 (m, 2H), 2.88-2.26 (m, 5H), 2.17 (s, 1H), 2.11 (s, 3H), 1.89 (s, 3H), 1.48 (dd, J=6.5, 4.4 Hz, 6H).

### Example 29

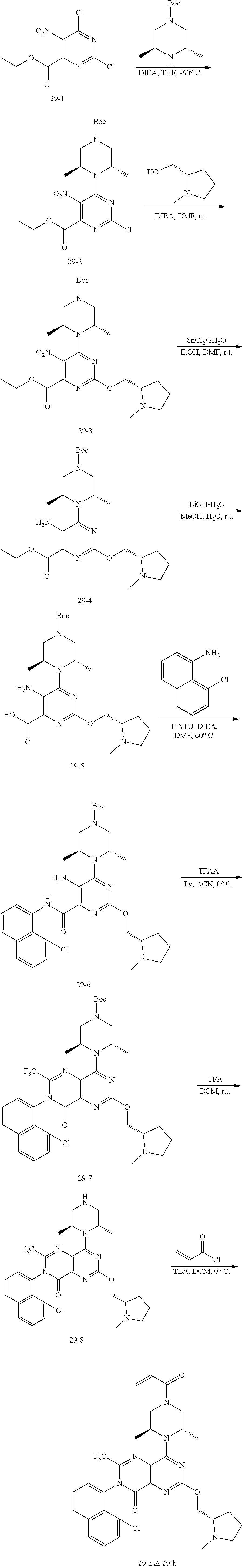

### Step 1: Synthesis of Compound 29-2

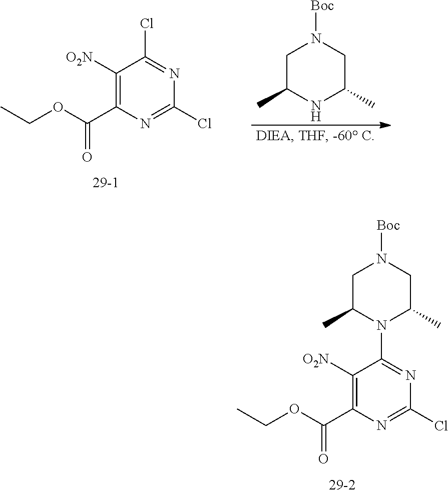

To a cooled (−60° C.) solution of ethyl 2,6-dichloro-5-nitropyrimidine-4-carboxylate (6.21 g, 23.4 mmol, 1.0 eq.) in anhydrous THF (40 mL) was added dropwise a solution of tert-butyl (3S,5S)-3,5-dimethylpiperazine-1-carboxylate (5.00 g, 23.4 mmol, 1.0 eq.) and DIEA (4.52 g, 35.0 mmol, 1.5 eq.) in anhydrous THF (30 mL). The mixture was stirred at −60° C. for 1 h. TLC showed the reaction was completed. The mixture was concentrated in vacuo and the residue was purified by silica column chromatography eluting with Pet.ether/EtOAc (2:1, v/v) to obtain ethyl 6-((2S,6S)-4-(tert-butoxycarbonyl)-2,6-dimethylpiperazin-1-yl)-2-chloro-5-nitropyrimidine-4-carboxylate (8.80 g, 85% yield, 29-2).

### Step 2: Synthesis of Compound 29-3

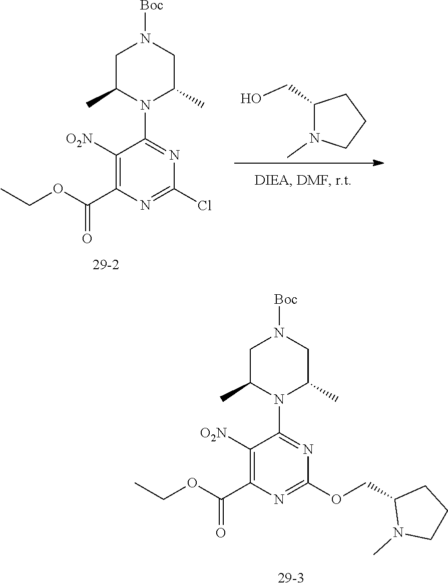

To a solution of ethyl 6-((2S,6S)-4-(tert-butoxycarbonyl)-2,6-dimethylpiperazin-1-yl)-2-chloro-5-nitropyrimidine-4-carboxylate (8.80 g, 19.7 mmol, 1.0 eq.) and DIEA (5.13 g, 39.7 mmol, 2.0 eq.) in anhydrous DMF (30 mL) was added (S)-(1-methylpyrrolidin-2-yl)methanol (3.43 g, 29.8 mmol, 1.5 eq). The mixture was stirred at room temperature for 3 h. LCMS showed starting material was consumed and desired product formed. The solution was diluted with brine (120 mL) and extracted with EtOAc (80 mL×2). The combined organic layers were dried over anhydrous Na₂SO₄ and concentrated. The residue was purified by silica column chromatography eluting with DCM/MeOH (15:1, v/v) to obtain ethyl 6-((2S,6S)-4-(tert-butoxycarbonyl)-2,6-dimethylpiperazin-1-yl)-2-(((S)-1-methylpyrrolidin-2-yl)methoxy)-5-nitropyrimidine-4-carboxylate (10.00 g, 96% yield, 29-3).

LCMS (ESI, m/z): [M+1]⁺=523; RT=1.125 min.

### Step 3: Synthesis of Compound 29-4

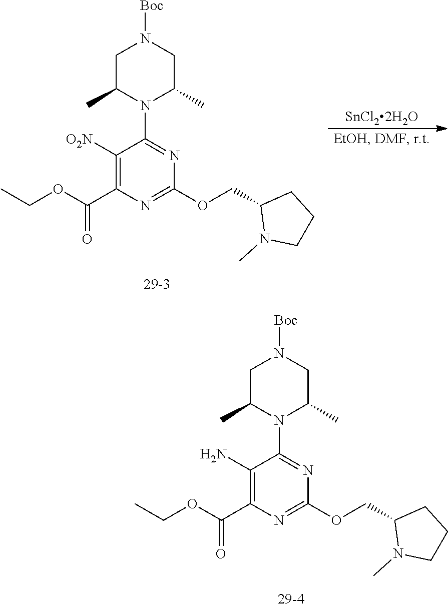 [SMILES: CCOC(=O)c1nc(OC[C@@H]2CCCN2C)nc(N2[C@@H](C)CN(C(=O)OC(C)(C)C)C[C@@H]2C)c1[N+](=O)[O-] ; "EtOH, DMF, r.t." ; "SnCl2·2H2O" ; CCOC(=O)c1nc(OC[C@@H]2CCCN2C)nc(N2[C@@H](C)CN(C(=O)OC(C)(C)C)C[C@@H]2C)c1N]

To a solution of ethyl 6-((2S,6S)-4-(tert-butoxycarbonyl)-2,6-dimethylpiperazin-1-yl)-2-(((S)-1-methylpyrrolidin-2-yl)methoxy)-5-nitropyrimidine-4-carboxylate (10.00 g, 19.2 mmol, 1.0 eq.) in anhydrous EtOH (50 mL)/DMF (50 mL) was added SnCl₂·2H₂O (21.66 g, 96.0 mmol, 5.0 eq.). The solution was stirred at room temperature for 3 hours. LCMS showed starting material was consumed and desired product formed. The reaction mixture was concentrated to remove EtOH and then diluted with EtOAc (150 mL), followed by the addition of aq. NaHCO₃ (sat. 200 mL). The resulting mixture was filtered through celite. The organic layer of the filtrate was separated and the aqueous layer was extracted with EtOAc (120 mL×2). The combined organic fractions were washed with brine (200 mL), dried over anhydrous Na₂SO₄ and concentrated. The residue was purified by silica column chromatography eluting with DCM/MeOH (10:1, v/v) to obtain ethyl 5-amino-6-((2S,6S)-4-(tert-butoxycarbonyl)-2,6-dimethylpiperazin-1-yl)-2-(((S)-1-methylpyrrolidin-2-yl)methoxy)pyrimidine-4-carboxylate (2.00 g, 21% yield, 29-4).

LCMS (ESI, m/z): [M+1]⁺=451; RT=0.928 min.

### Step 4: Synthesis of Compound 29-5

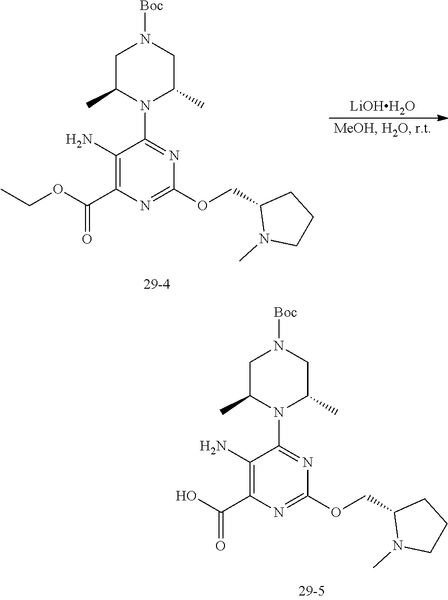 [SMILES: CCOC(=O)c1nc(OC[C@@H]2CCCN2C)nc(N2[C@@H](C)CN(C(=O)OC(C)(C)C)C[C@@H]2C)c1N ; "LiOH·H2O" ; "MeOH, H2O, r.t." ; C[C@H]1CN(C(=O)OC(C)(C)C)C[C@H](C)N1c1nc(OC[C@@H]2CCCN2C)nc(C(=O)O)c1N]

To a solution of ethyl 5-amino-6-((2S,6S)-4-(tert-butoxycarbonyl)-2,6-dimethylpiperazin-1-yl)-2-(((S)-1-methylpyrrolidin-2-yl) methoxy) pyrimidine-4-carboxylate (630 mg, 1.28 mmol, 1.0 eq.) in MeOH (6.0 mL) and H₂O (1 mL) was added LiOH·H₂O (269 mg, 6.40 mmol, 5.0 eq.). The mixture was stirred at room temperature for 1 h. LCMS showed starting material was consumed and desired product formed. The reaction mixture was acidified with aq. HCl (1.0 M) to pH=6, and then concentrated to dryness to obtain 5-amino-6-((2S,6S)-4-(tert-butoxycarbonyl)-2,6-dimethylpiperazin-1-yl)-2-(((S)-1-methylpyrrolidin-2-yl)methoxy) pyrimidine-4-carboxylic acid (720 mg, crude, 29-5), which was used directly for the next step.

LCMS (ESI, m/z): [M+1]⁺=465; RT=1.096 min.

### Step 5: Synthesis of Compound 29-6

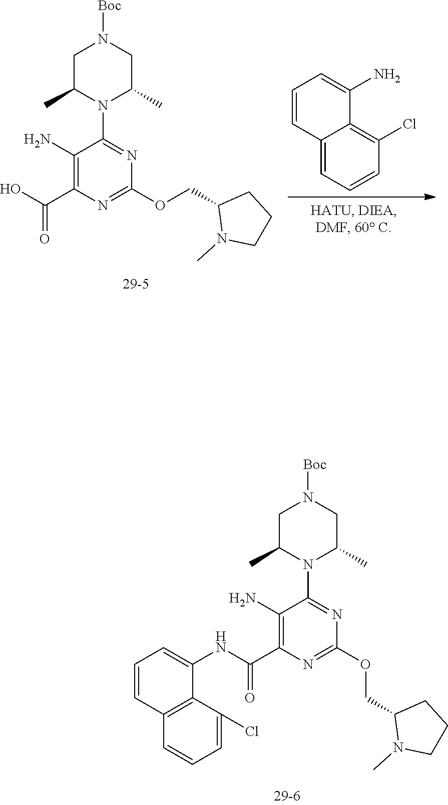 [SMILES: C[C@H]1CN(C(=O)OC(C)(C)C)C[C@H](C)N1c1nc(OC[C@@H]2CCCN2C)nc(C(=O)O)c1N ; Nc1cccc2cccc(Cl)c12 ; C[C@H]1CN(C(=O)OC(C)(C)C)C[C@H](C)N1c1nc(OC[C@@H]2CCCN2C)nc(C(=O)Nc2cccc3cccc(Cl)c23)c1N]

To a mixture of 5-amino-6-((2S,6S)-4-(tert-butoxycarbonyl)-2,6-dimethylpiperazin-1-yl)-2-(((S)-1-methylpyrrolidin-2-yl) methoxy) pyrimidine-4-carboxylic acid (720 mg, 1.55 mmol, 1.0 eq.) and 8-chloronaphthalen-1-amine (192 mg, 1.09 mmol, 0.7 eq.) in anhydrous DMF (10 mL), was added DIEA (600 mg, 4.65 mmol, 3.0 eq.), followed by the addition of HATU (649 mg, 1.71 mmol, 1.1 eq.). The mixture was stirred at 60° C. under Ar for 1 h. LCMS showed starting material was consumed and desired product formed. The reaction mixture was cooled to room temperature, diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with DCM/MeOH (10:1, v/v) to obtain tert-butyl (3S,5S)-4-(5-amino-6-((8-chloronaphthalen-1-yl) carbamoyl)-2-(((S)-1-methylpyrrolidin-2-yl) methoxy) pyrimidin-4-yl)-3,5-dimethylpiperazine-1-carboxylate (300 mg, 44% yield, 29-6).

LCMS (ESI, m/z): [M+1]⁺=624; RT=1.233 min.

### Step 6: Synthesis of Compound 29-7

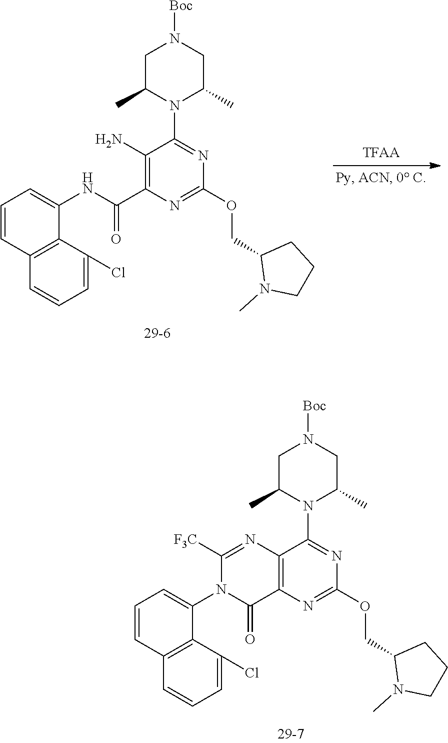 [SMILES: C[C@H]1CN(C(=O)OC(C)(C)C)C[C@H](C)N1c1nc(OC[C@@H]2CCCN2C)nc(C(=O)Nc2cccc3cccc(Cl)c23)c1N ; "TFAA" ; C[C@H]1CN(C(=O)OC(C)(C)C)C[C@H](C)N1c1nc(OC[C@@H]2CCCN2C)nc2c(=O)n(-c3cccc4cccc(Cl)c34)c(C(F)(F)F)nc12]

To a solution of tert-butyl (3S,5S)-4-(5-amino-6-((8-chloronaphthalen-1-yl) carbamoyl)-2-(((S)-1-methylpyrrolidin-2-yl) methoxy) pyrimidin-4-yl)-3,5-dimethylpiperazine-1-carboxylate (200 mg, 0.32 mmol, 1.0 eq.) in anhydrous ACN (10.0 mL) was added pyridine (254 mg, 3.20 mmol, 10.0 eq.), followed by the addition of TFAA (202 mg, 1.92 mmol, 6.0 eq.). The mixture was stirred at 0° C. for 30 min. LCMS showed starting material was consumed and desired product formed. The reaction mixture was quenched with aq. NH₄Cl (sat. 25 mL) and extracted with EtOAc (15 mL×3). The combined organic fractions were washed with brine (20 mL), dried over anhydrous Na₂SO₄ and concentrated. The residue was purified by prep-TLC eluting with DCM/MeOH (10:1, v/v) to obtain tert-butyl (3S,5S)-4-(7-(8-chloronaphthalen-1-yl)-2-(((S)-1-methylpyrrolidin-2-yl) methoxy)-8-oxo-6-(trifluoromethyl)-7,8-dihydropyrimido[5,4-d] pyrimidin-4-yl)-3,5-dimethylpiperazine-1-carboxylate (110 mg, 49% yield, 29-7).

LCMS (ESI, m/z): [M+1]⁺=702; RT=1.189 min.

### Step 7: Synthesis of Compound 29-8

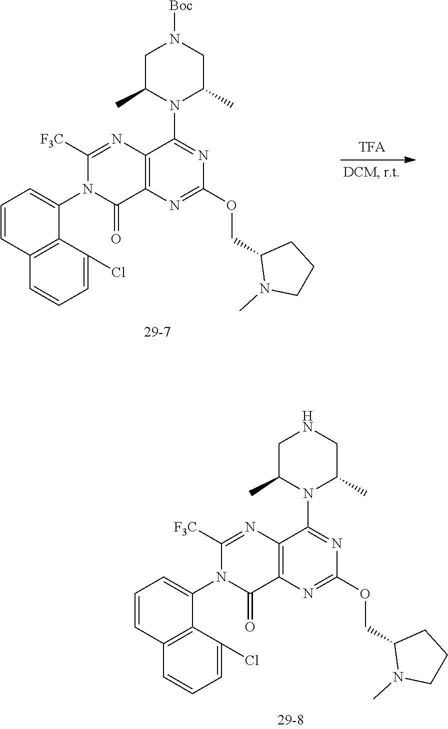 [SMILES: C[C@H]1CN(C(=O)OC(C)(C)C)C[C@H](C)N1c1nc(OC[C@@H]2CCCN2C)nc2c(=O)n(-c3cccc4cccc(Cl)c34)c(C(F)(F)F)nc12 ; C[C@H]1CNC[C@H](C)N1c1nc(OC[C@@H]2CCCN2C)nc2c(=O)n(-c3cccc4cccc(Cl)c34)c(C(F)(F)F)nc12]

To a solution of tert-butyl (3S,5S)-4-(7-(8-chloronaphthalen-1-yl)-2-(((S)-1-methylpyrrolidin-2-yl) methoxy)-8-oxo-6-(trifluoromethyl)-7,8-dihydropyrimido[5,4-d] pyrimidin-4-yl)-3,5-dimethylpiperazine-1-carboxylate (100 mg, 0.14 mmol) in DCM (5.0 mL) was added TFA (2.0 mL), and the mixture was stirred at room temperature for 1 h. LCMS showed starting material was consumed and desired product formed. The resulting mixture was concentrated to obtain 3-(8-chloronaphthalen-1-yl)-8-((2S,6S)-2,6-dimethylpiperazin-1-yl)-6-(((S)-1-methylpyrrolidin-2-yl) methoxy)-2-(trifluoromethyl) pyrimido [5,4-d] pyrimidin-4(3H)-one (TFA salt, 100 mg, crude, 29-8), which was used directly for the next step.

LCMS (ESI, m/z): [M+1]⁺=602.3; RT=0.955 min.

### Step 8: Synthesis of Compounds 29a and 29b

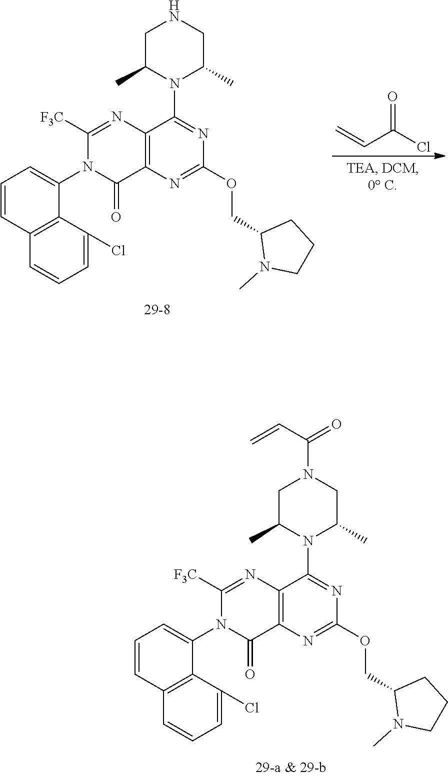

To a cooled (0° C.) solution of 3-(8-chloronaphthalen-1-yl)-8-((2S,6S)-2,6-dimethylpiperazin-1-yl)-6-(((S)-1-methylpyrrolidin-2-yl) methoxy)-2-(trifluoromethyl) pyrimido [5,4-d] pyrimidin-4(3H)-one (100 mg, 0.14 mmol, 1.0 eq.) and Et₃N (71 mg, 0.70 mmol, 5.0 eq.) in DCM (3 mL) was added dropwise a solution of acryloyl chloride (19 mg, 0.21 mmol, 1.5 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((2S,6S)-4-acryloyl-2,6-dimethylpiperazin-1-yl)-3-(8-chloronaphthalen-1-yl)-6-(((S)-1-methylpyrrolidin-2-yl) methoxy)-2-(trifluoro methyl)pyrimido[5,4-d]pyrimidin-4(3H)-one (4.5 mg, 9% yield, 29-a), and 8-((2S,6S)-4-acryloyl-2,6-dimethylpiperazin-1-yl)-3-(8-chloronaphthalen-1-yl)-6-(((S)-1-methylpyrrolidin-2-yl) methoxy)-2-(trifluoro methyl)pyrimido[5,4-d] pyrimidin-4(3H)-one (5.6 mg, 11% yield, 29-b).

29-a:

LCMS (ESI, m/z): [M+1]⁺=656; RT=2.107 min;

¹H NMR (400 MHz, CDCl₃) δ 8.08 (d, J=8.0 Hz, 1H), 7.91 (d, J=8.1 Hz, 1H), 7.66-7.60 (m, 1H), 7.56 (d, J=7.4 Hz, 1H), 7.48-7.40 (m, 2H), 6.64-6.54 (m, 1H), 6.50-6.41 (m, 1H), 5.84-5.77 (m, 1H), 5.46-4.57 (m, 3H), 4.23-4.05 (m, 2H), 3.87-3.71 (m, 2H), 2.85 (s, 3H), 2.34-1.92 (m, 5H), 1.49 (d, J=6.5 Hz, 6H), 1.28-1.21 (m, 3H);

¹⁹F NMR (400 MHz, CDCl₃) δ −64.7.

29-b:

LCMS (ESI, m/z): [M+1]⁺=656; RT=2.100 min;

¹H NMR (400 MHz, CDCl₃) δ 8.08 (d, J=7.9 Hz, 1H), 7.90 (d, J=7.9 Hz, 1H), 7.63 (t, J=7.8 Hz, 1H), 7.58 (d, J=6.8 Hz, 1H), 7.49-7.42 (m, 2H), 6.64-6.55 (m, 1H), 6.51-6.41 (m, 1H), 5.85-5.78 (m, 1H), 5.49-4.45 (m, 3H), 4.24-4.05 (m, 2H), 3.86-3.70 (m, 2H), 2.69 (s, 3H), 2.20-1.85 (m, 5H), 1.55-1.49 (m, 6H), 1.29-1.22 (m, 3H);

¹⁹F NMR (400 MHz, CDCl₃) δ −64.7.

### Example 30

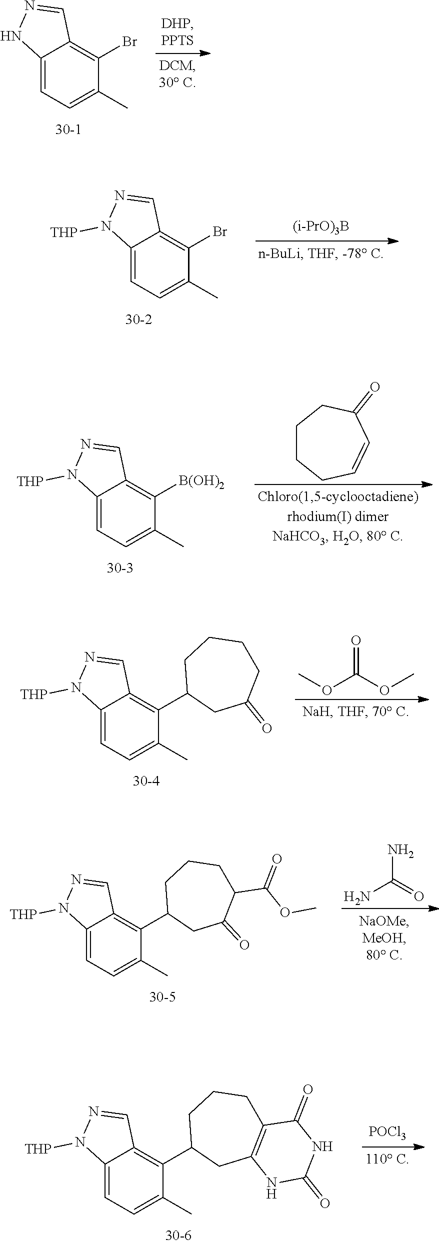

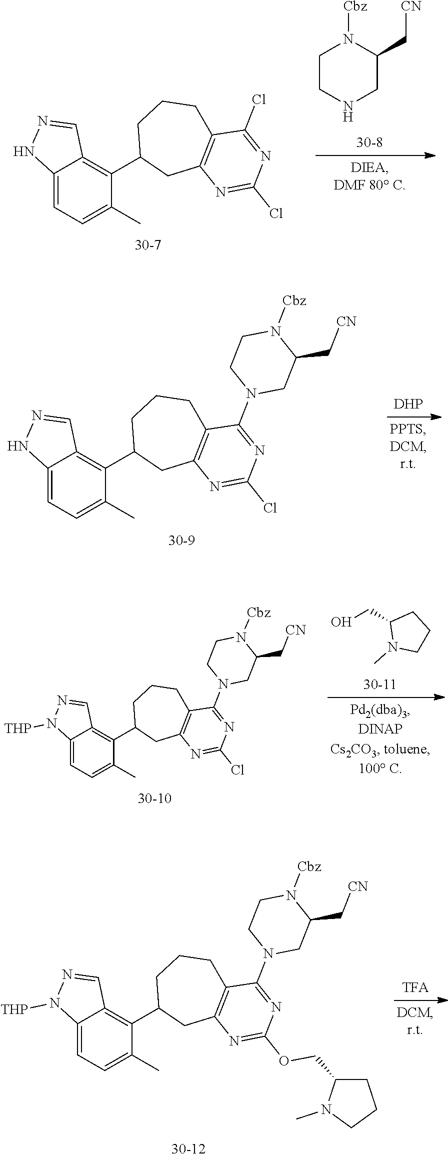

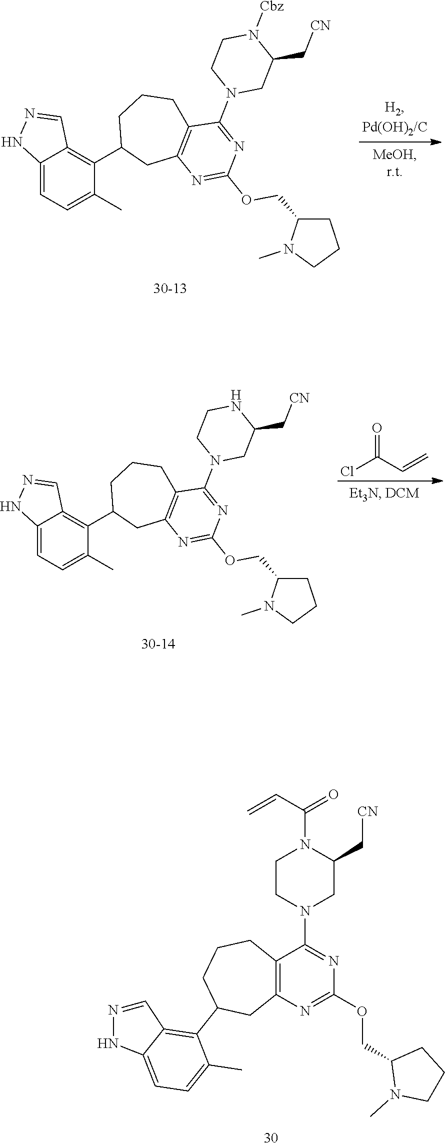

### Step 1: Synthesis of Compound 30-2

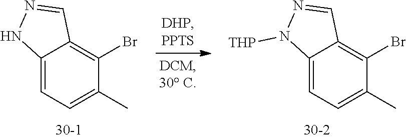

To a solution of 4-bromo-5-methyl-1H-indazole (14.0 g, 66.67 mmol, 1.0 eq.) in anhydrous DCM (30 mL) was added PPTS (1.68 g, 6.68 mmol 0.1 eq.) at room temperature (r.t.). Then DHP (16.83 g, 200.02 mmol, 3 eq.) was added in one portion. The reaction mixture was stirred at 30° C. overnight. LCMS analysis showed starting material was consumed and desired product formed. The reaction was quenched with H₂O (50 mL) and the layers were separated. The aqueous layer was extracted with DCM (30 mL×3). The combined organics were washed with brine (30 mL), dried over anhydrous Na₂SO₄ and concentrated. The residue was purified by silica column chromatography eluting with EtOAc/Pet.ether (15%, v/v) to obtain 4-bromo-5-methyl-1-(tetrahydro-2H-pyran-2-yl)-1H-indazole (10.8 g, 55% yield, 30-2).

LCMS (ESI, m/z). [M+1]⁺=295; RT=2.158 min.

### Step 2: Synthesis of Compound 30-3

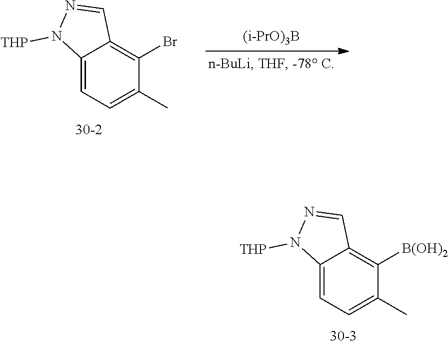

To a cooled (−78° C.) solution of 4-bromo-5-methyl-1-(tetrahydro-2H-pyran-2-yl)-1H-indazole (5.0 g, 17.00 mmol, 1.0 eq.) in anhydrous THF (30 mL) was added (i-PrO)₃B (6.4 g, 34.00 mmol, 2.0 eq.). Then n-BuLi (2.5 mol/L in THF, 13.0 mL, 31.46 mmol, 1.85 eq.) was added dropwise to the above solution over a period of 30 min, maintaining the reaction temperature between −70° C. and −65° C. After addition, the reaction was stirred at −78° C. for 3 h. LCMS analysis showed starting material was consumed and desired product formed. The reaction mixture was quenched with a solution of saturated aq.NH₄Cl (sat.20 mL) and diluted with MTBE (30 mL). The layers were separated and the aqueous layer was extracted with MTBE (30 mL×3). The combined organics were washed with brine (50 mL), dried over anhydrous Na₂SO₄ and concentrated. The residue was dissolved in MTBE (10 mL). Pet.ether was added dropwise to the solution at 0° C. White solid precipitated during the Pet.ether addition. The resultant suspension was filtered and the filter cake was washed with Pet.ether (30 mL). The filter cake was dried under vacuum to obtain (5-methyl-1-(tetrahydro-2H-pyran-2-yl)-1H-indazol-4-yl)boronic acid (4.2 g, 95% yield, 30-3), which was used directly for the next step.

LCMS (ESI, m/z): [M+1]⁺=261; RT=1.242 min.

### Step 3: Synthesis of Compound 30-4

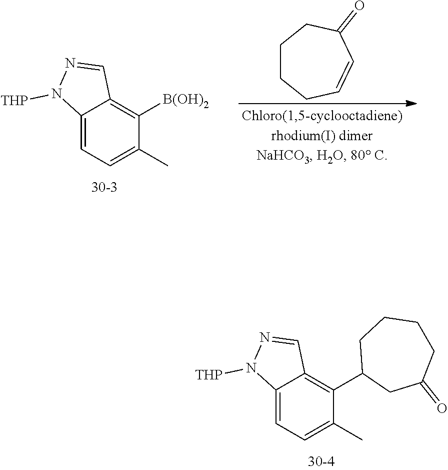

To a mixture of (5-methyl-1-(tetrahydro-2H-pyran-2-yl)-1H-indazol-4-yl)boronic acid (3.0 g, 11.54 mmol, 1.0 eq.) and cyclohept-2-en-1-one (3.8 g, 34.62 mmol, 3.0 eq.) in H₂O (20 mL) were added NaHCO₃ (1.94 g, 23.08 mmol, 2.0 eq.) and chloro(1,5-cyclooctadiene)rhodium(I) dimer (0.28 g, 0.58 mmol, 0.05 eq.). The mixture was stirred at 80° C. under Ar overnight. LCMS analysis showed the starting material was consumed and desired product formed. The reaction mixture was diluted with EtOAc (30 mL) and the layers were separated. The aqueous layer was extracted with EtOAc (30 mL×3). The combined organics were washed with brine (30 mL), dried over anhydrous Na₂SO₄ and concentrated. The residue was purified by silica column chromatography eluting with EtOAc/Pet.ether (20%, v/v) to obtain 3-(5-methyl-1-(tetrahydro-2H-pyran-2-yl)-1H-indazol-4-yl)cycloheptan-1-one (1.3 g, 35% yield, 30-4).

LCMS (ESI, m/z): [M+1]⁺=327; RT=1.662 min.

### Step 4: Synthesis of Compound 30-5

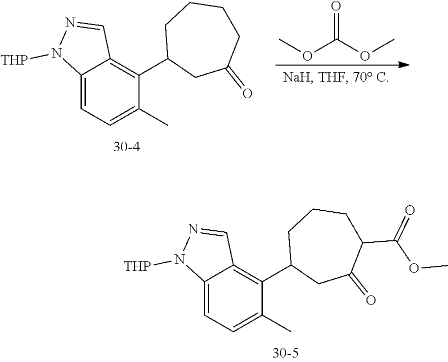

To a solution of 3-(5-methyl-1-(tetrahydro-2H-pyran-2-yl)-1H-indazol-4-yl)cycloheptan-1-one (763 mg, 2.34 mmol, 1.0 eq.) and dimethyl carbonate (4.0 mL, 46.81 mmol, 20.0 eq.) in THF (5.0 mL) was added NaH (60% dispersion in mineral oil, 140 mg, 5.85 mmol, 2.5 eq.), and the mixture was stirred at 70° C. for 2 h. LCMS analysis showed starting material was consumed and desired product formed. The reaction mixture was quenched with H₂O (10.0 mL) and extracted with EtOAc (20 mL×3). The combined organics were washed with brine (20 mL), dried over anhydrous Na₂SO₄ and concentrated. The residue was purified by silica column chromatography eluting with EtOAc/Pet.ether (20%, v/v) to obtain methyl 4-(5-methyl-1-(tetrahydro-2H-pyran-2-yl)-1H-indazol-4-yl)-2-oxocycloheptane-1-carboxylate (684 mg, 76%, 30-5).

LCMS (ESI, m/z): [M+1]⁺=385; RT=1.918 min & 2.315 min

### Step 5: Synthesis of Compound 30-6

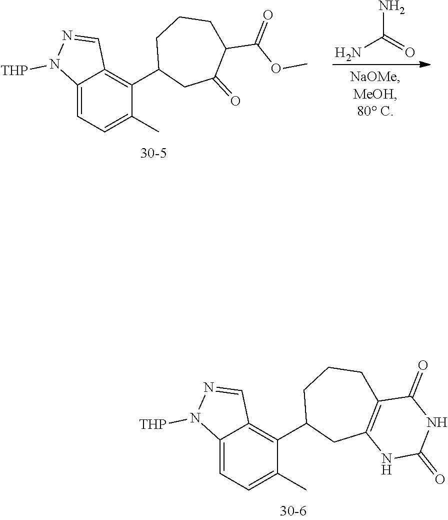

To a solution of methyl 4-(5-methyl-1-(tetrahydro-2H-pyran-2-yl)-1H-indazol-4-yl)-2-oxocycloheptane-1-carboxylate (1.74 g, 4.52 mmol, 1.0 eq.) and urea (1.09 g, 18.1 mmol, 4.0 eq.) in anhydrous MeOH (20 mL) was added NaOMe (1.0 M in MeOH, 13.6 mL, 13.6 mmol, 3.0 eq.). The reaction mixture was stirred at 80° C. under Ar overnight. LCMS analysis showed starting material was consumed and desired product formed. The reaction mixture was cooled to r.t. and concentrated to dryness. The residue was purified by silica column chromatography eluting with MeOH/DCM (10%, v/v) to obtain 8-(5-methyl-1-(tetrahydro-2H-pyran-2-yl)-1H-indazol-4-yl)-1,5,6,7,8,9-hexahydro-2H-cyclohepta[d]pyrimidine-2,4(3H)-dione (732 mg, 41%, 30-6).

LCMS (ESI, m/z): [M+1]⁺=789; RT=1.507 min.

### Step 6: Synthesis of Compound 30-7

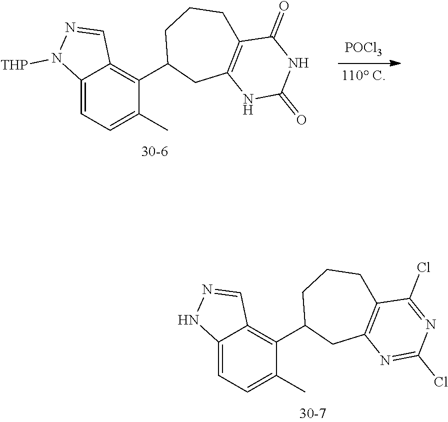

A mixture of 8-(5-methyl-1-(tetrahydro-2H-pyran-2-yl)-1H-indazol-4-yl)-1,5,6,7,8,9-hexahydro-2H-cyclohepta[d]pyrimidine-2,4(3H)-dione (732 mg, 1.86 mmol) and POCl₃ (15 mL) was stirred at 110° C. for 1 h. LCMS analysis showed starting material was consumed and desired product formed. The reaction mixture was concentrated to dryness. The residue was dissolved in DCM (50 mL) and basified with DIEA to pH=8-9. The organic layer was washed with H₂O (15 mL×2), dried over anhydrous Na₂SO₄ and concentrated to obtain 2,4-dichloro-8-(5-methyl-1H-indazol-4-yl)-6,7,8,9-tetrahydro-5H-cyclohepta[d]pyrimidine (1.40 g, crude, 30-7), which was used directly for the next step.

LCMS (ESI, m/z): [M+1]⁺=347; RT=1.972 min.

### Step 7: Synthesis of Compound 30-9

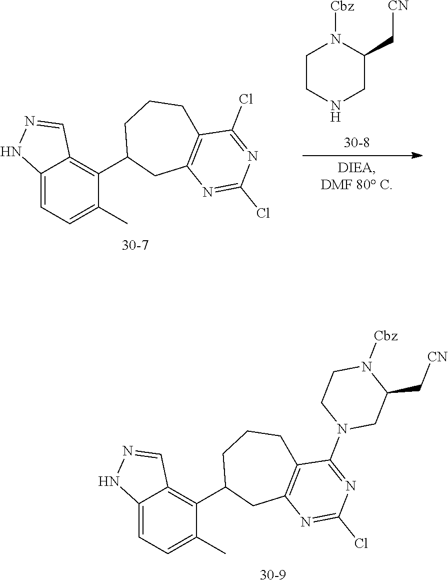 [SMILES: Cc1ccc2[nH]ncc2c1C1CCCc2c(Cl)nc(Cl)nc2C1 ; N#CC[C@H]1CNCCN1C(=O)OCc1ccccc1 ; Cc1ccc2[nH]ncc2c1C1CCCc2c(nc(Cl)nc2N2CCN(C(=O)OCc3ccccc3)[C@@H](CC#N)C2)C1]

To a solution of 2,4-dichloro-8-(5-methyl-1H-indazol-4-yl)-6,7,8,9-tetrahydro-5H-cyclohepta[d]pyrimidine (1.40 g, 4.03 mmol, 1.0 eq.) and benzyl (S)-2-(cyanomethyl)piperazine-1-carboxylate (1.57 g, 6.05 mmol, 1.5 eq.) in anhydrous DMF (14 mL) was added DIEA (3.4 mL, 20.57 mmol, 5.1 eq.). The mixture was stirred at 80° C. for 2 h. LCMS analysis showed starting material was consumed and desired product formed. The reaction mixture was concentrated to dryness. The residue was diluted with water (50 mL) and extracted with DCM (25 mL×3). The combined organics were washed with brine (30 mL), dried over anhydrous Na₂SO₄ and concentrated. The residue was purified by silica column chromatography eluting with EtOAc/Pet.ether (30% to 70%, v/v) to obtain benzyl (2S)-4-(2-chloro-8-(5-methyl-1H-indazol-4-yl)-6,7,8,9-tetrahydro-5H-cyclohepta[d]pyrimidin-4-yl)-2-(cyanomethyl)piperazine-1-carboxylate (235 mg, 10%, 30-9).

LCMS (ESI, m/z): [M+1]⁺=570; RT=1.957 min & 2.185 min.

### Step 8: Synthesis of Compound 30-10

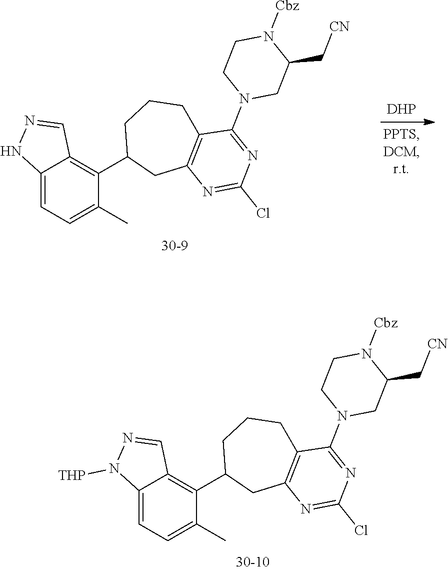

To a solution of (2S)-4-(2-chloro-8-(5-methyl-1H-indazol-4-yl)-6,7,8,9-tetrahydro-5H-cyclohepta[d]pyrimidin-4-yl)-2-(cyanomethyl)piperazine-1-carboxylate (235 mg, 0.412 mmol, 1.0 eq.) in anhydrous DCM (5 mL) was added PPTS (16 mg, 0.064 mmol, 0.15 eq.), followed by the addition of DHP (139 mg, 1.65 mmol, 4.0 eq.) in one portion. The mixture was stirred at r.t. for 20 h. LCMS analysis showed most starting material was consumed and desired product formed. The reaction mixture was concentrated and the residue was purified by prep-TLC eluting with EtOAc/Pet.ether (2:1, v/v) to obtain (2S)-4-(2-chloro-8-(5-methyl-1-(tetrahydro-2H-pyran-2-yl)-1H-indazol-4-yl)-6,7,8,9-tetrahydro-5H-cyclohepta[d]pyrimidin-4-yl)-2-(cyanomethyl)piperazine-1-carboxylate (236 mg, 87% yield, 30-10).

LCMS (ESI, m/z): [M+1]⁺=654; RT=1.940 min & 2.107 min.

### Step 9: Synthesis of Compound 30-12

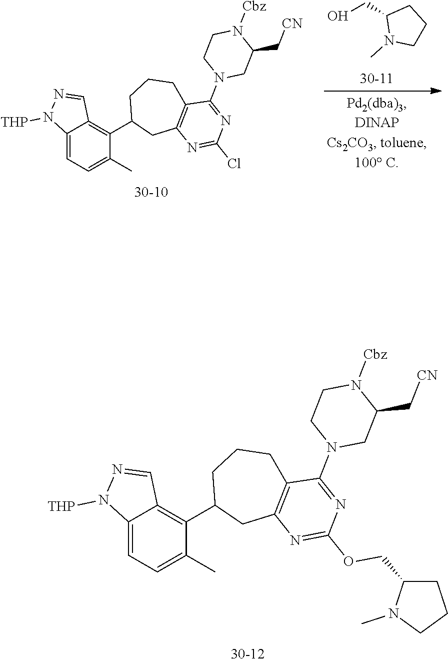

To a mixture of (2S)-4-(2-chloro-8-(5-methyl-1-(tetrahydro-2H-pyran-2-yl)-1H-indazol-4-yl)-6,7,8,9-tetrahydro-5H-cyclohepta[d]pyrimidin-4-yl)-2-(cyanomethyl)piperazine-1-carboxylate (236 mg, 0.361 mmol, 1.0 eq.), (S)-(1-methylpyrrolidin-2-yl)methanol (125 mg, 1.08 mmol, 3.0 eq.) and Cs₂CO₃ (353 mg, 1.08 mmol, 3.0 eq.) in toluene (10 mL) were added Pd₂(dba)₃ (33 mg, 0.0361 mmol, 0.1 eq) and BINAP(22 mg, 0.0361 mmol, 0.1 eq.). The mixture was stirred at 100° C. under Ar for 10 h. LCMS analysis showed most starting material was consumed and desired product formed. The reaction mixture was cooled to r.t. and filtered through celite. The filtrate was concentrated and the residue was purified by prep-TLC eluting with MeOH/DCM (1:10, v/v) to obtain benzyl (2S)-2-(cyanomethyl)-4-(8-(5-methyl-1-(tetrahydro-2H-pyran-2-yl)-1H-indazol-4-yl)-2-(((S)-1-methylpyrrolidin-2-yl)methoxy)-6,7,8,9-tetrahydro-5H-cyclohepta[d]pyrimidin-4-yl)piperazine-1-carboxylate (76 mg, 29% yield, 30-12).

LCMS (ESI, m/z): [M+1]⁺=733; RT=1.162 min.

### Step 10: Synthesis of Compound 30-13

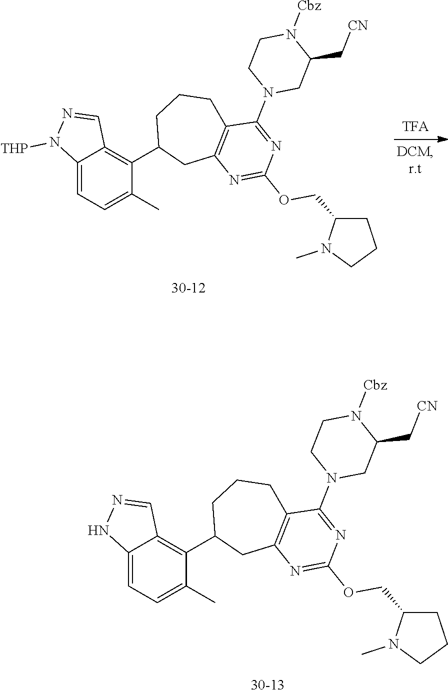

To a solution of (2S)-2-(cyanomethyl)-4-(8-(5-methyl-1-(tetrahydro-2H-pyran-2-yl)-1H-indazol-4-yl)-2-(((S)-1-methylpyrrolidin-2-yl)methoxy)-6,7,8,9-tetrahydro-5H-cyclohepta[d]pyrimidin-4-yl)piperazine-1-carboxylate (76 mg, 0.104 mmol) in anhydrous DCM (3.0 mL) was added TFA (1.0 mL), and the mixture was stirred at r.t. for 2 h. LCMS analysis showed starting material was consumed and desired product formed. The reaction mixture was basified with aq. NaHCO₃ (sat. 15 mL) to pH=7-8. The organic layer was separated and the aqueous layer was extracted with DCM (8 mL×2). The combined organics were washed with brine (10 mL), dried over anhydrous Na₂SO₄ and concentrated. The residue was purified by prep-TLC eluting with MeOH/DCM (1:10, v/v) to obtain benzyl (2S)-2-(cyanomethyl)-4-(8-(5-methyl-1H-indazol-4-yl)-2-(((S)-1-methyl pyrrolidin-2-yl)methoxy)-6,7,8,9-tetrahydro-5H-cyclohepta[d]pyrimidin-4-yl)piperazine-1-carboxylate (51 mg, 75% yield, 30-13).

LCMS (ESI, m/z): [M+1]⁺=649; RT=1.153 min.

### Step 11: Synthesis of Compound 30-14

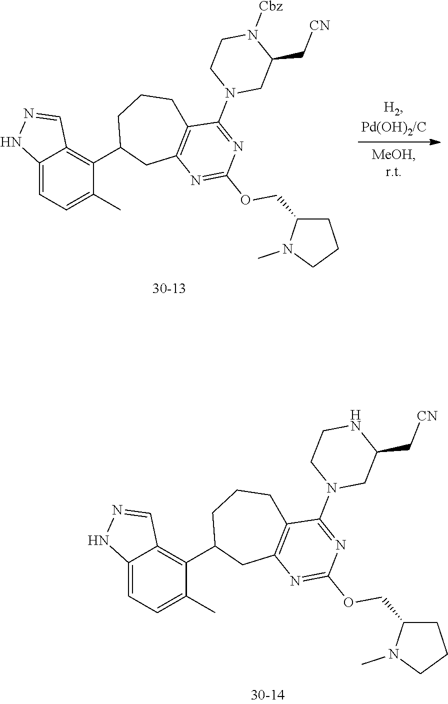 [SMILES: Cc1ccc2[nH]ncc2c1C1CCCc2c(nc(OC[C@@H]3CCCN3C)nc2N2CCN(C(=O)OCc3ccccc3)[C@@H](CC#N)C2)C1 ; Cc1ccc2[nH]ncc2c1C1CCCc2c(nc(OC[C@@H]3CCCN3C)nc2N2CCN[C@@H](CC#N)C2)C1]

To a solution of (2S)-2-(cyanomethyl)-4-(8-(5-methyl-1H-indazol-4-yl)-2-(((S)-1-methyl pyrrolidin-2-yl)methoxy)-6,7,8,9-tetrahydro-5H-cyclohepta[d]pyrimidin-4-yl)piperazine-1-carboxylate (50 mg, 0.077 mmol, 1.0 eq.) in MeOH (5.0 mL) was added Pd(OH)₂/C (10%, w/w), and the mixture was stirred at r.t. under H₂ (balloon) for 1.5 h. LCMS analysis showed starting material was consumed and desired product formed. The reaction mixture was filtered through celite. The filtrate was concentrated to dryness to obtain 2-((2S)-4-(8-(5-methyl-1H-indazol-4-yl)-2-(((S)-1-methylpyrrolidin-2-yl)methoxy)-6,7,8,9-tetrahydro-5H-cyclohepta[d]pyrimidin-4-yl)piperazin-2-yl)acetonitrile (36 mg, 91% yield, 30-14), which was used directly for the next step.

LCMS (ESI, m/z): [M+1]⁺=515; RT=0.449 min & 0.573 min.

### Step 12: Synthesis of Compound 30

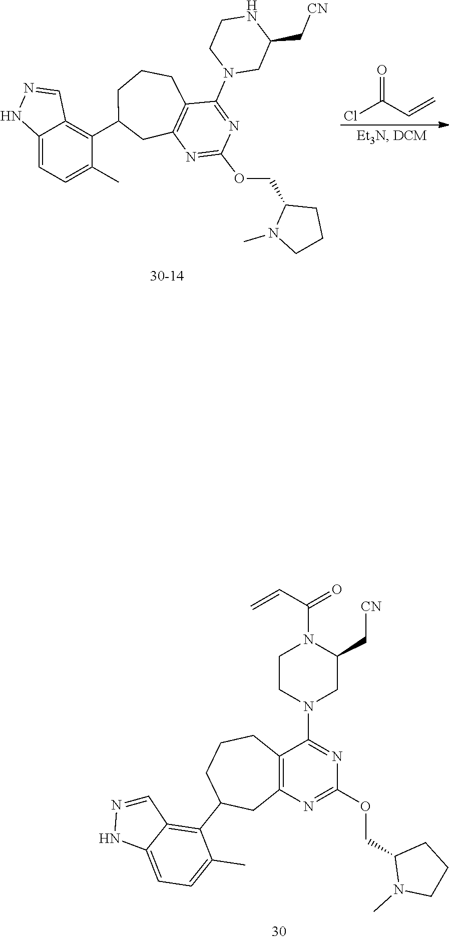

To a cooled (−10° C.) solution of 2-((2S)-4-(8-(5-methyl-1H-indazol-4-yl)-2-(((S)-1-methylpyrrolidin-2-yl)methoxy)-6,7,8,9-tetrahydro-5H-cyclohepta[d]pyrimidin-4-yl)piperazin-2-yl)acetonitrile (36 mg, 0.070 mmol, 1.0 eq.) and Et₃N (35 mg, 0.350 mmol, 5.0 eq.) in anhydrous DCM (2.5 mL) was added dropwise a solution of acryloyl chloride (8.2 mg, 0.091 mmol, 1.3 eq.) in anhydrous DCM (0.5 mL). After addition, the mixture was stirred at −10° C. for 30 min. LCMS analysis showed starting material was consumed and desired product formed. Water (10 mL) was added and the organic layer was separated. The aqueous layer was extracted with DCM (5 mL×2). The combined organics were dried over anhydrous Na₂SO₄ and concentrated. The residue was purified by prep-HPLC (ACN-H₂O+0.1% NH₄HCO₃) to obtain 2-((2S)-1-acryloyl-4-(8-(5-methyl-1H-indazol-4-yl)-2-(((S)-1-methylpyrrolidin-2-yl)methoxy)-6,7,8,9-tetrahydro-5H-cyclohepta[d]pyrimidin-4-yl)piperazin-2-yl)acetonitrile (2.30 mg, 5.7%, 30).

LCMS (ESI, m/z): [M+1]⁺=569; RT=1.506 min;

¹H NMR (400 MHz, CDCl₃) δ 10.09 (s, 1H), 8.20 (s, 1H), 7.24 (s, 1H), 7.18 (d, J=8.4 Hz, 1H), 6.59 (brs, 1H), 6.39 (d, J=16.4 Hz, 1H), 5.83 (d, J=10.0 Hz, 1H), 5.37-4.34 (m, 3H), 4.22-4.13 (m, 1H), 3.96 (brs, 0.5H), 3.80 (d, J=13.6 Hz, 1H), 3.77-3.75 (m, 0.5H), 3.74-3.69 (m, 1H), 3.64 (d, J=12.0 Hz, 1H), 3.35-3.24 (m, 2H), 3.22-3.13 (m, 2H), 3.09 (d, J=14.0 Hz, 1H), 3.05-2.88 (m, 3H), 2.80-2.68 (m, 3H), 2.50 (s, 3H), 2.38 (s, 3H), 2.35-2.28 (m, 2H), 2.27-2.19 (m 1H), 2.18-2.10 (m, 1H), 2.09-2.02 (m, 1H), 1.79-1.74 (m, 2H), 1.53-1.47 (m, 1H).

### Example 31

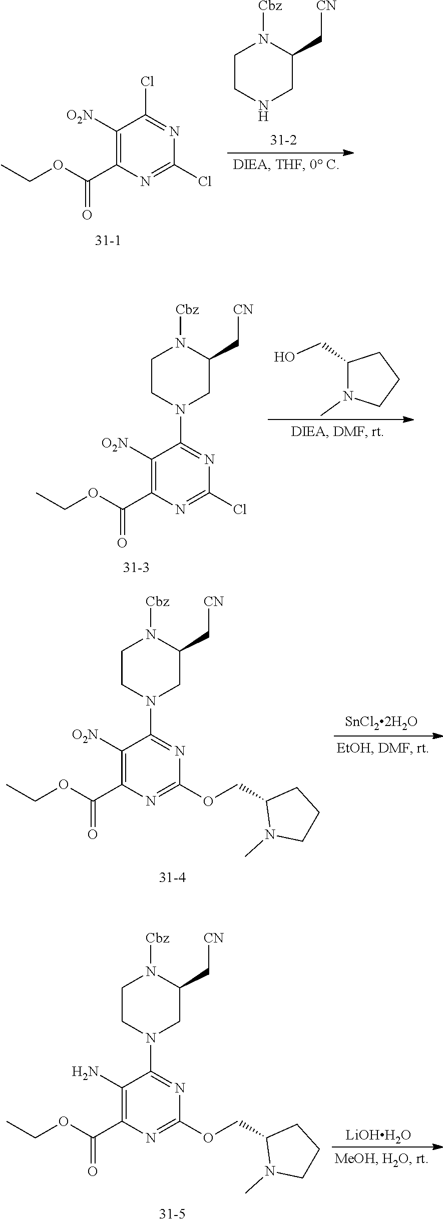

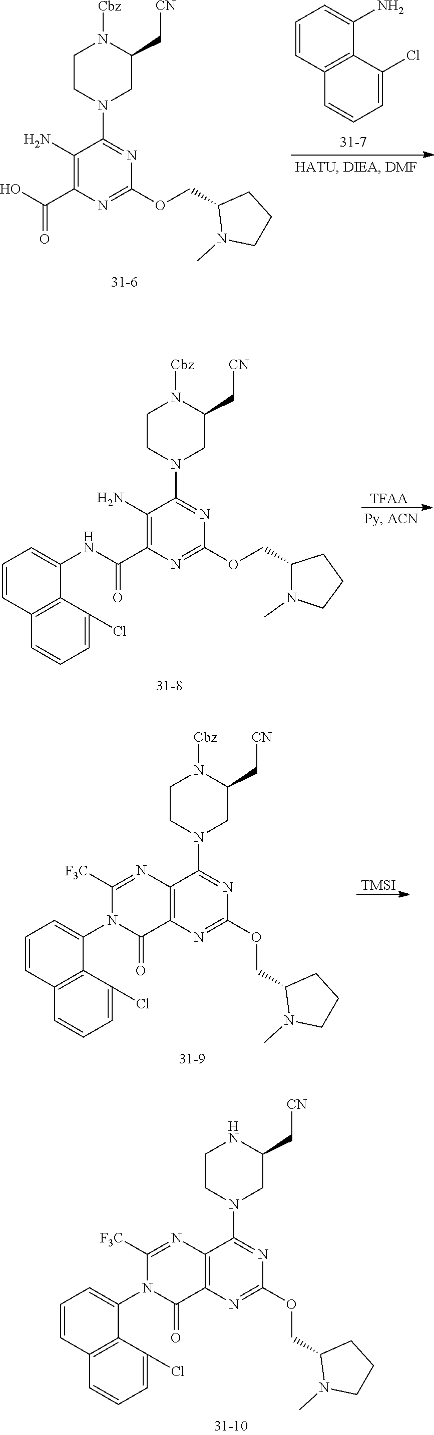

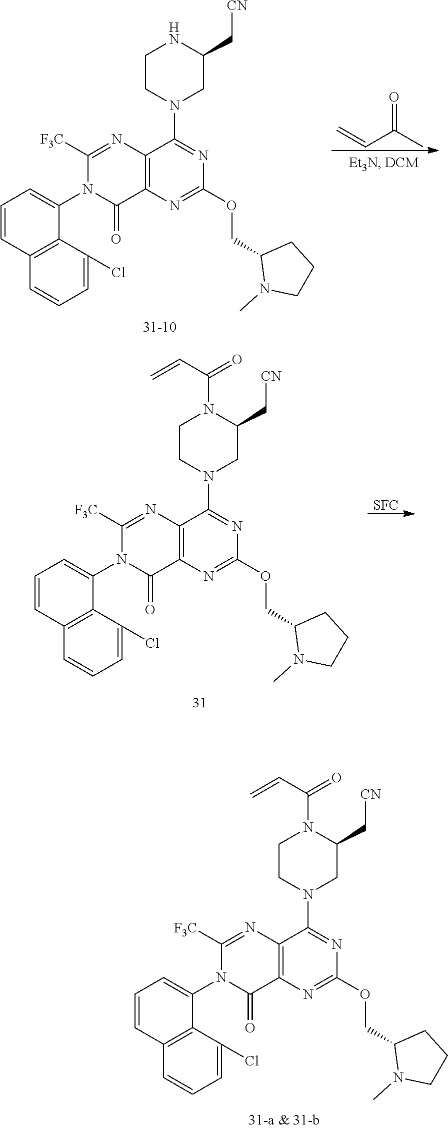

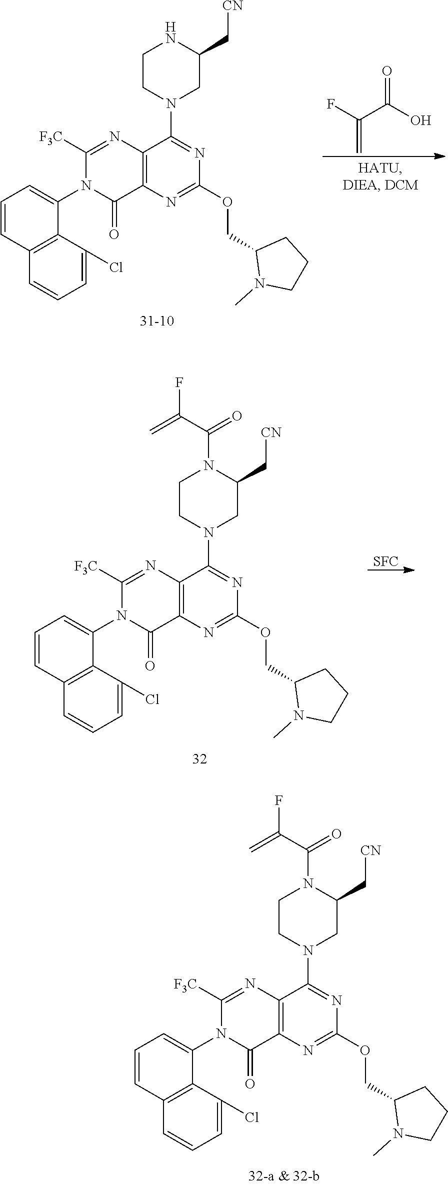

### Step 1: Synthesis of Compound 31-3

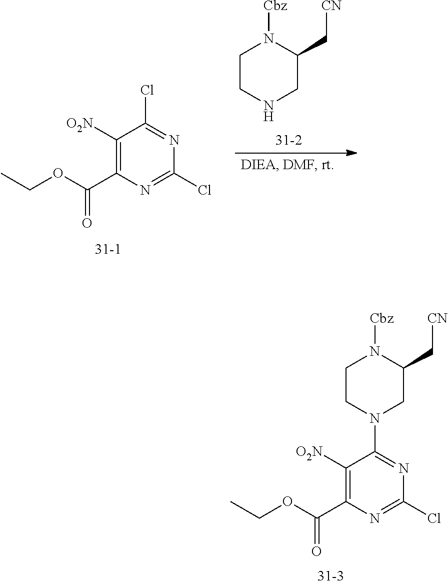 [SMILES: CCOC(=O)c1nc(Cl)nc(Cl)c1[N+](=O)[O-] ; N#CC[C@H]1CNCCN1C(=O)OCc1ccccc1 ; CCOC(=O)c1nc(Cl)nc(N2CCN(C(=O)OCc3ccccc3)[C@@H](CC#N)C2)c1[N+](=O)[O-]]

To a solution of ethyl 2,6-dichloro-5-nitropyrimidine-4-carboxylate (4.2 g, 0.016 mol, 1.0 eq.) and benzyl (S)-2-(cyanomethyl) piperazine-1-carboxylate (4.1 g, 0.016 mol, 1.0 eq.) in anhydrous THF (90 mL) was added DIEA (3.9 mL, 0.023 mol, 1.5 eq.). The reaction mixture was stirred at 0° C. under Ar for 1 h. LCMS showed starting material was consumed and desired product formed. The reaction mixture was concentrated and purified by silica column chromatography eluting with PE/EA (3:1, v/v) to obtain ethyl (S)-6-(4-((benzyloxy)carbonyl)-3-(cyanomethyl)piperazin-1-yl)-2-chloro-5-nitropyrimidine-4-carboxylate (5.4 g, 75%, 31-3).

LCMS (ESI, m/z): [M+1]⁺=489; RT=1.948 min.

### Step 2: Synthesis of Compound 31-4

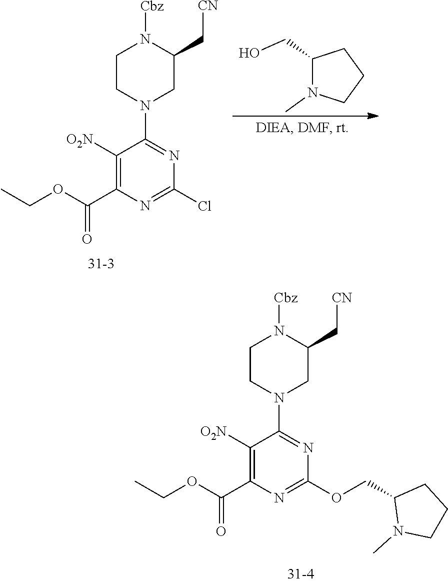

To a mixture of ethyl (S)-6-(4-((benzyloxy)carbonyl)-3-(cyanomethyl)piperazin-1-yl)-2-chloro-5-nitropyrimidine-4-carboxylate (5.4 g, 0.01 mol, 1.0 eq.) in anhydrous DMF (60.0 mL) was added (S)-(1-methylpyrrolidin-2-yl) methanol (1.9 g, 0.02 mmol, 1.5 eq.) and DIEA (3.6 ml, 0.02 mol, 2.0 eq.). The mixture was stirred at rt for 16 h. LCMS showed starting material was consumed and desired product formed. The reaction mixture was quenched with H₂O (40 mL) and extracted with EA (100 mL×3). The combined organic fractions were washed with brine (20 mL), dried over anhydrous Na₂SO₄ and concentrated. The residue was purified by prep-TLC eluting with DCM/MeOH (15:1, v/v) to obtain ethyl 6-((S)-4-((benzyloxy) carbonyl)-3-(cyanomethyl) piperazin-1-yl)-2-(((S)-1-methylpyrrolidin-2-yl) methoxy)-5-nitropyrimidine-4-carboxylate (5.4 g, 86%, 31-4).

LCMS (ESI, m/z): [M+1]⁺=568; RT=1.097 min.

### Step 3: Synthesis of Compound 31-5

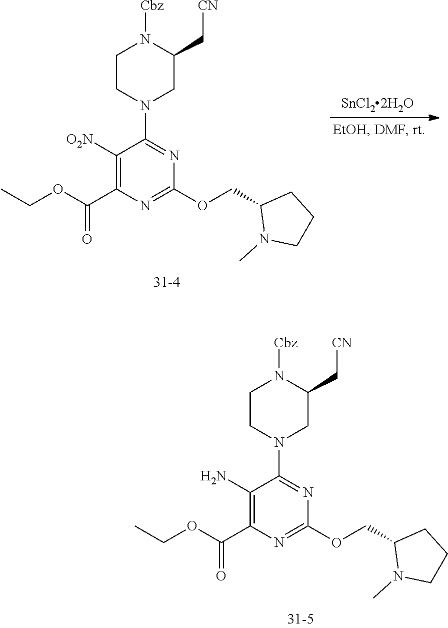 [SMILES: CCOC(=O)c1nc(OC[C@@H]2CCCN2C)nc(N2CCN(C(=O)OCc3ccccc3)[C@@H](CC#N)C2)c1[N+](=O)[O-] ; CCOC(=O)c1nc(OC[C@@H]2CCCN2C)nc(N2CCN(C(=O)OCc3ccccc3)[C@@H](CC#N)C2)c1N]

To a solution of ethyl 6-((S)-4-((benzyloxy) carbonyl)-3-(cyanomethyl) piperazin-1-yl)-2-(((S)-1-methylpyrrolidin-2-yl) methoxy)-5-nitropyrimidine-4-carboxylate (5.4 g, 0.01 mol, 1.0 eq.) in a mixture solvent of DMF (20 mL) and EtOH (60 mL), was added SnCl₂·2H₂O (10.8 g, 0.05 mol, 5.0 eq.). The reaction mixture was stirred at rt under Ar for 16 h. LCMS showed that starting material was consumed and desired product formed. The reaction mixture was concentrated to remove EtOH and then diluted with EtOAc (120 mL), followed by the addition of aq. NaHCO₃ (sat. 180 mL). The resulting mixture was filtered through celite. The organic layer of the filtrate was separated and the aqueous layer was extracted with EtOAc (160 mL×2). The combined organic fractions were washed with brine (100 mL), dried over anhydrous Na₂SO₄ and concentrated. The residue was purified by silica column chromatography eluting with DCM/MeOH (15:1, v/v) to obtain ethyl 5-amino-6-((S)-4-((benzyloxy) carbonyl)-3-(cyanomethyl) piperazin-1-yl)-2-(((S)-1-methylpyrrolidin-2-yl) methoxy) pyrimidine-4-carboxylate (2.7 g, 53%, 31-5).

LCMS (ESI, m/z): [M+1]⁺=538; RT=0.984 min.

### Step 4: Synthesis of Compound 31-6

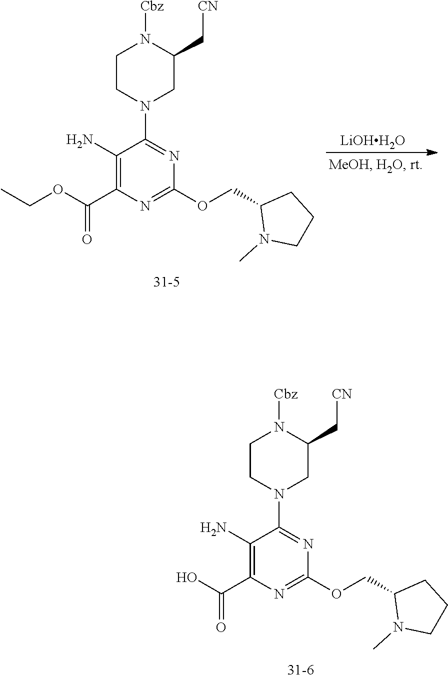 [SMILES: CCOC(=O)c1nc(OC[C@@H]2CCCN2C)nc(N2CCN(C(=O)OCc3ccccc3)[C@@H](CC#N)C2)c1N ; "LiOH·H2O" ; CN1CCC[C@H]1COc1nc(C(=O)O)c(N)c(N2CCN(C(=O)OCc3ccccc3)[C@@H](CC#N)C2)n1]

To a mixture of ethyl 5-amino-6-((S)-4-((benzyloxy) carbonyl)-3-(cyanomethyl) piperazin-1-yl)-2-(((S)-1-methylpyrrolidin-2-yl) methoxy) pyrimidine-4-carboxylate (2.7 g, 0.005 mol, 1.0 eq.) in a mixture solvent of MeOH (60 mL) and H₂O (10 mL) was added LiOH—H₂O (1.1 g, 0.025 mol, 5.0 eq.). The mixture was stirred at rt for 2 h. LCMS showed starting material was consumed and desired product formed. The reaction mixture concentrated to obtain 5-amino-6-((S)-4-((benzyloxy)carbonyl)-3-(cyanomethyl) piperazin-1-yl)-2-(((S)-1-methylpyrrolidin-2-yl)methoxy)pyrimidine-4-carboxylic acid (3.7 g, crude, 31-6).

LCMS (ESI, m/z): [M+1]⁺=510; RT=0.973 min.

### Step 5: Synthesis of Compound 31-8

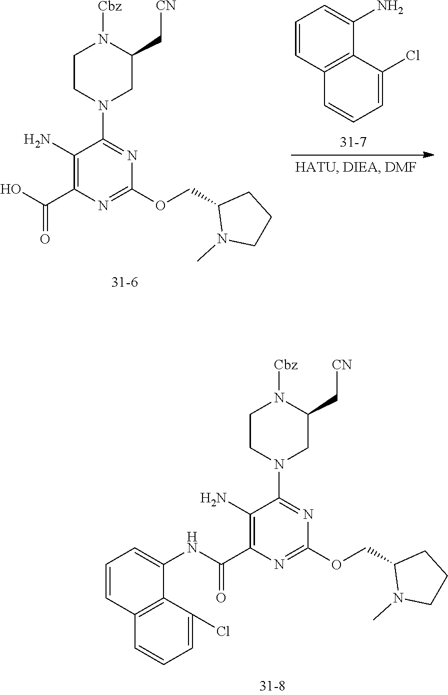 [SMILES: CN1CCC[C@H]1COc1nc(C(=O)O)c(N)c(N2CCN(C(=O)OCc3ccccc3)[C@@H](CC#N)C2)n1 ; Nc1cccc2cccc(Cl)c12 ; CN1CCC[C@H]1COc1nc(C(=O)Nc2cccc3cccc(Cl)c23)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1-yl)-2-(((S)-1-methylpyrrolidin-2-yl) methoxy) pyrimidine-4-carboxylic acid (400 mg, 0.79 mmol, 1.0 eq.) and 8-chloronaphthalen-1-amine (84 mg, 0.471 mmol, 0.6 eq.) in anhydrous DMF (4.0 mL) was added DIEA (0.4 mL, 2.36 mmol, 3.0 eq.), followed by the addition of HATU (299 mg, 0.79 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (50 mL×3). The combined organic fractions were washed with brine (20 mL), dried over anhydrous Na₂SO₄ and concentrated. The residue was purified by silica column chromatography eluting with DCM/MeOH (15:1, v/v) to obtain benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (214 mg, 41%, 31-8).

LCMS (ESI, m/z): [M+1]⁺=669.4; RT=1.255 min.

### Step 6: Synthesis of Compound 31-9

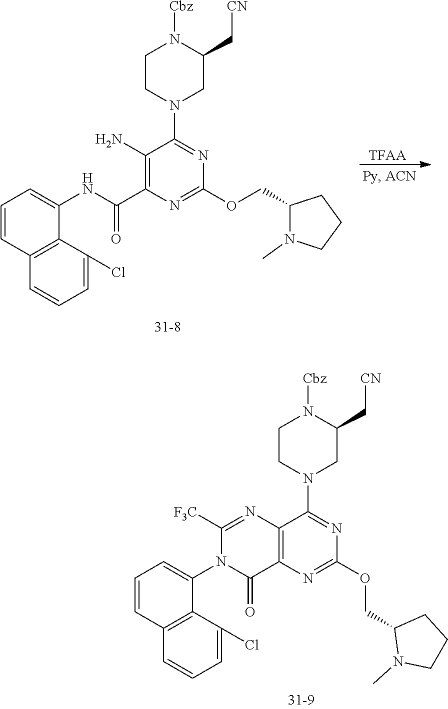 [SMILES: CN1CCC[C@H]1COc1nc(C(=O)Nc2cccc3cccc(Cl)c23)c(N)c(N2CCN(C(=O)OCc3ccccc3)[C@@H](CC#N)C2)n1 ; "TFAA" ; CN1CCC[C@H]1COc1nc(N2CCN(C(=O)OCc3ccccc3)[C@@H](CC#N)C2)c2nc(C(F)(F)F)n(-c3cccc4cccc(Cl)c34)c(=O)c2n1]

To a cooled (0° C.) solution of benzyl (S)-4-(5-amino-6-((8-chloronaphthalen-1-yl)carbamoyl)-2-(((S)-1-methylpyrrolidin-2-yl)methoxy)pyrimidin-4-yl)-2-(cyanomethyl)piperazine-1-carboxylate (189 mg, 0.282 mmol, 1.0 eq.) in anhydrous ACN (2.0 mL) was added pyridine (112 mg, 1.41 mmol, 5.0 eq.), followed by the addition of TFAA (178 mg, 0.847 mmol, 3.0 eq.). The mixture was stirred at 0° C. for 1 h and then heat to 40° C. for 2 h. LCMS showed starting material was consumed and desired product formed. The reaction mixture was cooled to room temperature and concentrated under reduced pressure. The residue was purified by prep-TLC eluting with DCM/MeOH (10:1, v/v) to obtain benzyl (S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-2-(cyanomethyl)piperazine-1-carboxylate (49 mg, 23% yield, 31-9).

LCMS (ESI, m/z): [M+1]⁺=747.1; RT=1.280 min.

### Step 7: Synthesis of Compound 31-10

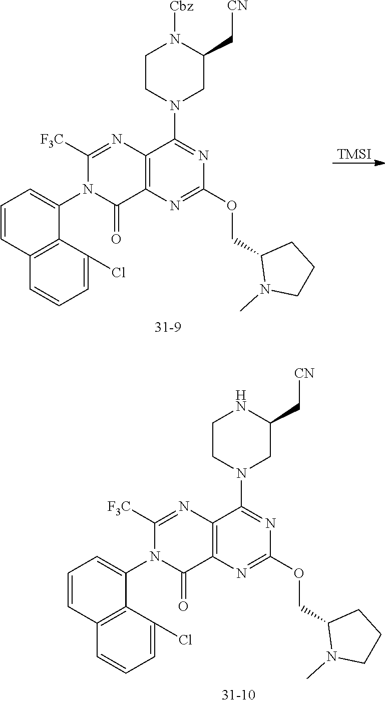 [SMILES: "TMSI" ; CN1CCC[C@H]1COc1nc(N2CCN(C(=O)OCc3ccccc3)[C@@H](CC#N)C2)c2nc(C(F)(F)F)n(-c3cccc4cccc(Cl)c34)c(=O)c2n1 ; CN1CCC[C@H]1COc1nc(N2CCN[C@@H](CC#N)C2)c2nc(C(F)(F)F)n(-c3cccc4cccc(Cl)c34)c(=O)c2n1]

To a solution of benzyl (S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-2-(cyanomethyl)piperazine-1-carboxylate (49 mg, 0.066 mmol) in anhydrous ACN (2.5 mL) was added TMSI (105 mg, 0.524 mmol), and the mixture was stirred at room temperature for 1 h. LCMS showed starting material was consumed and desired product formed. The reaction mixture was treated with Et₃N (1.0 mL) and concentrated and purified by prep-TLC eluting with DCM/MeOH (10:1, v/v) to obtain 2-((S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (28 mg, 70% yield, 31-10).

LCMS (ESI, m/z): [M+1]⁺=613.2; RT=0.805 min.

### Step 8: Synthesis of Compound 31-a & 31-b

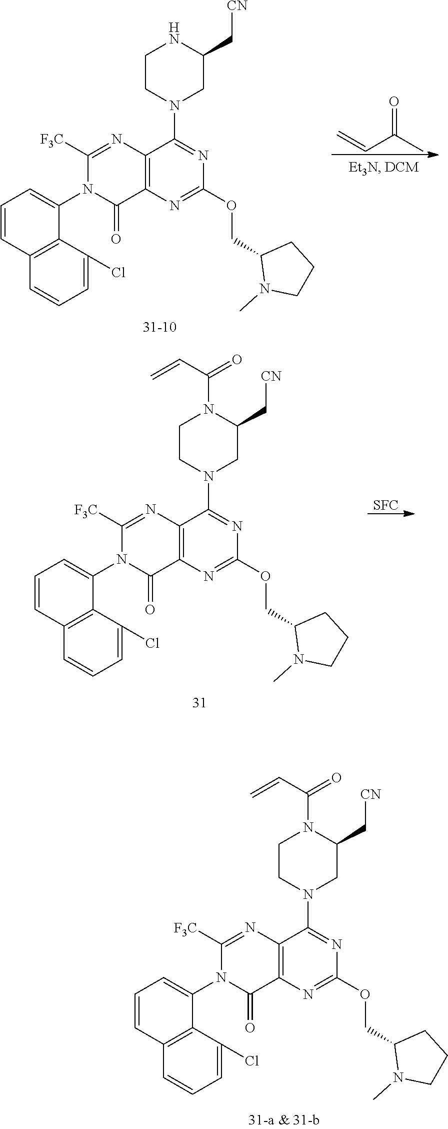

To a cooled (0° C.) solution of 2-((S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (140 mg, 0.228 mmol, 1.0 eq.) and Et₃N (115 mg, 1.14 mmol, 5.0 eq.) in DCM (2.5 mL) was added dropwise a solution of acryloyl chloride (25 mg, 0.274 mmol, 1.2 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% NH₄HCO₃) to obtain 2-((S)-1-acryloyl-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (40.45 mg, 26%, 31).

31:

LCMS (ESI, m/z): [M+1]⁺=667.1; RT=1.669 min;

¹H NMR (400 MHz, CDCl3) δ 8.08 (d, J=8.1 Hz, 1H), 7.91 (d, J=8.1 Hz, 1H), 7.63 (t, J=7.8 Hz, 1H), 7.57 (d, J=7.2 Hz, 1H), 7.45 (t, J=7.8 Hz, 2H), 6.71-6.55 (m, 1H), 6.42 (d, J=16.5 Hz, 1H), 5.85 (d, J=10.4 Hz, 1H), 5.36 (d, J=49.2 Hz, 1H), 5.09 (s, 1H), 4.54 (t, J=11.6 Hz, 1H), 4.40 (dt, J=11.1, 5.7 Hz, 1H), 4.20-3.39 (m, 4H), 3.13 (s, 1H), 2.96-2.68 (m, 3H), 2.50 (s, 3H), 2.36-2.26 (m, 1H), 2.08-1.98 (m, 1H), 1.87-1.75 (m, 4H).

¹⁹F NMR (376 MHz, CDCl3) δ −64.50, −64.78, −64.81.

Compound 31 (23 mg) was separated by SFC separation to give two products 31-a (1.76 mg) and 31-b (2.86 mg).

31-a:

LCMS (ESI, m/z): [M+1]⁺=667.2; RT=1.760 min;

31-b:

LCMS (ESI, m/z): [M+1]⁺=667.2; RT=1.750 min;

### Step 9: Synthesis of Compounds 32-a & 32-b

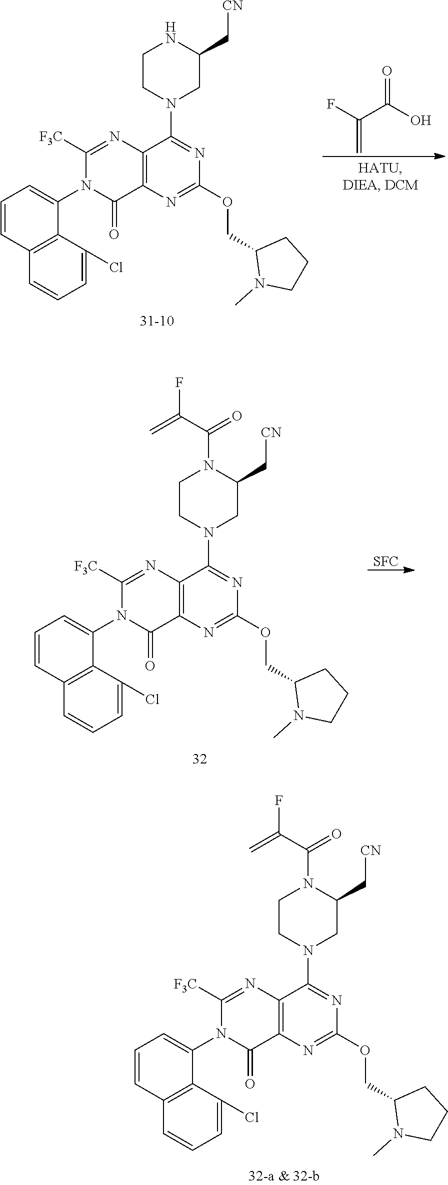

To a solution of 2-((S)-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (100 mg, 0.163 mmol, 1.0 eq.) and 2-fluoroacrylic acid (29 mg, 0.327 mmol, 2 eq.) in anhydrous DMF (4.0 mL) was added DIEA (63 mg, 0.49 mmol, 3.0 eq.), followed by the addition of HATU (124 mg, 0.327 mmol, 2.0 eq.). The reaction mixture was stirred at room temperature under Ar for 2 h. LCMS showed starting material was consumed and desired product formed. The reaction mixture was cooled to room temperature, diluted with water (15 mL) and extracted with EtOAc (15 mL×2). The combined organic fractions were washed with brine (20 mL), dried over anhydrous Na₂SO₄ and concentrated. The residue was purified by prep-HPLC to obtain 2-((S)-1-acryloyl-4-(7-(8-chloronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (22.30 mg, 20%, 32). The product was separated by SFC separation to give two products 32-a (4 mg) and 32-b (5 mg).

32-a:

LCMS_(ESI, m/z): [M+1]⁺=685.4; RT=1.030 min;

¹H NMR (400 MHz, CDCl3) δ 8.29 (s, 1H), 8.09 (d, J=7.7 Hz, 1H), 7.92 (d, J=7.7 Hz, 1H), 7.64 (t, J=7.8 Hz, 1H), 7.58 (d, J=6.8 Hz, 1H), 7.52 (d, J=7.2 Hz, 1H), 7.46 (t, J=7.8 Hz, 1H), 5.44 (d, J=48.5 Hz, 2H), 5.27 (dd, J=16.8, 3.6 Hz, 1H), 4.86 (ddd, J=14.8, 11.6, 4.0 Hz, 3H), 3.81 (t, J=133.4 Hz, 5H), 3.02-2.82 (m, 6H), 2.30-2.09 (m, 6H).

¹⁹F NMR (376 MHz, CDCl3) δ −64.83, −72.48, −74.38.

32-b:

LCMS_(ESI, m/z): [M+1]⁺=685.3; RT=1.175 min;

¹H NMR (400 MHz, CDCl3) δ 8.39 (s, 1H), 8.09 (dd, J=8.4, 1.0 Hz, 1H), 7.91 (dd, J=8.2, 0.9 Hz, 1H), 7.66-7.61 (m, 1H), 7.58 (dd, J=7.5, 1.1 Hz, 1H), 7.46 (dd, J=10.8, 4.8 Hz, 2H), 5.45 (d, J=47.8 Hz, 2H), 5.28 (dd, J=16.8, 3.7 Hz, 1H), 5.02-4.77 (m, 2H), 4.62 (dd, J=11.9, 4.4 Hz, 1H), 4.00 (d, J=155.7 Hz, 2H), 3.56 (dd, J=12.5, 5.7 Hz, 2H), 3.36-3.25 (m, 1H), 2.96 (dd, J=17.0, 7.1 Hz, 1H), 2.86 (d, J=15.6 Hz, 1H), 2.80 (s, 3H), 2.70 (dd, J=18.2, 8.2 Hz, 1H), 2.02 (dddd, J=21.8, 17.6, 15.1, 9.6 Hz, 6H).

¹⁹F NMR (376 MHz, CDCl3) δ −64.86, −72.23, −74.12.

### Example 32

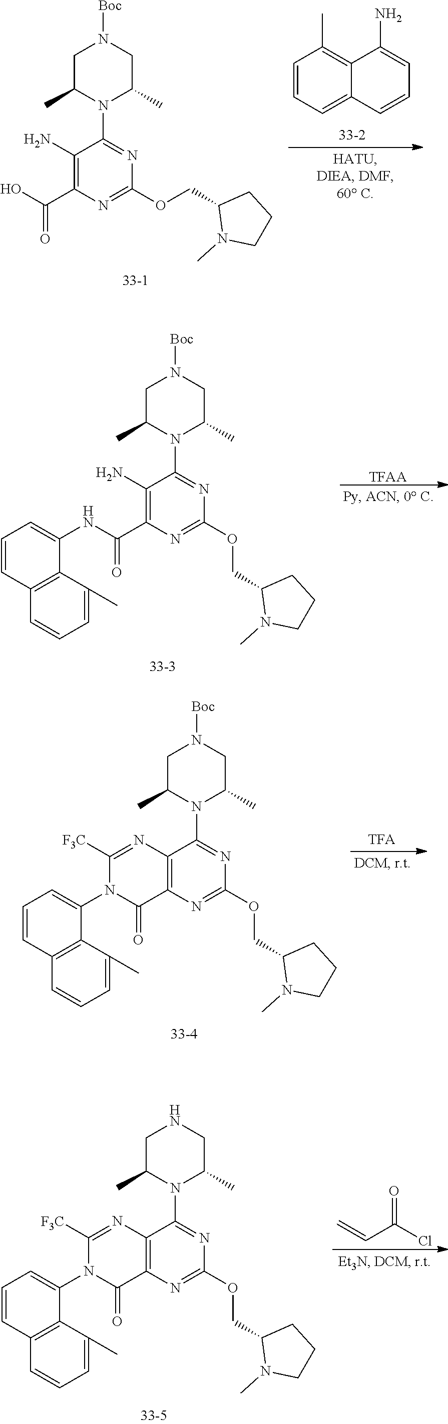

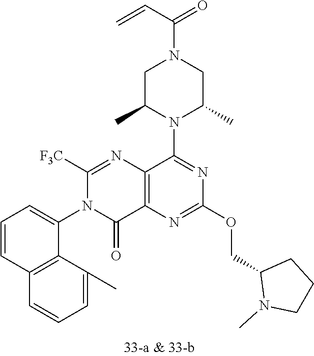 [SMILES: C=CC(=O)N1C[C@H](C)N(c2nc(OC[C@@H]3CCCN3C)nc3c(=O)n(-c4cccc5cccc(C)c45)c(C(F)(F)F)nc23)[C@@H](C)C1]

### Step 1: Synthesis of Compound 33-3

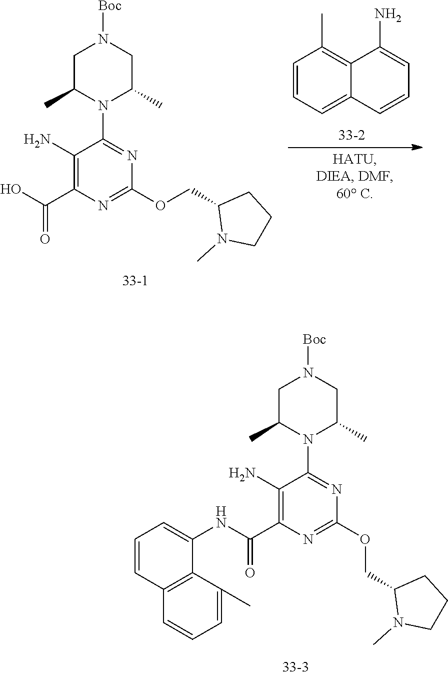 [SMILES: C[C@H]1CN(C(=O)OC(C)(C)C)C[C@H](C)N1c1nc(OC[C@@H]2CCCN2C)nc(C(=O)O)c1N ; Cc1cccc2cccc(N)c12 ; Cc1cccc2cccc(NC(=O)c3nc(OC[C@@H]4CCCN4C)nc(N4[C@@H](C)CN(C(=O)OC(C)(C)C)C[C@@H]4C)c3N)c12]

To a solution of compound 33-1 (600 mg, 1.29 mmol, 1.0 eq.) and compound 33-2 (274 mg, 1.55 mmol, 1.2 eq.) in anhydrous DMF (8 mL), was added DIEA (416 mg, 3.23 mmol, 2.5 eq.), followed by the addition of HATU (590 mg, 1.55 mmol, 1.2 eq.). The mixture was stirred at 60° C. under argon atmosphere for 3 h. LCMS showed starting material was consumed and desired product formed. The reaction mixture was cooled to room temperature, diluted with water (30 mL) and extracted with EtOAc (30 mL×3). The combined organic fractions were washed with brine (30 mL), dried over anhydrous Na₂SO₄ and concentrated. The residue was purified by silica column chromatography eluting with DCM/MeOH (10:1, v/v) to obtain tert-butyl (3S,5S)-4-(5-amino-6-((8-methylnaphthalen-1-yl)carbamoyl)-2-(((S)-1-methylpyrrolidin-2-yl)methoxy)pyrimidin-4-yl)-3,5-dimethylpiperazine-1-carboxylate (550 mg, 68% yield, 33-3).

LCMS (ESI, m/z): [M+1]⁺=624; RT=1.429 min.

### Step 2: Synthesis of Compound 33-4

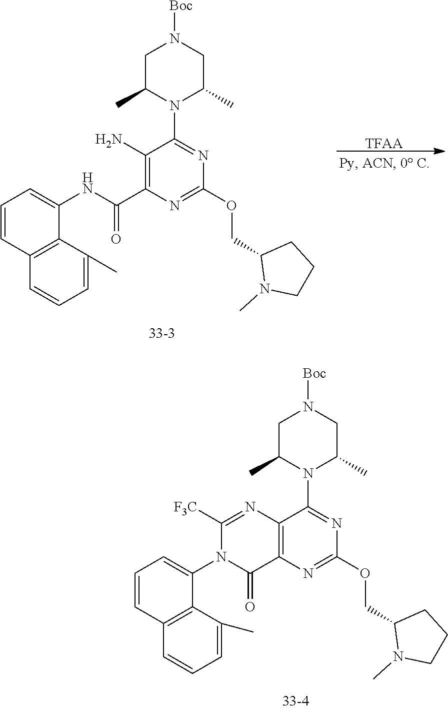 [SMILES: Cc1cccc2cccc(NC(=O)c3nc(OC[C@@H]4CCCN4C)nc(N4[C@@H](C)CN(C(=O)OC(C)(C)C)C[C@@H]4C)c3N)c12 ; "TFAA" ; Cc1cccc2cccc(-n3c(C(F)(F)F)nc4c(N5[C@@H](C)CN(C(=O)OC(C)(C)C)C[C@@H]5C)nc(OC[C@@H]5CCCN5C)nc4c3=O)c12]

To a mixture of compound 33-3 (170 mg, 0.28 mmol, 1.0 eq.) and pyridine (220 mg, 2.80 mmol, 10.0 eq.) in ACN (4 mL) at an ice/MeOH bath under argon atmosphere was added a solution of TFAA (294 mg, 1.40 mmol, 5.0 eq.) in ACN (1 mL) drop-wise. The mixture was stirred at about −5° C. for 30 min. LCMS showed starting material was consumed and desired product formed. The reaction mixture was cooled, quenched with aq. NaHCO₃ (20 mL) and extracted with EA (30 mL×3). The combined organic fractions were dried over anhydrous Na₂SO₄ and concentrated to obtain crude product of tert-butyl (3S,5S)-3,5-dimethyl-4-(7-(8-methylnaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazine-1-carboxylate (260 mg, 99% yield, 33-4), which was used directly for the next step.

LCMS (ESI, m/z): [M+1]⁺=682; RT=1.589 min.

### Step 3: Synthesis of Compound 33-5

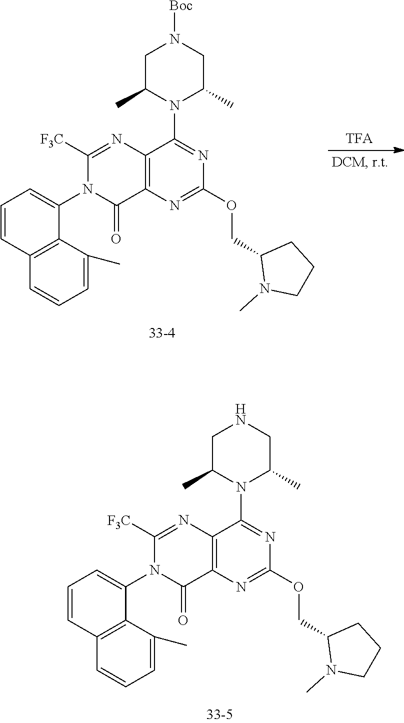 [SMILES: Cc1cccc2cccc(-n3c(C(F)(F)F)nc4c(N5[C@@H](C)CN(C(=O)OC(C)(C)C)C[C@@H]5C)nc(OC[C@@H]5CCCN5C)nc4c3=O)c12 ; Cc1cccc2cccc(-n3c(C(F)(F)F)nc4c(N5[C@@H](C)CNC[C@@H]5C)nc(OC[C@@H]5CCCN5C)nc4c3=O)c12]

To a solution of compound 33-4 (260 mg, 0.38 mmol) in DCM (5 mL) was added TFA (1 mL) at room temperature, and the mixture was stirred at room temperature overnight. LCMS showed starting material was consumed and desired product formed. The reaction mixture was concentrated and the residue was treated with aq.NaHCO3 (sat. 20 mL). The resulting mixture was extracted with DCM (20 mL×3). The combined organic fractions were dried over anhydrous Na2SO4 and concentrated to 8-((2S,6S)-2,6-dimethylpiperazin-1-yl)-3-(8-methylnaphthalen-1-yl)-6-(((S)-1-methylpyrrolidin-2-yl)methoxy)-2-(trifluoromethyl)pyrimido[5,4-d]pyrimidin-4(3H)-one (180 mg, 81% yield, 33-5), which was used directly for the next step.

LCMS (ESI, m/z): [M+1]⁺=582; RT=1.147 min.

### Step 4: Synthesis of Compounds 33-a and 33-b

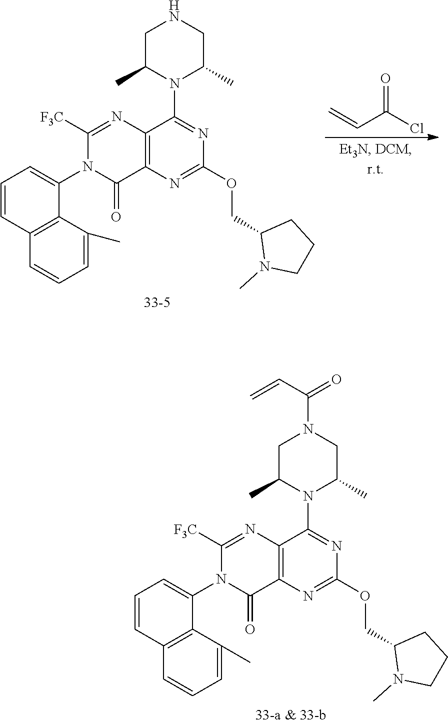

To a cooled (0° C.) solution of compound 33-5 (180 mg, 0.31 mmol, 1.0 eq.) and Et₃N (94 mg, 0.93 mmol, 3.0 eq.) in anhydrous DCM (4 mL) was added dropwise a solution of acryloyl chloride (41 mg, 0.46 mmol, 1.5 eq.) in anhydrous DCM (2 mL). After addition, the mixture was stirred at 0° C. for 30 min. LCMS showed starting material was consumed and desired product formed. Water (20 mL) was added and the organic layer was separated. The aqueous layer was extracted with DCM (20 mL×3). The combined organic fractions were dried over anhydrous Na₂SO₄ and concentrated. The residue was purified by prep-HPLC (ACN-H₂O+0.1% HCOOH) and then SFC to obtain 33-a (2.4 mg, 1% yield) and 33-b (18.5 mg, 9% yield).

33-a:

LCMS (ESI, m/z): [M+1]⁺=636; RT=1.309 min;

¹H NMR (400 MHz, DMSO) δ 8.08 (d, J=76.6 Hz, 2H), 7.70 (d, J=33.6 Hz, 2H), 7.45 (d, J=37.3 Hz, 2H), 6.79 (s, 1H), 6.23 (d, J=15.5 Hz, 1H), 5.78 (s, 1H), 5.33 (s, 1H), 4.31 (d, J=65.3 Hz, 2H), 4.03 (s, 3H), 3.66 (d, J=11.0 Hz, 2H), 2.96 (s, 1H), 2.59 (s, 1H), 2.36 (s, 3H), 2.23 (s, 4H), 1.96 (s, 1H), 1.67 (s, 3H), 1.43 (s, 6H).

¹⁹F NMR (400 MHz, DMSO) δ −63.57.

33-b:

LCMS (ESI, m/z): [M+1]⁺=636; RT=1.316 min;

¹H NMR (400 MHz, DMSO) δ 8.17 (dd, J=8.0, 1.5 Hz, 1H), 7.97 (d, J=8.0 Hz, 1H), 7.72-7.62 (m, 2H), 7.53-7.46 (m, 1H), 7.39 (d, J=7.0 Hz, 1H), 6.79 (dd, J=16.7, 10.4 Hz, 1H), 6.22 (dd, J=16.7, 2.3 Hz, 1H), 5.77 (dd, J=10.4, 2.2 Hz, 1H), 5.32 (s, 1H), 4.39 (dd, J=10.8, 5.0 Hz, 1H), 4.21 (dd, J=10.8, 6.2 Hz, 1H), 4.02 (t, J=15.5 Hz, 3H), 3.65 (dd, J=14.4, 3.6 Hz, 1H), 2.99-2.93 (m, 1H), 2.62 (dd, J=14.0, 5.9 Hz, 1H), 2.36 (s, 3H), 2.20 (s, 3H), 2.00-1.91 (m, 1H), 1.72-1.59 (m, 3H), 1.40 (dd, J=6.4, 4.2 Hz, 6H).

¹⁹F NMR (400 MHz, DMSO) δ −63.53.

### Example 33

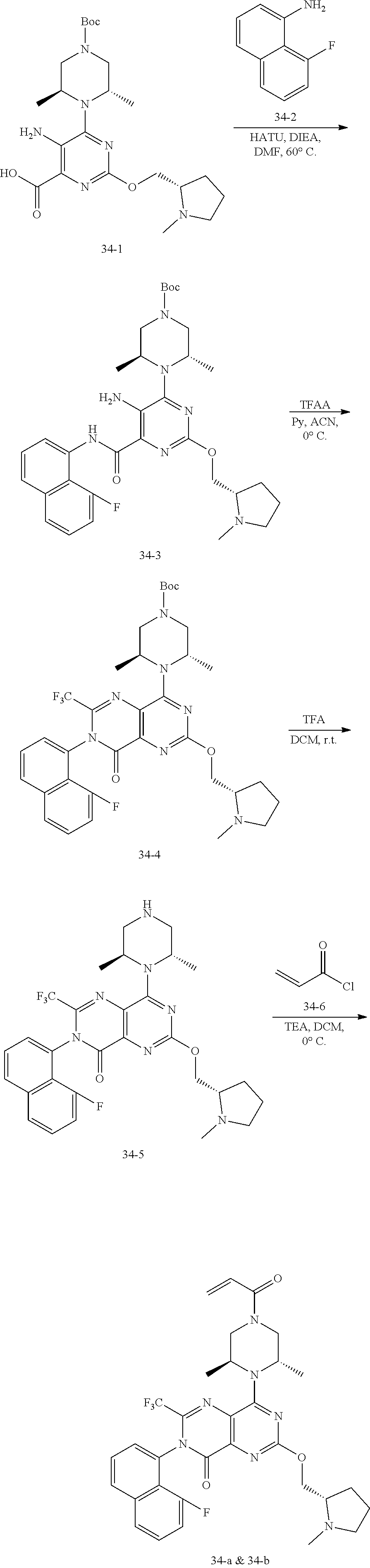

### Step 1: Synthesis of Compound 34-3

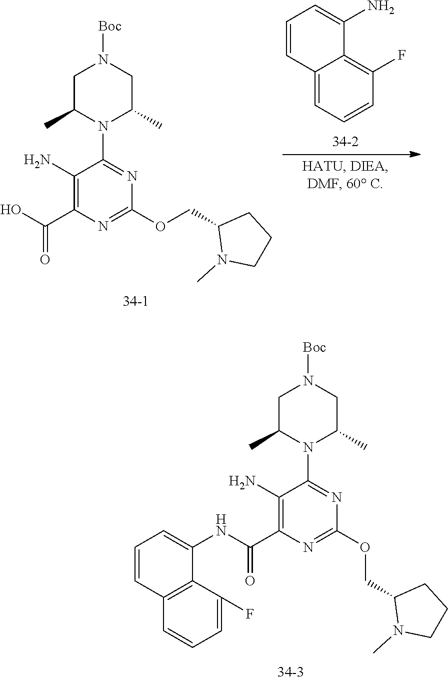 [SMILES: C[C@H]1CN(C(=O)OC(C)(C)C)C[C@H](C)N1c1nc(OC[C@@H]2CCCN2C)nc(C(=O)O)c1N ; Nc1cccc2cccc(F)c12 ; C[C@H]1CN(C(=O)OC(C)(C)C)C[C@H](C)N1c1nc(OC[C@@H]2CCCN2C)nc(C(=O)Nc2cccc3cccc(F)c23)c1N]

To a mixture of 5-amino-6-((2S,6S)-4-(tert-butoxycarbonyl)-2,6-dimethylpiperazin-1-yl)-2-(((S)-1-methylpyrrolidin-2-yl) methoxy) pyrimidine-4-carboxylic acid (250 mg, 0.54 mmol, 1.0 eq.) and 8-fluoronaphthalen-1-amine (69 mg, 0.43 mmol, 0.8 eq.) in anhydrous DMF (10 mL), was added DIEA (209 mg, 1.62 mmol, 3.0 eq.), followed by the addition of HATU (208 mg, 0.54 mmol, 1.0 eq.). The mixture was stirred at 60° C. under Ar for 2 h. LCMS showed starting material was consumed and desired product formed. The reaction mixture was cooled to room temperature, diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with DCM/MeOH (15:1, v/v) to obtain tert-butyl (3S,5S)-4-(5-amino-6-((8-fluoronaphthalen-1-yl)carbamoyl)-2-(((S)-1-methylpyrrolidin-2-yl)methoxy)pyrimidin-4-yl)-3,5-dimethylpiperazine-1-carboxylate (230 mg, 70% yield, 34-3).

LCMS (ESI, m/z): [M+1]⁺=609; RT=1.138 min.

### Step 2: Synthesis of Compound 34-4

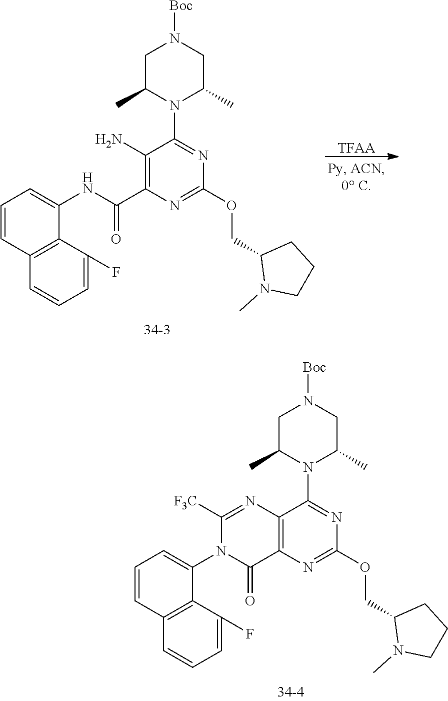 [SMILES: C[C@H]1CN(C(=O)OC(C)(C)C)C[C@H](C)N1c1nc(OC[C@@H]2CCCN2C)nc(C(=O)Nc2cccc3cccc(F)c23)c1N ; "TFAA" ; C[C@H]1CN(C(=O)OC(C)(C)C)C[C@H](C)N1c1nc(OC[C@@H]2CCCN2C)nc2c(=O)n(-c3cccc4cccc(F)c34)c(C(F)(F)F)nc12]

To a cooled (0° C.) solution of tert-butyl (3S,5S)-4-(5-amino-6-((8-fluoronaphthalen-1-yl)carbamoyl)-2-(((S)-1-methylpyrrolidin-2-yl)methoxy)pyrimidin-4-yl)-3,5-dimethylpiperazine-1-carboxylate (230 mg, 0.38 mmol, 1.0 eq.) in anhydrous ACN (5.0 mL) was added pyridine (79 mg, 3.8 mmol, 10.0 eq.), followed by the addition of TFAA (477 mg, 2.28 mmol, 6.0 eq.). The mixture was stirred at 0° C. for 30 min. LCMS showed starting material was consumed and desired product formed. The reaction mixture was quenched with aq. NH₄Cl (sat. 25 mL) and extracted with EtOAc (15 mL×3). The combined organic fractions were washed with brine (20 mL), dried over anhydrous Na₂SO₄ and concentrated. The residue was purified by prep-TLC eluting with DCM/MeOH (10:1, v/v) to obtain tert-butyl (3S,5S)-4-(7-(8-fluoronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-3,5-dimethylpiperazine-1-carboxylate (200 mg, crude, 34-4).

LCMS (ESI, m/z): [M+1]⁺=686; RT=0.973 min.

### Step 3: Synthesis of Compound 34-5

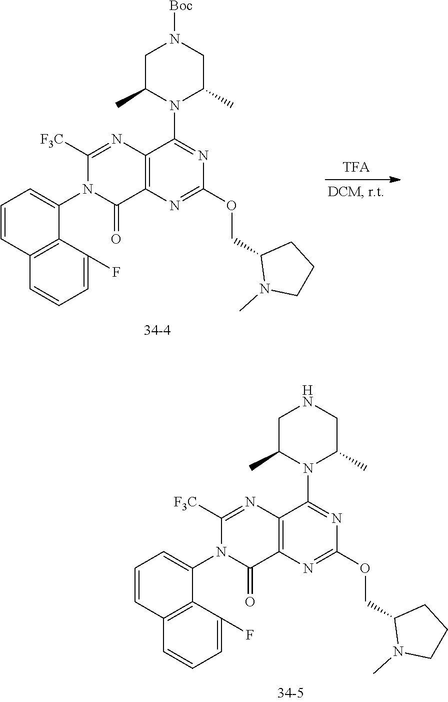 [SMILES: C[C@H]1CN(C(=O)OC(C)(C)C)C[C@H](C)N1c1nc(OC[C@@H]2CCCN2C)nc2c(=O)n(-c3cccc4cccc(F)c34)c(C(F)(F)F)nc12 ; C[C@H]1CNC[C@H](C)N1c1nc(OC[C@@H]2CCCN2C)nc2c(=O)n(-c3cccc4cccc(F)c34)c(C(F)(F)F)nc12]

To a solution of tert-butyl (3S,5S)-4-(7-(8-fluoronaphthalen-1-yl)-2-(((S)-1-methylpyrrolidin-2-yl)methoxy)-8-oxo-6-(trifluoromethyl)-7,8-dihydropyrimido, [5,4-d]pyrimidin-4-yl)-3,5-dimethylpiperazine-1-carboxylate (200 mg, 0.29 mmol) in DCM (6.0 mL) was added TFA (3.0 mL), and the mixture was stirred at room temperature for 1 h. LCMS showed starting material was consumed and desired product formed. The resulting mixture was concentrated to obtain 8-((2S,6S)-2,6-dimethylpiperazin-1-yl)-3-(8-fluoronaphthalen-1-yl)-6-(((S)-1-methylpyrrolidin-2-yl)methoxy)-2-(trifluoromethyl)pyrimido[5,4-d]pyrimidin-4(3H)-one (TFA salt, 120 mg, crude, 34-5), which was used directly for the next step.

LCMS (ESI, m/z): [M+1]⁺=586; RT=0.997 min.

### Step 4: Synthesis of Compounds 34-a and 34-b

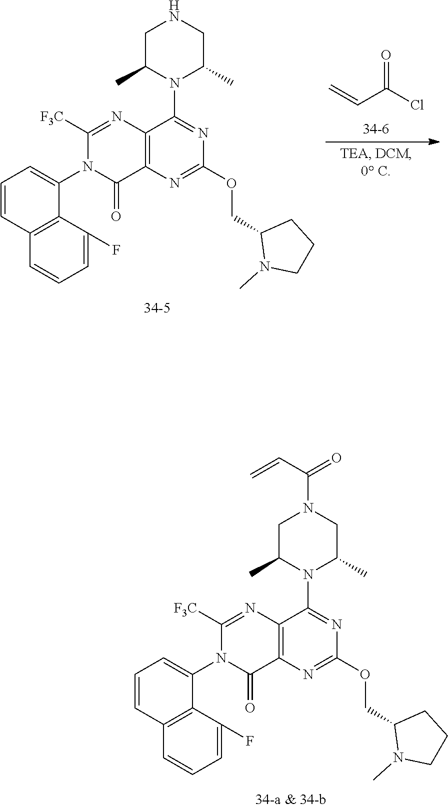

To a cooled (0° C.) solution of 3-(8-chloronaphthalen-1-yl)-8-((2S,6S)-2,6-dimethylpiperazin-1-yl)-6-(((S)-1-methylpyrrolidin-2-yl) methoxy)-2-(trifluoromethyl) pyrimido [5,4-d] pyrimidin-4(3H)-one (120 mg, 0.21 mmol, 1.0 eq.) and Et₃N (64 mg, 0.63 mmol, 3.0 eq.) in DCM (3 mL) was added drop-wise a solution of acryloyl chloride (28 mg, 0.32 mmol, 1.5 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((2S,6S)-4-acryloyl-2,6-dimethylpiperazin-1-yl)-3-(8-fluoronaphthalen-1-yl)-6-(((S)-1-methylpyrrolidin-2-yl)methoxy)-2-(trifluoromethyl)pyrimido[5,4-d]pyrimidin-4(3H)-one (4.5 mg, 1.75% yield, 34-a), and 8-((2S,6S)-4-acryloyl-2,6-dimethylpiperazin-1-yl)-3-(8-fluoronaphthalen-1-yl)-6-(((S)-1-methylpyrrolidin-2-yl)methoxy)-2-(trifluoromethyl)pyrimido[5,4-d]pyrimidin-4(3H)-one (5.6 mg, 6.17% yield, 34-b).

34-a:

LCMS (ESI, m/z): [M+1]⁺=640; RT=1.288 min;

¹H NMR (400 MHz, CDCl3) δ 8.05 (d, J=8.3 Hz, 1H), 7.76 (d, J=8.2 Hz, 1H), 7.63 (t, J=7.9 Hz, 1H), 7.45 (ddd, J=17.4, 10.2, 6.1 Hz, 2H), 7.13 (dd, J=13.2, 7.7 Hz, 1H), 6.59 (dd, J=16.7, 10.3 Hz, 1H), 6.45 (dd, J=16.7, 1.9 Hz, 1H), 5.81 (dd, J=10.3, 1.8 Hz, 1H), 5.35 (s, 1H), 4.54 (d, J=50.2 Hz, 2H), 4.19 (d, J=13.4 Hz, 1H), 4.08 (dd, J=12.6, 3.4 Hz, 1H), 3.86-3.72 (m, 2H), 3.29 (s, 1H), 3.00 (s, 1H), 2.64 (s, 3H), 2.48 (s, 1H), 2.14 (s, 1H), 1.86 (s, 4H), 1.49 (dd, J=6.4, 2.0 Hz, 6H).

¹⁹F NMR (400 MHz, CDCl₃) δ −64.82, 121.79.

34-b:

LCMS (ESI, m/z): [M+1]⁺=640; RT=1.293 min;

¹H NMR (400 MHz, CDCl3) δ 8.07 (d, J=9.3 Hz, 1H), 7.77 (d, J=8.0 Hz, 1H), 7.70-7.61 (m, 1H), 7.48 (q, J=7.8 Hz, 2H), 7.15 (dd, J=13.3, 7.1 Hz, 1H), 6.59 (dd, J=16.8, 10.3 Hz, 1H), 6.45 (dd, J=16.8, 2.0 Hz, 1H), 5.81 (dd, J=10.3, 2.0 Hz, 1H), 5.35 (d, J=5.9 Hz, 1H), 4.94 (s, 1H), 4.74 (d, J=9.1 Hz, 1H), 4.20 (d, J=13.1 Hz, 1H), 4.09 (dd, J=12.8, 3.5 Hz, 1H), 3.84 (d, J=12.1 Hz, 1H), 3.74 (dt, J=11.8, 5.9 Hz, 3H), 2.98 (s, 3H), 2.34 (dd, J=14.0, 9.6 Hz, 1H), 2.29-1.90 (m, 5H), 1.51 (d, J=6.5 Hz, 6H).

¹⁹F NMR (400 MHz, CDCl₃) δ −64.92, 121.71.

### Example 34

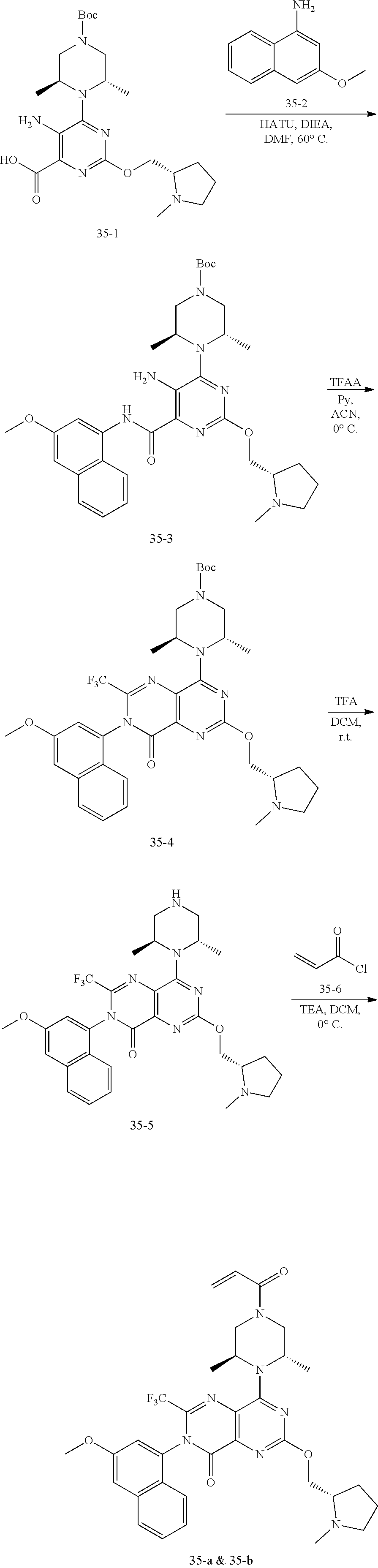

### Step 1: Synthesis of Compound 35-3

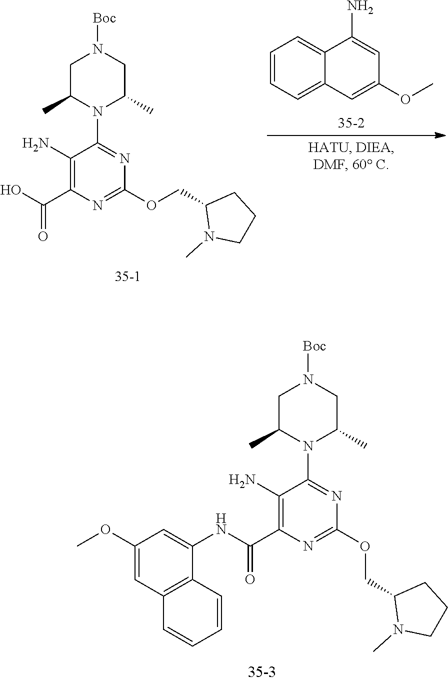 [SMILES: C[C@H]1CN(C(=O)OC(C)(C)C)C[C@H](C)N1c1nc(OC[C@@H]2CCCN2C)nc(C(=O)O)c1N ; COc1cc(N)c2ccccc2c1 ; COc1cc(NC(=O)c2nc(OC[C@@H]3CCCN3C)nc(N3[C@@H](C)CN(C(=O)OC(C)(C)C)C[C@@H]3C)c2N)c2ccccc2c1]

To a mixture of 5-amino-6-((2S,6S)-4-(tert-butoxycarbonyl)-2,6-dimethylpiperazin-1-yl)-2-(((S)-1-methylpyrrolidin-2-yl) methoxy) pyrimidine-4-carboxylic acid (300 mg, 0.65 mmol, 1.0 eq.) and 3-methoxynaphthalen-1-amine (112 mg, 0.65 mmol, 1.0 eq.) in anhydrous DMF (10 mL), was added DIEA (252 mg, 1.95 mmol, 3.0 eq.), followed by the addition of HATU (250 mg, 0.65 mmol, 1.0 eq.). The mixture was stirred at 60° C. under Ar for 2 h. LCMS showed starting material was consumed and desired product formed. The reaction mixture was cooled to room temperature, diluted with water (50 mL) and extracted with EtOAc (30 mL×3). The combined organic fractions were washed with brine (50 mL), dried over anhydrous Na₂SO₄ and concentrated. The residue was purified by silica column chromatography eluting with DCM/MeOH (15:1, v/v) to obtain tert-butyl (3 S,5S)-4-(5-amino-6-((3-methoxynaphthalen-1-yl)carbamoyl)-2-(((S)-1-methylpyrrolidin-2-yl)methoxy)pyrimidin-4-yl)-3,5-dimethylpiperazine-1-carboxylate (210 mg, 52.5% yield, 35-3).

LCMS (ESI, m/z): [M+1]⁺=620; RT=1.189 min.

### Step 2: Synthesis of Compound 35-4

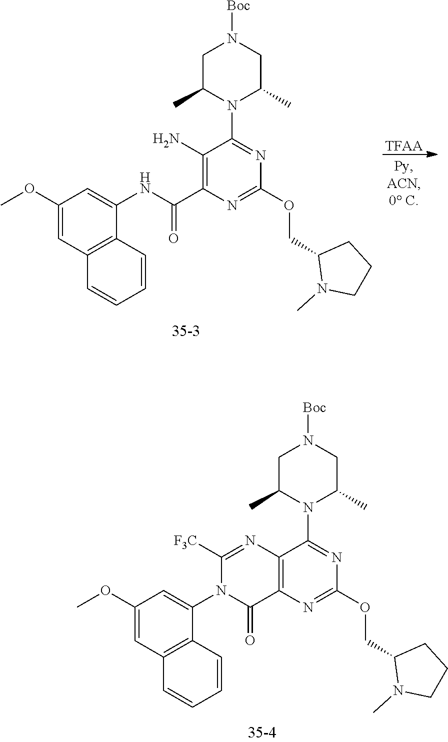 [SMILES: COc1cc(NC(=O)c2nc(OC[C@@H]3CCCN3C)nc(N3[C@@H](C)CN(C(=O)OC(C)(C)C)C[C@@H]3C)c2N)c2ccccc2c1 ; "TFAA" ; COc1cc(-n2c(C(F)(F)F)nc3c(N4[C@@H](C)CN(C(=O)OC(C)(C)C)C[C@@H]4C)nc(OC[C@@H]4CCCN4C)nc3c2=O)c2ccccc2c1]

To a cooled (0° C.) solution of tert-butyl (3S,5S)-4-(5-amino-6-((3-methoxynaphthalen-1-yl)carbamoyl)-2-(((S)-1-methylpyrrolidin-2-yl)methoxy)pyrimidin-4-yl)-3,5-dimethylpiperazine-1-carboxylate (190 mg, 0.31 mmol, 1.0 eq.) in anhydrous ACN (5.0 mL) was added pyridine (245 mg, 3.10 mmol, 10.0 eq.), followed by the addition of TFAA (387 mg, 1.86 mmol, 6.0 eq.). The mixture was stirred at 0° C. for 30 min. LCMS showed starting material was consumed and desired product formed. The reaction mixture was quenched with aq. NH₄Cl (sat. 25 mL) and extracted with EtOAc (15 mL×3). The combined organic fractions were washed with brine (20 mL), dried over anhydrous Na₂SO₄ and concentrated. The residue was purified by prep-TLC eluting with DCM/MeOH (10:1, v/v) to obtain tert-butyl (3S,5S)-4-(7-(3-methoxynaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-3,5-dimethylpiperazine-1-carboxylate (200 mg, crude, 35-4).

LCMS (ESI, m/z): [M+1]⁺=698; RT=1.218 min.

### Step 3: Synthesis of Compound 35-5

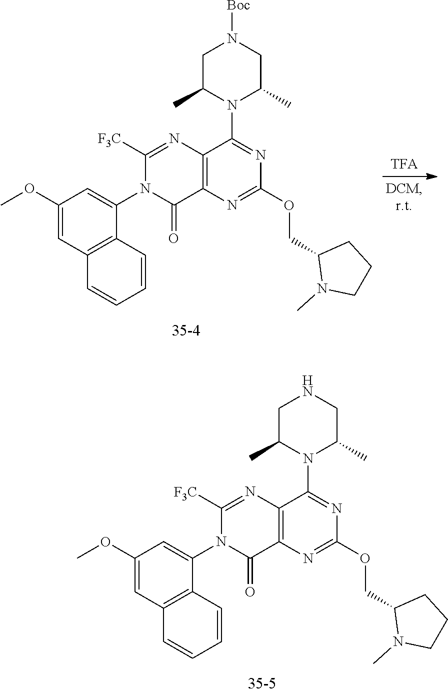 [SMILES: COc1cc(-n2c(C(F)(F)F)nc3c(N4[C@@H](C)CN(C(=O)OC(C)(C)C)C[C@@H]4C)nc(OC[C@@H]4CCCN4C)nc3c2=O)c2ccccc2c1 ; COc1cc(-n2c(C(F)(F)F)nc3c(N4[C@@H](C)CNC[C@@H]4C)nc(OC[C@@H]4CCCN4C)nc3c2=O)c2ccccc2c1]

To a solution of tert-butyl (3S,5S)-4-(7-(3-methoxynaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)-3,5-dimethylpiperazine-1-carboxylate (200 mg, 0.29 mmol) in DCM (6.0 mL) was added TFA (3.0 mL), and the mixture was stirred at room temperature for 1 h. LCMS showed starting material was consumed and desired product formed. The resulting mixture was concentrated to obtain 8-((2S,6S)-2,6-dimethylpiperazin-1-yl)-3-(3-methoxynaphthalen-1-yl)-6-(((S)-1-methylpyrrolidin-2-yl)methoxy)-2-(trifluoromethyl)pyrimido[5,4-d]pyrimidin-4(3H)-one (TFA salt, 120 mg, crude, 35-5), which was used directly for the next step.

LCMS (ESI, m/z): [M+1]⁺=598; RT=0.833 min.

### Step 4: Synthesis of Compounds 35-a and 35-b

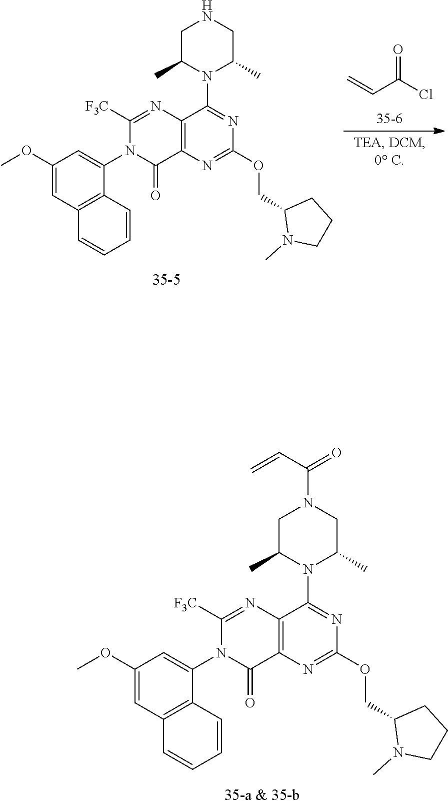

To a cooled (0° C.) solution of 3-(8-chloronaphthalen-1-yl)-8-((2S,6S)-2,6-dimethylpiperazin-1-yl)-6-(((S)-1-methylpyrrolidin-2-yl) methoxy)-2-(trifluoromethyl) pyrimido [5,4-d] pyrimidin-4(3H)-one (150 mg, 0.25 mmol, 1.0 eq.) and Et₃N (78 mg, 0.75 mmol, 3.0 eq.) in DCM (3 mL) was added drop-wise a solution of acryloyl chloride (35 mg, 0.38 mmol, 1.5 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) and then SFC to obtain 8-((2S,6S)-4-acryloyl-2,6-dimethylpiperazin-1-yl)-3-(3-methoxynaphthalen-1-yl)-6-(((S)-1-methylpyrrolidin-2-yl)methoxy)-2-(trifluoromethyl)pyrimido[5,4-d]pyrimidin-4(3H)-one (24.51 mg, 14.98% yield, 35-a), and 8-((2S,6S)-4-acryloyl-2,6-dimethylpiperazin-1-yl)-3-(3-methoxynaphthalen-1-yl)-6-(((S)-1-methylpyrrolidin-2-yl)methoxy)-2-(trifluoromethyl)pyrimido[5,4-d]pyrimidin-4(3H)-one (35.44 mg, 21.67% yield, 35-b).

35-a:

LCMS (ESI, m/z): [M+1]⁺=652; RT=1.306 min;

¹H NMR (400 MHz, CDCl3) δ 7.83 (d, J=8.3 Hz, 1H), 7.49 (ddd, J=8.2, 6.3, 1.7 Hz, 1H), 7.36-7.28 (m, 3H), 7.12 (s, 1H), 6.59 (dd, J=16.8, 10.3 Hz, 1H), 6.45 (dd, J=16.8, 2.0 Hz, 1H), 5.81 (dd, J=10.3, 2.0 Hz, 1H), 5.50 (s, 1H), 4.44 (d, J=49.5 Hz, 2H), 4.20 (d, J=14.4 Hz, 1H), 4.08 (dd, J=12.8, 3.5 Hz, 1H), 3.96 (s, 3H), 3.86-3.69 (m, 2H), 3.15 (s, 1H), 2.78 (s, 1H), 2.53 (s, 3H), 2.33 (s, 1H), 2.09 (s, 1H), 1.79 (s, 4H), 1.53-1.45 (m, 6H).

¹⁹F NMR (400 MHz, CDCl₃) δ −64.20.

35-b:

LCMS (ESI, m/z): [M+1]⁺=652; RT=1.312 min;

¹H NMR (400 MHz, CDCl3) δ 7.83 (d, J=8.3 Hz, 1H), 7.49 (t, J=6.8 Hz, 1H), 7.35-7.27 (m, 3H), 7.20 (s, 1H), 6.59 (dd, J=16.8, 10.3 Hz, 1H), 6.45 (dd, J=16.7, 1.9 Hz, 1H), 5.81 (dd, J=10.3, 1.9 Hz, 1H), 5.46 (s, 1H), 4.60 (d, J=90.6 Hz, 2H), 4.18 (t, J=14.1 Hz, 1H), 4.08 (dd, J=12.8, 3.3 Hz, 1H), 3.97 (s, 3H), 3.86-3.72 (m, 2H), 3.34 (s, 1H), 3.08 (s, 1H), 2.60 (d, J=62.2 Hz, 4H), 2.38-1.56 (m, 5H), 1.50 (d, J=5.4 Hz, 6H).

¹⁹F NMR (400 MHz, CDCl₃) δ −64.44.

### Example 35

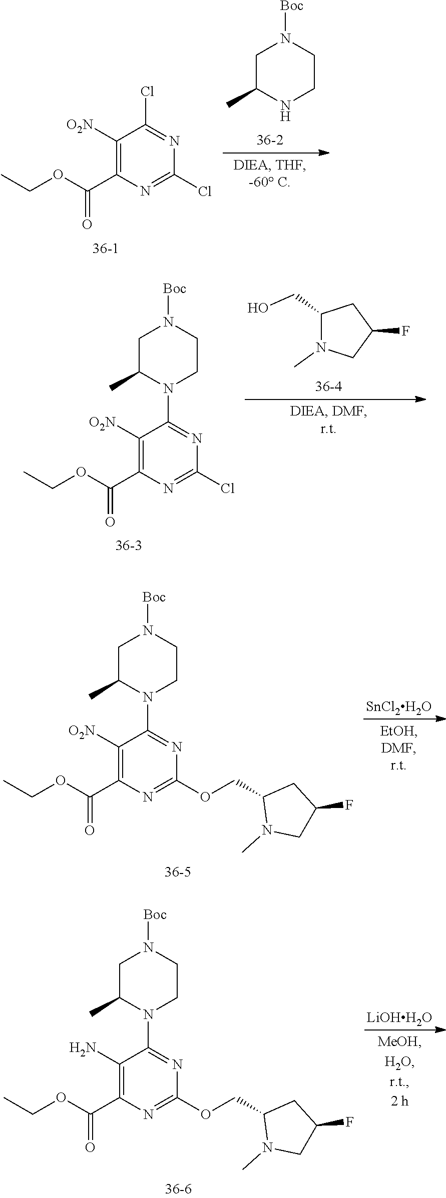

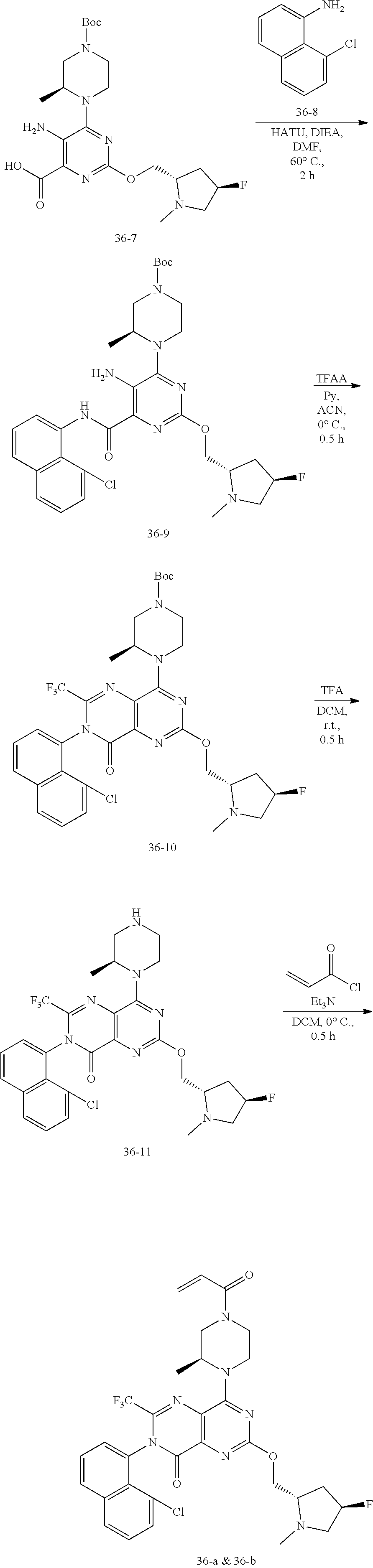

### Step 1: Synthesis of Compound 36-3

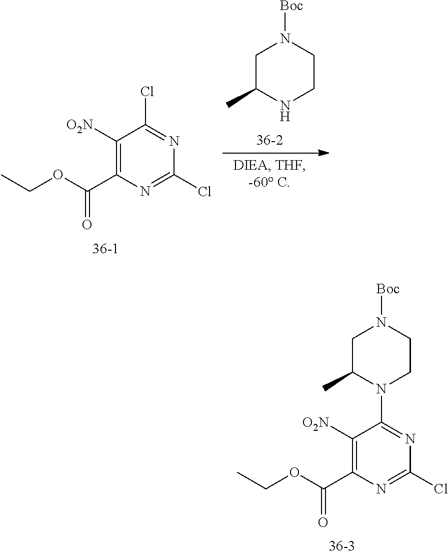 [SMILES: CCOC(=O)c1nc(Cl)nc(Cl)c1[N+](=O)[O-] ; C[C@H]1CN(C(=O)OC(C)(C)C)CCN1 ; CCOC(=O)c1nc(Cl)nc(N2CCN(C(=O)OC(C)(C)C)C[C@@H]2C)c1[N+](=O)[O-]]

To a cooled (−60° C.) solution of ethyl 2,6-dichloro-5-nitropyrimidine-4-carboxylate (10.0 g, 0.038 mol, 1.0 eq.) in anhydrous THF (100 mL) was added a solution of tert-butyl (S)-3-methylpiperazine-1-carboxylate (7.5 g, 0.038 mol, 1.0 eq.) and DIEA (7.25 g, 0.057 mol, 1.5 eq.) in anhydrous THF (50 mL). The reaction mixture was stirred at −60° C. under Ar for 1 h. LCMS showed starting material was consumed and desired product formed. The reaction mixture was concentrated and purified by silica column chromatography eluting with Pet.ether/EtOAc (3:1, v/v) to obtain ethyl (S)-6-(4-(tert-butoxycarbonyl)-2-methylpiperazin-1-yl)-2-chloro-5-nitropyrimidine-4-carboxylate (8.6 g, 53% yield, 36-3).

LCMS (ESI, m/z): [M+1]⁺=429.9; RT=1.482 min.

### Step 2: Synthesis of Compound 36-5

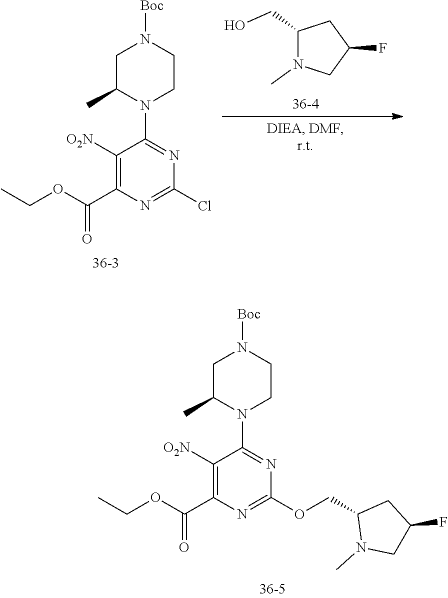

To a solution of ethyl (S)-6-(4-(tert-butoxycarbonyl)-2-methylpiperazin-1-yl)-2-chloro-5-nitropyrimidine-4-carboxylate (7.0 g, 0.016 mol, 1.0 eq.) and DIEA (4.2 g, 0.033 mol, 2.0 eq.) in anhydrous DMF (60 mL) was added ((2S,4R)-4-fluoro-1-methylpyrrolidin-2-yl) methanol (3.3 g, 0.024 mmol, 1.5 eq.). The mixture was stirred at room temperature for 16 h. LCMS showed starting material was consumed and desired product formed. The reaction mixture was diluted with H₂O (100 mL) and extracted with EtOAc (100 mL×3). The combined organic fractions were washed with brine (100 mL), dried over anhydrous Na₂SO₄ and concentrated. The residue was purified by silica column chromatography eluting with DCM/MeOH (15:1, v/v) to obtain ethyl 6-((S)-4-(tert-butoxycarbonyl)-2-methylpiperazin-1-yl)-2-(((2S,4R)-4-fluoro-1-methylpyrrolidin-2-yl)methoxy)-5-nitropyrimidine-4-carboxylate (6.7 g, 81.7% yield, 36-5).

LCMS (ESI, m/z): [M+1]⁺=527; RT=2.500 min.

### Step 3: Synthesis of Compound 36-6

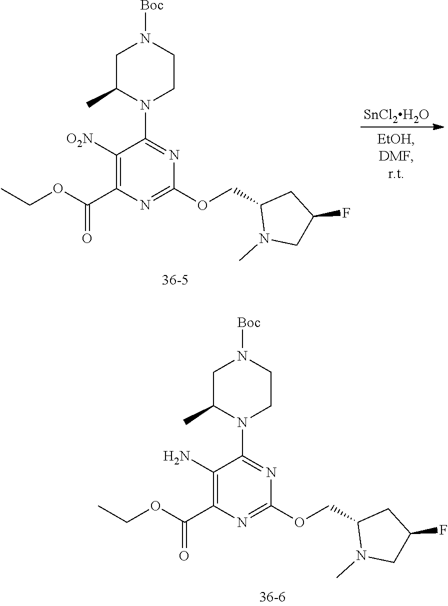 [SMILES: CCOC(=O)c1nc(OC[C@@H]2C[C@@H](F)CN2C)nc(N2CCN(C(=O)OC(C)(C)C)C[C@@H]2C)c1[N+](=O)[O-] ; "SnCl2·H2O" ; CCOC(=O)c1nc(OC[C@@H]2C[C@@H](F)CN2C)nc(N2CCN(C(=O)OC(C)(C)C)C[C@@H]2C)c1N]

To a solution of ethyl 6-((S)-4-(tert-butoxycarbonyl)-2-methylpiperazin-1-yl)-2-(((2S,4R)-4-fluoro-1-methylpyrrolidin-2-yl)methoxy)-5-nitropyrimidine-4-carboxylate (6.7 g, 0.012 mol, 1.0 eq.) in anhydrous DMF (30 mL)/EtOH (90 mL) was added SnCl₂·2H₂O (14.4 g, 0.064 mol, 5.0 eq.). The reaction mixture was stirred at room temperature under Ar for 16 h. LCMS showed that starting material was consumed and desired product formed. The reaction mixture was concentrated to remove EtOH and then diluted with EtOAc (200 mL), followed by the addition of aq. NaHCO₃ (sat. 200 mL). The resulting mixture was filtered through celite. The organic layer of the filtrate was separated and the aqueous layer was extracted with EtOAc (200 mL×2). The combined organic fractions were washed with brine (150 mL), dried over anhydrous Na₂SO₄ and concentrated. The residue was purified by silica column chromatography eluting with DCM/MeOH (15:1, v/v) to obtain ethyl 5-amino-6-((S)-4-(tert-butoxycarbonyl)-2-methylpiperazin-1-yl)-2-(((2S,4R)-4-fluoro-1-methylpyrrolidin-2-yl)methoxy)pyrimidine-4-carboxylate (3.6 g, 57% yield, 36-6).

LCMS (ESI, m/z): [M+1]⁺=497; RT=1.061 min.

### Step 4: Synthesis of Compound 36-7

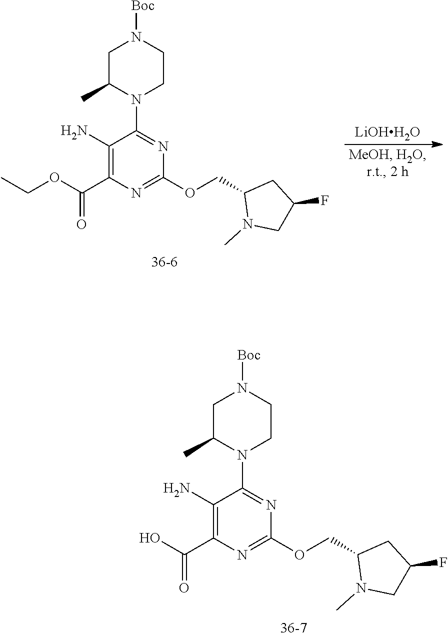 [SMILES: CCOC(=O)c1nc(OC[C@@H]2C[C@@H](F)CN2C)nc(N2CCN(C(=O)OC(C)(C)C)C[C@@H]2C)c1N ; "LiOH·H2O" ; "MeOH, H2O,r.t., 2 h" ; C[C@H]1CN(C(=O)OC(C)(C)C)CCN1c1nc(OC[C@@H]2C[C@@H](F)CN2C)nc(C(=O)O)c1N]

To a solution of ethyl 5-amino-6-((S)-4-(tert-butoxycarbonyl)-2-methylpiperazin-1-yl)-2-(((2S,4R)-4-fluoro-1-methylpyrrolidin-2-yl)methoxy)pyrimidine-4-carboxylate (3.6 g, 0.006 mol, 1.0 eq.) in MeOH (80 mL) and H₂O (8 mL) was added LiOH·H₂O (1.4 g, 0.030 mol, 5.0 eq.). The mixture was stirred at room temperature for 2 h. LCMS showed starting material was consumed and desired product formed. The reaction mixture was acidified with aq. HCl (0.5 M) to adjust pH=6, and then concentrated to dryness to concentrated to obtain 5-amino-6-((S)-4-(tert-butoxycarbonyl)-2-methylpiperazin-1-yl)-2-(((2S,4R)-4-fluoro-1-methylpyrrolidin-2-yl)methoxy)pyrimidine-4-carboxylic acid (5.4 g, crude, 36-7), which was used directly for the next step.

LCMS (ESI, m/z): [M+1]⁺=469; RT=1.020 min.

### Step 5: Synthesis of Compound 36-9

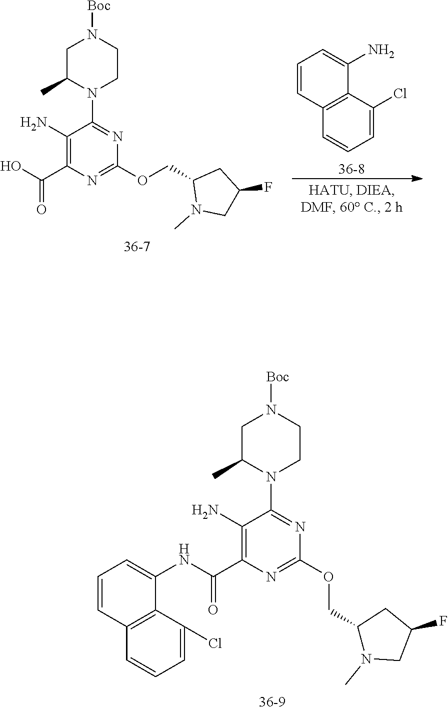

To a solution of 5-amino-6-((S)-4-(tert-butoxycarbonyl)-2-methylpiperazin-1-yl)-2-(((2S,4R)-4-fluoro-1-methylpyrrolidin-2-yl)methoxy)pyrimidine-4-carboxylic acid (0.8 g, 1.7 mmol, 1.0 eq.) and 8-methylnaphthalen-1-amine (0.74 g, 1.4 mmol, 0.8 eq.) in anhydrous DMF (10 mL) was added DIEA (0.66 g, 5.1 mmol, 3.0 eq.), followed by the addition of HATU (0.66 g, 1.7 mmol, 1.0 eq.). The reaction mixture was stirred at 60° C. under Ar for 1 h. LCMS showed starting material was consumed and desired product formed. The reaction mixture was cooled to room temperature, diluted with water (100 mL) and extracted with EtOAc (50 mL×3). The combined organic fractions were washed with brine (100 mL), dried over anhydrous Na₂SO₄ and concentrated. The residue was purified by silica column chromatography eluting with DCM/MeOH (15:1, v/v) to obtain tert-butyl (S)-4-(5-amino-6-((8-chloronaphthalen-1-yl)carbamoyl)-2-(((2S,4R)-4-fluoro-1-methylpyrrolidin-2-yl)methoxy)pyrimidin-4-yl)-3-methylpiperazine-1-carboxylate (0.66 g, 62% yield, 36-9).

LCMS (ESI, m/z): [M+1]⁺=628; RT=1.103 min.

### Step 6: Synthesis of Compound 36-10

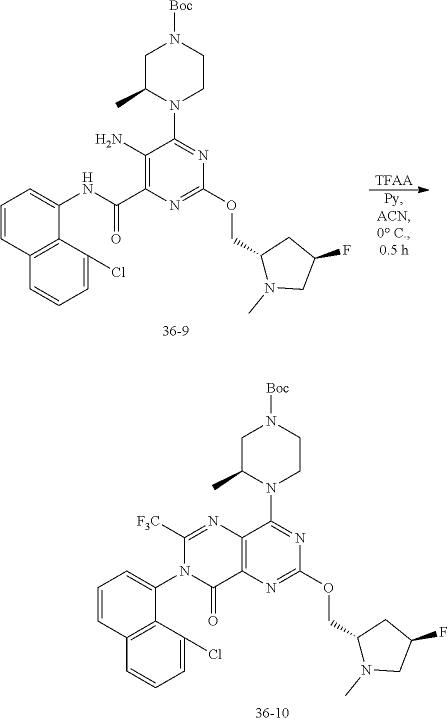 [SMILES: C[C@H]1CN(C(=O)OC(C)(C)C)CCN1c1nc(OC[C@@H]2C[C@@H](F)CN2C)nc(C(=O)Nc2cccc3cccc(Cl)c23)c1N ; "TFAA" ; "Py,ACN,0° C.,0.5 h" ; C[C@H]1CN(C(=O)OC(C)(C)C)CCN1c1nc(OC[C@@H]2C[C@@H](F)CN2C)nc2c(=O)n(-c3cccc4cccc(Cl)c34)c(C(F)(F)F)nc12]

To a cooled (0° C.) solution of tert-butyl (S)-4-(5-amino-6-((8-chloronaphthalen-1-yl)carbamoyl)-2-(((2S,4R)-4-fluoro-1-methylpyrrolidin-2-yl)methoxy)pyrimidin-4-yl)-3-methylpiperazine-1-carboxylate (0.4 g, 0.64 mmol, 1.0 eq.) in anhydrous ACN (5.0 mL) was added pyridine (0.5 g, 6.4 mmol, 10.0 eq.), followed by the addition of TFAA (0.8 g, 3.84 mmol, 6.0 eq.). The mixture was stirred at 0° C. for 0.5 h. LCMS showed starting material was consumed and desired product formed. The reaction mixture was quenched with aq. NH₄Cl (sat. 40 mL) and extracted with EtOAc (50 mL×3). The combined organic fractions were dried over anhydrous Na₂SO₄ and concentrated. The residue was purified by prep-TLC eluting with DCM/MeOH (10:1, v/v) to obtain tert-butyl (S)-4-(7-(8-chloronaphthalen-1-yl)-2-(((2S,4R)-4-fluoro-1-methylpyrrolidin-2-yl)methoxy)-8-oxo-6-(trifluoromethyl)-7,8-dihydropyrimido[5,4-d]pyrimidin-4-yl)-3-methylpiperazine-1-carboxylate (450 mg, crude, 36-10).

LCMS (ESI, m/z): [M+1]⁺=706; RT=1.154 min.

### Step 7: Synthesis of Compound 36-11

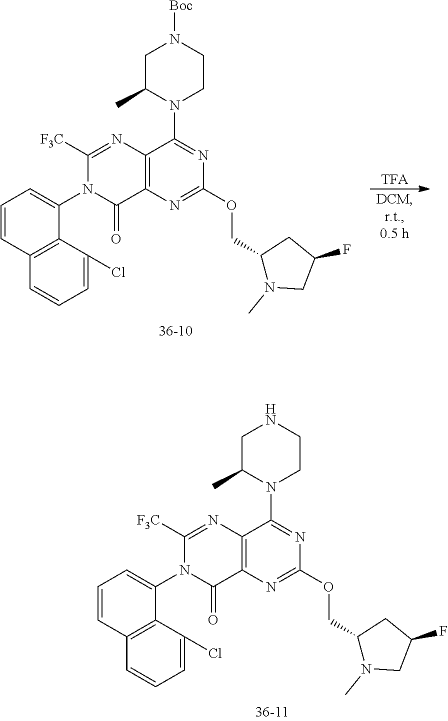 [SMILES: C[C@H]1CN(C(=O)OC(C)(C)C)CCN1c1nc(OC[C@@H]2C[C@@H](F)CN2C)nc2c(=O)n(-c3cccc4cccc(Cl)c34)c(C(F)(F)F)nc12 ; "DCM,r.t.,0.5 h" ; C[C@H]1CNCCN1c1nc(OC[C@@H]2C[C@@H](F)CN2C)nc2c(=O)n(-c3cccc4cccc(Cl)c34)c(C(F)(F)F)nc12 ; "TFA"]

To a solution of tert-butyl (S)-4-(7-(8-chloronaphthalen-1-yl)-2-(((2S,4R)-4-fluoro-1-methylpyrrolidin-2-yl)methoxy)-8-oxo-6-(trifluoromethyl)-7,8-dihydropyrimido[5,4-d]pyrimidin-4-yl)-3-methylpiperazine-1-carboxylate (450 mg, 0.64 mmol, 1.0 eq) in anhydrous DCM (6.0 mL) was added TFA (3 mL), and the mixture was stirred at room temperature for 0.5 h. LCMS showed starting material was consumed and desired product formed. The reaction mixture was treated with saturated NaHCO₃ (50 mL) extracted with EtOAc (50 mL×3). The combined organic fractions were dried over anhydrous Na₂SO₄ and concentrated. The residue was evaporated to obtain 3-(8-chloronaphthalen-1-yl)-6-(((2S,4R)-4-fluoro-1-methylpyrrolidin-2-yl)methoxy)-8-((S)-2-methylpiperazin-1-yl)-2-(trifluoromethyl)pyrimido[5,4-d]pyrimidin-4(3H)-one (390 mg, crude, 36-11).

LCMS (ESI, m/z): [M+1]⁺=606; RT=0.950 min.

### Step 8: Synthesis of Compounds 36-a and 36-b

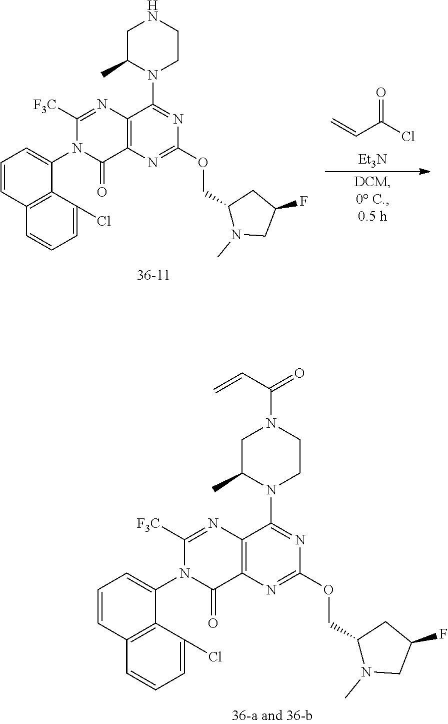

To a cooled (0° C.) solution of 3-(8-chloronaphthalen-1-yl)-6-(((2S,4R)-4-fluoro-1-methylpyrrolidin-2-yl)methoxy)-8-((S)-2-methylpiperazin-1-yl)-2-(trifluoromethyl)pyrimido[5,4-d]pyrimidin-4(3H)-one (390 mg, 0.62 mmol, 1.0 eq.) and Et₃N (187 mg, 1.24 mmol, 3.0 eq.) in DCM (5 mL) was added dropwise a solution of acryloyl chloride (88.0 mg, 0.93 mmol, 1.5 eq.) in DCM (1 mL). After addition, the mixture was stirred at 0° C. for 30 min. LCMS showed starting material was consumed and desired product formed. Water (20 mL) was added and the organic layer was separated. The aqueous layer was extracted with DCM (50 mL×2). The combined organic fractions were dried over anhydrous Na₂SO₄ and concentrated. The residue was purified by TLC with DCM/MeOH (15:1, v/v) and then SFC to obtain 8-((S)-4-acryloyl-2-methylpiperazin-1-yl)-3-(8-chloronaphthalen-1-yl)-6-(((2S,4R)-4-fluoro-1-methylpyrrolidin-2-yl)methoxy)-2-(trifluoromethyl)pyrimido[5,4-d]pyrimidin-4(3H)-one (34.63 mg, 19% yield, 36-a) and 8-((S)-4-acryloyl-2-methylpiperazin-1-yl)-3-(8-chloronaphthalen-1-yl)-6-(((2S,4R)-4-fluoro-1-methylpyrrolidin-2-yl)methoxy)-2-(trifluoromethyl)pyrimido[5,4-d]pyrimidin-4(3H)-one (48.56 mg, 19% yield, 36-b).

36-a:

LCMS (ESI, m/z): [M+1]⁺=660; RT=1.228 min;

¹H NMR (400 MHz, CDCl3) δ 8.08 (d, J=8.1 Hz, 1H), 7.90 (d, J=8.1 Hz, 1H), 7.62 (t, J=7.8 Hz, 1H), 7.57 (d, J=7.4 Hz, 1H), 7.44 (t, J=6.4 Hz, 2H), 6.61 (dd, J=27.4, 16.7 Hz, 1H), 6.40 (d, J=16.9 Hz, 1H), 5.78 (d, J=10.7 Hz, 1H), 5.56 (s, 1H), 5.17 (d, J=56.5 Hz, 1H), 4.77-4.25 (m, 3H), 3.94 (dd, J=60.2, 9.3 Hz, 1H), 3.73-2.91 (m, 5H), 2.60 (d, J=44.4 Hz, 4H), 2.28 (d, J=23.6 Hz, 1H), 2.04 (s, 1H), 1.38 (dd, J=21.2, 12.4 Hz, 4H).

¹⁹F NMR (377 MHz, CDCl3) δ −64.81 (d, J=12.6 Hz).

36-b:

LCMS (ESI, m/z): [M+1]⁺=660; RT=1.223 min;

¹H NMR (400 MHz, CDCl3) δ 8.08 (d, J=7.9 Hz, 1H), 7.90 (d, J=7.9 Hz, 1H), 7.62 (t, J=7.8 Hz, 1H), 7.57 (d, J=6.8 Hz, 1H), 7.45 (dd, J=10.1, 5.5 Hz, 2H), 6.69-6.53 (m, 1H), 6.40 (d, J=16.6 Hz, 1H), 5.78 (d, J=10.2 Hz, 1H), 5.55 (s, 1H), 5.15 (d, J=55.5 Hz, 1H), 4.65-4.33 (m, 3H), 4.09-3.80 (m, 1H), 3.70-2.84 (m, 5H), 2.59 (ddd, J=32.4, 11.8, 2.7 Hz, 1H), 2.50 (s, 3H), 2.35-2.21 (m, 1H), 1.99 (dddd, J=25.6, 20.9, 13.5, 8.5 Hz, 1H), 1.39 (dd, J=28.3, 12.4 Hz, 4H).

¹⁹F NMR (377 MHz, CDCl3) δ −64.86 (s).

### Example 36

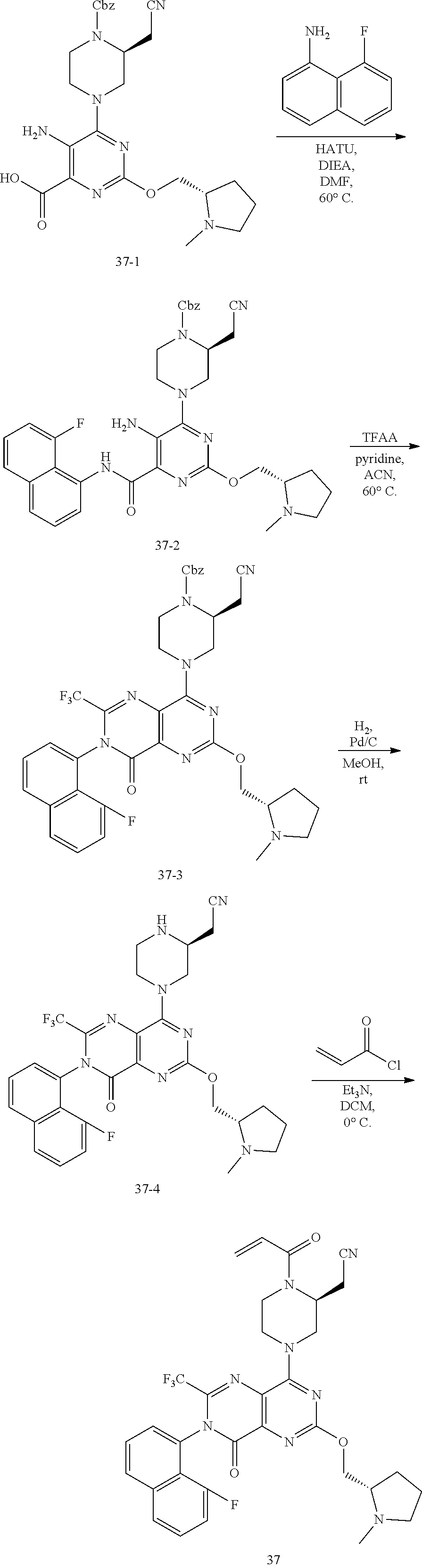

### Step 1: Synthesis of Compound 37-2

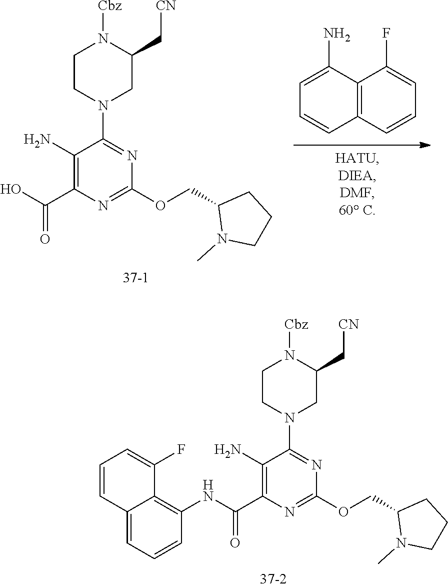 [SMILES: CN1CCC[C@H]1COc1nc(C(=O)O)c(N)c(N2CCN(C(=O)OCc3ccccc3)[C@@H](CC#N)C2)n1 ; Nc1cccc2cccc(F)c12 ; CN1CCC[C@H]1COc1nc(C(=O)Nc2cccc3cccc(F)c23)c(N)c(N2CCN(C(=O)OCc3ccccc3)[C@@H](CC#N)C2)n1]

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (800 mg, 1.57 mmol, 1.0 eq.) and 8-fluoronaphthalen-1-amine (152 mg, 0.94 mmol, 0.6 eq.) in anhydrous DMF (8.0 mL) was added DIEA (1.02 g, 7.85 mmol, 5.0 eq.), followed by the addition of HATU (597 mg, 1.57 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (50 mL×3). The combined organic fractions were washed with brine (20 mL), dried over anhydrous Na₂SO₄ and concentrated. The residue was purified by silica column chromatography eluting with DCM/MeOH (15:1, v/v) to obtain benzyl (S)-4-(5-amino-6-((8-fluoronaphthalen-1-yl)carbamoyl)-2-(((S)-1-methylpyrrolidin-2-yl)methoxy) pyrimidin-4-yl)-2-(cyanomethyl)piperazine-1-carboxylate (284 mg, 28% yield, 37-2).

LCMS (ESI, m/z): [M+1]⁺=653; RT=1.176 min.

### Step 2: Synthesis of Compound 37-3

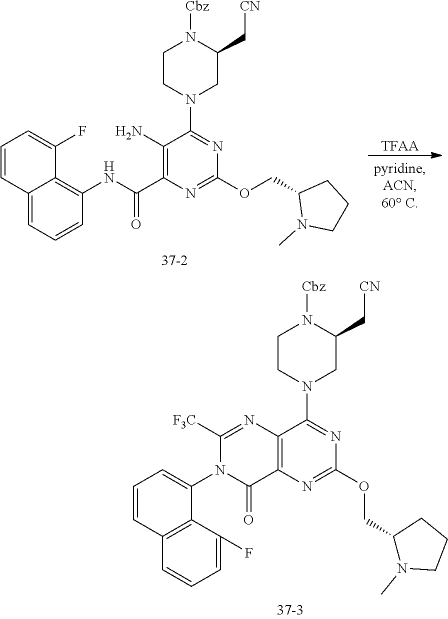 [SMILES: CN1CCC[C@H]1COc1nc(C(=O)Nc2cccc3cccc(F)c23)c(N)c(N2CCN(C(=O)OCc3ccccc3)[C@@H](CC#N)C2)n1 ; "TFAA" ; CN1CCC[C@H]1COc1nc(N2CCN(C(=O)OCc3ccccc3)[C@@H](CC#N)C2)c2nc(C(F)(F)F)n(-c3cccc4cccc(F)c34)c(=O)c2n1]

To a mixture of benzyl (S)-4-(5-amino-6-((8-fluoronaphthalen-1-yl)carbamoyl)-2-(((S)-1-methylpyrrolidin-2-yl)methoxy) pyrimidin-4-yl)-2-(cyanomethyl)piperazine-1-carboxylate (260 mg, 0.40 mmol, 1.0 eq.) in ACN (2.0 mL), was added pyridine (316 mg, 4.0 mmol, 10.0 eq.) and TFAA (502 mg, 2.4 mmol, 6.0 eq). The mixture was stirred at 0° C. for 25 min and 60° C. for 1 h. LCMS showed starting material was consumed and desired product formed. The reaction mixture was concentrated and purified by prep-TLC eluting with DCM/MeOH (10:1, v/v) to obtain benzyl (S)-2-(cyanomethyl)-4-(7-(8-fluoronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazine-1-carboxylate (76 mg, 26% yield, 37-3).

LCMS (ESI, m/z): [M+1]⁺=731; RT=1.260 min.

### Step 3: Synthesis of Compound 37-4

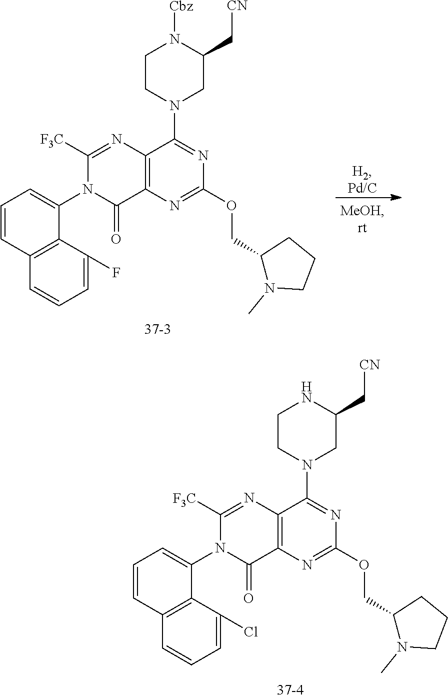 [SMILES: CN1CCC[C@H]1COc1nc(N2CCN(C(=O)OCc3ccccc3)[C@@H](CC#N)C2)c2nc(C(F)(F)F)n(-c3cccc4cccc(F)c34)c(=O)c2n1 ; "H2,Pd/C" ; CN1CCC[C@H]1COc1nc(N2CCN[C@@H](CC#N)C2)c2nc(C(F)(F)F)n(-c3cccc4cccc(Cl)c34)c(=O)c2n1]

To a solution of benzyl (S)-2-(cyanomethyl)-4-(7-(8-fluoronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazine-1-carboxylate (76 mg, 0.1 mmol) in MeOH (5.0 mL) was Pd/C (50 mg), and the mixture was stirred at room temperature for 1 h under H₂. LCMS showed starting material was consumed and desired product formed. The resulting mixture was filtered through celite. The organic layer of the filtrate was concentrated obtain 2-((S)-4-(7-(8-fluoronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl) acetonitrile (60 mg, 97% yield, 37-4), which was used directly for the next step.

### Step 4: Synthesis of Compound 37

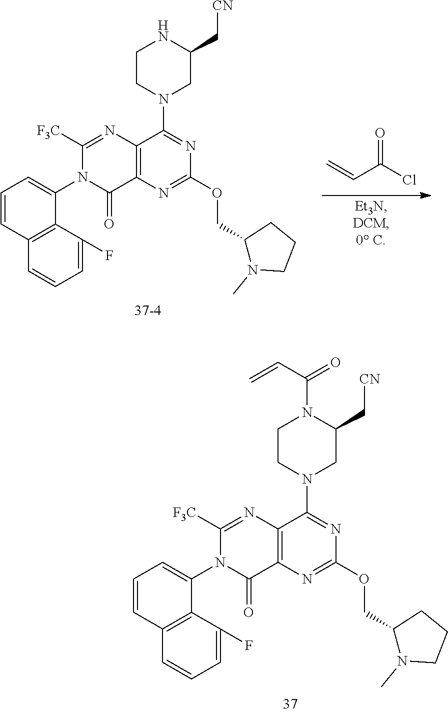

To a cooled (0° C.) solution of 2-((S)-4-(7-(8-fluoronaphthalen-1-yl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl) acetonitrile (60 mg, 0.1 mmol, 1.0 eq.) and Et₃N (51 mg, 0.5 mmol, 5.0 eq.) in DCM (2.5 mL) was added dropwise a solution of acryloyl chloride (11 mg, 0.12 mmol, 1.2 eq.) in DCM (0.5 mL). After addition, the mixture was stirred at 0° C. for 30 min. LCMS showed starting material was consumed and desired product formed. Water (15 mL) was added and the organic layer was separated. The aqueous layer was extracted with DCM (8 mL×3). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(8-fluoronaphthalen-1-yl)-2-(((S)-1-methylpyrrolidin-2-yl) methoxy)-8-oxo-6-(trifluoromethyl)-7,8-dihydropyrimido[5,4-d] pyrimidin-4-yl) piperazin-2-yl) acetonitrile (HCOOH salt, 5.19 mg, 7.9% yield, 37) (C₃₂H₃₀F₄N₈O₃·HCOOH).

LCMS (ESI, m/z): [M+1]⁺=651; RT=1.627 min;

¹H NMR (400 MHz, CDCl₃) δ 8.06 (d, J=8 Hz, 1H), 7.77 (d, J=8 Hz, 1H), 7.64 (t, J=8 Hz, 1H), 7.52-7.43 (m, 2H), 7.17-7.11 (m, 1H), 6.66-6.59 (m, 1H), 6.42 (d, J=16 Hz, 1H), 5.85 (d, J=12 Hz, 1H), 5.50-5.10 (m, 2H), 4.68-4.49 (m, 2H), 4.05-3.76 (m, 2H), 3.54-3.32 (m, 2H), 2.94-2.83 (m, 3H), 2.64 (s, 3H), 2.64-2.29 (m, 1H), 2.12-1.87 (m, 6H).

¹⁹F NMR (400 MHz, CDCl₃) δ −64.97, −121.59.

### Example 37

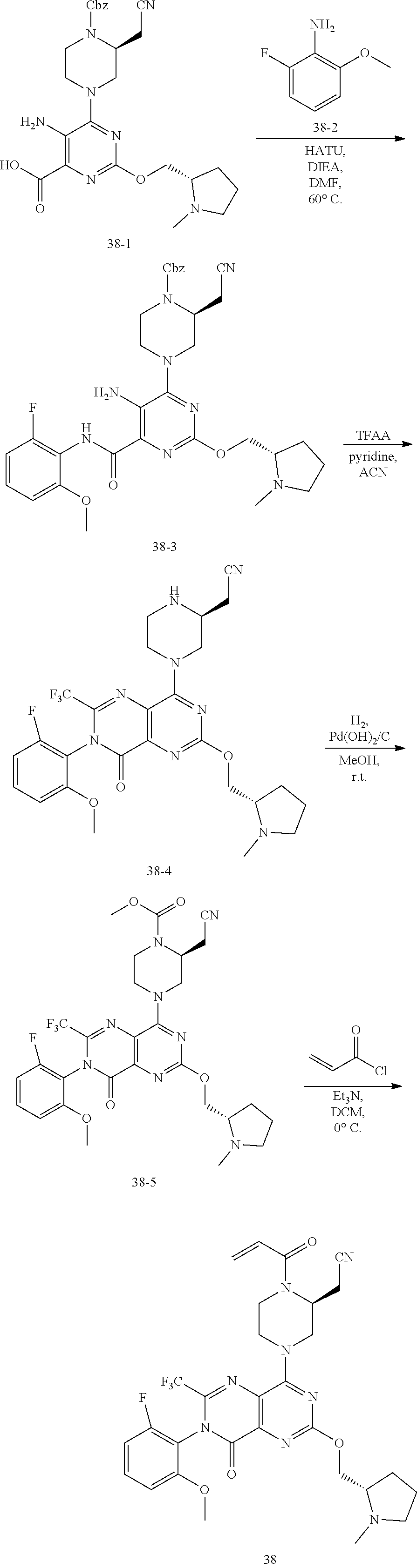

### Step 1: Synthesis of Compound 38-3

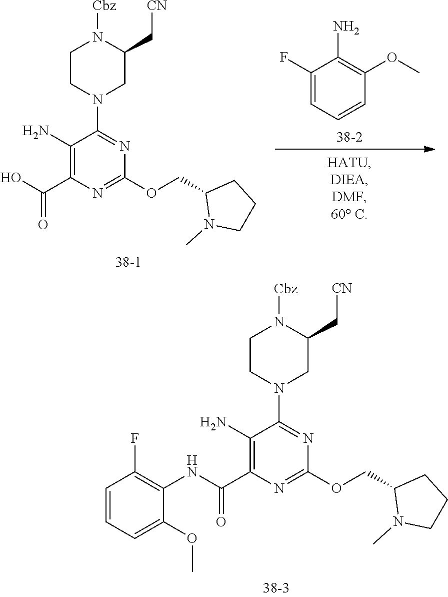

To a solution of 5-amino-6-((S)-4-((benzyloxy)carbonyl)-3-(cyanomethyl)piperazin-1-yl)-2-(((S)-1-methylpyrrolidin-2-yl)methoxy)pyrimidine-4-carboxylic acid (1.20 g, 2.35 mmol, 1.0 eq.), 2-fluoro-6-methoxyaniline (266 mg, 1.88 mmol, 0.8 eq.) and DIEA (1.2 mL, 7.05 mmol, 3.0 eq.) in anhydrous DMF (10 mL) was added HATU (894 mg, 2.35 mmol, 1.0 eq.). The mixture was stirred at 60° C. for 1 h. LCMS showed starting material was consumed and desired product formed. The reaction mixture was cooled to r.t., diluted with water (80 mL) and extracted with EtOAc (40 mL×3). The combined organic fractions were washed with brine (80 mL), dried over anhydrous Na₂SO₄ and concentrated. The residue was purified by silica column chromatography, eluting with DCM/MeOH (v/v, 10:1) to obtain benzyl (S)-4-(5-amino-6-((2-fluoro-6-methoxyphenyl)carbamoyl)-2-(((S)-1-methylpyrrolidin-2-yl)methoxy)pyrimidin-4-yl)-2-(cyanomethyl)piperazine-1-carboxylate (468 mg, 31%, 38-3).

LCMS (ESI, m/z): [M+1]⁺=633; RT=1.086 min.

### Step 2: Synthesis of Compound 38-4

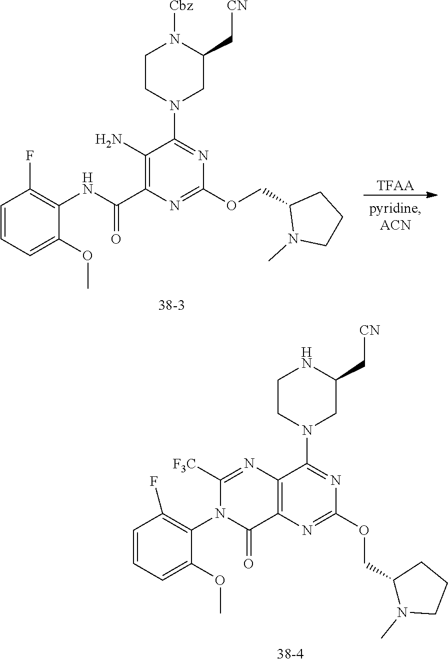 [SMILES: COc1cccc(F)c1NC(=O)c1nc(OC[C@@H]2CCCN2C)nc(N2CCN(C(=O)OCc3ccccc3)[C@@H](CC#N)C2)c1N ; "TFAA" ; COc1cccc(F)c1-n1c(C(F)(F)F)nc2c(N3CCN[C@@H](CC#N)C3)nc(OC[C@@H]3CCCN3C)nc2c1=O]

To a cooled (0° C.) solution of benzyl (S)-4-(5-amino-6-((2-fluoro-6-methoxyphenyl)carbamoyl)-2-(((S)-1-methylpyrrolidin-2-yl)methoxy)pyrimidin-4-yl)-2-(cyanomethyl)piperazine-1-carboxylate (468 mg, 0.740 mmol, 1.0 eq.) and pyridine (1.17 g, 14.8 mmol, 20.0 eq.) in anhydrous ACN (10 mL) was added TFAA (1.86 g, 8.88 mmol, 12.0 eq.) dropwise. After addition, the mixture was stirred at 0° C. for 30 min, and then 60° C. for 1 h. LCMS showed starting material was consumed and desired product formed. The reaction mixture was cooled to r.t. and concentrated to dryness. The residue was purified by prep-TLC, eluting with DCM/MeOH (v/v, 10:1) to obtain benzyl (S)-2-(cyanomethyl)-4-(7-(2-fluoro-6-methoxyphenyl)-2-(((S)-1-methyl pyrrolidin-2-yl)methoxy)-8-oxo-6-(trifluoromethyl)-7,8-dihydropyrimido[5,4-d]pyrimidin-4-yl)piperazine-1-carboxylate (257 mg, 49%, 38-4).

LCMS (ESI, m/z): [M+1]⁺=711; RT=1.255 min.

### Step 3: Synthesis of Compound 38-5

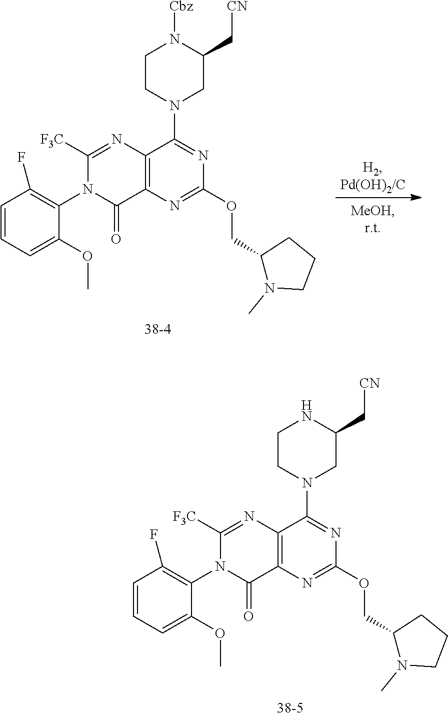 [SMILES: COc1cccc(F)c1-n1c(C(F)(F)F)nc2c(N3CCN(C(=O)OCc4ccccc4)[C@@H](CC#N)C3)nc(OC[C@@H]3CCCN3C)nc2c1=O ; "H2,Pd(OH)2/C" ; COc1cccc(F)c1-n1c(C(F)(F)F)nc2c(N3CCN[C@@H](CC#N)C3)nc(OC[C@@H]3CCCN3C)nc2c1=O]

To a solution of benzyl (S)-2-(cyanomethyl)-4-(7-(2-fluoro-6-methoxyphenyl)-2-(((S)-1-methyl pyrrolidin-2-yl)methoxy)-8-oxo-6-(trifluoromethyl)-7,8-dihydropyrimido[5,4-d]pyrimidin-4-yl)piperazine-1-carboxylate (257 mg, 0.362 mmol, 1.0 eq.) in MeOH (5 mL) was added Pd(OH)₂/C (20% w/w, 25 mg, 36.2 μmol, 0.1 eq.), and the mixture was stirred at r.t. under H₂ (balloon) for 1 h. LCMS showed most of starting material was consumed and desired product formed. The reaction mixture was filtered through celite. The filtrate was concentrated to dryness to obtain 2-((S)-4-(7-(2-fluoro-6-methoxyphenyl)-2-(((S)-1-methylpyrrolidin-2-yl)methoxy)-8-oxo-6-(trifluoromethyl)-7,8-dihydropyrimido[5,4-d]pyrimidin-4-yl) piperazin-2-yl)acetonitrile (234 mg, crude, 38-5), which was used directly for the next step.

LCMS (ESI, m/z): [M+1]⁺=577; RT=0.701 min.

### Step 4: Synthesis of Compound 38

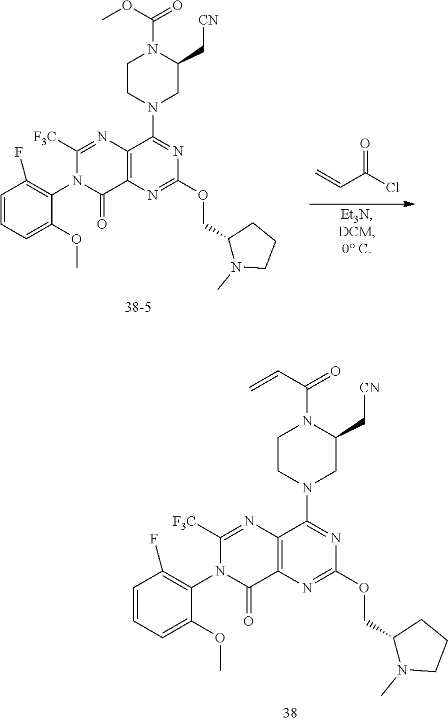

To a cooled (0° C.) solution of 2-((S)-4-(7-(2-fluoro-6-methoxyphenyl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (234 mg, 0.406 mmol, 1.0 eq.) and Et₃N (82 mg, 0.812 mmol, 2.0 eq.) in anhydrous DCM (3 mL) was added dropwise a solution of acryloyl chloride (18 mg, 0.203 mmol, 0.5 eq.) in anhydrous DCM (0.5 mL). After addition, the mixture was stirred at 0° C. for 30 min. LCMS showed most of starting material was consumed and desired product formed. Water (20 mL) was added and the organic layer was separated. The aqueous layer was extracted with DCM (10 mL×3). The combined organic fractions were washed with brine (20 mL), dried over anhydrous Na₂SO₄ and concentrated. The residue was purified by prep-HPLC (ACN-H₂O+0.1% HCOOH) to obtain 2-((S)-1-acryloyl-4-(7-(2-fluoro-6-methoxyphenyl)-2-(((S)-1-methylpyrrolidin-2-yl)methoxy)-8-oxo-6-(trifluoromethyl)-7,8-dihydropyrimido[5,4-d]pyrimidin-4-yl)piperazin-2-yl)acetonitrile (HCOOH salt, 10.08 mg, 3.6%, 38) (C₂₉H₃₀F₄N₈O₄·0.4HCOOH).

LCMS (ESI, m/z): [M+1]⁺=631; RT=1.589 min.

¹H NMR (400 MHz, DMSO-d₆) δ 8.27 (s, 1H), 7.65 (dd, J=15.3, 8.5 Hz, 1H), 7.21-7.07 (m, 2H), 6.97-6.82 (m, 1H), 6.20 (d, J=16.4 Hz, 1H), 5.78 (d, J=9.8 Hz, 1H), 5.39-4.83 (m, 3H), 4.55-4.29 (m, 2H), 4.26-4.07 (m, 2H), 3.80 (d, J=2.1 Hz, 3H), 3.64-3.52 (m, 2H), 3.21-3.14 (m, 1H), 3.00-2.88 (m, 2H), 2.61-2.54 (m, 1H), 2.36 (s, 3H), 2.19 (q, J=8.7 Hz, 1H), 2.00-1.89 (m, 1H), 1.73-1.56 (m, 3H).

¹⁹F NMR (376 MHz, DMSO-d₆) δ −67.03, −120.76.

### Example 38

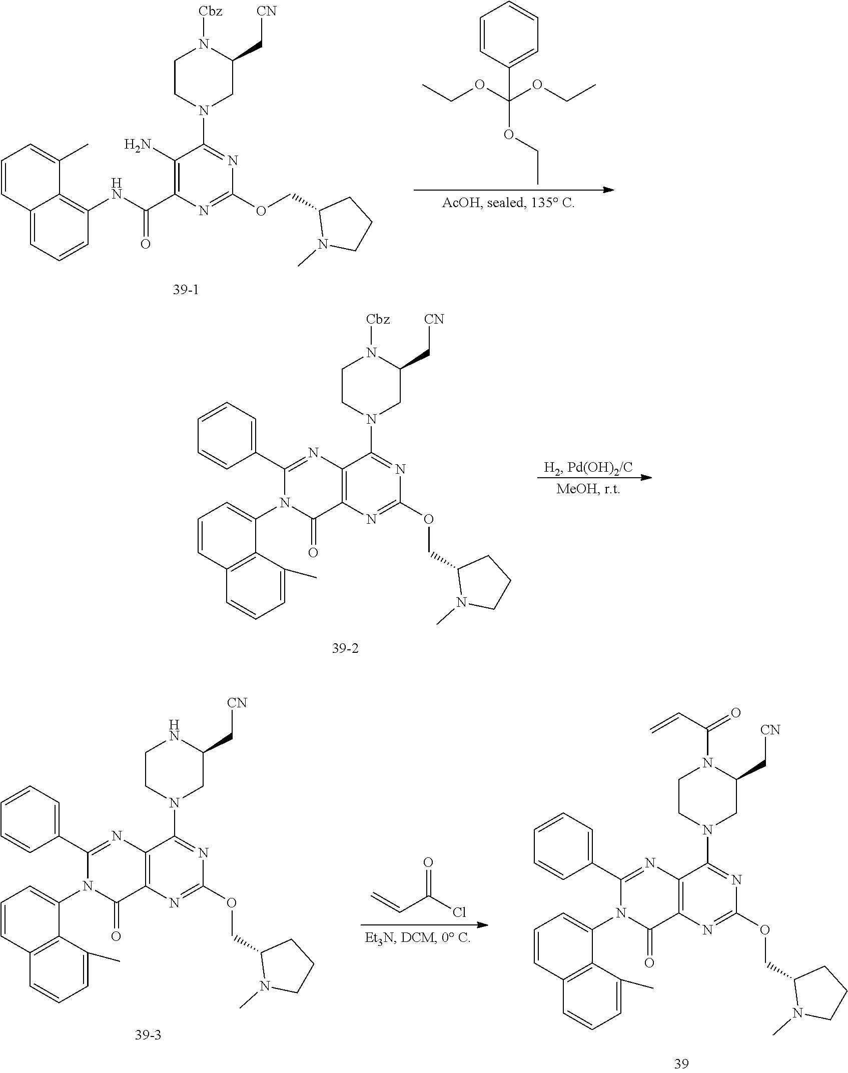

### Step 1: Synthesis of Compound 39-2

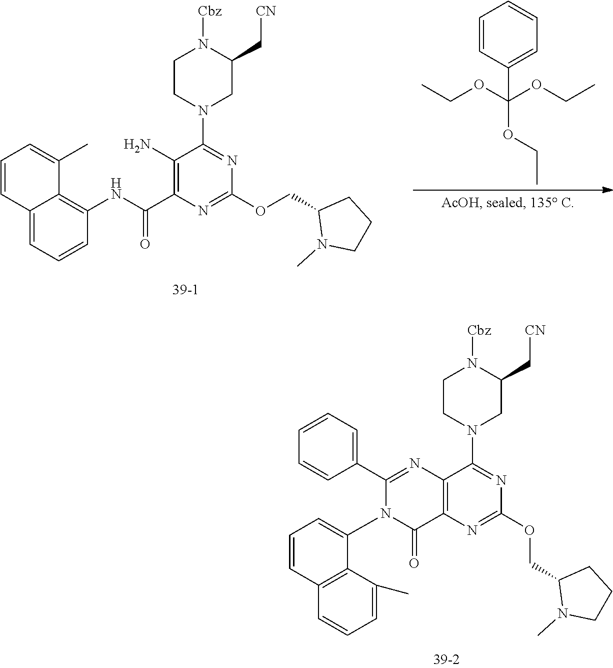 [SMILES: Cc1cccc2cccc(NC(=O)c3nc(OC[C@@H]4CCCN4C)nc(N4CCN(C(=O)OCc5ccccc5)[C@@H](CC#N)C4)c3N)c12 ; CCOC(OCC)(OCC)c1ccccc1 ; "AcOH, sealed, 135° C." ; Cc1cccc2cccc(-n3c(-c4ccccc4)nc4c(N5CCN(C(=O)OCc6ccccc6)[C@@H](CC#N)C5)nc(OC[C@@H]5CCCN5C)nc4c3=O)c12]

To a mixture of benzyl (S′)-4-(5-amino-6-((8-methylnaphthalen-1-yl)carbamoyl)-2-(((S)-1-methylpyrrolidin-2-yl)methoxy)pyrimidin-4-yl)-2-(cyanome1,1,1-triethoxypropanehyl)piperazine-1-carboxylate (251 mg, 0.387 mmol, 1.0 eq.) and AcOH (2.5 mL) was added (triethoxymethyl)benzene (1.30 g, 5.80 mmol, 15.0 eq.). The mixture was stirred at 135° C. in a sealed tube for 3 min LCMS showed starting material was consumed and desired product formed. The reaction mixture was quenched with aq. NaHCO₃ (sat. 150 mL) to adjust pH=7-8, which was extracted with DCM (30 mL×2). The combined organic fractions were dried over anhydrous Na₂SO₄ and concentrated. The residue was purified by prep-TLC eluting with DCM/MeOH (10:1, v/v) to obtain benzyl (S)-2-(cyanomethyl)-4-(7-(8-methylnaphthalen-1-yl)-2-(((S)-1-methylpyrrolidin-2-yl)methoxy)-8-oxo-6-phenyl-7,8-dihydropyrimido[5,4-d]pyrimidin-4-yl)piperazine-1-carboxylate (120 mg, 42%, 39-2).

LCMS (ESI, m/z): [M+1]⁺=735; RT=1.302 min.

### Step 2: Synthesis of Compound 39-3

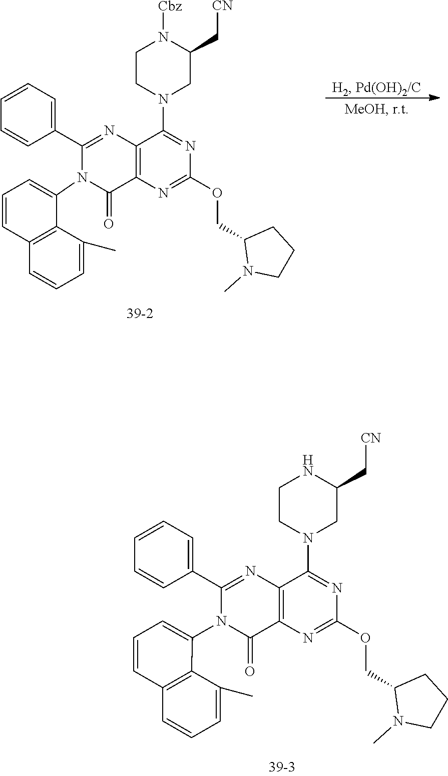 [SMILES: Cc1cccc2cccc(-n3c(-c4ccccc4)nc4c(N5CCN(C(=O)OCc6ccccc6)[C@@H](CC#N)C5)nc(OC[C@@H]5CCCN5C)nc4c3=O)c12 ; "H2, Pd(OH)2/C" ; "MeOH, r.t." ; Cc1cccc2cccc(-n3c(-c4ccccc4)nc4c(N5CCN[C@@H](CC#N)C5)nc(OC[C@@H]5CCCN5C)nc4c3=O)c12]

To a solution of benzyl (S)-2-(cyanomethyl)-4-(7-(8-methylnaphthalen-1-yl)-2-(((S)-1-methylpyrrolidin-2-yl)methoxy)-8-oxo-6-phenyl-7,8-dihydropyrimido[5,4-d]pyrimidin-4-yl)piperazine-1-carboxylate (120 mg, 0.163 mmol, 1.0 eq.) in MeOH (5.0 mL) was added Pd(OH)₂ (20% w/w, 11.5 mg, 0.0163 mmol, 0.1 eq.). The reaction mixture was stirred at room temperature under H₂ (balloon) for 1 h. LCMS showed starting material was consumed and desired product formed. The mixture was filtered through celite and the filtrate was concentrated to dryness to obtain 2-((S)-4-(7-(8-methylnaphthalen-1-yl)-2-(((S)-1-methylpyrrolidin-2-yl)methoxy)-8-oxo-6-phenyl-7,8-dihydropyrimido[5,4-d]pyrimidin-4-yl)piperazin-2-yl)acetonitrile (96 mg, 98%, 39-3), which was used directly for the next step.

LCMS (ESI, m/z): [M+1]⁺=601; RT=0.828 min.

### Step 3: Synthesis of Compound 39

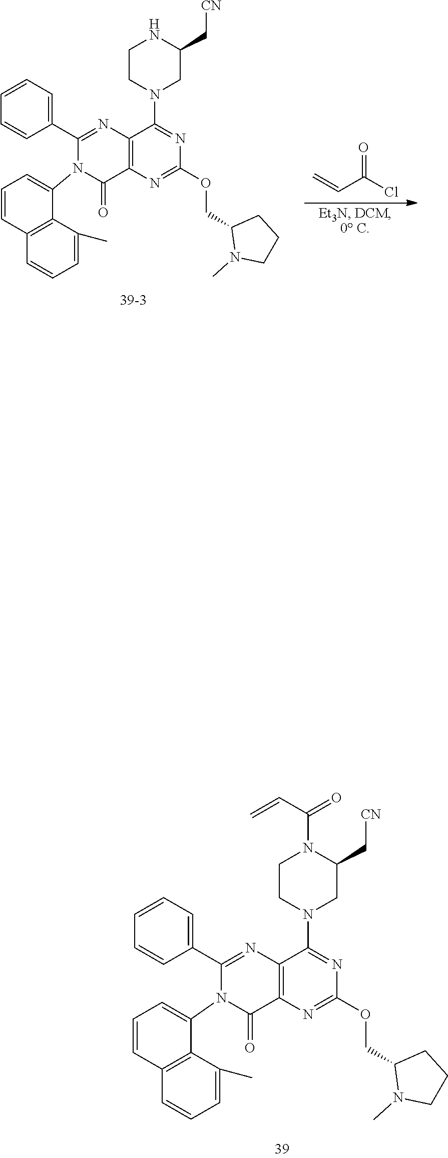

To a cooled (0° C.) solution of 2-((S)-4-(7-(8-methylnaphthalen-1-yl)-2-(((S)-1-methylpyrrolidin-2-yl)methoxy)-8-oxo-6-phenyl-7,8-dihydropyrimido[5,4-d]pyrimidin-4-yl)piperazin-2-yl)acetonitrile (96 mg, 0.160 mmol, 1.0 eq.) and Et₃N (81 mg, 0.799 mmol, 5.0 eq.) in anhydrous DCM (3.0 mL) was added dropwise a solution of acryloyl chloride (17.4 mg, 0.192 mmol, 1.2 eq.) in anhydrous DCM (0.5 mL). After addition, the mixture was stirred at 0° C. for 30 min. LCMS showed most of starting material was consumed and desired product formed. Water (10 mL) was added and the organic layer was separated. The aqueous layer was extracted with DCM (5 mL×2). The combined organic fractions were dried over anhydrous Na₂SO₄ and concentrated. The residue was purified by prep-HPLC (ACN-H₂O+0.1% HCOOH) to obtain 2-((S)-1-acryloyl-4-(7-(8-methylnaphthalen-1-yl)-2-(((S)-1-methylpyrrolidin-2-yl)methoxy)-8-oxo-6-phenyl-7,8-dihydropyrimido[5,4-d]pyrimidin-4-yl)piperazin-2-yl)acetonitrile (HCOOH salt, 16.46 mg, 15%, 39) (C₃₄H₃₈N₈O₃·0.9HCOOH).

LCMS (ESI, m/z): [M+1]⁺=655; RT=1.670 min.

¹H NMR (400 MHz, DMSO-d₆) δ 8.22 (s, 1H), 7.93 (d, J=8.4 Hz, 1H), 7.84 (t, J=7.6 Hz, 1H), 7.53-7.34 (m, 4H), 7.25-7.13 (m, 3H), 7.09 (d, J=6.3 Hz, 2H), 6.87 (dd, J=26.0, 14.6 Hz, 1H), 6.19 (d, J=16.5 Hz, 1H), 5.76 (d, J=10.4 Hz, 1H), 5.40-4.78 (m, 3H), 4.49-4.33 (m, 2H), 4.27-4.07 (m, 3H), 3.65-3.47 (m, 2H), 3.17 (s, 1H), 3.03-2.96 (m, 1H), 2.88-2.73 (m, 1H), 2.70-2.61 (m, 1H), 2.41 (s, 3H), 2.37 (s, 2H), 2.24 (dd, J=16.4, 8.3 Hz, 1H), 2.02-1.93 (m, 1H), 1.77-1.58 (m, 3H).

### Example 39

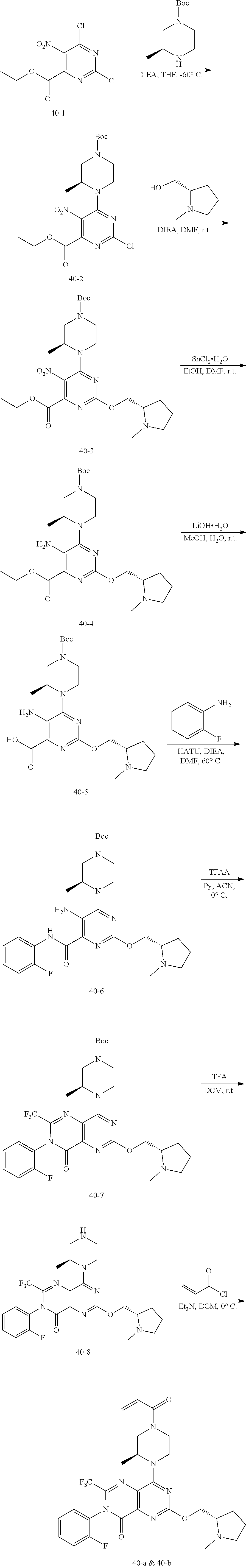

### Step 1: Synthesis of Compound 40-2

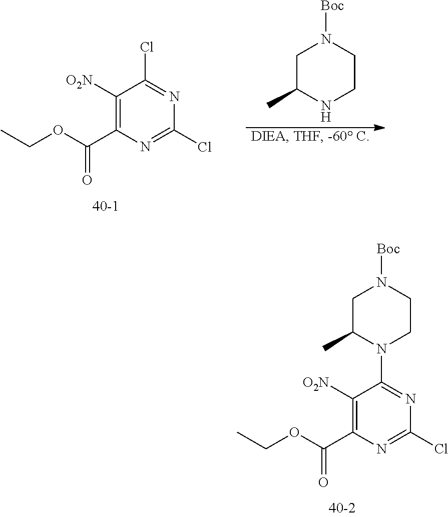

To a cooled (−60° C.) solution of ethyl 2,6-dichloro-5-nitropyrimidine-4-carboxylate (5.0 g, 0.019 mol, 1.0 eq.) in anhydrous THF (50 mL) was added dropwise a solution of tert-butyl (S)-3-methylpiperazine-1-carboxylate (3.75 g, 0.019 mol, 1.0 eq.) and DIEA (4.6 mL, 0.028 mol, 1.5 eq.) in anhydrous THF (30 mL). The mixture was stirred at −60° C. under Ar for 1 h. LCMS showed starting material was consumed and desired product formed. The reaction mixture was concentrated and the residue was purified by silica column chromatography eluting with Pet. ether/EtOAc (3:1, v/v) to obtain ethyl (S)-6-(4-(tert-butoxycarbonyl)-2-methylpiperazin-1-yl)-2-chloro-5-nitropyrimidine-4-carboxylate (8.2 g, crude, 40-2).

LCMS (ESI, m/z): [M+1]⁺=430; RT=2.141 min.

### Step 2: Synthesis of Compound 40-3

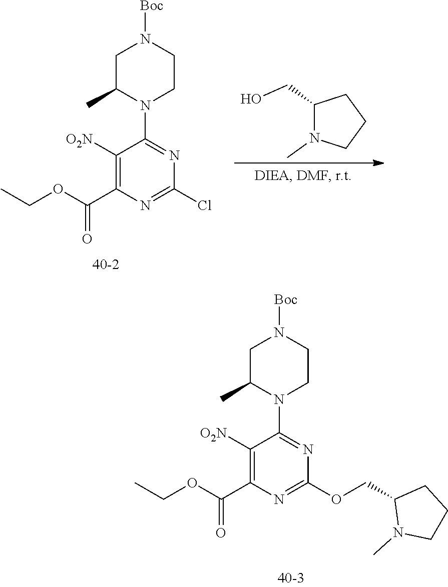

To a solution of ethyl (S)-6-(4-(tert-butoxycarbonyl)-2-methylpiperazin-1-yl)-2-chloro-5-nitropyrimidine-4-carboxylate (8.2 g, 0.019 mol, 1.0 eq.) and DIEA (6.3 ml, 0.038 mol, 2.0 eq.) in anhydrous DMF (60.0 mL) was added (S)-(1-methylpyrrolidin-2-yl) methanol (3.3 g, 0.029 mol, 1.5 eq.). The mixture was stirred at room temperature for 16 h. LCMS showed starting material was consumed and desired product formed. The reaction mixture was diluted with H₂O (100 mL) and extracted with EtOAc (80 mL×3). The combined organic fractions were washed with brine (100 mL), dried over anhydrous Na₂SO₄ and concentrated. The residue was purified by silica column chromatography eluting with DCM/MeOH (15:1, v/v) to obtain ethyl 6-((S)-4-(tert-butoxycarbonyl)-2-methylpiperazin-1-yl)-2-(((S)-1-methylpyrrolidin-2-yl) methoxy)-5-nitropyrimidine-4-carboxylate (8.8 g, 91% yield, 40-3).

LCMS (ESI, m/z): [M+1]⁺=509; RT=1.099 min.

### Step 3: Synthesis of Compound 40-4

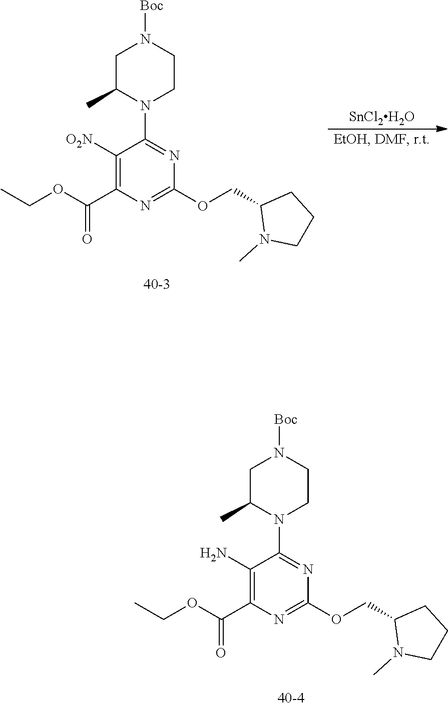 [SMILES: CCOC(=O)c1nc(OC[C@@H]2CCCN2C)nc(N2CCN(C(=O)OC(C)(C)C)C[C@@H]2C)c1[N+](=O)[O-] ; "SnCl2·H2O" ; "EtOH, DMF, r.t." ; CCOC(=O)c1nc(OC[C@@H]2CCCN2C)nc(N2CCN(C(=O)OC(C)(C)C)C[C@@H]2C)c1N]

To a solution of ethyl 6-((S)-4-(tert-butoxycarbonyl)-2-methylpiperazin-1-yl)-2-(((S)-1-methylpyrrolidin-2-yl) methoxy)-5-nitropyrimidine-4-carboxylate (8.8 g, 0.017 mol, 1.0 eq.) in anhydrous DMF (20 mL)/EtOH (60 mL) was added SnCl₂·2H₂O (19.6 g, 0.087 mol, 5.0 eq.). The mixture was stirred at room temperature under Ar for 16 h. LCMS showed that starting material was consumed and desired product formed. The reaction mixture was concentrated to remove EtOH and then diluted with EtOAc (120 mL), followed by the addition of aq. NaHCO₃ (sat. 180 mL). The resulting mixture was filtered through celite. The organic layer of the filtrate was separated and the aqueous layer was extracted with EtOAc (100 mL×2). The combined organic fractions were washed with brine (160 mL), dried over anhydrous Na₂SO₄ and concentrated. The residue was purified by silica column chromatography eluting with DCM/MeOH (15:1, v/v) to obtain ethyl 5-amino-6-((S)-4-(tert-butoxycarbonyl)-2-methylpiperazin-1-yl)-2-(((S)-1-methylpyrrolidin-2-yl) methoxy) pyrimidine-4-carboxylate (3.3 g, 40% yield, 40-4).

LCMS (ESI, m/z): [M+1]⁺=479; RT=0.867 min.

### Step 4: Synthesis of Compound 40-5

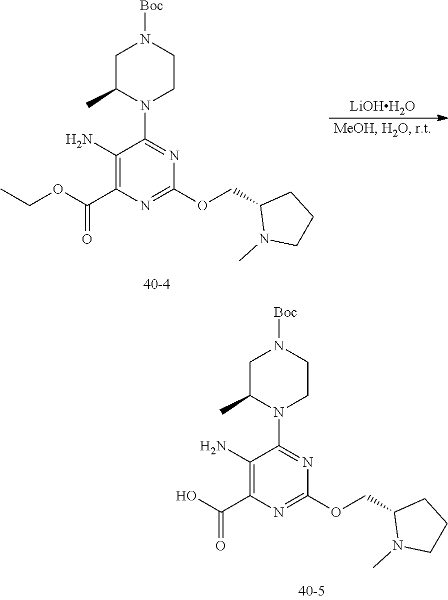 [SMILES: CCOC(=O)c1nc(OC[C@@H]2CCCN2C)nc(N2CCN(C(=O)OC(C)(C)C)C[C@@H]2C)c1N ; "LiOH·H2O" ; "MeOH, H2O, r.t." ; C[C@H]1CN(C(=O)OC(C)(C)C)CCN1c1nc(OC[C@@H]2CCCN2C)nc(C(=O)O)c1N]

To a mixture of ethyl 5-amino-6-((S)-4-(tert-butoxycarbonyl)-2-methylpiperazin-1-yl)-2-(((S)-1-methylpyrrolidin-2-yl) methoxy) pyrimidine-4-carboxylate (3.3 g, 0.007 mol, 1.0 eq.) in MeOH (60 mL) and H₂O (10 mL) was added LiOH·H₂O (1.45 g, 0.034 mol, 5.0 eq.). The mixture was stirred at room temperature for 2 h. LCMS showed starting material was consumed and desired product formed. The reaction mixture was acidified with aq. HCl (0.5 M) to pH=6, and then concentrated to dryness to obtain 5-amino-6-((S)-4-(tert-butoxycarbonyl)-2-methylpiperazin-1-yl)-2-(((S)-1-methylpyrrolidin-2-yl) methoxy) pyrimidine-4-carboxylic acid (5.06 g, crude, 40-5).

LCMS (ESI, m/z): [M+1]⁺=451; RT=0.928 min.

### Step 5: Synthesis of Compound 40-6

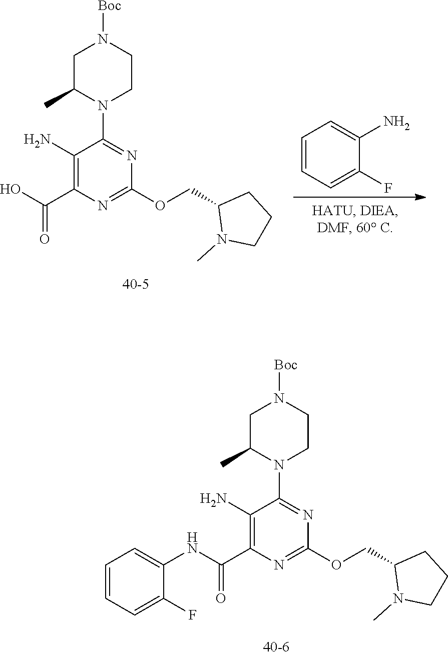 [SMILES: C[C@H]1CN(C(=O)OC(C)(C)C)CCN1c1nc(OC[C@@H]2CCCN2C)nc(C(=O)O)c1N ; Nc1ccccc1F ; C[C@H]1CN(C(=O)OC(C)(C)C)CCN1c1nc(OC[C@@H]2CCCN2C)nc(C(=O)Nc2ccccc2F)c1N]

To a solution of 5-amino-6-((S)-4-(tert-butoxycarbonyl)-2-methylpiperazin-1-yl)-2-(((S)-1-methylpyrrolidin-2-yl) methoxy) pyrimidine-4-carboxylic acid (1.00 g, 2.22 mmol, 1.0 eq.) and 2-fluoroaniline (197 mg, 1.77 mmol, 0.8 eq.) in anhydrous DMF (6.0 mL) was added DIEA (1.1 mL, 6.66 mmol, 3.0 eq.), followed by the addition of HATU (844 mg, 2.22 mmol, 1.0 eq.). The reaction mixture was stirred at 60° C. under Ar for 1 h. LCMS showed starting material was consumed and desired product formed. The reaction mixture was cooled to room temperature, diluted with water (30 mL) and extracted with EtOAc (15 mL×3). The combined organic fractions were washed with brine (20 mL), dried over anhydrous Na₂SO₄, filtered and concentrated. The residue was purified by silica column chromatography eluting with DCM/MeOH (10:1, v/v) to obtain tert-butyl (S)-4-(5-amino-6-((2-fluorophenyl)carbamoyl)-2-(((S)-1-methylpyrrolidin-2-yl)methoxy)pyrimidin-4-yl)-3-methylpiperazine-1-carboxylate (775 mg, 64% yield, 40-6).

LCMS (ESI, m/z): [M+1]⁺=544; RT=1.363 min.

### Step 6: Synthesis of Compound 40-7

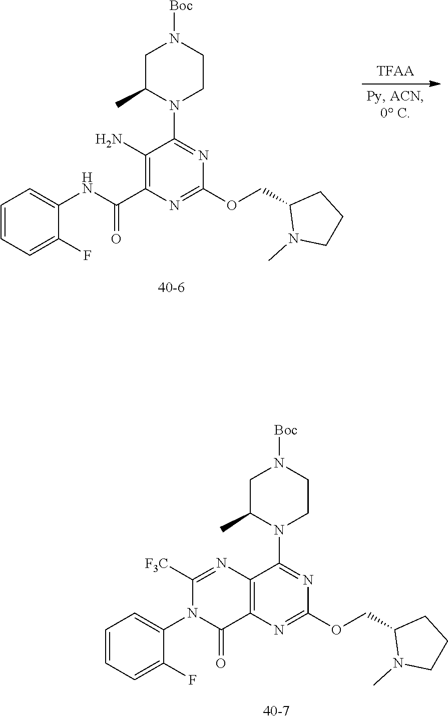 [SMILES: C[C@H]1CN(C(=O)OC(C)(C)C)CCN1c1nc(OC[C@@H]2CCCN2C)nc(C(=O)Nc2ccccc2F)c1N ; "TFAA" ; C[C@H]1CN(C(=O)OC(C)(C)C)CCN1c1nc(OC[C@@H]2CCCN2C)nc2c(=O)n(-c3ccccc3F)c(C(F)(F)F)nc12]

To a cooled (0° C.) solution of tert-butyl (S)-4-(5-amino-6-((2-fluorophenyl)carbamoyl)-2-(((S)-1-methylpyrrolidin-2-yl)methoxy)pyrimidin-4-yl)-3-methylpiperazine-1-carboxylate (395 mg, 0.726 mmol, 1.0 eq.) in anhydrous ACN (12 mL) was added pyridine (575 mg, 7.26 mmol, 10.0 eq.), followed by the addition of TFAA (916 mg, 4.36 mmol, 6.0 eq.). The mixture was stirred at 0° C. for 30 min. LCMS showed starting material was consumed and desired product formed. The reaction mixture was quenched with aq. NH₄Cl (sat. 40 mL) and extracted with EtOAc (20 mL×3). The combined organic fractions were washed with brine (30 mL), dried over anhydrous Na₂SO₄, filtered and concentrated obtain tert-butyl (S)-4-(7-(2-fluorophenyl)-2-(((S)-1-methylpyrrolidin-2-yl)methoxy)-8-oxo-6-(trifluoromethyl)-7,8-dihydropyrimido[5,4-d]pyrimidin-4-yl)-3-methylpiperazine-1-carboxylate (361 mg, crude, 40-7), which was used directly for the next step.

LCMS (ESI, m/z): [M+1]⁺=622; RT=1.462 min.

### Step 7: Synthesis of Compound 40-8

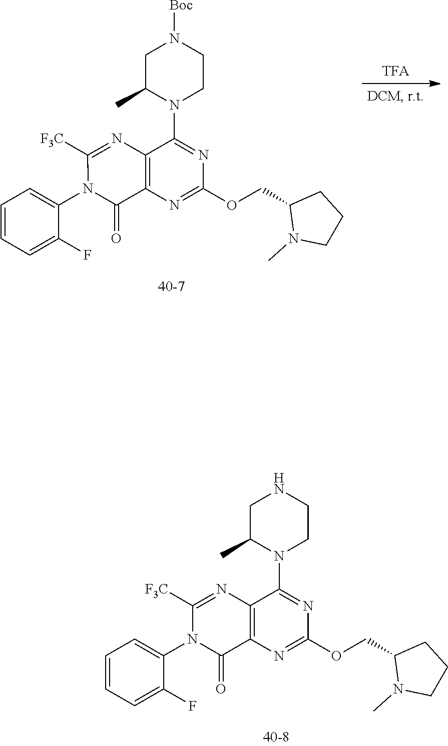 [SMILES: "TFA" ; C[C@H]1CN(C(=O)OC(C)(C)C)CCN1c1nc(OC[C@@H]2CCCN2C)nc2c(=O)n(-c3ccccc3F)c(C(F)(F)F)nc12 ; C[C@H]1CNCCN1c1nc(OC[C@@H]2CCCN2C)nc2c(=O)n(-c3ccccc3F)c(C(F)(F)F)nc12]

To a solution of tert-butyl (S)-4-(7-(2-fluorophenyl)-2-(((S)-1-methylpyrrolidin-2-yl)methoxy)-8-oxo-6-(trifluoromethyl)-7,8-dihydropyrimido[5,4-d]pyrimidin-4-yl)-3-methylpiperazine-1-carboxylate (361 mg, 0.581 mmol) in anhydrous DCM (6.0 mL) was added TFA (4 mL), and the mixture was stirred at room temperature for 2 h. LCMS showed starting material was consumed and desired product formed. The reaction mixture was basified with aq. NaHCO₃ (sat. 60 mL) to pH=7-8 and extracted with DCM (30 mL×2). The combined organic fractions were dried over anhydrous Na₂SO₄ and concentrated to dryness to obtain 3-(2-fluorophenyl)-8-((S)-2-methylpiperazin-1-yl)-6-(((S)-1-methylpyrrolidin-2-yl)methoxy)-2-(trifluoromethyl) pyrimido[5,4-d]pyrimidin-4(3H)-one (273 mg, crude, 40-8), which was used directly for the next step.

LCMS (ESI, m/z): [M+1]⁺=522; RT=0.930 min.

### Step 8: Synthesis of Compounds 40-a and 40-b

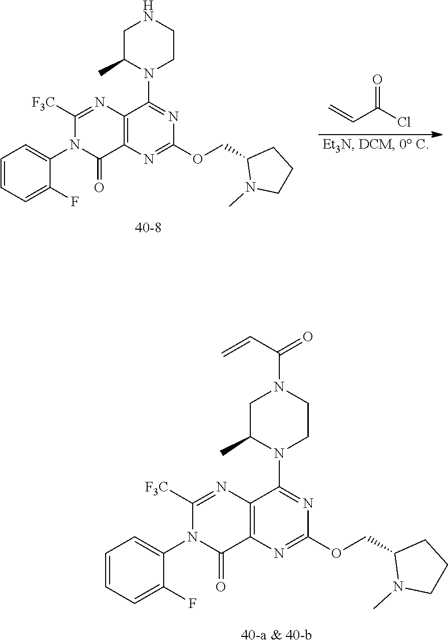

To a cooled (0° C.) solution of 3-(2-fluorophenyl)-8-((S)-2-methylpiperazin-1-yl)-6-(((S)-1-methylpyrrolidin-2-yl)methoxy)-2-(trifluoromethyl) pyrimido[5,4-d]pyrimidin-4(3H)-one (273 mg, 0.523 mmol, 1.0 eq.) and Et₃N (265 mg, 2.62 mmol, 5.0 eq.) in anhydrous DCM (10 mL) was added dropwise a solution of acryloyl chloride (62 mg, 0.681 mmol, 1.3 eq.) in anhydrous DCM (1 mL). After addition, the mixture was stirred at 0° C. for 30 min. LCMS showed starting material was consumed and desired product formed. Water (10 mL) was added and the organic layer was separated. The aqueous layer was extracted with DCM (5 mL×3). The combined organic fractions were dried over anhydrous Na₂SO₄ and concentrated. The residue was purified by prep-TLC eluting with DCM/MeOH (10:1, v/v) and then SFC to obtain 8-((S)-4-acryloyl-2-methylpiperazin-1-yl)-3-(2-fluorophenyl)-6-(((S)-1-methylpyrrolidin-2-yl)methoxy)-2-(trifluoromethyl)pyrimido[5,4-d]pyrimidin-4(3H)-one (4.57 mg, 1.5% yield, 40-a), and 8-((S)-4-acryloyl-2-methylpiperazin-1-yl)-3-(2-fluorophenyl)-6-(((S)-1-methylpyrrolidin-2-yl)methoxy)-2-(trifluoromethyl)pyrimido[5,4-d]pyrimidin-4(3H)-one (4.27 mg, 1.4% yield, 40-b).

40-a:

LCMS (ESI, m/z): [M+1]⁺=576; RT=1.975 min;

¹H NMR (400 MHz, CDCl₃) δ 7.53-7.44 (m, 1H), 7.28-7.21 (m, 3H), 6.53 (dd, J=26.5, 17.3 Hz, 1H), 6.32 (d, J=16.8 Hz, 1H), 5.71 (d, J=10.1 Hz, 1H), 5.28 (s, 1H), 4.92-4.12 (m, 3H), 4.01-3.71 (m, 1H), 3.52 (s, 2H), 3.43-3.00 (m, 2H), 2.89-2.27 (m, 4H), 2.16-1.90 (m, 3H), 1.61-1.50 (m, 2H), 1.33 (d, J=6.3 Hz, 4H);

¹⁹F NMR (400 MHz, CDCl₃) δ −65.7, −119.8.

40-b:

LCMS (ESI, m/z): [M+1]⁺=576; RT=1.977 min;

¹H NMR (400 MHz, CDCl₃) δ 7.58-7.52 (m, 1H), 7.34-7.28 (m, 3H), 6.67-6.52 (m, 1H), 6.39 (d, J=15.3 Hz, 1H), 5.78 (d, J=11.1 Hz, 1H), 5.30 (s, 1H), 4.72-4.23 (m, 3H), 4.09-3.74 (m, 1H), 3.66-3.49 (m, 2H), 3.45-2.99 (m, 2H), 2.92-2.20 (m, 5H), 2.05 (s, 1H), 1.79 (s, 3H), 1.40 (d, J=6.5 Hz, 4H);

¹⁹F NMR_ATG012-439-2 (400 MHz, CDCl₃) δ −65.7, −120.0.

### Example 40

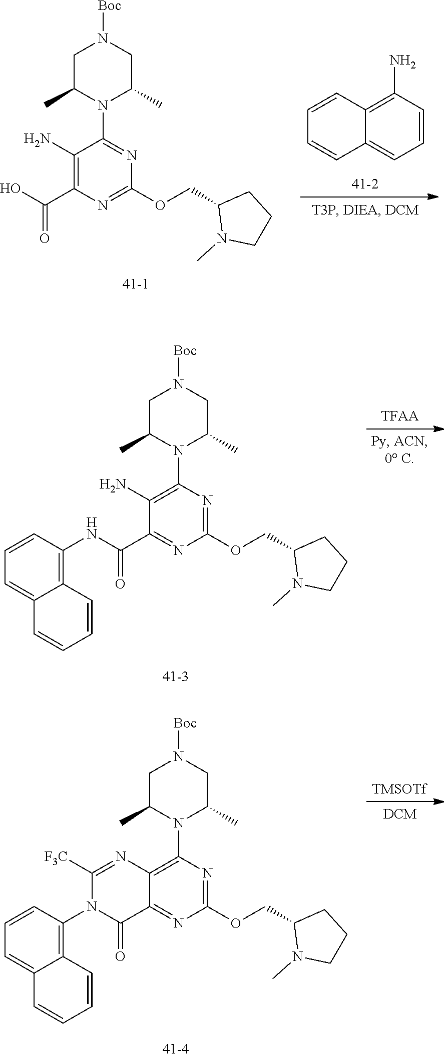 [SMILES: C[C@H]1CN(C(=O)OC(C)(C)C)C[C@H](C)N1c1nc(OC[C@@H]2CCCN2C)nc(C(=O)O)c1N ; Nc1cccc2ccccc12 ; C[C@H]1CN(C(=O)OC(C)(C)C)C[C@H](C)N1c1nc(OC[C@@H]2CCCN2C)nc(C(=O)Nc2cccc3ccccc23)c1N ; "TMSOTf" ; "TFAA" ; C[C@H]1CN(C(=O)OC(C)(C)C)C[C@H](C)N1c1nc(OC[C@@H]2CCCN2C)nc2c(=O)n(-c3cccc4ccccc34)c(C(F)(F)F)nc12]

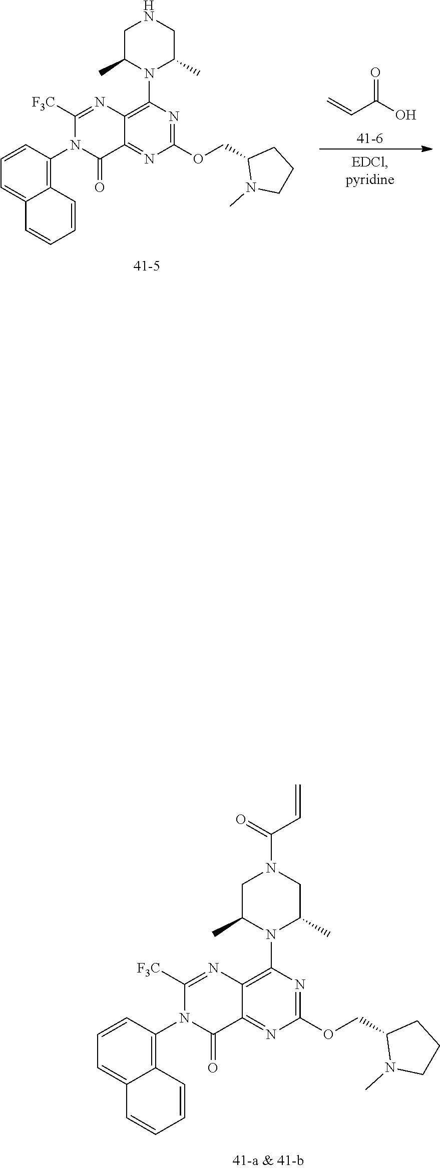

### Step 1: Synthesis of Compound 41-3

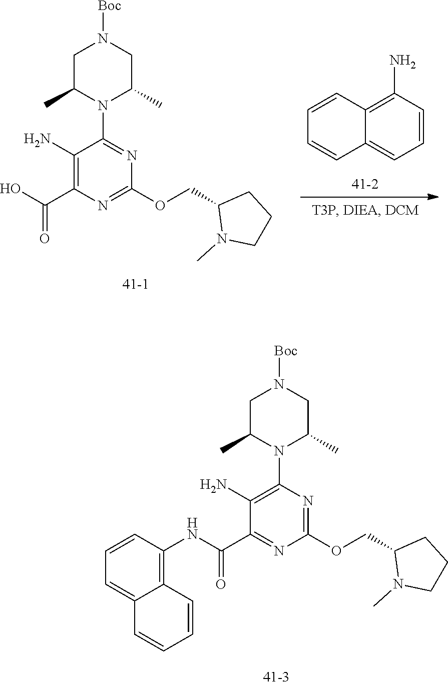 [SMILES: C[C@H]1CN(C(=O)OC(C)(C)C)C[C@H](C)N1c1nc(OC[C@@H]2CCCN2C)nc(C(=O)O)c1N ; Nc1cccc2ccccc12 ; C[C@H]1CN(C(=O)OC(C)(C)C)C[C@H](C)N1c1nc(OC[C@@H]2CCCN2C)nc(C(=O)Nc2cccc3ccccc23)c1N]

To a solution of compound 41-1 (500 mg, 1.08 mmol, 1.00 eq) in DCM (2.00 mL) was added T3P (3.42 g, 5.38 mmol, 3.20 mL, 50% purity, 5.00 eq) and compound 41-2 (185 mg, 1.29 mmol, 181 uL, 1.20 eq) at 20° C., then added DIEA (417 mg, 3.23 mmol, 562 uL, 3.00 eq) and the mixture was stirred at 20° C. for 2 hrs. LC-MS showed compound 41-1 was consumed completely and one main peak with desired mass was detected. The reaction mixture was diluted with H₂O (10.0 mL), extracted with EtOAc (10.0 mL×2), the organic layers were washed with H₂O (10.0 mL), brine (10.0 mL), dried over Na₂SO₄, filtrated and the filtrate was concentrated to give a residue. The residue was purified by flash silica gel chromatography (ISCO®; 20 g SepaFlash® Silica Flash Column, Eluent of 0˜10% MeOH/DCM @ 30 mL/min), TLC (Dichloromethane/Methanol=10/1, R_{f}=0.35). The residue was further purified by Prep-HPLC (column: Welch Ultimate XB-CN 250*70*10 um; mobile phase: [Heptane-EtOH]; B %: 1%-40%, 15 min). Compound 41-3 (510 mg, 865 umol, 80.3% yield) was obtained and confirmed by H NMR.

¹H NMR (400 MHz, DMSO-d₆): δ 10.69 (s, 1H), 8.04-7.93 (m, 2H), 7.89-7.82 (m, 2H), 7.62-7.54 (m, 3H), 6.18 (s, 2H), 4.44 (d, J=5.6 Hz, s, 2H), 3.77 (s, 3H), 3.42-3.27 (m, 4H), 3.17 (s, 2H), 2.05 (dd, J₁=2.0 Hz, J₂=5.6 Hz, 1H), 1.84-1.64 (m, 3H), 1.43 (s, 9H), 0.94 (d, J=5.6 Hz, 6H).

### Step 2. Synthesis of Compound 41-4

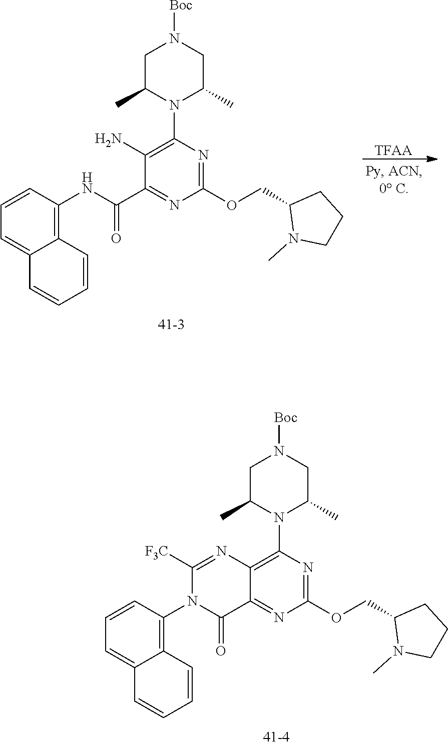 [SMILES: C[C@H]1CN(C(=O)OC(C)(C)C)C[C@H](C)N1c1nc(OC[C@@H]2CCCN2C)nc(C(=O)Nc2cccc3ccccc23)c1N ; "TFAA" ; C[C@H]1CN(C(=O)OC(C)(C)C)C[C@H](C)N1c1nc(OC[C@@H]2CCCN2C)nc2c(=O)n(-c3cccc4ccccc34)c(C(F)(F)F)nc12]

To a solution of compound 41-3 (400 mg, 678 umol, 1.00 eq) in ACN (20.0 mL) was added Py (536 mg, 6.78 mmol, 547 uL, 10.0 eq) at 0° C., then added and TFAA (855 mg, 4.07 mmol, 566 uL, 6.00 eq) at 0° C., then the mixture was stirred at 0° C. for 10.0 mins. TLC (SiO₂, DCM/MeOH=10/1, R_{f}=0.45) indicated compound 41-3 was consumed completely and one new spot formed. The reaction mixture was diluted with sat.aq NH₄Cl (10.0 mL), extracted with EtOAc (10.0 mL×2), the organic layers were washed with H₂O (10.0 mL), brine (10.0 mL), dried over Na₂SO₄, filtrated and the filtrate was concentrated to give a residue. Compound 41-4 (400 mg, crude) was obtained.

### Step 3. Synthesis of Compound 41-5

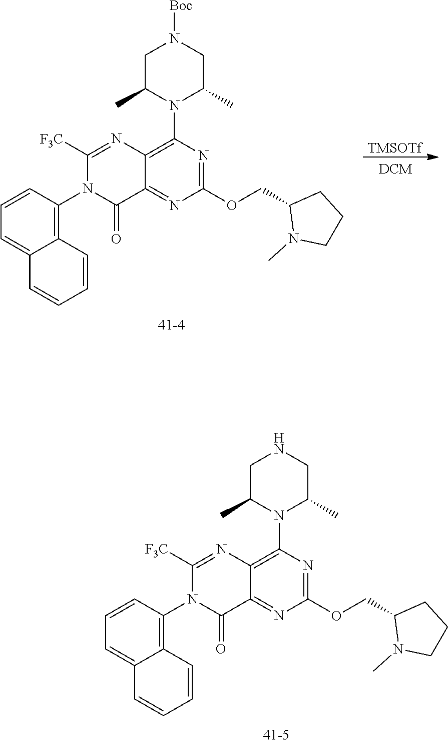 [SMILES: "TMSOTf" ; C[C@H]1CN(C(=O)OC(C)(C)C)C[C@H](C)N1c1nc(OC[C@@H]2CCCN2C)nc2c(=O)n(-c3cccc4ccccc34)c(C(F)(F)F)nc12 ; C[C@H]1CNC[C@H](C)N1c1nc(OC[C@@H]2CCCN2C)nc2c(=O)n(-c3cccc4ccccc34)c(C(F)(F)F)nc12]

To a solution of compound 41-4 (400 mg, 599 umol, 1.00 eq) in DCM (5.00 mL) was added TMSOTf (266 mg, 1.20 mmol, 216 uL, 2.00 eq) at 0° C., then the mixture was stirred at 0° C. for 30 mins. LC-MS showed compound 41-4 was consumed completely and one main peak with desired mass was detected. The mixture was adjusted to pH=8 with saturated aqueous NaHCO₃, diluted with H₂O (10.0 mL), extracted with DCM (10.0 mL×2), the organic layers were washed with H₂O (10.0 mL), brine (10.0 mL), dried over Na₂SO₄, filtrated and the filtrate was concentrated to give a residue. Compound 41-5 (400 mg, crude) was obtained.

### Step 4. Synthesis of Compounds 41-a and 41-b

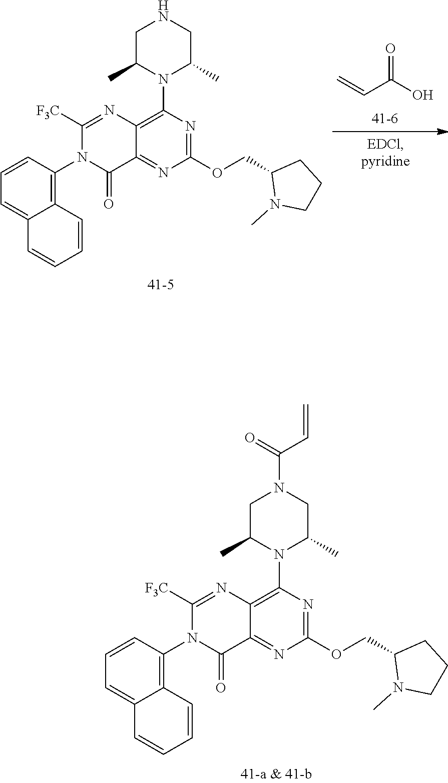

To a solution of compound 41-5 (400 mg, 704 umol, 1.00 eq) and compound 41-6 (101 mg, 1.41 mmol, 96.7 uL, 2.00 eq) in pyridine (5.00 mL) was added EDCI (540 mg, 2.82 mmol, 4.00 eq) at 0° C., then the mixture was stirred at 0° C. for 20 mins. LC-MS showed compound 41-5 was consumed completely and one main peak with desired mass was detected. The reaction mixture was diluted with H₂O (10.0 mL), extracted with DCM (10.0 mL×2), the organic layers were washed with H₂O (10.0 mL), brine (10.0 mL), dried over Na₂SO₄, filtrated and the filtrate was concentrated to give a residue. The residue was purified by Prep-HPLC (column: Unisil 3-100 C₁₈ Ultra 150*50 mm*3 um; mobile phase: [water (FA)−ACN]; B %: 18%-48%, 10 min). Then the mixture was further purified by SFC separation (column: DAICEL CHIRALCEL OD (250 mm*30 mm, 10 um); mobile phase: [0.1% NH₃H₂O MEOH]; B %: 45%-45%, 4.0 min; 20 minmin). 41-a (6.00 mg, 9.61 umol, 1.36% yield, 99.5% purity) and 41-b (20.0 mg, 31.2 umol, 4.44% yield, 97.2% purity) were obtained.

41-a:

LC-MS: RT=0.838 min, (M+H)⁺=622.5

1H NMR (400 MHz, DMSO-d₆): δ 8.15 (d, J=7.5 Hz, 1H), 8.08 (d, J=8.0 Hz, 1H), 7.82 (d, J=8.4 Hz, 1H), 7.73-7.65 (m, 2H), 7.64-7.59 (m, 1H), 7.58-7.52 (m, 1H), 6.79 (dd, J₁=10.4 Hz, J₂=16.8 Hz, 1H), 6.22 (dd, J₁=2.0 Hz, J₂=16.4 Hz, 1H), 5.80-5.74 (m, 1H), 5.50-5.23 (m, 1H), 4.39 (dd, J₁=5.2 Hz, J₂=10.8 Hz, 1H), 4.22 (dd, J₁=5.6 Hz, J₂=10.0 Hz, 1H), 4.09-3.94 (m, 3H), 3.66 (dd, J₁=3.6 Hz, J₂=14.4 Hz, 1H), 2.97 (s, 1H), 2.66-2.57 (m, 1H), 2.37 (s, 3H), 2.26-2.15 (m, 1H), 2.01-1.90 (m, 1H), 1.72-1.56 (m, 3H), 1.41 (t, J=6.4 Hz, 6H), 1.23 (s, 1H).

HPLC: 99.5% purity

SFC: RT=1.894 min, 100% ee

41-b:

¹H NMR (400 MHz, DMSO-d₆): δ 8.16 (d, J=8.0 Hz, 1H), 8.08 (d, J=8.0 Hz, 1H), 7.78 (d, J=7.2 Hz, 1H), 7.75-7.66 (m, 2H), 7.64-7.58 (m, 1H), 7.56-7.49 (m, 1H), 6.79 (dd, J₁=10.4 Hz, J₂=16.8 Hz, 1H), 6.22 (dd, J₁=2.4 Hz, J₂=16.8 Hz, 1H), 5.80-5.73 (m, 1H), 5.50-5.24 (m, 1H), 4.40 (dd, J₁=5.2 Hz, J₂=10.8 Hz, 1H), 4.19 (dd, J₁=6.0 Hz, J₂=10.8 Hz, 1H), 4.07-3.94 (m, 3H), 3.65 (dd, J₁=3.6 Hz, J₂=14.4 Hz, 1H), 3.01-2.90 (m, 1H), 2.60 (s, 1H), 2.36 (s, 3H), 2.25-2.15 (m, 1H), 2.01-1.89 (m, 1H), 1.74-1.57 (m, 3H), 1.42 (t, J=5.6 Hz, 6H), 1.23 (s, 1H).

LC-MS: RT=0.839 min, (M+H)⁺=622.5

HPLC: 97.2% purity

SFC: RT=2.335 min, 100% ee

### Example 41

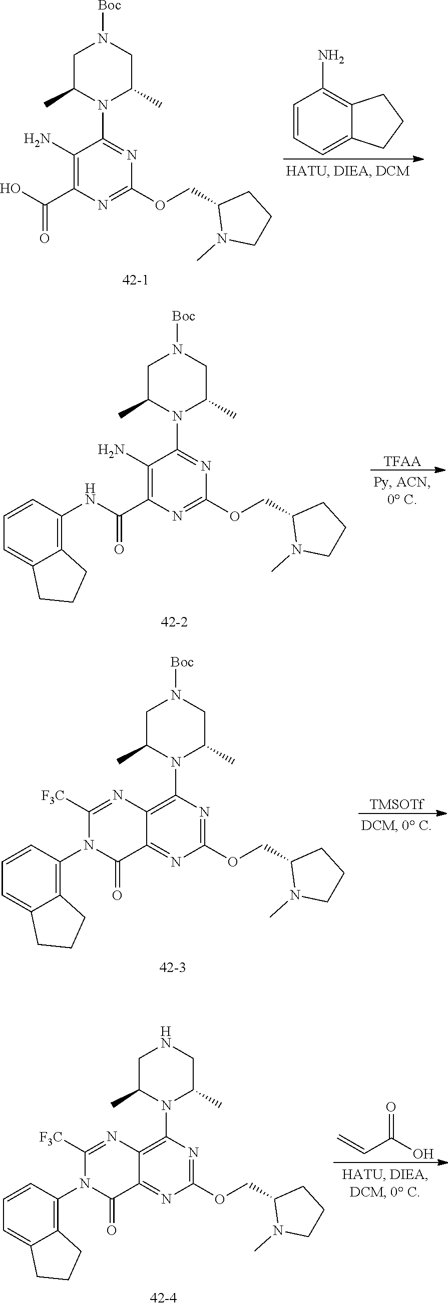

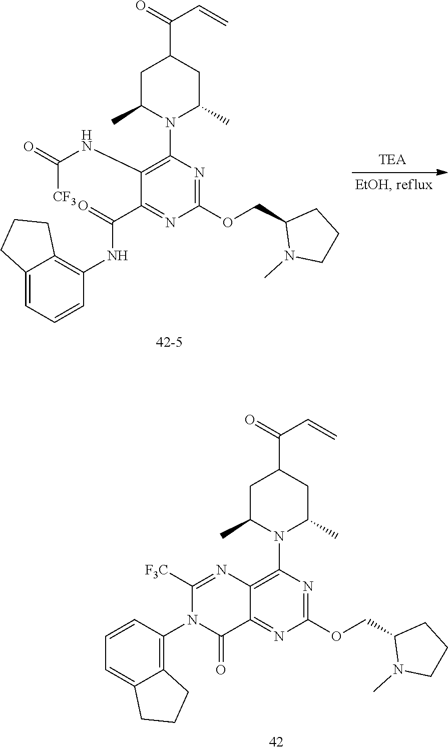 [SMILES: C=CC(=O)C1C[C@H](C)N(c2nc(OC[C@H]3CCCN3C)nc(C(=O)Nc3cccc4c3CCC4)c2NC(=O)C(F)(F)F)[C@@H](C)C1 ; C=CC(=O)C1C[C@H](C)N(c2nc(OC[C@@H]3CCCN3C)nc3c(=O)n(-c4cccc5c4CCC5)c(C(F)(F)F)nc23)[C@@H](C)C1]

### Step 1: Synthesis of Compound 42-2

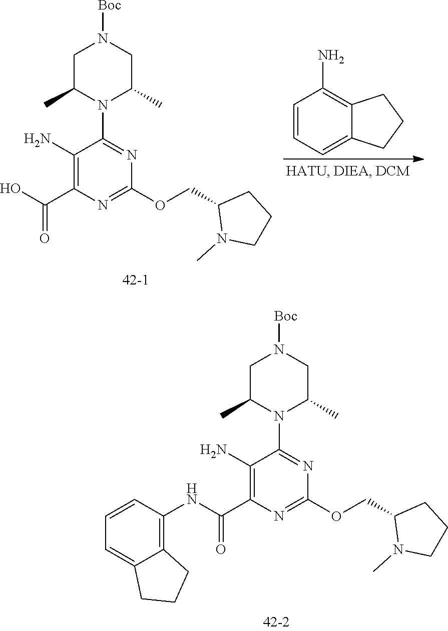 [SMILES: C[C@H]1CN(C(=O)OC(C)(C)C)C[C@H](C)N1c1nc(OC[C@@H]2CCCN2C)nc(C(=O)O)c1N ; Nc1cccc2c1CCC2 ; C[C@H]1CN(C(=O)OC(C)(C)C)C[C@H](C)N1c1nc(OC[C@@H]2CCCN2C)nc(C(=O)Nc2cccc3c2CCC3)c1N]

To a solution of compound 42-1 (400 mg, 0.86 mmol) and 2,3-dihydro-1H-inden-4-amine (137.62 mg, 1.03 mmol) in DCM (10.0 mL) was added DIEA (333.85 mg, 2.58 mmol) and HATU (491.08 mg, 1.29 mmol). The reaction mixture was stirred at 20° C. for 12 h. The reaction mixture was quenched by water (10.0 mL), and then diluted with DCM (15.0 mL) and extracted with DCM (15.0 mL×2). The combined organic layers were washed with brine (20.0 mL), dried over Na₂SO₄, filtered and concentrated under reduced pressure to give a residue. The residue was purified by silica column chromatography eluting with DCM/MeOH (10:1, v/v) to obtain 42-2 (365 mg, 73.1% yield).

### Step 2: Synthesis of Compound 42-3

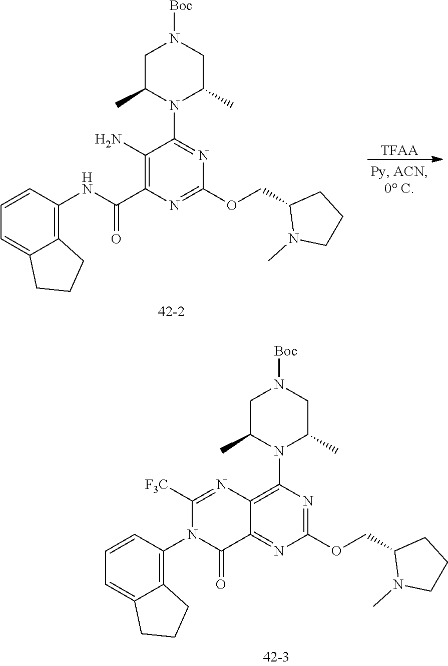 [SMILES: C[C@H]1CN(C(=O)OC(C)(C)C)C[C@H](C)N1c1nc(OC[C@@H]2CCCN2C)nc(C(=O)Nc2cccc3c2CCC3)c1N ; "TFAA" ; C[C@H]1CN(C(=O)OC(C)(C)C)C[C@H](C)N1c1nc(OC[C@@H]2CCCN2C)nc2c(=O)n(-c3cccc4c3CCC4)c(C(F)(F)F)nc12]

To a solution of compound 42-2 (365 mg, 0.63 mmol) in ACN (35 mL) was added Py (498 mg, 6.30 mmol) and TFAA (793.4 mg, 3.78 mmol) at 0° C. Then the mixture was stirred at 0° C. for 15 min. TLC (DCM/MeOH=10/1, R_{f}=0.45) indicated compound 42-2 was consumed completely, and one major new spot with larger polarity was detected. The reaction mixture was quenched by addition saturated NH₄Cl (15 mL) aqueous solution at 0° C., and then diluted with EtOAc (15 mL) and extracted with solvent EtOAc (20 mL×3). The combined organic layers were dried over anhydrous Na₂SO₄, filtered and concentrated under reduced pressure to give a residue 42-3 (512 mg, crude). The crude product was used into the next step without further purification.

### Step 3: Synthesis of Compound 42-4

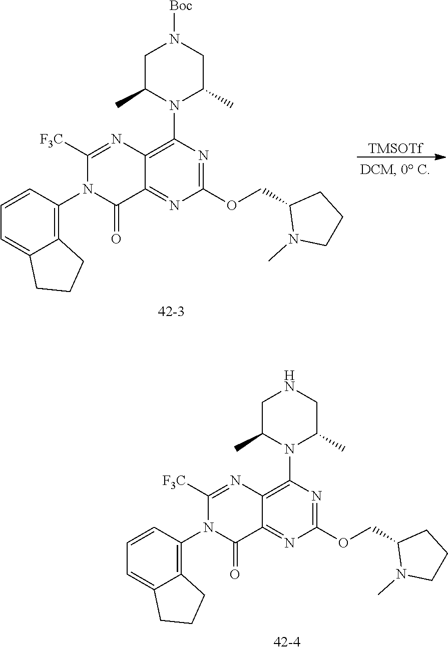 [SMILES: "TMSOTf" ; C[C@H]1CN(C(=O)OC(C)(C)C)C[C@H](C)N1c1nc(OC[C@@H]2CCCN2C)nc2c(=O)n(-c3cccc4c3CCC4)c(C(F)(F)F)nc12 ; C[C@H]1CNC[C@H](C)N1c1nc(OC[C@@H]2CCCN2C)nc2c(=O)n(-c3cccc4c3CCC4)c(C(F)(F)F)nc12]

To a solution of compound 42-3 (512 mg, 0.78 mmol) in DCM (10.0 mL) was added TMSOTf (259.51 mg, 1.17 mmol) at 0° C. Then the mixture was stirred at 0° C. for 15 min. TLC (DCM/MeOH=10/1, R_{f}=0.1) showed compound 42-3 was consumed completely and one main peak with desired mass was detected. The reaction mixture was quenched by addition saturated NaHCO₃ (10.0 mL) aqueous solution at 0° C., and then diluted with DCM (10.0 mL) and extracted with solvent DCM (10 mL×2). The combined organic layers were dried over anhydrous Na₂SO₄, filtered and concentrated under reduced pressure to give a residue 42-4 (520 mg, crude). The crude product was used into the next step without further purification.

### Step 4: Synthesis of Compound 42-5

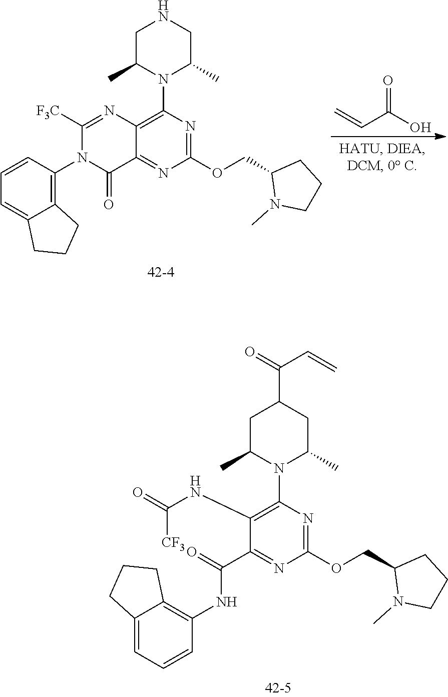

To a solution of compound 42-4 (520 mg, 0.93 mmol) in DCM (10.0 mL) was added propionic acid (89.81 mg, 1.21 mmol), DIEA (361.58 mg, 2.80 mmol) and HATU (531.87 mg, 1.40 mmol) at 0° C. Then the mixture was stirred at 0° C. for 30 min. TLC (DCM/MeOH=10/1, R_{f}=0.3) showed compound 42-4 was consumed completely. Several new spots were shown on TLC. The reaction mixture was quenched by addition saturated NH₄Cl (8.0 mL) aqueous solution at 0° C., and then diluted with DCM (10.0 mL) and extracted with solvent DCM (10.0 mL×3). The combined organic layers were dried over anhydrous Na₂SO₄, filtered and concentrated under reduced pressure to give a residue. The residue was purified by pre-TLC (DCM/MeOH=10/1, R_{f}=0.3) to afford the desired product 42-5 (70 mg, 11.9% yield).

### Step 5: Synthesis of Compound 42

To a solution of compound 42-5 (70 mg, 0.11 mmol) in EtOH (7.0 mL) was added TEA (247.48 mg, 2.45 mmol). Then the mixture was stirred at 88° C. for 12 h. TLC (DCM/MeOH=10/1, R_{f}=0.4) showed compound 42-5 was consumed completely. The reaction mixture was cooled to 20° C., H₂O (10 mL) was added to the mixture, the mixture diluted with DCM (15.0 mL) and extracted with solvent DCM (10.0 mL×3). The combined organic layers were dried over anhydrous Na₂SO₄, filtered and concentrated under reduced pressure to give a residue. The residue was purified by pre-TLC (DCM/MeOH=10/1, R_{f}=0.4) to afford the desired product 42 (4 mg, 5.9% yield). m/z: 612.1 [M+H]⁺. ¹H NMR (400 MHz, DMSO-d₆) δ ppm 8.06 (s, 1H) 7.40 (s, 1H) 6.81-6.83 (br d, J=1.76 Hz, 1H) 6.63 (d, J=1.76 Hz, 1H) 6.23 (d, J=2.02 Hz, 1H) 5.78 (d, J=2.26 Hz, 1H) 4.65 (s, 1H) 4.36-4.40 (m, 2H) 4.18-4.22 (m, 2H) 4.00 (br d, J=2.76 Hz, 2H) 3.63-3.65 (m, 2H) 2.98-3.01 (m, 4H) 2.44 (br d, J=3.26 Hz, 1H) 2.30-2.34 (m, 1H) 2.12 (s, 3H) 1.80-1.87 (m, 2H) 1.66-1.70 (m, 4H) 1.33-1.34 (m, 6H).

### Example 42

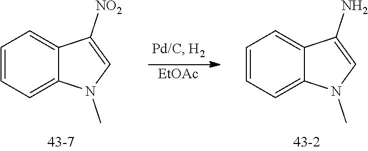

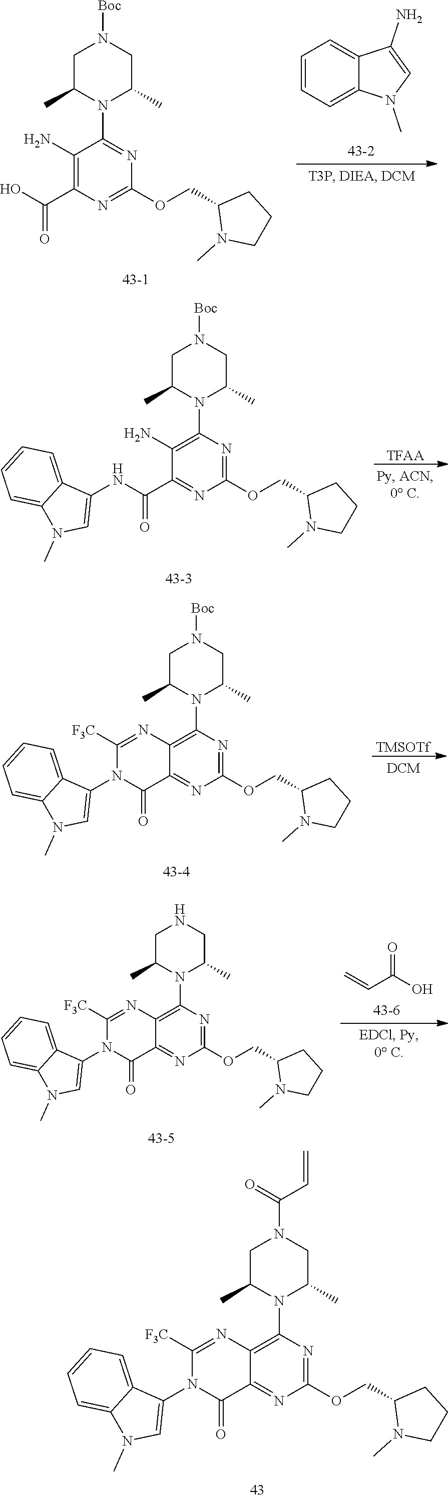

### Step 1. Synthesis of Compound 43-2

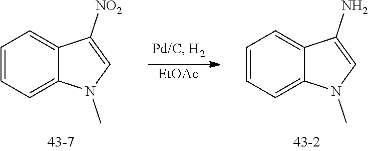

To a solution of compound 7 (100 mg, 568 umol, 1.00 eq) in Ethyl Acetate (10.0 mL) was added Pd/C (20.0 mg, 568 umol, 10.0% purity, 1.00 eq) under N₂, the reaction mixture was stirred at 20° C. for 3 hrs under H₂ (15 psi). LC-MS showed compound 43-7 was consumed completely and desired mass was detected. The reaction mixture was filtered and washed with Ethyl Acetate (100 mL). The filtrate was concentrated under reduced pressure to give a residue. Compound 43-2 (82.88 mg, crude) was obtained.

### Step 2. Synthesis of Compound 43-3

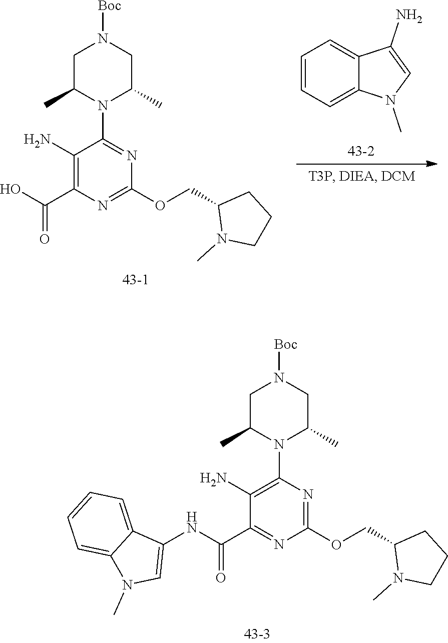 [SMILES: C[C@H]1CN(C(=O)OC(C)(C)C)C[C@H](C)N1c1nc(OC[C@@H]2CCCN2C)nc(C(=O)O)c1N ; Cn1cc(N)c2ccccc21 ; C[C@H]1CN(C(=O)OC(C)(C)C)C[C@H](C)N1c1nc(OC[C@@H]2CCCN2C)nc(C(=O)Nc2cn(C)c3ccccc23)c1N]

To a solution of compound 43-1 (1.32 g, 2.83 mmol, 1.00 eq) in DCM (5.00 mL) was added T3P (3.60 g, 5.66 mmol, 3.37 mL, 50.0% purity, 2.00 eq), DIEA (1.46 g, 11.3 mmol, 1.97 mL, 4.00 eq) and compound 43-2 (414 mg, 2.83 mmol, 1.00 eq). The mixture was stirred at 25° C. for 1 hr. LC-MS (EW29115-57-P1A1, product: RT=0.900 min) and HPLC (EW29115-57-P1A3) showed compound 43-1 was consumed completely and desired mass was detected. The reaction mixture was diluted with H₂O 100 mL and extracted with DCM 75.0 mL (25.0 mL×3). The combined organic layers were dried over Na₂SO₄, filtered and concentrated under reduced pressure to give a residue. The residue was purified by prep-HPLC (column: Welch Ultimate XB-CN 250*70*10 um; mobile phase: [Hexane-EtOH (0.1% NH₃.H₂O)]; B %: 10%-50%, 15 min). Compound 43-3 (1.10 g, 1.86 mmol, 65.5% yield) was obtained.

¹H NMR (400 MHz, CDCl₃): δ 10.0 (s, 1H), 7.81 (s, 1H), 7.65 (d, J=7.6 Hz, 1H), 7.35-7.28 (m, 2H), 7.19-7.15 (m, 1H), 5.72 (s, 2H), 4.45-4.39 (m, 1H), 4.32-4.26 (m, 1H), 3.89-3.88 (m, 2H), 3.82 (s, 3H), 3.59-3.29 (m, 3H), 3.17-3.13 (m, 1H), 2.81-2.75 (m, 1H), 2.55 (s, 3H), 2.38-2.34 (m, 1H), 2.15-2.11 (m, 1H), 1.90-1.75 (m, 4H), 1.50 (s, 9H), 1.02 (d, J=6.4 Hz, 6H)

SFC: RT=1.427 min, 100% ee

### Step 3. Synthesis of Compound 43-4

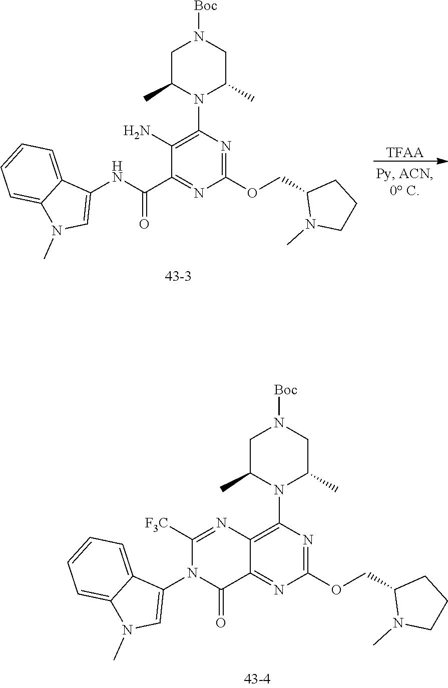 [SMILES: C[C@H]1CN(C(=O)OC(C)(C)C)C[C@H](C)N1c1nc(OC[C@@H]2CCCN2C)nc(C(=O)Nc2cn(C)c3ccccc23)c1N ; "TFAA" ; C[C@H]1CN(C(=O)OC(C)(C)C)C[C@H](C)N1c1nc(OC[C@@H]2CCCN2C)nc2c(=O)n(-c3cn(C)c4ccccc34)c(C(F)(F)F)nc12]

To a solution of compound 43-3 (300 mg, 506 umol, 1.00 eq) in anhydrous ACN (15.0 mL) was added Py (400 mg, 5.06 mmol, 409 uL, 10.0 eq), followed by the addition of TFAA (638 mg, 3.04 mmol, 422 uL, 6.00 eq). The mixture was stirred at 0° C. for 10 mins. LC-MS showed compound 43-3 was consumed completely and one main peak with desired mass was detected. The reaction mixture was quenched by addition saturated ammonium chloride aqueous solution 200 mL at 0° C., and extracted with Ethyl Acetate 75.0 mL (25.0 mL×3). The combined organic layers were dried over Na₂SO₄, filtered and concentrated under reduced pressure to give a residue. Compound 43-4 (330 mg, crude) was obtained.

### Step 4. Synthesis of Compound 43-5

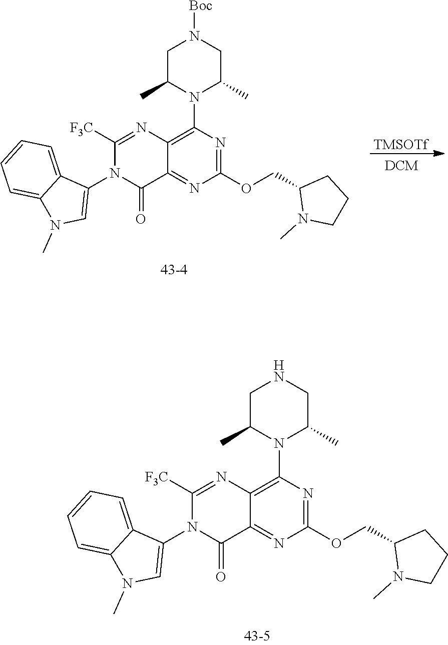

To a solution of compound 43-4 (330 mg, 492 umol, 1.00 eq) in DCM (15.0 mL) was added TMSOTf (219 mg, 984 umol, 178 uL, 2.00 eq). The mixture was stirred at 0° C. for 10 mins. LC-MS showed compound 43-4 was consumed completely and desired mass was detected. The reaction mixture was quenched by addition saturated Sodium bicarbonate solution 50.0 mL at 0° C., and then diluted with H₂O 100 mL and extracted with DCM 75.0 mL (25.0 mL×3). The combined organic layers were dried over Na₂SO₄, filtered and concentrated under reduced pressure to give a residue. Compound 43-5 (250 mg, crude) was obtained.

### Step 5. Synthesis of Compound 43

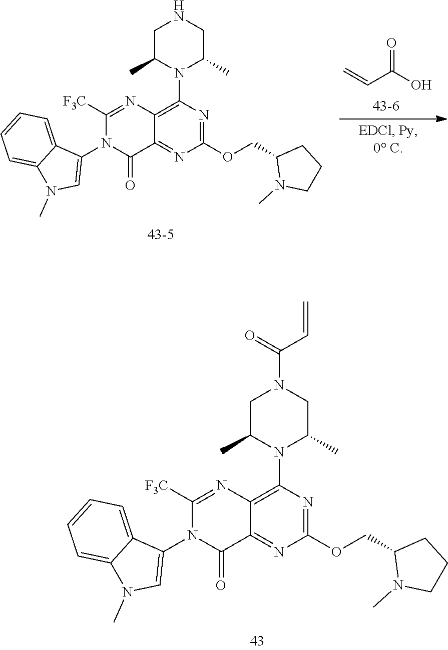

To a solution of compound 43-5 (250 mg, 438 umol, 1.00 eq), acrylic acid (63.2 mg, 876 umol, 60.1 uL, 2.00 eq) in Py (3.00 mL) was added EDCI (336 mg, 1.75 mmol, 4.00 eq). The mixture was stirred at 0° C. for 10 mins. LC-MS showed compound 43-5 was consumed completely and desired mass was detected. The reaction mixture was quenched by addition Saturated ammonium chloride aqueous solution 150 mL at 0° C., and then diluted with H₂O 100 mL and extracted with DCM 75.0 mL (25.0 mL*3). The combined organic layers were dried over Na₂SO₄, filtered and concentrated under reduced pressure to give a residue. The residue was purified by prep-HPLC (column: Welch Ultimate XB-CN 250*25*10 um; mobile phase: [Heptane-EtOH (0.1% NH₃H₂O)]; B %: 35%-75%, 15 min). 43 (10.0 mg, crude) was obtained and was purified by Prep-TLC (SiO2, DCM/MeOH=10/1, Plate 1, DCM/MeOH=10/1, R_{f}=0.34) to yield 43 (1.52 mg, 2.24 umol, 14.01% yield, 92.2% purity).

LC-MS: RT=0.842 min, (M+H)⁺=625.1

### Example 43

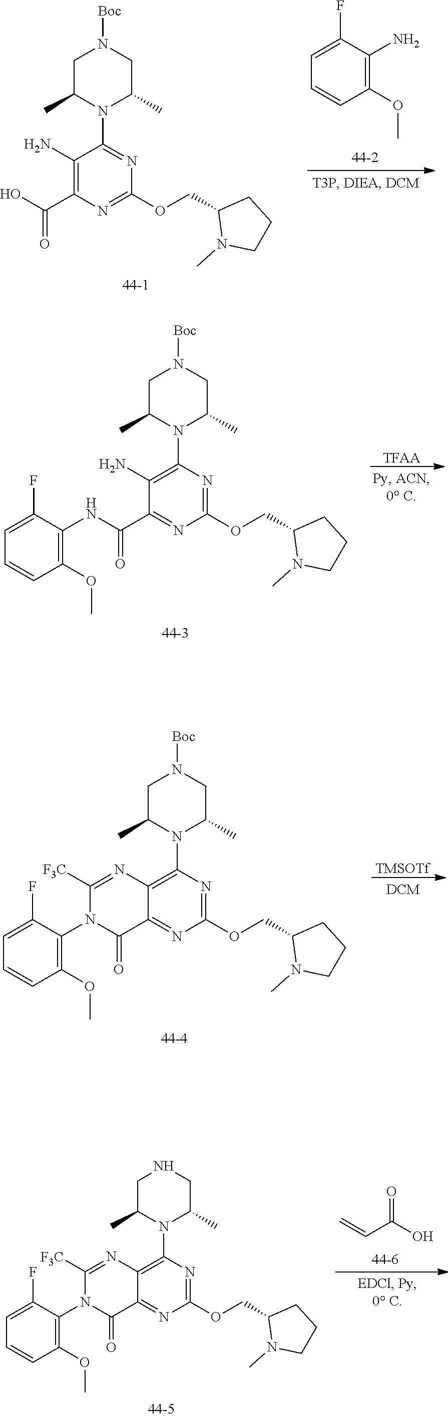

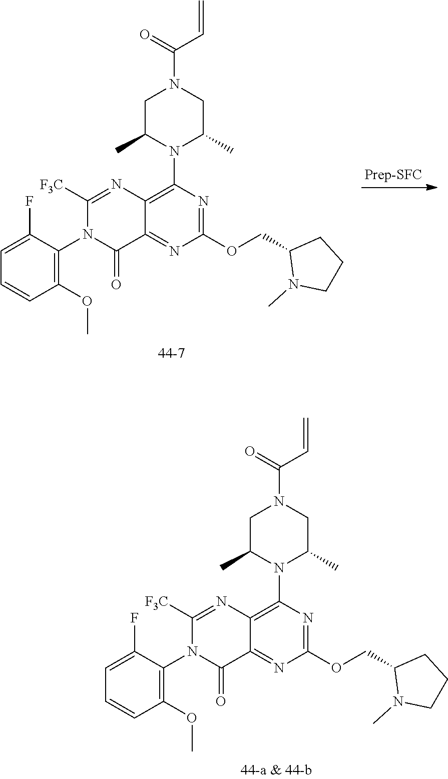 [SMILES: C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1 ; C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1]

### Step 1: Synthesis of Compound 44-3

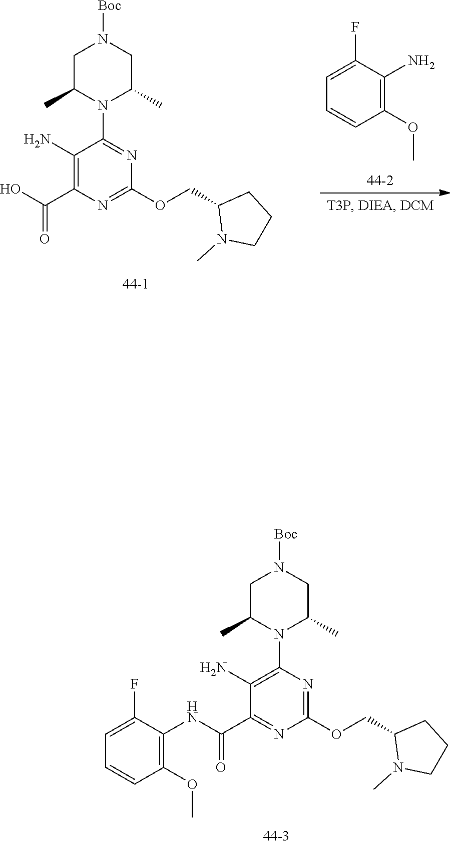

To a solution of compound 1 (500 mg, 1.08 mmol, 1.00 eq), compound 2 in DCM (5.00 mL) was added DIEA (417 mg, 3.23 mmol, 562 uL, 3.00 eq) and T3P (3.42 g, 5.38 mmol, 3.20 mL, 50.0% purity, 5.00 eq). The mixture was stirred at 20° C. for 2 hr. LC-MS (EW28938-58-P1A, product: RT=0.779 min) showed compound 1 was consumed completely and desired mass was detected. The reaction mixture was diluted with H₂O (30.0 mL), extracted with DCM (30.0 mL×3), the organic layers were washed with H₂O (30.0 mL), brine (30.0 mL), dried over Na₂SO₄, filtrated and the filtrate was concentrated to give a residue. The combined residue (EW28938-55 and EW28938-58) was purified by column chromatography (SiO₂, DCM/MeOH=10/1, Plate 1, DCM/MeOH=10/1, R_{f}=0.4). Compound 3 (565 mg, 961 umol, 89.3% yield, 100% purity) was obtained.

¹H NMR (400 MHz, DMSO-d₆): δ 9.77 (s, 1H), 7.37-7.31 (m, 1H), 6.98-6.89 (m, 2H), 6.17 (s, 2H), 4.70-4.57 (m, 2H), 3.81-3.44 (m, 11H), 2.85 (s, 3H), 2.24-2.20 (m, 1H), 1.99-1.78 (m, 4H), 1.42 (s, 9H), 0.92 (d, J=5.6 Hz, 6H).

SFC: RT=0.993 min, 100% ee

### Step 2: Synthesis of Compound 44-4

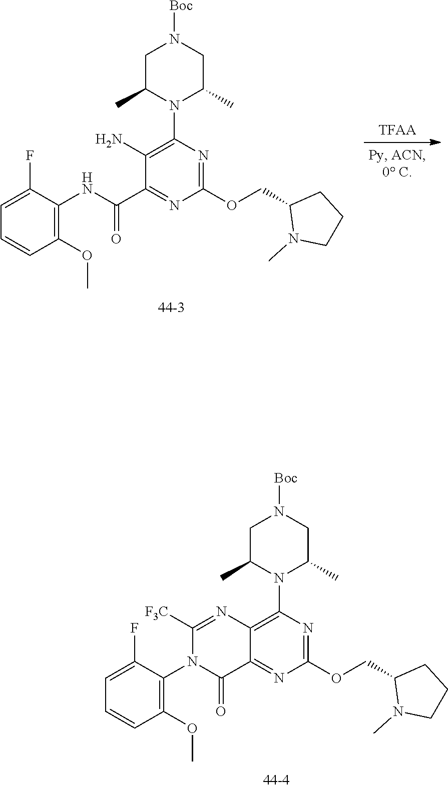 [SMILES: COc1cccc(F)c1NC(=O)c1nc(OC[C@@H]2CCCN2C)nc(N2[C@@H](C)CN(C(=O)OC(C)(C)C)C[C@@H]2C)c1N ; "TFAA" ; COc1cccc(F)c1-n1c(C(F)(F)F)nc2c(N3[C@@H](C)CN(C(=O)OC(C)(C)C)C[C@@H]3C)nc(OC[C@@H]3CCCN3C)nc2c1=O]

To a solution of compound 44-3 (400 mg, 681 umol, 100% purity, 1.00 eq) in ACN (20.0 mL) was added Py (538 mg, 6.81 mmol, 549 uL, 10.0 eq), followed by the addition of TFAA (858 mg, 4.08 mmol, 568 uL, 6 eq). The mixture was stirred at 0° C. for 10 min. LC-MS showed compound 44-3 was consumed completely and desired mass was detected. The reaction mixture was quenched with aq. NH₄Cl (sat. 40.0 mL×3) and extracted with EtOAc (30.0 mL×3). The combined organic fractions were washed with brine (40.0 mL), dried over Na₂SO₄, filtered and concentrated under reduced pressure to give a residue. The crude product was used into the next step without further purification. Compound 44-4 (500 mg, crude) was obtained.

### Step 3: Synthesis of Compound 44-5

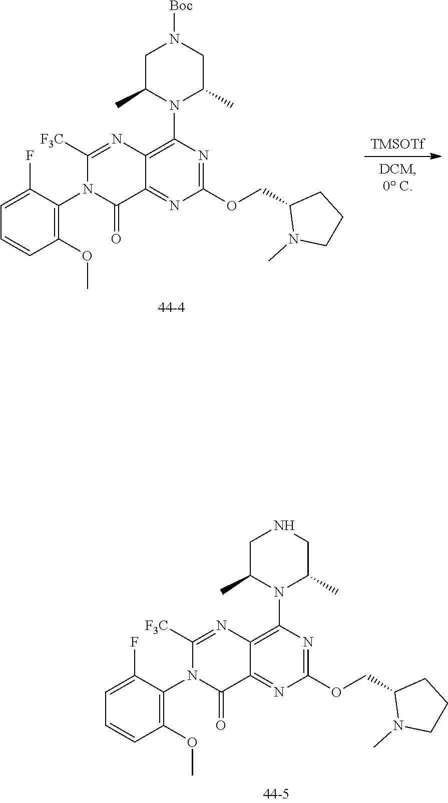 [SMILES: "TMSOTf" ; COc1cccc(F)c1-n1c(C(F)(F)F)nc2c(N3[C@@H](C)CN(C(=O)OC(C)(C)C)C[C@@H]3C)nc(OC[C@@H]3CCCN3C)nc2c1=O ; COc1cccc(F)c1-n1c(C(F)(F)F)nc2c(N3[C@@H](C)CNC[C@@H]3C)nc(OC[C@@H]3CCCN3C)nc2c1=O]

To a solution of Compound 44-4 (500 mg, 751 umol, 1.00 eq) in DCM (40.0 mL) was added TMSOTf (334 mg, 1.50 mmol, 271 uL, 2.00 eq). The mixture was stirred at 0° C. for 1 hr. LC-MS showed Compound 44-4 was consumed completely and desired mass was detected. The reaction mixture was quenched with aq.NaHCO₃ (sat.15.0 mL×3) and extracted with DCM (20.0 mL×3). The combined organic fractions were washed with brine (40.0 mL), dried over Na₂SO₄, filtered and concentrated under reduced pressure to give a residue. The crude product was used into the next step without further purification. Compound 44-5 (300 mg, crude) was obtained.

### Step 4: Synthesis of Compound 44-7

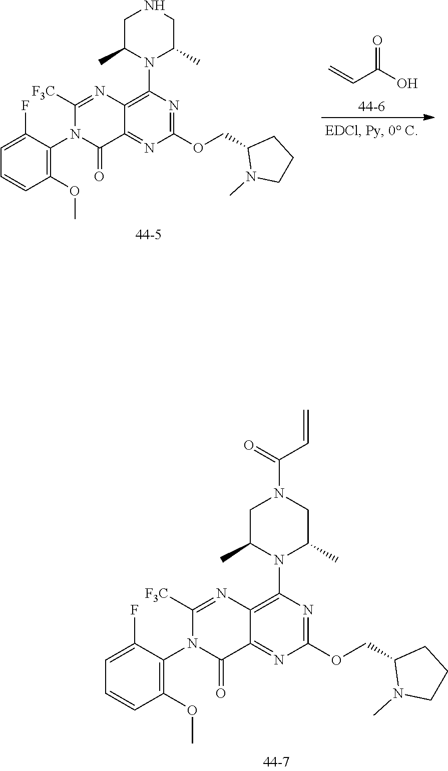

To a solution of compound 44-5 (250 mg, 442 umol, 1.00 eq), compound 44-6 (63.7 mg, 884 umol, 60.7 uL, 2.00 eq) in Py (4.00 mL) was added EDCI (127 mg, 663 umol, 1.50 eq). The mixture was stirred at 0° C. for 0.5 hr. LC-MS showed compound 44-5 was consumed completely and desired mass was detected. The reaction mixture was quenched with aq. NH₄Cl (sat.40.0 mL×3) and extracted with DCM (30.0 mL×3). The combined organic fractions were washed with brine (40.0 mL), dried over Na₂SO₄, filtered and concentrated under reduced pressure to give a residue. The residue was purified by prep-HPLC (column: Phenomenex Synergi C18 150*25 mm*10 um; mobile phase: [water (0.225% FA)−ACN]; B %: 13%-43%, 10 min). Compound 44-7 (13.0 mg, 17.4 umol, 3.94% yield, 83.1% purity) was obtained.

LC-MS: RT=0.809 min, 83.1% purity, (M+H)⁺=620.3
SFC: RT=1.994 min, 2.561 min

### Step 5: Synthesis of Compound 44

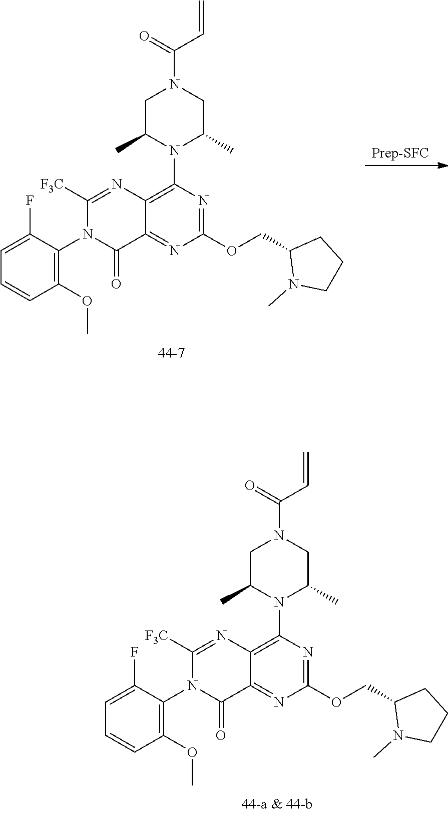 [SMILES: C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1 ; "Prep-SFC" ; C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1]

Compound 44-7 (13.0 mg) was purified by prep-SFC (column: DAICEL CHIRALPAK IC (250 mm*30 mm, 10 um); mobile phase: [0.1% NH₃H₂O MEOH]; B %: 50%-50%, 2.2; 60 min). 44-a (5.00 mg, 7.27 umol, 41.7% yield, 90.1% purity) and 44-b (4.00 mg, 5.98 umol, 34.32% yield, 92.7% purity) were obtained.

44-a:

LC-MS: RT=1.988 min, (M+H)⁺=620.3

HPLC: 80.0% purity

¹H NMR (400 MHz, CDCl₃): δ 7.52-7.46 (m, 1H), 6.91-6.84 (m, 2H), 6.62-6.55 (m, 1H), 6.47-6.43 (m, 1H), 5.81 (d, J=11.6 Hz, 1H), 4.77-4.50 (m, 2H), 4.20-4.04 (m, 2H), 3.80 (s, 3H), 3.75-3.70 (m, 2H), 3.49 (s, 3H), 3.24 (s, 1H), 2.76 (s, 2H), 2.29-2.15 (m, 6H), 1.49-1.47 (m, 6H)

SFC: RT=1.824 min, 99.2% ee

44-b:

LC-MS: RT=1.975 min, (M+H)⁺=620.3

HPLC: 82.5% purity

¹H NMR (400 MHz, CDCl₃): δ 7.44-7.38 (m, 1H), 6.82-6.77 (m, 2H), 6.55-6.48 (m, 1H), 6.40-6.36 (m, 1H), 5.73 (d, J=10.4 Hz, 1H), 4.55-4.39 (m, 2H), 4.13-3.99 (m, 2H), 3.74 (s, 3H), 3.68-3.62 (m, 2H), 3.42 (s, 3H), 3.31-3.20 (m, 1H), 2.57 (s, 2H), 2.20-2.08 (m, 6H), 1.42-1.33 (m, 6H).

SFC: RT=2.325 min, 99.0% ee

### Example 44

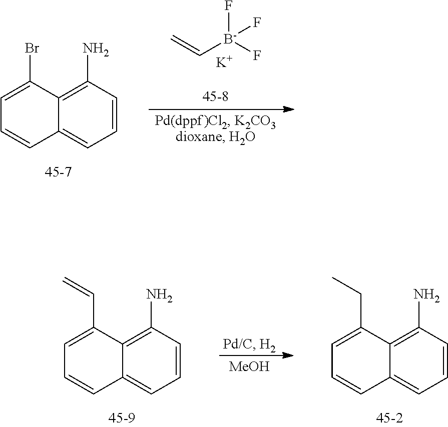

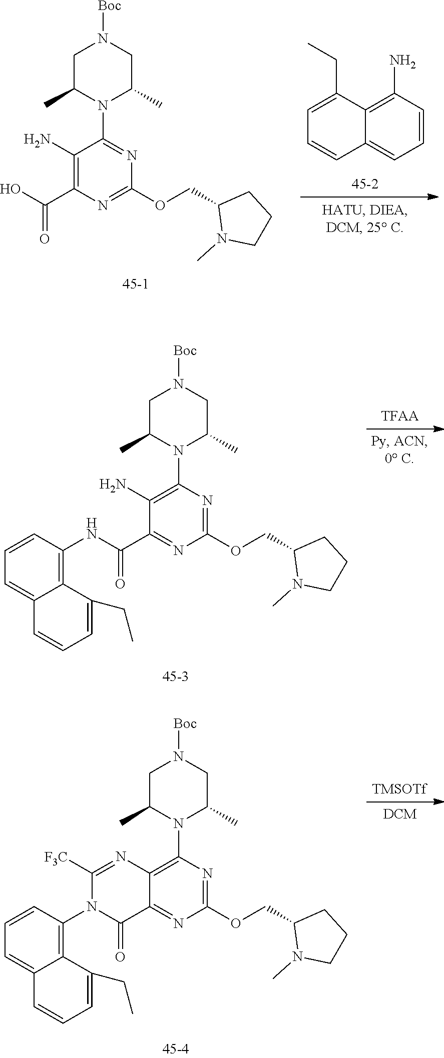 [SMILES: C[C@H]1CN(C(=O)OC(C)(C)C)C[C@H](C)N1c1nc(OC[C@@H]2CCCN2C)nc(C(=O)O)c1N ; CCc1cccc2cccc(N)c12 ; CCc1cccc2cccc(NC(=O)c3nc(OC[C@@H]4CCCN4C)nc(N4[C@@H](C)CN(C(=O)OC(C)(C)C)C[C@@H]4C)c3N)c12 ; "TMSOTf" ; "TFAA" ; CCc1cccc2cccc(-n3c(C(F)(F)F)nc4c(N5[C@@H](C)CN(C(=O)OC(C)(C)C)C[C@@H]5C)nc(OC[C@@H]5CCCN5C)nc4c3=O)c12]

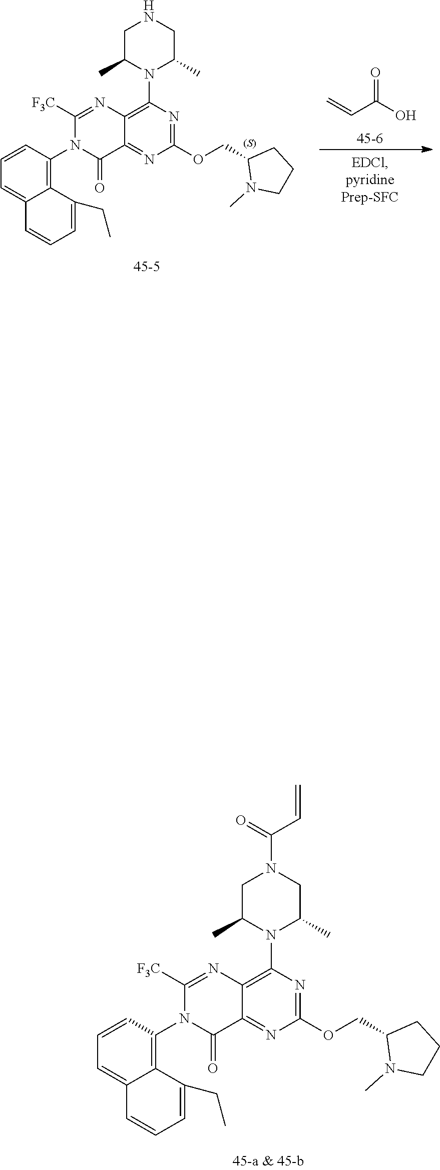

### Step 1: Synthesis of Compound 45-9

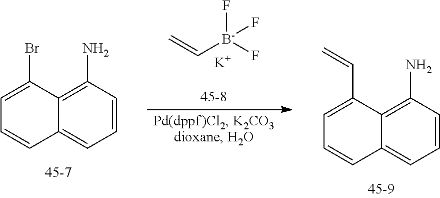

To a solution of compound 45-7 (1.50 g, 6.75 mmol, 1.00 eq) in dioxane (15.0 mL) and H₂O (3.00 mL) was added compound 45-8 (995 mg, 7.43 mmol, 1.10 eq), K₂CO₃ (4.67 g, 33.8 mmol, 5.00 eq) and Pd(dppf)Cl₂ (198 mg, 270 umol, 0.04 eq), then the mixture was stirred at 80° C. for 2 hrs under N₂ atmosphere. LC-MS showed compound 45-7 was consumed completely and one main peak with desired mass was detected. The mixture was filtered and concentrated to give the product. The residue was purified by flash silica gel chromatography (SiO₂, PE/EtOAc=5/1). TLC (PE/EtOAc=5/1, R_{f}=0.4). Compound 45-9 (800 mg, 4.73 mmol, 70.0% yield) was obtained and confirmed by H NMR.

H NMR: (400 MHz, CDCl₃): δ 7.82-7.69 (m, 2H), 7.37-7.33 (m, 1H), 7.30-7.29 (m, 1H), 7.26-7.23 (m, 2H), 6.71 (dd, J₁=2.0 Hz, J₂=6.8 Hz, 1H), 5.60 (dd, J₁=1.6 Hz, J₂=17.2 Hz, 1H), 5.45 (dd, J₁=2.0 Hz, J₂=10.8 Hz, 1H), 4.80-4.12 (m, 2H).

### Step 2. Synthesis of Compound 45-2

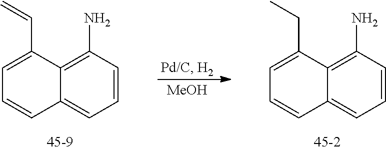

To a solution of compound 45-9 (800 mg, 4.73 mmol, 1.00 eq) in MeOH (10.0 mL) was added Pd/C (80.0 mg, 9.46 mmol, 10.0% purity, 2.00 eq) under N₂ atmosphere, then the mixture was stirred at 25° C. for 4 h under H₂ (15 psi) atmosphere. LC-MS showed compound 45-9 was consumed completely. Several new peaks were shown on LC-MS and ˜57% of desired compound was detected. The mixture was filtered and concentrated to give the product. The residue was purified by HPLC (column: Welch Ultimate XB-CN 250*70*10 um; mobile phase: [Heptane—EtOH (0.10% NH₃H₂O)]; B %: 1%-35%, 15 min). Compound 45-2 (620 mg, 3.62 mmol, 76.6% yield) was obtained and confirmed by H NMR.

H NMR (400 MHz, DMSO-d₆): δ 7.56 (dd, J₁=1.2 Hz, J₂=8.0 Hz, 1H), 7.26-7.23 (m, 1H), 7.18-7.11 (m, 3H), 6.79 (dd, J₁=2.0 Hz, J₂=6.8 Hz, 1H), 5.21 (s, 2H), 3.30-3.24 (m, 2H), 1.28 (t, J=7.6 Hz, 3H).

### Step 3. Synthesis of Compound 45-3

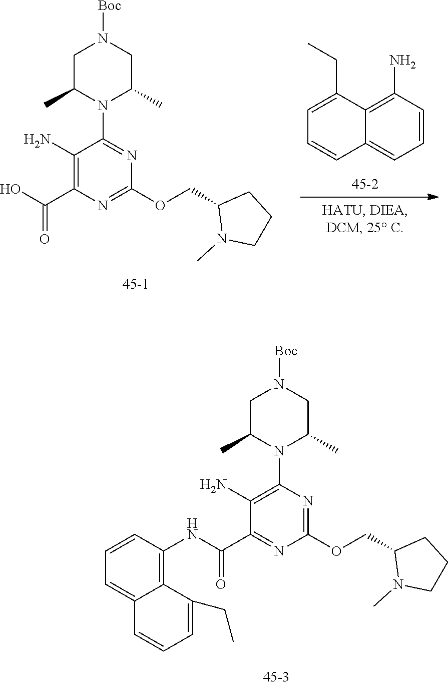 [SMILES: C[C@H]1CN(C(=O)OC(C)(C)C)C[C@H](C)N1c1nc(OC[C@@H]2CCCN2C)nc(C(=O)O)c1N ; CCc1cccc2cccc(N)c12 ; CCc1cccc2cccc(NC(=O)c3nc(OC[C@@H]4CCCN4C)nc(N4[C@@H](C)CN(C(=O)OC(C)(C)C)C[C@@H]4C)c3N)c12]

To a solution of compound 45-1 (500 mg, 1.08 mmol, 1.00 eq) in DCM (10.0 mL) was added DIEA (604 mg, 4.67 mmol, 814 uL, 4.00 eq) and HATU (888 mg, 2.34 mmol, 2.00 eq), the mixture was stirred at 25° C. for 0.5 h, then compound 45-2 (400 mg, 2.34 mmol, 2.00 eq) was added, then the mixture was stirred at 25° C. for 1 hr. LC-MS (EW29126-73-P1A1, product: RT=1.321 min) showed˜29% of compound 1 remained. Several new peaks were shown on LC-MS and ˜31% of desired compound was detected. The reaction mixture was quenched by water (50.0 mL) and then diluted with DCM (50.0 mL) and extracted with DCM (50.0 mL×2). The combined organic layers were dried over Na₂SO₄, filtered and concentrated under reduced pressure to give a residue. The residue was purified by prep-HPLC (column: Welch Ultimate XB-SiOH 250*70*10 um; mobile phase: [Hexane-EtOH (0.1% NH₃—H₂O)]; B %: 1%-20%, 20 min). Compound 45-3 (340 mg, 550 umol, 47.1% yield) was obtained and confirmed by H NMR and SFC.

¹H NMR (400 MHz, CDCl₃): δ 10.28 (s, 1H), 7.81-7.74 (m, 3H), 7.50 (t, J=7.6 Hz, 1H), 7.42-7.34 (m, 2H), 5.74 (s, 2H), 4.35 (dd, J₁=5.2 Hz, J₂=10.4 Hz, 1H), 4.21 (dd, J₁=6.4 Hz, J₂=10.4 Hz, 1H), 4.17-4.08 (m, 1H), 3.90 (s, 2H), 3.42-3.32 (m, 2H), 3.11 (br t, J=8.0 Hz, 1H), 2.75-2.62 (m, 1H), 2.49 (s, 3H), 2.31 (br d, J=8.0 Hz, 1H), 2.12-2.08 (m, 1H), 1.91-1.73 (m, 3H), 1.66 (br s, 6H), 1.50 (s, 9H), 1.05 (d, J=6.4 Hz, 6H).

SFC: RT=1.382 min, 100% ee

### Step 4. Synthesis of Compound 45-4

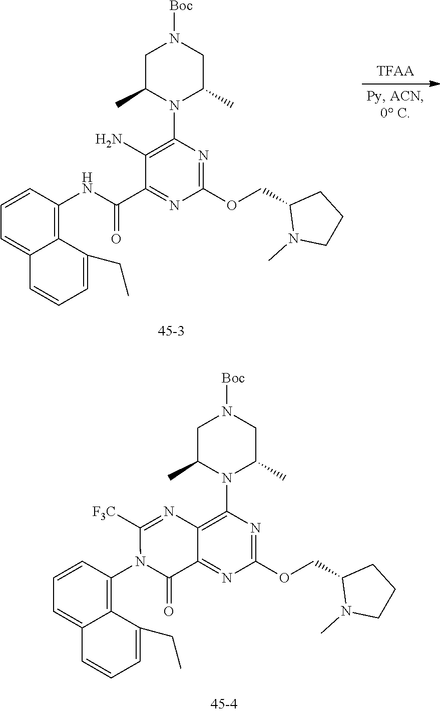 [SMILES: CCc1cccc2cccc(NC(=O)c3nc(OC[C@@H]4CCCN4C)nc(N4[C@@H](C)CN(C(=O)OC(C)(C)C)C[C@@H]4C)c3N)c12 ; "TFAA" ; CCc1cccc2cccc(-n3c(C(F)(F)F)nc4c(N5[C@@H](C)CN(C(=O)OC(C)(C)C)C[C@@H]5C)nc(OC[C@@H]5CCCN5C)nc4c3=O)c12]

To a solution of compound 45-3 (220 mg, 356 umol, 1.00 eq) in MeCN (15.0 mL) was added Py (282 mg, 3.56 mmol, 287 uL, 10.0 eq) and TFAA (449 mg, 2.14 mmol, 297 uL, 6.00 eq) at 0° C. Then the mixture was stirred at 0° C. for 15 min. LC-MS showed compound 45-3 was consumed completely. Several new peaks were shown on LC-MS and ˜54% of desired compound was detected. The reaction mixture was quenched by addition saturated NH₄Cl (100 mL) aqueous solution at 0° C., and then diluted with EtOAc (100 mL) and extracted with solvent EtOAc (100 mL×3). The combined organic layers were dried over anhydrous Na₂SO₄, filtered and concentrated under reduced pressure to give a residue. The crude product was used into the next step without further purification. Compound 45-4 (240 mg, 345 umol, 96.9% yield) was obtained.

LC-MS: RT=0.926 min, (M+H)⁺=696.3

### Step 5. Synthesis of Compound 45-5

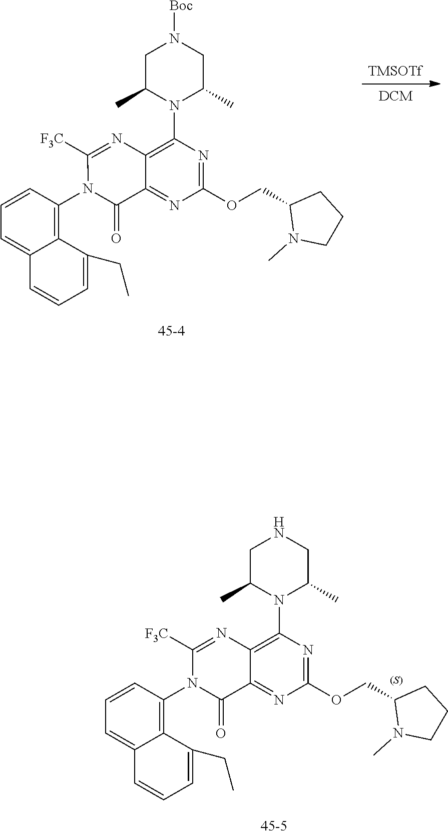

To a solution of compound 45-4 (240 mg, 345 umol, 1.00 eq) in DCM (10.0 mL) was added TMSOTf (115 mg, 517 umol, 93.5 uL, 1.50 eq) at 0° C. Then the mixture was stirred at 0° C. for 15 min. LC-MS showed compound 45-4 was consumed completely. Several new peaks were shown on LC-MS and ˜10% of desired compound was detected. The reaction mixture was quenched by saturated NaHCO₃ aqueous solution (100 mL), and then diluted with DCM (100 mL) and extracted with DCM (100 mL×2). The combined organic layers were washed with brine (50.0 mL), dried over Na₂SO₄, filtered and concentrated under reduced pressure to give a residue. The crude product was used into the next step without further purification. Compound 45-5 (200 mg, 336 umol, 97.3% yield) was obtained.

LC-MS: RT=0.770 min, (M+H)⁺=596.4

### Step 6. Synthesis of Compounds 45-a and 45-b

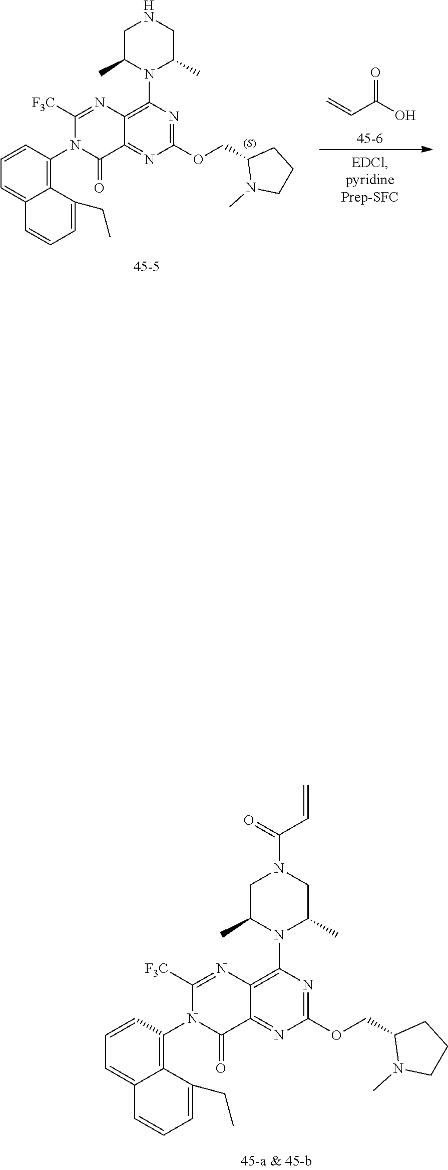

To a solution of compound 45-5 (200 mg, 336 umol, 1.00 eq) in pyridine (5.00 mL) was added compound 45-6 (48.4 mg, 672 umol, 46.1 uL, 2.00 eq) and EDCI (257 mg, 1.34 mmol, 4.00 eq) at 0° C. Then the mixture was stirred at 0° C. for 15 min. LC-MS showed˜9% of compound 45-5 remained. Several new peaks were shown on LC-MS and ˜33% of desired compound was detected. The reaction mixture was quenched by addition saturated NH₄Cl (100 mL) aqueous solution at 0° C., and then diluted with DCM (100 mL) and extracted with solvent DCM (100 mL×3). The combined organic layers were dried over anhydrous Na₂SO₄, filtered and concentrated under reduced pressure to give a residue. The residue was purified by prep-HPLC (column: Welch Ultimate C18 150*25 mm*5 um; mobile phase: [Hexane-EtOH (0.1% NH₃—H₂O)]; B %: 20%-60%, 15 min) to give desired compound, which was further separated by SFC (column: Daicel ChiralPak IG (250*30 mm, 10 um); mobile phase: [0.1% NH₃H₂O ETOH]; B %: 50%-50%, 4.9; 50 min). Compound 45-a (15.0 mg, 22.5 umol, 6.68% yield, 97.2% purity) and Compound 45-b (11.0 mg, 15.9 umol, 4.75% yield, 94.1% purity) were obtained.

45-a:

LC-MS: RT=0.864 min, (M+H)⁺=650.5

HPLC: 97.2% purity

¹H NMR (400 MHz, CDCl₃): δ 8.05 (d, J=8.0 Hz, 1H), 7.84 (d, J=8.0 Hz 1H), 7.56-7.48 (m, 2H), 7.44-7.43 (m, 1H), 7.32 (br d, J=6.8 Hz, 1H), 6.63-6.45 (m, 2H), 5.83 (br d, J=11.2 Hz, 1H), 4.74-4.72 (m, 1H), 4.51 (s, 1H), 4.22 (d, J=14.0 Hz 1H), 4.13-4.09 (m, 1H), 3.85-3.75 (m, 2H), 3.41-3.05 (m, 1H), 2.74-2.68 (m, 5H), 2.17 (s, 1H), 2.01-1.92 (m, 2H), 1.71-1.62 (m, 5H), 1.54-1.46 (m, 6H), 1.26-1.20 (m, 3H).

SFC: RT=1.172 min, 99.9% ee

45-b:

LC-MS: RT=0.857, (M+H)⁺=650.5

HPLC: 94.1% purity

¹H NMR (400 MHz, CDCl₃): δ 8.05 (d, J=8.0 Hz, 1H), 7.84 (d, J=8.0 Hz, 1H), 7.60-7.46 (m, 2H), 7.43-7.41 (m, 1H), 7.30 (br d, J=6.8 Hz, 1H), 6.65-6.56 (m, 1H), 6.49-6.44 (m, 1H), 5.82 (dd, J₁=1.2 Hz, J₂=10 Hz, 1H), 4.78-4.64 (m, 2H), 4.23-4.08 (m, 2H), 3.86-3.74 (m, 2H), 3.55 (br s, 1H), 3.34 (br s, 1H), 2.83 (br s, 2H), 2.74-2.54 (m, 3H), 2.27-2.20 (m, 1H), 2.12-1.91 (m, 6H), 1.51-1.46 (m, 6H), 1.18 (t, J=7.2 Hz, 3H).

SFC: RT=1.750 min, 99.0% ee

### Example 45

### Biochemical Assays

Assay 1: KRAS G12C Nucleotide Exchange Assay

Materials and reagents:

HEPES (Sigma, Cat. No. H3375-500 g)

DMSO (Sigma, Cat. No. 34869-4L)

MgCl₂ (Sigma, Cat. No. M2670-500 g)

GTP (Sigma, Cat. No. G8877)

GDP (Sigma, Cat. No. G7127)

MANT-GTP (SIGMA, 69244-1.5 UMOL)

Glycerol (Sigma, Cat. No. G6279-1 L)

Tween-20 (Sigma, Cat. No. P2287-100 mL)

SOS1 Protein, aa564-1049, 6×His tag (CYTOSKELETON, CS-GE02-XL)

EDTA, pH 8.0 (Gibco, 15575-038, 100 mL)

Pierce Coomassie (Bradford) Protein Assay Kit (Thermo Pierce, 23200)

Illustra NAP-5 Columns (GE, 17085301)

384-well plate (Corning, Product Number 3573)

KRas(1-169) G12C protein

SOS1(594-1049) protein

SOS1(564-1049) protein

KRAS G12C and SOS1 proteins were packed in 5 UL/tube or 20 UL/tube, and frozen in −80° C. refrigerator.

Experiment Method:

### 1. Buffer Preparation:

1×Loading buffer: 20 mM HEPES, pH 7.5, 50 mM NaCl, 0.5 mM MgCl₂, 1 mM DTT, 5 mM EDTA

1×Equilibration buffer: 20 mM HEPES, pH 7.5, 150 mM NaCl, 1 mM MgCl₂, 1 mM DTT

1×Assay buffer: 20 mM HEPES, pH 7.5, 150 mM NaCl, 1 mM MgCl₂, 0.01% Tween-20, 1 mM DTT

### 2. Load Mant GDP to KRAS G12C:

a. A mixed solution of 100 UL mant GDP and KRAS G12C was prepared with 1×loading buffer: 60 um KRAS G12C, 600 um mant GTP, incubated at room temperature for 60 minutes, and the reaction was carried out in dark conditions.

b. Add 1 uL 1 m MgCl₂ (final concentration 10 mm) to stop the reaction, mix the solution upside down in the centrifuge tube, centrifugate for 3-5 seconds, and incubate at room temperature for 30 minutes.

c. At the same time of incubation for 30 minutes, balance nap-5 column with 10 ml ix equipment buffer until no liquid drops.

d. Drop the mixed solution of 100 uL mant GDP and KRAS G12C into the center of nap-5 column. After the sample completely enters nap-5 column, add 400 UL 1× equipment buffer until no liquid drops.

e. Add 500 ul 1× equipment buffer for elution and collect eluant.

f. Determination of KRAS G12C mant GDP with Bradford protein quantitative Kit.

### 3. Nucleotide Exchange Experiment:

a. Transfer 50 NL DMSO/compound to 384 well plate with echo550.

b. 10 uL enzyme mix was added into 384 pore plate and incubated with DMSO/compound for 15 min.

c. Initial reaction with 10 UL Sos1/GTP mix.

d. Immediately after reaction, read ex360/em440 fluorescence value in kinetic mode with Nivo.

### 4. Data Analysis:

a. Use Graphpad software to process data and draw pictures.

b. K value is obtained in Graphpad software by fitting One phase experimental deck model.

c. Z′=1-3*(SdKmax+SdKmin)/(AveKmax−AveKmin)

d. In % is calculated by the following formula:

Inh%=(Kmax−Ksample)/(Kmax−Kmin)*100

Max: KRAS−mGDP+SOS1+GTP

Min: KRAS−mGDP+buffer

The results for exemplary compounds of Formula (I) are shown in Table 1. For the other Example compounds for which the results are not shown, all have an IC₅₀ against KRAS G12C of no more than 60 μM. Some of these compounds have an IC₅₀ against KRAS G12C of no more than 50 μM, some no more than 40 μM, some no more than 30 μM, some no more than 20 μM, or no more than 10 μM, or no more than 5 μM, or no more than 4 μM, or no more than 3 μM, or no more than 2 μM, or no more than 1 μM, no more than 500 nM, no more than 400 nM, no more than 300 nM, no more than 200 nM, or even no more than 100 nM.

TABLE 1

| | KRAS G12C |
|---|---|
| Example No. | IC50 (μM) |
| 1 | 35.4 |
| 2 | 0.69 |
| 3 | 8.237 |
| 4 | 0.56 |
| 5 | 52.33 |
| 6 | 1.78 |
| 7 | 0.39 |
| 8 | 60.72 |
| 9 | 1.07 |
| 10 | 0.84 |
| 11 | 2.94 |
| 12 | 0.90 |
| 13 | 0.39 |
| 14 | 0.86 |
| 15 | 2.93 |
| 16 | 0.82 |
| 17 | 0.48 |
| 18 | 9.97 |
| 19 | 0.37 |
| 20 | 2.88 |
| 21 | 19 |
| 22 | 22.89 |
| 23 | 0.71 |
| 31 | 0.446 |

Assay 2: KRAS GDP FI Assay

1. Prepare compound dilution plate.

2. Transfer Inhibitor/DMSO to assay plate by Echo.

3. Prepare 1× assay buffer.

4. Prepare KRAS G12C mix & SOS1 mix & GTP mix & detection reagent mix.

5. Add KRAS G12C mix, SOS1 mix, GTP mix.

6. Add detection reagent mix to assay plate.

7. Kinetic reading with Ex580/Em620 for 120 min.

The results for exemplary compounds of Formula (I) are shown in Table 2.

TABLE 2

| | KRAS GDP FI |
|---|---|
| Example No. | IC50 (nM) |
| 24 | 132 |
| 25-b | 37 |
| 26-b | 1500 |
| 27-b | 900 |
| 28 | 190 |
| 29-a | 89 |
| 29-b | 127 |
| 30 | 124 |
| 31 | 116 |
| 32-b | 83 |
| 33-b | 127 |
| 36-a | 155 |
| 37 | 178 |
| 38 | 494 |
| 39 | 364 |
| 40-b | 405 |

Assay 3: Tumor Cell Anti-Proliferation Assay (CTG Assay)

Tested tumor cell lines (MIA PaCa-2, NCI-H358, and A549) were seeded to the 96-well plate for overnight, then cells were treated with the test compound at 9 serially diluted concentrations in triplicate. After 3-days incubation with the test compound, the CTG assay was performed to evaluate the IC₅₀. The 3 cell lines were tested in the same manner. Cisplatin were used as the positive control.

Materials and Reagents:

RPMI-1640 (Hyclone, Cat. No.: SH30809.01)

DMEM medium (Hyclone, Cat. No.: SH30243.01)

Ham's F12K (Gbico, Cat. No.: 21127-022)

FBS (Cat. No. 10099-141, Gibco)

CellTiter-Glo® Luminescent Cell Viability Assay (Cat. No. G7572, Promega. Stored at −20° C.).

96-Well Plate, With Lid, White, Flat Bottom, TC-Treated, Polystyrene (Cat. No.: 3610, Corning®)

0.25% Trypsin-EDTA (Cat. No. 25200072, Gibco)

Equipment:

BMRP004; CO2 Incubator, SANYO Electric Co., Ltd (02100400059).

Reverse microscope, Chongguang XDS-1B, Chongqing Guangdian Corp. (TAMIC0200)

Envision 2104 Multi Label Reader, PerkinElmer, USA (TAREA0011)

Vi-Cell XR, Beckman Coulter (TACEL0030)

Method:

### Day −1: Cell Plating for Cell Lines

1. Adjust the cell concentration to the appropriated number with the medium, and for each well of a 96-well plate, add 90 μl cell suspensions (Cell concentration will be adjusted according to the data base or density optimization assay). 2. Incubate the plates for overnight in humidified incubator at 370° C. with 5% CO₂.

### Day 0: T0 Plate Reading and Compound Treatment

3. Add 10 μL culture medium to each well of plate A for T0 reading.

4. Equilibrate the plate and its content at RT for approximately 30 min.

5. Add 50 μL CellTiter-Glo® Reagent to each well for T0 reading.

6. Mix contents for 2 minutes on an orbital shaker to facilitate cell lysis.

7. Allow the plate to incubate at room temperature for 10 minutes to stabilize the luminescent signal. Note: Uneven luminescent signal within standard plates can be caused by temperature gradients, uneven seeding of cells or edge effects in multiwall plates.

8. Place a black BackSeal sticker to the bottom of each plate.

9. Record luminescence using an Envision Multi Label Reader.

10. Dilute the test compound and the positive control (Cisplatin). Add 10 μL of 10× test compound working solutions into the corresponding wells. Incubate the test plates in the humidified incubator at 37° C. with 5% Co₂.

### Day 3: Plate Reading for 3-Day Assay

11. Monitor under the microscope to make sure that the cells in control wells are healthy.

12. After three days incubation, add 50 μL CellTiter-Glo® Reagent to each well.

13. Mix contents for 2 minutes on an orbital shaker to facilitate cell lysis.

14. Allow the plate to incubate at room temperature for 10 minutes to stabilize the luminescent signal.

15. Note: Uneven luminescent signal within standard plates can be caused by temperature gradients, uneven seeding of cells or edge effects in multiwall plates.

16. Place a black BackSeal sticker to the bottom of each plate.

17. Record luminescence using an Envision Multi Label Reader.

Data Analysis:

The data were displayed graphically using GraphPad Prism 5.0. In order to calculate IC₅₀, a dose-response curve was fitted using nonlinear regression model with a sigmoidal dose response. The formula of the surviving rate is shown below, and the IC₅₀ was automatically produced by GraphPad Prism 5.0.

The surviving rate (0%)=(LumTest compound−LumMedium control)/(LumNone treated-LumMedium control)×100%.

LumNone treated-LumMedium control is set as 100% o and LumMedium control is set as 000 surviving rate. T0 value will be presented as percentage of LumNone treated.

Table 3 provides the results for exemplary compounds of Formula (I).

TABLE 3

| | IC50 (nM) | | |
|---|---|---|---|
| | MIA PaCa-2 | NCI-H358 | A549 |
| 2 | 641.8 | 1343 | >5000 |
| 4 | 503.5 | 476 | >5000 |
| 19 | >5000 | >5000 | >5000 |
| 24 | 480 | | |
| 24-a | | 407.1 | |
| 24-b | | 1457 | |
| 25-a | >10 | | |
| 25-b | 1119 | | |
| 26-a | 4376 | | |
| 26-b | 2889 | | |
| 27-a | 2889 | | |
| 27-b | 2686 | | |
| 28 | 1499 | | |
| 29-a | | 148 | |
| 29-b | 906.3 | 28.59 | >10,000 |
| 30 | 72.74 | | |
| 31 | 123.74 | | >10,000 |
| 32-a | 3717 | | |
| 32-b | 188 | | |
| 33-a | 515.0 | 146.8 | >10,000 |
| 33-b | 148.4 | 62.71 | >10,000 |
| 34-a | 1226 | 201.9 | >10,000 |
| 34-b | 678.6 | 81.97 | >10,000 |
| 35-a | 2648 | 1178 | >10,000 |
| 35-b | 915.8 | 205.6 | >10,000 |
| 41-a | 819.8 | 175.8 | |
| 41-b | 1119 | 280.2 | |
| 42 | 875.2 | 263.5 | |
| 43 | 766.3 | 257.2 | |
| 44-a | 7083 | 1983 | |
| 44-b | 480.8 | 146.6 | |
| 45-a | 6446 | 3318 | |
| 45-b | 492.3 | 483.1 | |
| Cisplatin | 14307 | 24979 | 31143 |

### Example 46

### Pharmacokinetics Study

The purpose of this study is to determine the pharmacokinetics parameters in plasma of compounds in ICR mice following intravenous or oral administration.

Test Article Preparation

The formulations were based on sponsor's recommendation and will be prepared by the Testing Facility.

Vehicles: 60% PEG400+10% Ethanol+30% water (pH 7-8)

Test System

Species and Strain: ICR Mice (Male)

Source: Sino-British SIPPR Lab Animal Ltd, Shanghai

Number of Animals: Ordered: 8; Needed: 6

Study Design

| | | | Dose | Dose | | |
|---|---|---|---|---|---|---|
| | Number of | Dose Level | Conc. | Volume | Route of | |
| Group | animals | (mg/kg) | (mg/mL) | (mL/kg) | Dosing | Collection |
| 1 | 3 | 1 | 0.2 | 5 | IV | Plasma |
| 2 | 3 | 10 | 1 | 10 | PO* | Plasma |

*The animals were fasted prior to oral administration. Food supply to the animals dosed orally were resumed 4 hours post-dose.

Administration

The test article was be administered via a single IV or PO dosing.

Collection Intervals

IV group: Post-dose at 5 min, 15 min, 30 min, 1 h, 2 h, 4 h, 8 h and 24 h.

PO group: Post-dose at 15 min, 30 min, 1 h, 2 h, 4 h, 6 h, 8 h and 24 h.

30˜40 μL/sample. Samples were be placed in tubes containing heparin sodium and stored on ice until centrifuged.

Analysis Procedure

The PK blood samples were centrifuged at approximately 6800G for 6 minutes at 2-8° C. and the resulting plasma was transferred to appropriately labeled tubes within 2 hour of blood collection/centrifugation and stored frozen at approximately −70° C.

Method development and biological samples analysis for the test articles (Sodium heparin anticoagulant) were be performed by Testing Facility by means of LC-MS/MS. The analytical results were be confirmed using quality control samples for intra-assay variation. The accuracy of >66.7% of the quality control samples were be between 80-120% of the known value(s).

Pharmacokinetics Analysis

Standard set of parameters including Area Under the Curve (AUC₍₀₋ₜ₎ and AUC_{(0-∞)}), elimination half-live (T_{1/2}), maximum plasma concentration (Cmax) and time to reach maximum plasma concentration (Tmax) and other parameters were be calculated using Phoenix WinNonlin 7.0 (Pharsight, USA) by the Study Director.

TABLE 4
provides the results for exemplary compounds of Formula (I).

| | | 29-b | 33-b |
|---|---|---|---|
| IV | Dose (mpk) | 1 | 1 |
| | C₀ (nM) | 658.53 | 434.85 |
| | T_{1/2} (h) | 1.59 | 2.74 |
| | Cl (mL/Kg/min) | 25.72 | 19.89 |
| | Vd (L/kg) | 2.97 | 4.13 |
| | AUC (nM · h) | 993.28 | 1,170.65 |
| PO | Dose (mpk) | 10 | 10 |
| | Cmax (nM) | 729.06 | 792.82 |
| | T_{1/2} (h) | 2.39 | 2.76 |
| | Tmax (h) | 0.42 | 1.00 |
| | AUC (nM · h): | 2,583.31 | 6,776.89 |
| | F (%) | 26.74 | 57.89 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What claimed is:

1. A compound having Formula (I): 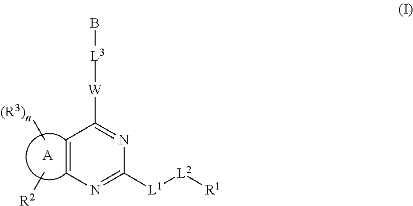
or a pharmaceutically acceptable salt thereof,
wherein
Ring A is selected from the group consisting of saturated or partially unsaturated cycloalkyl, saturated or partially unsaturated heterocyclyl, and heteroaryl;
L¹ is a bond, O, S or N(R^{a});
L² is selected from the group consisting of a bond, alkyl, alkenyl, alkynyl, heteroalkyl, heteroalkenyl, and heteroalkynyl;
R¹ is selected from the group consisting of alkyl, alkenyl, alkynyl, heteroalkyl, heteroalkenyl, heteroalkynyl, saturated or partially unsaturated cycloalkyl, saturated or partially unsaturated heterocyclyl, aryl, and heteroaryl, wherein each of alkyl, alkenyl, alkynyl, cycloalkyl, heterocyclyl, aryl, heteroaryl is optionally substituted with one or more R^{b};
R² is selected from the group consisting of H, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R^{c},
R³ is selected from the group consisting of hydrogen, oxo, halogen, cyano, hydroxyl, —NR^{d}R^{e}, —C(O)NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R; or
R⁴ and R⁵, R⁴ and R⁶, R⁴ and R⁷, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR′R^{d}, carboxy, carbamoyl, aryl or heteroaryl;
W is saturated or partially unsaturated cycloalkyl, or saturated or partially unsaturated heterocyclyl, wherein each of cycloalkyl and heterocyclyl is optionally substituted with one or more R^{g},
L³ is a bond, alkyl or —NR^{d}—;
B is an electrophilic moiety capable of forming a covalent bond with a cysteine residue at position 12 of a K-Ras G12C mutant protein;
R^{a} is independently hydrogen or alkyl;
each R^{b} is independently selected from the group consisting of oxo, cyano, halogen, hydroxy, acyl, —NR^{d}R^{e}, carbamoyl, carboxyl, alkyl, alkenyl, alkynyl, alkoxyl, alkoxylalkyl, cycloalkylalkyl, cycloalkyl, heterocyclyl, aryl, heteroaryl;
each R′ is independently selected from the group consisting of oxo, halogen, cyano, hydroxy, —NR^{d}R^{e}, —C(O)OR^{a}, —C(O)N(R^{d})(R^{e}), alkyl, alkenyl, alkynyl, heteroalkyl, heteroalkenyl, heteroalkynyl, alkoxyl, saturated or partially unsaturated cycloalkyl, saturated or partially unsaturated heterocyclyl, aryl, and heteroaryl;
each of R^{d} and R⁸ is independently selected from the group consisting of hydrogen, alkyl, alkenyl, alkynyl, heteroalkyl, heteroalkenyl, heteroalkynyl, aryl, heteroaryl, wherein each of alkyl, alkenyl, alkynyl, heteroalkyl, heteroalkenyl, heteroalkynyl, aryl and heteroaryl is optionally substituted with cyano, halogen, hydroxy, or amino;
each R^{f} is independently selected from the group consisting of oxo, halogen, cyano, hydroxy, —NR^{c}R^{d}, alkyl, alkenyl, alkynyl, heteroalkyl, heteroalkenyl, heteroalkynyl, cycloalkyl, heterocyclyl, aryl, and heteroaryl;
each R^{g} is independently selected from the group consisting of oxo, cyano, halogen, hydroxy, —NR^{d}R^{e}, carbamoyl, carboxy, alkyl, alkenyl, alkynyl, heteroalkyl, heteroalkenyl, heteroalkynyl, saturated or partially unsaturated cycloalkyl, and saturated or partially unsaturated heterocyclyl, wherein each of alkyl, alkenyl, alkynyl, heteroalkyl, heteroalkenyl, heteroalkynyl, cycloalkyl, and heterocyclyl is optionally substituted with cyano, halogen, hydroxy, —NR^{d}R^{e}, carboxy, carbamoyl, haloalkyl, aryl or heteroaryl;
n is 0, 1, 2, 3 or 4.

2. The compound of claim 1, or a pharmaceutically acceptable salt thereof, wherein Ring A is saturated or partially unsaturated cycloalkyl.

3. The compound of claim 1, or a pharmaceutically acceptable salt thereof, wherein Ring A is saturated or partially unsaturated heterocyclyl.

4. The compound of claim 1, or a pharmaceutically acceptable salt thereof, wherein Ring A is heteroaryl.

5. The compound of claim 1, or a pharmaceutically acceptable salt thereof, wherein L¹ is O.

6. The compound of claim 1 or 5, or a pharmaceutically acceptable salt thereof, wherein L² is a bond.

7. The compound of claim 1 or 5, or a pharmaceutically acceptable salt thereof, wherein L² is alkyl.

8. The compound of claim 7, or a pharmaceutically acceptable salt thereof, wherein L² is methyl, ethyl or propyl.

9. The compound of claim 8, or a pharmaceutically acceptable salt thereof, wherein R¹ is saturated or partially unsaturated cycloalkyl, or saturated or partially unsaturated heterocyclyl, wherein each cycloalkyl and heterocyclyl is optionally substituted with one or more R^{b}.

10. The compound of claim 9, or a pharmaceutically acceptable salt thereof, wherein each R^{b} is selected from the group consisting of oxo, cyano, halogen, hydroxy, acyl, —NR^{d}R^{e}, alkyl, alkoxyl, alkoxylalkyl and cycloalkylalkyl.

11. The compound of claim 9, or a pharmaceutically acceptable salt thereof, wherein R¹ is saturated or partially unsaturated heterocyclyl selected from the group consisting of: 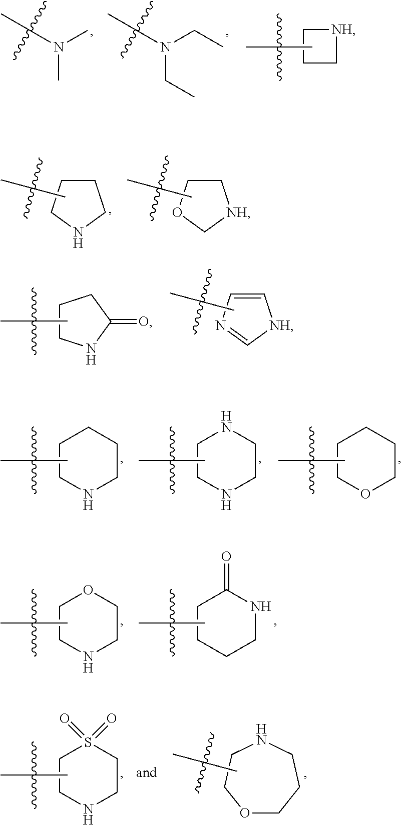
each of which is optionally substituted with one or more R^{b}.

12. The compound of claim 11, or a pharmaceutically acceptable salt thereof, wherein each R^{b} is selected from the group consisting of oxo, halogen, acyl, —NR^{d}R^{e}, alkyl, alkoxyl, alkoxylalkyl, and cycloalkylalkyl.

13. The compound of claim 11, or a pharmaceutically acceptable salt thereof, wherein each R^{b} is halogen or alkyl.

14. The compound of claim 11, or a pharmaceutically acceptable salt thereof, wherein each R^{b} is fluoro, chloro or methyl.

15. The compound of claim 11, or a pharmaceutically acceptable salt thereof, wherein R¹ is

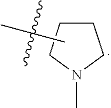

16. The compound of claim 11, or a pharmaceutically acceptable salt thereof, wherein R¹ is

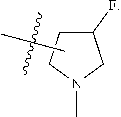

17. The compound of claim 1, or a pharmaceutically acceptable salt thereof, wherein -L¹-L²-R¹ is

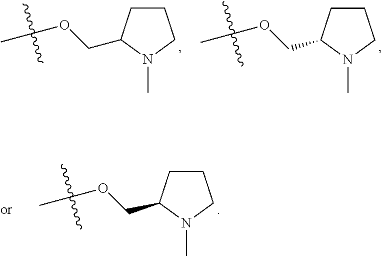

18. The compound of claim 1, or a pharmaceutically acceptable salt thereof, wherein -L¹-L²-R¹ is

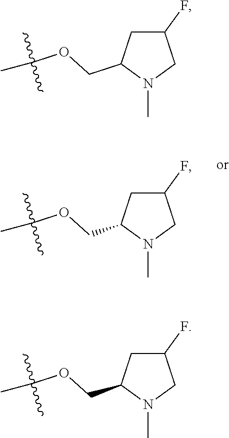

19. The compound of claim 1, or a pharmaceutically acceptable salt thereof, wherein R² is aryl optionally substituted with one or more R^{c}.

20. The compound of claim 19, or a pharmaceutically acceptable salt thereof, wherein each R^{c} is selected from the group consisting of halogen, cyano, hydroxyl, alkyl, alkenyl, alkoxyl, and saturated or partially unsaturated cycloalkyl.

21. The compound of claim 1, or a pharmaceutically acceptable salt thereof, wherein R² is aryl selected from the group consisting of: 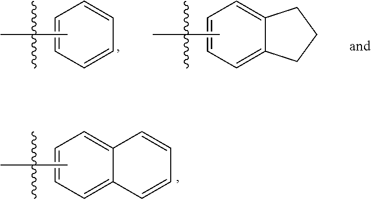
each of which is optionally substituted with one or more R^{c}.

22. The compound of claim 21, or a pharmaceutically acceptable salt thereof, wherein each R^{c} is selected from the group consisting of halogen, hydroxyl, alkyl, alkenyl, alkoxyl, and saturated or partially unsaturated cycloalkyl.

23. The compound of claim 21, or a pharmaceutically acceptable salt thereof, wherein each R^{c} is selected from the group consisting of halogen, hydroxyl, alkyl, alkenyl, alkoxyl, and saturated cycloalkyl.

24. The compound of claim 21, or a pharmaceutically acceptable salt thereof, wherein each R^{c} is selected from the group consisting of fluoro, chloro, hydroxyl, methyl, ethyl, 2-methylpropenyl, methoxyl, and cyclopropyl.

25. The compound of claim 1, or a pharmaceutically acceptable salt thereof, wherein R² is selected from the group consisting of:

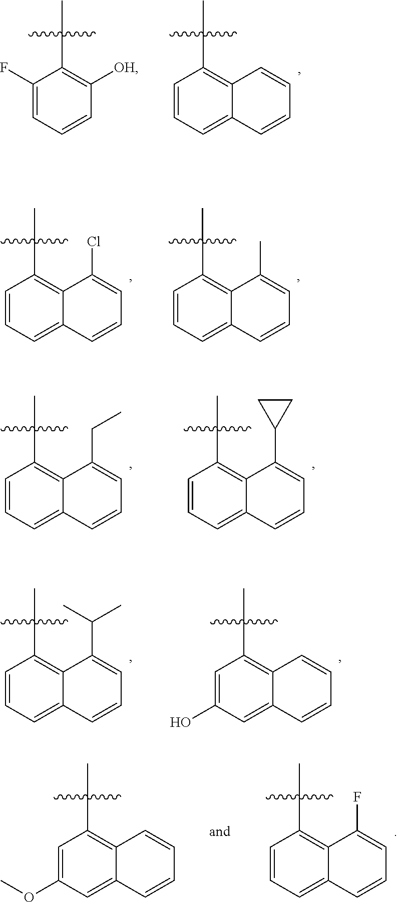

26. The compound of claim 1, or a pharmaceutically acceptable salt thereof, wherein R² is heteroaryl optionally substituted with one or more R^{c}.

27. The compound of claim 26, or a pharmaceutically acceptable salt thereof, wherein each R^{c} is selected from the group consisting of halogen, cyano, hydroxyl, —NR^{d}R^{e}, alkyl, alkenyl, alkoxyl, and saturated or partially unsaturated cycloalkyl.

28. The compound of claim 1, or a pharmaceutically acceptable salt thereof, wherein R² is heteroaryl selected from the group consisting of: 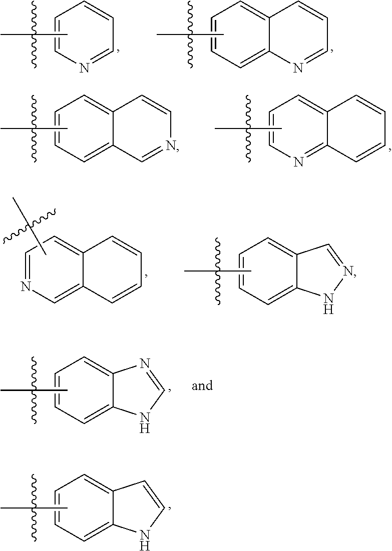
each of which is optionally substituted with one or more R^{c}.

29. The compound of claim 28, or a pharmaceutically acceptable salt thereof, wherein each R^{c} is selected from the group consisting of halogen, cyano, hydroxyl, —NR^{d}R^{e}, alkyl, alkenyl, alkoxyl, and saturated or partially unsaturated cycloalkyl.

30. The compound of claim 29, or a pharmaceutically acceptable salt thereof, wherein each R^{c} is halogen or alkyl.

31. The compound of claim 30, or a pharmaceutically acceptable salt thereof, wherein each R^{c} is selected from the group consisting of fluoro, chloro, methyl, and ethyl.

32. The compound of claim 1, or a pharmaceutically acceptable salt thereof, wherein R² is selected from the group consisting of:

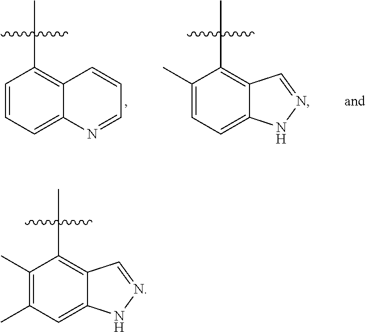

33. The compound of claim 1, or a pharmaceutically acceptable salt thereof, wherein R³ is selected from the group consisting of oxo, alkyl and aryl, wherein alkyl and aryl is optionally substituted with one or more R^{c}.

34. The compound of claim 1, or a pharmaceutically acceptable salt thereof, wherein R^{c} is selected from the group consisting of halogen, cyano, hydroxy, —NR^{c}R^{d}, alkyl.

35. The compound of claim 1, or a pharmaceutically acceptable salt thereof, wherein R³ is selected from the group consisting of oxo, methyl, ethyl, trifluoromethyl and phenyl.

36. The compound of claim 1, or a pharmaceutically acceptable salt thereof, wherein two R³, together with the atoms to which they are each attached, form saturated or partially unsaturated cycloalkyl optionally substituted with one or more substituents selected from the group consisting of cyano, halogen, hydroxy, and —NR^{c}R^{d}

37. The compound of claim 1, or a pharmaceutically acceptable salt thereof, wherein W is saturated or partially unsaturated heterocyclyl optionally substituted with one or more R^{g}.

38. The compound of claim 35, or a pharmaceutically acceptable salt thereof, wherein R^{g} is alkyl optionally substituted with one or more substituents selected from the group consisting of cyano, halogen, and hydroxyl.

39. The compound of claim 1, or a pharmaceutically acceptable salt thereof, wherein W is heterocyclyl selected from the group consisting of: 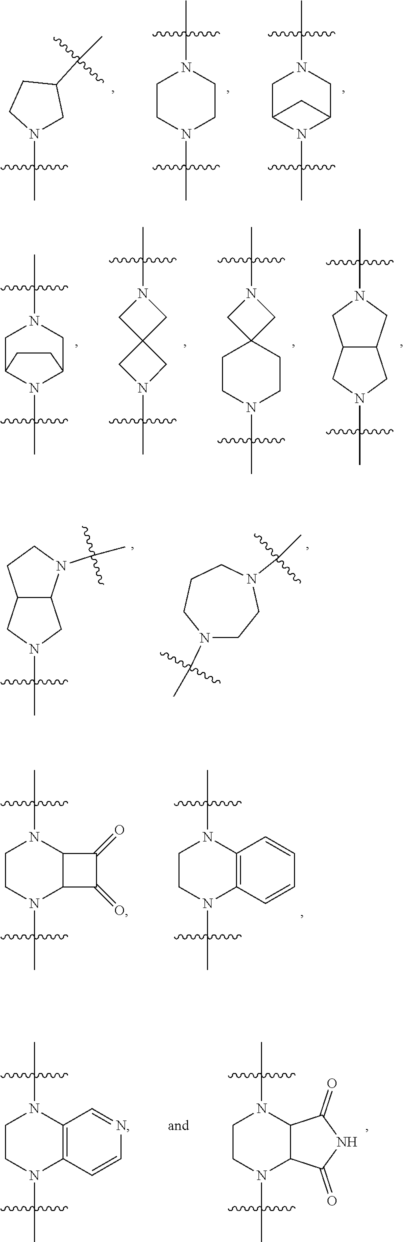
each of which is optionally substituted with one or more R^{g}.

40. The compound of claim 37, or a pharmaceutically acceptable salt thereof, wherein each R^{g} is alkyl optionally substituted with cyano.

41. The compound of claim 38, or a pharmaceutically acceptable salt thereof, wherein each R^{g} is methyl optionally substituted with cyano.

42. The compound of claim 1, or a pharmaceutically acceptable salt thereof, wherein W is selected from the group consisting of:

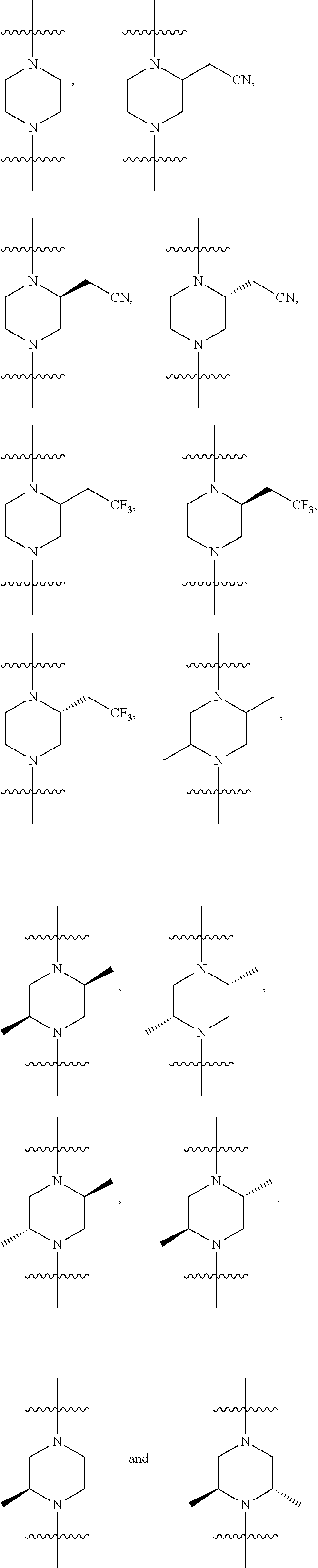

43. The compound of claim 1, or a pharmaceutically acceptable salt thereof, wherein L³ is a bond or —NR^{d}—.

44. The compound of claim 1, or a pharmaceutically acceptable salt thereof, wherein B is selected from the group consisting of:

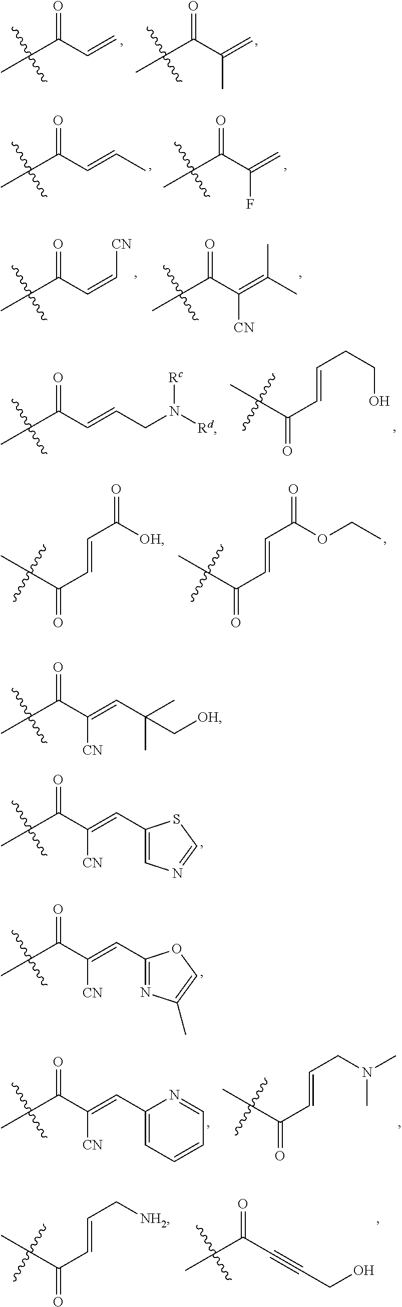

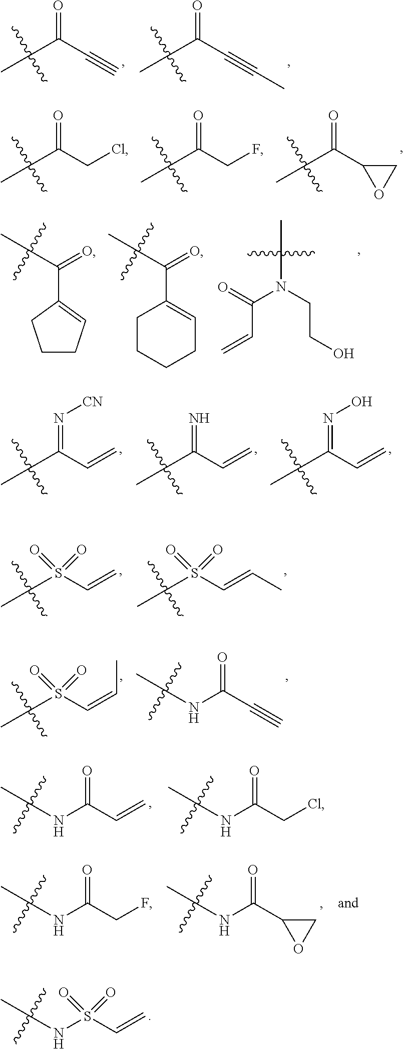

45. The compound of claim 1, or a pharmaceutically acceptable salt thereof, having a formula selected from the group consisting of: 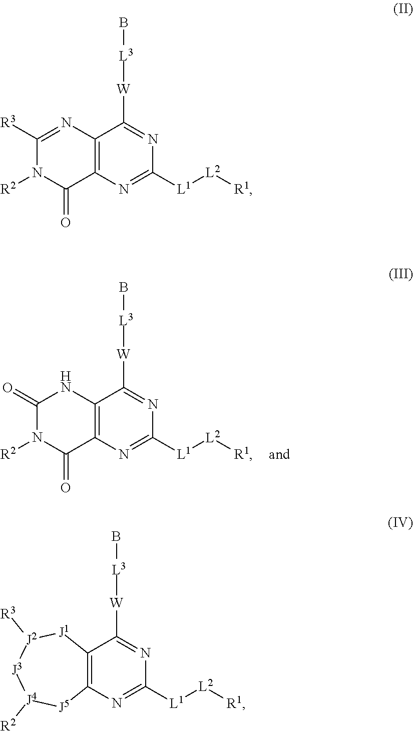
wherein
J¹ is absent, CH(R⁴), NR⁴, SO₂ or P(O)CH₃;
J² is absent, CR⁵, N, SO₂ or P(O)CH₃;
J³ is absent, CH(R⁶), NR⁶, SO₂ or P(O)CH₃;
J⁴ is absent, CR⁷, N, SO₂ or P(O)CH₃;
J⁵ is absent, CH(R⁸), NR⁸, SO₂ or P(O)CH₃;
R⁴, R⁵, R⁶, R⁷ and R⁸ are each independently selected from the group consisting of hydrogen, oxo, halogen, cyano, hydroxyl, —NR^{d}R^{e}, alkyl, alkenyl, alkynyl, heteroalkyl, heteroalkenyl, heteroalkynyl, saturated or partially unsaturated cycloalkyl, saturated or partially unsaturated heterocyclyl, aryl and heteroaryl, wherein each of alkyl, alkenyl, alkynyl, heteroalkyl, heteroalkenyl, heteroalkynyl, cycloalkyl, heterocyclyl, aryl and heteroaryl is optionally substituted with one or more R^{f}; or
R² and any one of R⁴, R⁵, R⁶, R⁷ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or
R³ and any one of R⁴, R⁵, R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or
R⁴ and any one of R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl; or
R⁶ and R⁸, together with the atoms to which they are each attached, form saturated or partially unsaturated cycloalkyl, or saturated or partially unsaturated heterocyclyl, wherein each of cycloalkyl and heterocyclyl is optionally substituted with cyano, halogen, hydroxy, —NR^{c}R^{d}, carboxy, carbamoyl, aryl or heteroaryl.

46. The compound of claim 45, or a pharmaceutically acceptable salt thereof, having a formula selected from the group consisting of:

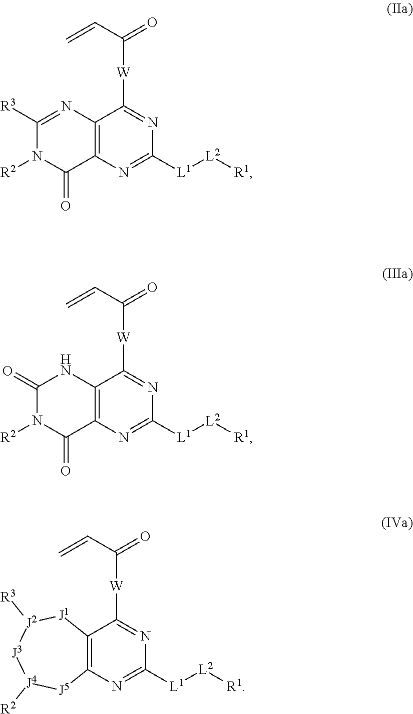

47. The compound of claim 45, or a pharmaceutically acceptable salt thereof, having a formula selected from the group consisting of: 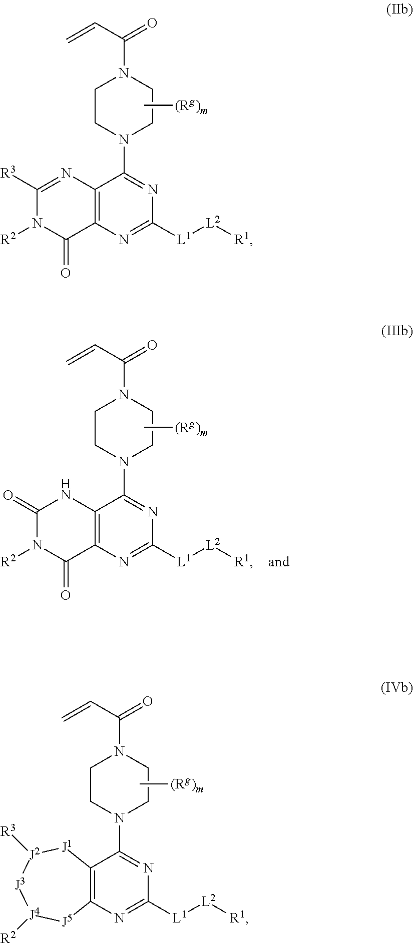
wherein m is 0, 1, 2, 3 or 4.

48. The compound of claim 45, or a pharmaceutically acceptable salt thereof, having a formula selected from the group consisting of: 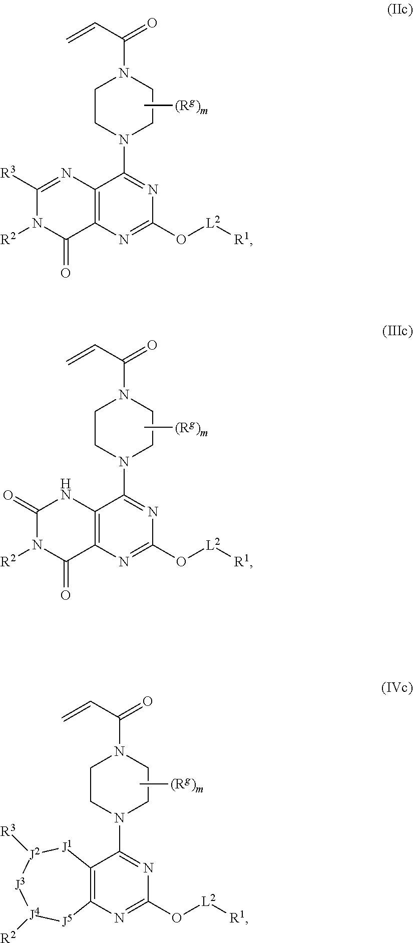
wherein m is 0, 1, 2, 3 or 4.

49. The compound of claim 45, or a pharmaceutically acceptable salt thereof, having a formula selected from the group consisting of:

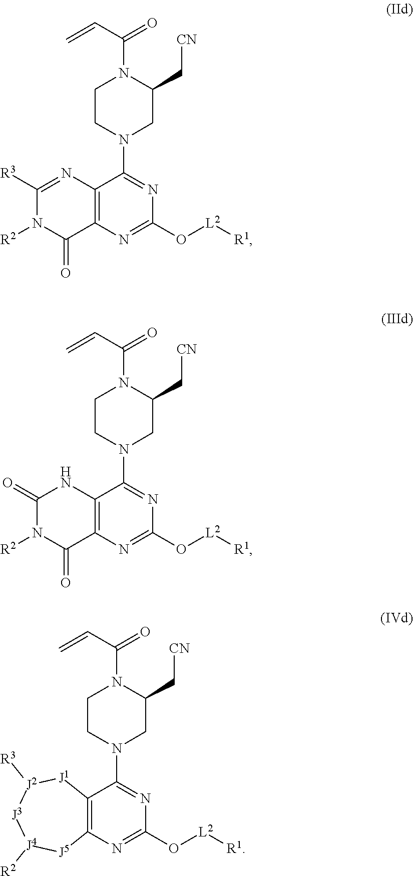

50. The compound of claim 45, or a pharmaceutically acceptable salt thereof, having a formula selected from the group consisting of:

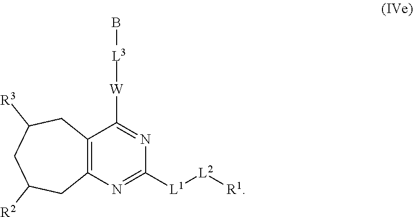

51. The compound of claim 45, or a pharmaceutically acceptable salt thereof, having a formula selected from the group consisting of:

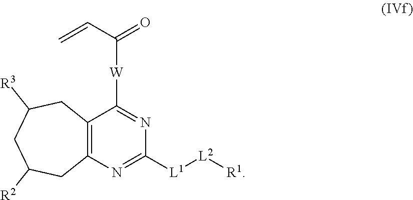

52. The compound of claim 45, or a pharmaceutically acceptable salt thereof, having a formula selected from the group consisting of: 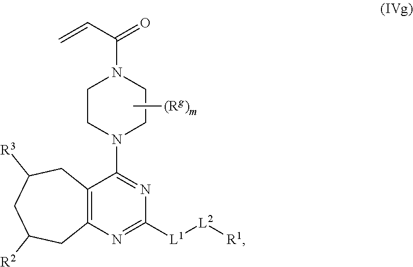
wherein m is 0, 1, 2, 3 or 4.

53. The compound of claim 45, or a pharmaceutically acceptable salt thereof, having a formula selected from the group consisting of: 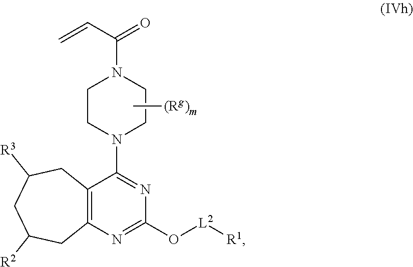
wherein m is 0, 1, 2, 3 or 4.

54. The compound of claim 45, or a pharmaceutically acceptable salt thereof, having a formula selected from the group consisting of:

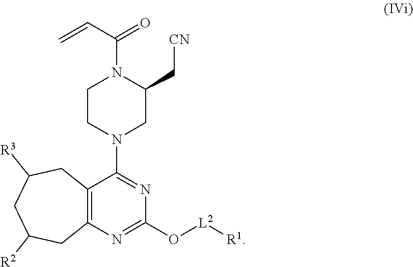

55. The compound of claim 45, or a pharmaceutically acceptable salt thereof, having a formula selected from the group consisting of:

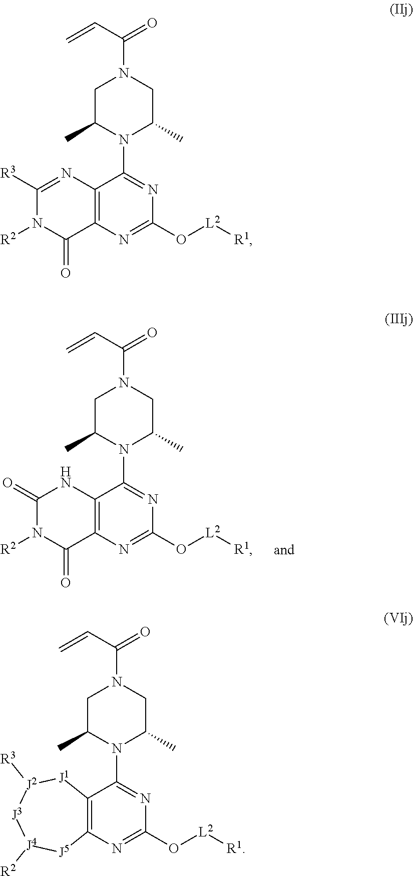

56. The compound of claim 45, or a pharmaceutically acceptable salt thereof, having a formula selected from the group consisting of:

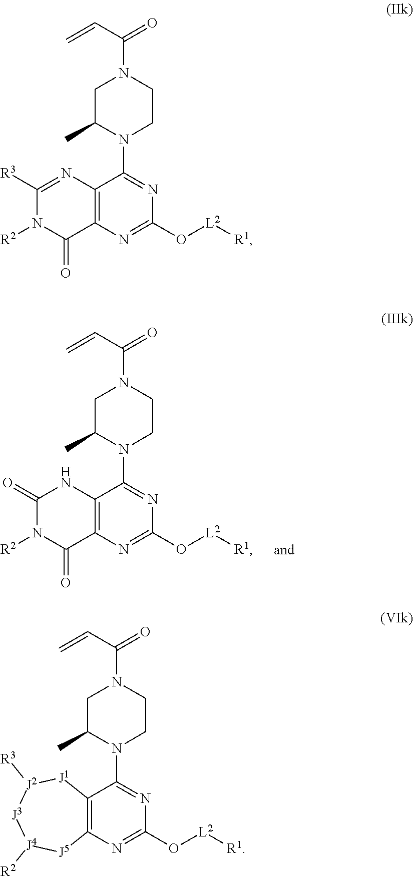

57. The compound of any one of claims 45-56, or a pharmaceutically acceptable salt thereof, wherein L² is alkyl.

58. The compound of any one of claims 45-57, or a pharmaceutically acceptable salt thereof, wherein R¹ is

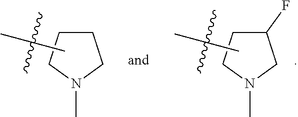

59. The compound of any one of claims 45-58, or a pharmaceutically acceptable salt thereof, wherein R³ is selected from methyl, ethyl or trifluoromethyl.

60. The compound of claim 1, or a pharmaceutically acceptable salt thereof, having a formula selected from the group consisting of:

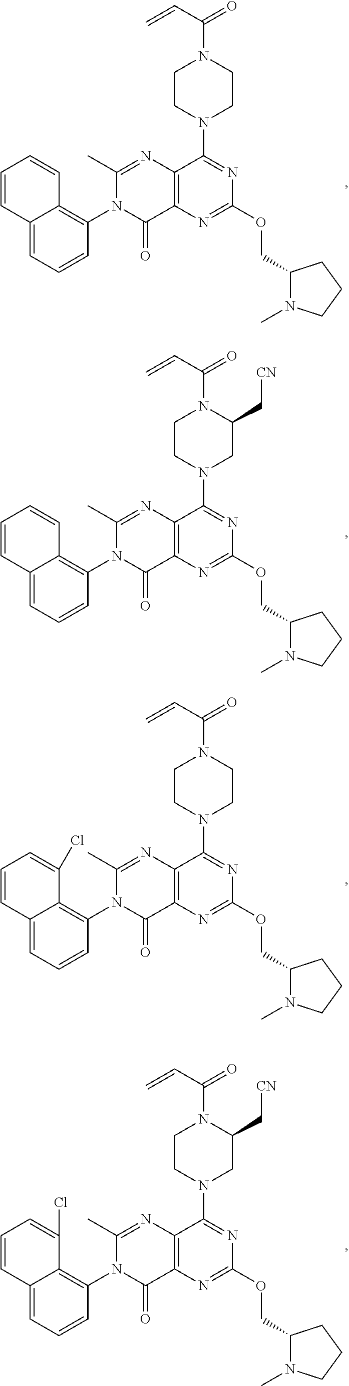

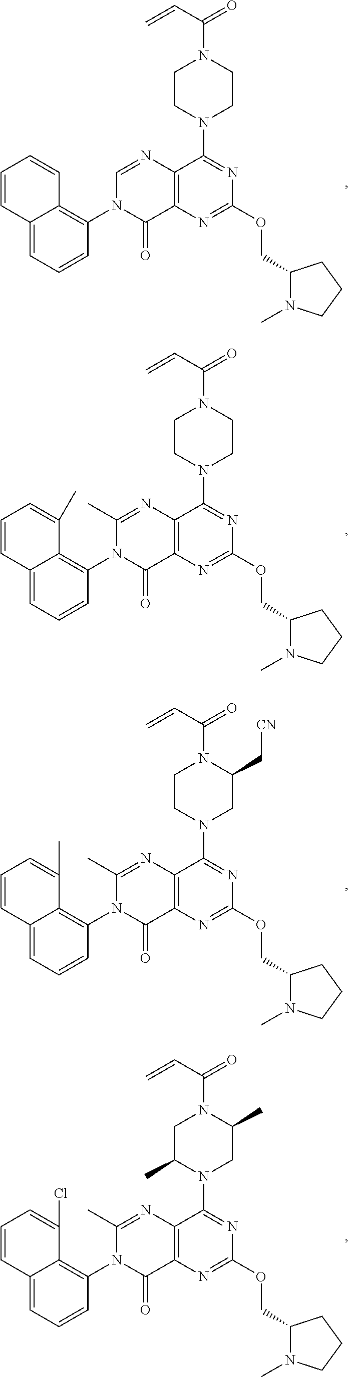

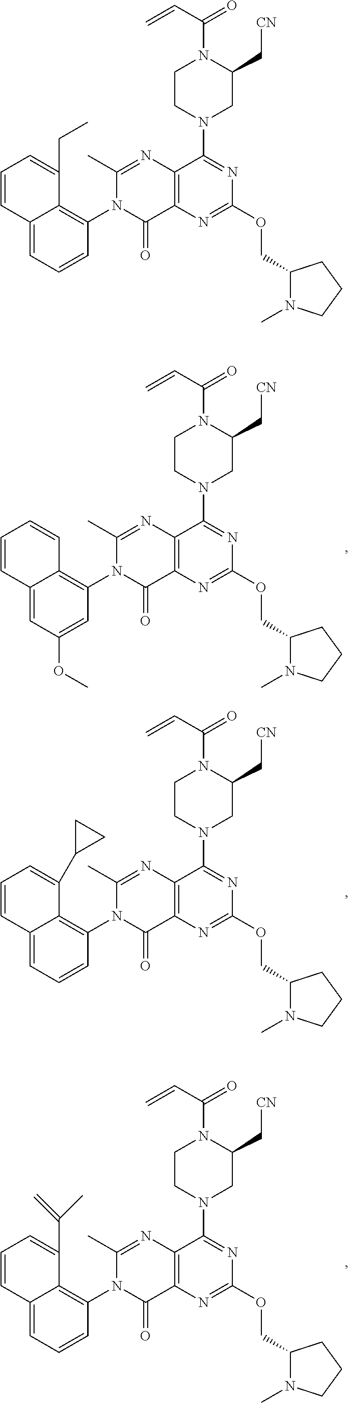

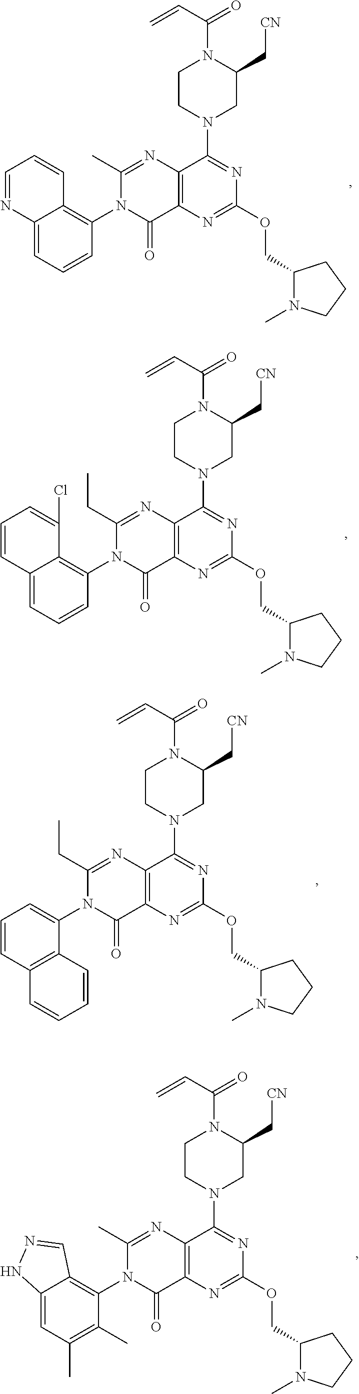 [SMILES: C=CC(=O)N1CCN(c2nc(OC[C@@H]3CCCN3C)nc3c(=O)n(-c4cccc5ncccc45)c(C)nc23)C[C@@H]1CC#N ; C=CC(=O)N1CCN(c2nc(OC[C@@H]3CCCN3C)nc3c(=O)n(-c4cccc5cccc(Cl)c45)c(CC)nc23)C[C@@H]1CC#N ; C=CC(=O)N1CCN(c2nc(OC[C@@H]3CCCN3C)nc3c(=O)n(-c4cccc5ccccc45)c(CC)nc23)C[C@@H]1CC#N ; C=CC(=O)N1CCN(c2nc(OC[C@@H]3CCCN3C)nc3c(=O)n(-c4c(C)c(C)cc5[nH]ncc45)c(C)nc23)C[C@@H]1CC#N]

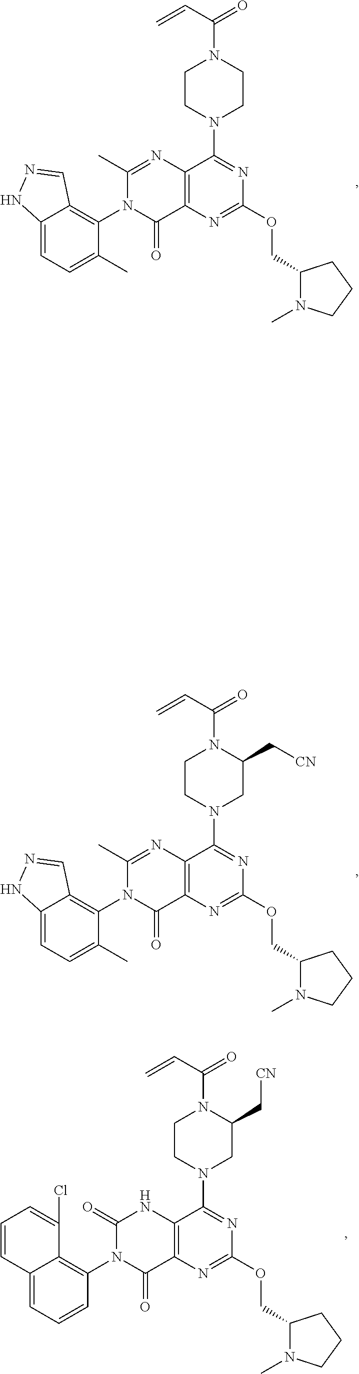 [SMILES: C=CC(=O)N1CCN(c2nc(OC[C@@H]3CCCN3C)nc3c(=O)n(-c4c(C)ccc5[nH]ncc45)c(C)nc23)CC1 ; C=CC(=O)N1CCN(c2nc(OC[C@@H]3CCCN3C)nc3c(=O)n(-c4c(C)ccc5[nH]ncc45)c(C)nc23)C[C@@H]1CC#N ; C=CC(=O)N1CCN(c2nc(OC[C@@H]3CCCN3C)nc3c(=O)n(-c4cccc5cccc(Cl)c45)c(=O)[nH]c23)C[C@@H]1CC#N]

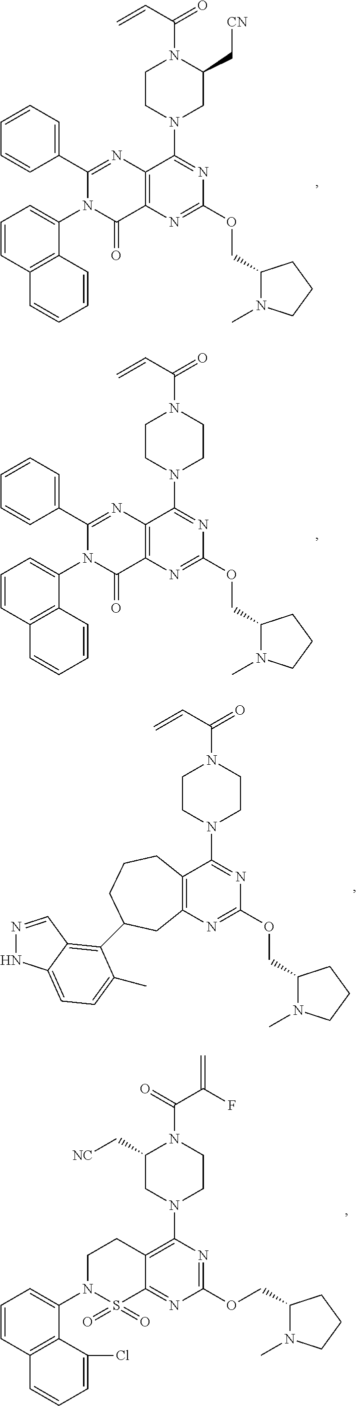

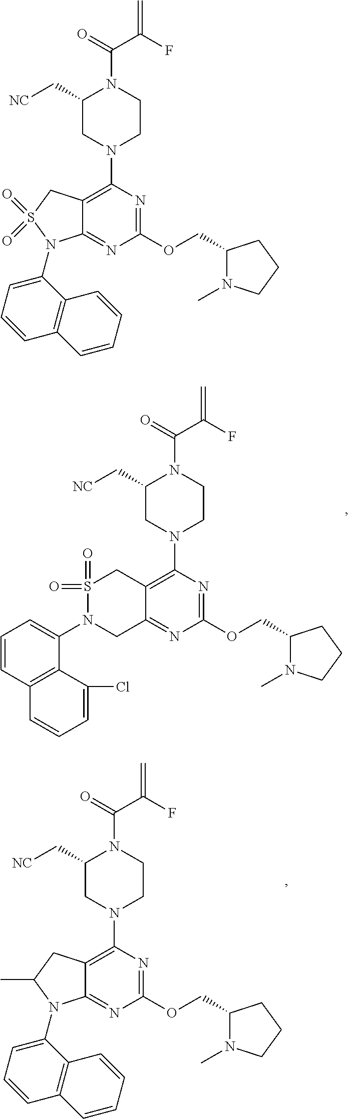 [SMILES: C=C(F)C(=O)N1CCN(c2nc(OC[C@@H]3CCCN3C)nc3c2CS(=O)(=O)N3c2cccc3ccccc23)C[C@@H]1CC#N ; C=C(F)C(=O)N1CCN(c2nc(OC[C@@H]3CCCN3C)nc3c2CS(=O)(=O)N(c2cccc4cccc(Cl)c24)C3)C[C@@H]1CC#N ; C=C(F)C(=O)N1CCN(c2nc(OC[C@@H]3CCCN3C)nc3c2CC(C)N3c2cccc3ccccc23)C[C@@H]1CC#N]

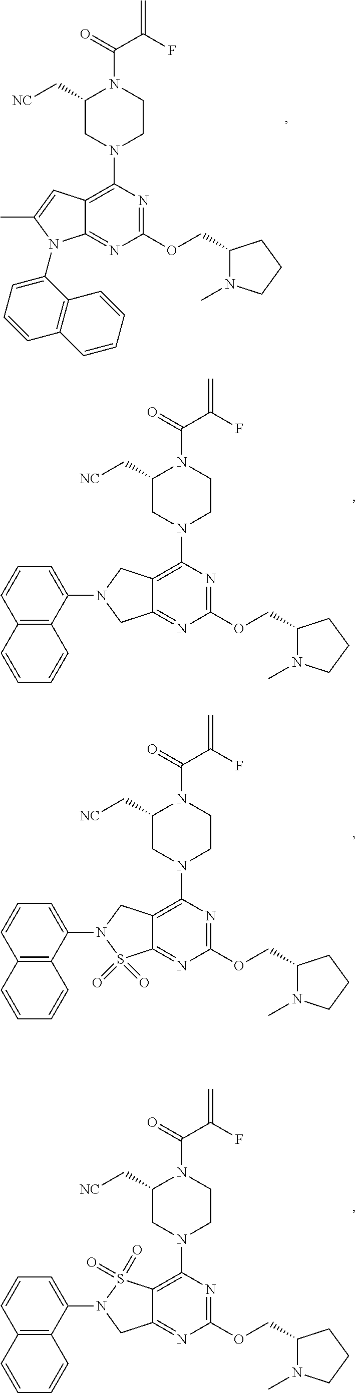 [SMILES: C=C(F)C(=O)N1CCN(c2nc(OC[C@@H]3CCCN3C)nc3c2cc(C)n3-c2cccc3ccccc23)C[C@@H]1CC#N ; C=C(F)C(=O)N1CCN(c2nc(OC[C@@H]3CCCN3C)nc3c2CN(c2cccc4ccccc24)C3)C[C@@H]1CC#N ; C=C(F)C(=O)N1CCN(c2nc(OC[C@@H]3CCCN3C)nc3c2CN(c2cccc4ccccc24)S3(=O)=O)C[C@@H]1CC#N ; C=C(F)C(=O)N1CCN(c2nc(OC[C@@H]3CCCN3C)nc3c2S(=O)(=O)N(c2cccc4ccccc24)C3)C[C@@H]1CC#N]

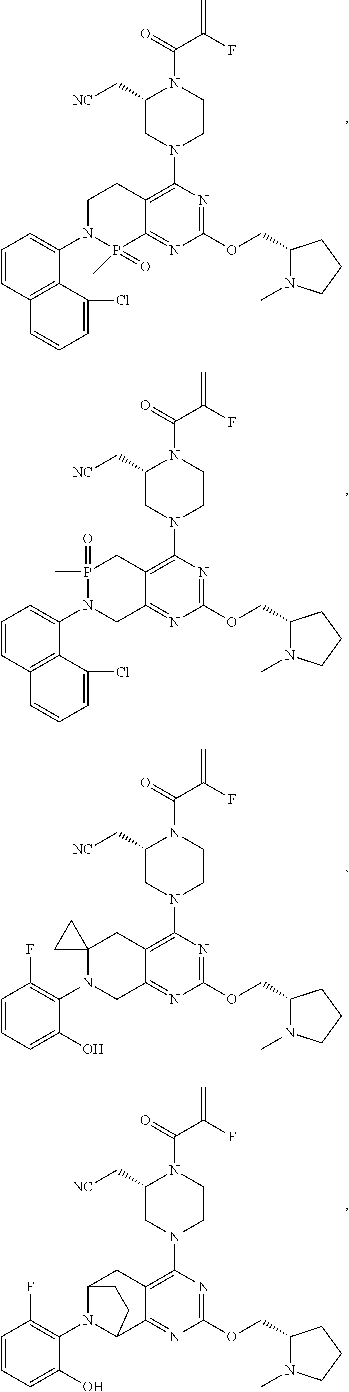 [SMILES: C=C(F)C(=O)N1CCN(c2nc(OC[C@@H]3CCCN3C)nc3c2CCN(c2cccc4cccc(Cl)c24)P3(C)=O)C[C@@H]1CC#N ; C=C(F)C(=O)N1CCN(c2nc(OC[C@@H]3CCCN3C)nc3c2CP(C)(=O)N(c2cccc4cccc(Cl)c24)C3)C[C@@H]1CC#N ; C=C(F)C(=O)N1CCN(c2nc(OC[C@@H]3CCCN3C)nc3c2CC2(CC2)N(c2c(O)cccc2F)C3)C[C@@H]1CC#N ; C=C(F)C(=O)N1CCN(c2nc(OC[C@@H]3CCCN3C)nc3c2CC2CCC3N2c2c(O)cccc2F)C[C@@H]1CC#N]

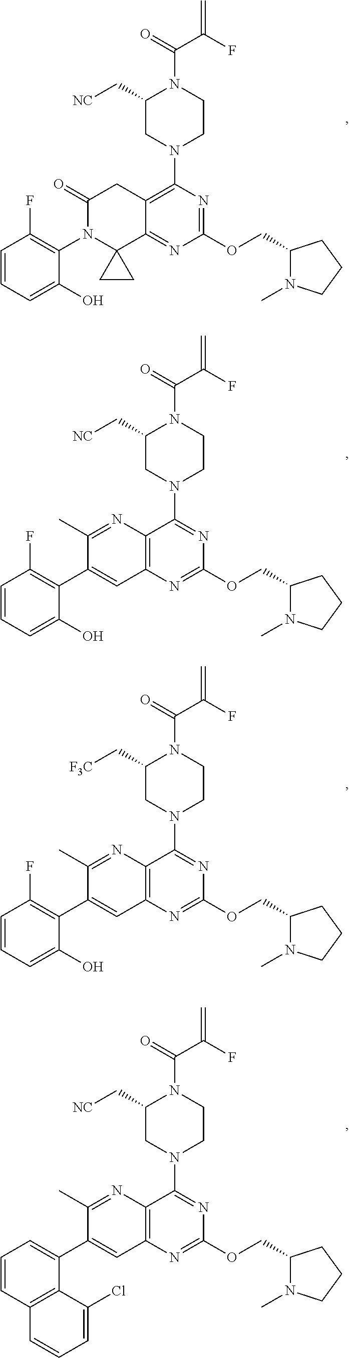 [SMILES: C=C(F)C(=O)N1CCN(c2nc(OC[C@@H]3CCCN3C)nc3c2CC(=O)N(c2c(O)cccc2F)C32CC2)C[C@@H]1CC#N ; C=C(F)C(=O)N1CCN(c2nc(OC[C@@H]3CCCN3C)nc3cc(-c4c(O)cccc4F)c(C)nc23)C[C@@H]1CC#N ; C=C(F)C(=O)N1CCN(c2nc(OC[C@@H]3CCCN3C)nc3cc(-c4c(O)cccc4F)c(C)nc23)C[C@@H]1CC(F)(F)F ; C=C(F)C(=O)N1CCN(c2nc(OC[C@@H]3CCCN3C)nc3cc(-c4cccc5cccc(Cl)c45)c(C)nc23)C[C@@H]1CC#N]

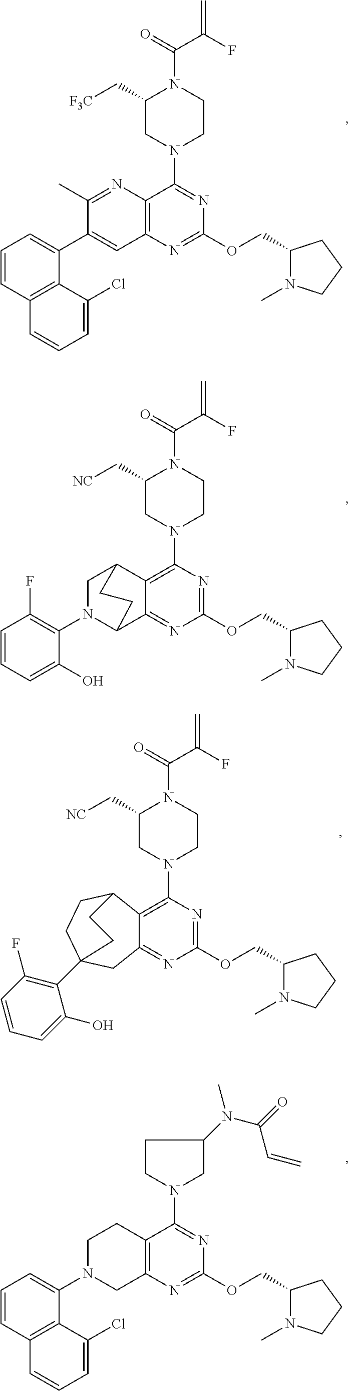 [SMILES: C=C(F)C(=O)N1CCN(c2nc(OC[C@@H]3CCCN3C)nc3cc(-c4cccc5cccc(Cl)c45)c(C)nc23)C[C@@H]1CC(F)(F)F ; C=C(F)C(=O)N1CCN(c2nc(OC[C@@H]3CCCN3C)nc3c2C2CCC3N(c3c(O)cccc3F)C2)C[C@@H]1CC#N ; C=C(F)C(=O)N1CCN(c2nc(OC[C@@H]3CCCN3C)nc3c2C2CCC(c4c(O)cccc4F)(CC2)C3)C[C@@H]1CC#N ; C=CC(=O)N(C)C1CCN(c2nc(OC[C@@H]3CCCN3C)nc3c2CCN(c2cccc4cccc(Cl)c24)C3)C1]

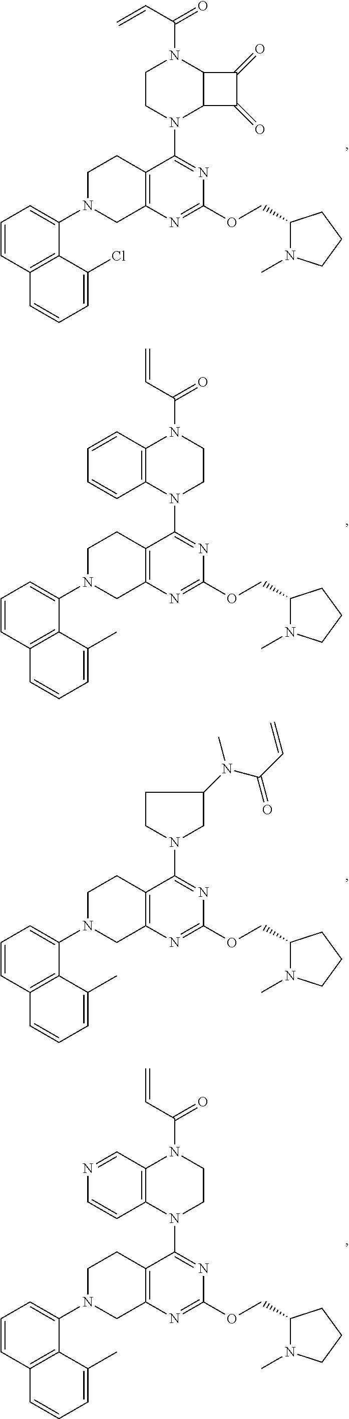 [SMILES: C=CC(=O)N1CCN(c2nc(OC[C@@H]3CCCN3C)nc3c2CCN(c2cccc4cccc(Cl)c24)C3)C2C(=O)C(=O)C21 ; C=CC(=O)N1CCN(c2nc(OC[C@@H]3CCCN3C)nc3c2CCN(c2cccc4cccc(C)c24)C3)c2ccccc21 ; C=CC(=O)N(C)C1CCN(c2nc(OC[C@@H]3CCCN3C)nc3c2CCN(c2cccc4cccc(C)c24)C3)C1 ; C=CC(=O)N1CCN(c2nc(OC[C@@H]3CCCN3C)nc3c2CCN(c2cccc4cccc(C)c24)C3)c2ccncc21]

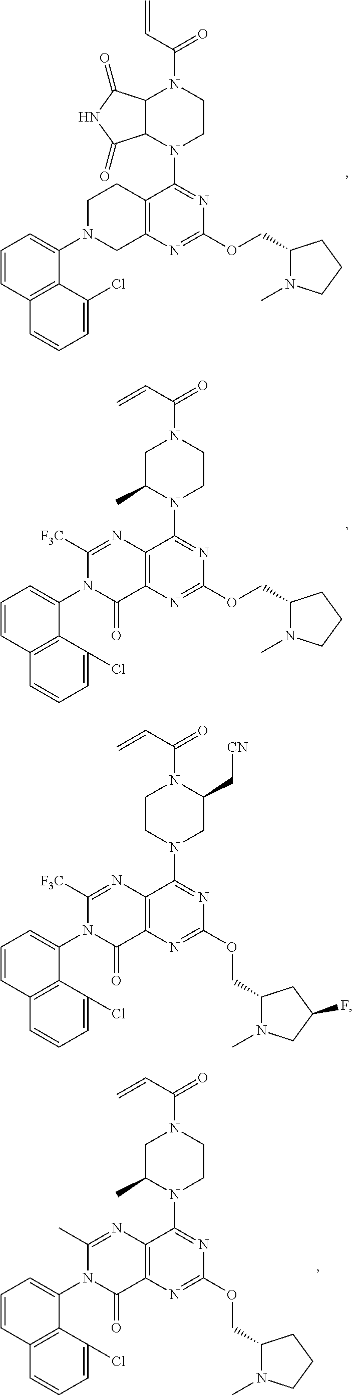

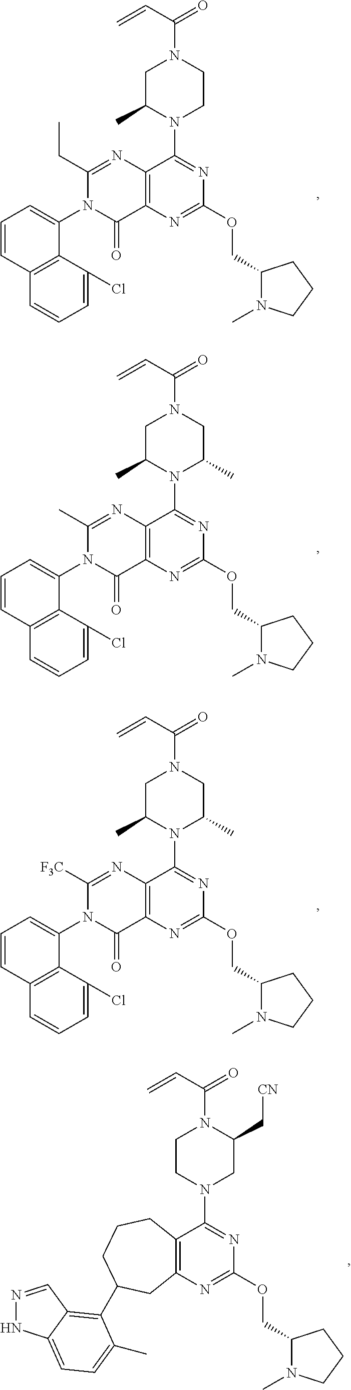

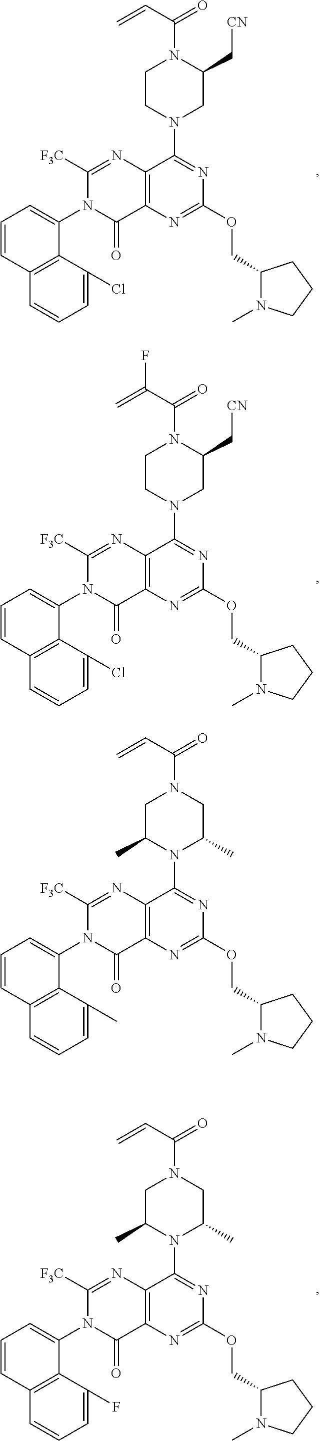

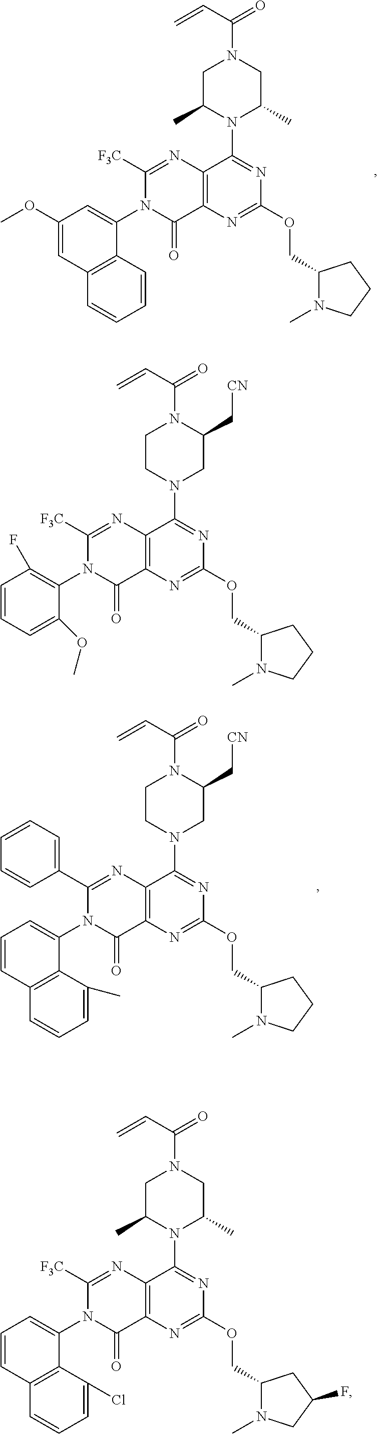

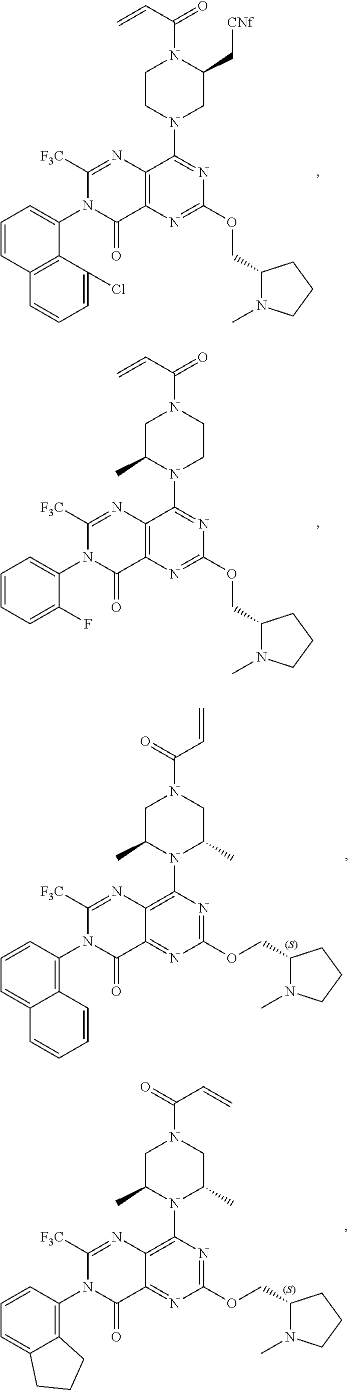

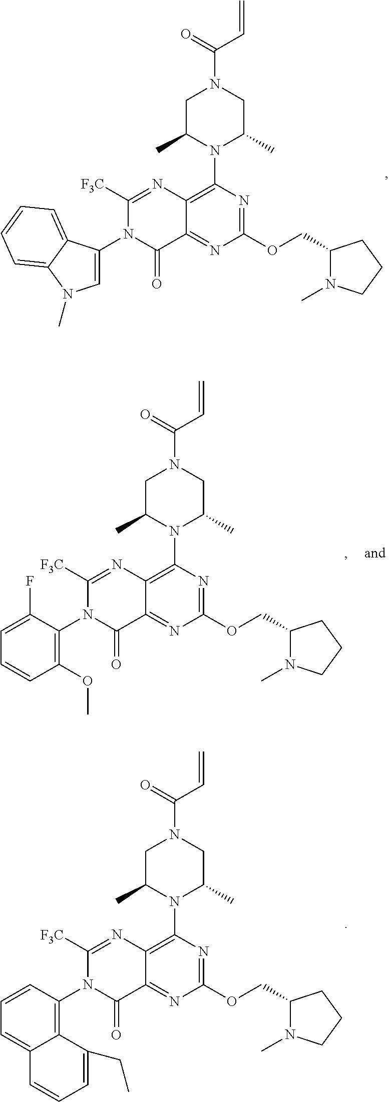 [SMILES: C=CC(=O)N1C[C@H](C)N(c2nc(OC[C@@H]3CCCN3C)nc3c(=O)n(-c4cn(C)c5ccccc45)c(C(F)(F)F)nc23)[C@@H](C)C1 ; C=CC(=O)N1C[C@H](C)N(c2nc(OC[C@@H]3CCCN3C)nc3c(=O)n(-c4c(F)cccc4OC)c(C(F)(F)F)nc23)[C@@H](C)C1 ; C=CC(=O)N1C[C@H](C)N(c2nc(OC[C@@H]3CCCN3C)nc3c(=O)n(-c4cccc5cccc(CC)c45)c(C(F)(F)F)nc23)[C@@H](C)C1]

61. A pharmaceutical composition comprising the compound of any one of claims 1-60 or a pharmaceutically acceptable salt thereof, and a pharmaceutically acceptable carrier.

62. The pharmaceutical composition of claim 61, wherein the pharmaceutical composition is formulated for oral administration.

63. The pharmaceutical composition of claim 61, wherein the pharmaceutical composition is formulated for injection.

64. A method for treating cancer, comprising administering an effective amount of a compound of any one of claims 1-60 or a pharmaceutically acceptable salt thereof or the pharmaceutical composition of any one of claims 61-63 to a subject in need thereof.

65. The method of claim 64, wherein the cancer is lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, hematological cancer, colorectal cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, MYH associated polyposis, or pituitary adenoma.

66. The method of claim 64, wherein the cancer is associated with KRas G12C mutation.

67. The method of claim 66, wherein the cancer is a hematological cancer, pancreatic cancer, MYH associated polyposis, colorectal cancer, or lung cancer.

68. A method for treating cancer in a subject in need thereof, the method comprising:
(a) determining that the cancer is associated with KRas G12C mutation; and
(b) administering to the subject an effective amount of a compound of any one of claims 1-60 or a pharmaceutically acceptable salt thereof or the pharmaceutical composition of any one of claims 61-63.

69. A method for inhibiting tumor metastasis, comprising administering an effective amount of a compound of any one of claims 1-60 or a pharmaceutically acceptable salt thereof or the pharmaceutical composition of any one of claims 61-63 to a subject in need thereof.

70. A method for regulating activity of a KRas G12C mutant protein, comprising reacting the KRas G12C mutant protein with a compound of any one of claims 1-60 or a pharmaceutically acceptable salt thereof or the pharmaceutical composition of any one of claims 61-63.

71. A method for preparing a labeled KRas G12C mutant protein, comprising reacting the KRas G12C mutant protein with a compound of any one of claims 1-60 or a pharmaceutically acceptable salt thereof, to result in the labeled KRas G12C mutant protein.

72. Use of a compound of any one of claims 1-60 or a pharmaceutically acceptable salt thereof or the pharmaceutical composition of any one of claims 61-63, in the manufacture of a medicament for treating cancer.

73. Use of a compound of any one of claims 1-60 or a pharmaceutically acceptable salt thereof or the pharmaceutical composition of any one of claims 61-63, in the manufacture of a medicament for inhibiting tumor metastasis.

74. A compound of any one of claims 1-60 or a pharmaceutically acceptable salt thereof or the pharmaceutical composition of any one of claims 61-63, for treating cancer.

75. A compound of any one of claims 1-60 or a pharmaceutically acceptable salt thereof or the pharmaceutical composition of any one of claims 61-63, for inhibiting tumor metastasis.
